(19) United States
(12) Patent Application Publication
(10) Pub. No.: US 2009/0186868 A1
(43) Pub. Date: Jul. 23, 2009

(54) Taxane Compound Having Azetidine Ring Structure

(75) Inventors: Kouichi Uoto, Tokyo (JP); Yasuyuki Takeda, Chiba (JP); Atsunobu Sakamoto, Tokyo (JP); Yoshihiro Takayanagi, Kanagawa (JP)

Correspondence Address: GREENBLUM & BERNSTEIN, P.L.C., 1950 ROLAND CLARKE PLACE, RESTON, VA, 20191, US

(73) Assignee: Daiichi Sankyo Company Limited, Tokyo (JP)

(21) Appl. No.: 12/084,065

(22) PCT Filed: Oct. 24, 2006
(86) PCT No.: PCT/JP2006/321094

§ 371 Date: Jun. 23, 2008

(30) Foreign Application Priority Data
Oct. 25, 2005 (JP) 2005-309474

(51) Int. Cl. A61K 31/397; C07D 493/04; A61K 31/443; A61K 31/427; A61K 31/422; A61K 31/506; A61P 35/00

(52) U.S. Cl. 514/210.19; 548/952; 546/283.7; 548/204; 548/236; 544/335

(57) ABSTRACT

A compound represented by the general formula (1) [X¹ and X² represent hydrogen atom, a halogen atom, hydroxyl group and the like, R¹ represents a phenyl group, R² represents an alkyl group, an alkenyl group, or an alkoxy group, R³ represents hydrogen atom, a halogen atom, hydroxyl group, or an alkoxy group, R⁴ represents hydrogen atom, or an alkyl group, Z¹ and Z² represent hydrogen atom, a halogen atom, hydroxyl group and the like, Z³ represents cyano group, an alkyl group, an alkenyl group and the like, Z⁴ represents an alkyl group, an alkenyl group, an alkynyl group and the like, and [symbol] represents a single bond or a double bond], which shows high antitumor effect against cancer cells including drug resistant cells.

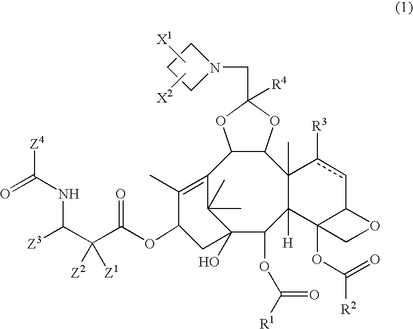

### FIELD OF THE INVENTION

The present invention relates to a taxane compound having antitumor activity. Specifically, the present invention relates to a taxane compound having an azetidine ring structure.

### BACKGROUND ART

As compounds having antitumor activity, taxane compounds are available. A typical example of the taxane compounds includes paclitaxel represented by the following general formula (1) which is a naturally occurring substance.

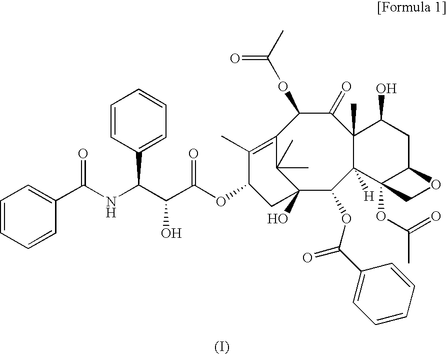

Paclitaxel is obtainable from the trunk of Taxus baccata and the like in a small amount. The mechanism of action of paclitaxel is considered to be based on inhibitory action on depolymerization of microtubules in cell division.

Several taxane compounds having an antitumor activity superior to that of paclitaxel have been reported in recent years (see, Patent documents 1 to 3).

[Patent document 1] Japanese Patent Unexamined Publication (KOKAI) No. 9-12578
[Patent document 2] International Patent Publication WOO 1/027115
[Patent document 3] Japanese Patent Unexamined Publication No. 2002-332287

### DISCLOSURE OF THE INVENTION

### Object to be Achieved by the Invention

Since anticancer agents generally have an optimum dose very close to a maximum tolerance dose (MTD), problems of side effects such as bone marrow depression and digestive organ diseases arise in many clinical cases. Patients may sometimes die from these side effects when they are serious. Therefore, development of anticancer agents has been desired which have a wide effective dose range (therapeutic range) and an optimum dose wide apart from MTD.

The present invention provides a novel taxane anticancer agent having high antitumor activity and a wider effective dose range.

### Means for Achieving the Object

The inventor of the present invention conducted various researches, and as a result, they found that the compounds represented by the following general formula (1) had high antitumor effect against cancer cells including drug resistant cells, and a wide effective dose range to accomplish the present invention.

The present invention thus provides the followings:

1. A compound represented by the general formula (1) or a salt thereof, or a solvate thereof:

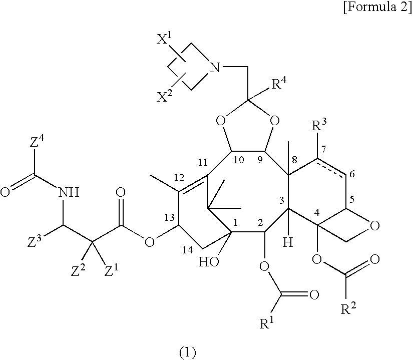

[wherein X¹ and X² independently represent hydrogen atom, a halogen atom, hydroxyl group, cyano group, carboxy group, an alkoxy group which may be substituted, an alkoxycarbonyl group, an alkyl group which may be substituted, an alkenyl group which may be substituted, an alkynyl group which may be substituted, a phenyl group which may be substituted, an alkanoyl group which may be substituted, a carbamoyl group which may be substituted, a carbamoyloxy group which may be substituted, an alkylsulfonyl group which may be substituted, an aminosulfonyl group which may be substituted, an amino group which may be substituted, or a 4- to 6-membered saturated heterocyclic group, or

X¹ and X² may together form oxo group or a ═N—OY group (Y represents hydrogen atom, or an alkyl group which may be substituted), or X¹ and X² may form a 4- to 6-membered saturated hydrocarbon ring binding as a spiro ring system or a condensed ring system, or a 5- or 6-membered saturated heterocyclic ring binding as a spiro ring system or a condensed ring system together with the carbon atom or atoms to which X¹ and X² bind,

R¹ represents a phenyl group which may be substituted,

R² represents an alkyl group which may be substituted, an alkenyl group which may be substituted, or an alkoxy group which may be substituted,

R³ represents hydrogen atom, a halogen atom, hydroxyl group, or an alkoxy group which may be substituted,

R⁴ represents hydrogen atom, or an alkyl group which may be substituted,

Z¹ and Z² independently represent hydrogen atom, a halogen atom, hydroxyl group, an alkoxy group which may be substituted, or an alkyl group which may be substituted,

Z³ represents cyano group, an alkyl group which may be substituted, an alkenyl group which may be substituted, an alkynyl group which may be substituted, a phenyl group which may be substituted, or a 4- to 6-membered saturated or unsaturated heterocyclic group which may be substituted,

Z⁴ represents an alkyl group which may be substituted, an alkenyl group which may be substituted, an alkynyl group which may be substituted, a phenyl group which may be substituted, an aralkyl group which may be substituted, a 5- or 6-membered aromatic heterocyclic group which may be substituted, or an alkoxy group which may be substituted, and

the 6- and 7-positions of the partial structure represented by the following general formula (2)

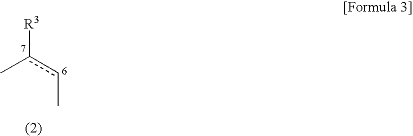

may be bonded with a double bond (when the bond is a double bond, R³ is hydrogen atom);

2. A compound represented by the general formula (1) having the absolute configuration represented by the general formula (3) or a salt thereof, or a solvate thereof:

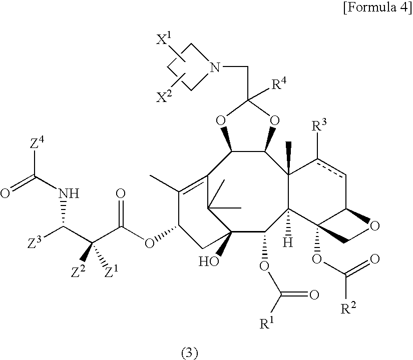

(wherein X¹, X², R¹ to R⁴, and Z¹ to Z⁴ have the same meanings as those defined above);

3. A medicament comprising a compound represented by the aforementioned general formula (1) or (3) or a salt thereof, or a solvate thereof as an active ingredient;

4. An anticancer agent comprising a compound represented by the aforementioned general formula (1) or (3) or a salt thereof, or a solvate thereof as an active ingredient;

5. A pharmaceutical composition comprising a compound represented by the aforementioned general formula (1) or (3) or a salt thereof, or a solvate thereof, and a pharmaceutically acceptable carrier;

6. Use of a compound represented by the aforementioned general formula (1) or (3) or a salt thereof, or a solvate thereof for the manufacture of the medicament;

7. Use of a compound represented by the aforementioned general formula (1) or (3) or a salt thereof, or a solvate thereof for the manufacture of the anticancer agent;

8. A method for therapeutic treatment of a cancer, which comprises administering a compound represented by the aforementioned general formula (1) or (3) or a salt thereof, or a solvate thereof.

### EFFECT OF THE INVENTION

The compounds of the present invention have high antitumor effect against cancer cells including drug resistant cells. Accordingly, they can be used as potent anticancer agents. Furthermore, the compounds of the present invention have a wide range of effective dose, thereby an optimum dose widely apart from MTD is expected, and therefore they can be used as safe anticancer agents hardly causing side effects.

### BEST MODE FOR CARRYING OUT THE INVENTION

The substituents in the general formula (1) are explained below.

Examples of the “halogen atom” include fluorine atom, chlorine atom, and bromine atom.

The “alkoxy group which may be substituted” means an alkoxy group having a linear, branched or cyclic alkyl group having 1 to 6 carbon atoms, which may have one or more substituents. Examples of the alkoxy group include, for example, methoxy group, ethoxy group, propoxy group, isopropoxy group, butoxy group, and cyclopentyloxy group. Examples of the substituent which can substitute on these alkoxy groups include, for example, a halogen atom.

The “alkoxycarbonyl group” means a group formed by carbonyl group (—CO—) bound with an alkoxy group having a linear, branched or cyclic alkyl group having 1 to 6 carbon atoms, and examples include, for example, methoxycarbonyl group, ethoxycarbonyl group, and propylcarbonyl group.

The “alkyl group which may be substituted” means a linear, branched or cyclic alkyl group which may have one or more substituents, and examples include, for example, an alkyl group having 1 to 6 carbon atoms, which may have one or more substituents, and a cycloalkyl group having 3 to 8 carbon atoms, which may have one or more substituents. Examples of the alkyl group include, for example, methyl group, ethyl group, propyl group, isopropyl group, cyclopropyl group, cyclobutyl group, cyclopentyl group, and cyclohexyl group. Examples of the substituent which can substitute on this alkyl group include, for example, a halogen atom, hydroxyl group, an alkoxy group, carbamoyl group, amino group, an alkanoyl group, and cyano group, and the number of the substituent which can substitute on the alkyl group is preferably 1 to 3.

The “alkenyl group which may be substituted” means a linear or branched alkenyl group having 2 to 6 carbon atoms, which may have one or more substituents. Examples of the alkenyl group include, for example, vinyl group, allyl group, and butenyl group. Examples of the substituent which can substitute on the alkenyl group include, for example, a halogen atom, hydroxyl group, an alkoxy group, carbamoyl group, amino group, an alkanoyl group, and cyano group, and the number of the substituent which can substitute on the alkenyl group is preferably 1 to 3.

The “alkynyl group which may be substituted” means a linear or branched alkynyl group having 2 to 6 carbon atoms, which may have one or more substituents. Examples of the alkynyl group include, for example, ethynyl group, propynyl group, butynyl group, pentynyl group, and hexynyl group. Examples of the substituent which can substitute on this alkynyl group include, for example, a halogen atom, hydroxyl group, an alkoxy group, carbamoyl group, amino group, an alkanoyl group, and cyano group, and the number of the substituent which can substitute on this alkynyl group is preferably 1 to 3.

The “phenyl group which may be substituted” means unsubstituted phenyl group or a substituted phenyl group. In the case of the substituted phenyl group, examples of the substituent include, for example, a halogen atom, a hydroxyl group, an alkoxy group, an alkyl group, carbamoyl group, amino group, an alkanoyl group, and cyano group, and the number of the substituent which can substitute on the phenyl group is preferably 1 to 3, more preferably 1 or 2.

The “alkanoyl group which may be substituted” means an alkanoyl group which may have one or more substituents. Examples of the alkanoyl group include, for example, formyl group, acetyl group, methylpropionyl group, and cyclopentanecarbonyl group. Examples of the substituent which can substitute on the alkanoyl group include, for example, a halogen atom and amino group.

The “carbamoyl group which may be substituted” means a carbamoyl group which may have one or two substituents. Examples of the substituent which can substitute on this carbamoyl group include, for example, an alkyl group having 1 to 6 carbon atoms.

The “carbamoyloxy group which may be substituted” means a carbamoyloxy group which may have one or two substituents. Examples of the substituent which can substitute on this carbamoyloxy group include, for example, an alkyl group having 1 to 6 carbon atoms.

The “alkylsulfonyl group which may be substituted” means a group formed by sulfonyl group (—SO₂—) bound with an alkyl group having 1 to 6 carbon atoms, which may have one or more substituents. Examples of the alkylsulfonyl group include, for example, methanesulfonyl group and ethanesulfonyl group. Examples of the substituent which can substitute on the alkylsulfonyl group include, for example, carboxy group and an alkoxycarbonyl group.

The “aminosulfonyl group which may be substituted” means a group formed by sulfonyl group (—SO₂—) bound with an amino group which may have one or more substituents. Examples of the aminosulfonyl group include, for example, methylaminosulfonyl group, ethylaminosulfonyl group, dimethylaminosulfonyl group, and diethylaminosulfonyl group.

The “amino group which may be substituted” means amino group or an amino group substituted with one or two substituents. Examples of the substituent which can substitute on the amino group include, for example, an alkyl group, an alkanoyl group, an alkylsulfonyl group, an alkoxycarbonyl group, carbamoyl group, an alkylcarbamoyl group, and a dialkylcarbamoyl group, more specifically, methyl group, ethyl group, acetyl group, methanesulfonyl group, ethanesulfonyl group, methoxycarbonyl group, ethoxycarbonyl group, carbamoyl group, methylcarbamoyl group, ethylcarbamoyl group, dimethylcarbamoyl group, and diethylcarbamoyl group.

The “4- to 6-membered saturated heterocyclic group” means a substituent derived from a 4- to 6-membered saturated heterocyclic compound containing one or X² bind, means a ring formed by an alkyleneoxy group or alkyleneoxyalkylene group having 3 or 4 carbon atoms binding to the carbon atom of the azetidine ring in the general formula (1) as a spiro ring system. Specifically, the azetidine ring in the general formula (1) and the saturated heterocyclic ring form a substituent derived from, for example, 5-oxa-2-aza-spiro[3.4]octane, 6-oxa-2-aza-spiro[3.4]octane, 5-oxa-1-aza-spiro[3.4]octane, 6-oxa-1-aza-spiro[3.4]octane, 5-oxa-2-aza-spiro[3.5]nonane, 6-oxa-2-aza-spiro[3.5]nonane, 7-oxa-2-aza-spiro[3.5]nonane, 5-oxa-1-aza-spiro[3.5]nonane, 6-oxa-1-aza-spiro[3.5]nonane, or 7-oxa-1-aza-spiro[3.5]nonane.

The “5- or 6-membered saturated heterocyclic ring binding as a condensed ring system” formed by X¹ and X², together with the carbon atoms of the ring to which X¹ and X² bind, means a ring formed by an alkyleneoxy group or alkyleneoxyalkylene group having 2 or 3 carbon atoms binding to the carbon atoms of the azetidine ring in the general formula (1) as a condensed ring system. Specifically, the azetidine ring in the general formula (1) and the saturated heterocyclic ring form a substituent derived from, for example, 2-oxa-7-aza-bicyclo[3.2.0]heptane, 3-oxa-6-aza-bicyclo[3.2.0]heptane, 2-oxa-6-aza-bicyclo[3.2.0]heptane, 2-oxa-8-aza-bicyclo[4.2.0]octane, 3-oxa-8-aza-bicyclo[4.2.0]octane, 3-oxa-7-aza-bicyclo[4.2.0]octane, or 2-oxa-7-aza-bicyclo[4.2.0]octane.

The “4- to 6-membered saturated or unsaturated heterocyclic group which may be substituted” means a substituent derived from a 4- to 6-membered saturated or unsaturated heterocyclic compound containing one or more oxygen atoms, nitrogen atoms, or sulfur atoms as constituent atoms of the ring structure, which may have one or more substituents. This heterocyclic group may bind at any position. Examples of the 4- to 6-membered saturated or unsaturated heterocyclic group include, for example, a group derived from pyridine, pyrimidine, pyrrole, furan, thiophene, imidazole, pyrazole, oxazole, thiazole, isothiazole, oxadiazole, triazole, oxetane, tetrahydrofuran, or the like. When this heterocyclic group has a substituent, examples of the substituent include, for example, one or more halogen atoms, alkyl groups, amino groups, and alkoxy groups.

The “aralkyl group which may be substituted” means an aralkyl group which more oxygen atoms, nitrogen atoms or sulfur atoms as constituent atoms of a ring structure, and this heterocyclic group may bind at any position. Examples of the 4- to 6-membered saturated heterocyclic group include, for example, a substituent derived from a saturated heterocyclic compound such as pyrrolidine, tetrahydrofuran, tetrahydrothiophene, imidazolidine, tetrahydropyran, piperidine, piperazine, dioxane, pyrazolidine, morpholine, azetidine, and oxetane. When this saturated heterocyclic group has a substituent, examples of the substituent include, for example, one or more halogen atoms, alkyl groups, amino groups, and alkoxy groups.

“Y” in the “═N—OY group (Y represents hydrogen atom or an alkyl group which may be substituted)” is hydrogen atom, or a linear, branched, or cyclic alkyl group having 1 to 6 carbon atoms, which may have one or more substituents. Typical examples of the “═N—OY group” include, for example, ═N—O—H group, ═N—O-methyl group, ═N—O-ethyl group, ═N—O-propyl group, and ═N—O-cyclopropyl group.

The “4- to 6-membered saturated hydrocarbon ring binding as a spiro ring system” formed by X¹ and X², together with the carbon atom of the ring to which X¹ and X² bind, means a ring formed by an alkylene group having 3 to 5 carbon atoms binding to the carbon atom of the azetidine ring in the general formula (1) as a spiro ring system. Specifically, the azetidine ring in the general formula (1) and the saturated hydrocarbon ring form a substituent derived from, for example, 2-aza-spiro[3.3]heptane, 1-aza-spiro[3.3]heptane, 2-aza-spiro[3.4]octane, 1-aza-spiro[3.4]octane, 2-aza-spiro[3.5]nonane, or 1-aza-spiro[3.5]nonane.

The “4- to 6-membered saturated hydrocarbon ring binding as a condensed ring system” formed by X¹ and X², together with the carbon atoms of the ring to which X¹ and X² bind, means a ring formed by an alkylene group having 2 to 4 carbon atoms binding to the carbon atoms of the azetidine ring in the general formula (1) as a condensed ring system. Specifically, the azetidine ring in the general formula (1) and the saturated hydrocarbon ring form a substituent derived from, for example, 2-aza-bicyclo[2.2.0]hexane, 6-aza-bicyclo[3.2.0]heptane, or 7-aza-bicyclo[4.2.0]octane.

The “5- or 6-membered saturated heterocyclic ring binding as a spiro ring system” formed by X¹ and X², together with the carbon atom of the ring to which X¹ and may have one or more substituents. Examples of the aralkyl group include, for example, benzyl group and phenethyl group. When the aralkyl group has a substituent, examples of the substituent include, for example, a halogen atom, hydroxyl group, an alkoxy group, amino group, and an alkyl group.

In an embodiment of the present invention, X¹ and X² preferably independently represent hydrogen atom, a halogen atom, hydroxyl group, cyano group, an alkoxy group which may be substituted, an alkyl group which may be substituted, a carbamoyl group which may be substituted, a carbamoyloxy group which may be substituted, an alkylsulfonyl group which may be substituted, or an amino group which may be substituted, or X¹ and X² together represent a ═N—OY group (Y represents hydrogen atom or an alkyl group which may be substituted).

In another embodiment of the present invention, X¹ and X² more preferably independently represent hydrogen atom, a halogen atom, hydroxyl group, cyano group, a lower alkoxy group, a lower alkyl group, a hydroxy(lower alkyl) group, a (lower alkoxy)(lower alkyl) group, a halogenated lower alkyl group, a cycloalkyl group having 3 to 8 carbon atoms, a di(lower alkyl)carbamoyl group, carbamoyloxy group, a di(lower alkyl)carbamoyloxy group, a (lower alkyl)sulfonyl group, a N-(lower acyl)-N-(lower alkyl)amino group, a N-(lower alkyl)sulfonyl-N-(lower alkyl)amino group, a N-(lower alkoxy)carbonyl-N-(lower alkyl)amino group, or a N,N-di(lower alkyl)amino group, or X¹ and X² together represent a ═N—O-(lower alkyl) group. The term “lower” used for the “lower alkoxy group”, “lower alkyl group”, “hydroxy(lower alkyl) group”, and the like means that the number of carbon atoms of these groups is 1 to 6.

In another embodiment of the present invention, X¹ and X² still more preferably independently represent hydrogen atom, fluorine atom, hydroxyl group, cyano group, methoxy group, methyl group, ethyl group, hydroxymethyl group, methoxymethyl group, trifluoromethyl group, cyclopropyl group, dimethylcarbamoyl group, carbamoyloxy group, dimethylcarbamoyloxy group, methanesulfonyl group, N-acetyl-N-methylamino group, N-methanesulfonyl-N-methylamino group, N-methoxycarbonyl-N-methylamino group, or N,N-dimethylamino group, or X¹ and X² together represent ═N—O-methyl group.

In an embodiment of the present invention, R¹ is preferably unsubstituted phenyl group. A phenyl group substituted with one or two fluorine atoms, chlorine atoms, methyl groups, or methoxy groups at the meta-position or meta-positions is also preferred.

In an embodiment of the present invention, R² is preferably an alkyl group which may be substituted, more preferably methyl group.

In an embodiment of the present invention, R³ is preferably hydrogen atom, fluorine atom, or hydroxyl group, more preferably hydrogen atom.

In an embodiment of the present invention, R⁴ is preferably hydrogen atom.

In an embodiment of the present invention, Z¹ is preferably a halogen atom or hydroxyl group, more preferably hydroxyl group.

In an embodiment of the present invention, Z² is preferably hydrogen atom, a halogen atom, or an alkyl group which may be substituted, more preferably hydrogen atom or methyl group.

In an embodiment of the present invention, examples of preferred combinations of Z¹ and Z² include, combination of hydroxyl group as Z¹ and hydrogen atom as Z², combination of hydroxyl group as Z¹ and methyl group as Z², and combination of fluorine atoms as Z¹ and Z².

In an embodiment of the present invention, Z³ is preferably an alkyl group which may be substituted, or a 4- to 6-membered saturated or unsaturated heterocyclic group which may be substituted. Z³ is more preferably an unsubstituted alkyl group, an alkyl group substituted with a halogen atom, unsubstituted pyrimidinyl group, unsubstituted pyridyl group, or a pyridyl group substituted with a halogen atom. Z³ is still more preferably an alkyl group substituted with fluorine atom, unsubstituted pyrimidinyl group, or a pyridyl group substituted with fluorine atom.

In an embodiment of the present invention, Z⁴ is preferably a phenyl group which may be substituted, or an alkoxy group which may be substituted.

The 6- and 7-positions of the partial structure represented by the following general formula (2)

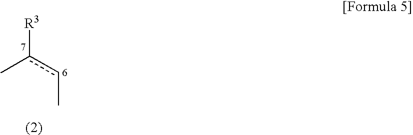

are meant to be optionally bound with a double bond (when the bond is a double bond, R³ is hydrogen atom)

In an embodiment of the present invention, the dotted line moiety between the 6- and 7-positions is preferably a single bond.

In the present invention, the compound is preferably in the absolute configuration represented by the general formula (3).

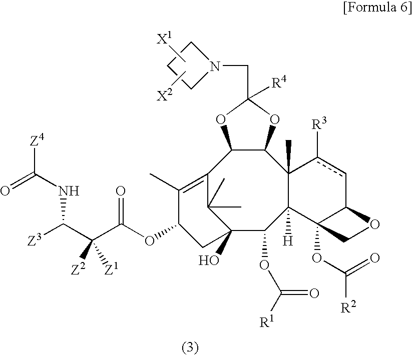

The configuration of the 3′-position to which the substituent Z³ binds is more preferably the same configuration as that of natural paclitaxel. When the bond between the 6- and 7-positions is a single bond, the configuration of R³ at the 7-position may be either of α- or β-configuration.

Stereoisomers or optical isomers based on asymmetric carbon atoms of the compounds of the present invention represented by the general formula (1) may exist, and any of these stereoisomers and optical isomers, and mixtures thereof fall within the scope of the present invention. Preferred examples of the compounds of the present invention represented by the general formula (1) include those in the absolute configuration represented by the general formula (3).

Salts of the compounds of the present invention represented by the general formula (1) are not particularly limited so long as they are medically acceptable salts, including acid addition salts or salts of carboxy group, for example, although the taxane derivatives of the present invention may be used in a free form.

Examples of the acid addition salts include inorganic acid salts such as hydrochlorides, sulfates, nitrates, hydrobromides, hydroiodides and phosphates, and organic acid salts such as acetates, methanesulfonates, benzenesulfonates, toluenesulfonates, citrates, maleates, fumarates, and lactates.

Examples of the salts of carboxy group include, for example, alkali metal salts such as lithium salts, sodium salts, and potassium salts, alkaline earth metal salts such as magnesium salts and calcium salts, ammonium salts, triethylamine salts, N-methylglucamine salts, tris(hydroxymethyl)aminomethane salts, and the like, and they may be inorganic salts or organic salts.

Methods for preparing the compounds represented by the general formula (3) will be explained below as typical examples of the compounds represented by the general formula (1). The compounds of the present invention can be prepared by various methods, and the preparation methods shown below are mere examples. Therefore, the present invention should not be construed to be limited to these examples.

When the reactions are performed, the reactions can be carried out with protecting substituents with appropriate protective groups as required, and types of the protective groups and the order of conversions of the substituents are not particularly limited.

### [Preparation Method 1]

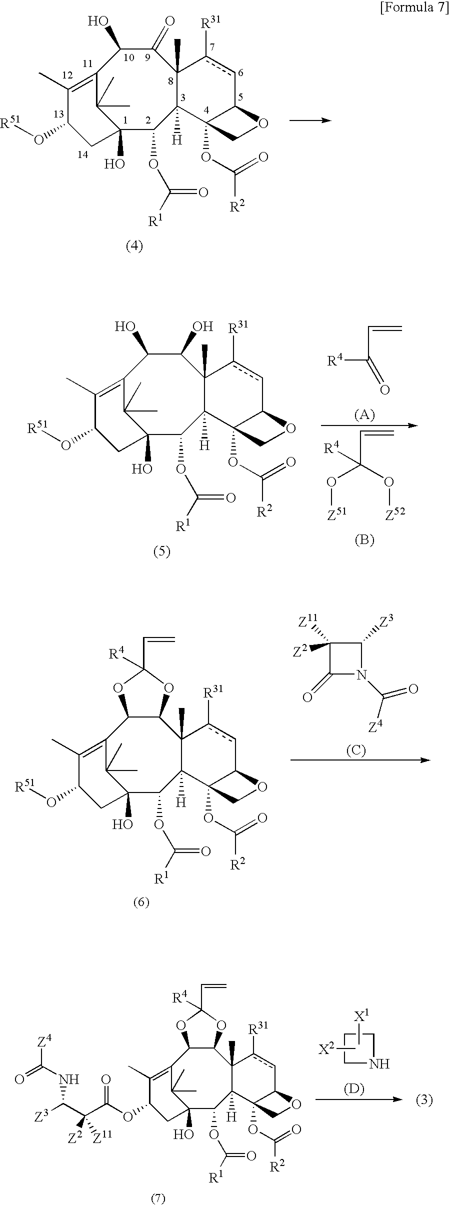

[In the formulas, R³¹ means R³ or R³ protected with a protective group (when R³ is hydroxyl group). R⁵¹ means hydrogen atom or a protective group of hydroxyl group. Z⁵¹ and Z⁵² mean an alkyl group, respectively. Z¹¹ means Z¹ or Z¹ protected with a protective group (when Z¹ is hydroxyl group). X¹, X², R¹ to R⁴, and Z¹ to Z⁴ have the same meanings as those defined above.]

Examples of the protective group of hydroxyl group include a silyl type protective group such as trimethylsilyl group, triethylsilyl group, triisopropylsilyl group and tert-butyldimethylsilyl group, 2,2,2-trichloroethoxycarbonyl group, benzyloxycarbonyl group, benzyl group, and the like.

First, the 9-oxo group of a compound represented by the formula (4) (henceforth referred to as the compound (4), and the compounds represented by the other formulas are also referred to in a similar manner) can be treated with diborane or a metal borohydride compound such as tetra-n-butylammonium borohydride in a solvent such as methylene chloride and tetrahydrofuran according to, for example, a method described in literature (Bioorg. Med. Chem. Lett., 2002, 12, 2815, or Japanese Patent Unexamined Publication No. 2003-342269) to obtain the compound (5) having 9 β-hydroxy group.

The compound (5) and the compound (A) or compound (B) can be reacted in a solvent such as methylene chloride, tetrahydrofuran, and 1,4-dioxane in the presence of an acidic catalyst such as (±)-camphor-10-sulfonic acid, p-toluenesulfonic acid or zinc chloride according to, for example, a method described in literature (Bioorg. Med. Chem. Lett., 2003, 13,185) to obtain the compound (6).

In order to obtain the compound (7) from the compound (6), when the substituent at the 13-position, R⁵¹, is a protective group, deprotection can be first performed to obtain a compound having hydroxyl group at the 13-position, and then the compound can be condensed with the compound (C) as a racemate or optically active substance by using a base such as sodium hexamethyldisilazane, lithium hexamethyldisilazane and sodium hydride in a solvent such as ether and tetrahydrofuran according to, for example, a method described in literature (Tetrahedron, 1992, 48, 6985).

The compound (3) can be prepared from the compound (7) as follows. First, the double bond of the 9,10-propenylidenedioxy moiety of the compound (7) is treated with a catalytic amount of osmium tetroxide in a solvent (water, acetone, tetrahydrofuran, ether, pyridine, 1,4-dioxane, mixtures of these solvents, and the like) in the presence of an oxidizing agent such as 4-methylmorpholine N-oxide to obtain a diol compound, the diol compound is converted into a ketone or aldehyde by a treatment with sodium metaperiodate in a solvent (water, ether, tetrahydrofuran, methanol, ethanol, 1,4-dioxane, mixed solvent of these, and the like), and the ketone or aldehyde is treated together with an azetidine derivative (D) in a solvent (water, tetrahydrofuran, methanol, ethanol, 1,4-dioxane, methylene chloride, mixed solvent of these, and the like) in the presence of a reducing agent such as sodium borohydride, sodium cyanoborohydride, sodium triacetoxyborohydride, and lithium aluminum hydride to obtain the compound (3) of the present invention introduced with any one of various azetidino groups. By converting a substituent of the resulting compound (3) or performing deprotection for protective groups as required, another compound (3) of the present invention can be obtained.

The compounds of the present invention prepared as described above can be isolated and purified by known methods such as extraction, precipitation, fractionation chromatography, fractional crystallization, and recrystallization. Further, the compounds of the present invention can be derived into desired salts by subjecting the compounds to a usual salt formation reaction.

The compounds (3) of the present invention can also be prepared by the following method.

### [Preparation Method 2]

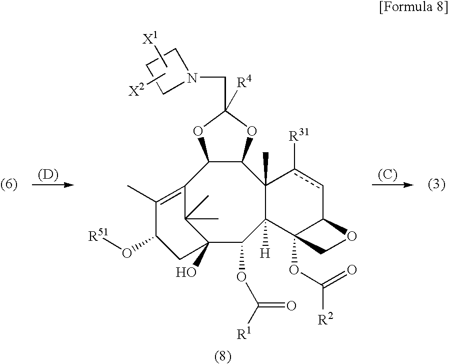

[In the formula, X¹, X², R¹, R², R³¹, R⁴, and R⁵¹ have the same meanings as those defined above.]

The compound (8) can be obtained from the compound (6) of which substituent at the 13-position, i.e., R⁵¹, is a protective group, by using reagents and conditions similar to those used for the method of converting the double bond of the 9,10-propenylidenedioxy moiety of the compound (7) into various azetidino groups of the compounds (3) described in Preparation method 1.

The compound (3) of the present invention can be obtained from the compound (8) by first performing deprotection when the substituent at the 13-position, R⁵¹, is a protective group to obtain a compound having hydroxyl group at the 13-position, then converting the 13-position according to the method for preparing the compound (7) from the compound (6) described in Preparation method 1, and performing conversion of a substituent and deprotection for a protective group as required.

Conversion of R¹, R², or R³¹ into any other substituents among those defined for the groups (for example, conversion of phenyl group into fluorophenyl group) can be performed by using a usual organic chemical method, and examples thereof are mentioned below.

The methods of converting R¹ include, for example, a method of selectively hydrolyzing the benzoyl group at the 2-position according to a method described in literature (Tetrahedron Lett., 1994, 35, 8931), and then acylating the resultant, and the compounds of which R¹ is a group other than phenyl can be obtained by this method.

The methods of converting R² include, for example, a method of selectively hydrolyzing the acetyl group at the 4-position according to a method described in literature (J. Med. Chem., 1995, 38, 2263), and then acylating the resultant, and the compounds of which R² is a group other than methyl can be obtained by this method.

The compound of which R³¹ is hydrogen atom and the bond between the 6- and 7-positions is a double bond can be obtained by, for example, reacting the compound having hydroxyl group at the 7-position and a single bond between the 6- and 7-positions with trifluoromethanesulfonic acid anhydride in a solvent such as methylene chloride in the presence of a base such as N,N-dimethylaminopyridine, and then treating the resultant with a base such as 1,8-diazabicyclo[5.4.0]-7-undecene, according to the method described in WO2001/27115.

The compound of which R³¹ is fluorine atom and the bond between the 6- and 7-positions is a single bond can be obtained by, for example, treating the compound having hydroxyl group at the 7-position and a single bond between the 6- and 7-positions with a fluorinating reagent such as (diethylamino)sulfur trifluoride in a solvent such as methylene chloride according to a method described in literature (Tetrahedron Lett., 1995, 36, 1609).

The compound of which R³¹ is methoxy group and the bond between the 6- and 7-positions is a single bond can be obtained by, for example, treating the compound having hydroxyl group at the 7-position and a single bond between the 6- and 7-positions with methyl trifluoromethanesulfonate in a basic solvent such as 2,6-di-tert-butylpyridine according to a method described in literature (Bioorg. Med. Chem. Lett., 2002, 12, 2815).

The compound of which R³¹ is hydrogen atom and the bond between the 6- and 7-positions is a single bond can be obtained by, for example, treating the compound having hydroxyl group at the 7-position and a single bond between the 6- and 7-positions with 1,1′-thiocarbonyldiimidazole in a solvent such as tetrahydrofuran in the presence of a base such as 1,8-diazabicyclo[5.4.0]-7-undecene to derive the compound into the compound of which 7-hydroxyl group is (imidazolyl)thiocarbonylated, and treating the resultant with tributyltin hydride, tris(trimethylsilyl)silane hydride, or the like in an inert solvent such as tetrahydrofuran and 1,4-dioxane in the presence of a radical initiator such as 2′,2′-azobis(isobutyronitrile) and benzoyl peroxide according to a method described in literature (Bioorg. Med. Chem. Lett., 2002, 12, 2815).

Further, the compound of which R³¹ is hydrogen atom and the bond between the 6- and 7-positions is a single bond can also be obtained by, for example, hydrogenating the compound of which R³¹ is hydrogen atom and bond between the 6- and 7-positions is a double bond by catalytic hydrogenation in a solvent such as tetrahydrofuran, 1,4-dioxane, methanol and ethanol in the presence of a catalyst such as rhodium/alumina, palladium hydroxide/carbon, palladium/carbon, and platinum oxide according to a method described in literature (Chem. Pharm. Bull., 2002, 50, 1398).

The compound (4) as the starting material for the preparation may be a marketed product, 10-deacetyl baccatin III, or can be synthesized from 10-deacetyl baccatin III. For example, the compound wherein R³¹ is fluorine atom, and R⁵¹ is triethylsilyl group, and the compound wherein R³¹ and R⁵¹ are hydrogen atoms are described in literature (Bioorg. Med. Chem. Lett., 2002, 12, 2815).

The compound (A) and the compound (B) as the starting materials for the preparation may be marketed products, or can be synthesized according to a method described in literature (J. Am. Chem. Soc., 1938, 60, 1160 or Tetrahedron Lett., 1976, 17, 2935).

As for the general synthetic method of racemic or optically active compound (C) as the starting material for the preparation, the compound can be synthesized according to a method described in literature (Tetrahedron Lett., 1993, 34, 4149).

The compound (D) as the starting material for the preparation may be a marketed product, or can be synthesized according to the method described in the reference examples.

The compounds of the present invention can be used for therapeutic treatment of various cancers such as lung cancer, gastrointestinal cancer, ovarian cancer, uterine cancer, breast cancer, hepatic cancer, head and neck cancer, blood cancer, renal cancer, and testicular tumor. In particular, the compounds of the present invention are expected to be effective against cancers of the digestive system, e.g., colon cancer, which are hardly treated therapeutically by using conventional anticancer agents.

As causes involved in acquisition of drug resistance, various causes have been elucidated. In the case of anticancer agents, an example of the causes includes expression of P-glycoprotein (P-gp) having an action of extracellularly discharging drugs. For example, it has been reported that anticancer agents such as paclitaxel and docetaxel are likely to be affected by P-gp.

Pharmaceutical compositions containing the compounds of the present invention can be administered as various injections such as intravenous injection, intramuscular injection and subcutaneous injection, or by various methods such as oral administration and dermal administration. Among these administration methods, intravenous administration of an aqueous preparation and oral administration are preferred. The aqueous preparations can be prepared by forming an acid addition product with a pharmacologically acceptable acid, or a salt with an alkali metal such as sodium. In the case of oral administration, they may be used in the form of a free compound or a salt.

As for methods for manufacturing the preparations, the preparations can be manufactured according to ordinarily applied methods for preparing various pharmaceutical preparations by choosing a suitable form depending on a method for administration. Among dosage forms of the antitumor agent of the present invention, examples of oral preparations include, for example, tablet, powder, granule, capsule, solution, syrup, elixir, oily or aqueous suspension, and the like. In the case of injections, stabilizers, preservatives, dissolving aids, and the like may be used in the preparations. A solution which optionally contains such auxiliary agents may be stored in a container and made into solid preparation by lyophilization or the like to obtain a formulation to be prepared just before use. A unit dosage for single administration may be packed in a single container or units for several administrations may be packed in a single container.

Examples of the solid preparations include, for example, tablets, capsules, granules, pills, troches, and powders. Such solid preparations may also contain pharmaceutically acceptable additives together with the compounds of the present invention. Examples of the additives include, for example, fillers, bulking agents, binders, disintegrating agents, dissolution enhancers, wetting agents, and lubricants, and these additives can be chosen and mixed as required to form the preparations.

Examples of liquid preparations include solutions, elixirs, syrups, suspensions, emulsions, and the like. Such liquid preparations may also contain pharmaceutically acceptable additives together with the compounds of the present invention. Examples of the additives include, for example, suspending agents and emulsifiers, and these additives can be chosen and mixed as required to form the preparations.

The compounds of the present invention can be used for therapeutic treatment of cancers of mammals, especially humans. Doses and administration intervals may be suitably chosen by medical practitioners depending on a site of the disease, body height and weight, sexuality, or pathological history of a patient. When the compounds are administered to humans, they are preferably administered once or 2 to 4 times a day as divided portions, and the administration is preferably repeated with appropriate intervals. Doses are preferably in a range of about 0.5 to 500 mg, preferably about 1 to 300 mg, more preferably about 1 to 100 mg, per 1 m² of body surface area for a single administration. A daily dose may exceed the aforementioned doses, if necessary, as determined by medical practitioners.

### EXAMPLES

The present invention will be specifically explained with reference to the following examples. However, the present invention is not limited to these examples, and these examples should not be construed in any limitative manner. Reagents, solvents and starting materials mentioned in the specification without particular explanations can be easily obtained from commercial supply sources.

### Reference Example 1

### 3-Methoxyazetidine hydrochloride

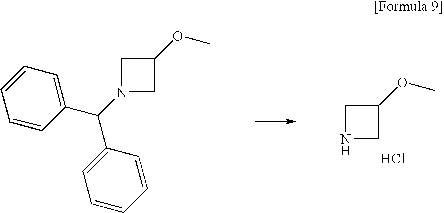

1-Benzhydryl-3-methoxyazetidine (J. Org. Chem., 1972, 37, 3953, 1.10 g) was dissolved in ethanol (40 ml), the solution was added with 20% palladium hydroxide/carbon (1.1 g), and catalytic reduction was performed for 17 hours under a hydrogen atmosphere. The catalyst was removed by filtration, and the reaction mixture was added with 1 N hydrochloric acid in ethanol (4.5 ml), concentrated, and then added with ether. The deposited solid was collected by filtration, and dried to obtain the title compound (480 mg) as colorless solid.

¹H-NMR (400 MHz, d₆-DMSO) δ: 2.17 (3H, s), 3.75-3.79 (2H, m), 4.06-4.11 (2H, m), 4.21-4.27 (1H, m), 9.28 (2H, br).

ESI-MS; m/z: 88 (M+H)⁺.

### Reference Example 2

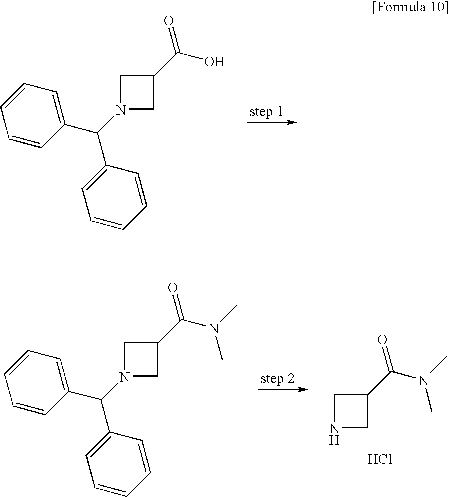

### Step 1: 1-Benzhydryl-N,N-dimethyl-3-azetidinecarboxamide

1-Benzhydryl-3-azetidinecarboxylic acid (1.17 g), dimethylamine (2 mole solution in tetrahydrofuran, 2.6 ml), triethylamine (0.67 ml) and 1-hydroxybenzotriazole (300 mg) were dissolved in dichloromethane (25 ml) and cooled on ice. Then, the solution was added with 1-ethyl-3-(3-dimethylaminopropyl)carbodiimide hydrochloride (925 mg), and the mixture was returned to room temperature, and stirred for 6 hours. The reaction mixture was added with saturated aqueous sodium hydrogencarbonate, and extracted with ethyl acetate. The organic layer was successively washed with water and saturated brine, and dried over anhydrous sodium sulfate, and the solvent was evaporated under reduced pressure. The resulting residue was purified by silica gel chromatography [developing solvent; ethyl acetate:n-hexane=2:1 (v/v)] to obtain the title compound (1.10 g) as colorless solid.

¹H-NMR (400 MHz, CDCl₃) δ: 2.85 (3H, s), 2.92 (3H, s), 3.23-3.29 (2H, m), 3.46-3.48 (3H, m), 4.38 (1H, s), 7.16-7.41 (10H, m).

### Step 2: N,N-Dimethyl-3-azetidinecarboxamide hydrochloride

The compound obtained in Step 1 mentioned above was used as a starting material to perform the same procedure as that of Reference Example 1 and thereby to obtain the title compound as colorless solid.

¹H-NMR (400 MHz, d₆-DMSO) δ: 2.82 (3H, s), 2.84 (3H, s), 3.85-3.94 (1H, m), 4.03-4.05 (4H, m), 9.10 (1H, br), 9.60 (1H, br).

ESI-MS; m/z: 129 (M+H)⁺.

### Reference Example 3

### 3-Methyl-3-azetidinol hydrochloride

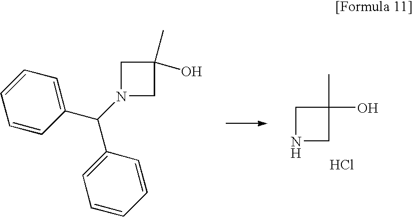

1-Benzhydryl-3-methyl-3-azetidinol (Tetrahedron Lett., 1996, 37, 1297) was used as a starting material to perform the same procedure as that of Reference Example 1 and thereby to obtain the title compound as colorless solid.

¹H-NMR (400 MHz, d₆-DMSO) δ: 1.43 (3H, s), 3.71 (2H, d, J=11.2 Hz), 3.83 (2H, d, J=11.2 Hz), 6.11 (1H, s), 9.38 (2H, br).

ESI-MS; m/z: 88 (M+H)⁺.

### Reference Example 4

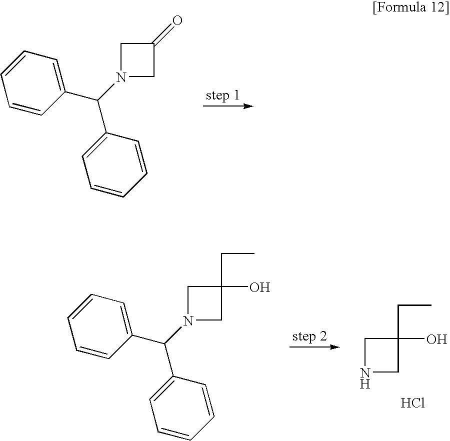

### Step 1: 1-Benzhydryl-3-ethyl-3-azetidinol

A solution of ethyl lithium [0.5 mole solution in benzene:cyclohexane (90:10), 40 ml] was added dropwise with an ether solution (6 ml) of 1-benzhydrylazetidin-3-one (J. Heterocycl. Chem., 1994, 31, 271, 2.37 g) at room temperature under a nitrogen atmosphere, and the mixture was refluxed for 0.5 hour by heating. The reaction mixture was cooled on ice, and then added with water, and the mixture was stirred for about 5 minutes. The reaction mixture was added with ether, the organic layer was dried over anhydrous sodium sulfate, and the solvent was evaporated under reduced pressure. The resulting residue was purified by silica gel chromatography [developing solvent; ethyl acetate:n-hexane=1:4 (v/v)] to obtain the title compound (2.67 g) as colorless oil.

¹H-NMR (400 MHz, CDCl₃) δ: 0.96 (3H, t, J=7.3 Hz), 1.80 (2H, q, J=7.3 Hz), 1.86 (1H, br), 2.94 (2H, d, J=8.0 Hz), 3.21 (2H, d, J=8.0 Hz), 4.12 (1H, d, J=7.0 Hz), 7.15-7.42 (10H, m).

### Step 2: 3-Ethyl-3-azetidinol hydrochloride

The compound obtained in Step 1 mentioned above was used as a starting material to perform the same procedure as that of Reference Example 1 and thereby to obtain the title compound as colorless solid.

¹H-NMR (400 MHz, d₆-DMSO) δ: 0.84 (3H, t, J=7.3 Hz), 1.75 (2H, q, J=7.3 Hz), 3.72-3.74 (2H, m), 3.77-3.81 (2H, m), 6.04 (1H, brs), 9.22 (1H, br), 9.47 (1H, br).

ESI-MS; m/z: 102 (M+H)⁺.

### Reference Example 5

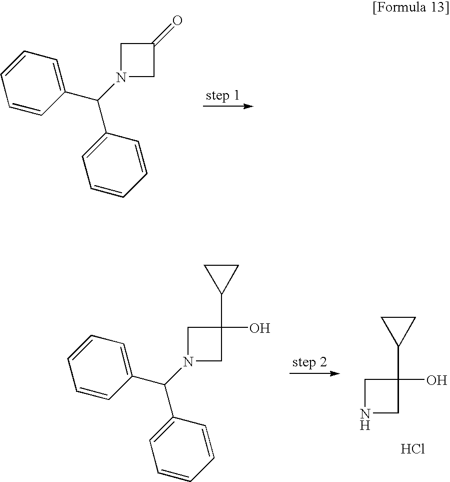

### Step 1: 1-Benzhydryl-3-cyclopropyl-3-azetidinol

Metal magnesium (1.03 g) was suspended in tetrahydrofuran (30 ml), the suspension was added with a catalytic amount of iodine (20 mg), and the atmosphere was purged with nitrogen. Then, the reaction mixture was slowly added dropwise with bromocyclopropane (3.50 ml), and then further stirred for 30 minutes. The reaction mixture was cooled to −78° C., then added dropwise with a solution of 1-benzhydrylazetidin-3-one (2.00 g) in tetrahydrofuran (10 ml), and stirred for 1 hour. The reaction mixture was cooled on ice, then added with saturated aqueous sodium hydrogencarbonate, and extracted with ethyl acetate. The organic layer was successively washed with water and saturated brine, and then dried over anhydrous sodium sulfate, and the solvent was evaporated under reduced pressure. The resulting residue was purified by silica gel chromatography [developing solvent; ethyl acetate:n-hexane=1:2 (v/v)] to obtain the title compound (2.20 g) as colorless oil.

¹H-NMR (400 MHz, CDCl₃) δ: 0.45-0.51 (2H, m), 0.52-0.58 (2H, m), 1.17-1.27 (1H, m), 1.98 (1H, br), 2.92 (2H, d, J=8.8 Hz), 3.09 (2H, d, J=8.8 Hz), 4.34 (1H, s), 7.15-7.41 (10H, m).

### Step 2: 3-Cyclopropyl-3-azetidinol hydrochloride

The compound obtained in Step 1 mentioned above was used as a starting material to perform the same procedure as that of Reference Example 1 and thereby to obtain the title compound as colorless solid.

¹H-NMR (400 MHz, d₆-DMSO) δ: 0.41 (4H, d, J=6.8 Hz), 1.25-1.32 (1H, m), 3.70-3.81 (4H, m), 6.04 (1H, br), 9.31 (2H, br).

ESI-MS; m/z: 114 (M+H)⁺.

### Reference Example 6

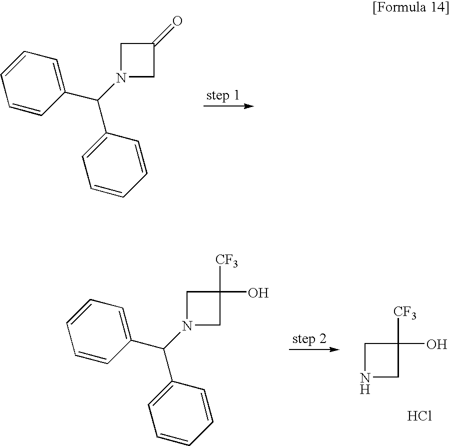

### Step 1: 1-Benzhydryl-3-trifluoromethyl-3-azetidinol

A solution of 1-benzhydrylazetidin-3-one (2.00 g) in tetrahydrofuran (10 ml) was cooled on ice, added with (trifluoromethyl)trimethylsilane (1.50 ml) under a nitrogen atmosphere, and added with tetra-n-butylammonium fluoride (1 mole solution in tetrahydrofuran, 0.10 ml). After 10 minutes, the reaction mixture was added dropwise with tetra-n-butylammonium fluoride (1 mole solution in tetrahydrofuran, 8.50 ml), returned to room temperature, and stirred for 15 minutes. The reaction mixture was diluted with ethyl acetate, the organic layer was successively washed with water (2 times) and saturated brine, and then dried over anhydrous sodium sulfate, and the solvent was evaporated under reduced pressure. The resulting residue was purified by silica gel chromatography [developing solvent; ethyl acetate:n-hexane=1:4 (v/v)] to obtain the title compound (2.07 g) as pale yellow oil.

¹H-NMR (400 MHz, CDCl₃) δ: 2.66 (1H, br), 3.19 (2H, d, J=9.3 Hz), 3.53 (2H, d, J=9.3 Hz), 4.45 (1H, s), 7.17-7.43 (10H, m).

### Step 2: 3-Trifluoromethyl-3-azetidinol hydrochloride

The compound obtained in Step 1 mentioned above was used as a starting material to perform the same procedure as that of Reference Example 1 and thereby to obtain the title compound as colorless solid.

¹H-NMR (400 MHz, d₆-DMSO) δ: 4.05 (2H, d, J=1.5 Hz), 4.08 (2H, d, J=1.5 Hz), 7.99 (1H, s), 9.80 (2H, br).

ESI-MS; m/z: 142 (M+H)⁺.

### Reference Example 7

### 3-Azetidinecarbonitrile hydrochloride

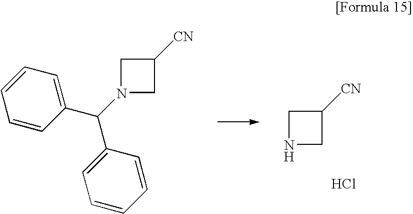

1,2-Dichloroethane (16 ml) was added with 1-benzhydrylazetidine-3-carbonitrile (1.24 g), and added with 1-chloroethyl chloroformate (0.82 ml) at room temperature, and the mixture was refluxed for 0.5 hour by heating. The reaction mixture was concentrated, then the resulting residue was added with methanol (16 ml), and the mixture was refluxed for 1 hour by heating, and left to cool. The mixture was concentrated again, and then the resulting residue was added with ethyl acetate, and the deposited solid was collected by filtration, and dried to obtain the title compound (360 mg) as colorless solid.

¹H-NMR (400 MHz, d₆-DMSO) δ: 3.94-4.02 (1H, m), 4.14 (2H, s), 4.16 (2H, s), 9.61 (2H, br).

EI-MS; m/z: 82 (M⁺).

### Reference Example 8

### 3-Azetidinemethanol hydrochloride

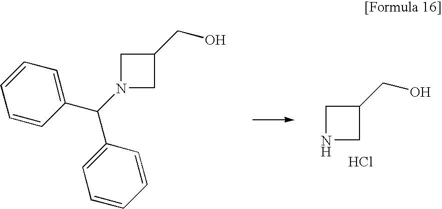

(1-Benzhydryl-3-azetidino)methanol was used as a starting material to perform the same procedure as that of Reference Example 1 and thereby to obtain the title compound as colorless solid.

¹H-NMR (400 MHz, d₆-DMSO) δ: 2.78-2.87 (1H, m), 3.51 (2H, t, J=5.4 Hz), 3.70-3.74 (2H, m), 3.90 (2H, t, J=9.5 Hz), 5.69 (1H, t, J=5.4 Hz), 9.13 (2H, br).

ESI-MS; m/z: 88 (M+H)⁺.

### Reference Example 9

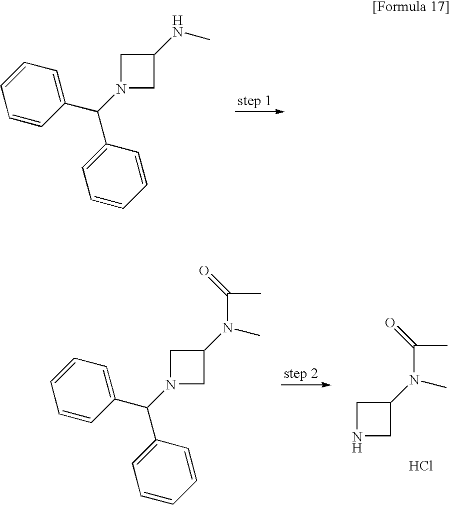

### Step 1: N-(1-Benzhydryl-3-azetidino)-N-methylacetamide

1-Benzhydryl-3-azetidinomethylamine (J. Med. Chem., 1993, 36, 801, 1.50 g) and triethylamine (0.92 ml) were dissolved in dichloromethane (30 ml), and the solution was added with acetic anhydride (0.62 ml), and stirred at room temperature for 20 hours. The reaction mixture was concentrated, and then the resulting residue was diluted with ethyl acetate. The organic layer was successively washed with water and saturated brine, and then dried over anhydrous sodium sulfate, and the solvent was evaporated under reduced pressure. The resulting residue was added with ether, and the deposited solid was collected by filtration, and dried to obtain the title compound (1.42 g) as colorless solid.

¹H-NMR (400 MHz, CDCl₃) δ: 2.03, 2.07 (total 3H, each s), 3.02, 3.11 (total 3H, each s), 3.05, 3.13 (total 2H, each t, J=7.3 Hz), 3.47 (2H, t, J=7.0 Hz), 4.34, 4.35 (total 1H, each s), 4.45-4.52, 4.87-4.93 (total 1H, each m), 7.16-7.41 (10H, m).

### Step 2: N-(3-Azetidino)-N-methylacetamide hydrochloride

The compound obtained in Step 1 mentioned above was used as a starting material to perform the same procedure as that of Reference Example 1 and thereby to obtain the title compound as colorless solid.

¹H-NMR (400 MHz, d₆-DMSO) δ: 2.01 (3H, s), 3.00 (3H, s), 3.97-4.15 (4H, m), 4.94-5.02 (1H, m), 9.21 (2H, br).

ESI-MS; m/z: 129 (M+H)⁺.

### Reference Example 10

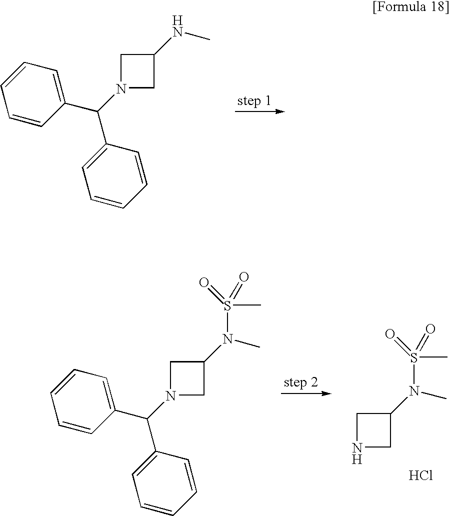

### Step 1: N-(1-Benzhydryl-3-azetidino)-N-methylmethanesulfonamide

1-Benzhydryl-3-azetidinomethylamine (1.50 g) and triethylamine (0.92 ml) were dissolved in dichloromethane (30 ml), and the solution was cooled on ice. Then, the solution was added with methanesulfonyl chloride (0.51 ml), and stirred for 1 hour. The reaction mixture was concentrated, and then the resulting residue was diluted with ethyl acetate. The organic layer was successively washed with water and saturated brine, and then dried over anhydrous sodium sulfate, and the solvent was evaporated under reduced pressure. The resulting residue was added with ether, and the deposited solid was collected by filtration, and dried to obtain the title compound (1.55 g) as colorless solid.

¹H-NMR (400 MHz, CDCl₃) δ: 2.72 (3H, s), 2.90 (3H, s), 3.16 (2H, br), 3.49 (2H, br), 4.23-4.37 (1H, m), 4.37 (1H, br), 7.18-7.41 (10H, m).

### Step 2: N-(3-Azetidino)-N-methylmethanesulfonamide hydrochloride

The compound obtained in Step 1 mentioned above was used as a starting material to perform the same procedure as that of Reference Example 7 and thereby to obtain the title compound as colorless solid.

¹H-NMR (400 MHz, d₆-DMSO) δ: 2.87 (3H, s), 2.93 (3H, s), 4.04-4.12 (2H, m), 4.15-4.21 (2H, m), 4.60-4.69 (1H, m), 9.23 (2H, br).

ESI-MS; m/z: 165 (M+H)⁺.

### Reference Example 11

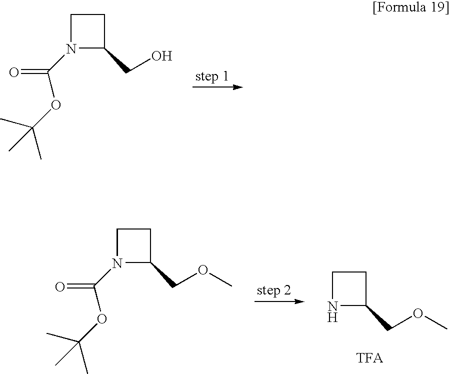

### Step 1: (S)-2-(Methoxymethyl)-1-azetidinecarboxylic acid tert-butyl ester

60% Sodium hydride (240 mg) was suspended in N,N-dimethylformamide (12 ml), and the suspension was cooled on ice under a nitrogen atmosphere. The suspension was added dropwise with a solution of (2S)-2-(hydroxymethyl)-1-azetidinecarboxylic acid tert-butyl ester (936 mg) dissolved in N,N-dimethylformamide (6 ml), and then stirred for 20 minutes. Then, the reaction mixture was added with methyl iodide (0.40 ml), returned to room temperature, and stirred overnight. The reaction mixture was added with cooled saturated aqueous ammonium chloride, and extracted with ethyl acetate. The organic layer was washed with saturated brine, and dried over anhydrous sodium sulfate, and then the solvent was evaporated under reduced pressure. The resulting residue was purified by silica gel chromatography [developing solvent; ethyl acetate:n-hexane=1:4 (v/v)] to obtain the title compound (910 mg) as colorless oil.

¹H-NMR (400 MHz, CDCl₃) δ: 1.44 (9H, s), 2.11-2.27 (2H, m), 3.41 (3H, s), 3.54 (1H, dd, J=10, 3.2 Hz), 3.65 (1H, dd, J=10, 5.4 Hz), 3.81 (2H, dd, J=8.2, 6.9 Hz), 4.27-4.33 (1H, m).

### Step 2: (S)-2-(Methoxymethyl)azetidine trifluoroacetate

The compound obtained in Step 1 mentioned above (905 mg) was dissolved in dichloromethane (4 ml), and the solution was added with trifluoroacetic acid (TFA, 4 ml) under ice cooling, returned to room temperature, and stirred for 1 hour. The reaction mixture was concentrated to obtain the title compound (970 mg) as colorless oil.

¹H-NMR (400 MHz, d₆-DMSO) δ: 2.21-2.30 (1H, m), 2.34-2.40 (1H, m), 3.34 (3H, s), 3.53 (1H, dd, J=11.3, 3.5 Hz), 3.65 (1H, dd, J=11.3, 6.9 Hz), 3.70-3.77 (1H, m), 3.84-3.93 (1H, m), 4.50 (1H, br), 8.72 (2H, br).

ESI-MS; m/z: 102 (M+H)⁺.

### Reference Example 12

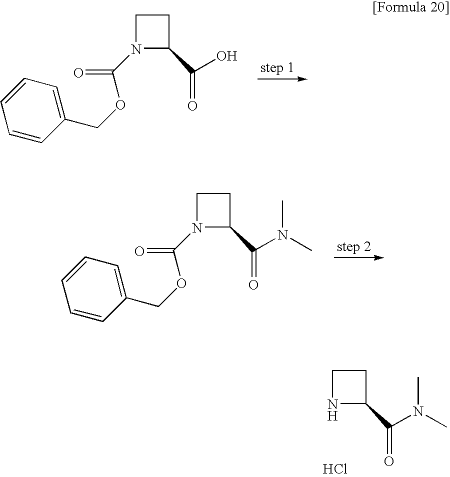

### Step 1: (2S)-2-(Dimethylcarbamoyl)-1-azetidinecarboxylic acid benzyl ester

(2S)-1-[(Benzyloxy)carbonyl]-2-azetidinecarboxylic acid (Tetrahedron, 1993, 49, 8211) was used as a starting material to perform the same procedure as that of Reference Example 2, Step 1 and thereby to obtain the title compound as colorless oil.

¹H-NMR (400 MHz, CDCl₃) δ: 2.20-2.27 (1H, m), 2.45-2.54 (1H, m), 2.97 (6H, brs), 3.92-3.98 (1H, m), 4.11-4.17 (1H, m), 5.00-5.03 (1H, m), 5.09 (2H, brs), 7.27-7.35 (5H, m).

### Step 2: (2S)—N,N-Dimethyl-2-azetidinecarboxamide hydrochloride

The compound obtained in Step 1 mentioned above was used as a starting material to perform the same procedure as that of Reference Example 1 and thereby to obtain the title compound as colorless solid.

¹H-NMR (400 MHz, d₆-DMSO) δ: 2.39-2.50 (1H, m), 2.70-2.79 (1H, m), 2.83 (3H, s), 2.90 (3H, s), 3.66-3.72 (1H, m), 3.88-3.93 (1H, m), 5.27 (1H, t, J=8.5 Hz), 8.68 (1H, br), 10.29 (1H, br).

ESI-MS; m/z: 129 (M+H)⁺.

### Reference Example 13

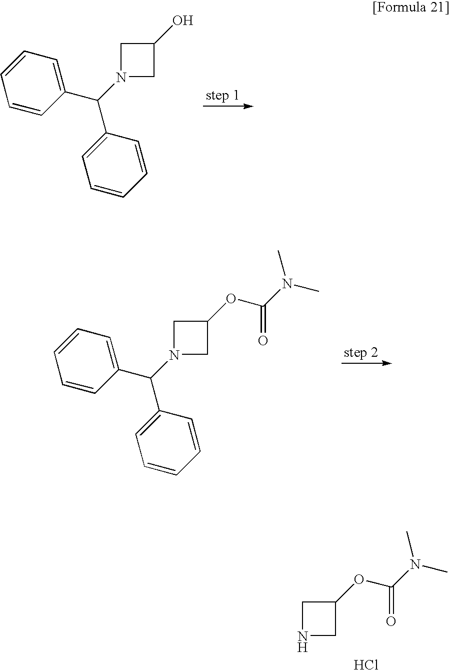

### Step 1: 1-Benzhydryl-3-azetidino dimethylcarbamate

60% Sodium hydride (480 mg) was suspended in N,N-dimethylformamide (5 ml), and the suspension was cooled on ice. The suspension was added dropwise with a solution of 1-benzhydrylazetidin-3-ol (2.39 g) dissolved in tetrahydrofuran (20 ml), and then the mixture was stirred for 30 minutes. Then, the mixture was added with N,N-dimethylcarbamoyl chloride (1.1 ml), returned to room temperature, and stirred overnight. The reaction mixture was added with cooled saturated aqueous ammonium chloride, and extracted with ethyl acetate. The organic layer was washed with saturated aqueous sodium hydrogencarbonate and saturated brine, and dried over anhydrous sodium sulfate, and then the solvent was evaporated under reduced pressure. The resulting residue was added with ether/n-hexane, and the deposited solid was collected by filtration, and dried to obtain the title compound (2.40 g) as colorless solid.

¹H-NMR (400 MHz, CDCl₃) δ: 2.88 (3H, brs), 2.90 (3H, brs), 2.98-3.02 (2H, m), 3.58-3.62 (2H, m), 4.37 (1H, s), 5.00-5.06 (1H, m), 7.16-7.29 (10H, m).

### Step 2: 3-Azetidino dimethylcarbamate hydrochloride

The compound obtained in Step 1 mentioned above was used as a starting material to perform the same procedure as that of Reference Example 1 and thereby to obtain the title compound as colorless solid.

¹H-NMR (400 MHz, d₆-DMSO) δ: 2.82 (3H, s), 2.90 (3H, s), 3.92-3.95 (2H, m), 4.17-4.22 (2H, m), 5.04-5.09 (1H, m), 9.37 (1H, br).

ESI-MS; m/z: 145 (M+H)⁺.

### Reference Example 14

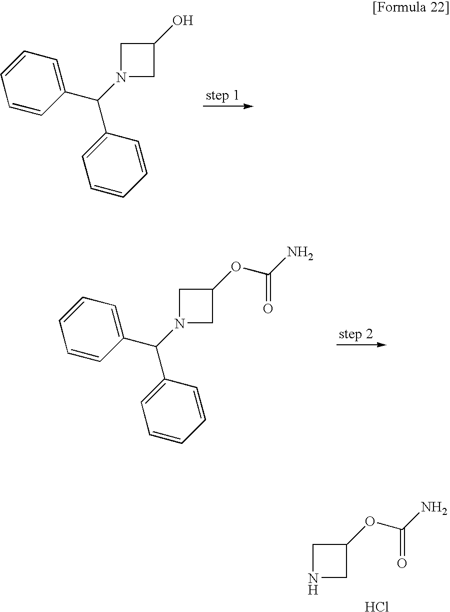

### Step 1: 1-Benzhydryl-3-azetidino carbamate

1-Benzhydrylazetidin-3-ol (2.39 g) was dissolved in ethyl acetate (50 ml) and cooled on ice. Then, the solution was added with trichloroacetyl isocyanate (1.25 ml), and stirred at the same temperature for 1 hour, and then the reaction mixture was concentrated under reduced pressure. The resulting residue was dissolved in methanol (50 ml), and the solution was added with water (4 ml) and sodium formate (816 mg), and stirred for 24 hours at room temperature. The reaction mixture was concentrated, and then diluted with ethyl acetate. The reaction mixture was washed with saturated aqueous sodium hydrogencarbonate and saturated brine, and dried over anhydrous sodium sulfate, and then the solvent was evaporated under reduced pressure. The resulting residue was added with ether, and the deposited solid was collected by filtration, and dried to obtain the title compound (1.75 g) as colorless solid.

¹H-NMR (400 MHz, CDCl₃) δ: 3.05 (2H, t, J=8.0 Hz), 3.57 (2H, t, J=8.0 Hz), 4.37 (1H, s), 4.61 (2H, br), 5.01-5.08 (1H, m), 7.16-7.40 (10H, m).

### Step 2: 3-Azetidino carbamate hydrochloride

The compound obtained in Step 1 mentioned above was used as a starting material to perform the same procedure as that of Reference Example 1 and thereby to obtain the title compound as colorless solid.

¹H-NMR (400 MHz, d₆-DMSO) δ: 3.86-3.90 (2H, m), 4.16-4.21 (2H, m), 5.01-5.06 (1H, m), 6.77 (1H, br), 6.93 (1H, br), 9.23 (2H, br).

ESI-MS; m/z: 117 (M+H)⁺.

### Reference Example 15

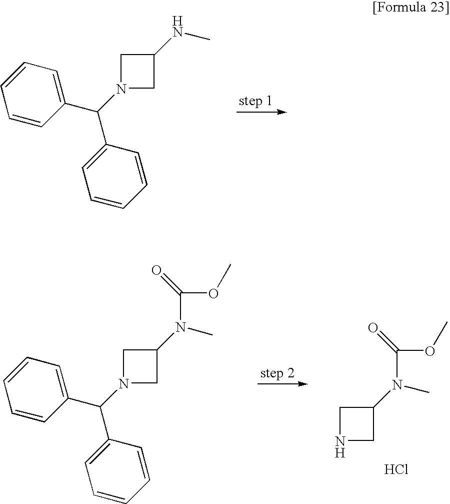

### Step 1: Methyl 1-benzhydryl-3-azetidino(methyl)carbamate

1-Benzhydryl-3-azetidinomethylamine (1.50 g) was dissolved in dichloromethane (25 ml), and the solution was added with triethylamine (1.0 ml) and cooled on ice. Then, the reaction mixture was added dropwise with methyl chloroformate (0.53 ml), returned to room temperature, and stirred overnight. The reaction mixture was concentrated, and then the resulting residue was diluted with ethyl acetate. The organic layer was successively washed with water, saturated aqueous sodium hydrogencarbonate and saturated brine, and then dried over anhydrous sodium sulfate, and the solvent was evaporated under reduced pressure. The resulting residue was added with ether, and the deposited solid was collected by filtration, and dried to obtain the title compound (1.52 g) as colorless solid.

¹H-NMR (400 MHz, CDCl₃) δ: 2.95 (3H, s), 3.02 (2H, brs), 3.42-3.46 (2H, m), 3.65 (3H, s), 4.32 (1H, s), 4.49 (1H, br), 7.16-7.41 (10H, m).

### Step 2: Methyl 3-azetidino(methyl)carbamate hydrochloride

The compound obtained in Step 1 mentioned above was used as a starting material to perform the same procedure as that of Reference Example 1 and thereby to obtain the title compound as colorless solid.

¹H-NMR (400 MHz, d₆-DMSO) δ: 2.90 (3H, s), 3.62 (3H, s), 4.01 (2H, brs), 4.13 (2H, brs), 4.81-4.87 (1H, m), 9.12 (2H, br).

ESI-MS; m/z: 145 (M+H)⁺.

### Reference Example 16

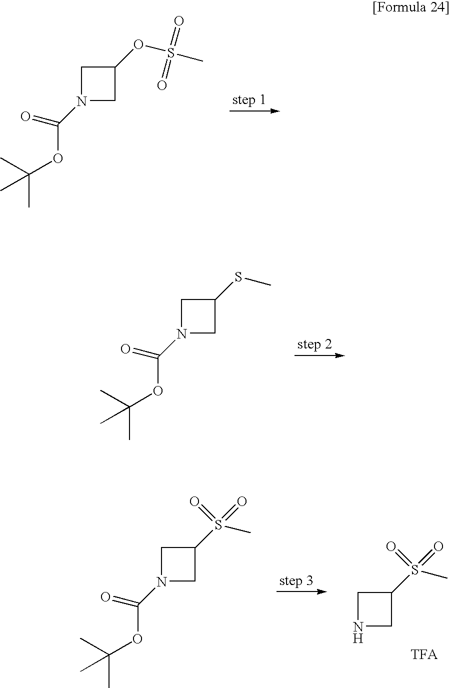

### Step 1: 3-(Methylsulfanyl)-1-azetidinecarboxylic acid tert-butyl ester

3-[(Methanesulfonyl)oxy]-1-azetidinecarboxylic acid tert-butyl ester (J. Med. Chem., 2001, 44, 94, 2.60 g) was dissolved in N,N-dimethylformamide (16 ml), and the solution was added with sodium thiomethoxide (1.45 g), and stirred at 80° C. for 20 hours under a nitrogen atmosphere. The reaction mixture was left to cool, then poured into ice water, and extracted with ethyl acetate. The organic layer was successively washed with water and saturated brine, and then dried over anhydrous sodium sulfate, and the solvent was evaporated under reduced pressure. The resulting residue was purified by silica gel chromatography [developing solvent; ethyl acetate:n-hexane=1:4 (v/v)] to obtain the title compound (2.04 g) as colorless oil.

¹H-NMR (400 MHz, CDCl₃) δ: 1.44 (9H, s), 2.11 (3H, s), 3.53-3.60 (1H, m), 3.82 (2H, dd, J=9.0, 5.5 Hz), 4.23 (2H, dd, J=9.0, 8.1 Hz).

### Step 2: 3-(Methanesulfonyl)-1-azetidinecarboxylic acid tert-butyl ester

The compound obtained in Step 1 mentioned above (2.04 g) was dissolved in methanol (20 ml), and the solution was cooled on ice. Then, the solution was slowly added with a solution of oxone (12.3 g) dissolved in water (12.4 ml), and stirred for 4 hours at the same temperature. The reaction mixture was diluted with water, and then extracted 4 times with ethyl acetate. The organic layer was dried over anhydrous sodium sulfate, and the solvent was evaporated under reduced pressure. The resulting residue was added with ethyl acetate/n-hexane, and the deposited solid was collected by filtration, and dried to obtain the title compound (1.85 g) as colorless solid.

¹H-NMR (400 MHz, CDCl₃) δ: 1.45 (9H, s), 2.90 (3H, s), 3.86-3.93 (1H, m), 4.19-4.29 (4H, m).

### Step 3: 3-(Methanesulfonyl)azetidine trifluoroacetate

The compound obtained in Step 1 mentioned above was used as a starting material to perform the same procedure as that of Reference Example 12, Step 2 and thereby to obtain the title compound as colorless solid.

¹H-NMR (400 MHz, d₆-DMSO) δ: 3.09 (3H, s), 4.15-4.20 (2H, m), 4.27-4.33 (2H, m), 4.49-4.57 (1H, m), 9.41 (2H, br).

ESI-MS; m/z: 136 (M+H)⁺.

### Reference Example 17

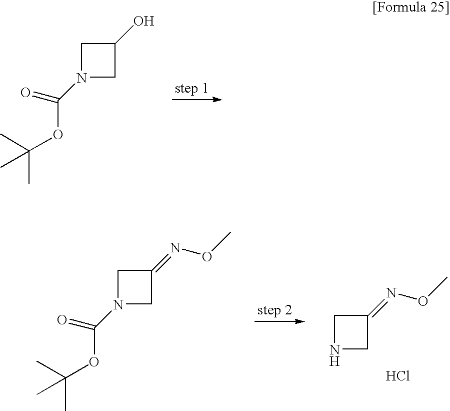

### Step 1: 3-(Methoxyimino)-1-azetidinecarboxylic acid tert-butyl ester

3-Hydroxy-1-azetidinecarboxylic acid tert-butyl ester (1.50 g) was dissolved in dichloromethane (15 ml), and the solution was added dropwise with a solution of Dess-Martin reagent (5.51 g) in dichloromethane (60 ml), and then further stirred for 3 hours. The reaction mixture was added with saturated aqueous sodium hydrogencarbonate and sodium thiosulfate pentahydrate (10 g), and vigorously stirred for about 30 minutes. The organic layer was successively washed with water and saturated brine, dried over anhydrous sodium sulfate, and the solvent was evaporated under reduced pressure. The resulting residue was dissolved in a mixture of ethanol (30 ml) and water (15 ml), and the solution was added with methoxyamine hydrochloride (1.08 g) and sodium acetate (1.06 g), and refluxed for 1 hour by heating. The reaction mixture was left to cool, and then concentrated, and the resulting residue was added with water, and extracted with ethyl acetate. The organic layer was successively washed with saturated aqueous sodium hydrogencarbonate and saturated brine, and dried over anhydrous sodium sulfate, and the solvent was evaporated under reduced pressure. The resulting residue was purified by silica gel chromatography [developing solvent; ethyl acetate:n-hexane=1:4 (v/v)] to obtain the title compound (1.60 g) as colorless solid.

¹H-NMR (400 MHz, CDCl₃) δ: 1.46 (9H, s), 3.87 (3H, s), 4.57-4.60 (4H, m).

### Step 2: 3-Azetidine O-methyloxime hydrochloride

The compound obtained in Step 1 mentioned above (1.60 g) was dissolved in methanol (60 ml), and the solution was added with a 4 N hydrochloric acid solution in dioxane (10 ml), and stirred at room temperature for 15 hours. The reaction mixture was concentrated, and then added with tetrahydrofuran, and the deposited solid was collected by filtration, and dried to obtain the title compound (960 mg) as colorless solid (hygroscopic).

¹H-NMR (400 MHz, d₆-DMSO) δ: 3.82 (3H, t, J=1.2 Hz), 4.73 (2H, d, J=1.2 Hz), 4.79 (2H, s), 9.84 (2H, br).

ESI-MS; m/z: 101 (M+H)⁺.

### Reference Example 18

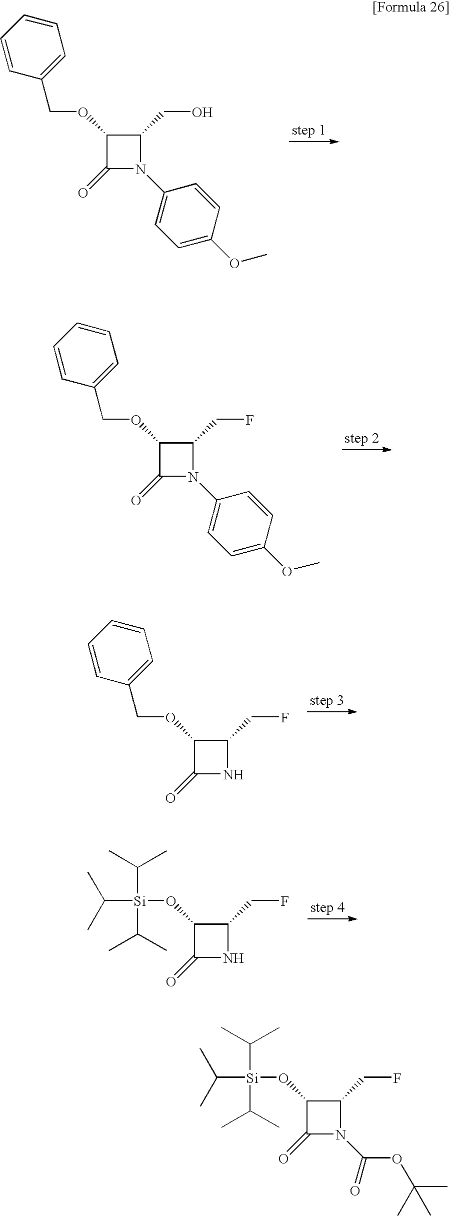

### Step 1: (3R,4R)-3-(Benzyloxy)-4-(fluoromethyl)-1-(4-methoxyphenyl)-2-azetidinone

(3R,4S)-3-(Benzyloxy)-4-(hydroxymethyl)-1-(4-methoxyphenyl)-2-azetidinone (J. Med. Chem., 1994, 37, 2655, 1.00 g) was dissolved in dichloromethane (16 ml), and the solution was added with diethylaminosulfur trifluoride (0.843 ml) at −78° C., and stirred for 23 hours with warming to room temperature. The reaction mixture was cooled to 0° C., and then added with methanol. The solvent was evaporated under reduced pressure, and the resulting residue was purified by silica gel chromatography [developing solvent; ethyl acetate:n-hexane=1:9 (v/v)] to obtain the title compound (333 mg) as colorless solid.

¹H-NMR (400 MHz, CDCl₃) δ: 3.79 (3H, s), 4.41-4.48 (1H, m), 4.66-4.91 (5H, m), 6.85-6.89 (2H, m), 7.31-7.46 (7H, m).

ESI-MS; m/z: 316 (M+H)⁺.

### Step 2: (3R,4R)-3-(Benzyloxy)-4-(fluoromethyl)-2-azetidinone

The compound obtained in Step 1 mentioned above (897 mg) was dissolved in acetonitrile (15 ml) and water (2 ml), and the solution was added with an aqueous solution (13 ml) of cerium(IV) diammonium nitrate (5.46 g) at 0° C., and stirred for 1 hour. The reaction mixture was added with ethyl acetate, and the organic layer was washed with water, saturated aqueous sodium hydrogencarbonate, and saturated brine, and dried over anhydrous sodium sulfate. Then, the solvent was evaporated under reduced pressure, and the resulting residue was purified by silica gel chromatography [developing solvent; ethyl acetate:n-hexane=1:3 to 1:1 (v/v)] to obtain the title compound (408 mg) as yellow solid.

¹H-NMR (400 MHz, CDCl₃) δ: 4.03 (1H, ddt, J=11.2, 7.6, 4.6 Hz), 4.47-4.69 (3H, m), 4.80-4.84 (2H, m), 6.23 (1H, br s), 7.26-7.39 (5H, m).

ESI-MS; m/z: 210 (M+H)⁺.

### Step 3: (3R,4R)-4-(Fluoromethyl)-3-[(triisopropylsilyl)oxy]-2-azetidinone

The compound obtained in Step 2 mentioned above (408 mg) was dissolved in ethyl acetate (10 ml), and the solution was added with 5% palladium/carbon (408 mg) at room temperature, and stirred for 3 days under a hydrogen atmosphere. The reaction mixture was filtered through Celite, and the filtrate was concentrated under reduced pressure. The residue was dissolved in N,N-dimethylformamide (4 ml), and the solution was added with imidazole (199 mg) and triisopropylsilyl chloride (0.645 ml) at 0° C., and stirred at room temperature for 18 hours. The reaction mixture was added with water, and extracted with ethyl acetate, and the organic layer was washed with water and saturated brine. The organic layer was dried over anhydrous sodium sulfate, and then the solvent was evaporated under reduced pressure. The resulting residue was purified by silica gel chromatography [developing solvent; ethyl acetate:n-hexane=1:3 (v/v)] to obtain the title compound (432 mg) as colorless oil.

¹H-NMR (400 MHz, CDCl₃) δ: 1.07-1.28 (21H, m), 4.00-4.07 (1H, m), 4.57 (1H, ddd, J=48.1, 10.0, 8.1 Hz), 4.73 (1H, ddd, J=46.4, 10.0, 3.9 Hz), 5.09 (1H, dd, J=5.1, 2.4 Hz), 6.11 (1H, br s).

### Step 4: (3R,4R)-1-(tert-Butoxycarbonyl)-4-(fluoromethyl)-3-[(triisopropylsilyl)oxy]-2-azetidinone

The compound obtained in Step 3 mentioned above (432 mg) and di-tert-butyl dicarbonate (529 mg) were dissolved in tetrahydrofuran (16 ml), and the solution was added with 4-dimethylaminopyridine (38.3 mg) at room temperature, and stirred for 16.5 hours. The reaction mixture was concentrated under reduced pressure, and the resulting residue was purified by silica gel chromatography [developing solvent; ethyl acetate:n-hexane=3:97 (v/v)] to obtain the title compound (555 mg) as colorless oil.

¹H-NMR (400 MHz, CDCl₃) δ: 0.91-1.28 (21H, m), 1.52 (9H, s), 4.27 (1H, ddt, J=15.4, 5.6, 3.9 Hz), 4.73 (1H, ddd, J=45.9, 9.8, 3.9 Hz), 4.81 (1H, ddd, J=46.9, 9.8, 5.6 Hz), 5.07 (1H, d, J=5.6 Hz).

### Reference Example 19

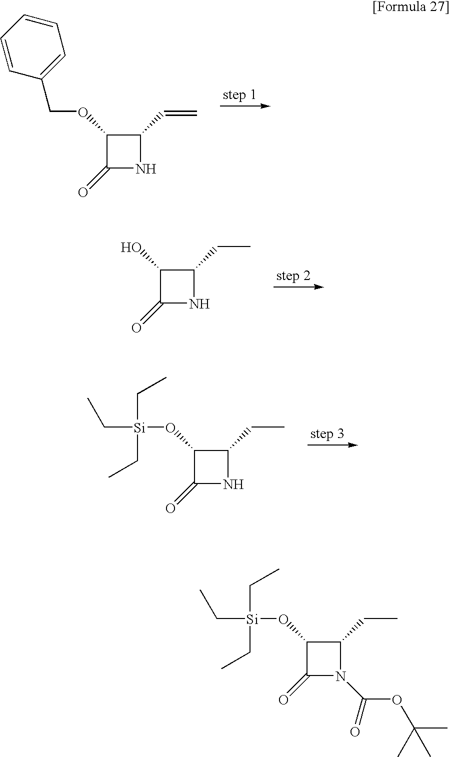

### Step 1: (3R,4S)-4-Ethyl-3-hydroxy-2-azetidinone

(3R,4S)-3-(Benzyloxy)-4-vinyl-2-azetidinone (J. Med. Chem., 1994, 37, 2655, 680 mg) was dissolved in methanol (10 ml), and the solution was added with 5% palladium/carbon (680 mg×2) at room temperature, and stirred for 44 hours under a hydrogen atmosphere. The catalyst was removed by filtration through Celite, and the filtrate was concentrated to obtain the title compound (450 mg) as red oil.

¹H-NMR (DMSO-d₆) δ: 0.87 (3H, t, J=7.4 Hz), 1.34-1.57 (2H, m), 3.36-3.42 (1H, m), 4.65 (1H, d, J=4.6 Hz), 5.86 (1H, brs), 8.17 (1H, brs).

### Step 2: (3R,4S)-4-Ethyl-3-[(triethylsilyl)oxy]-2-azetidinone

The compound obtained in Step 1 mentioned above (580 mg) was dissolved in N,N-dimethylformamide (5 ml), and the solution was added with imidazole (1.04 g). The mixture was added dropwise with triethylsilyl chloride (1.28 ml) at 0° C., returned to room temperature, and stirred for 14 hours. The reaction mixture was added with water, and extracted with ethyl acetate, and the organic layer was washed with saturated aqueous sodium hydrogencarbonate and saturated brine, and dried over anhydrous sodium sulfate. Then, the solvent was evaporated under reduced pressure, and the resulting residue was purified by silica gel chromatography [developing solvent; ethyl acetate:n-hexane=1:6 (v/v)] to obtain the title compound (920 mg) as pale yellow amorphous solid.

¹H-NMR (CDCl₃) δ: 0.63-0.73 (6H, m), 0.93-1.01 (12H, m), 1.55-1.73 (2H, m), 3.57-3.63 (1H, m), 4.85 (1H, dd, J=4.6, 2.4 Hz), 5.95 (1H, brs).

### Step 3: (3R,4S)-1-(tert-Butoxycarbonyl)-4-ethyl-3-[(triethylsilyl)oxy]-2-azetidinone

The compound obtained in Step 2 mentioned above was used as a starting material to perform the same procedure as that of Reference Example 18, Step 4 and thereby to obtain the title compound as pale yellow amorphous solid.

¹H-NMR (CDCl₃) δ: 0.63-0.73 (6H, m), 0.96-1.00 (12H, m), 1.51 (9H, s), 1.75-1.94 (2H, m), 3.92-3.97 (1H, m), 4.85 (1H, d, J=5.9 Hz).

### Reference Example 20

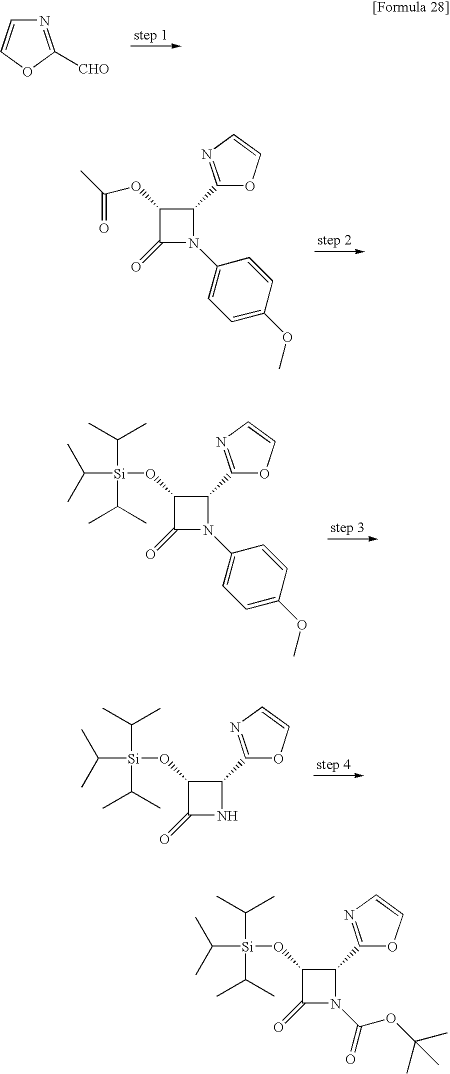

### Step 1: cis-3-Acetoxy-1-(4-methoxyphenyl)-4-(2-oxazolyl)-2-azetidinone

Oxazole-2-carboxyaldehyde (J. Org. Chem., 1991, 56, 449, 1.85 g) and p-anisidine (1.80 g) were dissolved in benzene (30 ml), and the solution was added with anhydrous sodium sulfate (5 g), and stirred at room temperature for 2 hours. The insoluble matters were removed by filtration, and then the reaction mixture was concentrated under reduced pressure to obtain an imine as yellow oil. A solution of the resulting imine and triethylamine (3.05 ml) in dichloromethane (20 ml) was cooled to −78° C. under a nitrogen atmosphere, then added dropwise with acetoxyacetyl chloride (1.57 ml), and warmed overnight to room temperature. The reaction mixture was concentrated, and then diluted with ethyl acetate. The reaction mixture was successively washed with saturated aqueous ammonium chloride, saturated aqueous sodium hydrogencarbonate and saturated brine, and then dried over anhydrous sodium sulfate, and the solvent was evaporated under reduced pressure. The resulting residue was purified by silica gel chromatography (developing solvent: ethyl acetate), and added with ether, and the deposited solid was collected by filtration, and dried to obtain the title compound (3.05 g) as colorless solid.

¹H-NMR (400 MHz, CDCl₃) δ: 1.96 (3H, s), 3.77 (3H, s), 5.51 (1H, d, J=4.9 Hz), 6.10 (1H, d, J=4.9 Hz), 6.82-6.86 (2H, m), 7.18 (1H, d, J=0.5 Hz), 7.25-7.28 (2H, m), 7.70 (1H, d, J=0.5 Hz).

ESI-MS; m/z: 303 (M+H)⁺.

### Step 2: cis-1-(4-Methoxyphenyl)-4-(2-oxazolyl)-3-[(triisopropylsilyl)oxy]-2-azetidinone

The compound obtained in Step 1 mentioned above (2.80 g) was dissolved in a mixture of tetrahydrofuran (28 ml) and methanol (28 ml), and the solution was added with potassium carbonate (50 mg), and stirred at room temperature for 60 minutes. The mixture was added with prepared Dowex 50 (strongly acidic resin), stirred for about 5 minutes, then filtered, concentrated, and azeotroped 2 times with toluene. The resulting residue was dissolved in N,N-dimethylformamide (30 ml), and the solution was added with imidazole (770 mg) and triisopropylsilyl chloride (2.40 ml) at 0° C., and the mixture was stirred at room temperature for 24 hours. The reaction mixture was added with water, and extracted with ethyl acetate, and the organic layer was washed with water and saturated brine, and dried over anhydrous sodium sulfate. Then, the solvent was evaporated under reduced pressure, and the resulting residue was purified by silica gel chromatography [developing solvent; ethyl acetate:n-hexane=1:5 (v/v)] to obtain the title compound (3.30 g) as colorless solid.

¹H-NMR (400 MHz, CDCl₃) δ: 0.97-1.01 (18H, m), 1.03-1.12 (3H, m), 3.76 (3H, s), 5.36 (2H, s), 6.80-6.84 (2H, m), 7.15 (1H, d, J=0.7 Hz), 7.23-7.25 (2H, m), 7.06 (1H, d, J=0.7 Hz).

### Step 3: cis-4-(2-Oxazolyl)-3-[(triisopropylsilyl)oxy]-2-azetidinone

The compound obtained in Step 2 mentioned above was used as a starting material to perform the same procedure as that of Reference Example 18, Step 2 and thereby to obtain the title compound as colorless solid.

¹H-NMR (400 MHz, CDCl₃) δ: 0.96-1.09 (21H, m), 4.98 (1H, d, J=4.9 Hz), 5.29 (1H, dd, J=4.9, 2.0 Hz), 6.34 (1H, br), 7.13 (1H, d, J=0.7 Hz), 7.69 (1H, d, J=0.7 Hz).

### Step 4: cis-1-(tert-Butoxycarbonyl)-4-(2-oxazolyl)-3-[(triisopropylsilyl)oxy]-2-azetidinone

The compound obtained in Step 3 mentioned above was used as a starting material to perform the same procedure as that of Reference Example 18, Step 4 and thereby to obtain the title compound as pale yellow solid.

¹H-NMR (CDCl₃) δ: 0.95-0.97 (18H, m), 1.00-1.08 (3H, m), 1.45 (9H, s), 5.24 (2H, AB type d, J=5.5 Hz), 7.15 (1H, s,), 7.68 (1H, s).

ESI-MS; m/z: 433 (M+Na)⁺.

### Reference Example 21

### (3R,4R)-4-(Difluoromethyl)-1-(isopropoxycarbonylamino)-3-[(triisopropylsilyl)oxy]-2-azetidinone

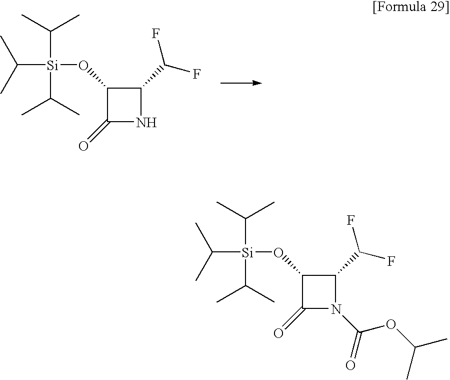

(3R,4R)-4-(Difluoromethyl)-3-[(triisopropylsilyl)oxy]-2-azetidinone (Bioorg. Med. Chem., 2000, 8, 1619, 400 mg) was dissolved in dichloromethane (8.0 ml), and the solution was cooled on ice. Then, the solution was added with N,N-diisopropylethylamine (0.28 ml), 4-dimethylaminopyridine (33.3 mg) and isopropyl chlorocarbonate (0.20 ml), and the mixture was stirred for 3 hours. The reaction mixture was added with ethyl acetate, and the organic layer was successively washed with 1 N aqueous hydrochloric acid, saturated aqueous sodium hydrogencarbonate, and saturated brine, and dried over anhydrous sodium sulfate. The solvent was evaporated under reduced pressure, and the resulting residue was purified by silica gel chromatography [developing solvent; ethyl acetate:n-hexane=10:90 (v/v)] to obtain the title compound (505 mg) as colorless oil.

¹H-NMR (400 MHz, CDCl₃) δ: 1.07-1.22 (21H, m), 1.32 (3H, d, J=6.3 Hz), 1.34 (3H, d, J=6.3 Hz), 4.28-4.35 (1H, m), 5.03-5.09 (1H, m), 5.16 (1H, dd, J=6.1, 1.0 Hz), 6.03 (1H, ddd, J=55.4, 54.0, 4.9 Hz).

ESI-MS; m/z: 402 (M+Na)⁺.

### Reference Example 22

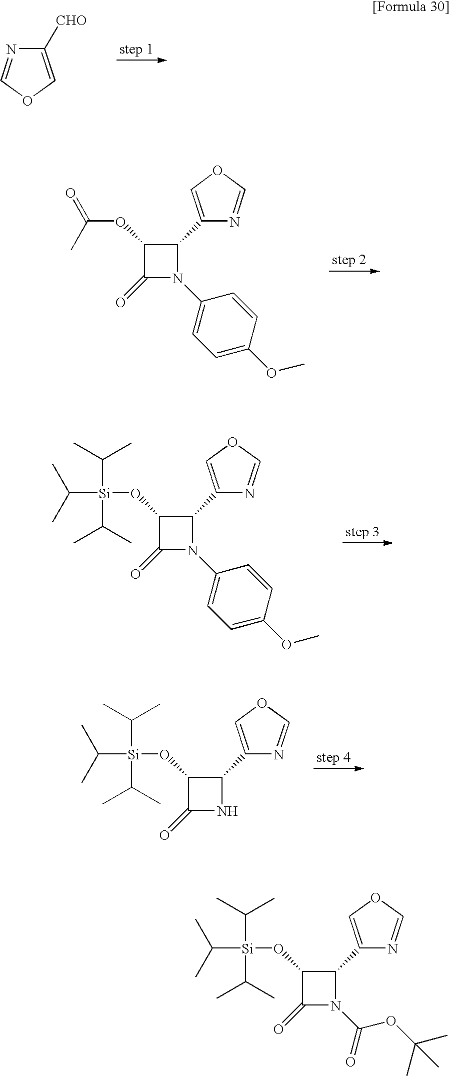

### Step 1: cis-3-Acetoxy-1-(4-methoxyphenyl)-4-(4-oxazolyl)-2-azetidinone

Oxazole-4-carboxyaldehyde (J. Org. Chem., 1991, 56, 449) was used as a starting material to perform the same procedure as that of Reference Example 20, Step 1 and thereby to obtain the title compound as pale yellow solid.

¹H-NMR (400 MHz, CDCl₃) δ: 1.99 (3H, s), 3.77 (3H, s), 5.41 (1H, d, J=4.9 Hz), 6.04 (1H, d, J=4.9 Hz), 6.82-6.86 (2H, m), 7.31-7.35 (2H, m), 7.68 (1H, t, J=0.9 Hz), 7.90 (1H, d, J=0.7 Hz).

### Step 2: cis-1-(4-Methoxyphenyl)-4-(4-oxazolyl)-3-[(triisopropylsilyl)oxy]-2-azetidinone

The compound obtained in Step 1 mentioned above was used as a starting material to perform the same procedure as that of Reference Example 20, Step 2 and thereby to obtain the title compound as pale yellow solid.

¹H-NMR (400 MHz, CDCl₃) δ: 0.98-1.24 (21H, m), 3.76 (3H, s), 5.24 (1H, d, J=4.9 Hz), 5.29 (1H, d, J=4.9 Hz), 6.80-6.84 (2H, m), 7.33-7.37 (2H, m), 7.67 (1H, s), 7.89 (1H, s).

ESI-MS; m/z: 439 (M+Na)⁺.

### Step 3: cis-4-(4-Oxazolyl)-3-[(triisopropylsilyl)oxy]-2-azetidinone

The compound obtained in Step 2 mentioned above was used as a starting material to perform the same procedure as that of Reference Example 18, Step 2 and thereby to obtain the title compound as pale brown solid.

¹H-NMR (400 MHz, CDCl₃) δ: 0.97-1.12 (21H, m), 4.87 (1H, d, J=4.2 Hz), 5.22 (1H, dd, J=4.9, 2.7 Hz), 6.06 (1H, brs), 7.74 (1H, s), 7.86 (1H, s).

ESI-MS; m/z: 333 (M+Na)⁺.

### Step 4: cis-1-(tert-Butoxycarbonyl)-4-(4-oxazolyl)-3-[(triisopropylsilyl)oxy]-2-azetidinone

The compound obtained in Step 3 mentioned above was used as a starting material to perform the same procedure as that of Reference Example 18, Step 4 and thereby to obtain the title compound as pale yellow solid.

¹H-NMR (CDCl₃) δ: 0.94-1.11 (21H, m), 1.47 (9H, s), 5.15 (1H, d, J=5.6 Hz), 5.19 (1H, d, J=5.6 Hz), 7.69 (1H, s), 7.87 (1H, s).

ESI-MS; m/z: 433 (M+Na)⁺.

### Reference Example 23

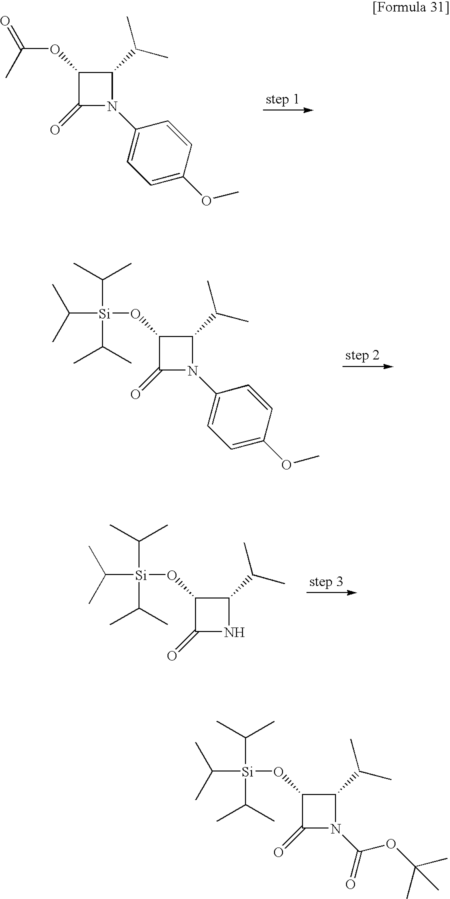

### Step 1: cis-4-Isopropyl-1-(4-methoxyphenyl)-3-[(triisopropylsilyl)oxy]-2-azetidinone

cis-3-Acetoxy-4-isopropyl-1-(4-methoxyphenyl)-2-azetidinone (Bioorg. Med. Chem., 2003, 11, 4315) was used as a starting material to perform the same procedure as that of Reference Example 20, Step 2 and thereby to obtain the title compound as colorless solid.

¹H-NMR (400 MHz, CDCl₃) δ: 1.02 (3H, d, J=6.8 Hz), 1.09 (3H, d, J=6.8 Hz), 1.11-1.14 (18H, m), 1.17-1.27 (3H, m), 2.28-2.35 (1H, m), 3.78 (3H, s), 4.07 (1H, t, J=5.3 Hz), 5.05 (1H, t, J=5.3 Hz), 6.83-6.87 (2H, m), 7.33-7.36 (2H, m).

### Step 2: cis-4-Isopropyl-3-[(triisopropylsilyl)oxy]-2-azetidinone

The compound obtained in Step 1 mentioned above was used as a starting material to perform the same procedure as that of Reference Example 18, Step 2 and thereby to obtain the title compound as pale brown solid.

¹H-NMR (400 MHz, CDCl₃) δ: 0.94-0.97 (6H, m), 1.08-1.11 (18H, m), 1.14-1.28 (3H, m), 1.90-1.99 (1H, m), 3.32 (1H, dd, J=8.6, 4.8 Hz), 4.98 (1H, dd, J=4.8, 2.7 Hz), 5.96 (1H, br).

### Step 3: cis-1-(tert-Butoxycarbonyl)-4-isopropyl-3-[(triisopropylsilyl)oxy]-2-azetidinone

The compound obtained in Step 2 mentioned above was used as a starting material to perform the same procedure as that of Reference Example 18, Step 4 and thereby to obtain the title compound as pale yellow solid.

¹H-NMR (CDCl₃) δ: 1.02 (6H, d, J=6.8 Hz), 1.07-1.11 (18H, m), 1.14-1.23 (3H, m), 1.52 (9H, s), 2.15-2.20 (1H, m), 3.89-3.92 (1H, m), 4.98 (1H, d, J=6.3 Hz).

### Reference Example 24

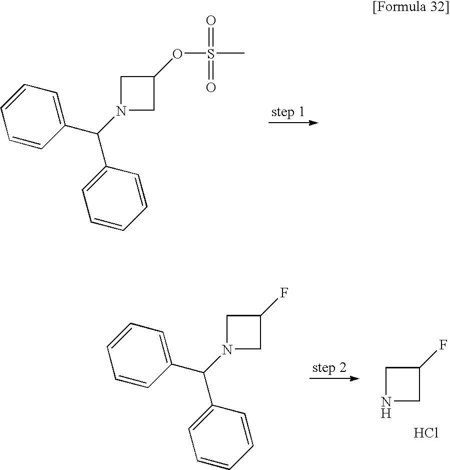

### Step 1: 1-Benzhydryl-3-fluoroazetidine

1-Benzhydryl-3-azetidinyl methanesulfonate (2.50 g) was dissolved in acetonitrile (50 ml), and the solution was added with tetra-n-butylammonium fluoride (1 mole solution in tetrahydrofuran, 7.9 ml), and refluxed for 6 hours by heating. The reaction mixture was concentrated, and then the resulting residue was diluted with ethyl acetate. The reaction mixture was successively washed with water (2 times) and saturated brine, and then dried over anhydrous sodium sulfate, and the solvent was evaporated under reduced pressure. The resulting residue was purified by silica gel chromatography [developing solvent; ethyl acetate:n-hexane=1:10 (v/v)] to obtain the title compound (1.40 g) as colorless solid.

¹H-NMR (400 MHz, CDCl₃) δ: 3.09-3.18 (2H, m), 3.50-3.58 (2H, m), 4.38 (1H, s), 5.06-5.24 (1H, m), 7.17-7.40 (10H, m).

### Step 2: 3-Fluoroazetidine hydrochloride

The compound obtained in Step 1 mentioned above was used as a starting material to perform the same procedure as that of Reference Example 1 and thereby to obtain the title compound as colorless solid.

¹H-NMR (400 MHz, d₆-DMSO) δ: 4.00-4.10 (2H, m), 4.22-4.32 (2H, m), 5.29-5.49 (1H, m), 9.66 (2H, br).

ESI-MS; m/z: 112 (M+H)⁺.

### Reference Example 25

### (S)-1-Azetidin-2-ylmethanol hydrochloride

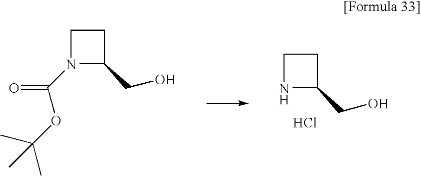

(S)-2-(Hydroxymethyl)-1-azetidinecarboxylic acid tert-butyl ester (4.20 g) was added with concentrated hydrochloric acid (14 ml), and the mixture was stirred at room temperature for 1 hour. The reaction mixture was concentrated under reduced pressure to obtain the title compound (2.68 g) as colorless oil.

¹H-NMR (400 MHz, d₆-DMSO) δ: 2.22-2.38 (2H, m), 3.36 (1H, brs), 3.70-3.74 (1H, m), 3.78-3.85 (1H, m), 4.34-4.36 (1H, m), 5.41-5.43 (1H, m), 8.90 (1H, brs), 9.33 (1H, brs).

### Reference Example 26

### (R)-1-Azetidin-2-ylmethanol hydrochloride

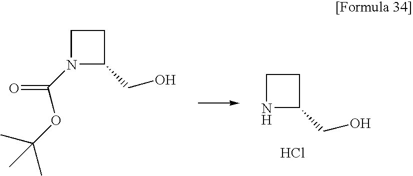

(R)-2-(Hydroxymethyl)-1-azetidinecarboxylic acid tert-butyl ester was used as a starting material to perform the same procedure as that of Reference Example 25 and thereby to obtain the title compound as colorless oil.

¹H-NMR (400 MHz, CDCl₃) δ: 2.45-2.65 (2H, m), 3.83-4.05 (4H, m), 4.62 (1H, brs), 5.62 (1H, brs), 9.06 (1H, brs), 9.57 (1H, brs).

### Reference Example 27

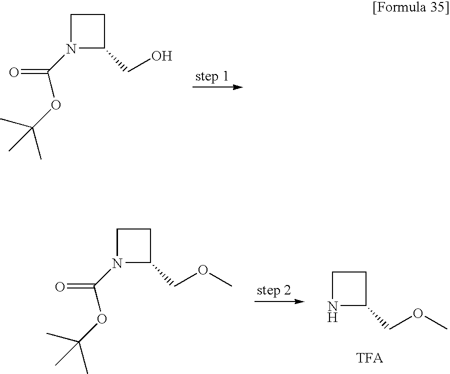

### Step 1: (R)-2-(Methoxymethyl)-1-azetidinecarboxylic acid tert-butyl ester

(R)-2-(Hydroxymethyl)-1-azetidinecarboxylic acid tert-butyl ester was used as a starting material to perform the same procedure as that of Reference Example 11, Step 1 and thereby to obtain the title compound as colorless oil.

¹H-NMR (400 MHz, CDCl₃) δ: 1.44 (9H, s), 2.11-2.27 (2H, m), 3.41 (3H, s), 3.54 (1H, dd, J=10.1, 3.1 Hz), 3.63-3.67 (1H, m), 3.82 (2H, dd, J=8.2, 6.7 Hz), 4.27-4.33 (1H, m).

### Step 2: (R)-2-(Methoxymethyl)azetidine trifluoroacetate

The compound obtained in Step 1 mentioned above was used as a starting material to perform the same procedure as that of Reference Example 11, Step 2 and thereby to obtain the title compound as colorless oil.

¹H-NMR (400 MHz, d₆-DMSO) δ: 2.20-2.29 (1H, m), 2.33-2.42 (1H, m), 3.32 (3H, s), 3.53 (1H, dd, J=11.2, 3.4 Hz), 3.64 (1H, dd, J=11.2, 7.1 Hz), 3.69-3.79 (1H, m), 3.83-3.93 (1H, m), 4.50 (1H, brs), 8.81 (2H, brs).

ESI-MS; m/z: 102 (M+H)⁺.

### Reference Example 28

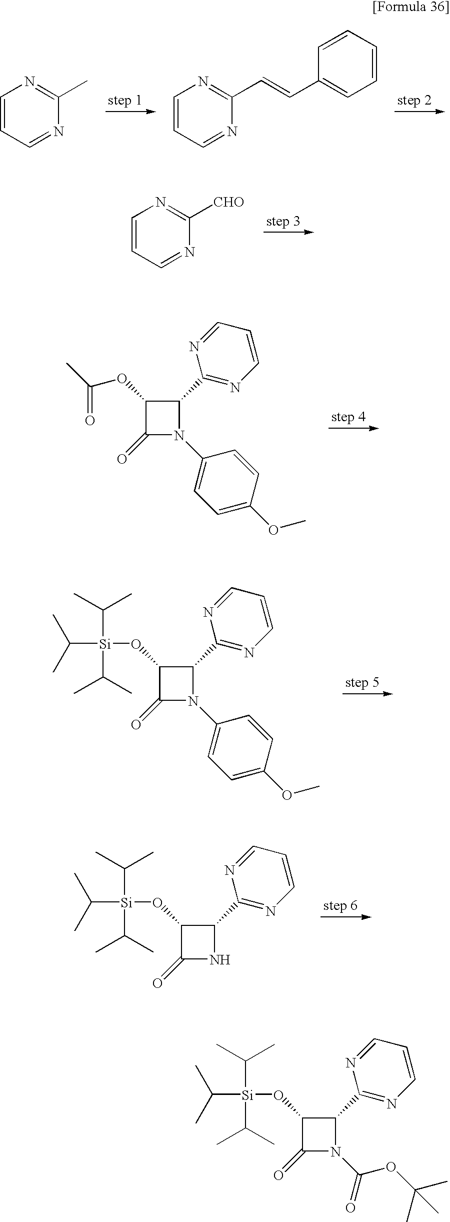

### Step 1: 2-[(E)-Styryl]pyrimidine

A mixture of 2-methylpyrimidine (5.00 g) and benzaldehyde (8.10 ml) was added with zinc chloride (1 mole solution in ether, 7.97 ml) at room temperature, and refluxed at 160° C. by heating for 23 hours. The reaction mixture was cooled to room temperature, and then purified by silica gel column chromatography [developing solvent; ethyl acetate:n-hexane=1:9 to 3:7 (v/v)] to obtain the title compound (6.85 g) as brown solid.

¹H-NMR (400 MHz, CDCl₃) δ: 7.11 (1H, t, J=4.9 Hz), 7.25 (1H, d, J=16.1 Hz), 7.32-7.42 (3H, m), 7.62-7.64 (2H, m), 8.00 (1H, d, J=16.1 Hz), 8.73 (2H, d, J=4.6 Hz).

ESI-MS; m/z: 183 (M+Na)⁺.

### Step 2: Pyrimidine-2-carboxyaldehyde

The compound obtained in Step 1 mentioned above (6.62 g) was dissolved in 1,4-dioxane (90 ml) and water (30 ml), and the solution was added with osmium tetroxide (catalytic amount) and sodium metaperiodate (15.6 g) at room temperature. The reaction mixture was stirred for 2 days, and filtered through Celite, and then the solvent was evaporated under reduced pressure. The resulting residue was purified by silica gel column chromatography [developing solvent; ethyl acetate:n-hexane=2:1 to 100:0 (v/v)] to obtain the title compound (3.27 g) as brown solid.

¹H-NMR (400 MHz, CDCl₃) δ: 7.52 (1H, t, J=4.9 Hz), 9.01 (2H, d, J=4.9 Hz), 10.14 (1H, s).

ESI-MS; m/z: 109 (M+Na)⁺.

### Step 3: cis-3-Acetoxy-1-(4-methoxyphenyl)-4-(2-pyrimidinyl)-2-azetidinone

The compound obtained in Step 2 mentioned above was used as a starting material to perform the same procedure as that of Reference Example 20, Step 1 and thereby to obtain the title compound as brown solid.

¹H-NMR (400 MHz, CDCl₃) δ: 1.80 (3H, s), 3.76 (3H, s), 5.56 (1H, d, J=5.1 Hz), 6.20 (1H, d, J=5.1 Hz), 6.82 (2H, dt, J=9.8, 2.9 Hz), 7.26-7.30 (3H, m), 8.77 (2H, d, J=4.9 Hz).

ESI-MS; m/z: 314 (M+Na)⁺.

### Step 4: cis-1-(4-Methoxyphenyl)-4-(2-pyrimidinyl)-3-[(triisopropylsilyl)oxy]-2-azetidinone

The compound obtained in Step 3 mentioned above was used as a starting material to perform the same procedure as that of Reference Example 20, Step 2 and thereby to obtain the title compound as colorless solid.

¹H-NMR (400 MHz, CDCl₃) δ: 0.98-1.24 (21H, m), 3.76 (3H, s), 5.24 (1H, d, J=4.9 Hz), 5.29 (1H, d, J=4.9 Hz), 6.80-6.84 (2H, m), 7.33-7.37 (2H, m), 7.67 (1H, s), 7.89 (1H, s).

ESI-MS; m/z: 428 (M+Na)⁺.

### Step 5: cis-4-(2-Pyrimidinyl)-3-[(triisopropylsilyl)oxy]-2-azetidinone

The compound obtained in Step 4 mentioned above was used as a starting material to perform the same procedure as that of Reference Example 18, Step 2 and thereby to obtain the title compound as pale yellow oil.

¹H-NMR (400 MHz, CDCl₃) δ: 0.90-1.06 (21H, m), 5.05 (1H, d, J=4.9 Hz), 5.36 (1H, dd, J=4.9, 1.5 Hz), 6.28 (1H, brs), 7.22 (1H, t, J=4.9 Hz), 8.74 (2H, d, J=4.9 Hz).

ESI-MS; m/z: 322 (M+Na)⁺.

### Step 6: cis-1-(tert-Butoxycarbonyl)-4-(2-pyrimidinyl)-3-[(triisopropylsilyl)oxy]-2-azetidinone

The compound obtained in Step 5 mentioned above was used as a starting material to perform the same procedure as that of Reference Example 18, Step 4 and thereby to obtain the title compound as colorless solid.

¹H-NMR (CDCl₃) δ: 0.88-1.06 (21H, m), 1.45 (9H, s), 5.28 (1H, d, J=5.9 Hz), 5.31 (1H, d, J=5.9 Hz), 7.22 (1H, t, J=4.9 Hz), 8.73 (2H, d, J=4.9 Hz).

### Reference Example 29

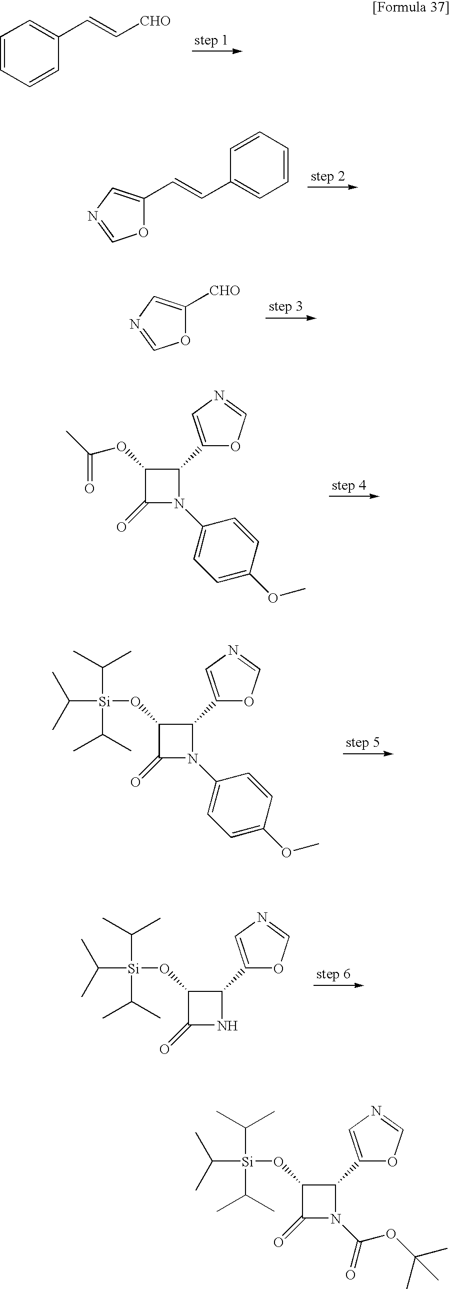

### Step 1: 5-[(E)-Styryl]oxazole

A suspension of trans-cinnamaldehyde (4.0 g) and (p-toluenesulfonyl) methylisocyanide (5.9 g) in methanol (100 ml) was added with potassium carbonate (5.0 g), and the mixture was stirred at 80° C. for 2 hours. The reaction mixture was concentrated under reduced pressure, and the resulting residue was added with water and ethyl acetate. Two of the layers were separated, and the aqueous layer was extracted with ethyl acetate. The organic layers were combined, washed with saturated brine, and then dried over anhydrous sodium sulfate, and the solvent was evaporated under reduced pressure. The resulting residue was purified by silica gel column chromatography [developing solvent; ethyl acetate:n-hexane=1:3 (v/v)] to obtain the title compound (4.4 g) as orange solid.

¹H-NMR (400 MHz, CDCl₃) δ: 6.92 (1H, d, J=16.4 Hz), 7.08 (1H, s), 7.11 (1H, d, J=16.1 Hz), 7.29 (1H, t, J=7.3 Hz), 7.37 (2H, t, J=7.6 Hz), 7.48 (2H, d, J=7.8 Hz), 7.84 (1H, s).

ESI-MS; m/z: 172 (M+H)⁺.

### Step 2: Oxazole-5-carboxyaldehyde

The compound obtained in Step 1 mentioned above was used as a starting material to perform the same procedure as that of Reference Example 28, Step 2 and thereby to obtain the title compound as yellow oil.

¹H-NMR (400 MHz, CDCl₃) δ: 7.89 (1H, s), 8.12 (1H, s), 9.87 (1H, d, J=0.5 Hz).

### Step 3: cis-3-Acetoxy-1-(4-methoxyphenyl)-4-(5-oxazolyl)-2-azetidine

The compound obtained in Step 2 mentioned above was used as a starting material to perform the same procedure as that of Reference Example 20, Step 1 and thereby to obtain the title compound as brown solid.

¹H-NMR (400 MHz, CDCl₃) δ: 1.98 (3H, s), 3.77 (3H, s), 5.48 (1H, d, J=4.6 Hz), 6.02 (1H, d, J=4.6 Hz), 6.84 (2H, d, J=9.0 Hz), 7.18 (1H, s), 7.28 (2H, d, J=8.8 Hz), 7.91 (1H, s).

ESI-MS; m/z: 303 (M+H)⁺.

### Step 4: cis-1-(4-Methoxyphenyl)-4-(5-oxazolyl)-3-[(triisopropylsilyl)oxy]-2-azetidinone

The compound obtained in Step 3 mentioned above was used as a starting material to perform the same procedure as that of Reference Example 20, Step 2 and thereby to obtain the title compound as pale yellow solid.

¹H-NMR (400 MHz, CDCl₃) δ: 0.96-1.16 (21H, m), 3.76 (3H, s), 5.30 (2H, s), 6.82 (2H, d, J=9.7 Hz), 7.16 (1H, s), 7.29 (2H, d, J=9.0 Hz), 7.88 (1H, s).

FAB-MS; m/z: 417 (M+H)⁺.

### Step 5: cis-4-(5-Oxazolyl)-3-[(triisopropylsilyl)oxy]-2-azetidinone

The compound obtained in Step 4 mentioned above was used as a starting material to perform the same procedure as that of Reference Example 18, Step 2 and thereby to obtain the title compound as brown solid.

¹H-NMR (400 MHz, CDCl₃) δ: 0.95-1.12 (21H, m), 4.93 (1H, d, J=4.6 Hz), 5.24 (1H, dd, J=4.6, 2.4 Hz), 6.04 (1H, brs), 7.13 (1H, s), 7.88 (1H, s).

ESI-MS; m/z: 311 (M+H)⁺.

### Step 6: cis-1-(tert-Butoxycarbonyl)-4-(5-oxazolyl)-3-[(triisopropylsilyl)oxy]-2-azetidinone

The compound obtained in Step 5 mentioned above was used as a starting material to perform the same procedure as that of Reference Example 18, Step 4 and thereby to obtain the title compound as colorless solid.

¹H-NMR (CDCl₃) δ: 0.93-1.11 (211H, m), 1.46 (9H, s), 5.21 (2H, s), 7.15 (1H, s), 7.89 (1H, s).

ESI-MS; m/z: 411 (M+H)⁺.

### Reference Example 30

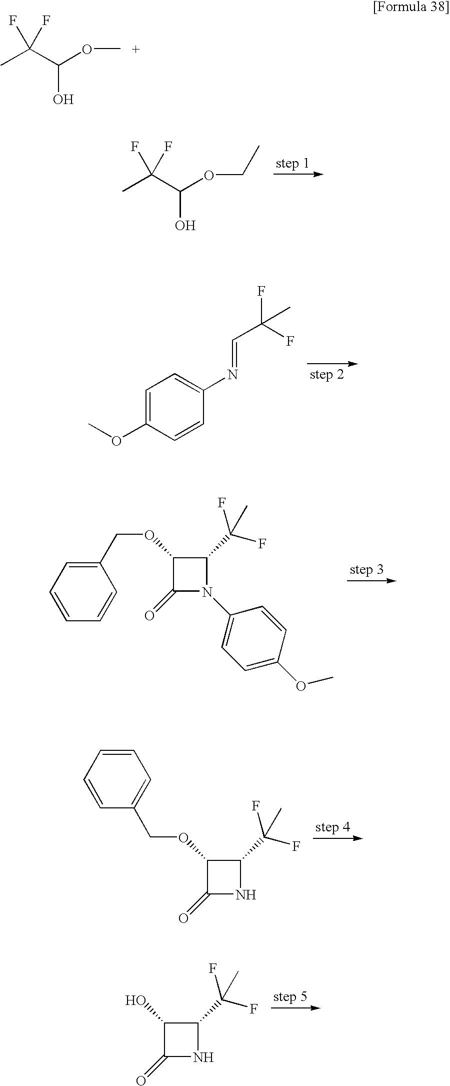

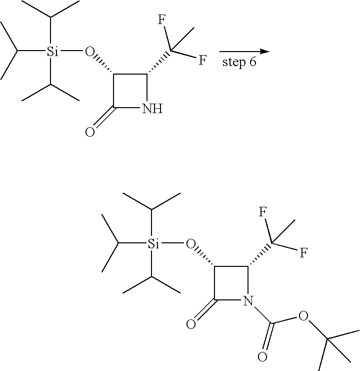

### Step 1: N-(2,2-Difluoropropylidene)-4-methoxyaniline

A mixture of 1-ethoxy-2,2-difluoro-1-propanol and 1-methoxy-2,2-difluoro-1-propanol (J. Org. Chem., 1995, 60, 5174, 722 mg) was dissolved in toluene, and the solution was added with p-anisidine (0.52 g), and refluxed by heating for 3 hours. The reaction mixture was cooled to room temperature, then added with saturated aqueous sodium hydrogencarbonate, and extracted with toluene. The organic layer was washed with saturated brine, and dried over anhydrous magnesium sulfate, and then the solvent was evaporated under reduced pressure to obtain the title compound (989 mg) as yellow oil.

¹H-NMR (400 MHz, CDCl₃) δ: 1.89 (3H, t, J=18.8 Hz), 3.83 (3H, s), 6.89-6.94 (2H, m), 7.15-7.28 (2H, m), 7.87 (1H, t, J=3.4 Hz).

### Step 2: cis-1-(4-Methoxyphenyl)-3-(benzyloxy)-4-(1,1-difluoroethyl)-2-azetidinone

The compound obtained in Step 1 mentioned above (837 mg) was dissolved in dichloromethane (4.94 ml), and the solution was added with triethylamine (2.05 ml) at 0° C., then added dropwise with benzyloxyacetyl chloride (1.55 g), and stirred at 45° C. for 84 hours. Then, the reaction mixture was added with water, and extracted with ethyl acetate. The organic layer was washed with saturated brine, and dried over anhydrous magnesium sulfate, and the solvent was evaporated under reduced pressure. The resulting residue was added with ether, and the deposited solid was collected by filtration, and dried to obtain the title compound (931 mg) as pale brown solid.

¹H-NMR (400 MHz, CDCl₃) δ: 1.77 (3H, t, J=19.5 Hz), 3.79 (3H, s), 4.48-4.52 (1H, m), 4.77-4.84 (1H, m), 4.91 (1H, d, J=5.1 Hz), 6.85-6.90 (2H, m), 7.30-7.41 (5H, m), 7.43-7.48 (2H, m).

### Step 3: cis-3-(Benzyloxy)-4-(1,1-difluoroethyl)-2-azetidinone

The compound obtained in Step 2 mentioned above was used as a starting material to perform the same procedure as that of Reference Example 18, Step 2 and thereby to obtain the title compound as pale yellow solid.

¹H-NMR (400 MHz, CDCl₃) δ: 1.76 (3H, t, J=19.3 Hz), 3.95-4.02 (1H, m), 4.72-4.84 (2H, m), 6.05 (1H, brs), 7.30-7.38 (5H, m).

### Step 4: cis-4-(1,1-Difluoroethyl)-3-hydroxy-2-azetidinone

The compound obtained in Step 3 mentioned above was used as a starting material to perform the same procedure as that of Reference Example 19, Step 1 and thereby to obtain the title compound as pale yellow amorphous solid.

¹H-NMR (400 MHz, CDCl₃) δ: 1.77 (3H, t, J=19.1 Hz), 2.87 (1H, brs), 3.89-3.97 (1H, m), 5.01-5.05 (1H, m), 6.07 (1H, brs).

### Step 5: cis-4-(1,1-Difluoroethyl)-3-[(triisopropylsilyl)oxy]-2-azetidinone

The compound obtained in Step 4 mentioned above was used as a starting material, and reacted with triisopropylsilyl chloride instead of triethylsilyl chloride to perform the same procedure as that of Reference Example 19, Step 2 and thereby to obtain the title compound as pale yellow oil.

¹H-NMR (400 MHz, CDCl₃) δ: 0.97-1.28 (21H, m), 1.77 (3H, t, J=19.3 Hz), 3.89-3.99 (1H, m), 5.09-5.14 (1H, m), 5.96 (1H, brs).

### Step 6: cis-1-(tert-Butoxycarbonyl)-4-(1,1-difluoroethyl)-3-[(triisopropylsilyl)oxy]-2-azetidinone

The compound obtained in Step 5 mentioned above was used as a starting material to perform the same procedure as that of Reference Example 18, Step 4 and thereby to obtain the title compound as colorless solid.

¹H-NMR (CDCl₃) δ: 0.97-1.28 (21H, m), 1.52 (9H, s), 1.76 (3H, t, J=19.0 Hz), 4.29-4.36 (1H, m), 5.13 (1H, d, J=6.3 Hz).

### Reference Example 31

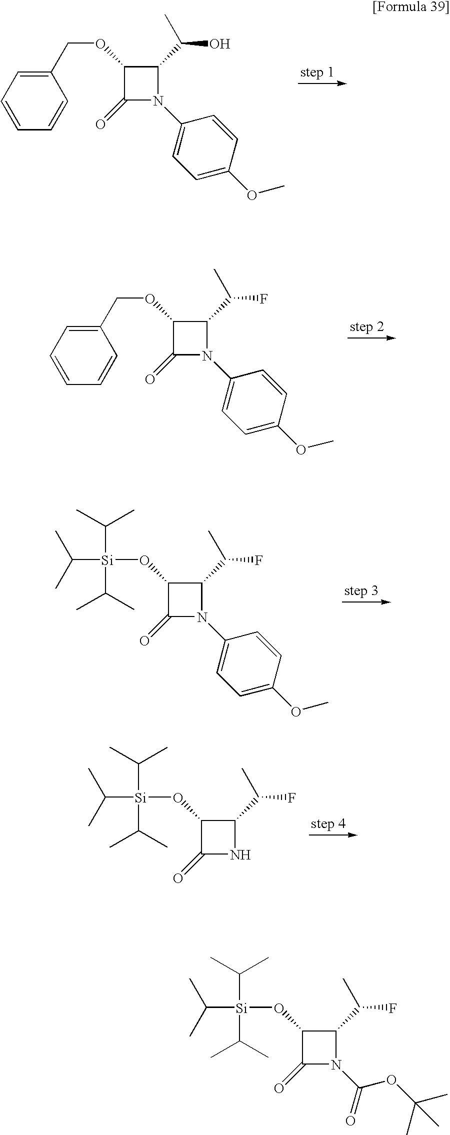

### Step 1: (3R,4R)-3-(Benzyloxy)-4-[(1S)-1-fluoroethyl]-1-(4-methoxyphenyl)-2-azetidinone

(3R,4S)-3-(Benzyloxy)-4-[(1R)-hydroxyethyl]-1-(4-methoxyphenyl)-2-azetidinone (J. Org. Chem., 1994, 59, 3123) was used as a starting material to perform the same procedure as that of Reference Example 18, Step 1 and thereby to obtain the title compound yellow solid.

¹H-NMR (400 MHz, CDCl₃) δ: 1.41 (3H, dd, J=25.1, 6.3 Hz), 3.79 (3H, s), 4.47-4.54 (1H, m), 4.73 (1H, d, J=11.7 Hz), 4.85 (1H, d, J=5.6 Hz), 4.89-5.11 (2H, m), 6.88 (2H, d, J=9.0 Hz), 7.28-7.52 (7H, m).

### Step 2: (3R,4R)-4-[(1S)-1-Fluoroethyl]-1-(4-methoxyphenyl)-3-[(triisopropylsilyl)oxy]-2-azetidinone

The compound obtained in Step 1 mentioned above was used as a starting material to perform the same procedure as that of Reference Example 18, Step 3 and thereby to obtain the title compound as colorless oil.

¹H-NMR (400 MHz, CDCl₃) δ: 1.08-1.27 (21H, m), 1.41 (3H, dd, J=25.1, 6.3 Hz), 3.79 (3H, s), 4.52 (1H, dd, J=16.1, 5.6 Hz), 4.97-5.16 (1H, m), 5.13 (1H, d, J=5.6 Hz), 6.87 (2H, d, J=9.0 Hz), 7.51 (2H, d, J=9.0 Hz).

ESI-MS; m/z: 396 (M+H)⁺.

### Step 3: (3R,4R)-4-[(1S)-1-Fluoroethyl]-3-[(triisopropylsilyl)oxy]-2-azetidinone

The compound obtained in Step 2 mentioned above was used as a starting material to perform the same procedure as that of Reference Example 18, Step 2 and thereby to obtain the title compound as orange oil.

¹H-NMR (400 MHz, CDCl₃) δ: 1.05-1.22 (21H, m), 1.43 (3H, dd, J=24.9, 6.3 Hz), 3.94 (1H, dt, J=12.9, 4.9 Hz), 4.81-5.01 (1H, m), 5.07 (1H, dd, J=5.1, 2.7 Hz), 5.90 (1H, brs).

ESI-MS; m/z: 290 (M+H)⁺.

### Step 4: (3R,4R)-1-(tert-Butoxycarbonyl)-4-[(1S)-1-fluoroethyl]-3-[(triisopropylsilyl)oxy]-2-azetidinone

The compound obtained in Step 3 mentioned above was used as a starting material to perform the same procedure as that of Reference Example 18, Step 4 and thereby to obtain the title compound as colorless oil.

¹H-NMR (400 MHz, CDCl₃) δ: 1.04-1.22 (21H, m), 1.46 (3H, dd, J=24.4, 6.6 Hz), 1.52 (9H, s), 4.24-4.32 (1H, m), 4.90-5.09 (1H, m), 5.08 (1H, d, J=6.1 Hz).

ESI-MS; m/z: 412 (M+Na)⁺.

### Reference Example 32

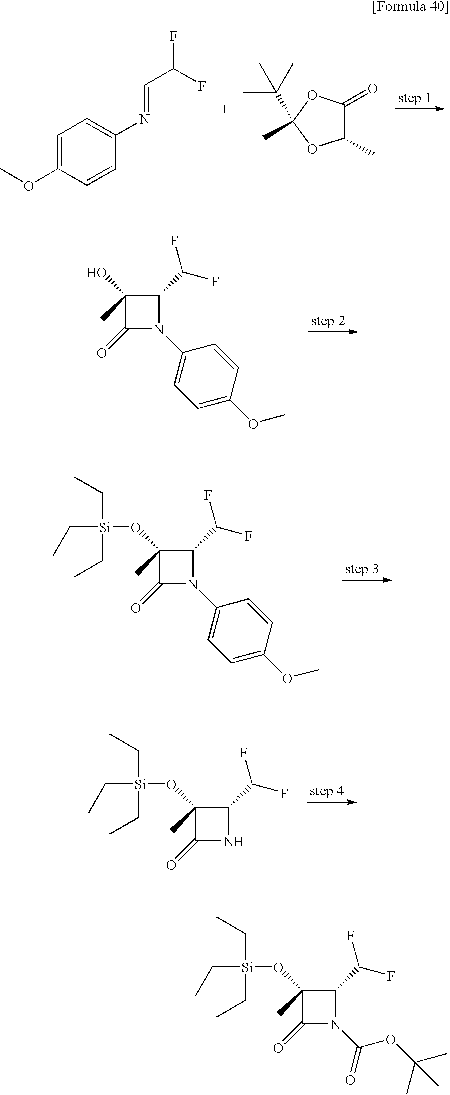

### Step 1: (3R,4R)-4-Difluoromethyl-3-hydroxy-1-(4-methoxyphenyl)-3-methyl-2-azetidinone

A solution of (2S,5S)-2-(tert-butyl)-2,5-dimethyl-1,3-dioxolan-4-one (J. Am. Chem. Soc., 1995, 117, 6394, 1.55 g) in tetrahydrofuran (20 ml) was added with lithium bis(trimethylsilyl)amide (1 mole solution in tetrahydrofuran, 10.8 ml) at −78° C. under an argon flow, and the mixture was stirred at the same temperature for 30 minutes. The reaction mixture was added with a solution of hexamethylphosphoramide (6.0 ml) and N-(2,2-difluoroethylidene)-4-methoxyaniline (J. Org. Chem., 1997, 62, 8826, 3.3 g) in tetrahydrofuran (3.2 ml), and stirred at the same temperature for 4 hours. The reaction mixture was added with saturated aqueous ammonium chloride, and then extracted with ethyl acetate. The extract was successively washed with 1 N aqueous hydrochloric acid and saturated brine, and then dried over anhydrous sodium sulfate, and the solvent was evaporated under reduced pressure. The resulting residue was purified by silica gel column chromatography [developing solvent; ethyl acetate:n-hexane=1:4 (v/v)] to obtain the title compound (0.86 g) as colorless solid.

¹H-NMR (400 MHz, CDCl₃) δ: 1.69 (3H, s), 3.02 (1H, brs), 3.80 (3H, s), 4.08-4.16 (1H, m), 6.11 (1H, ddd, J=57.2, 52.1, 3.6 Hz), 6.89 (2H, d, J=9.0 Hz), 7.44 (2H, d, J=8.8 Hz).

FAB-MS; m/z: 258 (M+H)⁺.

### Step 2: (3R,4R)-4-Difluoromethyl-1-(4-methoxyphenyl)-3-methyl-3-[(triethylsilyl)oxy]-2-azetidinone

The compound obtained in Step 1 mentioned above was used as a starting material to perform the same procedure as that of Reference Example 19, Step 2 and thereby to obtain the title compound as colorless oil.

¹H-NMR (400 MHz, CDCl₃) δ: 0.68-0.80 (6H, m), 0.99 (9H, t, J=7.8 Hz), 1.57 (3H, s), 3.79 (3H, s), 3.96-4.03 (1H, m), 5.97 (1H, ddd, J=57.7, 52.5, 3.8 Hz), 6.85-6.90 (2H, m), 7.44 (2H, d, J=9.3 Hz).

### Step 3: (3R,4R)-4-Difluoromethyl-3-methyl-3-[(triethylsilyl)oxy]-2-azetidinone

The compound obtained in Step 2 mentioned above was used as a starting material to perform the same procedure as that of Reference Example 18, Step 2 and thereby to obtain the title compound as orange oil.

¹H-NMR (400 MHz, CDCl₃) δ: 0.66-0.77 (6H, m), 0.97 (9H, t, J=8.1 Hz), 1.59 (3H, s), 3.52-3.59 (1H, m), 5.81 (1H, ddd, J=57.6, 50.8, 3.4 Hz), 5.90 (1H, brs).

### Step 4: (3R,4R)-1-(tert-Butoxycarbonyl)-4-difluoromethyl-3-methyl-3-[(triethylsilyl)oxy]-2-azetidinone

The compound obtained in Step 3 mentioned above was used as a starting material to perform the same procedure as that of Reference Example 18, Step 4 and thereby to obtain the title compound as orange oil.

¹H-NMR (400 MHz, CDCl₃) δ: 0.59-0.77 (6H, m), 0.96 (9H, t, J=7.8 Hz), 1.52 (9H, s), 1.57 (3H, s), 3.86-3.94 (1H, m), 5.91 (1H, ddd, J=57.1, 52.7, 3.2 Hz).

### Reference Example 33

### (3R,4R)-1-Benzoyl-4-difluoromethyl-3-methyl-3-[(triethylsilyl)oxy]-2-azetidinone

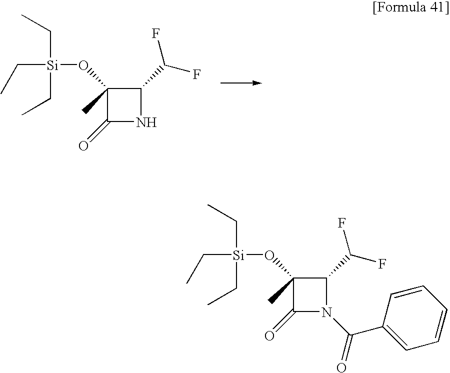

A solution of the compound obtained in Reference Example 32, Step 3 (0.17 g) in dichloromethane (5 ml) was added with triethylamine (134 μl), benzoyl chloride (112 μl) and 4-dimethylaminopyridine (16 mg), and the mixture was stirred at room temperature for 1 hour. The reaction mixture was added with saturated aqueous ammonium chloride, and then two of the layers were separated. The aqueous layer was extracted with chloroform, and the organic layers were combined, washed with saturated aqueous sodium hydrogencarbonate and saturated brine, and then dried over anhydrous sodium sulfate. The solvent was evaporated under reduced pressure, and the resulting residue was purified by silica gel column chromatography [developing solvent; ethyl acetate:n-hexane=1:8 (v/v)] to obtain the title compound (0.2 g) as yellow oil.

¹H-NMR (400 MHz, CDCl₃) δ: 0.67-0.79 (6H, m), 1.00 (9H, t, J=7.8 Hz), 1.63 (3H, s), 4.31-4.39 (1H, m), 6.06 (1H, dt, J=54.7, 4.2 Hz), 7.49 (2H, t, J=8.1 Hz), 7.62 (1H, t, J=7.3 Hz), 7.96 (2H, d, J=8.3 Hz).

### Reference Example 34

### cis-4-(1,1-Difluoroethyl)-1-(isopropoxycarbonyl)-3-[(triisopropylsilyl)oxy]-2-azetidinone

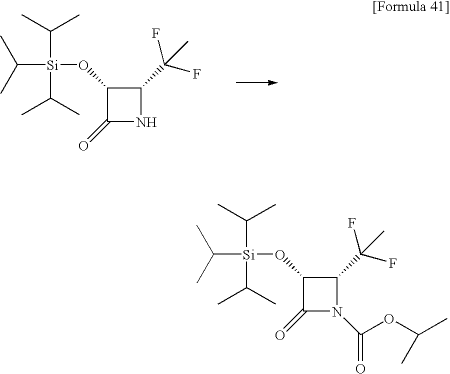

The compound obtained in Reference Example 30, Step 5 was used as a starting material to perform the same procedure as that of Reference Example 21 and thereby to obtain the title compound as colorless oil.

¹H-NMR (400 MHz, CDCl₃) δ: 1.04-1.21 (21H, m), 1.25-1.38 (6H, m), 1.76 (3H, dt, J=19.0, 6.3 Hz), 4.33-4.40 (1H, m), 5.01-5.10 (1H, m), 5.15 (1H, d, J=6.1 Hz).

### Reference Example 35

### (3R,4S)-3-[(tert-Butyldimethylsilyl)oxy]-1-(isopropoxycarbonyl)-4-phenyl-2-azetidinone

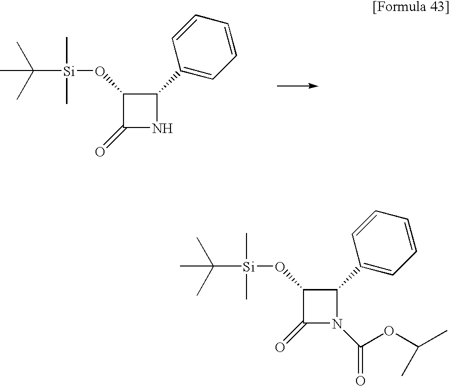

(3R,4S)-3-[(tert-Butyldimethylsilyl)oxy]-4-phenyl-2-azetidinone (Tetrahedron, 1992, 48, 6985) was used as a starting material to perform the same procedure as that of Reference Example 21 and thereby to obtain the title compound as colorless solid.

¹H-NMR (400 MHz, CDCl₃) δ: −0.16 (3H, s), 0.05 (3H, s), 0.64 (9H, s), 1.16 (3H, d, J=6.1 Hz), 1.27 (3H, d, J=6.6 Hz), 4.93-5.01 (1H, m), 5.06 (1H, d, J=5.6 Hz), 5.09 (1H, d, J=5.6 Hz), 7.25-7.37 (5H, m).

### Reference Example 36

### (3R,4R)-1-(Isopropoxycarbonyl)-4-(trifluoromethyl)-3-[(triisopropylsilyl)oxy]-2-azetidinone

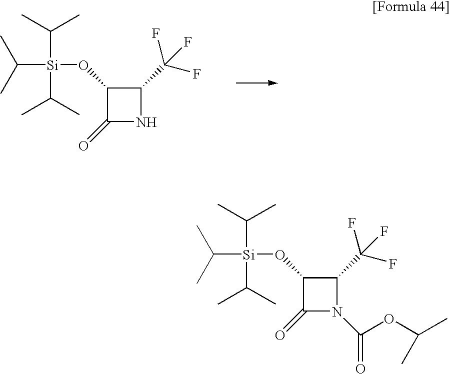

(3R,4R)-4-(Trifluoromethyl)-3-[(triisopropylsilyl)oxy]-2-azetidinone (J. Fluorine. Chem., 2004, 125, 487) was used as a starting material to perform the same procedure as that of Reference Example 21 and thereby to obtain the title compound as pale yellow oil.

¹H-NMR (400 MHz, CDCl₃) δ: 1.03-1.22 (21H, m), 1.32 (3H, d, J=6.2 Hz), 1.34 (3H, d, J=6.2 Hz), 4.49-4.54 (1H, m), 5.03-5.09 (1H, m), 5.23 (1H, d, J=6.2 Hz).

ESI-MS; m/z: 420 (M+Na)⁺.

### Reference Example 37

### 2-[(2S)-Azetidinyl]-2-propanol hydrochloride

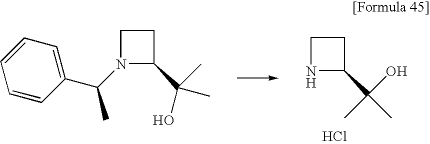

2-{(2S)-1-[(1S)-Phenylethyl]azetidinyl}-2-propanol (Tetrahedron, 1998, 54, 4991) was used as a starting material to perform the same procedure as that of Reference Example 1 and thereby to obtain the title compound as off-white solid.

ESI-MS; m/z: 116 (M+Na)⁺.

### Reference Example 38

### (3R,4S)-4-(3-Fluoro-2-pyridyl)-1-(isopropoxycarbonyl)-3-[(triisopropylsilyl)oxy]-2-azetidinone

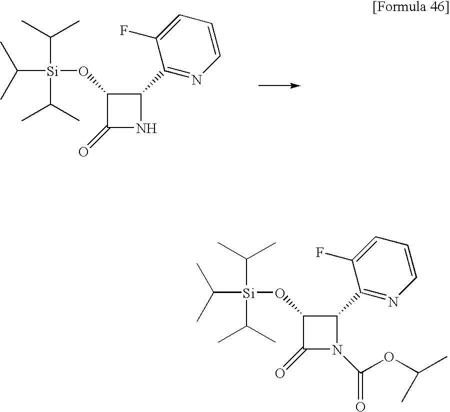

(3R,4S)-4-(3-Fluoro-2-pyridyl)-3-[(triisopropylsilyl)oxy]-2-azetidinone (Bioorg. Med. Chem. Lett., 2004, 14, 3209) was used as a starting material to perform the same procedure as that of Reference Example 21 and thereby to obtain the title compound as pale yellow oil.

¹H-NMR (400 MHz, CDCl₃) δ: 0.87-1.04 (21H, m), 1.20 (3H, d, J=6.1 Hz), 1.30 (3H, d, J=6.1 Hz), 4.97-5.05 (1H, m), 5.30 (1H, d, J=5.9 Hz), 5.50 (1H, d, J=5.6 Hz), 7.22-7.28 (1H, m), 7.38 (1H, t, J=9.3 Hz), 8.42 (1H, d, J=4.6 Hz).

### Reference Example 39

### (3R,4S)-3-[(tert-Butyldimethylsilyl)oxy]-1-(isopropoxycarbonyl)-3-methyl-4-(2-pyridyl)-2-azetidinone

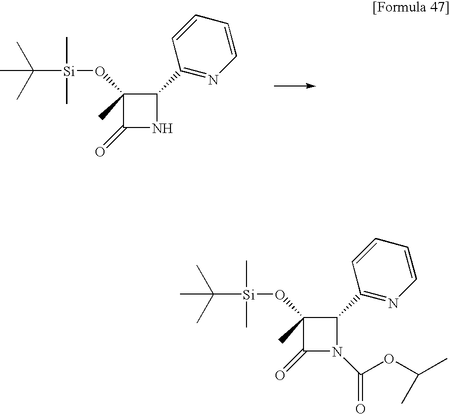

(3R,4S)-3-[(tert-Butyldimethylsilyl)oxy]-3-methyl-4-(2-pyridyl)-2-azetidinone (Bioorg. Med. Chem. Lett., 2003, 13, 185) was used as a starting material to perform the same procedure as that of Reference Example 21 and thereby to obtain the title compound as colorless oil.

¹H-NMR (400 MHz, CDCl₃) δ: 0.02 (3H, s), 0.08 (3H, s), 0.49 (9H, s), 1.20 (3H, d, J=6.3 Hz), 1.30 (3H, d, J=6.1 Hz), 1.72 (3H, s), 4.95 (1H, s), 5.00-5.07 (1H, m), 7.19-7.22 (2H, m), 7.67 (1H, dt, J=7.7, 1.7 Hz), 8.57-8.60 (1H, m).

ESI-MS; m/z: 379 (M+H)⁺.

### Reference Example 40

### (3R,4R)-4-Difluoromethyl-1-(isopropoxycarbonyl)-3-methyl-3-[(triethylsilyl)oxy]-2-azetidinone

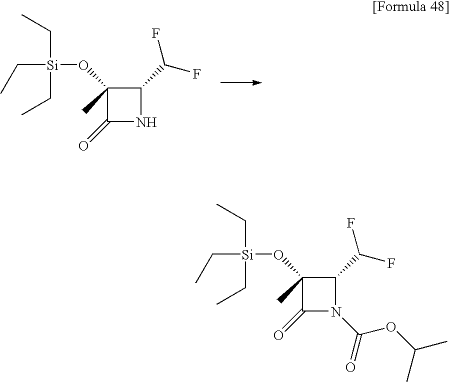

The compound obtained in Reference Example 32, Step 3 was used as a starting material to perform the same procedure as that of Reference Example 21 and thereby to obtain the title compound as colorless oil.

¹H-NMR (400 MHz, CDCl₃) δ: 0.64-0.73 (6H, m), 0.96 (9H, t, J=7.1 Hz), 1.31-1.36 (6H, m), 1.54 (3H, s), 3.90-3.99 (1H, m), 5.01-5.10 (1H, m), 5.93 (1H, ddd, J=57.2, 52.6, 3.2 Hz).

### Reference Example 41

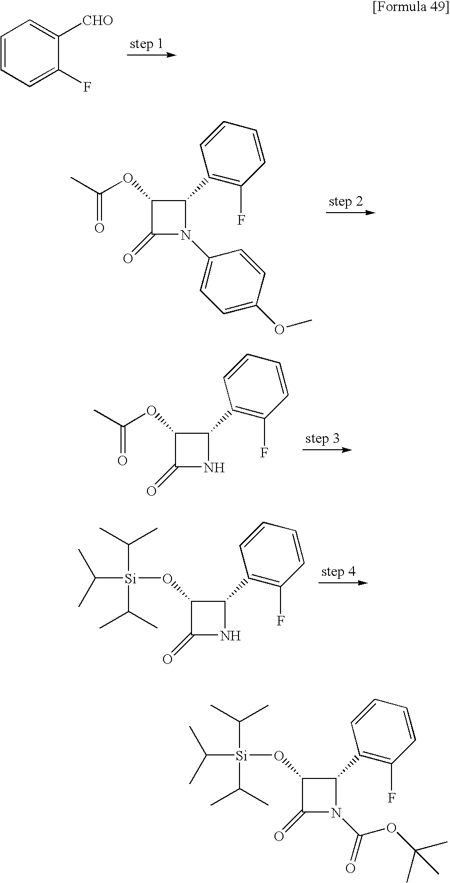

### Step 1: cis-3-Acetoxy-4-(2-fluorophenyl)-1-(4-methoxyphenyl)-2-azetidinone

2-Fluorobenzaldehyde was used as a starting material to perform the same procedure as that of Reference Example 20, Step 1 and thereby to obtain the title compound as colorless solid.

¹H-NMR (400 MHz, CDCl₃) δ: 1.76 (3H, s), 3.76 (3H, s), 5.67 (1H, d, J=4.9 Hz), 6.02 (1H, d, J=4.9 Hz), 6.80-6.84 (2H, m), 7.10-7.13 (2H, m), 7.26-7.37 (4H, m).

### Step 2: cis-3-Acetoxy-4-(2-fluorophenyl)-2-azetidinone

The compound obtained in Step 1 mentioned above was used as a starting material to perform the same procedure as that of Reference Example 18, Step 2 and thereby to obtain the title compound as pale yellow oil.

¹H-NMR (400 MHz, CDCl₃) δ: 1.76 (3H, s), 5.35 (1H, d, J=4.6 Hz), 6.00 (1H, dd, J=4.6, 2.0 Hz), 6.27 (1H, br), 7.03-7.44 (4H, m).

### Step 3: cis-4-(2-Fluorophenyl)-3-[(triisopropylsilyl)oxy]-2-azetidinone

The compound obtained in Step 2 mentioned above was used as a starting material to perform the same procedure as that of Reference Example 20, Step 2 and thereby to obtain the title compound as colorless solid.

¹H-NMR (400 MHz, CDCl₃) δ: 0.87-1.05 (21H, m), 5.18-5.25 (2H, m), 6.00 (1H, brs), 7.00-7.47 (4H, m).

### Step 4: cis-1-(tert-Butoxycarbonyl)-4-(2-fluorophenyl)-3-[(triisopropylsilyl)oxy]-2-azetidinone

The compound obtained in Step 3 mentioned above was used as a starting material to perform the same procedure as that of Reference Example 18, Step 4 and thereby to obtain the title compound as colorless solid.

¹H-NMR (CDCl₃) δ: 0.79-1.02 (21H, m), 1.43 (9H, s), 5.21 (1H, d, J=5.8 Hz), 5.42 (1H, d, J=5.8 Hz), 7.02-7.32 (4H, m).

ESI-MS; m/z: 460 (M+Na)⁺.

### Reference Example 42

### cis-4-(2-Fluorophenyl)-1-(isopropoxycarbonyl)-3-[(triisopropylsilyl)oxy]-2-azetidinone

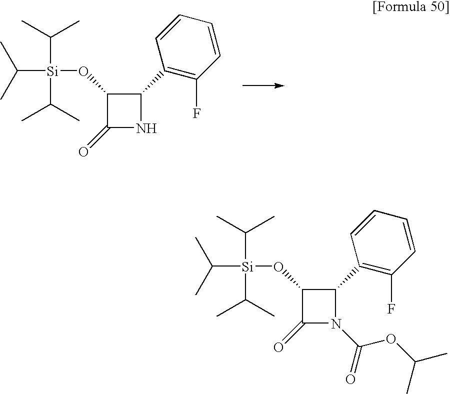

The compound obtained in Reference Example 41, Step 3 was used as a starting material to perform the same procedure as that of Reference Example 21 and thereby to obtain the title compound as colorless oil.

¹H-NMR (CDCl₃) δ: 0.88-1.03 (21H, m), 1.19 (3H, d, J=6.1 Hz), 1.29 (3H, d, J=6.1 Hz), 4.96-5.01 (1H, m), 5.24 (1H, d, J=5.9 Hz), 5.46 (1H, d, J=5.9 Hz), 7.02-7.31 (4H, m).

ESI-MS; m/z: 446 (M+Na)⁺.

### Reference Example 43

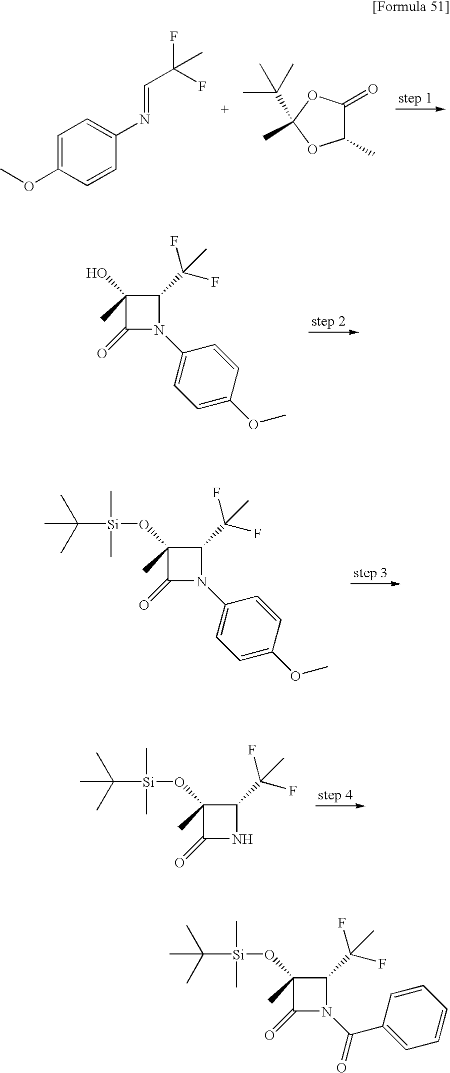

### Step 1: (3R,4R)-4-(1,1-Difluoroethyl)-3-hydroxy-1-(4-methoxyphenyl)-3-methyl-2-azetidinone

The compound obtained in Reference Example 30, Step 1 was used as a starting material to perform the same procedure as that of Reference Example 32, Step 1 and thereby to obtain the title compound as brown oil.

¹H-NMR (CDCl₃) δ: 1.67 (3H, s), 1.79 (3H, t, J=19.1 Hz), 3.06 (1H, brs), 3.80 (3H, s), 4.14-4.19 (1H, m), 6.87-6.90 (2H, m), 7.44-7.47 (2H, m).

FAB-MS; m/z: 272 (M+H)⁺.

### Step 2: (3R,4R)-3-[(tert-Butyldimethylsilyl)oxy]-4-(1,1-difluoroethyl)-1-(4-methoxyphenyl)-3-methyl-2-azetidinone

The compound obtained in Step 1 mentioned above (935 mg) was dissolved in dichloromethane (20 ml), and the solution was added with tert-butyldimethylsilyl trifluoromethanesulfonate (1.58 ml) and 2,6-lutidine (1.61 ml) at 0° C., stirred at room temperature for 3 days, further added with tert-butyldimethylsilyl trifluoromethanesulfonate (1.58 ml) and 2,6-lutidine (1.61 ml), and refluxed overnight by heating. The reaction mixture was added with water, and the layers were separated. The organic layer was successively washed with 10% aqueous citric acid and saturated brine, and dried over anhydrous sodium sulfate, and the solvent was evaporated under reduced pressure. The resulting residue was purified by silica gel chromatography [developing solvent; hexane:ethyl acetate=4:1 (v/v)] to obtain the title compound (703 mg) as colorless oil.

¹H-NMR (CDCl₃) δ: 0.21 (3H, s), 0.31 (3H, s), 0.91 (9H, s), 1.61 (3H, s), 1.74 (3H, t, J=19.1 Hz), 3.79 (3H, s), 4.02-4.07 (1H, m), 6.85-6.90 (2H, m), 7.44-7.48 (2H, m).

EI-MS; m/z: 385 (M⁺).

### Step 3: (3R,4R)-3-[(tert-Butyldimethylsilyl)oxy]-4-(1,1-difluoroethyl)-3-methyl-2-azetidinone

The compound obtained in Step 2 mentioned above was used as a starting material to perform the same procedure as that of Reference Example 18, Step 2 and thereby to obtain the title compound as brown oil.

¹H-NMR (CDCl₃) δ: 0.15 (3H, s), 0.27 (3H, s), 0.89 (9H, s), 1.57 (3H, s), 1.71 (3H, dd, J=19.4, 18.1 Hz), 3.54 (1H, dd, J=17.7, 4.4 Hz), 5.99 (1H, brs).

FAB-MS; m/z: 280 (M+H)⁺.

### Step 4: (3R,4R)-1-Benzoyl-3-[(tert-butyldimethylsilyl)oxy]-4-(1,1-difluoroethyl)-3-methyl-2-azetidinone

The compound obtained in Step 3 mentioned above was used as a starting material to perform the same procedure as that of Reference Example 33 and thereby to obtain the title compound as colorless oil.

¹H-NMR (CDCl₃) δ: 0.23 (3H, s), 0.26 (3H, s), 0.93 (9H, s), 1.56 (3H, s), 1.79 (3H, dd, J=19.1, 18.4 Hz), 4.37 (1H, dd, J=15.2, 6.1 Hz), 7.47-7.56 (3H, m), 7.95-7.98 (2H, m).

### Reference Example 44

### (3R,4R)-4-[(1S)-Fluoroethyl]-1-(isopropoxycarbonyl)-3-[(triisopropylsilyl)oxy]-2-azetidinone

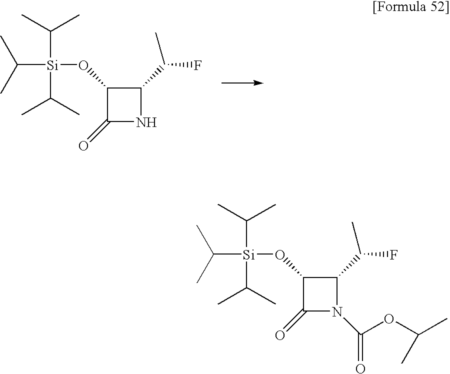

The compound obtained in Reference Example 31, Step 3 was used as a starting material to perform the same procedure as that of Reference Example 21 and thereby to obtain the title compound as colorless oil.

¹H-NMR (CDCl₃) δ: 1.05-1.23 (21H, m), 1.31-1.36 (6H, m), 1.46 (3H, dd, J=24.2, 6.6 Hz), 4.25-4.34 (1H, m), 4.93-5.13 (3H, m).

### Reference Example 45

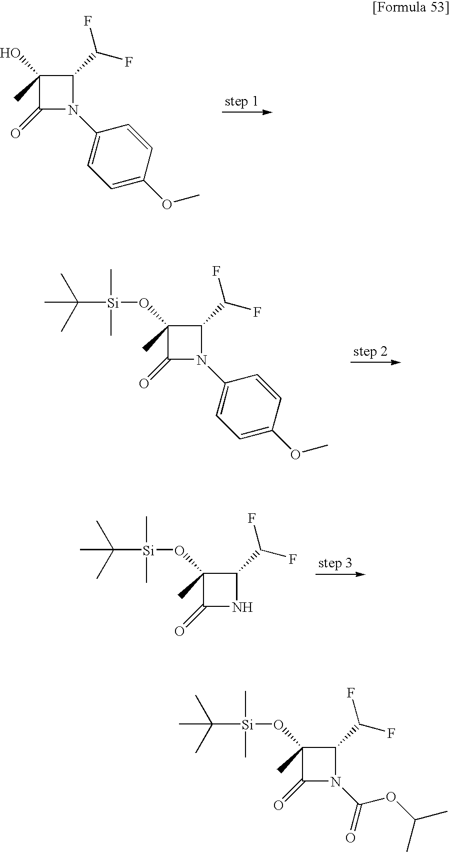

### Step 1: (3R,4R)-3-[(tert-Butyldimethylsilyl)oxy]-4-difluoromethyl-1-(4-methoxyphenyl)-3-methyl-2-azetidinone

The compound obtained in Reference Example 32, Step 1 was used as a starting material to perform the same procedure as that of Reference Example 43, Step 2 and thereby to obtain the title compound as pale yellow amorphous solid.

¹H-NMR (CDCl₃) δ: 0.22 (3H, s), 0.29 (3H, s), 0.91 (9H, s), 1.61 (3H, s), 3.80 (3H, s), 3.97-4.03 (1H, m), 5.81-6.10 (1H, m), 6.86-6.90 (2H, m), 7.43-7.47 (2H, m).

EI-MS; m/z: 371 (M⁺).

### Step 2: (3R,4R)-3-[(tert-Butyldimethylsilyl)oxy]-4-difluoromethyl-3-methyl-2-azetidinone

The compound obtained in Step 1 mentioned above was used as a starting material to perform the same procedure as that of Reference Example 18, Step 2 and thereby to obtain the title compound as brown oil.

¹H-NMR (CDCl₃) δ: 0.18-0.24 (6H, m), 0.89 (9H, s), 1.58 (3H, s), 3.53-3.59 (1H, m), 5.62-5.93 (1H, m), 6.01 (1H, brs).

### Step 3: (3R,4R)-3-[(tert-Butyldimethylsilyl)oxy]-4-difluoromethyl-1-(isopropoxycarbonyl)-3-methyl-2-azetidinone

The compound obtained in Step 2 mentioned above was used as a starting material to perform the same procedure as that of Reference Example 21 and thereby to obtain the title compound as colorless oil.

¹H-NMR (CDCl₃) δ: 0.18 (3H, s), 0.24 (3H, s), 0.88 (9H, s), 1.32-1.36 (6H, m), 1.61 (3H, s), 3.95 (1H, dt, J=8.6, 4.3 Hz), 5.06 (1H, sep, J=6.3 Hz), 5.93 (1H, ddd, J=55.4, 54.0, 4.4 Hz).

### Reference Example 46

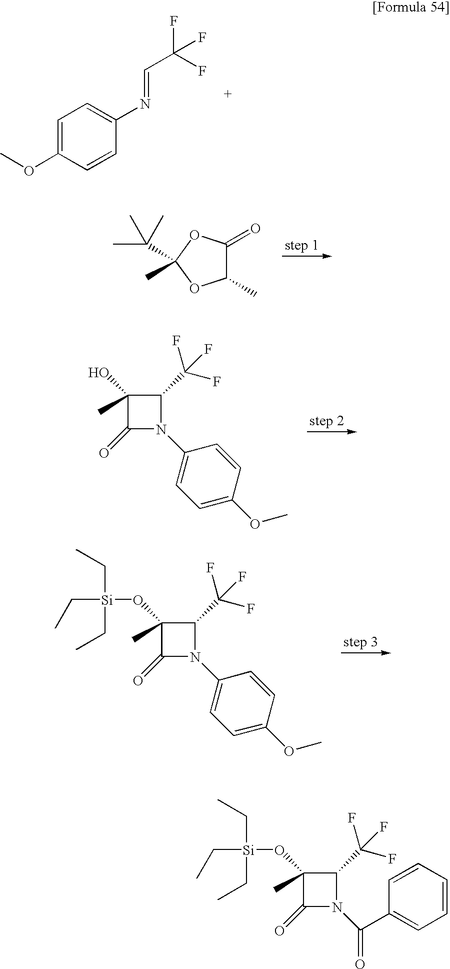

### Step 1: (3R,4R)-3-Hydroxy-1-(4-methoxyphenyl)-3-methyl-4-trifluoromethyl-2-azetidinone

N-(2,2,2-Trifluoroethylidene)-4-methoxyaniline (J. Org. Chem., 1997, 62, 8826) was used as a starting material to perform the same procedure as that of Reference Example 32, Step 1 and thereby to obtain the title compound as pale red solid.

¹H-NMR (CDCl₃) δ: 1.72 (3H, s), 3.37 (1H, brs), 3.80 (3H, s), 4.34 (1H, q, J=5.8 Hz), 6.88-6.92 (2H, m), 7.38-7.42 (2H, m).

### Step 2: (3R,4R)-1-(4-Methoxyphenyl)-3-methyl-3-[(triethylsilyl)oxy]-4-trifluoromethyl-2-azetidinone

The compound obtained in Step 1 mentioned above was used as a starting material to perform the same procedure as that of Reference Example 19, Step 2 and thereby to obtain the title compound as pale yellow oil.

¹H-NMR (CDCl₃) δ: 0.68-0.79 (6H, m), 0.98 (9H, t, J=8.1 Hz), 1.65 (3H, s), 3.79 (3H, s), 4.21 (1H, q, J=5.9 Hz), 6.87-6.90 (2H, m), 7.38-7.41 (2H, m).

### Step 3: (3R,4R)-1-Benzoyl-3-methyl-3-[(triethylsilyl)oxy]-4-trifluoromethyl-2-azetidinone

The compound obtained in Step 2 mentioned above was used as a starting material to perform the same procedure as that of Reference Example 18, Step 2, and then the same procedure as that of Reference Example 33 and thereby to obtain the title compound as colorless oil.

¹H-NMR (CDCl₃) δ: 0.67-0.79 (6H, m), 0.97-1.02 (9H, m), 1.64 (3H, s), 4.58 (1H, q, J=5.9 Hz), 7.48-7.53 (2H, m), 7.61-7.66 (1H, m), 7.96-7.98 (2H, m).

### Reference Example 47

### 3-Methylazetidine hydrochloride

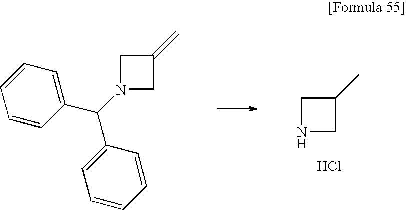

1-Benzhydryl-3-methyleneazetidine (Chem. Pharm. Bull., 1973, 21, 228, 3.45 g) was dissolved in ethanol (50 ml), the solution was added with 1 N hydrochloric acid in ethanol (30 ml) and 20% palladium hydroxide/carbon (1.50 g), and catalytic reduction was performed for 3 days under a hydrogen atmosphere. The catalyst was removed by filtration, and the reaction mixture was concentrated. Then, the resulting residue was added with 1 N aqueous hydrochloric acid and dichloromethane, and the mixture was stirred for 10 minutes. The aqueous layer was evaporated under reduced pressure to obtain the title compound (1.54 g) as pale yellow oil.

¹H-NMR (400 MHz, d₆-DMSO) δ: 1.16 (3H, d, J=6.8 Hz), 2.78-2.86 (1H, m), 3.43-3.58 (2H, m), 3.77-4.01 (2H, m), 9.26 (2H, brs).

### Reference Example 48

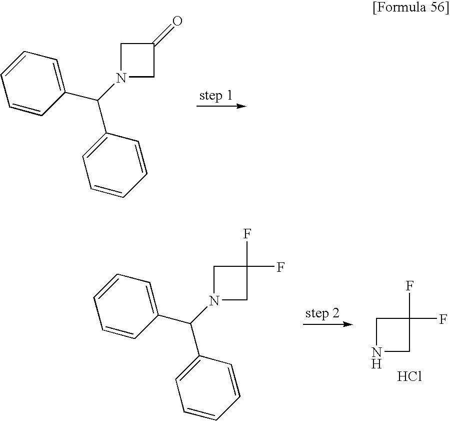

### Step 1: 1-Benzhydryl-3,3-difluoroazetidine

1-Benzhydrylazetidin-3-one was used as a starting material to perform the same procedure as that of Reference Example 18, Step 1 using bis(methoxyethyl)aminosulfur trifluoride instead of diethylaminosulfur trifluoride and thereby to obtain the title compound as pale yellow oil.

¹H-NMR (400 MHz, CDCl₃) δ: 3.51 (4H, t, J=12.2 Hz), 4.44 (1H, s), 7.20 (2H, tt, J=7.3, 1.6 Hz), 7.26-7.30 (4H, m), 7.41-7.44 (4H, m).

ESI-MS; m/z: 260 (M+H)⁺.

### Step 2: 3,3-Difluoroazetidine hydrochloride

The compound obtained in Step 1 mentioned above was used as a starting material to perform the same procedure as that of Reference Example 1 and thereby to obtain the title compound as colorless solid.

¹H-NMR (400 MHz, d₆-DMSO) δ: 4.48 (4H, t, J=11.8 Hz), 10.06 (2H, s).

### Reference Example 49

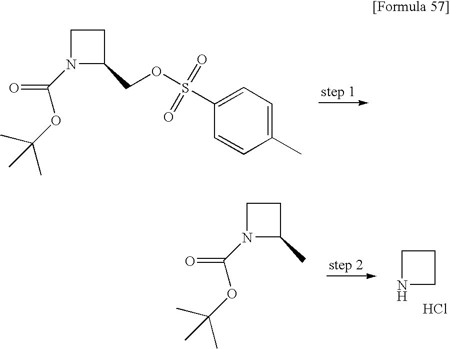

### Step 1: (R)-2-Methylazetidinecarboxylic acid tert-butyl ester

(S)-2-(Toluene-4-sulfonyloxymethyl)azetidinecarboxylic acid tert-butyl ester (J. Med. Chem., 2005, 24, 7637, 6.50 g) was dissolved in tetrahydrofuran (16 ml), and the solution was added dropwise with lithium triethylborohydride (1 mole solution in tetrahydrofuran, 76.2 ml) at 0° C., and stirred at room temperature for 14 hours. The reaction mixture was added with water, and extracted with ethyl acetate. The organic layer was successively washed with 1 N aqueous hydrochloric acid, saturated aqueous sodium hydrogencarbonate and saturated brine, and then dried over anhydrous magnesium sulfate, and the solvent was evaporated under reduced pressure. The resulting residue was purified by silica gel column chromatography [developing solvent; ethyl acetate:n-hexane=1:19 to 1:1 (v/v)] to obtain the title compound (1.48 g) as colorless oil.

¹H-NMR (400 MHz, CDCl₃) δ: 1.37 (3H, d, J=6.13 Hz), 1.44 (9H, s), 1.72-1.81 (1H, m), 2.23-2.32 (1H, m), 3.81 (2H, t, J=7.6 Hz), 4.23-4.32 (1H, m).

### Step 2: (R)-2-Methylazetidine hydrochloride

The compound obtained in Step 1 mentioned above was used as a starting material to perform the same procedure as that of Reference Example 25 and thereby to obtain the title compound as pale yellow oil.

¹H-NMR (400 MHz, CDCl₃) δ: 1.66 (3H, d, J=6.6 Hz), 2.25-2.36 (1H, m), 2.53-2.64 (1H, m), 3.89-4.06 (2H, m), 4.56-4.67 (1H, m), 9.60 (2H, brs).

### Reference Example 50

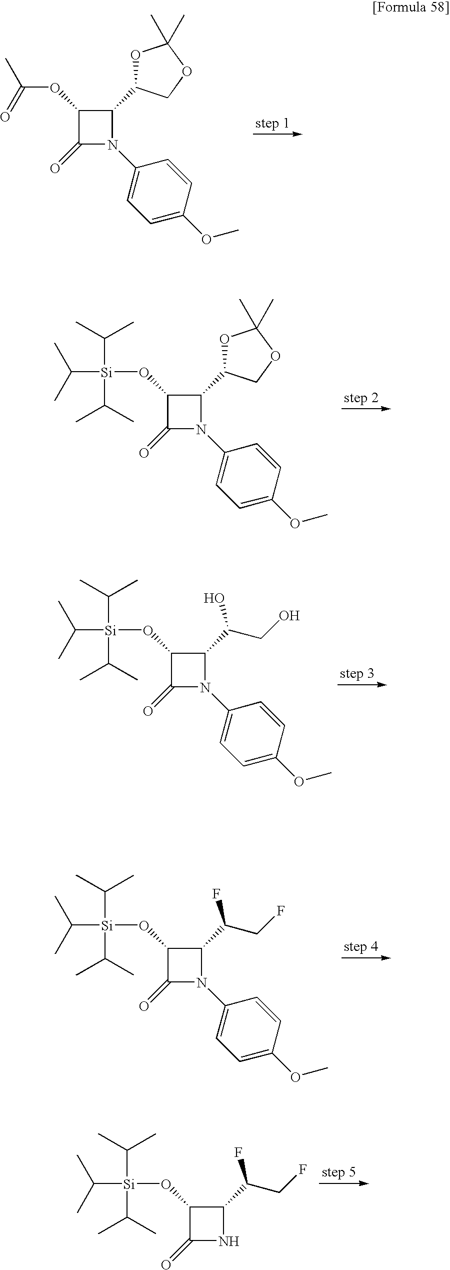

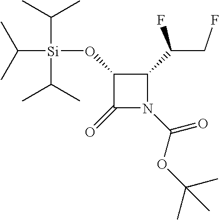

### Step 1: (3R,4S)-4-[(4S)-2,2-Dimethyl-1,3-dioxolan-4-yl]-1-(4-methoxyphenyl)-3-[(triisopropylsilyl)oxy]-2-azetidinone

(3R,4S)-3-Acetoxy-4-[(4S)-2,2-dimethyl-1,3-dioxolan-4-yl]-1-(4-methoxyphenyl)-2-azetidinone (J. Org. Chem., 1988, 53, 4227) was used as a starting material to perform the same procedure as that of Reference Example 20, Step 2 and thereby to obtain the title compound as colorless oil.

¹H-NMR (400 MHz, CDCl₃) δ: 1.10-1.16 (18H, m), 1.18-1.31 (3H, m), 1.54 (3H, s), 1.58 (3H, s), 3.79 (3H, s), 3.83 (1H, dd, J=8.8, 6.1 Hz), 4.16 (1H, dd, J=8.8, 6.1 Hz), 4.28 (1H, dd, J=8.8, 6.8 Hz), 4.37-4.47 (1H, m), 5.05 (1H, d, J=5.4 Hz), 6.83-6.86 (2H, m), 7.62-7.69 (2H, m).

### Step 2: (3R,4S)-4-[(1S)-1,2-Dihydroxyethyl]-1-(4-methoxyphenyl)-3-[(triisopropylsilyl)oxy]-2-azetidinone

The compound obtained in Step 1 mentioned above (25 g) was dissolved in tetrahydrofuran:water (3.5:1, 380 ml), and the solution was added with p-toluenesulfonic acid monohydrate (3.17 g, 16.7 mmol), stirred at 60° C. for 24 hours, and neutralized with sodium hydrogencarbonate under ice cooling. The solvent was evaporated under reduced pressure, and the resulting residue was purified by silica gel chromatography [developing solvent; ethyl acetate:n-hexane=1:8 to 1:2 (v/v)] to obtain the title compound (13.5 g) as pale yellow oil.

¹H-NMR (400 MHz, CDCl₃) δ: 1.14 (18H, t, J=7.1 Hz), 1.19-1.33 (3H, m), 2.07 (1H, dd, J=7.8, 3.9 Hz), 3.04 (1H, d, J=2.9 Hz), 3.63-3.75 (2H, m), 3.78 (3H, s), 4.16-4.23 (1H, m), 4.37 (1H, dd, J=5.2, 2.8 Hz), 5.13 (1H, d, J=5.2 Hz), 6.86 (2H, dt, J=9.8, 2.8 Hz), 7.38 (2H, dt, J=9.8, 2.8 Hz).

### Step 3: (3R,4R)-4-[(1R)-1,2-Difluoroethyl]-1-(4-methoxyphenyl)-3-[(triisopropylsilyl)oxy]-2-azetidinone

The compound obtained in Step 2 mentioned above was used as a starting material to perform the same procedure as that of Reference Example 18, Step 1 using bis(methoxyethyl)aminosulfur trifluoride instead of diethylaminosulfur trifluoride and thereby to obtain the title compound as pale yellow oil.

¹H-NMR (400 MHz, CDCl₃) δ: 0.97-1.29 (21H, m), 3.79 (3H, s), 4.38-4.60 (2H, m), 4.72-4.95 (1H, m), 5.00-5.29 (1H, m), 5.21 (1H, d, J=5.1 Hz), 6.88 (2H, dt, J=9.8, 2.8 Hz), 7.46 (2H, dt, J=9.8, 2.8 Hz).

ESI-MS; m/z: 414 (M+H)⁺.

### Step 4: (3R,4R)-4-[(1R)-1,2-Difluoroethyl]-3-[(triisopropylsilyl)oxy]-2-azetidinone

The compound obtained in Step 3 mentioned above was used as a starting material to perform the same procedure as that of Reference Example 18, Step 2 and thereby to obtain the title compound as colorless solid.

¹H-NMR (400 MHz, CDCl₃) δ: 1.10 (18H, t, J=6.6 Hz), 1.13-1.32 (3H, m), 4.03-4.15 (1H, m), 4.53-5.04 (3H, m), 5.16 (1H, dd, J=4.9, 3.4 Hz), 5.95 (1H, brs).

### Step 5: (3R,4R)-1-(tert-Butoxycarbonyl)-4-[(1R)-1,2-difluoroethyl]-3-[(triisopropylsilyl)oxy]-2-azetidinone

The compound obtained in Step 4 mentioned above was used as a starting material to perform the same procedure as that of Reference Example 18, Step 4 and thereby to obtain the title compound as pale yellow solid.

¹H-NMR (400 MHz, CDCl₃) δ: 1.09 (18H, t, J=6.2 Hz), 1.14-1.29 (3H, m), 1.52 (9H, s), 4.25-4.34 (1H, m), 4.61-4.82 (2H, m), 4.94-5.13 (1H, m), 5.16 (1H, d, J=5.9 Hz).

### Reference Example 51

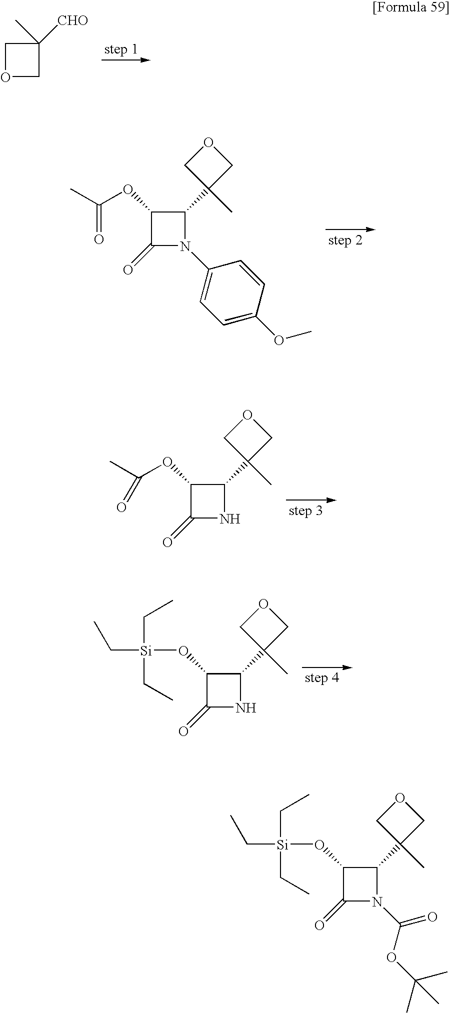

### Step 1: cis-3-Acetoxy-1-(4-methoxyphenyl)-4-(3-methyl-3-oxetanyl)-2-azetidinone

3-Methyloxetane-3-carboxyaldehyde (U.S. Pat. No. 5,354,865) was used as a starting material to perform the same procedure as that of Reference Example 20, Step 1 and thereby to obtain the title compound as colorless solid.

¹H-NMR (400 MHz, CDCl₃) δ: 1.46 (3H, s), 2.13 (3H, s), 3.80 (3H, s), 4.25 (1H, d, J=6.4 Hz), 4.36 (1H, d, J=6.4 Hz), 4.58 (1H, d, J=6.4 Hz), 4.69 (1H, d, J=5.2 Hz), 4.92 (1H, d, J=6.4 Hz), 6.15 (1H, d, J=5.2 Hz), 6.87-6.91 (2H, m), 7.18-7.20 (2H, m).

ESI-MS; m/z: 306 (M+H)⁺.

### Step 2: cis-3-Acetoxy-4-(3-methyl-3-oxetanyl)-2-azetidinone

The compound obtained in Step 1 mentioned above was used as a starting material to perform the same procedure as that of Reference Example 18, Step 2 and thereby to obtain the title compound as pale yellow solid.

¹H-NMR (400 MHz, d₆-DMSO) δ: 1.21 (3H, s), 2.05 (3H, s), 3.78 (1H, d, J=4.9 Hz), 4.17 (2H, dd, J=5.9, 1.6 Hz), 4.43 (1H, d, J=5.9 Hz), 4.58 (1H, d, J=6.3 Hz), 5.83 (1H, dd, J=2.7, 4.9 Hz), 8.94 (1H, brs).

ESI-MS; m/z: 200 (M+H)⁺.

### Step 3: cis-4-(3-Methyl-3-oxetanyl)-3-[(triethylsilyl)oxy]-2-azetidinone

The compound obtained in Step 2 mentioned above was used as a starting material and reacted with triethylsilyl chloride instead of triisopropylsilyl chloride to perform the same procedure as that of Reference Example 20, Step 2 and thereby to obtain the title compound as colorless oil.

¹H-NMR (400 MHz, CDCl₃) δ: 0.62-0.74 (6H, m), 0.96 (9H, t, J=7.9 Hz), 1.41 (3H, s), 3.69 (1H, d, J=4.9 Hz), 4.31 (1H, d, J=6.6 Hz), 4.38 (1H, d, J=6.2 Hz), 4.45 (1H, d, J=6.2 Hz), 4.82 (1H, d, J=6.6 Hz), 4.87 (1H, dd, J=2.8, 4.9 Hz), 6.25 (1H, brs).

### Step 4: cis-1-(tert-Butoxycarbonyl)-4-(3-methyl-3-oxetanyl)-3-[(triethylsilyl)oxy]-2-azetidinone

The compound obtained in Step 3 mentioned above was used as a starting material to perform the same procedure as that of Reference Example 18, Step 4 and thereby to obtain the title compound as colorless oil.

¹H-NMR (400 MHz, CDCl₃) δ: 0.59-0.69 (6H, m), 0.96 (9H, t, J=7.8 Hz), 1.44 (3H, s), 1.51 (9H, s), 4.38 (1H, d, J=6.3 Hz), 4.40 (1H, d, J=6.3 Hz), 4.47 (1H, d, J=6.1 Hz), 4.64 (1H, d, J=6.3 Hz), 4.81 (1H, d, J=6.3 Hz), 4.93 (1H, d, J=6.1 Hz).

ESI-MS; m/z: 372 (M+H)⁺.

### Reference Example 52

### (S)-1-Azetidin-2-ylmethanol oxalate

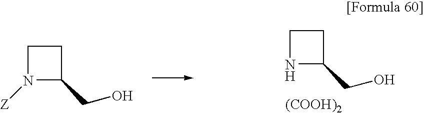

(S)-2-(Hydroxymethyl)-1-azetidinecarboxylic acid benzyl ester (Tetrahedron Lett., 2004, 45, 3607, 1.0 g) was dissolved in ethanol (10 ml), the solution was added with palladium/carbon (200 mg), and catalytic reduction was performed overnight under a hydrogen atmosphere. The catalyst was removed by filtration, and the solution was added with oxalic acid (244 mg), concentrated, and then added with ethanol and isopropanol. The deposited solid was collected by filtration, and dried to obtain the title compound (324 mg) as colorless solid.

¹H-NMR (400 MHz, D₂O) δ: 2.40-2.58 (2H, m), 3.85-3.86 (2H, d, J=4.8 Hz), 3.90-3.96 (1H, m), 4.04-4.11 (1H, m), 4.59-4.65 (1H, m).

FAB-MS; m/z: 88 (M+H)⁺.

### Reference Example 53

### (S)-1-Azetidin-2-ylmethanol fumarate

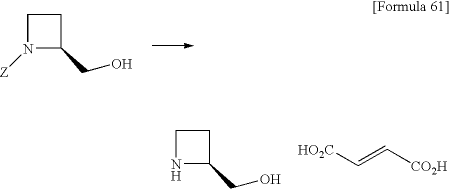

(S)-2-(Hydroxymethyl)-1-azetidinecarboxylic acid benzyl ester (500 mg) was dissolved in ethanol (5 ml), and the solution was added with palladium/carbon (100 mg), and catalytic reduction was performed for 2 hours under a hydrogen atmosphere. The catalyst was removed by filtration, and the solution was added with fumaric acid (262 mg), concentrated, and then added with ethyl acetate and isopropanol. The deposited solid was collected by filtration, and dried to obtain the title compound (389 mg) as colorless solid.

¹H-NMR (400 MHz, D₂O) δ: 2.40-2.58 (2H, m), 3.85-3.86 (2H, d, J=4.8 Hz), 3.90-3.96 (1H, m), 4.04-4.11 (1H, m), 4.59-4.65 (1H, m), 6.71 (2H, s).

FAB-MS; m/z: 88 (M+H)⁺.

### Reference Example 54

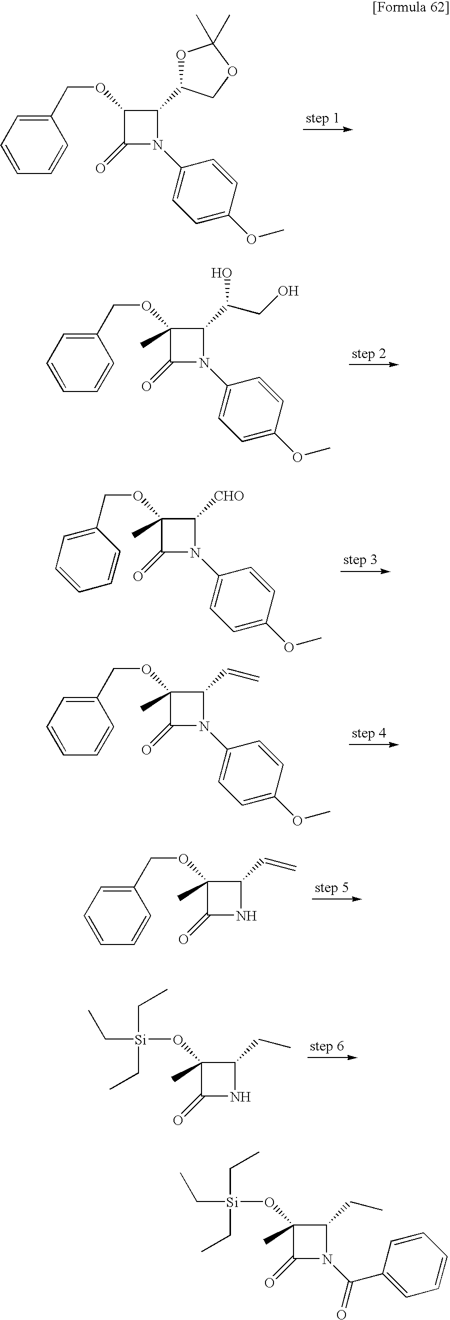

### Step 1: (3R,4S)-3-(Benzyloxy)-4-[(1S)-1,2-dihydroxyethyl]-1-(4-methoxyphenyl)-3-methyl-2-azetidinone

(3R,4S)-3-Benzyloxy-4-[(4S)-2,2-dimethyl-1,3-dioxolan-4-yl]-1-(4-methoxyphenyl)-2-azetidinone (J. Org. Chem., 1998, 53, 4227, 3.75 g) was dissolved in tetrahydrofuran (70 ml), and the solution was added with lithium bis(trimethylsilyl)amide (1 mole solution in tetrahydrofuran, 24.5 ml) at −78° C., and stirred at the same temperature for 2.5 hours. The reaction mixture was added with methyl iodide (2.44 ml), and stirred at room temperature for 2 hours. The reaction mixture was added with saturated aqueous ammonium chloride, and extracted with ethyl acetate, and the organic layer was washed with saturated brine, and then dried over anhydrous sodium sulfate. The solvent was evaporated under reduced pressure, and the resulting residue was dissolved in tetrahydrofuran (80 ml). The solution was added with water (40 ml) and p-toluenesulfonic acid monohydrate (0.37 g), and refluxed for 2 days by heating. The reaction mixture was concentrated under reduced pressure, and the residue was added with saturated aqueous sodium hydrogencarbonate, and extracted with ethyl acetate. The organic layer was washed with saturated brine, and then dried over anhydrous sodium sulfate, and the solvent was evaporated under reduced pressure. The resulting residue was purified by silica gel column chromatography [developing solvent; ethyl acetate:n-hexane=1:3 (v/v)] to obtain the title compound (3.0 g) as brown oil.

¹H-NMR (400 MHz, CDCl₃) δ: 1.72 (3H, s), 2.82 (1H, d, J=4.1 Hz), 3.66-3.71 (2H, m), 3.80 (3H, s), 4.09 (1H, d, J=4.9 Hz), 4.18-4.23 (1H, m), 4.86 (1H, d, J=11.2 Hz), 5.02 (1H, d, J=11.0 Hz), 6.88 (2H, d, J=9.0 Hz), 7.31-7.39 (5H, m), 7.44 (2H, d, J=9.0 Hz).

### Step 2: (3R,4R)-3-(Benzyloxy)-4-formyl-1-(4-methoxyphenyl)-3-methyl-2-azetidinone

The compound obtained in Step 1 mentioned above (3.0 g) was dissolved in ethyl acetate (30 ml), and the solution was added with water (15 ml) and sodium metaperiodate (3.6 g), and stirred at 50° C. for 3 hours. Two of the layers were separated, and then the aqueous layer was extracted with ethyl acetate. The organic layers were combined, washed with saturated aqueous sodium hydrogencarbonate and saturated brine, and then dried over anhydrous sodium sulfate, and the solvent was evaporated under reduced pressure to obtain the title compound (2.4 g) as pale brown solid.

¹H-NMR (400 MHz, CDCl₃) δ: 1.80 (3H, s), 3.80 (3H, s), 4.28 (1H, d, J=3.4 Hz), 4.77 (1H, d, J=11.0 Hz), 4.85 (1H, d, J=11.0 Hz), 6.88-6.91 (2H, m), 7.25-7.34 (7H, m), 9.77 (1H, d, J=3.3 Hz).

### Step 3: (3R,4S)-3-(Benzyloxy)-1-(4-methoxyphenyl)-3-methyl-4-vinyl-2-azetidinone

Methyltriphenylphosphonium bromide (5.4 g) was suspended in tetrahydrofuran (50 ml), and the suspension was added with n-butyl lithium (1.59 mole solution in n-hexane, 9.4 ml) at −78° C., and stirred at room temperature for 20 minutes. The reaction mixture was cooled to −78° C., added with a solution of the compound obtained in Step 2 mentioned above (3.25 g) in tetrahydrofuran (30 ml), and stirred overnight at room temperature. The reaction mixture was added with saturated aqueous ammonium chloride, and extracted with ethyl acetate. The organic layer was washed with saturated brine, and then dried over anhydrous sodium sulfate, and the solvent was evaporated under reduced pressure. The resulting residue was purified by silica gel column chromatography [developing solvent; ethyl acetate:n-hexane=1:5 (v/v)] to obtain the title compound (2.2 g) as yellow solid.

¹H-NMR (400 MHz, CDCl₃) δ: 1.70 (3H, s), 3.79 (3H, s), 4.33 (1H, d, J=8.0 Hz), 4.75 (1H, d, J=11.2 Hz), 4.87 (1H, d, J=11.2 Hz), 5.46 (1H, d, J=11.0 Hz), 5.54 (1H, d, J=17.3 Hz), 6.06 (1H, ddd, J=17.9, 9.8, 7.5 Hz), 6.86 (2H, d, J=9.8 Hz), 7.27-7.42 (7H, m).

### Step 4: (3R,4S)-3-(Benzyloxy)-3-methyl-4-vinyl-2-azetidinone

The compound obtained in Step 3 mentioned above was used as a starting material to perform the same procedure as that of Reference Example 18, Step 2 and thereby to obtain the title compound as brown oil.

¹H-NMR (400 MHz, CDCl₃) δ: 1.66 (3H, s), 3.99 (1H, d, J=8.3 Hz), 4.69 (1H, d, J=11.2 Hz), 4.82 (1H, d, J=11.2 Hz), 5.30-5.39 (2H, m), 5.97-6.10 (2H, m), 7.28-7.36 (5H, m).

### Step 5: (3R,4S)-4-Ethyl-3-methyl-3-[(triethylsilyl)oxy]-2-azetidinone

The compound obtained in Step 4 mentioned above was used as a starting material to perform the same procedure as that of Reference Example 18, Step 3 and thereby to obtain the title compound as colorless oil.

¹H-NMR (400 MHz, CDCl₃) δ: 0.63-0.79 (6H, m), 0.90-1.00 (12H, m), 1.48-1.73 (2H, m), 1.60 (3H, s), 3.26 (1H, t, J=7.1 Hz), 5.90 (1H, brs).

### Step 6: (3R,4S)-1-Benzoyl-4-ethyl-3-methyl-3-[(triethylsilyl)oxy]-2-azetidinone

The compound obtained in Step 5 mentioned above was used as a starting material to perform the same procedure as that of Reference Example 33 and thereby to obtain the title compound as colorless oil.

¹H-NMR (400 MHz, CDCl₃) δ: 0.68-0.81 (6H, m), 0.97-1.05 (12H, m), 1.57 (3H, s), 1.74-1.86 (1H, m), 1.95-2.07 (1H, m), 3.94 (1H, dd, J=9.3, 4.1 Hz), 7.46 (2H, t, J=7.8 Hz), 7.57 (1H, t, J=7.3 Hz), 7.91 (2H, d, J=9.0 Hz).

### Reference Example 55

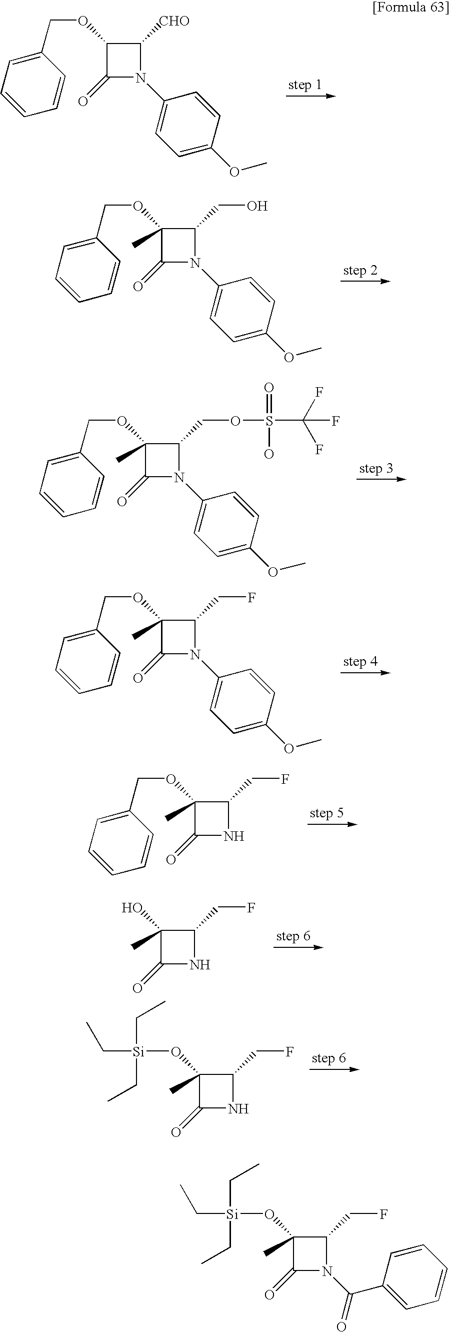

### Step 1: (3R,4S)-3-(Benzyloxy)-4-(hydroxymethyl)-1-(4-methoxyphenyl)-3-methyl-2-azetidinone

The compound obtained in Reference Example 54, Step 2 (3.00 g) was dissolved in a mixed solvent of methanol (30 ml) and tetrahydrofuran (30 ml), and the solution was added with sodium borohydride (291 mg), and stirred at room temperature for 20 minutes. The reaction mixture was added with water and ethyl acetate, and the layers were separated. Organic substances were extracted from the aqueous layer with ethyl acetate. The organic layers were combined, washed with saturated brine, and dried over anhydrous sodium sulfate, and the solvent was evaporated under reduced pressure. The resulting residue was purified by silica gel column chromatography [developing solvent; ethyl acetate:n-hexane=1:2 (v/v)] to obtain the title compound (3.01 g) as colorless solid.

¹H-NMR (400 MHz, CDCl₃) δ: 1.74 (3H, s), 2.52 (1H, t, J=6.9 Hz), 3.79 (3H, s), 3.97-4.09 (3H, m), 4.86 (1H, d, J=10.8 Hz), 5.01 (1H, d, J=11.0 Hz), 6.87-6.91 (2H, m), 7.28-7.41 (7H, m).

### Step 2: (3R,4S)-3-(Benzyloxy)-1-(4-methoxyphenyl)-3-methyl-4-(trifluoromethanesulfonyloxy methyl)-2-azetidinone

The compound obtained in Step 1 mentioned above (100 mg) was dissolved in dichloromethane (5 ml), and the solution was added with trifluoromethanesulfonic acid anhydride (103 μl) and triethylamine (106 μl) at 0° C., returned to room temperature, stirred for 1 hour, further added with trifluoromethanesulfonic acid anhydride (103 μl) and triethylamine (106 μl), and stirred at room temperature for 15 minutes. The reaction mixture was added with water, and the layers were separated. Organic substances were extracted from the aqueous layer with dichloromethane. The organic layers were combined, washed with saturated brine, and dried over anhydrous sodium sulfate, and the solvent was evaporated under reduced pressure. The resulting residue was purified by silica gel column chromatography [developing solvent; ethyl acetate:n-hexane=1:17 (v/v)] to obtain the title compound (126 mg) as pale yellow oil.

¹H-NMR (400 MHz, CDCl₃) δ: 1.74 (3H, s), 3.80 (3H, s), 4.24 (1H, t, J=5.4 Hz), 4.72 (1H, dd, J=11.0, 5.9 Hz), 4.84 (1H, d, J=11.3 Hz), 4.92-4.97 (1H, m), 4.92 (1H, d, J=11.5 Hz), 6.88-6.93 (2H, m), 7.29-7.41 (7H, m).

ESI-MS; m/z: 460 (M+H)⁺.

### Step 3: (3R,4R)-3-(Benzyloxy)-4-fluoromethyl-1-(4-methoxyphenyl)-3-methyl-2-azetidinone

The compound obtained in Step 2 mentioned above (126 mg) was dissolved in acetonitrile (5 ml), and the solution was added with tetra-n-butylammonium fluoride (1 mole solution in tetrahydrofuran, 1.26 ml), and stirred at room temperature for 45 minutes. The reaction mixture was added with water and ethyl acetate, and the layers were separated. Organic substances were extracted from the aqueous layer with ethyl acetate. The organic layers were combined, washed with saturated brine, and dried over anhydrous sodium sulfate, and the solvent was evaporated under reduced pressure. The resulting residue was purified by silica gel column chromatography [developing solvent; ethyl acetate:n-hexane=1:17 (v/v)] to obtain the title compound (73 mg) as colorless amorphous solid.

¹H-NMR (400 MHz, CDCl₃) δ: 1.72 (3H, s), 3.79 (3H, s), 4.18 (1H, ddd, J=14.5, 6.9, 2.9 Hz), 4.65-4.99 (4H, m), 6.87-6.91 (2H, m), 7.26-7.37 (5H, m), 7.46-7.51 (2H, m).

ESI-MS; m/z: 330 (M+H)⁺.

### Step 4: (3R,4R)-3-(Benzyloxy)-4-fluoromethyl-3-methyl-2-azetidinone

The compound obtained in Step 3 mentioned above was used as a starting material to perform the same procedure as that of Reference Example 18, Step 2 and thereby to obtain the title compound as brown oil.

¹H-NMR (400 MHz, CDCl₃) δ: 1.68 (3H, s), 3.76-3.82 (1H, m), 4.49-4.80 (2H, m), 4.71 (1H, d, J=11.3 Hz), 4.84 (1H, d, J=11.3 Hz), 6.35 (1H, brs), 7.26-7.36 (5H, m).

ESI-MS; m/z: 224 (M+H)⁺.

### Step 5: (3R,4R)-4-Fluoromethyl-3-hydroxy-3-methyl-2-azetidinone

The compound obtained in Step 4 mentioned above was used as a starting material to perform the same procedure as that of Reference Example 19, Step 1 and thereby to obtain the title compound as colorless solid.

¹H-NMR (400 MHz, CDCl₃) δ: 1.61 (3H, s), 3.39 (1H, d, J=2.4 Hz), 3.76 (1H, ddd, J=15.9, 6.4, 3.6 Hz), 4.56-4.83 (2H, m), 6.29 (1H, brs).

ESI-MS; m/z: 134 (M+H)⁺.

### Step 6: (3R,4R)-4-Fluoromethyl-3-methyl-3-[(triethylsilyl)oxy]-2-azetidinone

The compound obtained in Step 5 mentioned above was used as a starting material to perform the same procedure as that of Reference Example 19, Step 2 and thereby to obtain the title compound as pale yellow oil.

¹H-NMR (400 MHz, CDCl₃) δ: 0.61-0.76 (6H, m), 0.96 (9H, t, J=8.1 Hz), 1.56 (3H, s), 3.66-3.73 (1H, m), 4.47 (1H, ddd, J=48.4, 9.7, 8.5 Hz), 4.67 (1H, ddd, J=46.6, 10.0, 3.7 Hz), 6.21 (1H, brs).

ESI-MS; m/z: 248 (M+H)⁺.

### Step 7: (3R,4R)-1-(tert-Butoxycarbonyl)-4-fluoromethyl-3-methyl-3-[(triethylsilyl)oxy]-2-azetidinone

The compound obtained in Step 6 mentioned above was used as a starting material to perform the same procedure as that of Reference Example 18, Step 4 and thereby to obtain the title compound as colorless oil.

¹H-NMR (400 MHz, CDCl₃) δ: 0.64-0.74 (6H, m), 0.96 (9H, t, J=7.8 Hz), 1.53 (9H, s), 1.59 (3H, s), 3.91 (1H, ddd, J=15.1, 5.5, 4.2 Hz), 4.59-4.80 (2H, m).

ESI-MS; m/z: 348 (M+H)⁺.

### Reference Example 56

### (3R,4R)-3-[(tert-Butyldimethylsilyl)oxy]-4-(1,1-difluoroethyl)-1-(isopropoxycarbonyl)-3-methyl-2-azetidinone

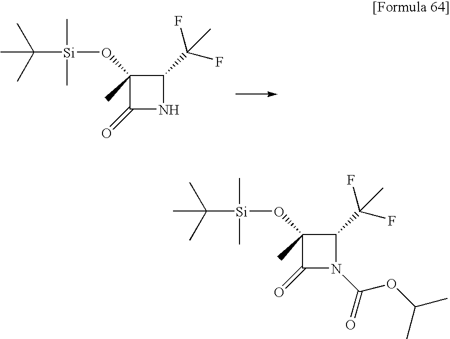

The compound obtained in Reference Example 43, Step 3 was used as a starting material to perform the same procedure as that of Reference Example 21 and thereby to obtain the title compound as pale yellow oil.

¹H-NMR (400 MHz, CDCl₃) δ: 0.17 (3H, s), 0.26 (3H, s), 0.89 (9H, s), 1.32-1.36 (6H, m), 1.61 (3H, s), 1.74 (3H, t, J=18.7 Hz), 3.96 (1H, dd, J=15.0, 4.9 Hz), 5.06 (1H, septet, J=6.2 Hz).

### Reference Example 57

### (3R,4R)-1-(Isopropoxycarbonyl)-3-methyl-3-[(triethylsilyl)oxy]-4-trifluoromethyl-2-azetidinone

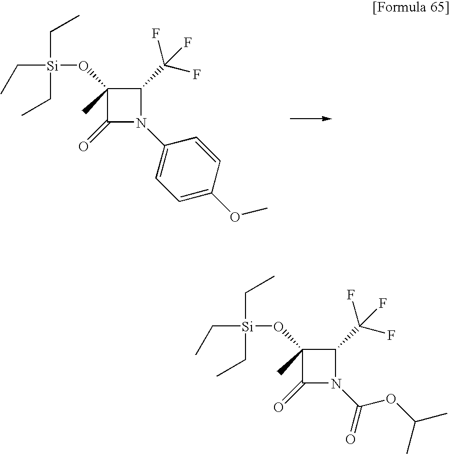

The compound obtained in Reference Example 46, Step 2 was used as a starting material, subjected to the same procedure as that of Reference Example 18, Step 2, and then the same procedure as that of Reference Example 21 and thereby to obtain the title compound as yellow oil.

¹H-NMR (CDCl₃) δ: 0.64-0.77 (6H, m), 0.96 (9H, t, J=8.1 Hz), 1.32-1.36 (6H, m), 1.65 (3H, s), 4.14 (1H, q, J=5.9 Hz), 5.07 (1H, septet, J=6.3 Hz).

### Reference Example 58

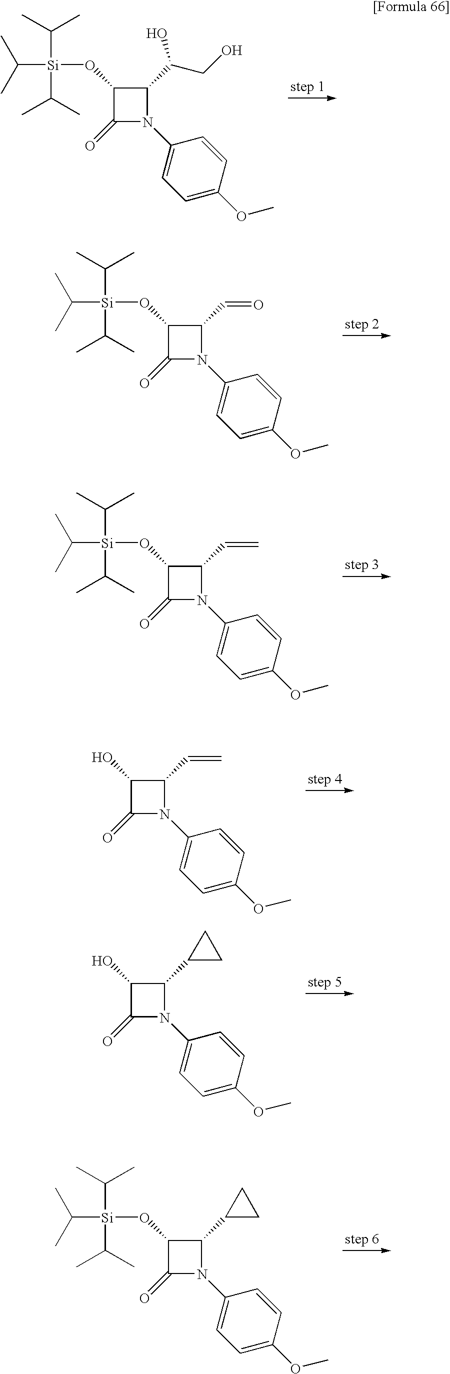

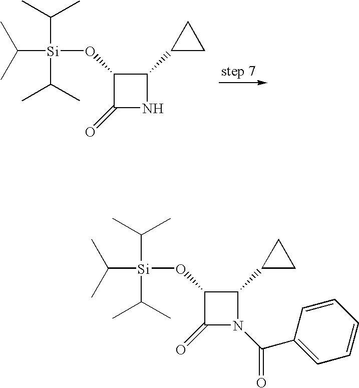

### Step 1: (3R,4R)-4-Formyl-(4-methoxyphenyl)-3-[(triisopropylsilyl)oxy]-2-azetidinone

The compound obtained in Reference Example 50, Step 2 was used as a starting material to perform the same procedure as that of Reference Example 54, Step 2 and thereby to obtain the title compound as pale yellow solid.

¹H-NMR (400 MHz, CDCl₃) δ: 1.07-1.27 (21H, m), 3.78 (3H, s), 4.46 (1H, dd, J=4.2, 5.4 Hz), 5.30 (1H, d, J=5.4 Hz), 6.84-6.88 (2H, m), 7.24-7.28 (2H, m), 9.77 (1H, d, J=4.2 Hz).

### Step 2: (3R,4S)-(4-Methoxyphenyl)-3-[(triisopropylsilyl)oxy]-4-vinyl-2-azetidinone

The compound obtained in Step 1 mentioned above was used as a starting material to perform the same procedure as that of Reference Example 54, Step 3 and thereby to obtain the title compound as colorless solid.

¹H-NMR (400 MHz, CDCl₃) δ: 1.05-1.29 (21H, m), 3.78 (3H, s), 4.57 (1H, dd, J=8.3, 5.2 Hz), 5.10 (1H, d, J=5.2 Hz), 5.44-5.53 (2H, m), 5.93-6.02 (1H, m), 6.84 (2H, d, J=9.1 Hz), 7.38 (2H, d, J=9.1 Hz).

ESI-MS; m/z: 376 (M+H)⁺.

### Step 3: (3R,4S)-3-Hydroxy-(4-methoxyphenyl)-4-vinyl-2-azetidinone

The compound obtained in Step 2 mentioned above (4.80 g) was dissolved in tetrahydrofuran (100 ml), and the solution was added with tetra-n-butylammonium fluoride (1 mole solution in tetrahydrofuran, 19.2 ml) at 0° C., and stirred at 0° C. for 1 hour. The reaction mixture was diluted with water, and extracted with ethyl acetate. The organic layer was washed with saturated brine, and then dried over anhydrous sodium sulfate, and the solvent was evaporated under reduced pressure. The resulting residue was purified by silica gel column chromatography [developing solvent; ethyl acetate:n-hexane=5:9 to 1:1 (v/v)] to obtain the title compound (2.31 g) as colorless solid.

¹H-NMR (400 MHz, CDCl₃) δ: 2.90 (1H, d, J=8.3 Hz), 3.78 (3H, s), 4.70 (1H, td, J=6.1, 0.6 Hz), 5.07 (1H, dd, J=8.2, 5.3 Hz), 5.47-5.58 (2H, m), 5.94-6.03 (1H, m), 6.85 (2H, d, J=8.8 Hz), 7.36 (2H, d, J=8.8 Hz).

ESI-MS; m/z: 220 (M+H)⁺.

### Step 4: (3R,4S)-4-Cyclopropyl-3-hydroxy-(4-methoxyphenyl)-2-azetidinone

The compound obtained in Step 3 mentioned above (1.16 g) was dissolved in 1,2-dichloroethane (50 ml), and the solution was added with diethylzinc (1 mole solution in n-hexane, 62.9 ml) at room temperature under an argon atmosphere, and stirred for 10 minutes. Then, this reaction solution was added dropwise with diiodomethane (10.1 ml), and stirred at room temperature for 7 hours. The reaction mixture was added with saturated aqueous ammonium chloride, and extracted with ethyl acetate. The organic layer was washed with saturated brine, and then dried over anhydrous sodium sulfate, and the solvent was evaporated under reduced pressure. The resulting residue was purified by silica gel column chromatography [developing solvent; ethyl acetate:n-hexane=1:4 to 1:1 (v/v)] to obtain the title compound (0.62 g) as colorless solid.

¹H-NMR (400 MHz, CDCl₃) δ: 0.33-0.39 (1H, m), 0.51-0.57 (1H, m), 0.75-0.88 (2H, m), 1.02-1.11 (1H, m), 3.45 (1H, d, J=7.1 Hz), 3.52 (1H, dd, J=9.1, 5.2 Hz), 3.79 (3H, s), 4.99 (1H, dd, J=6.9, 4.9 Hz), 6.87 (2H, d, J=8.8 Hz), 7.44 (2H, d, J=8.8 Hz).

ESI-MS; m/z: 234 (M+H)⁺.

### Step 5: (3R,4S)-4-Cyclopropyl-(4-methoxyphenyl)-3-[(triisopropylsilyl)oxy]-2-azetidinone

The compound obtained in Step 4 mentioned above was used as a starting material and reacted with triisopropylsilyl chloride instead of triethylsilyl chloride to perform the same procedure as that of Reference Example 19, Step 2 and thereby to obtain the title compound as colorless oil.

¹H-NMR (400 MHz, CDCl₃) δ: 0.27-0.32 (1H, m), 0.49-0.54 (1H, m), 0.67-0.73 (1H, m), 0.76-0.82 (1H, m), 1.11-1.28 (22H, m), 3.45 (1H, dd, J=9.3, 4.9 Hz), 3.79 (3H, s), 5.03 (1H, d, J=4.9 Hz), 6.87 (2H, d, J=9.1 Hz), 7.46 (2H, d, J=9.1 Hz).

ESI-MS; m/z: 390 (M+H)⁺.

### Step 6: (3R,4S)-4-Cyclopropyl-3-[(triisopropylsilyl)oxy]-2-azetidinone

The compound obtained in Step 5 mentioned above was used as a starting material to perform the same procedure as that of Reference Example 18, Step 2 and thereby to obtain the title compound as pale brown oil.

¹H-NMR (400 MHz, CDCl₃) δ: 0.17-0.30 (2H, m), 0.55-0.65 (2H, m), 1.04-1.28 (22H, m), 3.06 (1H, dd, J=8.8, 4.7 Hz), 4.95 (1H, dd, J=4.7, 2.5 Hz), 5.85 (1H, brs).

ESI-MS; m/z: 284 (M+H)⁺.

### Step 7: (3R,4S)-1-Benzoyl-4-cyclopropyl-3-[(triisopropylsilyl)oxy]-2-azetidinone

The compound obtained in Step 6 mentioned above was used as a starting material to perform the same procedure as that of Reference Example 33 and thereby to obtain the title compound as colorless oil.

¹H-NMR (400 MHz, CDCl₃) δ: 0.32-0.37 (1H, m), 0.64-0.73 (2H, m), 0.79-0.84 (1H, m), 1.07-1.28 (22H, m), 3.83 (1H, dd, J=9.3, 6.4 Hz), 5.04 (1H, d, J=6.4 Hz), 7.46 (2H, t, J=7.7 Hz), 7.57 (1H, tt, J=7.5, 1.5 Hz), 7.94-7.96 (2H, m).

ESI-MS; m/z: 410 (M+Na)⁺.

### Reference Example 59

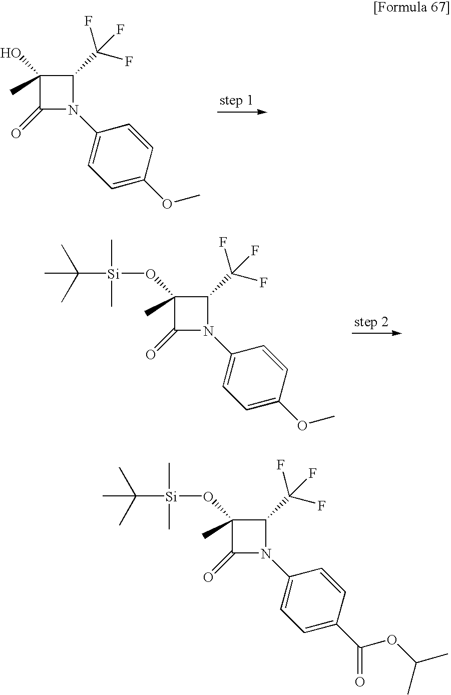

### Step 1: (3R,4R)-3-[(tert-Butyldimethylsilyl)oxy]-1-(4-methoxyphenyl)-3-methyl-4-trifluoromethyl-2-azetidinone

The compound obtained in Reference Example 46, Step 1 was used as a starting material to perform the same procedure as that of Reference Example 43, Step 2 and thereby to obtain the title compound as brown oil.

¹H-NMR (CDCl₃) δ: 0.20 (3H, s), 0.29 (3H, s), 0.90 (9H, s), 1.64 (3H, s), 3.80 (3H, s), 4.21 (1H, q, J=5.9 Hz), 6.87-6.92 (2H, m), 7.38-7.42 (2H, m).

### Step 2: (3R,4R)-3-[(tert-Butyldimethylsilyl)oxy]-1-(isopropoxycarbonyl)-3-methyl-4-trifluoromethyl-2-azetidinone

The compound obtained in Step 1 mentioned above was used as a starting material to perform the same procedure as that of Reference Example 18, Step 2, and then the same procedure as that of Reference Example 21 and thereby to obtain the title compound as pale yellow oil.

¹H-NMR (CDCl₃) δ: 0.16 (3H, s), 0.24 (3H, s), 0.88 (9H, s), 1.33-1.37 (6H, m), 1.64 (3H, s), 4.15 (1H, q, J=5.9 Hz), 5.07 (1H, septet, J=6.3 Hz).

### Reference Example 60

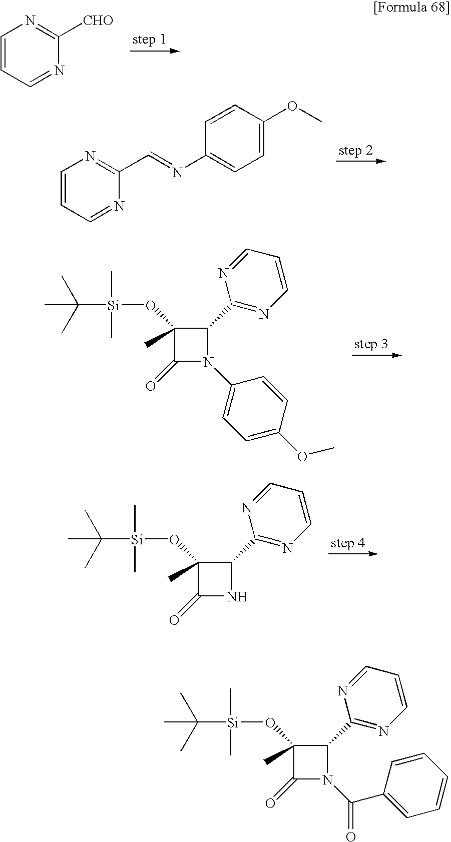

### Step 1: 4-Methoxy-N-[(E)-2-pyrimidinylmethylidene]aniline

The compound obtained in Reference Example 28, Step 2 (15 g) was dissolved in benzene (300 ml), and the solution was added with p-anisidine (13.7 g) and anhydrous sodium sulfate (20 g), and stirred at room temperature for 3 hours. The insoluble matters were removed by filtration, and the filtrate was concentrated. Then, the resulting residue was added with ether, and the deposited solid was collected by filtration, and dried to obtain the title compound (22.5 g) as pale yellow solid.

¹H-NMR (400 MHz, CDCl₃) δ: 3.85 (3H, s), 6.95-6.99 (2H, m), 7.34 (1H, t, J=4.9 Hz), 7.46-7.49 (2H, m), 8.70 (1H, s), 8.92 (2H, d, J=4.9 Hz).

### Step 2: cis-3-[(tert-Butyldimethylsilyl)oxy]-1-(4-methoxyphenyl)-3-methyl-4-(2-pyrimidinyl)-2-azetidinone

A solution of diisopropylamine (18 ml) in tetrahydrofuran (200 ml) was cooled on ice, added dropwise with n-butyl lithium (1.58 mole solution in n-hexane, 80 ml), and stirred for 15 minutes. Then, the reaction mixture was cooled to −78° C., added dropwise with a solution of methyl (S)-(−)-2-[(tert-butyldimethylsilyl)oxy]propionate (J. Org. Chem., 2002, 67, 772, 25.8 g) in tetrahydrofuran (100 ml), and stirred for 30 minutes. The reaction mixture was added dropwise with a solution of the compound obtained in Step 1 mentioned above (24 g) dissolved in tetrahydrofuran (200 ml), and warmed to room temperature overnight. The reaction mixture was added with saturated aqueous ammonium chloride, and extracted with ethyl acetate. The organic layer was successively washed with saturated aqueous sodium hydrogencarbonate, and saturated brine, and then dried over anhydrous sodium sulfate, and the solvent was evaporated under reduced pressure. The resulting residue was purified by silica gel column chromatography [developing solvent; ethyl acetate:n-hexane=1:5 (v/v)], and added with ether/n-hexane, and the deposited solid was collected by filtration, and dried to obtain the title compound (15.2 g) as colorless solid.

¹H-NMR (400 MHz, CDCl₃) δ: 0.05 (3H, s), 0.18 (3H, s), 0.52 (9H, s), 1.55 (3H, s), 1.76 (3H, s), 3.76 (3H, s), 5.06 (1H, s), 6.80-6.83 (2H, m), 7.19-7.29 (3H, m), 8.73 (2H, d, J=4.9 Hz).

### Step 3: (3R,4S)-3-[(tert-Butyldimethylsilyl)oxy]-3-methyl-4-(2-pyrimidinyl)-2-azetidinone

The compound obtained in Step 2 mentioned above was used as a starting material to perform the same procedure as that of Reference Example 18, Step 2, and then the resulting racemate was subjected to optical resolution using an optically active column (CHIRALCEL OD-H, Daicel Chemical Industries) to obtain the title compound as pale yellow solid.

¹H-NMR (400 MHz, CDCl₃) δ: −0.01 (3H, s), 0.14 (3H, s), 0.50 (9H, s), 1.74 (3H, s), 4.74 (1H, s), 6.31 (1H, s), 7.22 (1H, t, J=4.9 Hz), 8.74 (2H, d, J=4.9 Hz).

### Step 4: (3R,4S)-1-Benzoyl-3-[(tert-butyldimethylsilyl)oxy]-3-methyl-4-(2-pyrimidinyl)-2-azetidinone

The compound obtained in Step 3 mentioned above was used as a starting material to perform the same procedure as that of Reference Example 33 and thereby to obtain the title compound as colorless amorphous solid.

¹H-NMR (400 MHz, CDCl₃) δ: 0.06 (3H, s), 0.15 (3H, s), 0.53 (9H, s), 1.79 (3H, s), 5.31 (1H, s), 7.20 (1H, t, J=4.9 Hz), 7.45-7.53 (2H, m), 7.58-7.63 (1H, m), 8.06-8.10 (2H, m), 8.72 (2H, d, J=4.9 Hz).

### Reference Example 61

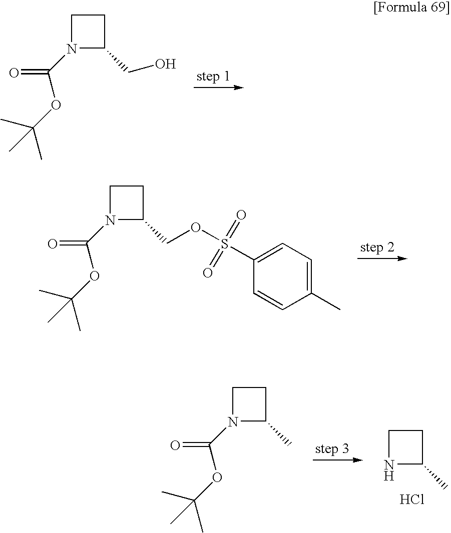

### Step 1: (R)-2-(Toluene-4-sulfonyloxymethyl)azetidinecarboxylic acid tert-butyl ester

(R)-2-(Hydroxymethyl)-1-azetidinecarboxylic acid tert-butyl ester (1.40 g) and pyridine (5.6 ml) were dissolved in dichloromethane (14 ml), and the solution was added with p-toluenesulfonyl chloride (2.14 g) at 0° C., and stirred at room temperature for 24 hours. The reaction mixture was added with water, and extracted with ethyl acetate. The organic layer was successively washed with 1 N aqueous hydrochloric acid and saturated brine, and then dried over anhydrous sodium sulfate, and the solvent was evaporated under reduced pressure. The resulting residue was purified by silica gel column chromatography [developing solvent; ethyl acetate:n-hexane=1:19 to 2:3 (v/v)] to obtain the title compound (2.36 g) as colorless oil.

¹H-NMR (400 MHz, CDCl₃) δ: 1.37 (9H, s), 2.10-2.20 (1H, br), 2.20-2.30 (1H, br), 2.44 (3H, s), 3.72-3.83 (2H, m), 4.13 (1H, dd, J=10.2, 2.8 Hz), 4.22-4.29 (1H, br), 4.30-4.36 (1H, br), 7.34 (2H, d, J=8.1 Hz), 7.80 (2H, d, J=8.1 Hz).

### Step 2: (S)-2-Methylazetidinecarboxylic acid tert-butyl ester

The compound obtained in Step 1 mentioned above was used as a starting material to perform the same procedure as that of Reference Example 49, Step 1 and thereby to obtain the title compound as colorless oil.

¹H-NMR (400 MHz, CDCl₃) δ: 1.37 (3H, d, J=6.1 Hz), 1.44 (9H, d, J=1.0 Hz), 1.71-1.81 (1H, m), 2.23-2.32 (1H, m), 3.81 (2H, t, J=7.6 Hz), 4.23-4.32 (1H, m).

### Step 3: (S)-2-Methylazetidine hydrochloride

The compound obtained in Step 2 mentioned above was used as a starting material to perform the same procedure as that of Reference Example 25 and thereby to obtain the title compound as colorless oil.

¹H-NMR (400 MHz, CDCl₃) δ: 1.67 (3H, d, J=6.9 Hz), 2.26-2.37 (1H, m), 2.54-2.64 (1H, m), 3.91-4.08 (2H, m), 4.58-4.69 (1H, m), 9.59 (2H, brs).

### Reference Example 62

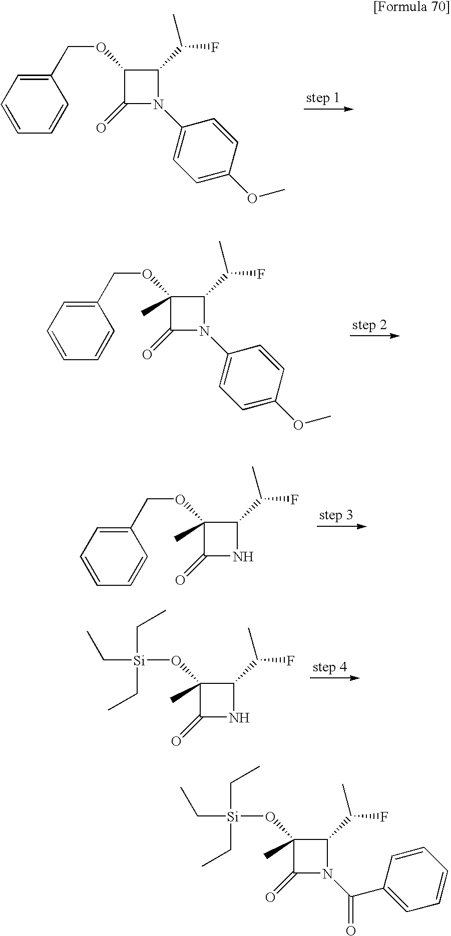

### Step 1: (3R,4R)-3-(Benzyloxy)-4-[(1S)-1-fluoroethyl]-1-(4-methoxyphenyl)-3-methyl-2-azetidin one

The compound obtained in Reference Example 31, Step 1 (1.6 g) was dissolved in tetrahydrofuran (40 ml), and the solution was added with lithium bis(trimethylsilyl)amide (1 mole solution in tetrahydrofuran, 12.2 ml) at −78° C., and stirred at the same temperature for 2.5 hours. The reaction mixture was added with methyl iodide (1.2 ml), and stirred at 0° C. for 1 hour. The reaction mixture was added with saturated aqueous ammonium chloride, and extracted with ethyl acetate. The organic layer was washed with saturated brine, and then dried over anhydrous sodium sulfate, and the solvent was evaporated under reduced pressure. The resulting residue was purified by silica gel column chromatography [developing solvent; ethyl acetate:n-hexane=1:8 (v/v)] to obtain the title compound (1.2 g) as yellow solid.

¹H-NMR (400 MHz, CDCl₃) δ: 1.35 (3H, dd, J=25.6, 6.8 Hz), 1.72 (3H, s), 3.80 (3H, s), 4.23 (1H, d, J=17.6 Hz), 4.81 (1H, d, J=12.0 Hz), 4.91 (1H, d, J=11.5 Hz), 5.00-5.19 (1H, m), 6.89 (2H, d, J=8.8 Hz), 7.29-7.38 (5H, m), 7.51 (2H, d, J=8.5 Hz).

### Step 2: (3R,4R)-3-(Benzyloxy)-4-[(1S)-1-fluoroethyl]-3-methyl-2-azetidinone

The compound obtained in Step 1 mentioned above was used as a starting material to perform the same procedure as that of Reference Example 18, Step 2 and thereby to obtain the title compound as orange oil.

¹H-NMR (400 MHz, CDCl₃) δ: 1.38 (3H, dd, J=24.9, 6.3 Hz), 1.69 (3H, s), 3.64 (1H, dd, J=12.4, 5.9 Hz), 4.74 (1H, d, J=11.2 Hz), 4.84 (1H, d, J=11.5 Hz), 4.85-5.04 (1H, m), 5.83 (1H, brs), 7.27-7.36 (5H, m).

### Step 3: (3R,4R)-4-[(1S)-1-Fluoroethyl]-3-methyl-3-[(triethylsilyl)oxy]-2-azetidinone

The compound obtained in Step 2 mentioned above was used as a starting material to perform the same procedure as that of Reference Example 18, Step 3 using triethylsilyl chloride instead of triisopropylsilyl chloride and thereby to obtain the title compound as colorless oil.

¹H-NMR (400 MHz, CDCl₃) δ: 0.65-0.74 (6H, m), 0.97 (9H, t, J=7.8 Hz), 1.37 (3H, dd, J=24.6, 6.6 Hz), 1.57 (3H, s), 3.51 (1H, dd, J=12.7, 5.9 Hz), 4.74-4.92 (1H, m), 5.93 (1H, brs).

### Step 4: (3R,4R)-1-Benzoyl-4-[(1S)-1-fluoroethyl]-3-methyl-3-[(triethylsilyl)oxy]-2-azetidinone

The compound obtained in Step 3 mentioned above was used as a starting material to perform the same procedure as that of Reference Example 33 and thereby to obtain the title compound as colorless oil.

¹H-NMR (400 MHz, CDCl₃) δ: 0.69-0.80 (6H, m), 1.00 (9H, t, J=7.8 Hz), 1.48 (3H, dd, J=23.9, 7.6 Hz), 1.60 (3H, s), 4.15 (1H, dd, J=16.8, 4.6 Hz), 5.03-5.21 (1H, m), 7.48 (2H, t, J=7.6 Hz), 7.60 (1H, t, J=7.8 Hz), 7.93 (2H, d, J=8.3 Hz).

### Reference Example 63

### (3R,4R)-4-Fluoromethyl-1-(isopropoxycarbonyl)-3-methyl-3-[(triethylsilyl)oxy]-2-azetidinone

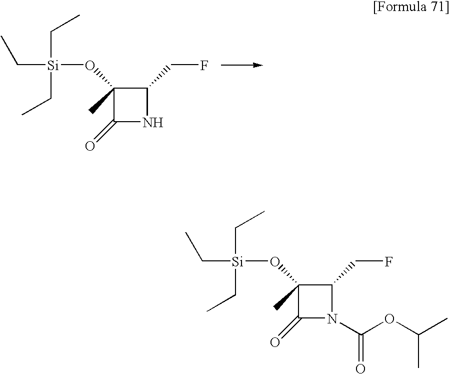

The compound obtained in Reference Example 55, Step 6 was used as a starting material to perform the same procedure as that of Reference Example 21 and thereby to obtain the title compound as pale yellow oil.

¹H-NMR (400 MHz, CDCl₃) δ: 0.64-0.73 (6H, m), 0.96 (9H, t, J=7.8 Hz), 1.33 (3H, d, J=4.4 Hz), 1.34 (3H, d, J=4.4 Hz), 1.59 (3H, s), 3.94 (1H, ddd, J=15.0, 5.0, 4.4 Hz), 4.61-4.82 (2H, m), 5.05 (1H, septet, J=6.1 Hz).

### Reference Example 64

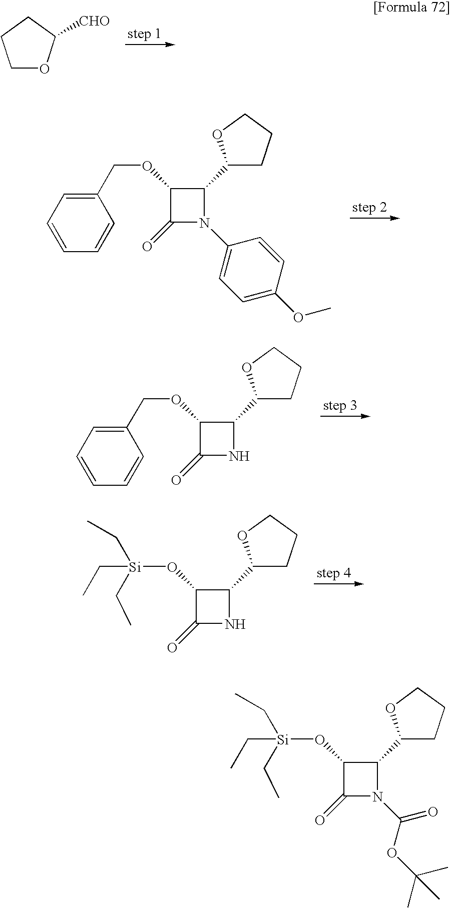

### Step 1: (3R,4S)-3-(Benzyloxy)-1-(4-methoxyphenyl)-4-[(2R)-tetrahydrofuran-2-yl]-2-azetidinone

(2R)-Tetrahydrofuran-2-carboxyaldehyde (Can. J. Chem., 1983, 61, 1383) was used as a starting material to perform the same procedure as that of Reference Example 20, Step 1 using benzyloxyacetyl chloride instead of acetoxyacetyl chloride and thereby to obtain the title compound as colorless solid.

¹H-NMR (400 MHz, CDCl₃) δ: 1.58-1.68 (1H, m), 1.85-1.94 (2H, m), 2.22-2.33 (1H, m), 3.72-3.79 (1H, m), 3.78 (3H, s), 3.89-3.96 (1H, m), 4.09-4.24 (2H, m), 4.74 (1H, d, J=5.4 Hz), 4.77 (1H, d, J=12.0 Hz), 4.97 (1H, d, J=11.7 Hz), 6.85 (2H, d, J=9.0 Hz), 7.29-7.40 (5H, m), 7.64 (2H, d, J=9.8 Hz).

### Step 2: (3R,4S)-3-(Benzyloxy)-4-[(2R)-tetrahydrofuran-2-yl]-2-azetidinone

The compound obtained in Step 1 mentioned above was used as a starting material to perform the same procedure as that of Reference Example 18, Step 2 and thereby to obtain the title compound as brown solid.

¹H-NMR (400 MHz, CDCl₃) δ: 1.46-1.56 (1H, m), 1.87-1.97 (2H, m), 2.09-2.20 (1H, m), 3.58 (1H, dd, J=9.0, 5.1 Hz), 3.71-3.87 (2H, m), 4.03-4.11 (1H, m), 4.66-4.72 (2H, m), 4.92 (1H, d, J=11.7 Hz), 6.23 (1H, brs), 7.27-7.38 (5H, m).

### Step 3: (3R,4S)-4-[(2R)-Tetrahydrofuran-2-yl]-3-[(triethylsilyl)oxy]-2-azetidinone

The compound obtained in Step 2 mentioned above was used as a starting material to perform the same procedure as that of Reference Example 18, Step 3 using triethylsilyl chloride instead of triisopropylsilyl chloride and thereby to obtain the title compound as colorless oil.

¹H-NMR (400 MHz, CDCl₃) δ: 0.64-0.73 (6H, m), 0.98 (9H, t, J=8.1 Hz), 1.43-1.56 (1H, m), 1.87-1.97 (2H, m), 2.03-2.15 (1H, m), 3.51 (1H, dd, J=9.2, 5.0 Hz), 3.70-3.78 (1H, m), 3.79-3.86 (1H, m), 3.94-4.02 (1H, m), 4.85 (1H, dd, J=5.1, 2.0 Hz), 6.16 (1H, brs).

### Step 4: (3R,4S)-1-(tert-Butoxycarbonyl)-4-[(2R)-tetrahydrofuran-2-yl]-3-[(triethylsilyl)oxy]-2-azetidinone

The compound obtained in Step 3 mentioned above was used as a starting material to perform the same procedure as that of Reference Example 18, Step 4 and thereby to obtain the title compound as colorless oil.

¹H-NMR (400 MHz, CDCl₃) δ: 0.65-0.73 (6H, m), 0.98 (9H, t, J=7.6 Hz), 1.52 (9H, s), 1.71-1.82 (1H, m), 1.84-1.95 (2H, m), 2.04-2.13 (1H, m), 3.75-3.81 (1H, m), 3.85-3.91 (1H, m), 4.02-4.07 (1H, m), 4.15 (1H, dd, J=13.9, 6.6 Hz), 4.85 (1H, d, J=6.1 Hz).

### Example 1

### (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3S)-3-(tert-butoxycarbonylamino)-2-hydroxy-3-(2-pyridyl)propionate

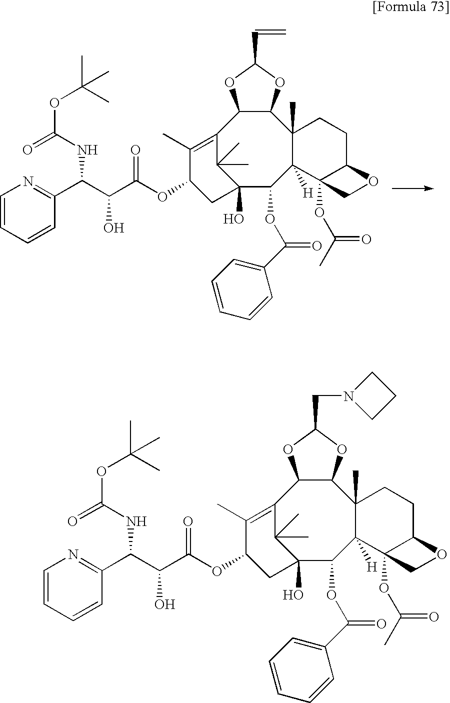

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-2-hydroxy-3-(2-pyridyl)propionate (Bioorg. Med. Chem. Lett., 2003, 13, 185, 50 mg) was dissolved in tetrahydrofuran (1 ml), acetone (1 ml) and water (1 ml), and the solution was added with osmium tetroxide (0.76 mg) and N-methylmorpholine N-oxide (36.3 mg) at room temperature, and stirred for 6 hours. The reaction mixture was added with 10% aqueous sodium thiosulfate, and extracted with ethyl acetate. The organic layer was successively washed with saturated aqueous sodium hydrogencarbonate and saturated brine, and then dried over anhydrous sodium sulfate. The solvent was evaporated under reduced pressure, and then the residue was dissolved in tetrahydrofuran (1 ml), methanol (1 ml) and water (1 ml), and the solution was added with sodium metaperiodate (154 mg) at room temperature, and stirred for 2.5 hours. The reaction mixture was added with water and saturated brine, and extracted with ethyl acetate. The organic layer was washed with saturated aqueous sodium hydrogencarbonate, and then dried over anhydrous sodium sulfate. The solvent was evaporated under reduced pressure, and the resulting residue was dissolved in methanol (2 ml). The solution was added with triethylamine (0.0418 ml) and azetidine hydrochloride (29 mg) at room temperature, and stirred for 24 hours. The reaction mixture was concentrated, and the residue was dissolved in methanol (2 ml). The solution was added with sodium triacetoxyborohydride (67 mg), and stirred at room temperature for 2 hours. The reaction mixture was added with saturated aqueous sodium hydrogencarbonate, and extracted with ethyl acetate. The organic layer was washed with saturated brine, and then dried over anhydrous sodium sulfate. The solvent was evaporated under reduced pressure, and then the residue was purified by thin layer silica gel column chromatography [developing solvent; chloroform:methanol=20:1 (v/v)] to obtain the title compound (21.4 mg) as white amorphous solid.

¹H-NMR (400 MHz, CDCl₃) δ: 1.22-2.24 (9H, m), 1.25 (3H, s), 1.44 (9H, s), 1.49 (3H, s), 1.60 (3H, s), 1.70 (3H, s), 2.31-2.37 (1H, m), 2.34 (3H, s), 2.75 (1H, dd, J=12.7, 5.9 Hz), 2.84 (1H, dd, J=12.7, 4.2 Hz), 2.92 (1H, d, J=5.1 Hz), 3.36-3.40 (4H, m), 4.12 (1H, d, J=7.1 Hz), 4.22 (1H, d, J=8.5 Hz), 4.33 (1H, d, J=8.5 Hz), 4.86-4.92 (3H, m), 5.20 (1H, d, J=7.3 Hz), 5.35 (1H, d, J=8.3 Hz), 5.94-5.99 (2H, m), 6.08 (1H, t, J=8.2 Hz), 7.21-7.25 (1H, m), 7.41-7.49 (3H, m), 7.60 (1H, t, J=7.4 Hz), 7.72 (1H, dt, J=7.7, 1.7 Hz), 8.13 (2H, d, J=7.7 Hz), 8.54 (1H, d, J=4.4 Hz).

MS (FAB)m/z: 876 (M+H)⁺.

### Example 2

### (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3S)-3-(tert-butoxycarbonylamino)-3-(3-fluoro-2-pyridyl)-2-hydroxypropionate

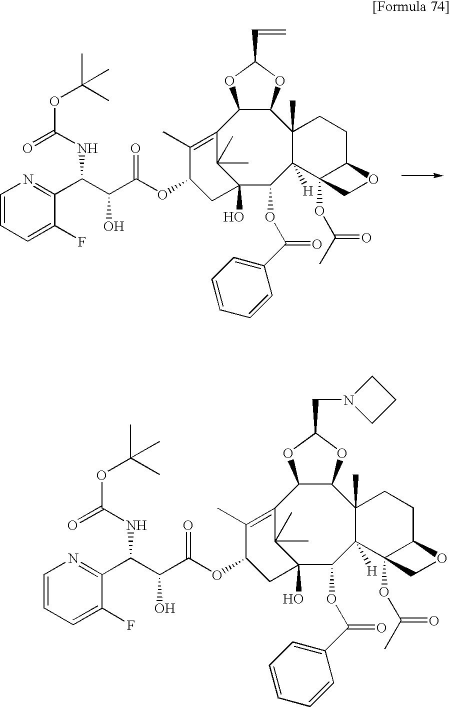

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-3-(3-fluoro-2-pyridyl)-2-hydroxypropionate (Bioorg. Med. Chem. Lett., 2004, 14, 3209) was used as a starting material to perform the same procedure as that of Example 1 and thereby to obtain the title compound as white amorphous solid.

¹H-NMR (400 MHz, CDCl₃) δ: 1.25-2.18 (9H, m), 1.28 (3H, s), 1.40 (9H, s), 1.50 (3H, s), 1.62 (3H, s), 1.78 (3H, s), 2.34 (3H, s), 2.42-2.48 (1H, m), 2.76 (1H, dd, J=12.5, 5.7 Hz), 2.84 (1H, dd, J=12.5, 4.4 Hz), 2.93 (1H, d, J=5.1 Hz), 3.36-3.41 (4H, m), 4.13 (1H, d, J=7.1 Hz), 4.23 (1H, d, J=8.3 Hz), 4.33 (1H, d, J=8.3 Hz), 4.68 (1H, d, J=2.7 Hz), 4.88 (1H, t, J=5.1 Hz), 4.91 (1H, s), 5.22 (1H, d, J=6.8 Hz), 5.65 (1H, d, J=8.3 Hz), 5.99 (1H, d, J=5.1 Hz), 6.08 (1H, t, J=8.5 Hz), 6.21 (1H, d, J=8.8 Hz), 7.28-7.33 (1H, m), 7.42-7.49 (3H, m), 7.60 (1H, t, J=7.4 Hz), 8.12-8.14 (2H, m), 8.40 (1H, d, J=4.6 Hz).

MS (FAB)m/z: 894 (M+H)⁺.

### Example 3

### (1S,2S,3R,4S,5R,8R,9S,10R,13S)-4-Acetoxy-2-(benzoyloxy)-5,20-epoxy-9,10-[(1S)-2-(3-fluoroazetidino)ethylidenedioxy]-1-hydroxytax-11-en-13-yl

### (2R,3S)-3-(tert-butoxycarbonylamino)-2-hydroxy-3-(2-pyridyl)propionate

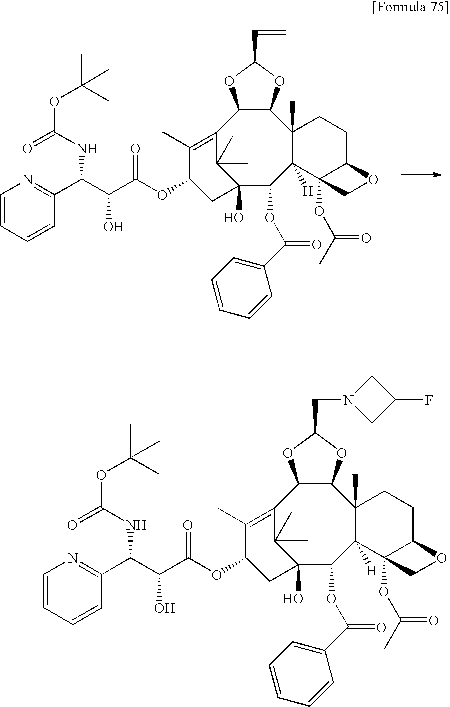

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-2-hydroxy-3-(2-pyridyl)propionate and the compound obtained in Reference Example 24, Step 2 were used as starting materials to perform the same procedure as that of Example 1 and thereby to obtain the title compound as white amorphous solid.

¹H-NMR (400 MHz, CDCl₃) δ 1.25-2.13 (7H, m), 1.26 (3H, s), 1.44 (9H, s), 1.49 (3H, s), 1.60 (3H, s), 1.71 (3H, s), 2.31-2.38 (1H, m), 2.35 (3H, s), 2.82 (1H, dd, J=12.3, 5.5 Hz), 2.89-2.93 (2H, m), 3.27-3.39 (2H, m), 3.77-3.82 (2H, m), 4.13 (1H, d, J=7.1 Hz), 4.21 (1H, d, J=8.3 Hz), 4.33 (1H, d, J=8.3 Hz), 4.86-4.92 (3H, m), 5.07-5.24 (2H, m), 5.35 (1H, d, J=9.3 Hz), 5.94-5.98 (2H, m), 6.09 (1H, t, J=7.8 Hz), 7.22-7.25 (1H, m), 7.41-7.49 (3H, m), 7.60 (1H, t, J=7.6 Hz), 7.72 (1H, t, J=7.6 Hz), 8.13 (2H, d, J=7.6 Hz), 8.54 (1H, d, J=3.9 Hz).

MS (FAB)m/z: 894 (M+H)⁺.

### Example 4

### (1S,2S,3R,4S,5R,8R,9S,10R,13S)-4-(Acetoxy)-2-(benzoyloxy)-5,20-epoxy-9,10-[(1S)-2-(3-fluoroazetidino)ethylidenedioxy]-1-hydroxytax-11-en-13-yl

### (2R,3S)-3-(tert-butoxycarbonylamino)-3-(3-fluoro-2-pyridyl)-2-hydroxypropionate

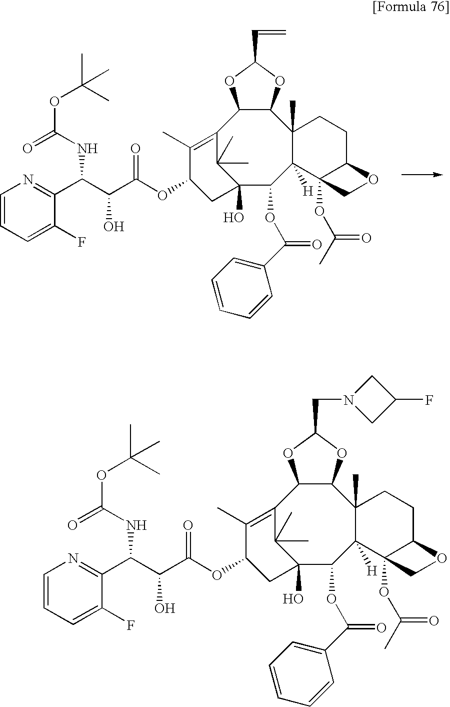

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-3-(3-fluoro-2-pyridyl)-2-hydroxypropionate and the compound obtained in Reference Example 24, Step 2 were used as starting materials to perform the same procedure as that of Example 1 and thereby to obtain the title compound as white amorphous solid.

¹H-NMR (400 MHz, CDCl₃) δ: 1.26-2.16 (7H, m), 1.29 (3H, s), 1.40 (9H, s), 1.50 (3H, s), 1.61 (3H, s), 1.78 (3H, s), 2.34 (3H, s), 2.42-2.48 (1H, m), 2.82 (1H, dd, J=12.3, 5.7 Hz), 2.92 (2H, dd, J=12.3, 4.4 Hz), 3.27-3.39 (2H, m), 3.76-3.84 (2H, m), 4.14 (1H, d, J=7.1 Hz), 4.23 (1H, d, J=8.3 Hz), 4.33 (1H, d, J=8.3 Hz), 4.67 (1H, d, J=2.2 Hz), 4.87-4.92 (2H, m), 5.07-5.23 (2H, m), 5.64 (1H, d, J=8.8 Hz), 5.99 (1H, d, J=4.9 Hz), 6.09 (1H, t, J=8.3 Hz), 6.21 (1H, d, J=7.8 Hz), 7.28-7.33 (1H, m), 7.43-7.49 (3H, m), 7.60 (1H, t, J=7.6 Hz), 8.14 (2H, d, J=7.6 Hz), 8.40 (1H, d, J=4.2 Hz).

MS (FAB)m/z: 912 (M+H)⁺.

### Example 5

### (1S,2S,3R,4S,5R,8R,9S,10R,13S)-4-(Acetoxy)-2-(benzoyloxy)-5,20-epoxy-1-hydroxy-9,10-[(1S)-2-(3-hydroxyazetidino)ethylidenedioxy]tax-11-en-13-yl

### (2R,3S)-3-(tert-butoxycarbonylamino)-2-hydroxy-3-(2-pyridyl)propionate

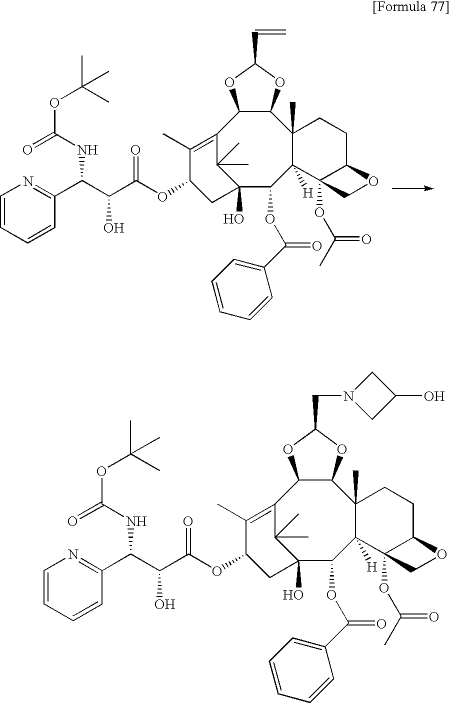

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-2-hydroxy-3-(2-pyridyl)propionate and 3-azetidinol hydrochloride was used as a starting material to perform the same procedure as that of Example 1 and thereby to obtain the title compound as white amorphous solid.

¹H-NMR (400 MHz, CDCl₃) δ 1.24-2.13 (8H, m), 1.26 (3H, s), 1.44 (9H, s), 1.49 (3H, s), 1.60 (3H, s), 1.71 (3H, s), 2.31-2.37 (1H, m), 2.35 (3H, s), 2.76-2.82 (1H, m), 2.86-2.93 (2H, m), 3.07-3.14 (2H, m), 3.74-3.80 (2H, m), 4.12 (1H, d, J=7.1 Hz), 4.21 (1H, d, J=8.3 Hz), 4.32 (1H, d, J=8.3 Hz), 4.46-4.50 (1H, m), 4.86-4.92 (3H, m), 5.20 (1H, d, J=6.1 Hz), 5.35 (1H, d, J=9.0 Hz), 5.98 (2H, s), 6.09 (1H, s), 7.21-7.25 (1H, m), 7.41-7.50 (3H, m), 7.60 (1H, t, J=7.6 Hz), 7.72 (1H, t, J=7.6 Hz), 8.13 (2H, d, J=7.6 Hz), 8.54 (1H, s).

FAB-MS; m/z: 892 (M+H)⁺.

### Example 6

### (1S,2S,3R,4S,5R,8R,9S,10R,13S)-4-(Acetoxy)-2-(benzoyloxy)-5,20-epoxy-1-hydroxy-9,10-[(1S)-2-(3-hydroxyazetidino)ethylidenedioxy]tax-11-en-13-yl

### (2R,3S)-3-(tert-butoxycarbonylamino)-3-(3-fluoro-2-pyridyl)-2-hydroxypropionate

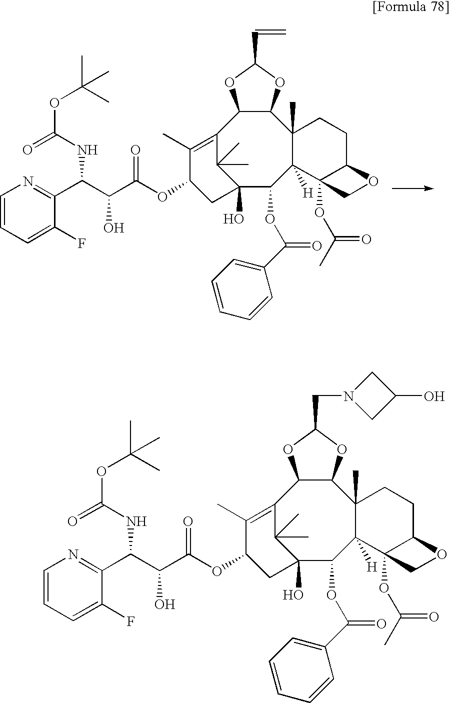

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

(2R,3S)-3-(tert-butoxycarbonylamino)-3-(3-fluoro-2-pyridyl)-2-hydroxypropionate and 3-azetidinol hydrochloride were used as starting materials to perform the same procedure as that of Example 1 and thereby to obtain the title compound as white amorphous solid.

¹H-NMR (400 MHz, CDCl₃) δ: 1.24-2.16 (8H, m), 1.29 (3H, s), 1.40 (9H, s), 1.49 (3H, s), 1.61 (3H, s), 1.78 (3H, s), 2.35 (3H, s), 2.42-2.48 (1H, m), 2.79 (1H, dd, J=12.3, 5.6 Hz), 2.88 (1H, dd, J=12.3, 4.2 Hz), 2.93 (1H, d, J=5.1 Hz), 3.11 (2H, dd, J=13.3, 7.2 Hz), 3.74-3.80 (2H, m), 4.14 (1H, d, J=7.3 Hz), 4.23 (1H, d, J=8.3 Hz), 4.32 (1H, d, J=8.3 Hz), 4.47 (1H, t, J=5.7 Hz), 4.68 (1H, d, J=2.4 Hz), 4.88 (1H, t, J=4.9 Hz), 4.91 (1H, s), 5.22 (1H, d, J=7.1 Hz), 5.64 (1H, d, J=8.5 Hz), 5.98 (1H, d, J=4.9 Hz), 6.08 (1H, t, J=8.5 Hz), 6.24 (1H, d, J=8.5 Hz), 7.28-7.33 (1H, m), 7.43-7.49 (3H, m), 7.60 (1H, t, J=7.6 Hz), 8.13 (2H, d, J=7.6 Hz), 8.40 (1H, d, J=4.6 Hz).

FAB-MS; m/z: 910 (M+H)⁺.

### Example 7

### (1S,2S,3R,4S,5R,8R,9S,10R,13S)-4-(Acetoxy)-2-(benzoyloxy)-5,20-epoxy-1-hydroxy-9,10-[(1S)-2-(3-methoxyazetidino)ethylidenedioxy]tax-11-en-13-yl

### (2R,3S)-3-(tert-butoxycarbonylamino)-2-hydroxy-3-(2-pyridyl)propionate

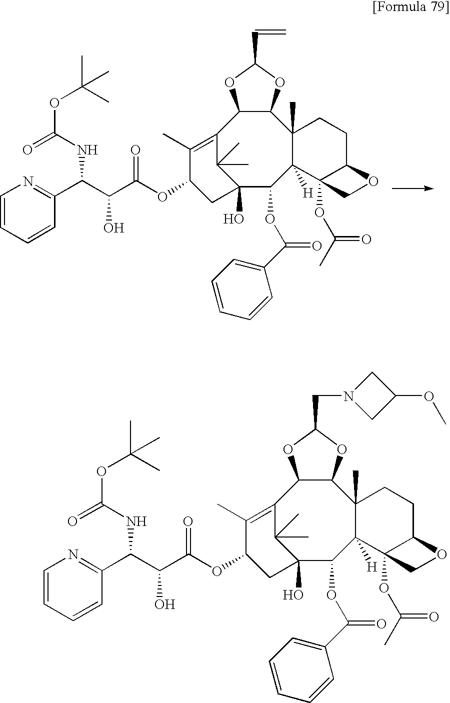

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-2-hydroxy-3-(2-pyridyl)propionate and the compound obtained in Reference Example 1 were used as starting materials to perform the same procedure as that of Example 1 and thereby to obtain the title compound as white amorphous solid.

¹H-NMR (400 MHz, CDCl₃) δ 1.18-2.13 (7H, m), 1.25 (3H, s), 1.44 (9H, s), 1.48 (3H, s), 1.60 (3H, s), 1.71 (3H, s), 2.31-2.37 (1H, m), 2.35 (3H, s), 2.80 (1H, dd, J=12.5, 5.5 Hz), 2.88 (1H, dd, J=12.5, 4.4 Hz), 2.92 (1H, d, J=5.1 Hz), 3.06-3.12 (2H, m), 3.27 (3H, s), 3.72-3.77 (2H, m), 4.07 (1H, t, J=5.9 Hz), 4.12 (1H, d, J=7.3 Hz), 4.21 (1H, d, J=8.3 Hz), 4.32 (1H, d, J=8.3 Hz), 4.86-4.91 (3H, m), 5.20 (1H, d, J=7.1 Hz), 5.35 (1H, d, J=9.8 Hz), 5.95-5.98 (2H, m), 6.08 (1H, t, J=8.3 Hz), 7.21-7.24 (1H, m), 7.41-7.49 (3H, m), 7.60 (1H, t, J=7.6 Hz), 7.72 (1H, dt, J=7.6, 1.5 Hz), 8.13 (2H, d, J=7.6 Hz), 8.53 (1H, d, J=4.4 Hz).

FAB-MS; m/z: 906 (M+H)⁺.

### Example 8

### (1S,2S,3R,4S,5R,8R,9S,10R,13S)-4-(Acetoxy)-2-(benzoyloxy)-5,20-epoxy-1-hydroxy-9,10-[(1S)-2-(3-methoxyazetidino)ethylidenedioxy]tax-11-en-13-yl

### (2R,3S)-3-(tert-butoxycarbonylamino)-3-(3-fluoro-2-pyridyl)-2-hydroxypropionate

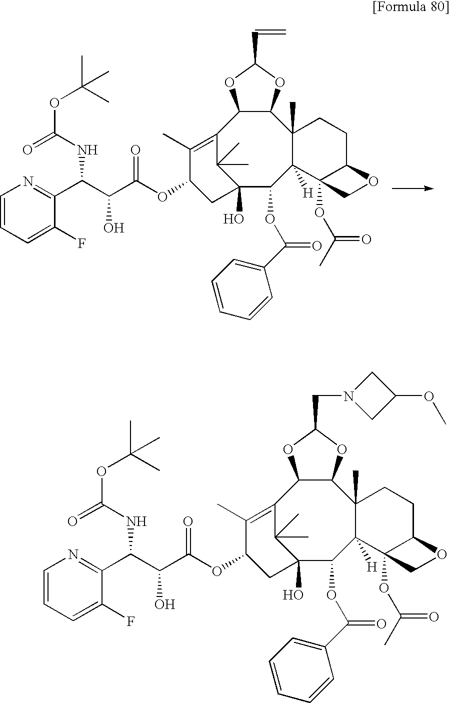

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

(2R,3S)-3-(tert-butoxycarbonylamino)-3-(3-fluoro-2-pyridyl)-2-hydroxypropionate and the compound obtained in Reference Example 1 were used as starting materials to perform the same procedure as that of Example 1 and thereby to obtain the title compound as white amorphous solid.

¹H-NMR (400 MHz, CDCl₃) δ: 1.26-2.15 (7H, m), 1.28 (3H, s), 1.40 (9H, s), 1.49 (3H, s), 1.61 (3H, s), 1.78 (3H, s), 2.34 (3H, s), 2.42-2.48 (1H, m), 2.80 (1H, dd, J=12.5, 5.6 Hz), 2.89 (1H, dd, J=12.5, 4.4 Hz), 2.93 (1H, d, J=5.1 Hz), 3.06-3.12 (2H, m), 3.28 (3H, s), 3.73-3.77 (2H, m), 4.08 (1H, t, J=5.7 Hz), 4.14 (1H, d, J=7.3 Hz), 4.23 (1H, d, J=8.3 Hz), 4.33 (1H, d, J=8.3 Hz), 4.67 (1H, d, J=2.4 Hz), 4.87-4.91 (2H, m), 5.22 (1H, d, J=7.1 Hz), 5.65 (1H, d, J=8.5 Hz), 5.99 (1H, d, J=5.1 Hz), 6.08 (1H, t, J=8.5 Hz), 6.22 (1H, d, J=8.5 Hz), 7.33-7.33 (1H, m), 7.43-7.49 (3H, m), 7.60 (1H, t, J=7.6 Hz), 8.14 (2H, d, J=7.6 Hz), 8.40 (1H, d, J=4.6 Hz).

FAB-MS; m/z: 924 (M+H)⁺.

### Example 9

### (1S,2S,3R,4S,5R,8R,9S,10R,13S)-4-(Acetoxy)-2-(benzoyloxy)-9,10-{(1S)-[2-(3-dimethylcarbamoyl)azetidino]ethylidenedioxy}-5,20-epoxy-1-hydroxytax-11-en-13-yl

### (2R,3S)-3-(tert-butoxycarbonylamino)-2-hydroxy-3-(2-pyridyl)propionate

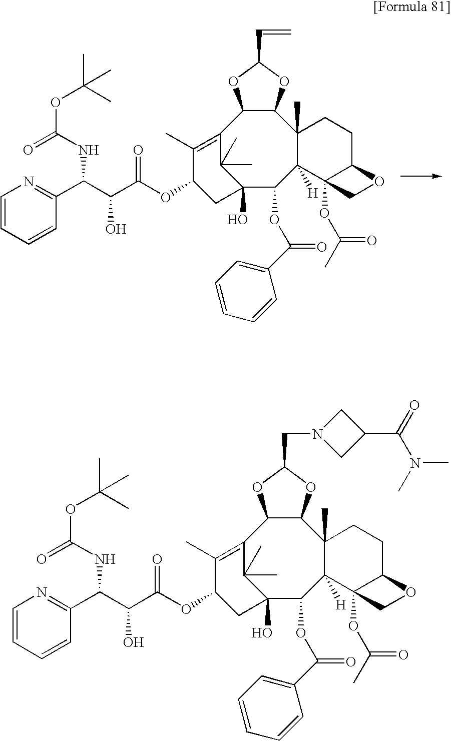

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-2-hydroxy-3-(2-pyridyl)propionate and the compound obtained in Reference Example 2, Step 2 were used as starting materials to perform the same procedure as that of Example 1 and thereby to obtain the title compound as white amorphous solid.

¹H-NMR (400 MHz, CDCl₃) δ: 1.22-2.12 (6H, m), 1.25 (3H, s), 1.44 (9H, s), 1.48 (3H, s), 1.60 (3H, s), 1.71 (3H, s), 2.31-2.37 (1H, m), 2.35 (3H, s), 2.76-2.92 (4H, m), 2.91 (3H, s), 2.95 (3H, s), 3.39-3.58 (3H, m), 3.71 (2H, dd, J=13.7, 6.6 Hz), 4.12 (1H, d, J=7.3 Hz), 4.21 (1H, d, J=8.3 Hz), 4.32 (1H, d, J=8.3 Hz), 4.85-4.91 (3H, m), 5.20 (1H, d, J=6.6 Hz), 5.35 (1H, d, J=9.0 Hz), 5.95-5.97 (2H, m), 6.08 (1H, t, J=8.5 Hz), 7.21-7.25 (1H, m), 7.41-7.49 (3H, m), 7.60 (1H, t, J=7.3 Hz), 7.72 (1H, t, J=7.3 Hz), 8.13 (2H, d, J=7.3 Hz), 8.54 (1H, d, J=4.2 Hz).

FAB-MS; m/z: 947 (M+H)⁺.

### Example 10

### (1S,2S,3R,4S,5R,8R,9S,10R,13S)-4-(Acetoxy)-2-(benzoyloxy)-9,10-{(1S)-[2-(3-dimethylcarbamoyl)azetidino]ethylidenedioxy}-5,20-epoxy-1-hydroxytax-11-en-13-yl

### (2R,3S)-3-(tert-butoxycarbonylamino)-3-(3-fluoro-2-pyridyl)-2-hydroxypropionate

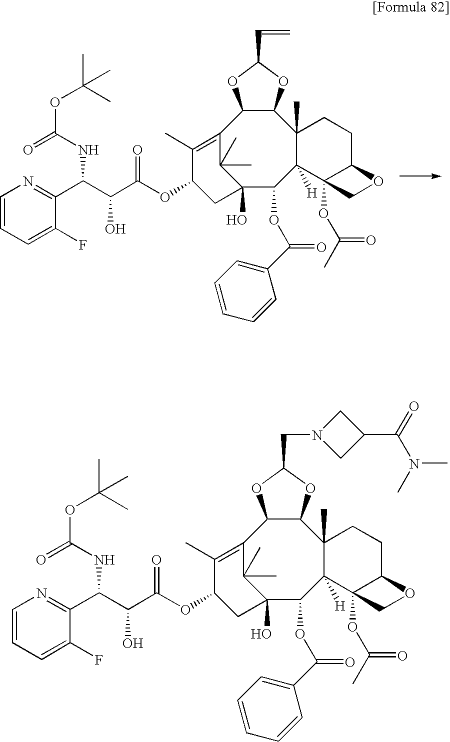

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-3-(3-fluoro-2-pyridyl)-2-hydroxypropionate and the compound obtained in Reference Example 2, Step 2 were used as starting materials to perform the same procedure as that of Example 1 and thereby to obtain the title compound as white amorphous solid.

¹H-NMR (400 MHz, CDCl₃) δ: 1.24-2.14 (7H, m), 1.28 (3H, s), 1.40 (9H, s), 1.49 (3H, s), 1.61 (3H, s), 1.78 (3H, s), 2.34 (3H, s), 2.45 (1H, dd, J=14.8, 9.6 Hz), 2.79 (1H, dd, J=12.6, 5.9 Hz), 2.86 (1H, dd, J=12.6, 4.5 Hz), 2.91 (3H, s), 2.93 (1H, d, J=5.4 Hz), 2.96 (3H, s), 3.39-3.47 (2H, m), 3.50-3.58 (1H, m), 3.69-3.74 (2H, m), 4.13 (1H, d, J=7.1 Hz), 4.23 (1H, d, J=8.3 Hz), 4.32 (1H, d, J=8.3 Hz), 4.67 (1H, d, J=2.4 Hz), 4.87 (1H, t, J=4.9 Hz), 4.91 (1H, brs), 5.21 (1H, d, J=7.1 Hz), 5.65 (1H, d, J=8.5 Hz), 5.98 (1H, d, J=5.1 Hz), 6.08 (1H, t, J=8.5 Hz), 6.21 (1H, d, J=8.3 Hz), 7.28-7.32 (1H, m), 7.42-7.49 (3H, m), 7.60 (1H, t, J=7.4 Hz), 8.14 (2H, d, J=7.4 Hz), 8.40 (1H, d, J=4.6 Hz).

FAB-MS; m/z: 965 (M+H)⁺.

### Example 11

### (1S,2S,3R,4S,5R,8R,9S,10R,13S)-4-(Acetoxy)-2-(benzoyloxy)-5,20-epoxy-1-hydroxy-9,10-[(1S)-2-(3-hydroxy-3-methylazetidino)ethylidenedioxy]tax-11-en-13-yl

### (2R,3S)-3-(tert-butoxycarbonylamino)-2-hydroxy-3-(2-pyridyl)propionate

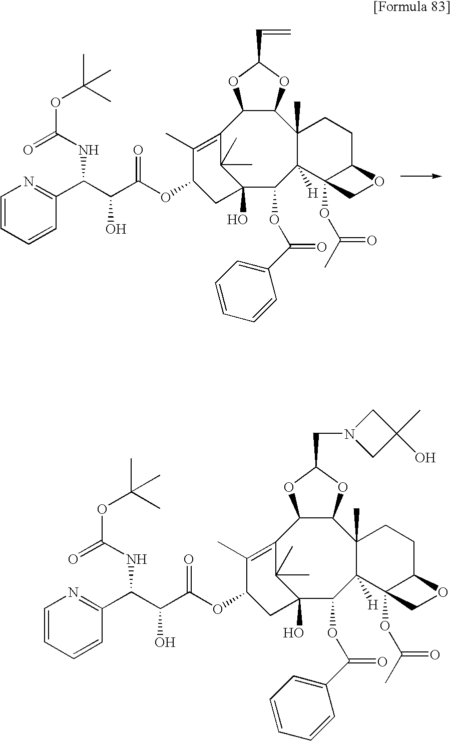

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-2-hydroxy-3-(2-pyridyl)propionate and the compound obtained in Reference Example 3 were used as starting materials to perform the same procedure as that of Example 1 and thereby to obtain the title compound as white amorphous solid.

¹H-NMR (400 MHz, CDCl₃) δ: 1.21-2.12 (8H, m), 1.26 (3H, s), 1.43 (9H, s), 1.48 (3H, s), 1.51 (3H, s), 1.60 (3H, s), 1.71 (3H, s), 2.31-2.37 (1H, m), 2.35 (3H, s), 2.78 (1H, dd, J=12.5, 5.6 Hz), 2.88-2.92 (2H, m), 3.21 (2H, t, J=8.1 Hz), 3.42 (2H, t, J=8.1 Hz), 4.12 (1H, d, J=7.1 Hz), 4.21 (1H, d, J=8.5 Hz), 4.32 (1H, d, J=8.5 Hz), 4.88-4.92 (3H, m), 5.20 (1H, d, J=7.1 Hz), 5.35 (1H, d, J=8.5 Hz), 5.97 (2H, d, J=5.9 Hz), 6.08 (1H, t, J=8.3 Hz), 7.21-7.24 (1H, m), 7.41-7.49 (3H, m), 7.60 (1H, t, J=7.2 Hz), 7.71 (1H, t, J=7.8 Hz), 8.12 (2H, d, J=7.8 Hz), 8.53 (1H, d, J=4.4 Hz).

FAB-MS; m/z: 906 (M+H)⁺.

### Example 12

### (1S,2S,3R,4S,5R,8R,9S,10R,13S)-4-(Acetoxy)-2-(benzoyloxy)-5,20-epoxy-1-hydroxy-9,10-[(1S)-2-(3-hydroxy-3-methylazetidino)ethylidenedioxy]tax-11-en-13-yl

### (2R,3S)-3-(tert-butoxycarbonylamino)-3-(3-fluoro-2-pyridyl)-2-hydroxypropionate

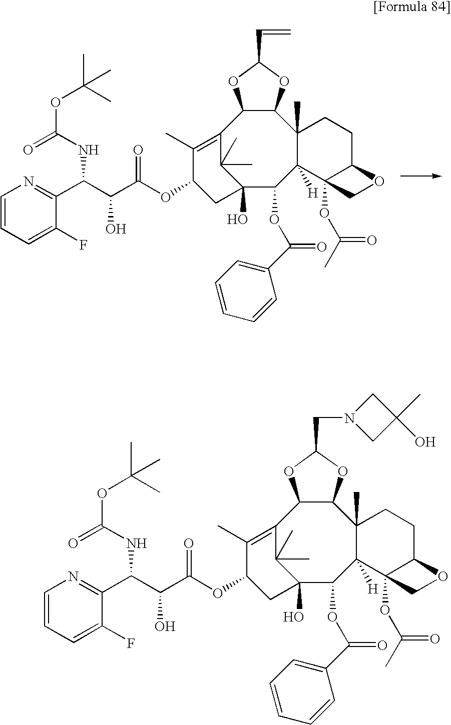

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-3-(3-fluoro-2-pyridyl)-2-hydroxypropionate and the compound obtained in Reference Example 3 were used as starting materials to perform the same procedure as that of Example 1 and thereby to obtain the title compound as white amorphous solid.

¹H-NMR (400 MHz, CDCl₃) δ: 1.24-2.13 (8H, m), 1.29 (3H, s), 1.40 (9H, s), 1.50 (3H, s), 1.52 (3H, s), 1.61 (3H, s), 1.78 (3H, s), 2.34 (3H, s), 2.45 (1H, dd, J=14.9, 9.3 Hz), 2.80 (1H, dd, J=12.5, 5.9 Hz), 2.88-2.94 (2H, m), 3.21 (2H, t, J=8.2 Hz), 3.44 (2H, t, J=7.8 Hz), 4.14 (1H, d, J=7.1 Hz), 4.23 (1H, d, J=8.5 Hz), 4.32 (1H, d, J=8.5 Hz), 4.67 (1H, d, J=2.7 Hz), 4.89-4.92 (2H, m), 5.22 (1H, d, J=6.8 Hz), 5.65 (1H, d, J=8.3 Hz), 5.99 (1H, d, J=4.9 Hz), 6.09 (1H, t, J=8.4 Hz), 6.21 (1H, d, J=8.1 Hz), 7.28-7.32 (1H, m), 7.42-7.49 (3H, m), 7.60 (1H, t, J=7.4 Hz), 8.12-8.15 (2H, m), 8.39-8.41 (1H, m).

FAB-MS; m/z: 924 (M+H)⁺.

### Example 13

### (1S,2S,3R,4S,5R,8R,9S,10R,13S)-4-(Acetoxy)-2-(benzoyloxy)-5,20-epoxy-9,10-[(1S)-2-(3-ethyl-3-hydroxyazetidino)ethylidenedioxy]-1-hydroxytax-11-en-13-yl

### (2R,3S)-3-(tert-butoxycarbonylamino)-3-(3-fluoro-2-pyridyl)-2-hydroxypropionate

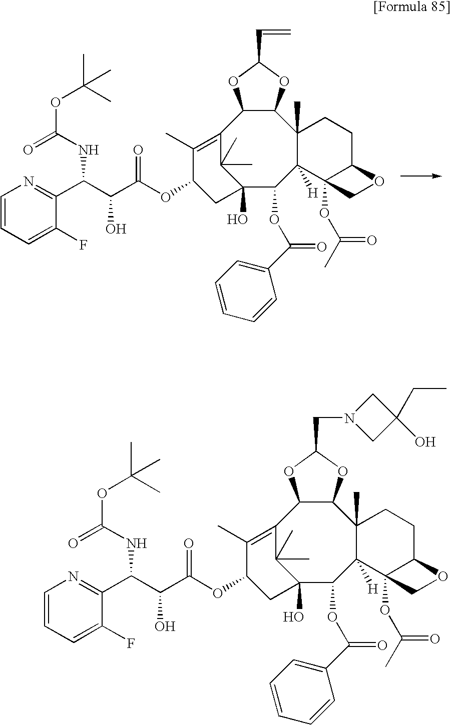

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-3-(3-fluoro-2-pyridyl)-2-hydroxypropionate and the compound obtained in Reference Example 4, Step 2 were used as starting materials to perform the same procedure as that of Example 1 and thereby to obtain the title compound as white amorphous solid.

¹H-NMR (400 MHz, CDCl₃) δ: 0.97 (3H, t, J=7.3 Hz), 1.21-2.48 (11H, m), 1.29 (3H, s), 1.40 (9H, s), 1.50 (3H, s), 1.62 (3H, s), 1.78 (3H, s), 2.34 (3H, s), 2.82 (1H, dd, J=12.5, 5.9 Hz), 2.88-2.95 (2H, m), 3.12-3.22 (2H, m), 3.46 (2H, t, J=6.8 Hz), 4.14 (1H, d, J=7.1 Hz), 4.23 (1H, d, J=8.5 Hz), 4.33 (1H, d, J=8.5 Hz), 4.68 (1H, d, J=2.4 Hz), 4.88-4.92 (2H, m), 5.22 (1H, d, J=6.3 Hz), 5.65 (1H, d, J=7.6 Hz), 5.99 (1H, d, J=5.1 Hz), 6.09 (1H, t, J=8.8 Hz), 6.17-6.23 (1H, m), 7.28-7.33 (1H, m), 7.41-7.50 (3H, m), 7.57-7.62 (1H, m), 8.10-8.15 (2H, m), 8.38-8.41 (1H, m).

FAB-MS; m/z: 938 (M+H)⁺.

### Example 14

### (1S,2S,3R,4S,5R,8R,9S,10R,13S)-4-(Acetoxy)-2-(benzoyloxy)-9,10-[(1S)-2-(3-cyclopropyl-3-hydroxyazetidino)ethylidenedioxy]-5,20-epoxy-1-hydroxytax-11-en-13-yl

### (2R,3S)-3-(tert-butoxycarbonylamino)-3-(3-fluoro-2-pyridyl)-2-hydroxypropionate

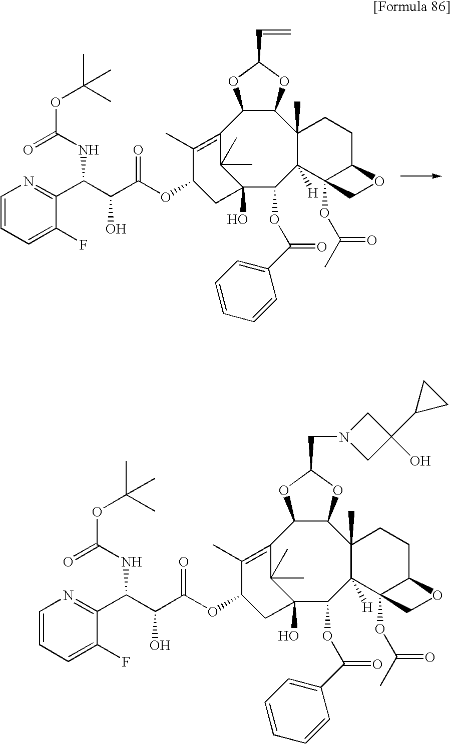

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-3-(3-fluoro-2-pyridyl)-2-hydroxypropionate and the compound obtained in Reference Example 5, Step 2 were used as starting materials to perform the same procedure as that of Example 1 and thereby to obtain the title compound as white amorphous solid.

¹H-NMR (400 MHz, CDCl₃) δ: 0.43-0.48 (2H, m), 0.51-0.58 (2H, m), 1.16-2.50 (10H, m), 1.29 (3H, s), 1.41 (9H, s), 1.50 (3H, s), 1.61 (3H, s), 1.78 (3H, s), 2.34 (3H, s), 2.79 (1H, dd, J=12.5, 5.9 Hz), 2.86-2.95 (2H, m), 3.17-3.24 (2H, m), 3.33-3.39 (2H, m), 4.13 (1H, d, J=7.1 Hz), 4.23 (1H, d, J=8.3 Hz), 4.33 (1H, d, J=8.3 Hz), 4.68 (1H, d, J=2.7 Hz), 4.87-4.93 (2H, m), 5.21 (1H, d, J=6.8 Hz), 5.65 (1H, d, J=8.3 Hz), 5.99 (1H, d, J=5.1 Hz), 6.09 (1H, t, J=8.7 Hz), 6.21 (1H, d, J=9.0 Hz), 7.28-7.33 (1H, m), 7.42-7.50 (3H, m), 7.60 (1H, t, J=7.4 Hz), 8.14 (2H, d, J=7.3 Hz), 8.40 (1H, d, J=4.6 Hz).

FAB-MS; m/z: 950 (M+H)⁺.

### Example 15

### (1S,2S,3R,4S,5R,8R,9S,10R,13S)-4-(Acetoxy)-2-(benzoyloxy)-5,20-epoxy-1-hydroxy-9,10-[(1S)-2-(3-hydroxy-3-trifluoromethylazetidino)ethylidenedioxy]tax-11-en-13-yl

### (2R,3S)-3-(tert-butoxycarbonylamino)-3-(3-fluoro-2-pyridyl)-2-hydroxypropionate

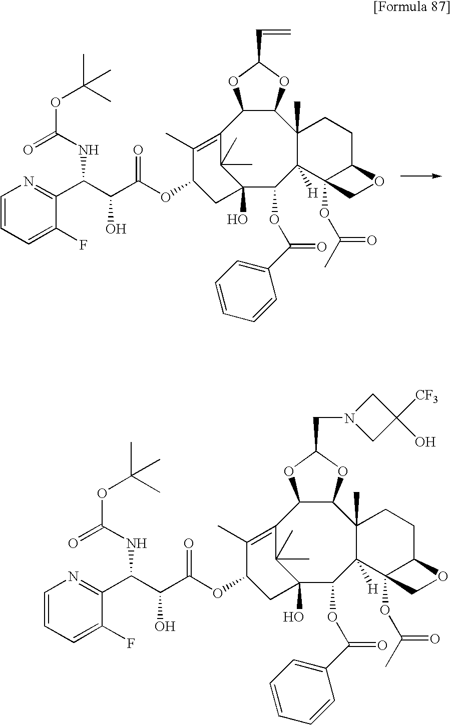

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-3-(3-fluoro-2-pyridyl)-2-hydroxypropionate and the compound obtained in Reference Example 6, Step 2 were used as starting materials to perform the same procedure as that of Example 1 and thereby to obtain the title compound as white amorphous solid.

¹H-NMR (400 MHz, CDCl₃) δ: 0.96-2.53 (7H, m), 1.29 (3H, s), 1.40 (9H, s), 1.50 (3H, s), 1.61 (3H, s), 1.78 (3H, s), 2.35 (3H, s), 2.86 (1H, dd, J=12.5, 5.6 Hz), 2.92-2.97 (2H, m), 3.34-3.54 (4H, m), 3.69-3.78 (2H, m), 4.09-4.16 (1H, m), 4.23 (1H, d, J=8.3 Hz), 4.33 (1H, d, J=8.3 Hz), 4.68 (1H, d, J=2.7 Hz), 4.89-4.93 (2H, m), 5.21 (1H, d, J=6.8 Hz), 5.64 (1H, d, J=9.0 Hz), 5.98 (1H, d, J=4.9 Hz), 6.09 (1H, t, J=8.5 Hz), 6.25 (1H, d, J=8.5 Hz), 7.28-7.33 (1H, m), 7.43-7.50 (3H, m), 7.61 (1H, t, J=7.4 Hz), 8.14 (2H, d, J=7.3 Hz), 8.40 (1H, d, J=4.6 Hz).

FAB-MS; m/z: 978 (M+H)⁺.

### Example 16

### (1S,2S,3R,4S,5R,8R,9S,10R,13S)-4-(Acetoxy)-2-(benzoyloxy)-9,10-[(1S)-2-(3-cyanoazetidino)ethylidenedioxy]-5,20-epoxy-1-hydroxytax-11-en-13-yl

### (2R,3S)-3-(tert-butoxycarbonylamino)-2-hydroxy-3-(2-pyridyl)propionate

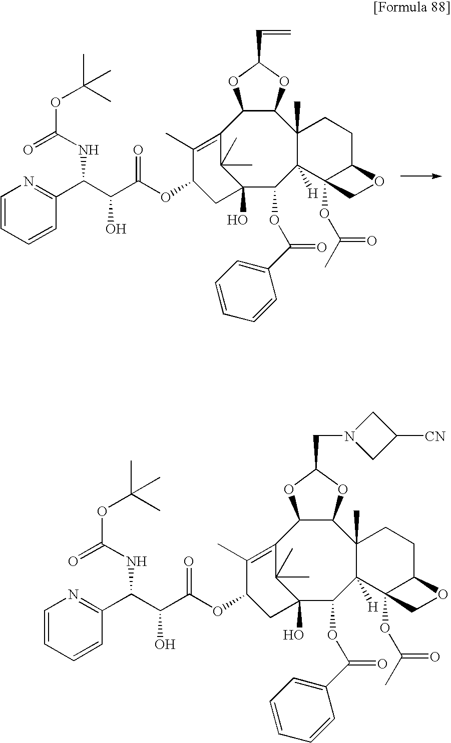

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-2-hydroxy-3-(2-pyridyl)propionate and the compound obtained in Reference Example 7 were used as starting materials to perform the same procedure as that of Example 1 and thereby to obtain the title compound as white amorphous solid.

¹H-NMR (400 MHz, CDCl₃) δ: 1.24-2.14 (7H, m), 1.26 (3H, s), 1.44 (9H, s), 1.48 (3H, s), 1.58 (3H, s), 1.71 (3H, s), 2.31-2.37 (1H, m), 2.35 (3H, s), 2.77 (1H, dd, J=12.5, 5.6 Hz), 2.85 (1H, dd, J=12.5, 4.6 Hz), 2.92 (1H, d, J=5.1 Hz), 3.30-3.35 (1H, m), 3.45-3.55 (2H, m), 3.70-3.75 (2H, m), 4.12 (1H, dd, J=7.3, 1.7 Hz), 4.21 (1H, d, J=8.5 Hz), 4.33 (1H, d, J=8.5 Hz), 4.83-4.92 (3H, m), 5.20 (1H, d, J=7.1 Hz), 5.35 (1H, d, J=7.8 Hz), 5.92-5.98 (2H, m), 6.09 (1H, t, J=7.6 Hz), 7.22-7.26 (1H, m), 7.41-7.49 (3H, m), 7.60 (1H, t, J=7.4 Hz), 7.72 (1H, t, J=7.4 Hz), 8.13 (2H, d, J=7.8 Hz), 8.53 (1H, d, J=4.6 Hz).

FAB-MS; m/z: 901 (M+H)⁺, 923 (M+Na)⁺.

### Example 17

### (1S,2S,3R,4S,5R,8R,9S,10R,13S)-4-(Acetoxy)-2-(benzoyloxy)-5,20-epoxy-1-hydroxy-9,10-[(1S)-2-(3-hydroxymethylazetidino)ethylidenedioxy]tax-11-en-13-yl

### (2R,3S)-3-(tert-butoxycarbonylamino)-2-hydroxy-3-(2-pyridyl)propionate

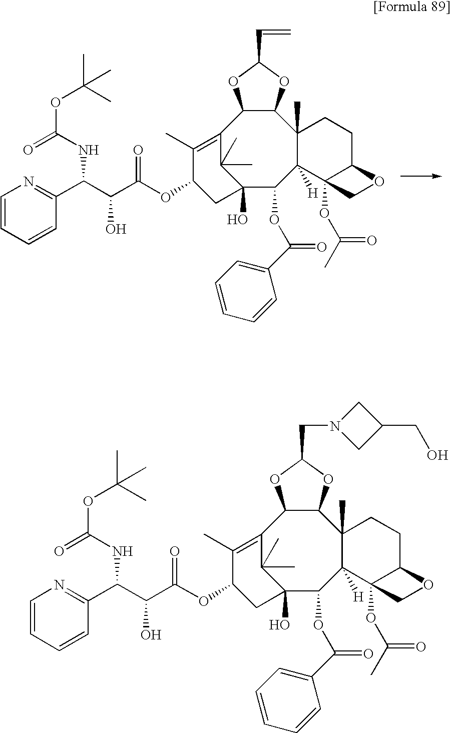

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-2-hydroxy-3-(2-pyridyl)propionate and the compound obtained in Reference Example 8 were used as starting materials to perform the same procedure as that of Example 1 and thereby to obtain the title compound as white amorphous solid.

¹H-NMR (400 MHz, CDCl₃) δ: 1.23-2.12 (8H, m), 1.25 (3H, s), 1.44 (9H, s), 1.49 (3H, s), 1.60 (3H, s), 1.70 (3H, s), 2.31-2.37 (1H, m), 2.34 (3H, s), 2.65-2.71 (1H, m), 2.76 (1H, dd, J=12.5, 5.9 Hz), 2.85 (1H, dd, J=12.5, 4.2 Hz), 2.92 (1H, d, J=5.1 Hz), 3.21-3.26 (2H, m), 3.47-3.53 (2H, m), 3.80 (2H, d, J=5.6 Hz), 4.11 (1H, d, J=7.1 Hz), 4.21 (1H, d, J=8.3 Hz), 4.33 (1H, d, J=8.3 Hz), 4.86-4.92 (3H, m), 5.19 (1H, d, J=6.8 Hz), 5.35 (1H, d, J=9.3 Hz), 5.94-5.99 (2H, m), 6.08 (1H, t, J=7.9 Hz), 7.21-7.25 (1H, m), 7.41-7.49 (3H, m), 7.60 (1H, t, J=7.3 Hz), 7.72 (1H, dt, J=7.6, 1.7 Hz), 8.13 (2H, d, J=7.6 Hz), 8.54 (1H, d, J=4.6 Hz).

FAB-MS; m/z: 906 (M+H)⁺.

### Example 18

### (1S,2S,3R,4S,5R,8R,9S,10R,13S)-4-(Acetoxy)-9,10-{(1S)-2-[3-(N-acetyl-N-methylamino)azetidino]ethylidenedioxy}-2-(benzoyloxy)-5,20-epoxy-1-hydroxytax-11-en-13-yl

### (2R,3S)-3-(tert-butoxycarbonylamino)-3-(3-fluoro-2-pyridyl)-2-hydroxypropionate

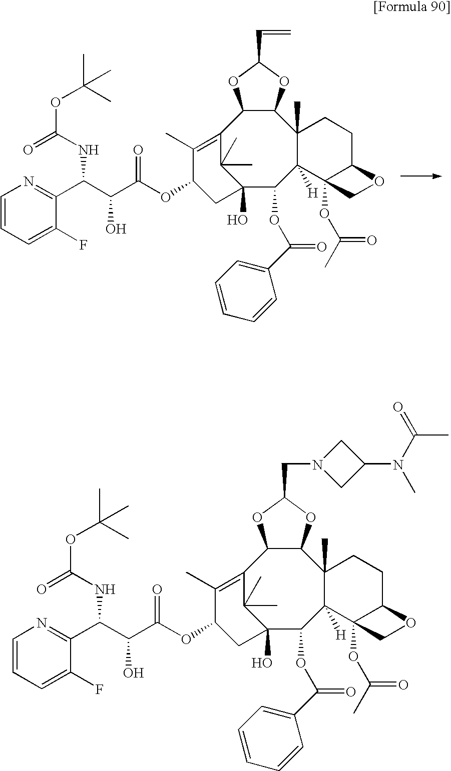

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-3-(3-fluoro-2-pyridyl)-2-hydroxypropionate and the compound obtained in Reference Example 9, Step 2 were used as starting materials to perform the same procedure as that of Example 1 and thereby to obtain the title compound as white amorphous solid.

¹H-NMR (400 MHz, CDCl₃) δ: 1.26-2.13 (7H, m), 1.29 (3H, s), 1.40 (9H, s), 1.50 (3H, s), 1.61 (3H, s), 1.78 (3H, s), 2.08, 2.10 (total 3H, each s), 2.35 (3H, s), 2.42-2.49 (1H, m), 2.76-2.99 (3H, m), 3.03, 3.08 (total 3H, each s), 3.22-3.36 (2H, m), 3.73 (2H, dd, J=13.9, 7.1 Hz), 4.13-4.18 (1H, m), 4.23 (1H, d, J=8.3 Hz), 4.33 (1H, d, J=8.3 Hz), 4.55-4.98 (3H, m), 4.68 (1H, d, J=2.7 Hz), 5.21-5.28 (1H, m), 5.64 (1H, d, J=8.8 Hz), 5.99 (1H, d, J=5.1 Hz), 6.09 (1H, t, J=8.3 Hz), 6.21 (1H, d, J=8.3 Hz), 7.28-7.33 (1H, m), 7.43-7.49 (3H, m), 7.60 (1H, t, J=7.4 Hz), 8.13 (2H, d, J=7.4 Hz), 8.40 (1H, d, J=4.4 Hz).

FAB-MS; m/z: 965 (M+H)⁺.

### Example 19

### (1S,2S,3R,4S,5R,8R,9S,10R,13S)-4-(Acetoxy)-2-(benzoyloxy)-5,20-epoxy-1-hydroxy-9,10-{(1S)-2-[3-(N-methanesulfonyl-N-methylamino)azetidino]ethylidenedioxy}tax-11-en-13-yl (2R,3S)-3-(tert-butoxycarbonylamino)-3-(3-fluoro-2-pyridyl)-2-hydroxypropionate

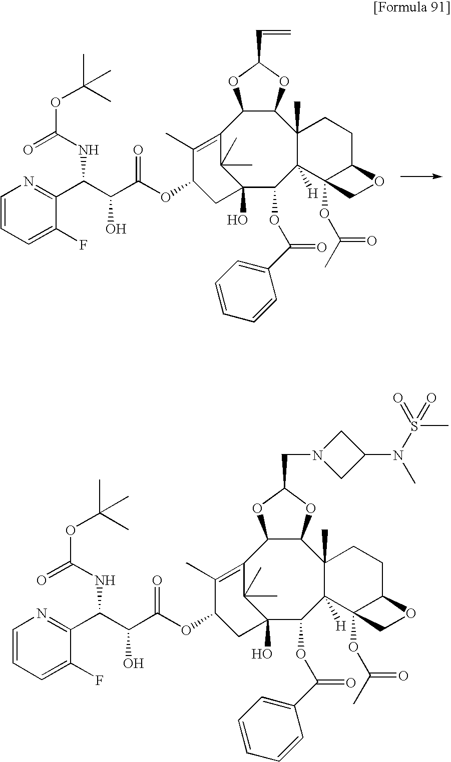

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-3-(3-fluoro-2-pyridyl)-2-hydroxypropionate and the compound obtained in Reference Example 10, Step 2 were used as starting materials to perform the same procedure as that of Example 1 and thereby to obtain the title compound as white amorphous solid.

¹H-NMR (400 MHz, CDCl₃) δ: 1.26-2.16 (7H, m), 1.29 (3H, s), 1.40 (9H, s), 1.50 (3H, s), 1.61 (3H, s), 1.78 (3H, s), 2.34 (3H, s), 2.42-2.48 (1H, m), 2.76-2.94 (3H, m), 2.78 (3H, s), 2.91 (3H, s), 3.32 (2H, dd, J=17.6, 7.6 Hz), 3.70 (2H, dd, J=13.1, 6.0 Hz), 4.14 (1H, d, J=7.3 Hz), 4.23 (1H, d, J=8.5 Hz), 4.27-4.34 (2H, m), 4.67 (1H, d, J=2.0 Hz), 4.86 (1H, t, J=5.0 Hz), 4.91 (1H, s), 5.22 (1H, d, J=6.8 Hz), 5.64 (1H, d, J=8.1 Hz), 5.99 (1H, d, J=4.9 Hz), 6.09 (1H, t, J=8.3 Hz), 6.20 (1H, d, J=8.3 Hz), 7.28-7.33 (1H, m), 7.43-7.50 (3H, m), 7.60 (1H, t, J=7.4 Hz), 8.12-8.14 (2H, m), 8.40 (1H, d, J=4.6 Hz).

FAB-MS; m/z: 1001 (M+H)⁺.

### Example 20

### (1S,2S,3R,4S,5R,8R,9S,10R,13S)-4-(Acetoxy)-2-(benzoyloxy)-5,20-epoxy-1-hydroxy-9,10-{(1S)-2-[(2S)-2-methoxymethylazetidino]ethylidenedioxy}tax-11-en-13-yl

### (2R,3S)-3-(tert-butoxycarbonylamino)-3-(3-fluoro-2-pyridyl)-2-hydroxypropionate

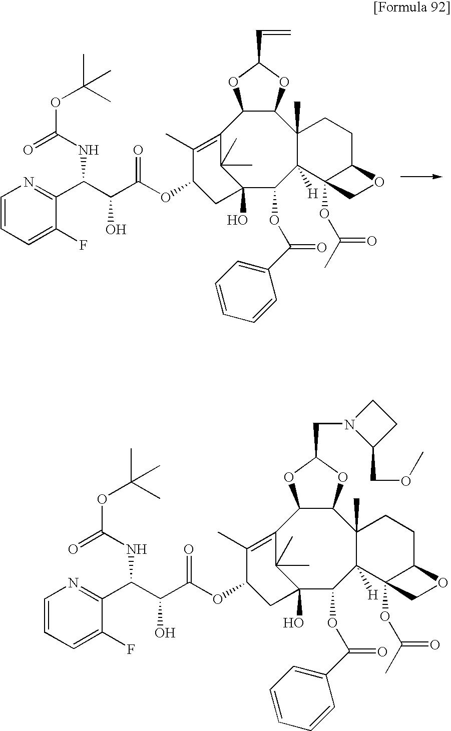

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-3-(3-fluoro-2-pyridyl)-2-hydroxypropionate and the compound obtained in Reference Example 11, Step 2 were used as starting materials to perform the same procedure as that of Example 1 and thereby to obtain the title compound as white amorphous solid.

¹H-NMR (400 MHz, CDCl₃) δ: 1.24-2.10 (8H, m), 1.28 (3H, s), 1.40 (9H, s), 1.49 (3H, s), 1.61 (3H, s), 1.78 (3H, s), 2.34 (3H, s), 2.42-2.48 (1H, m), 2.77 (1H, dd, J=12.8, 4.8 Hz), 2.93-3.04 (3H, m), 3.38 (3H, s), 3.43-3.55 (4H, m), 4.11-4.14 (2H, m), 4.23 (1H, d, J=8.3 Hz), 4.33 (1H, d, J=8.3 Hz), 4.67 (1H, d, J=2.2 Hz), 4.88-4.92 (2H, m), 5.22 (1H, d, J=7.6 Hz), 5.64 (1H, d, J=8.3 Hz), 5.99 (1H, d, J=5.1 Hz), 6.08 (1H, t, J=8.5 Hz), 6.20 (1H, d, J=8.5 Hz), 7.28-7.32 (1H, m), 7.42-7.49 (3H, m), 7.60 (1H, t, J=7.4 Hz), 8.12-8.15 (2H, m), 8.40 (1H, d, J=4.6 Hz).

FAB-MS; m/z: 938 (M+H)⁺.

### Example 21

### (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-11-en-13-yl

### (2R,3S)-3-(tert-butoxycarbonylamino)-2-hydroxy-3-(2-pyridyl)propionate

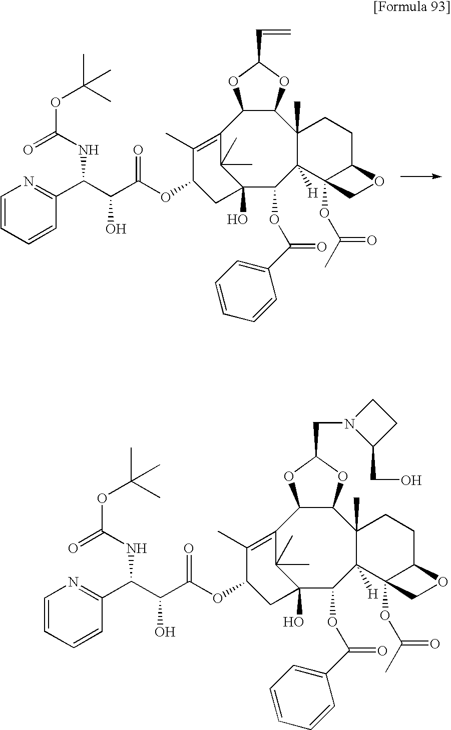

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-2-hydroxy-3-(2-pyridyl)propionate and the compound obtained in Reference Example 25 were used as starting materials to perform the same procedure as that of Example 1 and thereby to obtain the title compound as white amorphous solid.

¹H-NMR (400 MHz, CDCl₃) δ: 1.23-2.12 (9H, m), 1.25 (3H, s), 1.44 (9H, s), 1.49 (3H, s), 1.58 (3H, s), 1.70 (3H, s), 2.20-2.37 (2H, m), 2.35 (3H, s), 2.83 (1H, dd, J=12.9, 4.4 Hz), 2.88-2.93 (2H, m), 3.11 (1H, dd, J=15.7, 8.7 Hz), 3.41-3.50 (3H, m), 3.64-3.67 (1H, m), 4.13 (1H, d, J=7.6 Hz), 4.21 (1H, d, J=8.5 Hz), 4.33 (1H, d, J=8.5 Hz), 4.86-4.92 (3H, m), 5.22 (1H, d, J=7.1 Hz), 5.35 (1H, d, J=9.3 Hz), 5.93-5.97 (2H, m), 6.08 (1H, t, J=8.1 Hz), 7.22-7.25 (1H, m), 7.41-7.49 (3H, m), 7.60 (1H, t, J=7.6 Hz), 7.72 (1H, dt, J=7.6, 1.7 Hz), 8.11-8.14 (2H, m), 8.54 (1H, d, J=4.6 Hz).

FAB-MS; m/z: 906 (M+H)⁺.

### Example 22

### (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-11-en-13-yl

### (2R,3S)-3-(tert-butoxycarbonylamino)-3-(3-fluoro-2-pyridyl)-2-hydroxypropionate

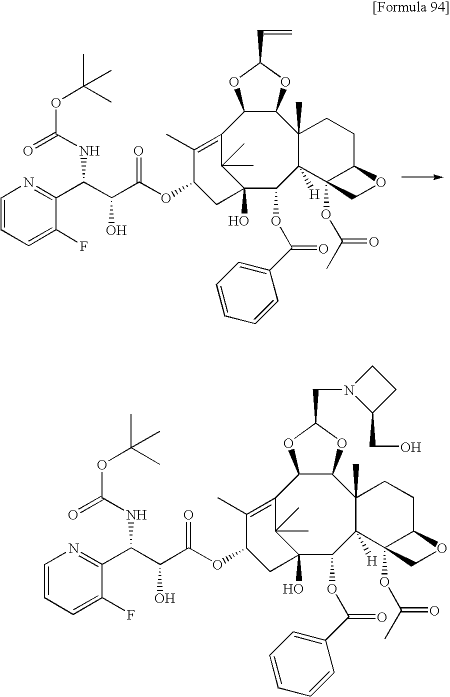

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-3-(3-fluoro-2-pyridyl)-2-hydroxypropionate and the compound obtained in Reference Example 25 were used as starting materials to perform the same procedure as that of Example 1 and thereby to obtain the title compound as white amorphous solid.

¹H-NMR (400 MHz, CDCl₃) δ: 1.25-2.48 (11H, m), 1.28 (3H, s), 1.41 (9H, s), 1.50 (3H, s), 1.59 (3H, s), 1.78 (3H, s), 2.34 (3H, s), 2.82-2.94 (3H, m), 3.09-3.15 (1H, m), 3.44 (1H, d, J=11.0 Hz), 3.52 (2H, brs), 3.67 (1H, d, J=11.0 Hz), 4.15 (1H, d, J=7.3 Hz), 4.23 (1H, d, J=8.5 Hz), 4.33 (1H, d, J=8.5 Hz), 4.67 (1H, s), 4.88-4.91 (2H, m), 5.23 (1H, d, J=7.6 Hz), 5.64 (1H, d, J=9.8 Hz), 5.98 (1H, d, J=5.1 Hz), 6.08 (1H, t, J=8.8 Hz), 6.20 (1H, d, J=7.8 Hz), 7.28-7.33 (1H, m), 7.42-7.49 (3H, m), 7.60 (1H, t, J=7.4 Hz), 8.13 (2H, d, J=8.3 Hz), 8.40 (1H, d, J=4.6 Hz).

### Example 23

### (1S,2S,3R,4S,5R,8R,9S,10R,13S)-4-(Acetoxy)-2-(benzoyloxy)-9,10-{(1S)-2-[(2S)-2-(dimethylcarbamoyl)azetidino]ethylidenedioxy}-5,20-epoxy-1-hydroxytax-11-en-13-yl

### (2R,3S)-3-(tert-butoxycarbonylamino)-2-hydroxy-3-(2-pyridyl)propionate

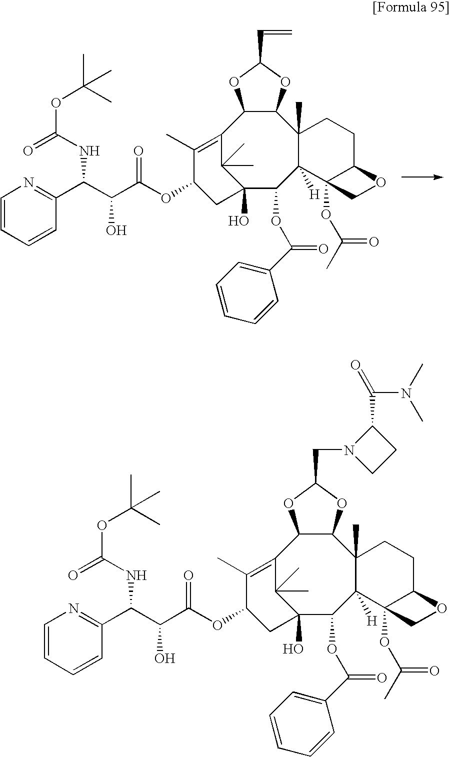

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-2-hydroxy-3-(2-pyridyl)propionate and the compound obtained in Reference Example 12, Step 2 were used as starting materials to perform the same procedure as that of Example 1 and thereby to obtain the title compound as white amorphous solid.

¹H-NMR (400 MHz, CDCl₃) δ: 1.19-2.09 (7H, m), 1.20 (3H, s), 1.45 (9H, s), 1.48 (3H, s), 1.58 (3H, s), 1.65 (3H, s), 2.28-2.36 (3H, m), 2.33 (3H, s), 2.84 (1H, dd, J=12.5, 5.6 Hz), 2.89-2.96 (2H, m), 2.91 (3H, s), 2.94 (3H, s), 3.06-3.12 (1H, m), 3.54-3.59 (1H, m), 3.95 (1H, t, J=8.4 Hz), 4.08 (1H, d, J=7.1 Hz), 4.21 (1H, d, J=8.3 Hz), 4.32 (1H, d, J=8.3 Hz), 4.89-4.92 (2H, m), 5.04 (1H, dd, J=5.4, 4.2 Hz), 5.16 (1H, d, J=6.6 Hz), 5.35 (1H, d, J=9.3 Hz), 5.95-5.98 (2H, m), 6.04 (1H, t, J=8.5 Hz), 7.22-7.25 (1H, m), 7.41-7.49 (3H, m), 7.60 (1H, t, J=7.4 Hz), 7.72 (1H, dt, J=7.7, 1.6 Hz), 8.12 (2H, d, J=7.7 Hz), 8.54 (1H, d, J=4.4 Hz).

FAB-MS; m/z: 947 (M+H)⁺.

### Example 24

### (1S,2S,3R,4S,5R,8R,9S,10R,13S)-4-(Acetoxy)-2-(benzoyloxy)-9,10-{(1S)-2-[(2S)-2-(dimethylcarbamoyl)azetidino]ethylidenedioxy}-5,20-epoxy-1-hydroxytax-11-en-13-yl

### (2R,3S)-3-(tert-butoxycarbonylamino)-3-(3-fluoro-2-pyridyl)-2-hydroxypropionate

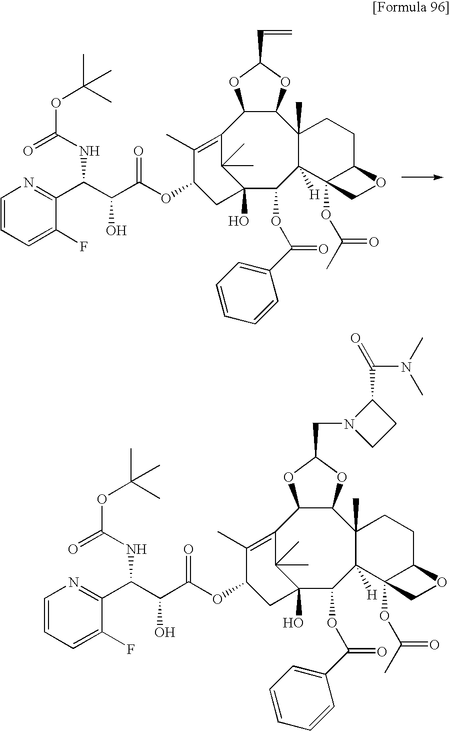

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-3-(3-fluoro-2-pyridyl)-2-hydroxypropionate and the compound obtained in Reference Example 12, Step 2 were used as starting materials to perform the same procedure as that of Example 1 and thereby to obtain the title compound as white amorphous solid.

¹H-NMR (400 MHz, CDCl₃) δ: 1.20-2.09 (7H, m), 1.23 (3H, s), 1.41 (9H, s), 1.48 (3H, s), 1.59 (3H, s), 1.73 (3H, s), 2.28-2.46 (3H, m), 2.32 (3H, s), 2.85 (1H, dd, J=12.2, 5.6 Hz), 2.90-2.96 (2H, m), 2.92 (3H, s), 2.95 (3H, s), 3.07-3.12 (1H, m), 3.55-3.59 (1H, m), 3.96 (1H, t, J=8.5 Hz), 4.10 (1H, d, J=7.1 Hz), 4.22 (1H, d, J=8.3 Hz), 4.32 (1H, d, J=8.3 Hz), 4.68 (1H, s), 4.91 (1H, s), 5.05 (1H, dd, J=5.6, 4.2 Hz), 5.18 (1H, d, J=7.1 Hz), 5.66 (1H, d, J=8.8 Hz), 5.99 (1H, d, J=5.1 Hz), 6.05 (1H, t, J=8.5 Hz), 6.20 (1H, d, J=9.0 Hz), 7.28-7.33 (1H, m), 7.42-7.49 (3H, m), 7.60 (1H, t, J=7.4 Hz), 8.12-8.14 (2H, m), 8.40 (1H, d, J=4.6 Hz).

FAB-MS; m/z: 965 (M+H)⁺.

### Example 25

### (1S,2S,3R,4S,5R,8R,9S,10R,13S)-4-(Acetoxy)-2-(benzoyloxy)-9,10-{(1S)-2-[3-(dimethylcarbamoyloxy)azetidino]ethylidenedioxy}-5,20-epoxy-1-hydroxytax-11-en-13-yl

### (2R,3S)-3-(tert-butoxycarbonylamino)-3-(3-fluoro-2-pyridyl)-2-hydroxypropionate

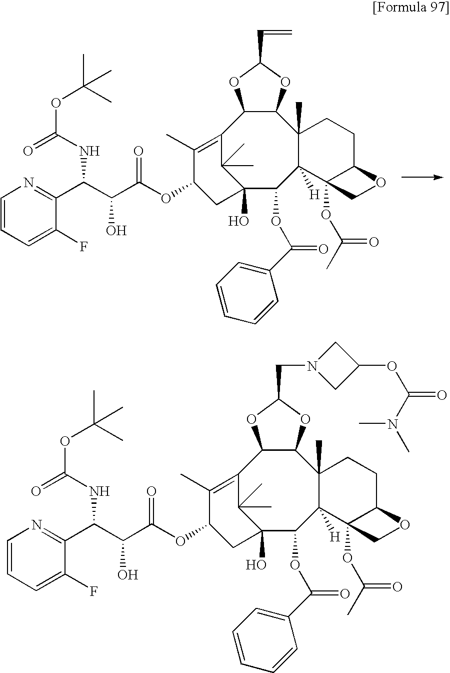

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

(2R,3S)-3-(tert-butoxycarbonylamino)-3-(3-fluoro-2-pyridyl)-2-hydroxypropionate and the compound obtained in Reference Example 13, Step 2 were used as starting materials to perform the same procedure as that of Example 1 and thereby to obtain the title compound as white amorphous solid.

¹H-NMR (400 MHz, CDCl₃) δ: 1.24-2.50 (13H, m), 1.29 (3H, s), 1.40 (9H, s), 1.50 (3H, s), 1.62 (3H, s), 1.78 (3H, s), 1.81 (3H, s), 2.77-2.98 (4H, m), 3.18-3.25 (1H, m), 3.55 (1H, d, J=2.0 Hz), 3.80-3.87 (1H, m), 4.14 (1H, d, J=6.8 Hz), 4.20-4.26 (1H, m), 4.33 (1H, d, J=8.5 Hz), 4.65-4.71 (1H, m), 4.74-4.79 (1H, m), 4.87-4.93 (1H, m), 5.02-5.38 (3H, m), 5.64 (1H, d, J=8.3 Hz), 5.98-6.00 (1H, m), 6.05-6.13 (1H, m), 6.17-6.24 (1H, m), 7.28-7.34 (1H, m), 7.42-7.50 (3H, m), 7.60 (1H, t, J=7.3 Hz), 8.13 (2H, d, J=7.3 Hz), 8.41 (1H, d, J=4.6 Hz).

FAB-MS; m/z: 981 (M+H)⁺.

### Example 26

### (1S,2S,3R,4S,5R,8R,9S,10R,13S)-4-(Acetoxy)-2-(benzoyloxy)-9,10-[(1S)-2-(3-carbamoyloxy)azetidino]ethylidenedioxy]-5,20-epoxy-1-hydroxytax-11-en-13-yl

### (2R,3S)-3-(tert-butoxycarbonylamino)-3-(3-fluoro-2-pyridyl)-2-hydroxypropionate

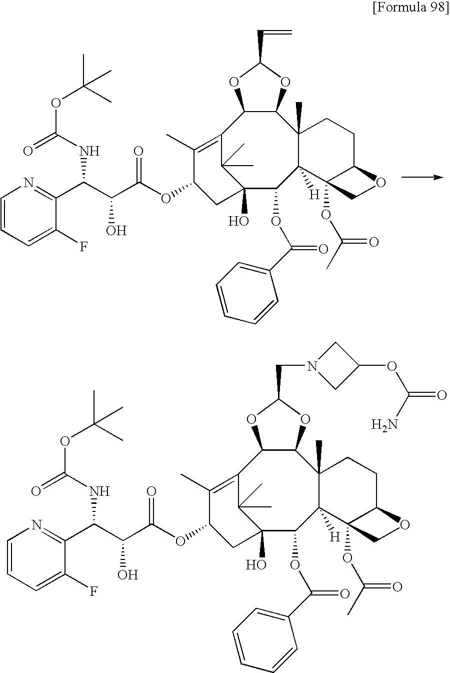

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-3-(3-fluoro-2-pyridyl)-2-hydroxypropionate and the compound obtained in Reference Example 14, Step 2 were used as starting materials to perform the same procedure as that of Example 1 and thereby to obtain the title compound as white amorphous solid.

¹H-NMR (400 MHz, CDCl₃) δ: 1.20-2.49 (8H, m), 1.28 (3H, s), 1.40 (9H, s), 1.50 (3H, s), 1.61 (3H, s), 1.78 (3H, s), 2.34 (3H, s), 2.80 (1H, dd, J=12.5, 5.9 Hz), 2.88-2.95 (2H, m), 3.24-3.28 (2H, m), 3.80 (2H, q, J=6.8 Hz), 4.09-4.15 (1H, m), 4.23 (1H, d, J=8.5 Hz), 4.33 (1H, d, J=8.1 Hz), 4.68 (1H, d, J=2.7 Hz), 4.70 (2H, brs), 4.87-4.93 (2H, m), 5.04-5.11 (1H, m), 5.22 (1H, d, J=7.1 Hz), 5.64 (1H, d, J=8.5 Hz), 5.99 (1H, d, J=5.1 Hz), 6.08 (1H, t, J=8.3 Hz), 6.21 (1H, d, J=9.0 Hz), 7.28-7.33 (1H, m), 7.42-7.50 (3H, m), 7.60 (1H, t, J=7.4 Hz), 8.13 (2H, d, J=7.3 Hz), 8.40 (1H, d, J=4.6 Hz).

FAB-MS; m/z: 953 (M+H)⁺.

### Example 27

### (1S,2S,3R,4S,5R,8R,9S,10R,13S)-4-(Acetoxy)-2-(benzoyloxy)-5,20-epoxy-1-hydroxy-9,10-{(1S)-2-[3-(N-methoxycarbonyl-N-methylamino)azetidino]ethylidenedioxy}tax-11-en-13-yl (2R,3S)-3-(tert-butoxycarbonylamino)-3-(3-fluoro-2-pyridyl)-2-hydroxypropionate

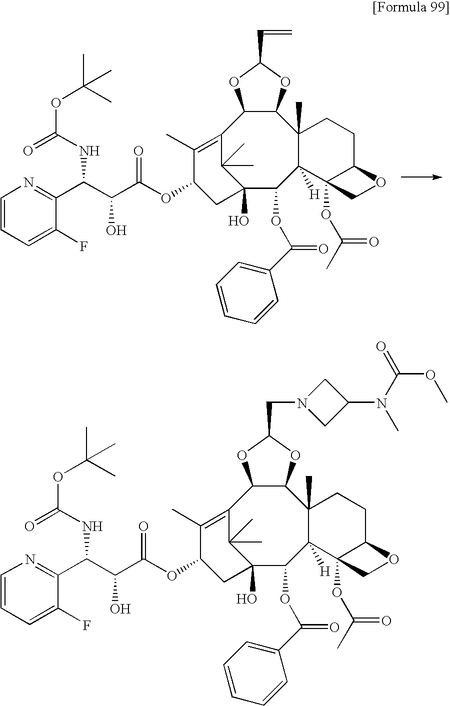

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-3-(3-fluoro-2-pyridyl)-2-hydroxypropionate and the compound obtained in Reference Example 15, Step 2 were used as starting materials to perform the same procedure as that of Example 1 and thereby to obtain the title compound as white amorphous solid.

¹H-NMR (400 MHz, CDCl₃) δ: 1.23-2.49 (12H, m), 1.29 (3H, s), 1.40 (9H, s), 1.50 (3H, s), 1.62 (3H, s), 1.78 (3H, s), 1.81 (3H, s), 2.75-2.95 (1H, m), 2.96 (3H, s), 3.24 (1H, brs), 3.55 (1H, d, J=1.7 Hz), 3.70 (3H, s), 4.14 (1H, d, J=7.3 Hz), 4.20-4.26 (1H, m), 4.33 (1H, d, J=8.5 Hz), 4.66-4.93 (4H, m), 5.20-5.37 (1H, m), 5.64 (1H, d, J=8.8 Hz), 5.99 (1H, d, J=4.9 Hz), 6.06-6.13 (1H, m), 6.18-6.25 (1H, m), 7.28-7.33 (1H, m), 7.42-7.50 (3H, m), 7.60 (1H, t, J=7.3 Hz), 8.13 (2H, d, J=7.6 Hz), 8.41 (1H, d, J=4.6 Hz).

### Example 28

### (1S,2S,3R,4S,5R,8R,9S,10R,13S)-4-(Acetoxy)-2-(benzoyloxy)-5,20-epoxy-1-hydroxy-9,10-{(1S)-2-[3-(methanesulfonyl)azetidino]ethylidenedioxy}tax-11-en-13-yl

### (2R,3S)-3-(tert-butoxycarbonylamino)-3-(3-fluoro-2-pyridyl)-2-hydroxypropionate

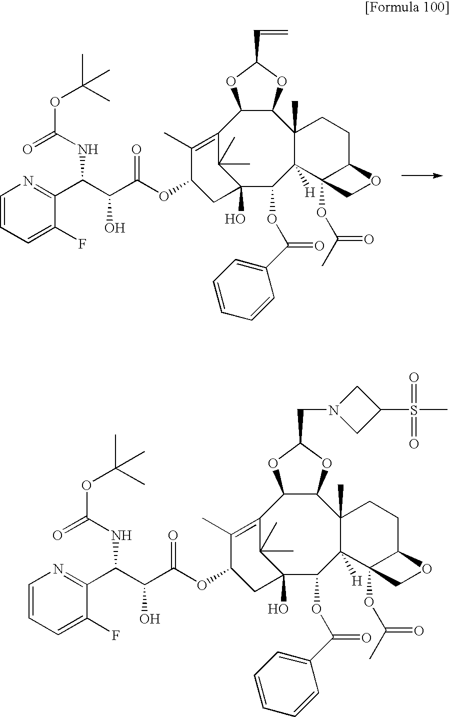

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-3-(3-fluoro-2-pyridyl)-2-hydroxypropionate and the compound obtained in Reference Example 16, Step 3 were used as starting materials to perform the same procedure as that of Example 1 and thereby to obtain the title compound as white amorphous solid.

¹H-NMR (400 MHz, CDCl₃) δ: 1.26-2.14 (7H, m), 1.29 (3H, s), 1.40 (9H, s), 1.50 (3H, s), 1.60 (3H, s), 1.78 (3H, s), 2.35 (3H, s), 2.45 (1H, dd, J=14.4, 9.5 Hz), 2.83 (1H, dd, J=12.5, 5.6 Hz), 2.88-2.93 (2H, m), 2.95 (3H, s), 3.68-3.79 (4H, m), 3.88-3.95 (1H, m), 4.14 (1H, d, J=7.3 Hz), 4.23 (1H, d, J=8.5 Hz), 4.33 (1H, d, J=8.5 Hz), 4.67 (1H, d, J=2.2 Hz), 4.86 (1H, t, J=4.9 Hz), 4.91 (1H, s), 5.21 (1H, d, J=6.8 Hz), 5.64 (1H, d, J=8.5 Hz), 5.98 (1H, d, J=4.9 Hz), 6.09 (1H, t, J=8.2 Hz), 6.21 (1H, d, J=8.8 Hz), 7.28-7.33 (1H, m), 7.43-7.49 (3H, m), 7.60 (1H, t, J=7.4 Hz), 8.14 (2H, d, J=7.4 Hz), 8.40 (1H, d, J=4.6 Hz).

FAB-MS; m/z: 972 (M+H)⁺.

### Example 29

### (1S,2S,3R,4S,5R,8R,9S,10R,13S)-4-(Acetoxy)-2-(benzoyloxy)-9,10-{(1S)-2-[3-(dimethylamino)azetidino]ethylidenedioxy}-5,20-epoxy-1-hydroxytax-11-en-13-yl

### (2R,3S)-3-(tert-butoxycarbonylamino)-2-hydroxy-3-(3-fluoro-2-pyridyl)propionate

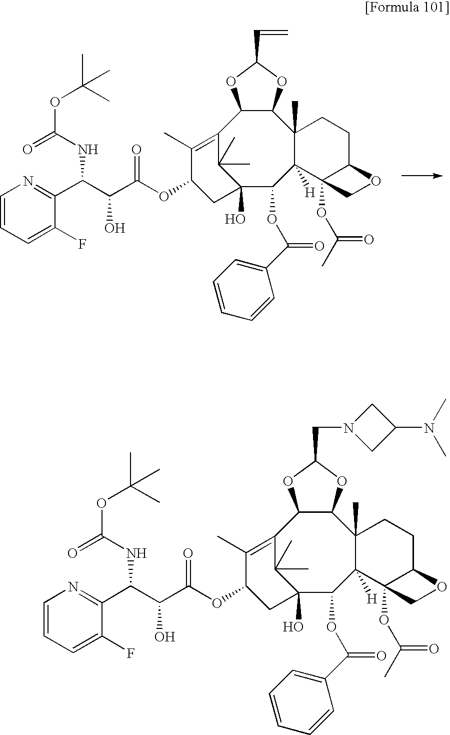

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-3-(3-fluoro-2-pyridyl)-2-hydroxypropionate and 3-(N,N-dimethylamino)azetidine hydrochloride (J. Med. Chem., 1993, 7, 801) were used as starting materials to perform the same procedure as that of Example 1 and thereby to obtain the title compound as white amorphous solid.

¹H-NMR (400 MHz, CDCl₃) δ: 1.24-2.16 (7H, m), 1.28 (3H, s), 1.40 (9H, s), 1.48 (3H, s), 1.61 (3H, s), 1.78 (3H, s), 2.13 (6H, s), 2.34 (3H, s), 2.41-2.48 (1H, m), 2.81 (1H, dd, J=12.5, 5.6 Hz), 2.86-2.96 (3H, m), 3.04 (2H, q, J=7.3 Hz), 3.62-3.66 (2H, m), 4.13 (1H, d, J=7.3 Hz), 4.23 (1H, d, J=8.3 Hz), 4.32 (1H, d, J=8.3 Hz), 4.67 (1H, d, J=2.7 Hz), 4.87-4.91 (2H, m), 5.21 (1H, d, J=7.1 Hz), 5.65 (1H, d, J=8.8 Hz), 5.98 (1H, d, J=5.1 Hz), 6.08 (1H, t, J=8.8 Hz), 6.21 (1H, d, J=9.0 Hz), 7.28-7.32 (1H, m), 7.42-7.49 (3H, m), 7.60 (1H, t, J=7.4 Hz), 8.12-8.15 (2H, m), 8.40 (1H, d, J=4.6 Hz).

MS (FAB)m/z: 937 (M+H)⁺.

### Example 30

### (1S,2S,3R,4S,5R,8R,9S,10R,13S)-4-(Acetoxy)-2-(benzoyloxy)-5,20-epoxy-1-hydroxy-9,10-[(1S)-2-(methoxyamino-3-ylideneazetidino)ethylidenedioxy]tax-11-en-13-yl

### (2R,3S)-3-(tert-butoxycarbonylamino)-3-(3-fluoro-2-pyridyl)-2-hydroxypropionate

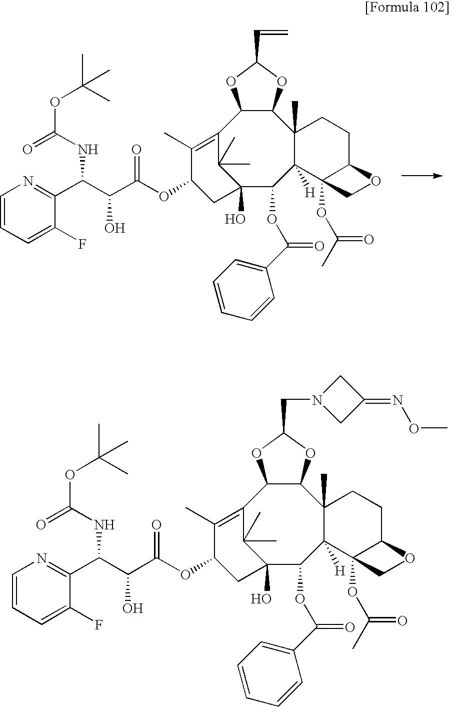

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-3-(3-fluoro-2-pyridyl)-2-hydroxypropionate and the compound obtained in Reference Example 17, Step 2 were used as starting materials to perform the same procedure as that of Example 1 and thereby to obtain the title compound as white amorphous solid.

¹H-NMR (400 MHz, CDCl₃) δ: 0.98-2.53 (7H, m), 1.29 (3H, s), 1.40 (9H, s), 1.50 (3H, s), 1.61 (3H, s), 1.79 (3H, s), 2.35 (3H, s), 2.91-2.97 (2H, m), 3.01 (1H, dd, J=15.4, 4.2 Hz), 3.84 (1H, d, J=5.1 Hz), 3.86 (3H, s), 4.15-4.17 (1H, m), 4.15 (4H, s), 4.23 (1H, d, J=8.3 Hz), 4.33 (1H, d, J=8.3 Hz), 4.68 (1H, d, J=2.4 Hz), 4.89-4.93 (2H, m), 5.24 (1H, d, J=7.3 Hz), 5.64 (1H, d, J=9.0 Hz), 5.98 (1H, d, J=5.1 Hz), 6.09 (1H, t, J=8.5 Hz), 6.21 (1H, d, J=8.5 Hz), 7.28-7.33 (1H, m), 7.42-7.50 (3H, m), 7.60 (1H, t, J=7.4 Hz), 8.14 (2H, d, J=7.3 Hz), 8.40 (1H, d, J=4.6 Hz).

FAB-MS; m/z: 937 (M+H)⁺.

### Example 31

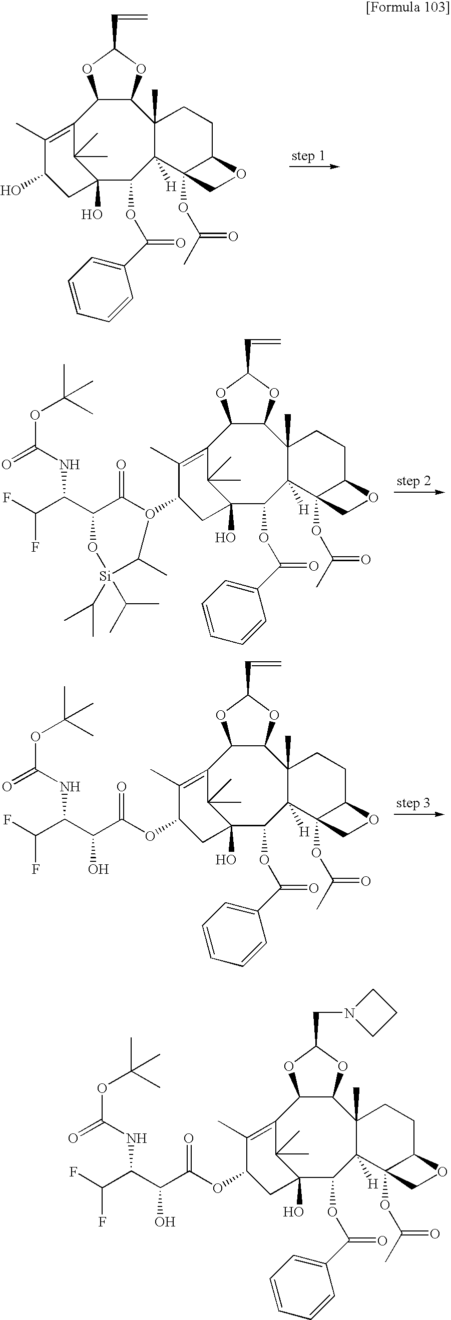

### Step 1: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3R)-3-(tert-butoxycarbonylamino)-4,4-difluoro-2-[(triisopropylsilyl)oxy]butyrate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-11-ene (Bioorg. Med. Chem. Lett., 2003, 13, 185, 1.00 g) and (3R,4R)-1-(tert-butoxycarbonyl)-4-(difluoromethyl)-3-[(triisopropylsilyl)oxy]-2-azetidin one (Bioorg. Med. Chem., 2000, 8, 1619, 900 mg) were dissolved in tetrahydrofuran (35.0 ml) under an argon flow, and the solution was added with lithium bis(trimethylsilyl)amide (1 mole solution in tetrahydrofuran, 2.32 ml) at −78° C., and stirred at 0° C. for 30 minutes. The reaction mixture was added with saturated aqueous ammonium chloride, and extracted with ethyl acetate. The organic layer was washed with saturated brine, and dried, and then the solvent was evaporated under reduced pressure. The resulting residue was purified by silica gel chromatography [developing solvent; ethyl acetate:n-hexane=1:9 to 1:4 (v/v)] to obtain the title compound (1.22 g) as white amorphous solid.

¹H-NMR (400 MHz, CDCl₃) δ: 1.08-1.20 (21H, m), 1.24-2.04 (4H, m), 1.29 (3H, s), 1.38 (9H, s), 1.51 (3H, s), 1.64 (3H, s), 1.78 (3H, s), 2.23-2.30 (3H, m), 2.35 (3H, s), 2.93 (1H, d, J=4.9 Hz), 4.19-4.21 (2H, m), 4.33-4.39 (2H, m), 4.87 (1H, s), 4.92 (1H, s), 5.03 (1H, d, J=10.7 Hz), 5.20 (1H, d, J=6.3 Hz), 5.26 (1H, d, J=6.6 Hz), 5.45 (1H, d, J=10.5 Hz), 5.57 (1H, d, J=17.3 Hz), 5.78 (1H, dt, J=5.9, 56.2 Hz), 5.95-6.04 (3H, m), 7.50 (2H, t, J=7.6 Hz), 7.61 (1H, t, J=7.6 Hz), 8.15 (2H, d, J=7.6 Hz).

FAB-MS; m/z: 962 (M+H)⁺, 984 (M+Na)⁺.

### Step 2: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3R)-3-(tert-butoxycarbonylamino)-4,4-difluoro-2-hydroxybutyrate

The compound obtained in Step 1 mentioned above (1.12 g) was dissolved in tetrahydrofuran (23 ml), and the solution was added with tetra-n-butylammonium fluoride (1 mole solution in tetrahydrofuran, 2.33 ml) at 0° C., and stirred for 10 minutes. The reaction mixture was added with saturated brine, and extracted with ethyl acetate. The organic layer was dried, and then the solvent was evaporated under reduced pressure. The resulting residue was purified by silica gel chromatography [developing solvent; ethyl acetate:n-hexane=1:2 (v/v)] to obtain the title compound (893 mg) as white amorphous solid.

¹H-NMR (400 MHz, CDCl₃) δ: 1.24-2.11 (6H, m), 1.25 (3H, s), 1.42 (9H, s), 1.50 (3H, s), 1.64 (3H, s), 1.79 (3H, s), 2.24-2.39 (1H, m), 2.34 (3H, s), 2.94 (1H, d, J=4.9 Hz), 4.18 (1H, d, J=7.1 Hz), 4.25 (1H, d, J=8.5 Hz), 4.34 (1H, d, J=8.5 Hz), 4.51-4.75 (3H, m), 4.94 (1H, s), 5.22-5.26 (2H, m), 5.32 (1H, d, J=6.8 Hz), 5.47 (1H, d, J=10.5 Hz), 5.58 (1H, d, J=17.3 Hz), 5.76-6.05 (3H, m), 6.13 (1H, t, J=8.3 Hz), 7.48 (2H, t, J=7.7 Hz), 7.61 (1H, t, J=7.7 Hz), 8.13 (2H, d, J=7.7 Hz).

FAB-MS; m/z: 806 (M+H)⁺, 828 (M+Na)⁺.

### Step 3: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-1′-en-13-yl

### (2R,3R)-3-(tert-butoxycarbonylamino)-4,4-difluoro-2-hydroxybutyrate

The compound obtained in Step 2 mentioned above was used as a starting material to perform the same procedure as that of Example 1 and thereby to obtain the title compound as white amorphous solid.

¹H-NMR (400 MHz, CDCl₃) δ: 1.23-2.18 (9H, m), 1.23 (3H, s), 1.42 (9H, s), 1.49 (3H, s), 1.61 (3H, s), 1.77 (3H, s), 2.32-2.38 (1H, m), 2.33 (3H, s), 2.76 (1H, dd, J=12.6, 5.9 Hz), 2.84 (1H, dd, J=12.6, 4.3 Hz), 2.92 (1H, d, J=4.9 Hz), 3.36-3.40 (4H, m), 4.09-4.13 (1H, m), 4.25 (1H, d, J=8.5 Hz), 4.33 (1H, d, J=8.5 Hz), 4.49-4.58 (1H, m), 4.64 (1H, s), 4.87-4.90 (1H, m), 4.93 (1H, s), 5.21-5.26 (2H, m), 5.75-6.04 (2H, m), 6.11 (1H, t, J=8.5 Hz), 7.48 (2H, t, J=7.6 Hz), 7.61 (1H, t, J=7.6 Hz), 8.13 (2H, d, J=7.6 Hz).

FAB-MS; m/z: 849 (M+H)⁺.

### Example 32

### (1S,2S,3R,4S,5R,8R,9S,10R,13S)-4-(Acetoxy)-2-(benzoyloxy)-5,20-epoxy-1-hydroxy-9,10-[(1S)-2-(3-hydroxyazetidino)ethylidenedioxy]tax-11-en-13-yl

### (2R,3R)-3-(tert-butoxycarbonylamino)-4,4-difluoro-2-hydroxybutyrate

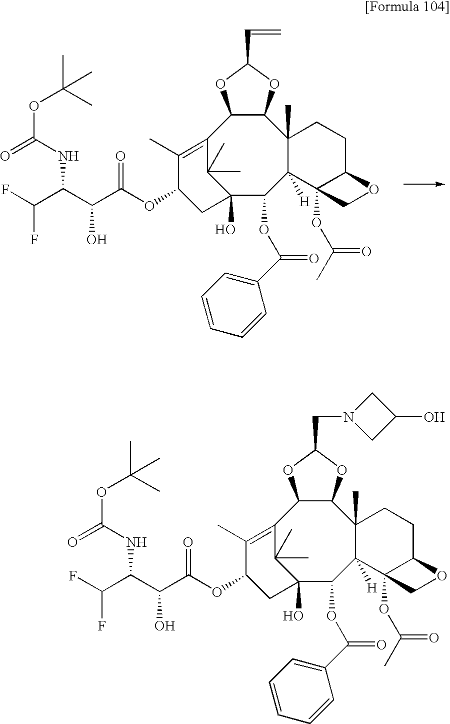

The compound obtained in Example 31, Step 2 and 3-azetidinol hydrochloride were used as starting materials to perform the same procedure as that of Example 1 and thereby to obtain the title compound as white amorphous solid.

¹H-NMR (400 MHz, CDCl₃) δ: 1.23-2.38 (10H, m), 1.24 (3H, s), 1.42 (9H, s), 1.49 (3H, s), 1.60 (3H, s), 1.77 (3H, s), 2.34 (3H, s), 2.82 (1H, dd, J=12.5, 5.4 Hz), 2.90-2.94 (2H, m), 3.16-3.21 (2H, m), 3.77-3.82 (2H, m), 4.12 (1H, d, J=6.8 Hz), 4.24 (1H, d, J=8.5 Hz), 4.34 (1H, d, J=8.5 Hz), 4.45-4.60 (2H, m), 4.65 (1H, s), 4.89-4.93 (2H, m), 5.22-5.24 (1H, m), 5.90 (1H, dt, J=55.9, 4.6 Hz), 6.00 (1H, d, J=5.6 Hz), 6.11 (1H, t, J=8.1 Hz), 7.49 (2H, t, J=7.6 Hz), 7.62 (1H, t, J=7.6 Hz), 8.12-8.14 (2H, m).

FAB-MS; m/z: 865 (M+H)⁺.

### Example 33

### (1S,2S,3R,4S,5R,8R,9S,10R,13S)-4-(Acetoxy)-2-(benzoyloxy)-5,20-epoxy-1-hydroxy-9,10-[(1S)-2-(3-hydroxy-3-methylazetidino)ethylidenedioxy]tax-11-en-13-yl

### (2R,3R)-3-(tert-butoxycarbonylamino)-4,4-difluoro-2-hydroxybutyrate

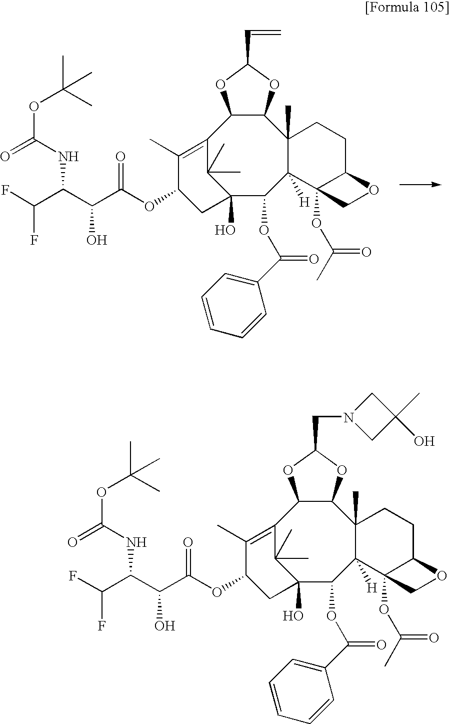

The compound obtained in Example 31, Step 2 and the compound obtained in Reference Example 3 were used as starting materials to perform the same procedure as that of Example 1 and thereby to obtain the title compound as white amorphous solid.

¹H-NMR (400 MHz, CDCl₃) δ: 1.21-2.06 (9H, m), 1.24 (3H, s), 1.42 (9H, s), 1.49 (3H, s), 1.52 (3H, s), 1.61 (3H, s), 1.76 (3H, s), 2.32-2.38 (1H, m), 2.34 (3H, s), 2.80 (1H, dd, J=12.0, 5.6 Hz), 2.90-2.94 (2H, m), 3.20-3.24 (2H, m), 3.42-3.46 (2H, m), 4.12 (1H, d, J=7.1 Hz), 4.24 (1H, d, J=8.3 Hz), 4.34 (1H, d, J=8.3 Hz), 4.49-4.58 (1H, m), 4.65 (1H, s), 4.90-4.93 (2H, m), 5.21-5.26 (1H, m), 5.90 (1H, dt, J=55.7, 5.6 Hz), 6.00 (1H, d, J=4.9 Hz), 6.11 (1H, t, J=8.8 Hz), 7.49 (2H, t, J=7.6 Hz), 7.62 (1H, t, J=7.6 Hz), 8.12-8.14 (2H, m).

FAB-MS; m/z: 879 (M+H)⁺.

### Example 34

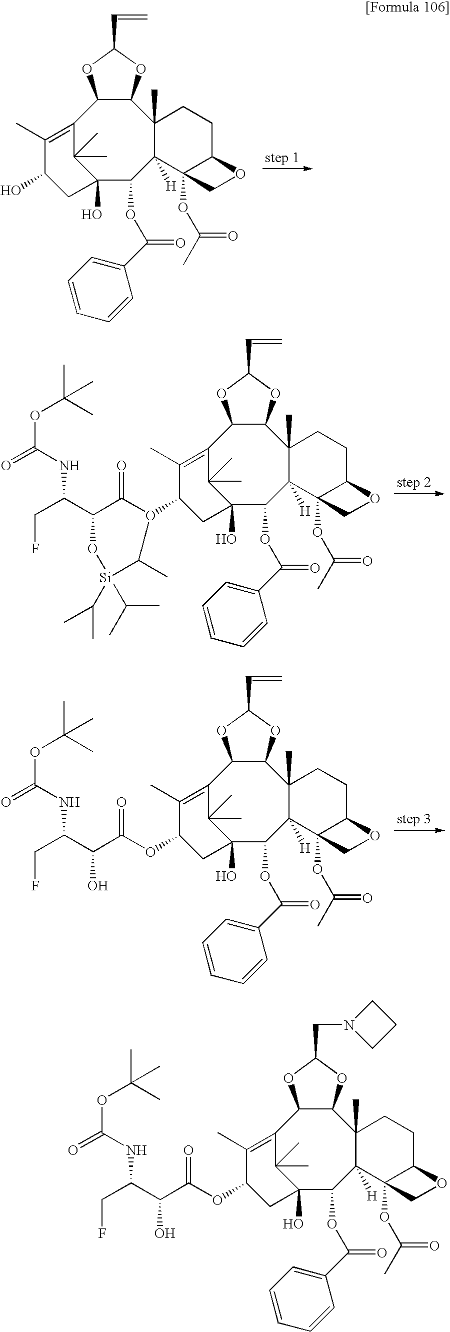

### Step 1: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3R)-3-(tert-butoxycarbonylamino)-4-fluoro-2-[(triisopropylsilyl)oxy]butyrate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-11-ene and the compound obtained in Reference Example 18, Step 4 were used as starting materials to perform the same procedure as that of Example 31, Step 1 and thereby to obtain the title compound as white amorphous solid.

¹H-NMR (400 MHz, CDCl₃) δ: 1.08-2.05 (26H, m), 1.29 (3H, s), 1.37 (9H, s), 1.51 (3H, s), 1.64 (3H, s), 1.79 (3H, s), 2.24-2.28 (2H, m), 2.33 (3H, s), 2.94 (1H, d, J=5.4 Hz), 4.18-4.21 (2H, m), 4.33-4.48 (4H, m), 4.77-4.79 (2H, m), 4.92 (1H, s), 5.20 (1H, d, J=6.1 Hz), 5.27 (1H, d, J=7.1 Hz), 5.46 (1H, d, J=10.5 Hz), 5.58 (1H, d, J=17.3 Hz), 5.96-6.04 (3H, m), 7.49 (2H, t, J=7.6 Hz), 7.61 (1H, t, J=7.4 Hz), 8.14-8.16 (2H, m).

FAB-MS; m/z: 944 (M+H)⁺, 966 (M+Na)⁺.

### Step 2: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3R)-3-(tert-butoxycarbonylamino)-4-fluoro-2-hydroxybutyrate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.20-2.25 (7H, m), 1.25 (3H, s), 1.42 (9H, s), 1.50 (3H, s), 1.64 (3H, s), 1.81 (3H, s), 2.33 (3H, s), 2.35-2.42 (1H, m), 2.94 (1H, d, J=4.6 Hz), 4.18 (1H, d, J=7.1 Hz), 4.25 (1H, d, J=8.5 Hz), 4.34 (1H, d, J=8.5 Hz), 4.42-4.56 (4H, m), 4.94 (1H, s), 5.12 (1H, d, J=10.0 Hz), 5.25 (1H, d, J=6.3 Hz), 5.32 (1H, d, J=7.3 Hz), 5.47 (1H, d, J=10.5 Hz), 5.58 (1H, d, J=17.1 Hz), 5.97-6.11 (3H, m), 7.48 (2H, t, J=7.6 Hz), 7.59-7.63 (1H, m), 8.12-8.14 (2H, m).

FAB-MS; m/z: 788 (M+H)⁺, 810 (M+Na)⁺.

### Step 3: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3R)-3-(tert-butoxycarbonylamino)-4-fluoro-2-hydroxybutyrate

The compound obtained in Step 2 mentioned above was used as a starting material to perform the same procedure as that of Example 1 and thereby to obtain the title compound as white amorphous solid.

¹H-NMR (400 MHz, CDCl₃) δ: 1.18-2.40 (10H, m), 1.24 (3H, s), 1.41 (9H, s), 1.49 (3H, s), 1.61 (3H, s), 1.78 (3H, s), 2.33 (3H, s), 2.74 (1H, dd, J=12.5, 5.6 Hz), 2.83 (1H, dd, J=12.5, 4.4 Hz), 2.92 (1H, d, J=4.9 Hz), 3.32-3.40 (4H, m), 4.12 (1H, d, J=7.1 Hz), 4.24 (1H, d, J=8.5 Hz), 4.33 (1H, d, J=8.5 Hz), 4.41-4.61 (4H, m), 4.86-4.89 (1H, m), 4.93 (1H, s), 5.17 (1H, d, J=9.0 Hz), 5.22 (1H, d, J=6.8 Hz), 6.00 (1H, d, J=5.1 Hz), 6.06 (1H, t, J=8.5 Hz), 7.48 (2H, t, J=7.6 Hz), 7.61 (1H, t, J=7.6 Hz), 8.13 (2H, d, J=7.6 Hz).

FAB-MS; m/z: 831 (M+H)⁺.

### Example 35

### (1S,2S,3R,4S,5R,8R,9S,10R,13S)-4-(Acetoxy)-2-(benzoyloxy)-5,20-epoxy-1-hydroxy-9,10-[(1S)-2-(3-hydroxyazetidino)ethylidenedioxy]tax-11-en-13-yl

### (2R,3R)-3-(tert-butoxycarbonylamino)-4-fluoro-2-hydroxybutyrate

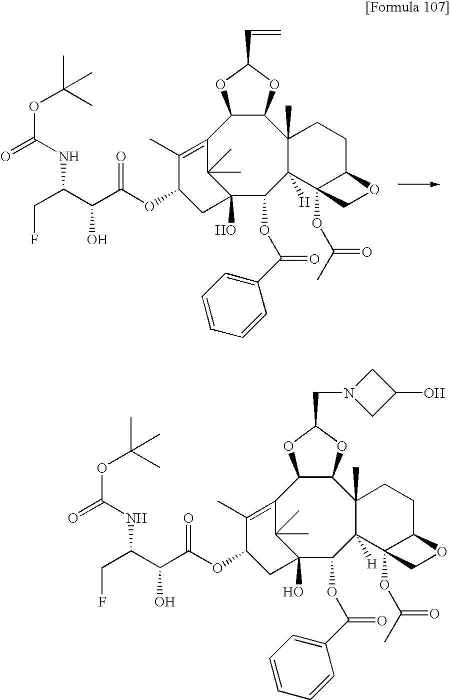

The compound obtained in Example 34, Step 2 and 3-azetidinol hydrochloride were used as starting materials to perform the same procedure as that of Example 1 and thereby to obtain the title compound as white amorphous solid.

¹H-NMR (400 MHz, CDCl₃) δ: 1.23-2.40 (9H, m), 1.24 (3H, s), 1.41 (9H, s), 1.49 (3H, s), 1.61 (3H, s), 1.79 (3H, s), 2.33 (3H, s), 2.79 (1H, dd, J=12.5, 5.6 Hz), 2.87-2.93 (2H, m), 3.08-3.14 (2H, m), 3.75-3.79 (2H, m), 4.12 (1H, d, J=7.1 Hz), 4.24 (1H, d, J=8.5 Hz), 4.32 (1H, d, J=8.5 Hz), 4.41-4.55 (5H, m), 4.89 (1H, dd, J=5.4, 4.6 Hz), 4.93 (1H, s), 5.14 (1H, d, J=9.0 Hz), 5.23 (1H, d, J=7.1 Hz), 6.00 (1H, d, J=5.1 Hz), 6.07 (1H, t, J=8.3 Hz), 7.48 (2H, t, J=7.7 Hz), 7.59-7.63 (1H, m), 8.11-8.14 (2H, m).

FAB-MS; m/z: 847 (M+H)⁺.

### Example 36

### (1S,2S,3R,4S,5R,8R,9S,10R,13S)-4-(Acetoxy)-2-(benzoyloxy)-5,20-epoxy-1-hydroxy-9,10-[(1S)-2-(3-hydroxy-3-methylazetidino)ethylidenedioxy]tax-11-en-13-yl

### (2R,3R)-3-(tert-butoxycarbonylamino)-4-fluoro-2-hydroxybutyrate

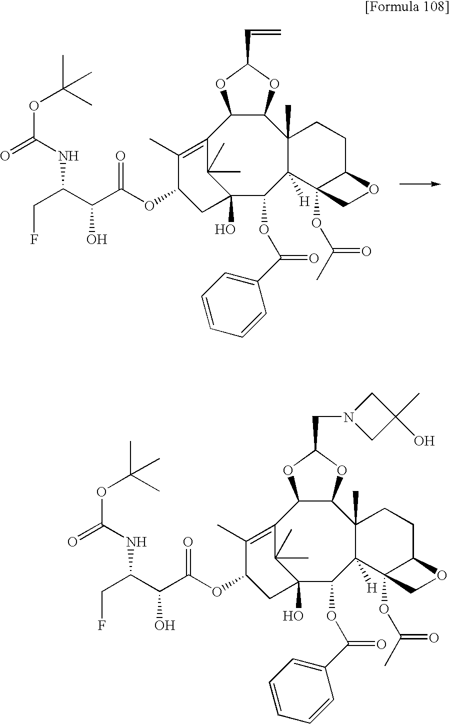

The compound obtained in Example 34, Step 2 and the compound obtained in Reference Example 3 were used as starting materials to perform the same procedure as that of Example 1 and thereby to obtain the title compound as white amorphous solid.

¹H-NMR (400 MHz, CDCl₃) δ: 1.23-2.05 (11H, m), 1.24 (3H, s), 1.41 (9H, s), 1.49 (3H, s), 1.60 (3H, s), 1.78 (3H, s), 2.32-2.40 (1H, m), 2.33 (3H, s), 2.79 (1H, dd, J=12.5, 5.6 Hz), 2.89-2.93 (2H, m), 3.22 (2H, t, J=8.1 Hz), 3.43 (2H, t, J=8.1 Hz), 4.12 (1H, d, J=7.1 Hz), 4.24 (1H, d, J=8.5 Hz), 4.33 (1H, d, J=8.5 Hz), 4.41-4.64 (4H, m), 4.91 (1H, dd, J=5.6, 4.6 Hz), 4.93 (1H, s), 5.15 (1H, d, J=9.3 Hz), 5.23 (1H, d, J=6.8 Hz), 6.00 (1H, d, J=5.1 Hz), 6.07 (1H, t, J=8.1 Hz), 7.48 (2H, t, J=7.8 Hz), 7.59-7.63 (1H, m), 8.12-8.14 (2H, m).

FAB-MS; m/z: 861 (M+H)⁺.

### Example 37

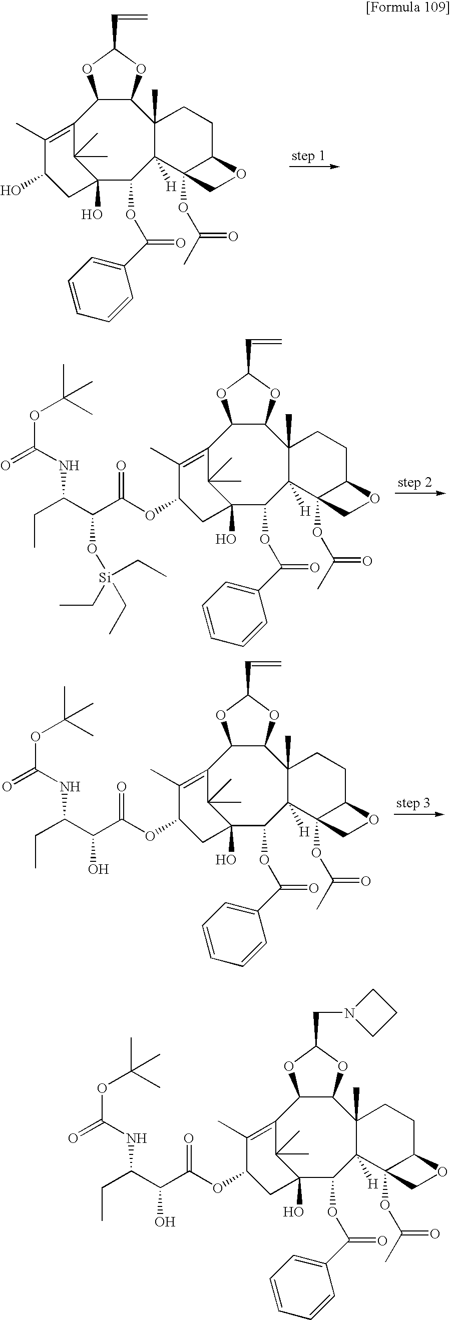

### Step 1: (1S,2S,3R,4S,5R,8R,9S,10R,13S)-4-(Acetoxy)-2-(benzoyloxy)-5,20-epoxy-1-hydroxy-9,10-[(1S)-(2-propenylidenedioxy)]tax-11-en-13-yl

### (2R,3S)-3-(tert-butoxycarbonylamino)-2-[(triethylsilyl)oxy]pentanoate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-11-ene and the compound obtained in Reference Example 19, Step 3 were used as starting materials to perform the same procedure as that of Example 31, Step 1 and thereby to obtain the title compound as white amorphous solid.

¹H-NMR (400 MHz, CDCl₃) δ: 0.59-0.72 (6H, m), 0.95-2.38 (21H, m), 1.30 (3H, s), 1.37 (9H, s), 1.51 (3H, s), 1.64 (3H, s), 1.78 (3H, s), 2.36 (3H, s), 2.93 (1H, d, J=5.1 Hz), 3.91-3.99 (1H, m), 4.19-4.22 (2H, m), 4.29-4.36 (2H, m), 4.78 (1H, d, J=10.3 Hz), 4.94 (1H, s), 5.21 (1H, d, J=6.1 Hz), 5.27 (1H, d, J=6.8 Hz), 5.45 (1H, d, J=10.0 Hz), 5.58 (1H, d, J=17.3 Hz), 5.95-6.06 (2H, m), 7.49 (2H, t, J=7.7 Hz), 7.61 (1H, t, J=7.3 Hz), 8.13-8.16 (2H, m).

### Step 2: (1S,2S,3R,4S,5R,8R,9S,10R,13S)-4-(Acetoxy)-2-(benzoyloxy)-5,20-epoxy-1-hydroxy-9,10-[(1S)-(2-propenylidenedioxy)]tax-11-en-13-yl

### (2R,3S)-3-(tert-butoxycarbonylamino)-2-hydroxypentanoate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 0.90-2.42 (12H, m), 1.26 (3H, s), 1.40 (9H, s), 1.50 (3H, s), 1.64 (3H, s), 1.81 (3H, s), 2.33 (3H, s), 2.94 (1H, d, J=5.1 Hz), 3.98-4.07 (2H, m), 4.19 (1H, d, J=6.8 Hz), 4.24 (1H, d, J=8.3 Hz), 4.32-4.35 (2H, m), 4.83 (1H, d, J=9.8 Hz), 4.93-5.00 (1H, m), 5.25 (1H, d, J=6.1 Hz), 5.31 (1H, d, J=7.3 Hz), 5.47 (1H, d, J=10.3 Hz), 5.58 (1H, d, J=17.3 Hz), 5.96-6.10 (3H, m), 7.48 (2H, t, J=7.7 Hz), 7.61 (1H, t, J=7.4 Hz), 8.12-8.15 (2H, m).

FAB-MS; m/z: 944 (M+H)⁺, 966 (M+Na)⁺.

### Step 3: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxy-tax-11-en-13-yl

### (2R,3S)-3-(tert-butoxycarbonylamino)-2-hydroxypentanoate

The compound obtained in Step 2 mentioned above was used as a starting material to perform the same procedure as that of Example 1 and thereby to obtain the title compound as white amorphous solid.

¹H-NMR (400 MHz, CDCl₃) δ: 0.97-2.42 (16H, m), 1.00 (3H, t, J=7.3 Hz), 1.24 (3H, s), 1.39 (9H, s), 1.49 (3H, s), 1.62 (3H, d, J=9.3 Hz), 1.78 (3H, s), 2.33 (3H, s), 2.75 (1H, dd, J=12.5, 5.6 Hz), 2.83 (1H, dd, J=12.5, 4.4 Hz), 2.92 (1H, d, J=4.9 Hz), 3.32-3.41 (1H, m), 3.98-4.05 (1H, m), 4.12 (1H, d, J=7.1 Hz), 4.24 (1H, d, J=8.3 Hz), 4.31-4.35 (2H, m), 4.83-4.89 (1H, m), 4.93 (1H, s), 5.22 (1H, d, J=6.8 Hz), 6.00 (1H, d, J=5.1 Hz), 6.05 (1H, t, J=8.3 Hz), 7.48 (2H, t, J=7.8 Hz), 7.61 (1H, t, J=7.4 Hz), 8.11-8.15 (2H, m).

FAB-MS; m/z: 827 (M+H)⁺.

### Example 38

### (1S,2S,3R,4S,5R,8R,9S,10R,13S)-4-(Acetoxy)-2-(benzoyloxy)-5,20-epoxy-1-hydroxy-9,10-[(1S)-2-(3-hydroxyazetidino)ethylidenedioxy]tax-11-en-13-yl

### (2R,3S)-3-(tert-butoxycarbonylamino)-2-hydroxypentanoate

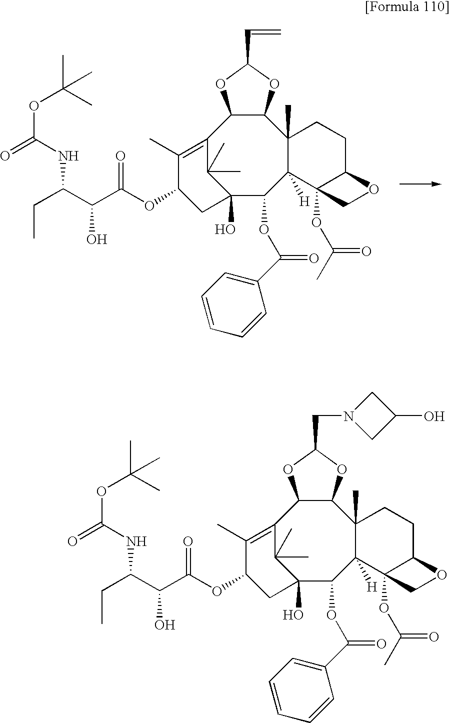

The compound obtained in Example 37, Step 2 and 3-azetidinol hydrochloride were used as starting materials to perform the same procedure as that of Example 1 and thereby to obtain the title compound as white amorphous solid.

¹H-NMR (400 MHz, CDCl₃) δ: 1.01 (3H, t, J=7.3 Hz), 1.17-2.41 (10H, m), 1.25 (3H, s), 1.39 (9H, s), 1.49 (3H, s), 1.61 (3H, s), 1.78 (3H, s), 2.33 (3H, s), 2.79 (1H, dd, J=12.6, 5.2 Hz), 2.86-2.93 (2H, m), 3.07-3.13 (2H, m), 3.75-3.82 (2H, m), 3.97-4.05 (1H, m), 4.13 (1H, d, J=7.1 Hz), 4.24 (1H, d, J=8.3 Hz), 4.31-4.35 (2H, m), 4.45-4.52 (2H, m), 4.83-4.90 (2H, m), 4.93 (1H, s), 5.22 (1H, d, J=6.8 Hz), 5.99 (1H, d, J=5.1 Hz), 6.06 (1H, t, J=8.5 Hz), 7.48 (2H, t, J=7.7 Hz), 7.61 (1H, t, J=7.4 Hz), 8.11-8.15 (2H, m).

FAB-MS; m/z: 843 (M+H)⁺.

### Example 39

### (1S,2S,3R,4S,5R,8R,9S,10R,13S)-4-(Acetoxy)-2-(benzoyloxy)-5,20-epoxy-1-hydroxy-9,10-[(1S)-2-(3-hydroxy-3-methylazetidino)ethylidenedioxy]tax-11-en-13-yl

### (2R,3S)-3-(tert-butoxycarbonylamino)-2-hydroxypentanoate

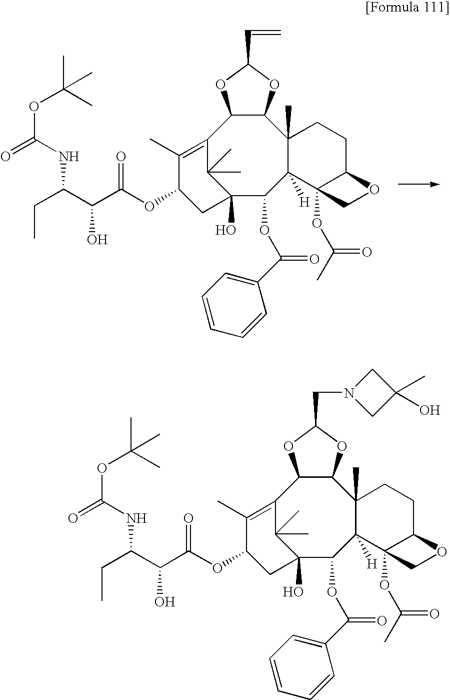

The compound obtained in Example 37, Step 2 and the compound obtained in Reference Example 3 were used as starting materials to perform the same procedure as that of Example 1 and thereby to obtain the title compound as white amorphous solid.

¹H-NMR (400 MHz, CDCl₃) δ: 1.01 (3H, t, J=7.4 Hz), 1.17-2.42 (10H, m), 1.25 (3H, s), −1.39 (9H, s), 1.49 (3H, s), 1.51 (3H, s), 1.60 (3H, s), 1.78 (3H, s), 2.33 (3H, s), 2.80 (1H, dd, J=12.5, 5.9 Hz), 2.90-2.95 (2H, m), 3.25 (2H, t, J=8.5 Hz), 3.45 (2H, t, J=8.3 Hz), 3.98-4.05 (1H, m), 4.09-4.15 (2H, m), 4.24 (1H, d, J=8.3 Hz), 4.31-4.35 (2H, m), 4.83-4.95 (3H, m), 5.22 (1H, d, J=6.8 Hz), 5.99 (1H, d, J=5.1 Hz), 6.06 (1H, t, J=8.3 Hz), 7.48 (2H, t, J=7.7 Hz), 7.61 (1H, t, J=7.4 Hz), 8.11-8.15 (2H, m).

FAB-MS; m/z: 857 (M+H)⁺.

### Example 40

### (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3R)-3-(tert-butoxycarbonylamino)-2-hydroxy-3-(2-thiazolyl)propionate

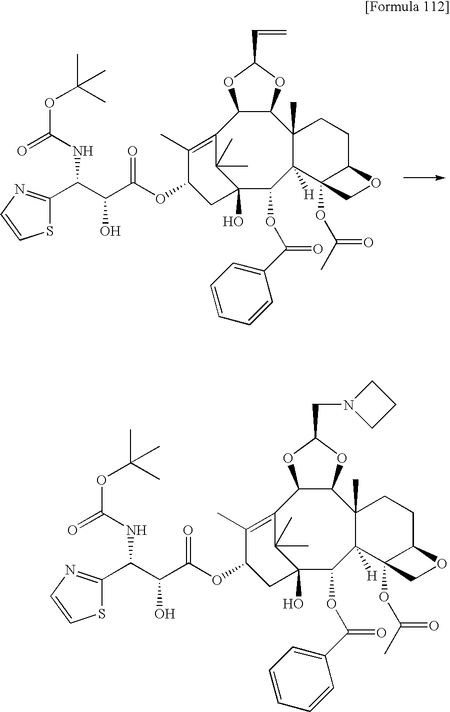

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3R)-3-(tert-butoxycarbonylamino)-2-hydroxy-3-(2-thiazolyl)propionate (Bioorg. Med. Chem. Lett., 2004, 14, 3209) was used as a starting material to perform the same procedure as that of Example 1 and thereby to obtain the title compound as white amorphous solid.

¹H-NMR (400 MHz, CDCl₃) δ: 1.24-2.16 (9H, m), 1.24 (3H, s), 1.45 (9H, s), 1.49 (3H, s), 1.61 (3H, s), 1.73 (3H, s), 2.30-2.36 (1H, m), 2.35 (3H, s), 2.74 (1H, dd, J=12.5, 5.9 Hz), 2.82 (1H, dd, J=12.5, 4.4 Hz), 2.92 (1H, d, J=5.1 Hz), 3.31-3.40 (4H, m), 4.11 (1H, d, J=7.3 Hz), 4.22 (1H, d, J=8.3 Hz), 4.33 (1H, d, J=8.3 Hz), 4.87 (1H, dd, J=5.9, 4.4 Hz), 4.92 (1H, s), 5.00 (1H, d, J=2.2 Hz), 5.21 (1H, d, J=7.1 Hz), 5.60 (1H, d, J=9.8 Hz), 5.95 (1H, d, J=9.8 Hz), 5.99 (1H, d, J=5.1 Hz), 6.12 (1H, t, J=8.2 Hz), 7.31 (1H, d, J=3.2 Hz), 7.47 (2H, t, J=7.5 Hz), 7.60 (1H, t, J=7.5 Hz), 7.76 (1H, d, J=3.2 Hz), 8.12-8.14 (2H, m).

FAB-MS; m/z: 882 (M+H)⁺.

### Example 41

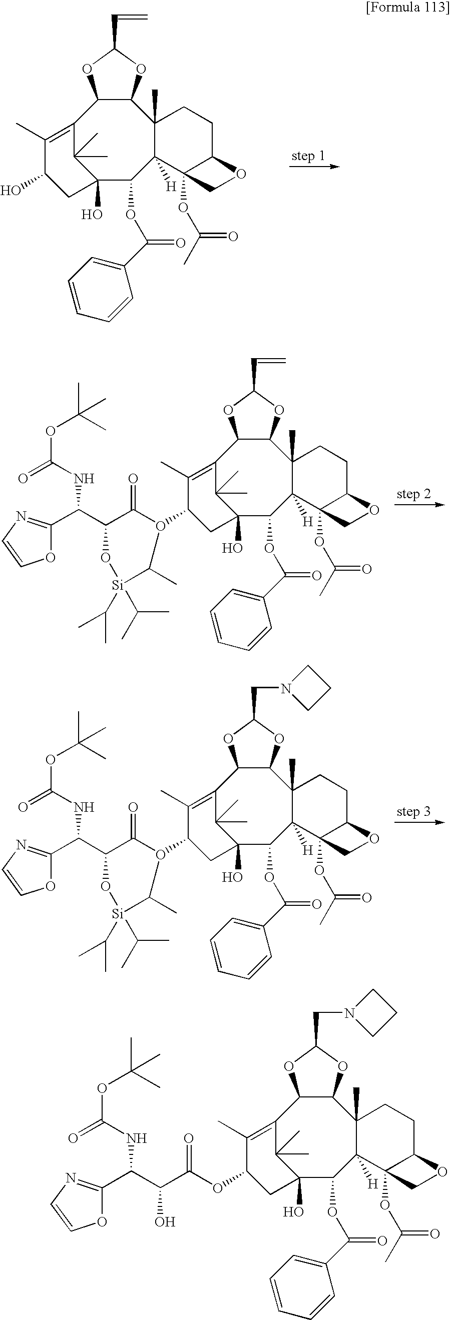

### Step 1: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3R)-3-(tert-butoxycarbonylamino)-3-(2-oxazolyl)-2-[(triisopropylsilyl)oxy]propionate

(1S,2S,3R,4S,5R,8R,9S,10R,13S)-4-(Acetoxy)-2-(benzoyloxy)-1,13-dihydroxy-5, 20-epoxy-9,10-[(1S)-2-propenylidenedioxy]tax-11-ene and the compound obtained in Reference Example 20, Step 4 were used as starting materials to perform the same procedure as that of Example 31, Step 1 and thereby to obtain the title compound as white amorphous solid.

¹H-NMR (400 MHz, CDCl₃) δ: 0.91-1.06 (21H, m), 1.31 (3H, s), 1.35-1.96 (5H, m), 1.40 (9H, s), 1.52 (3H, s), 1.65 (3H, s), 1.81 (3H, s), 2.02-2.39 (2H, m), 2.47 (3H, s), 2.97 (1H, d, J=5.9 Hz), 4.21 (2H, d, J=7.6 Hz), 4.34 (1H, d, J=8.5 Hz), 4.95 (1H, brs), 5.09 (1H, d, J=1.7 Hz), 5.21 (1H, d, J=6.6 Hz), 5.28 (1H, d, J=7.1 Hz), 5.46 (1H, d, J=10.3 Hz), 5.47 (1H, s), 5.53 (1H, s), 5.58 (1H, d, J=17.8 Hz), 5.96-6.05 (2H, m), 6.08 (1H, t, J=8.3 Hz), 7.11 (1H, s), 7.47 (2H, t, J=7.3 Hz), 7.59 (1H, t, J=7.8 Hz), 7.65 (1H, s), 8.15 (1H, d, J=7.8 Hz).

### Step 2: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3R)-3-(tert-butoxycarbonylamino)-3-(2-oxazolyl)-2-[(triisopropylsilyl)oxy]propionate

The compound obtained in Step 1 mentioned above was used as a starting material to perform the same procedure as that of Example 1 and thereby to obtain the title compound as white amorphous solid.

¹H-NMR (400 MHz, CDCl₃) δ: 0.91-1.06 (21H, m), 1.29 (3H, s), 1.40 (9H, s), 1.51 (3H, s), 1.58-2.19 (7H, m), 1.62 (3H, s), 1.78 (3H, s), 2.24-2.37 (2H, m), 2.46 (3H, s), 2.74 (1H, dd, J=12.5, 5.9 Hz), 2.81 (1H, dd, J=12.5, 4.4 Hz), 2.95 (1H, d, J=5.4 Hz), 3.31-3.40 (4H, m), 4.14 (1H, d, J=7.1 Hz), 4.21 (1H, d, J=8.5 Hz), 4.34 (1H, d, J=8.5 Hz), 4.84 (1H, t, J=5.4 Hz), 4.95 (1H, s), 5.09 (1H, s), 5.19 (1H, d, J=7.3 Hz), 5.45 (1H, d, J=10.3 Hz), 5.54 (1H, d, J=10.5 Hz), 5.98 (1H, d, J=4.9 Hz), 6.07 (1H, t, J=9.0 Hz), 7.11 (1H, s), 7.47 (2H, t, J=7.8 Hz), 7.59 (1H, t, J=7.8 Hz), 7.65 (1H, s), 8.15 (2H, d, J=8.1 Hz).

### Step 3: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3R)-3-(tert-butoxycarbonylamino)-2-hydroxy-3-(2-oxazolyl)propionate

The compound obtained in Step 2 mentioned above was used as a starting material to perform the same procedure as that of Example 31, Step 2 and thereby to obtain the title compound as white amorphous solid.

¹H-NMR (400 MHz, CDCl₃) δ: 1.25 (3H, s), 1.43 (9H, s), 1.49 (3H, s), 1.61 (3H, s), 1.79 (3H, s), 1.82-2.21 (8H, m), 2.26-2.43 (2H, m), 2.35 (3H, s), 2.75 (1H, dd, J=12.2, 5.6 Hz), 2.83 (1H, dd, J=12.5, 4.4 Hz), 2.94 (1H, d, J=4.6 Hz), 3.32-3.41 (4H, m), 4.12 (1H, d, J=7.1 Hz), 4.24 (1H, d, J=8.5 Hz), 4.33 (1H, d, J=8.3 Hz), 4.84 (1H, d, J=2.0 Hz), 4.88 (1H, t, J=4.9 Hz), 4.92 (1H, br s), 5.22 (1H, d, J=7.1 Hz), 5.50 (1H, d, J=9.0 Hz), 5.71 (1H, d, J=9.5 Hz), 6.00 (1H, d, J=4.9 Hz), 6.13 (1H, t, J=7.8 Hz), 7.11 (1H, s), 7.48 (2H, t, J=7.7 Hz), 7.61 (1H, t, J=7.6 Hz), 7.67 (1H, s), 8.13 (2H, d, J=8.1 Hz).

FAB-MS; m/z: 866 (M+H)⁺.

### Example 42

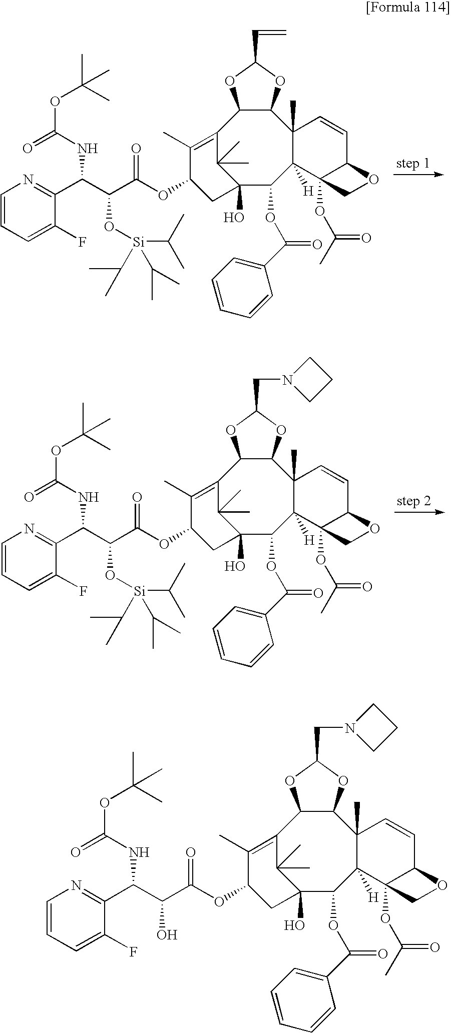

### Step 1: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-6,11-dien-13-yl

### (2R,3S)-3-(tert-butoxycarbonylamino)-3-(3-fluoro-2-pyridyl)-2-[(triisopropylsilyl)oxy]propionate

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-6,11-dien-13-yl (2R,3S)-3-(tert-butoxycarbonylamino)-3-(3-fluoro-2-pyridyl)-2-[(triisopropylsilyl)oxy]propionate (WO01/027115) was used as a starting material to perform the same procedure as that of Example 1 and thereby to obtain the title compound as white amorphous solid.

¹H-NMR (400 MHz, CDCl₃) δ: 0.85-0.92 (21H, m), 1.26-2.17 (3H, m), 1.31 (3H, s), 1.38 (9H, s), 1.55 (3H, s), 1.56 (3H, s), 1.74 (3H, s), 2.39-2.48 (2H, m), 2.51 (3H, s), 2.77 (1H, dd, J=12.5, 5.4 Hz), 2.84 (1H, dd, J=12.5, 4.6 Hz), 3.13 (1H, d, J=5.9 Hz), 3.37 (4H, t, J=7.1 Hz), 4.00 (1H, d, J=7.8 Hz), 4.32 (2H, s), 4.83 (1H, t, J=5.0 Hz), 4.90 (1H, d, J=4.4 Hz), 4.94 (1H, s), 5.11 (1H, d, J=7.8 Hz), 5.62 (1H, d, J=10.0 Hz), 5.68 (1H, dd, J=10.5, 4.4 Hz), 5.95 (1H, d, J=5.9 Hz), 6.08-6.11 (3H, m), 7.26-7.29 (1H, m), 7.41 (1H, t, J=9.3 Hz), 7.49 (2H, t, J=7.6 Hz), 7.57-7.61 (1H, m), 8.17-8.19 (2H, m), 8.40 (1H, d, J=4.6 Hz).

FAB-MS; m/z: 1048 (M+H)⁺.

### Step 2: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-6,11-dien-13-yl

### (2R,3S)-3-(tert-butoxycarbonylamino)-3-(3-fluoro-2-pyridyl)-2-hydroxypropionate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.26-1.85 (2H, m), 1.27 (3H, s), 1.39 (9H, s), 1.53 (3H, s), 1.57 (3H, s), 1.72 (3H, s), 2.10-2.17 (2H, m), 2.28-2.50 (2H, m), 2.41 (3H, s), 2.78 (1H, dd, J=12.3, 5.5 Hz), 2.85 (1H, dd, J=12.5, 4.6 Hz), 3.10 (1H, d, J=5.9 Hz), 3.38 (4H, t, J=7.1 Hz), 3.97 (1H, d, J=7.8 Hz), 4.27 (1H, d, J=8.3 Hz), 4.33 (1H, d, J=8.3 Hz), 4.66 (1H, d, J=2.4 Hz), 4.85-4.88 (2H, m), 5.13 (1H, d, J=7.6 Hz), 5.63 (1H, d, J=8.8 Hz), 5.68 (1H, dd, J=10.3, 4.2 Hz), 5.96 (1H, d, J=5.9 Hz), 6.07-6.12 (2H, m), 6.21 (1H, d, J=8.3 Hz), 7.29-7.33 (1H, m), 7.43-7.51 (3H, m), 7.60 (1H, t, J=7.4 Hz), 8.14-8.17 (2H, m), 8.40 (1H, d, J=4.6 Hz).

FAB-MS; m/z: 892 (M+H)⁺.

### Example 43

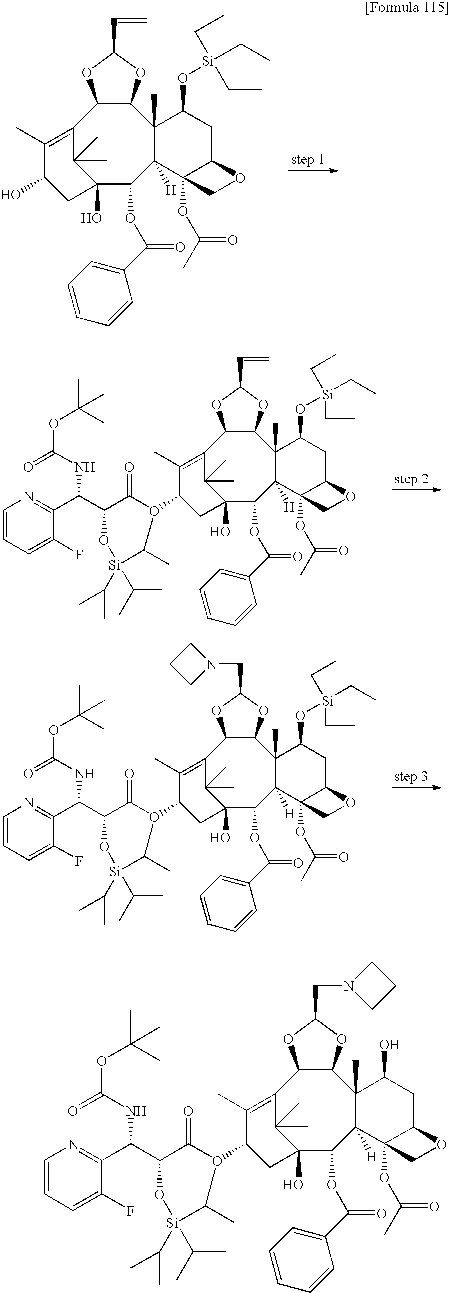

### Step 1: (1S,2S,3R,4S,5R,7S,8R,9S,10R,13S)-4-(Acetoxy)-2-(benzoyloxy)-5,20-epoxy-1-hydroxy-9,10-[(1S)-2-propenylidenedioxy]-7-[(triethylsilyl)oxy]tax-11-en-13-yl

### (2R,3S)-3-(tert-butoxycarbonylamino)-3-(3-fluoro-2-pyridyl)-2-[(triisopropylsilyl)oxy]propionate

(1S,2S,3R,4S,5R,7S,8R,9S,10R,13S)-4-(Acetoxy)-2-(benzoyloxy)-1,13-dihydroxy-5,20-epoxy-9,10-[(1S)-2-propenylidenedioxy]-7-[(triethylsilyl)oxy]tax-11-ene (Bioorg. Med. Chem. Lett., 2002, 12, 1083) and (3R,4S)-1-(tert-butoxycarbonyl)-4-(3-fluoro-2-pyridyl)-3-[(triisopropylsilyl)oxy]-2-azetidinone (Bioorg. Med. Chem. Lett., 2004, 14, 3209) were used as starting materials to perform the same procedure as that of Example 31, Step 1 and thereby to obtain the title compound as white amorphous solid.

¹H-NMR (400 MHz, CDCl₃) δ: 0.60-0.68 (6H, m), 0.86-0.98 (21H, m), 0.99 (9H, t, J=7.8 Hz), 1.26 (3H, s), 1.37 (9H, s), 1.56 (3H, s), 1.57 (3H, s), 1.77 (1H, s), 1.78 (3H, s), 2.04-2.16 (2H, m), 2.30-2.44 (2H, m), 2.48 (3H, s), 3.24 (1H, d, J=5.6 Hz), 3.94 (1H, dd, J=10.7, 5.4 Hz), 4.35 (1H, d, J=8.3 Hz), 4.43 (1H, d, J=8.1 Hz), 4.60 (1H, d, J=8.8 Hz), 4.85 (1H, dd, J=9.0, 4.9 Hz), 4.94 (1H, d, J=2.0 Hz), 5.06 (1H, d, J=5.9 Hz), 5.29 (1H, d, J=9.0 Hz), 5.45 (1H, d, J=10.5 Hz), 5.56 (1H, d, J=17.6 Hz), 5.61 (1H, d, J=10.3 Hz), 5.92 (1H, d, J=5.6 Hz), 6.04 (1H, d, J=9.8 Hz), 6.07-6.18 (2H, m), 7.25-7.30 (1H, m), 7.40 (1H, t, J=9.8 Hz), 7.47 (2H, t, J=7.6 Hz), 7.57 (1H, t, J=7.3 Hz), 8.12 (2H, d, J=7.1 Hz), 8.39 (1H, d, J=4.6 Hz).

FAB-MS; m/z: 1137 (M+H)⁺.

### Step 2: (1S,2S,3R,4S,5R,7S,8R,9S,10R,13S)-4-(Acetoxy)-9,10-[(1S)-2-(azetidino)ethylidenedioxy]-2-(benzoyloxy)-5,20-epoxy-1-hydroxy-7-[(triethylsilyl)oxy]tax-11-en-13-yl

### (2R,3S)-3-(tert-butoxycarbonylamino)-3-(3-fluoro-2-pyridyl)-2-[(triisopropylsilyl)oxy]propionate

The compound obtained in Step 1 mentioned above was used as a starting material to perform the same procedure as that of Example 1 and thereby to obtain the title compound as white amorphous solid.

¹H-NMR (400 MHz, CDCl₃) δ: 0.63 (6H, q, J=8.5 Hz), 0.88-0.93 (21H, m), 0.98 (9H, t, J=7.8 Hz), 1.25 (3H, s), 1.37 (9H, s), 1.51 (3H, s), 1.54 (3H, s), 1.76 (3H, s), 1.77 (1H, s), 2.03-2.19 (4H, m), 2.28-2.40 (2H, m), 2.48 (3H, s), 2.85 (2H, d, J=4.9 Hz), 3.22 (1H, d, J=5.6 Hz), 3.33 (4H, t, J=7.0 Hz), 3.91 (1H, dd, J=10.3, 4.9 Hz), 4.34 (1H, d, J=8.3 Hz), 4.45 (2H, d, J=7.8 Hz), 4.68 (1H, t, J=5.1 Hz), 4.84 (1H, dd, J=9.0, 5.1 Hz), 4.93 (1H, d, J=2.0 Hz), 5.19 (1H, d, J=8.8 Hz), 5.61 (1H, d, J=10.3 Hz), 5.89 (1H, d, J=5.6 Hz), 6.05 (1H, d, J=10.0 Hz), 6.11 (1H, t, J=9.3 Hz), 7.25-7.30 (1H, m), 7.40 (1H, t, J=8.3 Hz), 7.47 (2H, t, J=7.7 Hz), 7.57 (1H, t, J=7.6 Hz), 8.12 (2H, d, J=7.1 Hz), 8.40 (1H, d, J=4.4 Hz).

FAB-MS; m/z: 1180 (M+H)⁺.

### Step 3: (1S,2S,3R,4S,5R,7S,8S,9S,10R,13S)-4-(Acetoxy)-9,10-[(1S)-2-(azetidino)ethylidenedioxy]-2-(benzoyloxy)-1,7-dihydroxy-5,20-epoxytax-11-en-13-yl

### (2R,3S)-3-(tert-butoxycarbonylamino)-3-(3-fluoro-2-pyridyl)-2-hydroxypropionate

The compound obtained in Step 2 mentioned above (0.55 g) was dissolved in pyridine (2 ml), and the solution was added with hydrogen fluoride/pyridine (1 ml) at 0° C., and stirred at room temperature for 22.5 hours. The reaction mixture was poured into ice water, neutralized with sodium hydrogencarbonate, and then extracted with ethyl acetate. The organic layer was dried, and then the solvent was evaporated under reduced pressure. The resulting residue was purified by silica gel chromatography [developing solvent; chloroform:methanol=50:1 (v/v)] to obtain the title compound (0.24 g) as white amorphous solid.

¹H-NMR (400 MHz, CDCl₃) δ: 1.30 (3H, s), 1.40 (9H, s), 1.63 (3H, s), 1.67 (3H, s), 1.74 (3H, s), 1.90 (1H, s), 2.06-2.28 (6H, m), 2.38 (3H, s), 2.40-2.50 (1H, m), 2.77 (1H, dd, J=12.7, 5.6 Hz), 2.83 (1H, dd, J=12.7, 4.4 Hz), 2.94 (1H, d, J=4.9 Hz), 3.31-3.41 (4H, m), 3.83 (1H, d, J=7.3 Hz), 4.07 (1H, br s), 4.32 (1H, d, J=9.0 Hz), 4.39 (1H, d, J=8.5 Hz), 4.61-4.72 (2H, m), 4.85 (1H, t, J=4.6 Hz), 5.10 (1H, s), 5.18 (1H, d, J=7.3 Hz), 5.62 (1H, d, J=8.5 Hz), 6.04 (1H, d, J=4.4 Hz), 6.10 (1H, t, J=8.5 Hz), 6.21 (1H, d, J=8.5 Hz), 7.29-7.34 (1H, m), 7.42-7.51 (3H, m), 7.60 (1H, t, J=7.6 Hz), 8.13 (2H, d, J=8.3 Hz), 8.40 (1H, d, J=3.4 Hz).

FAB-MS; m/z: 910 (M+H)⁺.

### Example 44

### (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-11-en-13-yl

### (2R,3R)-3-(tert-butoxycarbonylamino)-4,4-difluoro-2-hydroxybutyrate

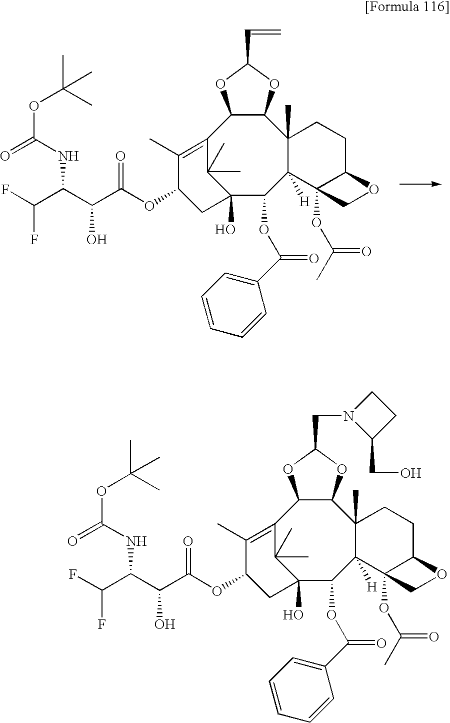

The compound obtained in Example 31, Step 2 and the compound obtained in Reference Example 25 were used as starting materials to perform the same procedure as that of Example 1 and thereby to obtain the title compound as white amorphous solid.

¹H-NMR (400 MHz, CDCl₃) δ: 1.22-2.05 (8H, m), 1.23 (3H, s), 1.42 (9H, s), 1.49 (3H, s), 1.58 (3H, s), 1.77 (3H, s), 2.21-2.38 (3H, m), 2.34 (3H, s), 2.84 (1H, dd, J=12.7, 4.6 Hz), 2.89-2.94 (2H, m), 3.12 (1H, q, J=8.2 Hz), 3.44 (1H, dd, J=11.5, 2.7 Hz), 3.48-3.54 (2H, m), 3.66 (1H, dd, J=11.5, 2.9 Hz), 4.13 (1H, d, J=7.1 Hz), 4.24 (1H, d, J=8.3 Hz), 4.33 (1H, d, J=8.3 Hz), 4.48-4.58 (1H, m), 4.64 (1H, s), 4.90 (1H, t, J=4.9 Hz), 4.93 (1H, s), 5.23-5.27 (2H, m), 5.89 (1H, td, J=55.7, 5.4 Hz), 5.99 (1H, d, J=5.4 Hz), 6.10 (1H, t, J=8.3 Hz), 7.48 (2H, t, J=7.7 Hz), 7.59-7.64 (1H, m), 8.11-8.14 (2H, m).

FAB-MS; m/z: 879 (M+H)⁺.

### Example 45

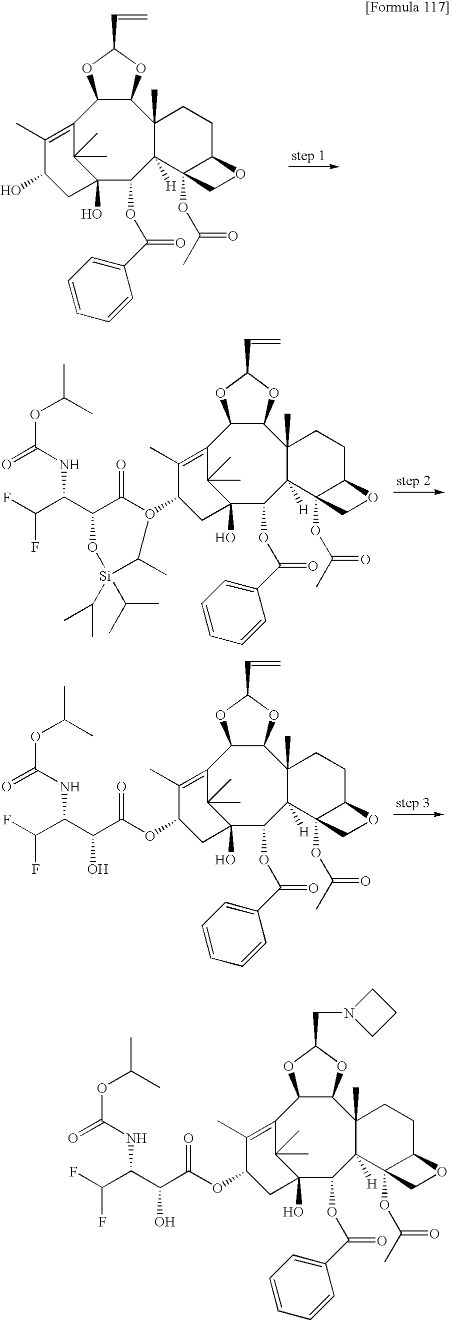

### Step 1: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3R)-4,4-difluoro-3-(isopropoxycarbonylamino)-2-[(triisopropylsilyl)oxy]butyrate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-11-ene and the compound obtained in Reference Example 21 were used as starting materials to perform the same procedure as that of Example 31, Step 1 and thereby to obtain the title compound as white amorphous solid.

¹H-NMR (400 MHz, CDCl₃) δ: 1.08-2.10 (32H, m), 1.30 (3H, s), 1.51 (3H, s), 1.64 (3H, s), 1.78 (3H, s), 2.23 (2H, d, J=8.8 Hz), 2.36 (3H, s), 2.93 (1H, d, J=5.1 Hz), 4.20 (1H, d, J=7.1 Hz), 4.21 (1H, d, J=8.3 Hz), 4.33-4.43 (2H, m), 4.79-4.92 (3H, m), 5.10 (1H, d, J=10.5 Hz), 5.20 (1H, d, J=6.1 Hz), 5.26 (1H, d, J=6.8 Hz), 5.45 (1H, d, J=10.5 Hz), 5.57 (1H, d, J=17.3 Hz), 5.66-6.07 (4H, m), 7.50 (2H, t, J=7.7 Hz), 7.59-7.63 (1H, m), 8.14-8.16 (2H, m).

FAB-MS; m/z: 948 (M+H)⁺.

### Step 2: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3R)-4,4-difluoro-2-hydroxy-3-(isopropoxycarbonylamino)butyrate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.19-1.25 (6H, m), 1.25 (3H, s), 1.47-2.11 (6H, m), 1.49 (3H, s), 1.64 (3H, s), 1.77 (3H, s), 2.32-2.38 (1H, m), 2.34 (3H, s), 2.93 (1H, d, J=4.9 Hz), 4.17 (1H, d, J=7.1 Hz), 4.26 (1H, d, J=8.5 Hz), 4.34 (1H, d, J=8.5 Hz), 4.60-4.66 (2H, m), 4.85-4.94 (3H, m), 5.25 (1H, d, J=6.3 Hz), 5.30-5.34 (2H, m), 5.47 (1H, d, J=10.5 Hz), 5.58 (1H, d, J=17.3 Hz), 5.77-6.05 (3H, m), 6.15 (1H, t, J=8.1 Hz), 7.48 (2H, t, J=7.7 Hz), 7.59-7.64 (1H, m), 8.12-8.14 (2H, m).

FAB-MS; m/z: 792 (M+H)⁺.

### Step 3: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3R)-4,4-difluoro-2-hydroxy-3-(isopropoxycarbonylamino)butyrate

The compound obtained in Step 2 mentioned above was used as a starting material to perform the same procedure as that of Example 1 and thereby to obtain the title compound as white amorphous solid.

¹H-NMR (400 MHz, CDCl₃) δ: 1.21 (6H, dd, J=16.8, 6.3 Hz), 1.23 (3H, s), 1.47-2.16 (9H, m), 1.49 (3H, s), 1.61 (3H, s), 1.75 (3H, s), 2.30-2.36 (1H, m), 2.33 (3H, s), 2.74 (1H, dd, J=12.6, 5.6 Hz), 2.83 (1H, dd, J=12.6, 4.4 Hz), 2.91 (1H, d, J=4.9 Hz), 3.32-3.40 (4H, m), 4.10 (1H, d, J=7.1 Hz), 4.25 (1H, d, J=8.3 Hz), 4.33 (1H, d, J=8.3 Hz), 4.55-4.65 (2H, m), 4.86-4.93 (3H, m), 5.21 (1H, d, J=6.8 Hz), 5.40 (1H, d, J=10.0 Hz), 5.91 (1H, td, J=55.7, 5.1 Hz), 6.01 (1H, d, J=5.1 Hz), 6.13 (1H, t, J=8.4 Hz), 7.48 (2H, t, J=7.7 Hz), 7.59-7.63 (1H, m), 8.12-8.14 (2H, m).

FAB-MS; m/z: 835 (M+H)⁺.

### Example 46

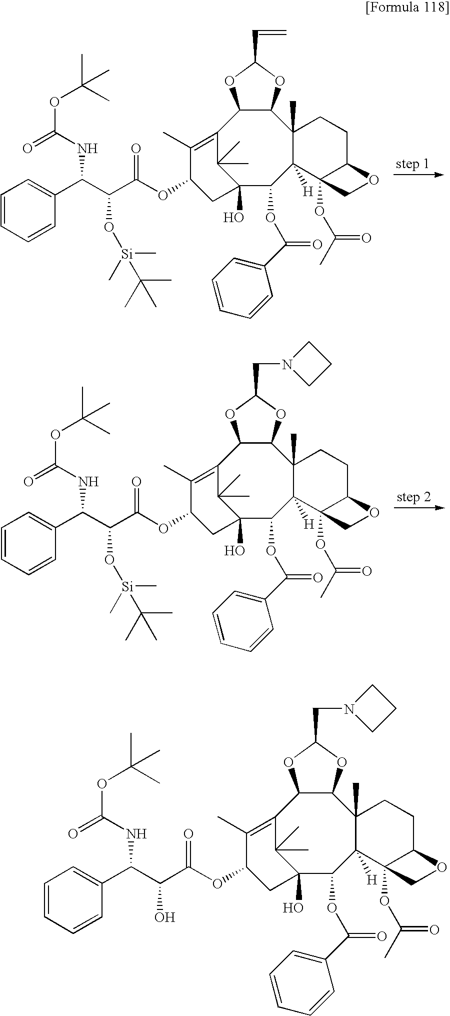

### Step 1: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3S)-3-(tert-butoxycarbonylamino)-2-[(tert-butyldimethylsilyl)oxy]-3-phenylpropionate

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

(2R,3S)-3-(tert-butoxycarbonylamino)-2-[(tert-butyldimethylsilyl)oxy]-3-phenylpropionate (WO96/33998) was used as a starting material to perform the same procedure as that of Example 1 and thereby to obtain the title compound as white amorphous solid.

¹H-NMR (400 MHz, CDCl₃) δ: −0.34 (3H, s), −0.11 (3H, s), 0.74 (9H, s), 1.33 (3H, s), 1.39 (9H, s), 1.46-2.33 (9H, m), 1.52 (3H, s), 1.62 (3H, s), 1.76 (3H, s), 2.51 (3H, s), 2.74 (1H, dd, J=12.2, 5.6 Hz), 2.81 (1H, dd, J=12.2, 4.6 Hz), 2.94 (1H, d, J=5.4 Hz), 3.32-3.39 (4H, m), 4.14 (1H, d, J=7.3 Hz), 4.24 (1H, d, J=8.5 Hz), 4.34 (1H, d, J=8.3 Hz), 4.5.7 (1H, s), 4.84 (1H, t, J=5.4 Hz), 4.97 (1H, brs), 5.18 (1H, d, J=7.1 Hz), 5.32 (1H, d, J=9.3 Hz), 5.48 (1H, d, J=9.0 Hz), 5.98 (1H, d, J=5.4 Hz), 6.20 (1H, t, J=8.8 Hz), 7.27-7.39 (5H, m), 7.48 (2H, t, J=7.6 Hz), 7.60 (1H, t, J=7.3 Hz), 8.15 (2H, d, J=7.6 Hz).

FAB-MS; m/z: 989 (M+H)⁺.

### Step 2: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3S)-3-(tert-butoxycarbonylamino)-2-hydroxy-3-phenylpropionate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.26 (3H, s), 1.41 (9H, s), 1.48 (3H, s), 1.60 (3H, s), 1.67 (3H, s), 1.77-2.43 (10H, m), 2.27 (3H, s), 2.73 (1H, dd, J=12.7, 5.6 Hz), 2.82 (1H, dd, J=12.9, 4.4 Hz), 2.90 (1H, d, J=5.4 Hz), 3.32-3.38 (4H, m), 4.08 (1H, d, J=7.1 Hz), 4.24 (1H, d, J=8.8 Hz), 4.31 (1H, d, J=8.3 Hz), 4.63 (1H, brs), 4.86 (1H, t, J=4.9 Hz), 4.91 (1H, s), 5.19 (1H, d, J=7.1 Hz), 5.31 (1H, d, J=9.3 Hz), 5.70 (1H, d, J=9.3 Hz), 6.00 (1H, d, J=4.9 Hz), 6.06 (1H, t, J=8.1 Hz), 7.29 (1H, t, J=6.8 Hz), 7.36 (2H, t, J=7.6 Hz), 7.42 (2H, d, J=7.3 Hz), 7.47 (2H, t, J=7.7 Hz), 7.60 (1H, t, J=7.6 Hz), 8.12 (2H, d, J=7.6 Hz).

FAB-MS; m/z: 875 (M+H)⁺.

### Example 47

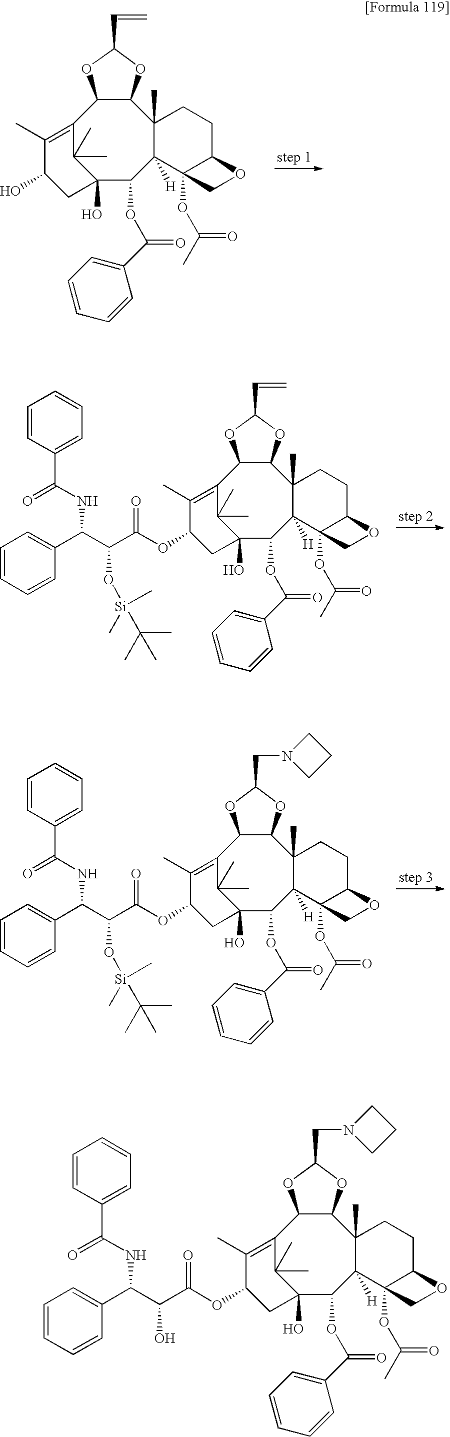

### Step 1: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3S)-3-(benzoylamino)-2-[(tert-butyldimethylsilyl)oxy]-3-phenylpropionate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-11-ene and (3R,4S)-1-benzoyl-3-[(tert-butyldimethylsilyl)oxy]-2-azetidinone (Bioorg. Med. Chem. Lett., 1993, 3, 2467) were used as starting materials to perform the same procedure as that of Example 31, Step 1 and thereby to obtain the title compound as white amorphous solid.

¹H-NMR (400 MHz, CDCl₃) δ: −0.33 (3H, s), −0.03 (3H, s), 0.80 (9H, s), 1.30 (3H, s), 1.52 (3H, s), 1.64 (3H, s), 1.76-2.33 (7H, m), 1.79 (3H, s), 2.55 (3H, s), 2.92 (1H, d, J=4.9 Hz), 4.20 (1H, d, J=6.8 Hz), 4.25 (1H, d, J=8.5 Hz), 4.35 (1H, d, J=8.8 Hz), 4.71 (1H, d, J=2.2 Hz), 4.98 (1H, br s), 5.22 (1H, d, J=5.9 Hz), 5.27 (1H, d, J=7.3 Hz), 5.46 (1H, d, J=10.5 Hz), 5.58 (1H, d, J=17.3 Hz), 5.78 (1H, d, J=9.3 Hz), 5.96-6.07 (2H, m), 6.23 (1H, t, J=8.3 Hz), 7.14 (1H, d, J=9.5 Hz), 7.24-7.56 (9H, m), 7.60 (1H, t, J=7.1 Hz), 7.78 (2H, d, J=7.8 Hz), 8.15 (2H, d, J=7.8 Hz).

### Step 2: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3S)-3-(benzoylamino)-2-[(tert-butyldimethylsilyl)oxy]-3-phenylpropionate

The compound obtained in Step 1 mentioned above was used as a starting material to perform the same procedure as that of Example 1 and thereby to obtain the title compound as white amorphous solid.

¹H-NMR (400 MHz, CDCl₃) δ: −0.34 (3H, s), −0.03 (3H, s), 0.79 (9H, s), 1.26 (3H, s), 1.28 (3H, s), 1.51 (3H, s), 1.55-2.32 (9H, m), 1.77 (3H, s), 2.54 (3H, s), 2.73 (1H, dd, J=12.5, 5.6 Hz), 2.81 (1H, dd, J=12.5, 4.2 Hz), 2.91 (1H, d, J=4.6 Hz), 3.31-3.39 (4H, m), 4.13 (1H, d, J=7.3 Hz), 4.25 (1H, d, J=8.3 Hz), 4.35 (1H, d, J=8.1 Hz), 4.71 (1H, d, J=1.7 Hz), 4.84 (1H, t, J=4.4 Hz), 4.97 (1H, br s), 5.17 (1H, d, J=7.1 Hz), 5.77 (1H, dd, J=9.0, 1.7 Hz), 5.99 (1H, d, J=5.4 Hz), 6.22 (1H, t, J=8.8 Hz), 7.13 (1H, d, J=8.8 Hz), 7.23-7.55 (9H, m), 7.60 (1H, t, J=7.6 Hz), 7.78 (2H, d, J=7.1 Hz), 8.15 (2H, d, J=7.3 Hz).

### Step 3: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3S)-3-(benzoylamino)-2-hydroxy-3-phenylpropionate

The compound obtained in Step 2 mentioned above was used as a starting material to perform the same procedure as that of Example 31, Step 2 and thereby to obtain the title compound as white amorphous solid.

¹H-NMR (400 MHz, CDCl₃) δ: 1.21 (3H, s), 1.42 (3H, s), 1.58 (3H, s), 1.65 (3H, s), 1.75-2.41 (10H, m), 2.31 (3H, s), 2.72 (1H, dd, J=12.2, 5.9 Hz), 2.80 (1H, dd, J=12.5, 4.4 Hz), 2.87 (1H, d, J=4.9 Hz), 3.30-3.38 (4H, m), 4.03 (1H, d, J=7.1 Hz), 4.24 (1H, d, J=8.8 Hz), 4.32 (1H, d, J=8.5 Hz), 4.76 (1H, d, J=2.4 Hz), 4.83 (1H, t, J=5.4 Hz), 4.92 (1H, brs), 5.11 (1H, d, J=7.3 Hz), 5.86 (1H, dd, J=9.3, 2.2 Hz), 5.99 (1H, d, J=5.1 Hz), 6.07 (1H, t, J=8.1 Hz), 7.25-7.55 (10H, m), 7.60 (1H, t, J=7.6 Hz), 7.83 (2H, d, J=8.1 Hz), 8.12 (2H, d, J=8.5 Hz).

FAB-MS; m/z: 879 (M+H)⁺.

### Example 48

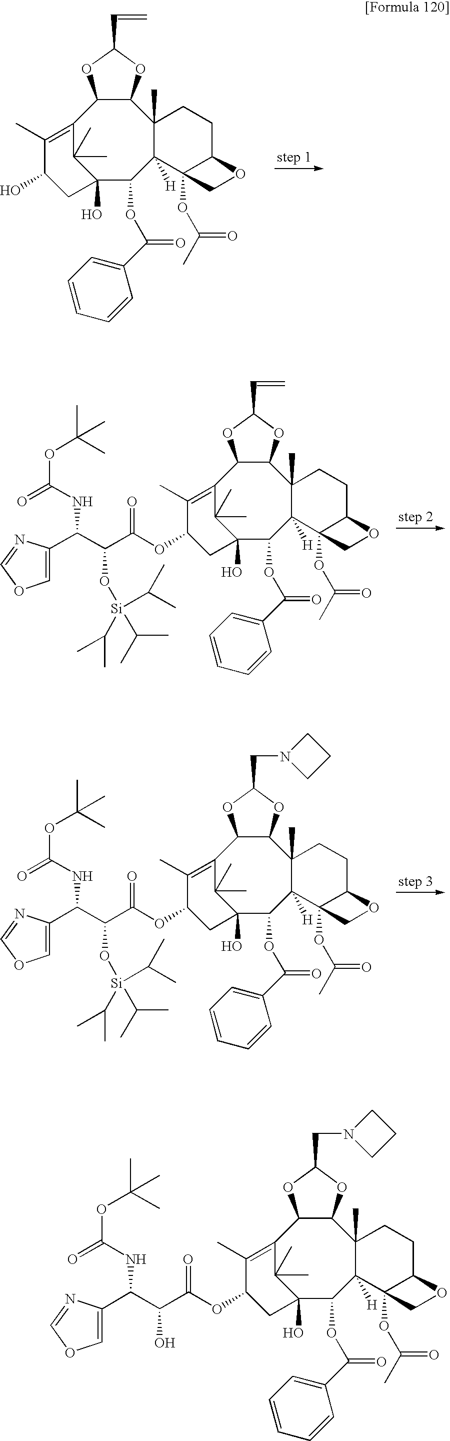

### Step 1: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3S)-3-(tert-butoxycarbonylamino)-3-(4-oxazolyl)-2-[(triisopropylsilyl)oxy]propionate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-11-ene and the compound obtained in Reference Example 22, Step 4 were used as starting materials to perform the same procedure as that of Example 31, Step 1 and thereby to obtain the title compound as white amorphous solid.

¹H-NMR (400 MHz, CDCl₃) δ: 0.93-1.03 (21H, m), 1.30 (3H, s), 1.41 (9H, s), 1.52 (3H, s), 1.52-2.36 (7H, m), 1.65 (3H, s), 1.81 (3H, s), 2.47 (3H, s), 2.96 (1H, d, J=5.4 Hz), 4.20 (1H, d, J=8.1 Hz), 4.21 (1H, d, J=7.3 Hz), 4.34 (1H, d, J=8.3 Hz), 4.96 (1H, s), 5.15 (1H, s), 5.21 (1H, d, J=6.1 Hz), 5.24-5.31 (2H, m), 5.46 (1H, d, J=10.3 Hz), 5.58 (1H, d, J=17.3 Hz), 5.95-6.05 (2H, m), 6.08 (1H, t, J=8.8 Hz), 7.46 (1H, t, J=7.8 Hz), 7.55-7.61 (2H, m), 7.85 (1H, s), 8.14 (1H, d, J=7.6 Hz).

### Step 2: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3S)-3-(tert-butoxycarbonylamino)-3-(4-oxazolyl)-2-[(triisopropylsilyl)oxy]propionate

The compound obtained in Step 1 mentioned above was used as a starting material to perform the same procedure as that of Example 1 and thereby to obtain the title compound as white amorphous solid.

¹H-NMR (400 MHz, CDCl₃) δ: 0.92-1.03 (21H, m), 1.28 (3H, s), 1.40 (9H, s), 1.51 (3H, s), 1.55-2.33 (9H, m), 1.61 (3H, s), 1.79 (3H, s), 2.47 (3H, s), 2.73 (1H, dd, J=12.5, 5.6 Hz), 2.81 (1H, dd, J=12.2, 4.2 Hz), 2.95 (1H, d, J=5.4 Hz), 3.31-3.38 (4H, m), 4.14 (1H, d, J=7.6 Hz), 4.20 (1H, d, J=8.1 Hz), 4.34 (1H, d, J=8.3 Hz), 4.83 (1H, t, J=5.1 Hz), 4.95 (1H, s), 5.14 (1H, s), 5.19 (1H, d, J=7.8 Hz), 5.23 (1H, d, J=10.3 Hz), 5.27 (1H, d, J=10.5 Hz), 5.97 (1H, d, J=5.4 Hz), 6.07 (1H, t, J=9.0 Hz), 7.46 (2H, t, J=7.6 Hz), 7.55-7.61 (2H, m), 7.85 (1H, s), 8.14 (2H, d, J=8.1 Hz).

### Step 3: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3S)-3-(tert-butoxycarbonylamino)-2-hydroxy-3-(4-oxazolyl)propionate

The compound obtained in Step 2 mentioned above was used as a starting material to perform the same procedure as that of Example 31, Step 2 and thereby to obtain the title compound as white amorphous solid.

¹H-NMR (400 MHz, CDCl₃) δ: 1.25 (3H, s), 1.42 (9H, s), 1.49 (3H, s), 1.61 (3H, s), 1.73 (3H, s), 1.70-2.47 (10H, m), 2.34 (3H, s), 2.74 (1H, dd, J=12.2, 5.9 Hz), 2.82 (1H, dd, J=12.5, 4.6 Hz), 2.92 (1H, d, J=5.1 Hz), 3.30-3.40 (4H, m), 4.11 (1H, d, J=7.1 Hz), 4.23 (1H, d, J=8.3 Hz), 4.32 (1H, d, J=8.3 Hz), 4.80 (1H, d, J=2.2 Hz), 4.86 (1H, t, J=4.6 Hz), 4.92 (1H, brs), 5.21 (1H, d, J=7.3 Hz), 5.32 (1H, d, J=9.8 Hz), 5.55 (1H, d, J=9.5 Hz), 6.00 (1H, d, J=4.9 Hz), 6.10 (1H, t, J=8.1 Hz), 7.47 (2H, t, J=7.7 Hz), 7.60 (1H, t, J=7.6 Hz), 7.67 (1H, s), 7.87 (1H, s), 8.13 (2H, d, J=8.3 Hz).

FAB-MS; m/z: 866 (M+H)⁺.

### Example 49

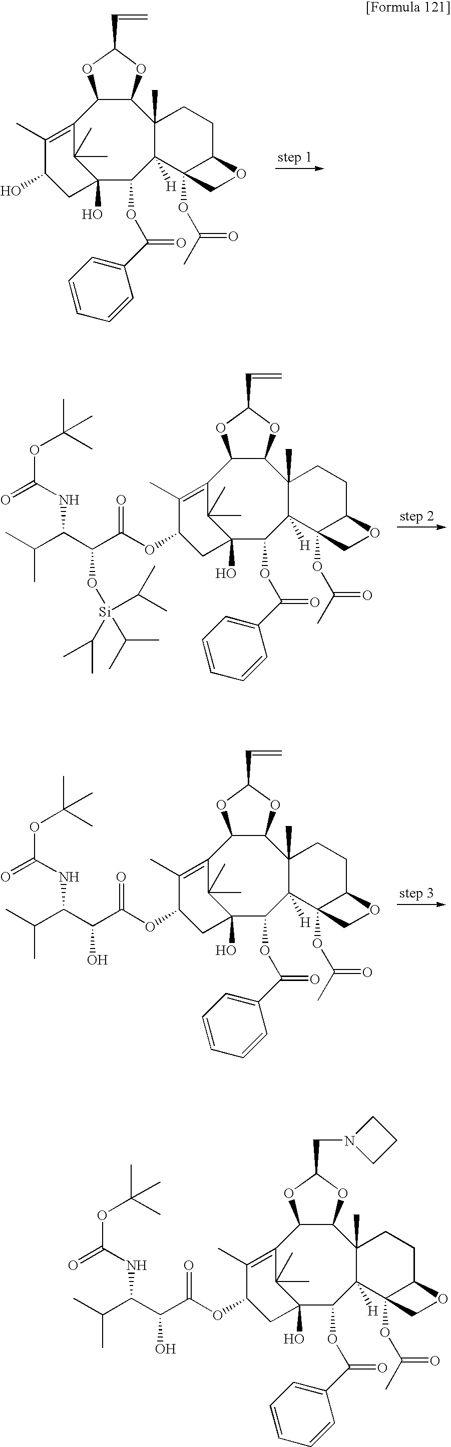

### Step 1: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3S)-3-(tert-butoxycarbonylamino)-4-methyl-2-[(triisopropylsilyl)oxy]pentanoate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-11-ene and the compound obtained in Reference Example 23, Step 3 were used as starting materials to perform the same procedure as that of Example 31, Step 1 and thereby to obtain the title compound as white amorphous solid.

¹H-NMR (400 MHz, CDCl₃) δ: 1.01-2.05 (33H, m), 1.30 (3H, s), 1.36 (9H, s), 1.52 (3H, s), 1.64 (3H, s), 1.79 (3H, s), 2.20-2.33 (2H, m), 2.36 (3H, s), 2.92 (1H, d, J=5.1 Hz), 3.64 (1H, t, J=10.3 Hz), 4.19-4.22 (2H, m), 4.34 (1H, d, J=8.5 Hz), 4.72 (1H, s), 4.78 (1H, d, J=10.3 Hz), 4.93 (1H, s), 5.20 (1H, d, J=6.1 Hz), 5.26 (1H, d, J=7.1 Hz), 5.45 (1H, d, J=10.3 Hz), 5.57 (1H, d, J=17.3 Hz), 5.95-6.04 (3H, m), 7.49 (2H, t, J=7.6 Hz), 7.61 (1H, t, J=7.6 Hz), 8.15 (2H, d, J=7.6 Hz).

FAB-MS; m/z: 954 (M+H)⁺, 976 (M+Na)⁺.

### Step 2: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3S)-3-(tert-butoxycarbonylamino)-2-hydroxy-4-methylpentanoate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.02 (3H, d, J=6.8 Hz), 1.06 (3H, d, J=6.8 Hz), 1.24-2.09 (7H, m), 1.26 (3H, s), 1.38 (9H, s), 1.50 (3H, s), 1.64 (3H, s), 1.81 (3H, s), 2.31-2.41 (1H, m), 2.35 (3H, s), 2.93 (1H, d, J=5.1 Hz), 3.80 (1H, t, J=9.6 Hz), 3.98 (1H, br s), 4.19 (1H, d, J=7.1 Hz), 4.24 (1H, d, J=8.5 Hz), 4.33 (1H, d, J=8.5 Hz), 4.50 (1H, s), 4.88 (1H, d, J=10.3 Hz), 4.94 (1H, s), 5.25 (1H, d, J=6.3 Hz), 5.31 (1H, d, J=6.8 Hz), 5.46 (1H, d, J=10.7 Hz), 5.58 (1H, d, J=17.3 Hz), 5.97-6.08 (3H, m), 7.48 (2H, t, J=7.6 Hz), 7.61 (1H, t, J=7.6 Hz), 8.13 (2H, d, J=7.6 Hz).

FAB-MS; m/z: 798 (M+H)⁺, 820 (M+Na)⁺.

### Step 3: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3S)-3-(tert-butoxycarbonylamino)-2-hydroxy-4-methylpentanoate

The compound obtained in Step 2 mentioned above was used as a starting material to perform the same procedure as that of Example 1 and thereby to obtain the title compound as white amorphous solid.

¹H-NMR (400 MHz, CDCl₃) δ: 1.02 (3H, d, J=6.6 Hz), 1.06 (3H, d, J=6.8 Hz), 1.24-2.40 (11H, m), 1.25 (3H, s), 1.38 (9H, s), 1.50 (3H, s), 1.61 (3H, s), 1.79 (3H, s), 2.34 (3H, s), 2.74 (1H, dd, J=12.6, 5.9 Hz), 2.82 (1H, dd, J=12.6, 4.3 Hz), 2.92 (1H, d, J=5.1 Hz), 3.31-3.40 (4H, m), 3.79 (1H, t, J=9.6 Hz), 4.12 (1H, d, J=7.1 Hz), 4.24 (1H, d, J=8.5 Hz), 4.33 (1H, d, J=8.5 Hz), 4.50 (1H, d, J=1.2 Hz), 4.85-4.88 (2H, m), 4.93 (1H, s), 5.22 (1H, d, J=6.8 Hz), 5.99-6.06 (2H, m), 7.48 (2H, t, J=7.7 Hz), 7.59-7.63 (1H, m), 8.12-8.14 (2H, m).

FAB-MS; m/z: 841 (M+H)⁺.

### Example 50

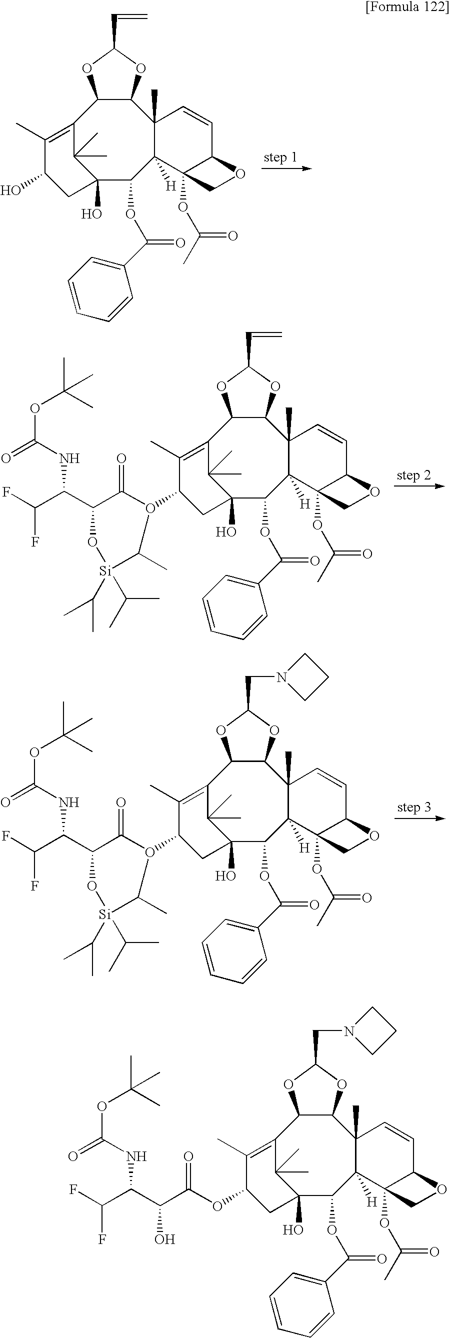

### Step 1: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-6,11-dien-13-yl

### (2R,3R)-3-(tert-butoxycarbonylamino)-4,4-difluoro-2-[(triisopropylsilyl)oxy]butyrate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-6,11-dien-13-yl (WO01/027115) and (3R,4R)-1-(tert-butoxycarbonylamino)-4-(difluoromethyl)-3-[(triisopropylsilyl)oxy]-2-azetidinone were used as starting materials to perform the same procedure as that of Example 31, Step 1 and thereby to obtain the title compound as white amorphous solid.

¹H-NMR (400 MHz, CDCl₃) δ: 1.02-1.60 (27H, m), 1.26 (3H, s), 1.38 (3H, s), 1.54 (3H, s), 1.58 (3H, s), 1.72 (3H, s), 1.84 (1H, s), 2.28-2.36 (2H, m), 2.39 (3H, s), 3.09 (1H, d, J=5.9 Hz), 4.03 (1H, d, J=7.8 Hz), 4.27-4.44 (3H, m), 4.86 (2H, d, J=4.2 Hz), 5.04 (1H, d, J=10.3 Hz), 5.19 (1H, d, J=7.8 Hz), 5.21 (1H, d, J=6.3 Hz), 5.48 (1H, d, J=10.5 Hz), 5.60 (1H, d, J=17.3 Hz), 5.64-5.71 (1H, m), 5.79 (1H, d, J=6.3 Hz), 5.91-6.07 (3H, m), 6.11 (1H, d, J=10.3 Hz), 7.52 (2H, t, J=7.4 Hz), 7.62 (1H, t, J=7.4 Hz), 8.17 (2H, d, J=7.3 Hz).

### Step 2: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-6,11-dien-13-yl

### (2R,3R)-3-(tert-butoxycarbonylamino)-4,4-difluoro-2-[(triisopropylsilyl)oxy]butyrate

The compound obtained in Step 1 mentioned above was used as a starting material to perform the same procedure as that of Example 1 and thereby to obtain the title compound as white amorphous solid.

¹H-NMR (400 MHz, CDCl₃) δ: 1.04-2.36 (26H, m), 1.24 (3H, s), 1.37 (9H, s), 1.53 (3H, s), 1.55 (3H, s), 1.70 (3H, s), 2.38 (3H, s), 2.78 (1H, dd, J=12.5, 5.6 Hz), 2.85 (1H, dd, J=12.4, 4.5 Hz), 3.08 (1H, d, J=5.9 Hz), 3.36-3.43 (4H, m), 3.96 (1H, d, J=7.8 Hz), 4.27-4.44 (3H, m), 4.83-4.87 (3H, m), 5.04 (1H, d, J=10.3 Hz), 5.10 (1H, d, J=7.6 Hz), 5.68 (1H, dd, J=10.3, 4.2 Hz), 5.79 (1H, d, J=6.1 Hz), 5.94 (1H, d, J=5.9 Hz), 6.03 (1H, t, J=9.1 Hz), 6.08 (1H, d, J=10.3 Hz), 7.52 (2H, t, J=7.6 Hz), 7.62 (1H, t, J=7.6 Hz), 8.17 (2H, d, J=7.6 Hz).

### Step 3: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-6,11-dien-13-yl

### (2R,3R)-3-(tert-butoxycarbonylamino)-4,4-difluoro-2-hydroxybutyrate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.22 (3H, s), 1.23-2.39 (6H, m), 1.40 (9H, s), 1.52 (3H, s), 1.57 (3H, s), 1.69 (3H, s), 2.38 (3H, s), 2.78 (1H, dd, J=12.5, 5.6 Hz), 2.86 (1H, dd, J=12.4, 4.3 Hz), 3.08 (1H, d, J=5.6 Hz), 3.38 (4H, t, J=7.1 Hz), 3.93 (1H, d, J=7.4 Hz), 4.26 (1H, d, J=8.1 Hz), 4.35 (1H, d, J=8.3 Hz), 4.46-4.56 (1H, m), 4.64 (1H, s), 4.84-4.90 (2H, m), 5.14 (1H, d, J=7.6 Hz), 5.22 (1H, d, J=9.8 Hz), 5.70 (1H, dd, J=10.3, 4.0 Hz), 5.89 (1H, d, J=4.9 Hz), 5.97 (1H, d, J=5.6 Hz), 6.06-6.13 (1H, m), 6.09 (1H, d, J=10.3 Hz), 7.50 (2H, t, J=7.6 Hz), 7.62 (1H, t, J=7.6 Hz), 8.15 (2H, d, J=7.6 Hz).

FAB-MS; m/z: 847 (M+H)⁺.

### Example 51

### (1S,2S,3R,4S,5R,8R,9S,10R,13S)-4-(Acetoxy)-2-(benzoyloxy)-5,20-epoxy-1-hydroxy-9,10-{(1S)-2-[(2R)-2-hydroxymethylazetidino]ethylidenedioxy}tax-11-en-13-yl

### (2R,3S)-3-(tert-butoxycarbonylamino)-3-(3-fluoro-2-pyridyl)-2-hydroxypropionate

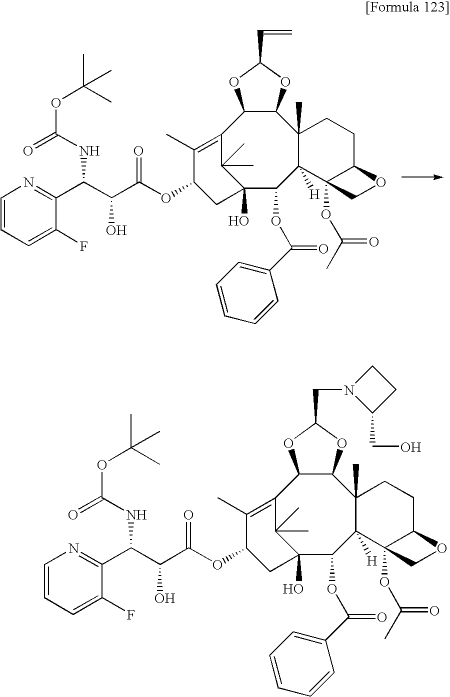

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-3-(3-fluoro-2-pyridyl)-2-hydroxypropionate and the compound obtained in Reference Example 26 were used as starting materials to perform the same procedure as that of Example 1 and thereby to obtain the title compound as white amorphous solid.

¹H-NMR (400 MHz, CDCl₃) δ: 1.25-2.48 (11H, m), 1.29 (3H, s), 1.40 (9H, s), 1.49 (3H, s), 1.61 (3H, s), 1.78 (3H, s), 2.34 (3H, s), 2.79 (1H, dd, J=12.7, 5.6 Hz), 2.92-2.96 (2H, m), 3.08 (1H, dd, J=16.4, 8.5 Hz), 3.43-3.52 (3H, m), 3.65 (1H, dd, J=11.6, 3.1 Hz), 4.15 (1H, d, J=7.1 Hz), 4.22 (1H, d, J=8.5 Hz), 4.32 (1H, d, J=8.5 Hz), 4.68 (1H, d, J=2.7 Hz), 4.88 (1H, dd, J=5.6, 4.4 Hz), 4.91 (1H, s), 5.22 (1H, d, J=7.1 Hz), 5.66 (1H, d, J=7.8 Hz), 5.99 (1H, d, J=5.1 Hz), 6.09 (1H, t, J=8.3 Hz), 6.22 (1H, d, J=8.5 Hz), 7.28-7.33 (1H, m), 7.43-7.49 (3H, m), 7.58-7.62 (1H, m), 8.12-8.15 (2H, m), 8.40 (1H, d, J=4.6 Hz).

FAB-MS; m/z: 924 (M+H)⁺.

### Example 52

### (1S,2S,3R,4S,5R,8R,9S,10R,13S)-4-(Acetoxy)-2-(benzoyloxy)-5,20-epoxy-1-hydroxy-9,10-{(1S)-2-[(2R)-2-methoxymethylazetidino]ethylidenedioxy}tax-11-en-13-yl

### (2R,3S)-3-(tert-butoxycarbonylamino)-3-(3-fluoro-2-pyridyl)-2-hydroxypropionate

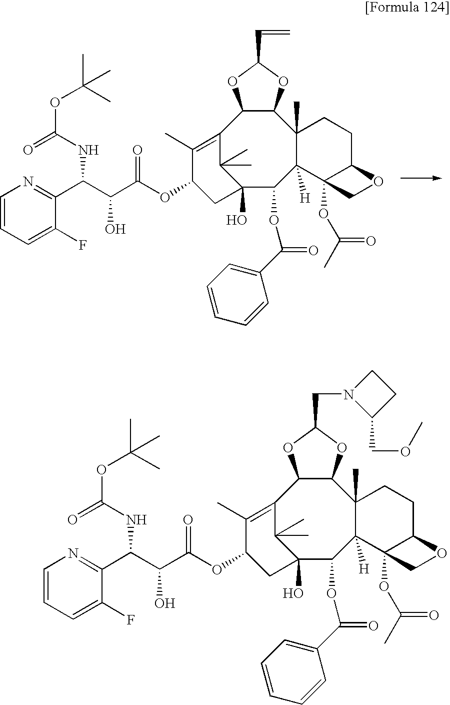

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-3-(3-fluoro-2-pyridyl)-2-hydroxypropionate and the compound obtained in Reference Example 27, Step 2 were used as starting materials to perform the same procedure as that of Example 1 and thereby to obtain the title compound as white amorphous solid.

¹H-NMR (400 MHz, CDCl₃) δ: 1.26-2.48 (9H, m), 1.28 (3H, s), 1.40 (9H, s), 1.49 (3H, s), 1.62 (3H, s), 1.78 (3H, s), 2.34 (3H, s), 2.70 (1H, dd, J=12.7, 6.6 Hz), 2.93 (1H, d, J=5.1 Hz), 2.97-3.08 (2H, m), 3.37 (3H, s), 3.43-3.56 (4H, m), 4.12 (1H, d, J=7.3 Hz), 4.16 (1H, brs), 4.23 (1H, d, J=8.3 Hz), 4.33 (1H, d, J=8.3 Hz), 4.68 (1H, s), 4.89-4.91 (2H, m), 5.21 (1H, d, J=7.1 Hz), 5.65 (1H, d, J=8.5 Hz), 6.00 (1H, d, J=5.1 Hz), 6.08 (1H, t, J=8.8 Hz), 6.21 (1H, d, J=9.3 Hz), 7.28-7.33 (1H, m), 7.42-7.49 (3H, m), 7.60 (1H, t, J=7.4 Hz), 8.12-8.15 (2H, m), 8.40 (1H, d, J=4.9 Hz).

FAB-MS; m/z: 938 (M+H)⁺.

### Example 53

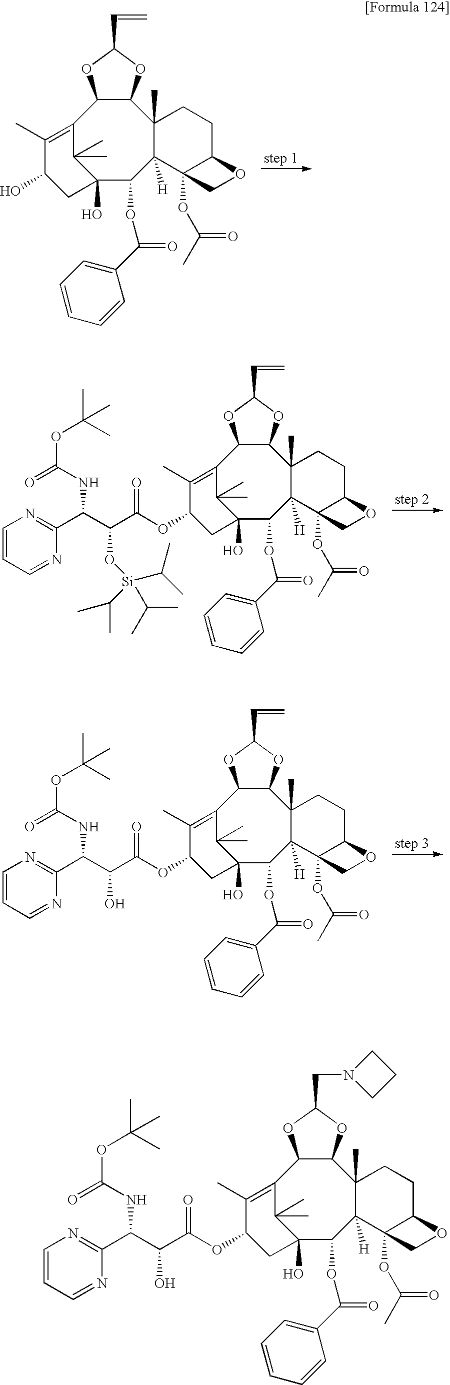

### Step 1: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3S)-3-(tert-butoxycarbonylamino)-3-(2-pyrimidinyl)-2-[(triisopropylsilyl)oxy]propionate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-11-ene and the compound obtained in Reference Example 28, Step 6 were used as starting materials to perform the same procedure as that of Example 31, Step 1 and thereby to obtain the title compound as white amorphous solid.

¹H-NMR (400 MHz, CDCl₃) δ: 0.85-0.92 (21H, m), 1.24-2.08 (5H, m), 1.34 (3H, s), 1.40 (9H, s), 1.53 (3H, s), 1.66 (3H, s), 1.84 (3H, s), 2.32-2.45 (2H, m), 2.58 (3H, s), 2.99 (1H, d, J=5.1 Hz), 4.21-4.24 (2H, m), 4.35 (1H, d, J=8.3 Hz), 4.98 (1H, s), 5.22 (1H, d, J=6.3 Hz), 5.29 (1H, d, J=7.1 Hz), 5.34 (1H, d, J=1.7 Hz), 5.44-5.49 (2H, m), 5.58 (1H, d, J=17.3 Hz), 5.85 (1H, d, J=10.0 Hz), 5.97-6.05 (2H, m), 6.12 (1H, t, J=8.8 Hz), 7.22 (1H, t, J=4.9 Hz), 7.45 (2H, t, J=7.6 Hz), 7.55-7.59 (1H, m), 8.14-8.16 (2H, m), 8.73 (2H, d, J=4.9 Hz).

FAB-MS; m/z: 990 (M+H)⁺, 1012 (M+Na)⁺.

### Step 2: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3S)-3-(tert-butoxycarbonylamino)-2-hydroxy-3-(2-pyrimidinyl)propionate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.24-2.25 (6H, m), 1.30 (3H, s), 1.42 (9H, s), 1.51 (3H, s), 1.65 (3H, s), 1.84 (3H, s), 2.43-2.49 (1H, m), 2.46 (3H, s), 2.97 (1H, d, J=5.1 Hz), 3.91 (1H, s), 4.21-4.24 (2H, m), 4.34 (1H, d, J=8.3 Hz), 4.95 (1H, s), 5.00 (1H, d, J=4.2 Hz), 5.24 (1H, d, J=6.1 Hz), 5.32 (1H, d, J=7.1 Hz), 5.44-5.48 (2H, m), 5.58 (1H, d, J=17.3 Hz), 5.97-6.14 (4H, m), 7.24-7.26 (1H, m), 7.47 (2H, t, J=7.7 Hz), 7.57-7.61 (1H, m), 8.13-8.15 (2H, m), 8.76 (2H, d, J=4.9 Hz).

FAB-MS; m/z: 834 (M+H)⁺, 856 (M+Na)⁺.

### Step 3: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3S)-3-(tert-butoxycarbonylamino)-2-hydroxy-3-(2-pyrimidinyl)propionate

The compound obtained in Step 2 mentioned above was used as a starting material to perform the same procedure as that of Example 1 and thereby to obtain the title compound as white amorphous solid.

¹H-NMR (400 MHz, CDCl₃) δ: 1.26-2.24 (9H, m), 1.28 (3H, s), 1.42 (9H, s), 1.50 (3H, s), 1.62 (3H, s), 1.81 (3H, s), 2.42-2.48 (1H, m), 2.45 (3H, s), 2.74 (1H, dd, J=12.5, 5.8 Hz), 2.82 (1H, dd, J=12.5, 4.4 Hz), 2.95 (1H, d, J=5.1 Hz), 3.32-3.40 (4H, m), 4.14 (1H, d, J=7.1 Hz), 4.22 (1H, d, J=8.5 Hz), 4.34 (1H, d, J=8.5 Hz), 4.87 (1H, dd, J=5.8, 4.4 Hz), 4.94 (1H, s), 4.99 (1H, d, J=2.0 Hz), 5.22 (1H, d, J=6.8 Hz), 5.44 (1H, dd, J=8.8, 1.5 Hz), 6.00 (1H, d, J=5.1 Hz), 6.06-6.13 (2H, m), 7.25 (1H, t, J=4.9 Hz), 7.46 (2H, t, J=7.7 Hz), 7.57-7.61 (1H, m), 8.13-8.15 (2H, m), 8.75 (2H, d, J=4.9 Hz).

FAB-MS; m/z: 877 (M+H)⁺.

### Example 54

### (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3S)-3-(tert-butoxycarbonylamino)-4,4-dimethyl-2-hydroxypentanoate

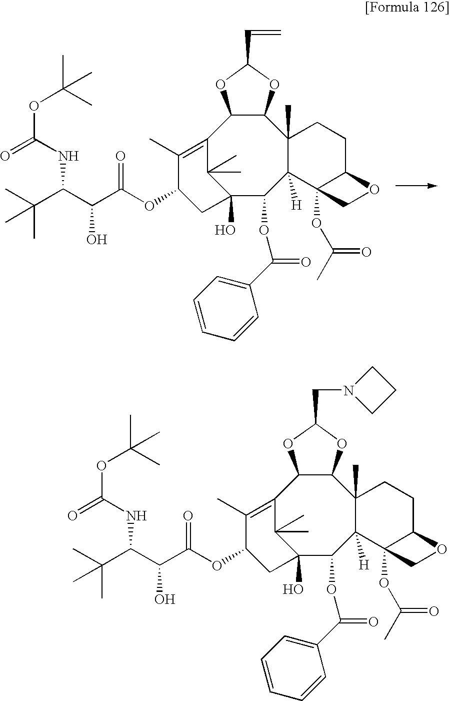

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-4,4-dimethyl-2-hydroxypentanoate (Bioorg. Med. Chem. Lett., 2004, 14, 3209) was used as a starting material to perform the same procedure as that of Example 1 and thereby to obtain the title compound as white amorphous solid.

¹H-NMR (400 MHz, CDCl₃) δ: 1.04 (9H, s), 1.24 (3H, s), 1.34-2.40 (10H, m), 1.38 (9H, s), 1.49 (3H, s), 1.61 (3H, s), 1.78 (3H, s), 2.34 (3H, s), 2.74 (1H, dd, J=12.5, 5.9 Hz), 2.83 (1H, dd, J=12.5, 4.4 Hz), 2.92 (1H, d, J=5.1 Hz), 3.31-3.39 (4H, m), 3.93 (1H, d, J=10.5 Hz), 4.12 (1H, d, J=7.3 Hz), 4.25 (1H, d, J=8.5 Hz), 4.33 (1H, d, J=8.5 Hz), 4.60 (1H, s), 4.87 (1H, dd, J=5.9, 4.4 Hz), 4.93 (1H, s), 5.10 (1H, d, J=10.5 Hz), 5.22 (1H, d, J=6.8 Hz), 5.99-6.06 (2H, m), 7.48 (2H, t, J=7.7 Hz), 7.59-7.63 (1H, m), 8.12-8.14 (2H, m).

FAB-MS; m/z: 855 (M+H)⁺.

### Example 55

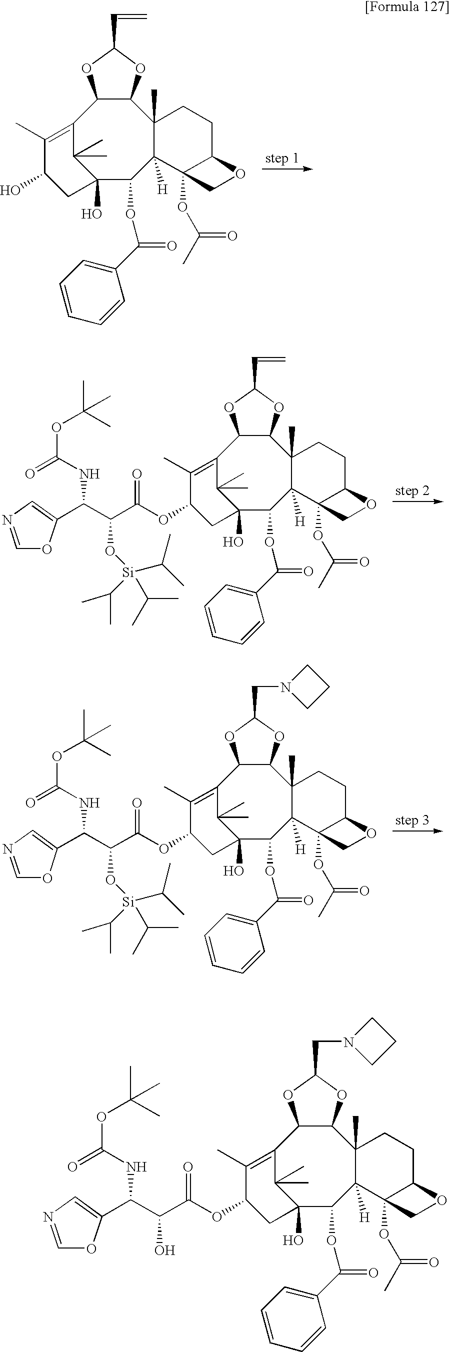

### Step 1: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3R)-3-(tert-butoxycarbonylamino)-3-(5-oxazolyl)-2-[(triisopropylsilyl)oxy]propionate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-11-ene and the compound obtained in Reference Example 29, Step 6 were used as starting materials to perform the same procedure as that of Example 31, Step 1 and thereby to obtain the title compound as white amorphous solid.

¹H-NMR (400 MHz, CDCl₃) δ: 0.95-1.04 (21H, m), 1.30 (3H, s), 1.41 (9H, s), 1.52 (3H, s), 1.65 (3H, s), 1.80 (3H, s), 1.86-2.36 (7H, m), 2.41 (3H, s), 2.96 (1H, d, J=4.9 Hz), 4.21 (2H, d, J=7.8 Hz), 4.35 (1H, d, J=8.5 Hz), 4.93-4.97 (2H, m), 5.21 (1H, d, J=6.6 Hz), 5.26-5.33 (2H, m), 5.40 (1H, d, J=10.7 Hz), 5.46 (1H, d, J=10.7 Hz), 5.58 (1H, d, J=17.3 Hz), 5.96-6.13 (3H, m), 7.02 (1H, s), 7.47 (2H, t, J=7.3 Hz), 7.59 (1H, t, J=7.3 Hz), 7.87 (1H, s), 8.14 (2H, d, J=8.5 Hz).

### Step 2: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3R)-3-(tert-butoxycarbonylamino)-3-(5-oxazolyl)-2-[(triisopropylsilyl)oxy]propionate

The compound obtained in Step 1 mentioned above was used as a starting material to perform the same procedure as that of Example 1 and thereby to obtain the title compound as white amorphous solid.

¹H-NMR (400 MHz, CDCl₃) δ: 0.95-1.01 (21H, m), 1.28 (3H, s), 1.40 (9H, s), 1.51 (3H, s), 1.54-2.18 (9H, m), 1.61 (3H, s), 1.77 (3H, s), 2.40 (3H, s), 2.73 (1H, dd, J=12.7, 5.9 Hz), 2.81 (1H, dd, J=12.0, 4.4 Hz), 2.94 (1H, d, J=4.9 Hz), 3.32-3.39 (4H, m), 4.14 (1H, d, J=7.1 Hz), 4.21 (1H, d, J=8.3 Hz), 4.34 (1H, d, J=8.8 Hz), 4.84 (1H, t, J=5.6 Hz), 4.94 (2H, brs), 5.18 (1H, d, J=7.6 Hz), 5.29 (1H, d, J=10.5 Hz), 5.39 (1H, d, J=10.0 Hz), 5.97 (1H, d, J=5.1 Hz), 6.07 (1H, t, J=8.3 Hz), 7.01 (1H, s), 7.47 (2H, t, J=7.7 Hz), 7.59 (1H, t, J=7.6 Hz), 7.86 (1H, s), 8.14 (2H, d, J=7.8 Hz).

FAB-MS; m/z: 1022 (M+H)⁺.

### Step 3: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-1′-en-13-yl

### (2R,3R)-3-(tert-butoxycarbonylamino)-2-hydroxy-3-(5-oxazolyl)propionate

The compound obtained in Step 2 mentioned above was used as a starting material to perform the same procedure as that of Example 31, Step 2 and thereby to obtain the title compound as white amorphous solid.

¹H-NMR (400 MHz, CDCl₃) δ: 1.26 (3H, s), 1.43 (9H, s), 1.49 (3H, s), 1.58-2.17 (10H, m), 1.61 (3H, s), 1.73 (3H, s), 2.32 (3H, s), 2.74 (1H, dd, J=12.5, 5.6 Hz), 2.83 (1H, dd, J=12.5, 4.6 Hz), 2.92 (1H, d, J=4.9 Hz), 3.31-3.40 (4H, m), 4.10 (1H, d, J=7.1 Hz), 4.25 (1H, d, J=8.3 Hz), 4.33 (1H, d, J=8.5 Hz), 4.69 (1H, s), 4.87 (1H, dd, J=5.6, 4.4 Hz), 4.92 (1H, brs), 5.22 (1H, d, J=7.6 Hz), 5.46-5.55 (2H, m), 6.01 (1H, d, J=5.1 Hz), 6.13 (1H, t, J=8.3 Hz), 7.07 (1H, s), 7.48 (2H, t, J=7.7 Hz), 7.61 (1H, t, J=7.6 Hz), 7.85 (1H, s), 8.13 (2H, d, J=8.1 Hz).

FAB-MS; m/z: 866 (M+H)⁺.

### Example 56

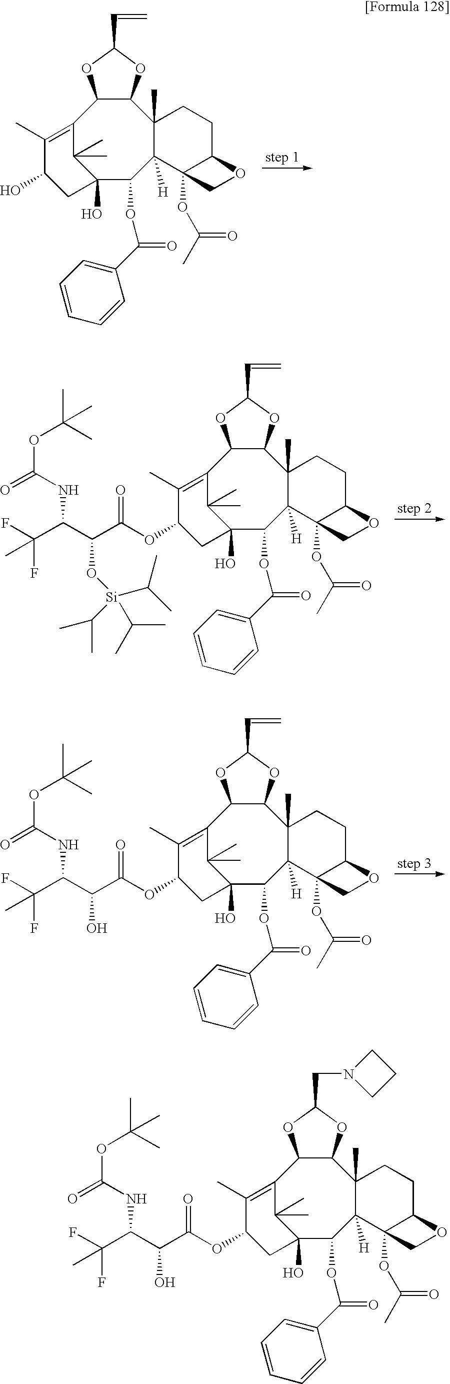

### Step 1: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3R)-3-(tert-butoxycarbonylamino)-4,4-difluoro-2-[(triisopropylsilyl)oxy]pentanoate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-11-ene and the compound obtained in Reference Example 30, Step 6 were used as starting materials to perform the same procedure as that of Example 31, Step 1 and thereby to obtain the title compound as white amorphous solid.

¹H-NMR (400 MHz, CDCl₃) δ: 1.05-1.17 (21H, m), 1.24-2.31 (8H, m), 1.29 (3H, s), 1.39 (9H, s), 1.51 (3H, s), 1.64 (3H, s), 1.68 (3H, t, J=19.0 Hz), 1.79 (3H, s), 1.85 (3H, s), 2.93 (1H, d, J=5.4 Hz), 4.20 (2H, t, J=5.0 Hz), 4.35 (1H, d, J=7.8 Hz), 4.93 (1H, s), 5.03 (1H, s), 5.14-5.22 (2H, m), 5.26 (1H, d, J=7.3 Hz), 5.45 (1H, d, J=10.5 Hz), 5.57 (1H, d, J=17.1 Hz), 5.95-6.04 (3H, m), 7.49 (2H, t, J=7.8 Hz), 7.61 (1H, t, J=7.2 Hz), 8.15 (2H, d, J=7.3 Hz).

### Step 2: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3R)-3-(tert-butoxycarbonylamino)-4,4-difluoro-2-hydroxypentanoate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.22-2.41 (10H, m), 1.25 (3H, s), 1.42 (9H, s), 1.64 (3H, s), 1.72 (3H, t, J=19.0 Hz), 1.78 (3H, s), 2.35 (3H, s), 2.94 (1H, d, J=4.6 Hz), 4.18 (1H, d, J=7.3 Hz), 4.25 (1H, d, J=8.3 Hz), 4.34 (1H, d, J=8.5 Hz), 4.46-4.49 (1H, m), 4.50-4.61 (1H, m), 4.76-4.78 (1H, m), 4.93 (1H, s), 5.25 (1H, d, J=6.6 Hz), 5.31 (2H, d, J=7.8 Hz), 5.47 (1H, d, J=10.7 Hz), 5.58 (1H, d, J=17.3 Hz), 5.96-6.06 (2H, m), 6.10-6.16 (1H, m), 7.49 (2H, t, J=7.7 Hz), 7.59-7.64 (1H, m), 8.12-8.15 (2H, m).

### Step 3: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-1′-en-13-yl

### (2R,3R)-3-(tert-butoxycarbonylamino)-4,4-difluoro-2-hydroxypentanoate

The compound obtained in Step 2 mentioned above was used as a starting material to perform the same procedure as that of Example 1 and thereby to obtain the title compound as white amorphous solid.

¹H-NMR (400 MHz, CDCl₃) δ: 1.22-2.38 (10H, m), 1.24 (3H, s), 1.41 (9H, s), 1.49 (3H, s), 1.61 (3H, s), 1.72 (3H, t, J=19.3 Hz), 1.76 (3H, s), 2.34 (3H, s), 2.75 (1H, dd, J=12.2, 5.6 Hz), 2.84 (1H, dd, J=12.5, 4.2 Hz), 2.92 (1H, d, J=5.1 Hz), 3.34-3.41 (4H, m), 4.11 (1H, d, J=7.1 Hz), 4.24 (1H, d, J=8.3 Hz), 4.34 (1H, d, J=8.3 Hz), 4.49-4.60 (1H, m), 4.76 (1H, s), 4.89 (1H, t, J=4.9 Hz), 4.93 (1H, s), 5.22 (1H, d, J=6.8 Hz), 5.31 (1H, d, J=10.5 Hz), 6.00 (1H, d, J=5.1 Hz), 6.08-6.14 (1H, m), 7.49 (2H, t, J=7.3 Hz), 7.59-7.64 (1H, m), 8.11-8.15 (2H, m).

FAB-MS; m/z: 863 (M+H)⁺.

### Example 57

### (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-11-en-13-yl

### (2R,3R)-3-(tert-butoxycarbonylamino)-4,4-difluoro-2-hydroxypentanoate

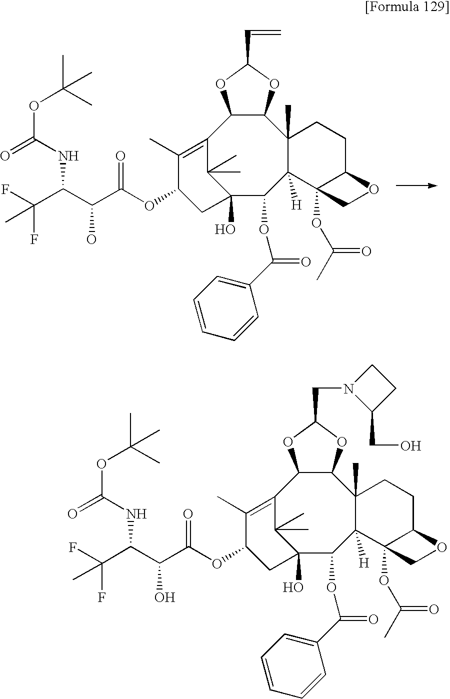

The compound obtained in Example 56, Step 2 and the compound obtained in Reference Example 25 were used as starting materials to perform the same procedure as that of Example 1 and thereby to obtain the title compound as white amorphous solid.

¹H-NMR (400 MHz, CDCl₃) δ: 1.21-2.38 (11H, m), 1.23 (3H, s), 1.42 (9H, s), 1.49 (3H, s), 1.59 (3H, s), 1.72 (3H, t, J=19.0 Hz), 1.76 (3H, s), 2.34 (3H, s), 2.84 (1H, dd, J=12.8, 4.5 Hz), 2.88-2.94 (2H, m), 3.12 (1H, q, J=8.2 Hz), 3.43 (1H, dd, J=11.7, 2.4 Hz), 3.47-3.55 (2H, m), 3.67 (1H, dd, J=11.5, 2.9 Hz), 4.13 (1H, d, J=7.1 Hz), 4.24 (1H, d, J=8.3 Hz), 4.33 (1H, d, J=8.3 Hz), 4.49-4.60 (1H, m), 4.77 (1H, s), 4.87-4.94 (2H, m), 5.23 (1H, d, J=7.1 Hz), 5.31 (1H, d, J=10.3 Hz), 5.99 (1H, d, J=4.9 Hz), 6.08-6.14 (1H, m), 7.49 (2H, t, J=7.7 Hz), 7.59-7.64 (1H, m), 8.11-8.15 (2H, m).

FAB-MS; m/z: 893 (M+H)⁺.

### Example 58

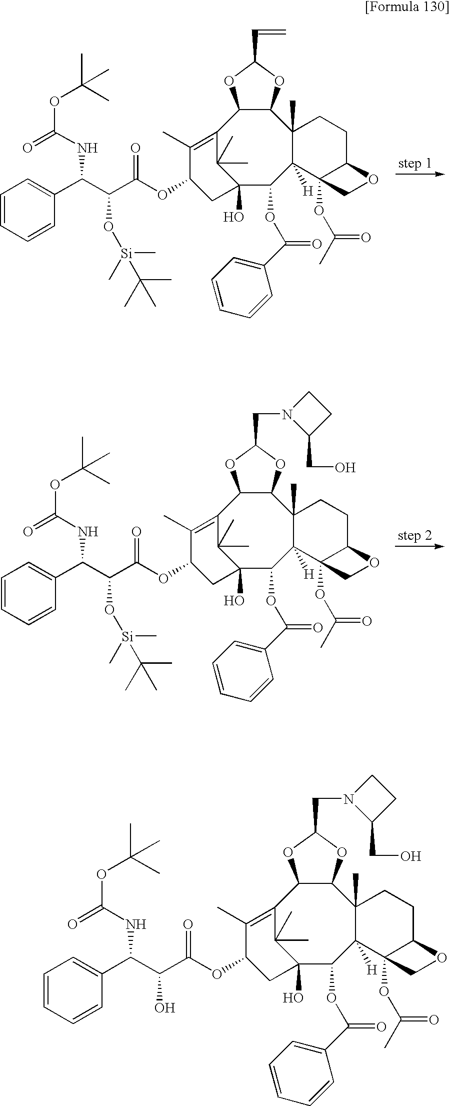

### Step 1: (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-11-en-13-yl

### (2R,3S)-3-(tert-butoxycarbonylamino)-2-[(tert-butyldimethylsilyl)oxy]-3-phenylpropionate

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-2-[(tert-butyldimethylsilyl)oxy]-3-phenylpropionate and the compound obtained in Reference Example 25 were used as starting materials to perform the same procedure as that of Example 1 and thereby to obtain the title compound as white amorphous solid.

¹H-NMR (400 MHz, CDCl₃) δ: −0.34 (3H, s), −0.11 (3H, s), 0.74 (9H, s), 1.32 (3H, s), 1.39 (9H, s), 1.52 (3H, s), 1.60 (3H, s), 1.66-2.33 (10H, m), 1.76 (3H, s), 2.51 (3H, s), 2.81 (1H, dd, J=12.7, 4.6 Hz), 2.90 (1H, dd, J=12.7, 5.1 Hz), 2.94 (1H, d, J=4.9 Hz), 3.06-3.15 (1H, m), 3.39-3.53 (3H, m), 3.64 (1H, dd, J=12.0, 2.9 Hz), 4.16 (1H, d, J=7.3 Hz), 4.24 (1H, d, J=8.5 Hz), 4.34 (1H, d, J=7.8 Hz), 4.56 (1H, s), 4.86 (1H, t, J=4.9 Hz), 4.96 (1H, s), 5.20 (1H, d, J=6.8 Hz), 5.32 (1H, d, J=9.0 Hz), 5.48 (1H, d, J=9.0 Hz), 5.96 (1H, d, J=4.9 Hz), 6.20 (1H, t, J=9.3 Hz), 7.25-7.39 (5H, m), 7.48 (2H, t, J=7.1 Hz), 7.60 (1H, t, J=7.6 Hz), 8.15 (2H, d, J=7.1 Hz).

FAB-MS; m/z: 1019 (M+H)⁺.

### Step 2: (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-11-en-13-yl

### (2R,3S)-3-(tert-butoxycarbonylamino)-2-hydroxy-3-phenylpropionate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.26 (3H, s), 1.41 (9H, s), 1.47 (3H, s), 1.58 (3H, s), 1.64 (3H, s), 1.76-2.43 (11H, m), 2.27 (3H, s), 2.83 (1H, dd, J=12.7, 4.6 Hz), 2.86-2.92 (2H, m), 3.06-3.14 (1H, m), 3.39-3.53 (3H, m), 3.66 (1H, dd, J=12.0, 2.9 Hz), 4.10 (1H, d, J=7.3 Hz), 4.24 (1H, d, J=8.5 Hz), 4.31 (1H, d, J=7.8 Hz), 4.63 (1H, s), 4.88 (1H, t, J=4.9 Hz), 4.91 (1H, s), 5.21 (1H, d, J=6.8 Hz), 5.31 (1H, d, J=9.0 Hz), 5.70 (1H, d, J=9.0 Hz), 5.98 (1H, d, J=4.9 Hz), 6.06 (1H, t, J=9.3 Hz), 7.25-7.51 (7H, m), 7.60 (1H, t, J=7.6 Hz), 8.12 (2H, d, J=7.1 Hz).

FAB-MS; m/z: 905 (M+H)⁺.

### Example 59

### (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-11-en-13-yl

### (2R,3R)-3-(tert-butoxycarbonylamino)-4-fluoro-2-hydroxybutyrate

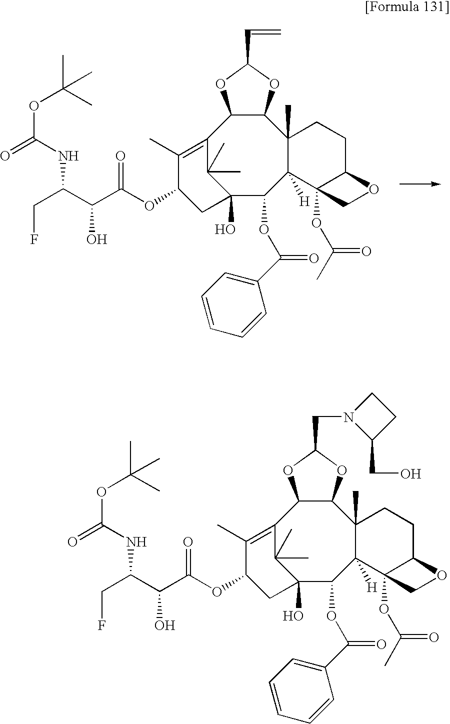

The compound obtained in Example 34, Step 2 and the compound obtained in Reference Example 25 were used as starting materials to perform the same procedure as that of Example 1 and thereby to obtain the title compound as white amorphous solid.

¹H-NMR (400 MHz, CDCl₃) δ: 1.22-2.05 (9H, m), 1.23 (3H, s), 1.41 (9H, s), 1.49 (3H, s), 1.59 (3H, s), 1.79 (3H, s), 2.22-2.40 (2H, m), 2.33 (3H, s), 2.83 (1H, dd, J=12.7, 4.6 Hz), 2.88-2.93 (2H, m), 3.07-3.13 (1H, m), 3.41-3.52 (3H, m), 3.65 (1H, dd, J=11.4, 3.1 Hz), 4.13 (1H, d, J=7.3 Hz), 4.24 (1H, d, J=8.5 Hz), 4.33 (1H, d, J=8.5 Hz), 4.41-4.64 (4H, m), 4.89 (1H, t, J=4.9 Hz), 4.93 (1H, s), 5.13 (1H, d, J=9.0 Hz), 5.24 (1H, d, J=7.1 Hz), 5.99 (1H, d, J=5.1 Hz), 6.06 (1H, t, J=8.3 Hz), 7.48 (2H, t, J=7.7 Hz), 7.59-7.63 (1H, m), 8.12-8.14 (2H, m).

FAB-MS; m/z: 861 (M+H)⁺.

### Example 60

### (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-11-en-13-yl

### (2R,3R)-3-(tert-butoxycarbonylamino)-2-hydroxy-3-(2-thiazolyl)propionate

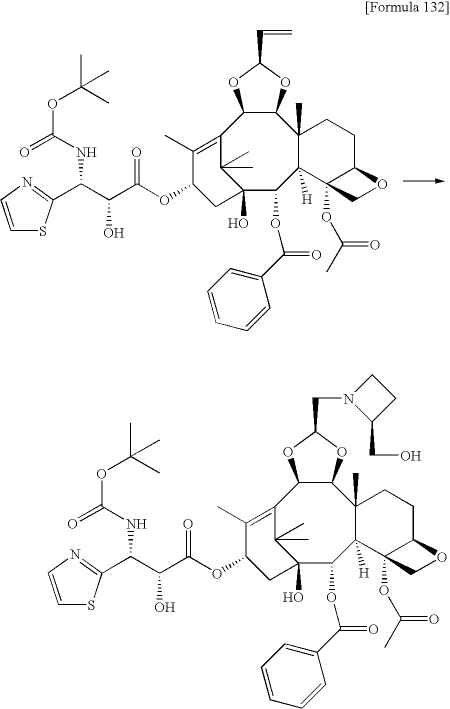

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3R)-3-(tert-butoxycarbonylamino)-2-hydroxy-3-(2-thiazolyl)propionate and the compound obtained in Reference Example 25 were used as starting materials to perform the same procedure as that of Example 1 and thereby to obtain the title compound as white amorphous solid.

¹H-NMR (400 MHz, CDCl₃) δ: 1.23-2.37 (11H, m), 1.24 (3H, s), 1.45 (9H, s), 1.49 (3H, s), 1.58 (3H, s), 1.73 (3H, s), 2.34 (3H, s), 2.82 (1H, dd, J=12.9, 4.6 Hz), 2.87-2.93 (2H, m), 3.07-3.13 (1H, m), 3.42 (1H, dd, J=11.5, 2.4 Hz), 3.46-3.52 (2H, m), 3.65 (1H, dd, J=11.5, 3.1 Hz), 4.12 (1H, d, J=7.1 Hz), 4.22 (1H, d, J=8.3 Hz), 4.32 (1H, d, J=8.3 Hz), 4.87 (1H, t, J=5.0 Hz), 4.92 (1H, s), 5.00 (1H, d, J=2.4 Hz), 5.22 (1H, d, J=6.8 Hz), 5.60 (1H, d, J=9.5 Hz), 5.91-5.98 (2H, m), 6.12 (1H, t, J=8.3 Hz), 7.31 (1H, d, J=3.2 Hz), 7.47 (2H, t, J=7.7 Hz), 7.58-7.62 (1H, m), 7.76 (1H, d, J=3.2 Hz), 8.11-8.14 (2H, m).

FAB-MS; m/z: 912 (M+H)⁺.

### Example 61

### (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-11-en-13-yl

### (2R,3S)-3-(tert-butoxycarbonylamino)-4,4-dimethyl-2-hydroxypentanoate

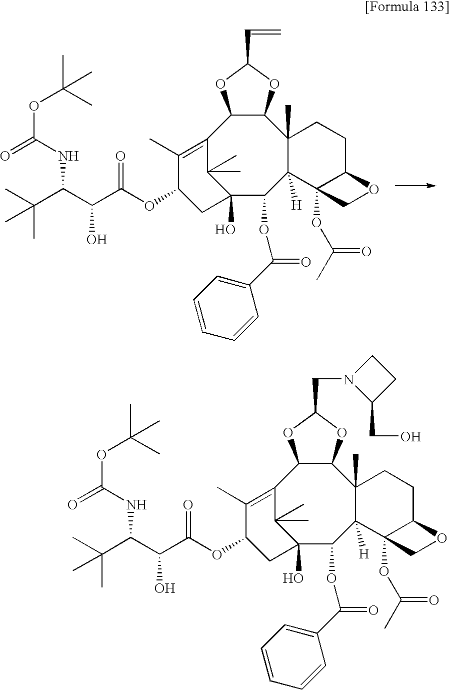

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-4,4-dimethyl-2-hydroxypentanoate and the compound obtained in Reference Example 25 were used as starting materials to perform the same procedure as that of Example 1 and thereby to obtain the title compound as white amorphous solid.

¹H-NMR (400 MHz, CDCl₃) δ: 1.02-2.04 (9H, m), 1.05 (9H, s), 1.23 (3H, s), 1.39 (9H, s), 1.49 (3H, s), 1.58 (3H, s), 1.78 (3H, s), 2.22-2.40 (2H, m), 2.34 (3H, s), 2.83 (1H, dd, J=12.7, 4.6 Hz), 2.87-2.92 (2H, m), 3.07-3.13 (1H, m), 3.42 (1H, dd, J=11.5, 2.4 Hz), 3.46-3.52 (2H, m), 3.66 (1H, dd, J=11.5, 2.9 Hz), 3.93 (1H, d, J=10.5 Hz), 4.13 (1H, d, J=7.1 Hz), 4.24 (1H, d, J=8.5 Hz), 4.32 (1H, d, J=8.5 Hz), 4.60 (1H, s), 4.89 (1H, t, J=4.9 Hz), 4.93 (1H, s), 5.10 (1H, d, J=10.5 Hz), 5.23 (1H, d, J=6.8 Hz), 5.98 (1H, d, J=5.1 Hz), 6.04 (1H, t, J=8.3 Hz), 7.48 (2H, t, J=7.7 Hz), 7.59-7.63 (1H, m), 8.11-8.14 (2H, m).

FAB-MS; m/z: 885 (M+H)⁺.

### Example 62

### (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-11-en-13-yl

### (2R,3S)-3-(tert-butoxycarbonylamino)-2-hydroxy-3-(2-pyrimidinyl)propionate

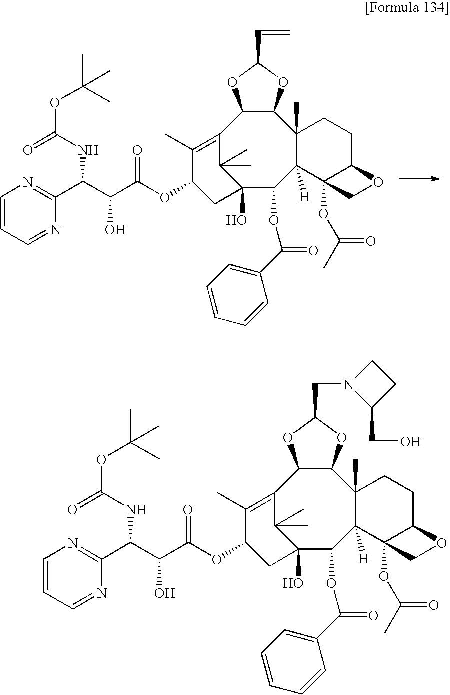

The compound obtained in Example 53, Step 2 and the compound obtained in Reference Example 25 were used as starting materials to perform the same procedure as that of Example 1 and thereby to obtain the title compound as white amorphous solid.

¹H-NMR (400 MHz, CDCl₃) δ: 1.27-2.28 (10H, m), 1.28 (3H, s), 1.42 (9H, s), 1.50 (3H, s), 1.60 (3H, s), 1.81 (3H, s), 2.42-2.48 (1H, m), 2.45 (3H, s), 2.83 (1H, dd, J=12.7, 4.6 Hz), 2.90 (1H, dd, J=12.7, 5.1 Hz), 2.96 (1H, d, J=5.1 Hz), 3.11 (1H, dd, J=16.1, 8.5 Hz), 3.43 (1H, dd, J=11.5, 2.7 Hz), 3.46-3.52 (2H, m), 3.66 (1H, dd, J=11.5, 3.1 Hz), 4.16 (1H, d, J=7.3 Hz), 4.22 (1H, d, J=8.5 Hz), 4.34 (1H, d, J=8.5 Hz), 4.88 (1H, t, J=4.9 Hz), 4.94 (1H, s), 4.99 (1H, d, J=2.0 Hz), 5.24 (1H, d, J=7.1 Hz), 5.44 (1H, dd, J=8.8, 1.5 Hz), 5.99 (1H, d, J=5.1 Hz), 6.07-6.13 (2H, m), 7.24-7.26 (1H, m), 7.47 (2H, t, J=7.7 Hz), 7.57-7.61 (1H, m), 8.13-8.15 (2H, m), 8.75 (2H, d, J=4.9 Hz).

FAB-MS; m/z: 907 (M+H)⁺.

### Example 63

### (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-11-en-13-yl

### (2R,3S)-3-(tert-butoxycarbonylamino)-2-hydroxypentanoate

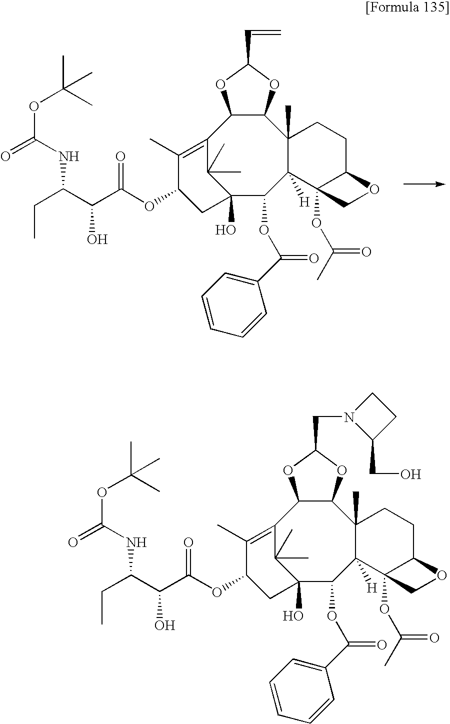

The compound obtained in Example 37, Step 2 and the compound obtained in Reference Example 25 were used as starting materials to perform the same procedure as that of Example 1 and thereby to obtain the title compound as white amorphous solid.

¹H-NMR (400 MHz, CDCl₃) δ: 0.99-2.41 (10H, m), 1.01 (3H, t, J=7.4 Hz), 1.24 (3H, s), 1.40 (9H, s), 1.49 (3H, s), 1.59 (3H, s), 1.78 (3H, s), 2.33 (3H, s), 2.83 (1H, dd, J=12.8, 4.5 Hz), 2.88-2.93 (2H, m), 3.07-3.15 (1H, m), 3.43 (1H, dd, J=11.5, 2.4 Hz), 3.47-3.54 (2H, m), 3.66 (1H, dd, J=11.5, 2.9 Hz), 3.97-4.05 (1H, m), 4.14 (1H, d, J=7.1 Hz), 4.24 (1H, d, J=8.3 Hz), 4.31-4.35 (2H, m), 4.83-4.91 (2H, m), 4.93 (1H, s), 5.24 (1H, d, J=6.8 Hz), 5.99 (1H, d, J=5.1 Hz), 6.02-6.08 (1H, m), 7.48 (2H, t, J=7.7 Hz), 7.59-7.63 (1H, m), 8.11-8.15 (2H, m).

FAB-MS; m/z: 857 (M+H)⁺.

### Example 64

### (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-11-en-13-yl

### (2R,3S)-3-(tert-butoxycarbonylamino)-2-hydroxy-4-methylpentanoate

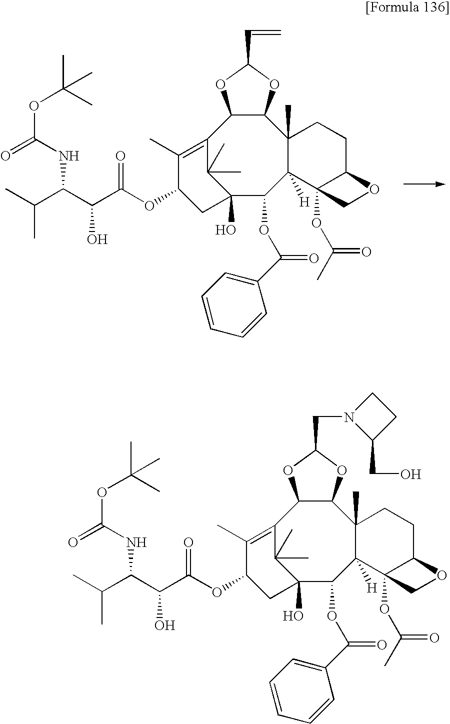

The compound obtained in Example 49, Step 2 and the compound obtained in Reference Example 25 were used as starting materials to perform the same procedure as that of Example 1 and thereby to obtain the title compound as white amorphous solid.

¹H-NMR (400 MHz, CDCl₃) δ: 1.00-2.09 (10H, m), 1.02 (3H, d, J=6.8 Hz), 1.06 (3H, d, J=6.8 Hz), 1.24 (3H, s), 1.38 (9H, s), 1.49 (3H, s), 1.58 (3H, s), 1.79 (3H, s), 2.22-2.40 (2H, m), 2.34 (3H, s), 2.83 (1H, dd, J=12.9, 4.6 Hz), 2.87-2.92 (2H, m), 3.10 (1H, dd, J=16.1, 8.3 Hz), 3.42 (1H, dd, J=11.5, 2.4 Hz), 3.46-3.52 (2H, m), 3.66 (1H, dd, J=11.5, 2.9 Hz), 3.76-3.81 (1H, m), 4.14 (1H, d, J=7.1 Hz), 4.24 (1H, d, J=8.5 Hz), 4.33 (1H, d, J=8.5 Hz), 4.49 (1H, d, J=1.5 Hz), 4.86-4.93 (3H, m), 5.23 (1H, d, J=7.1 Hz), 5.98 (1H, d, J=5.1 Hz), 6.04 (1H, t, J=8.8 Hz), 7.48 (2H, t, J=7.7 Hz), 7.59-7.63 (1H, m), 8.12-8.14 (2H, m).

FAB-MS; m/z: 871 (M+H)⁺.

### Example 65

### (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-11-en-13-yl

### (2R,3R)-4,4-difluoro-2-hydroxy-3-(isopropoxycarbonylamino)butyrate

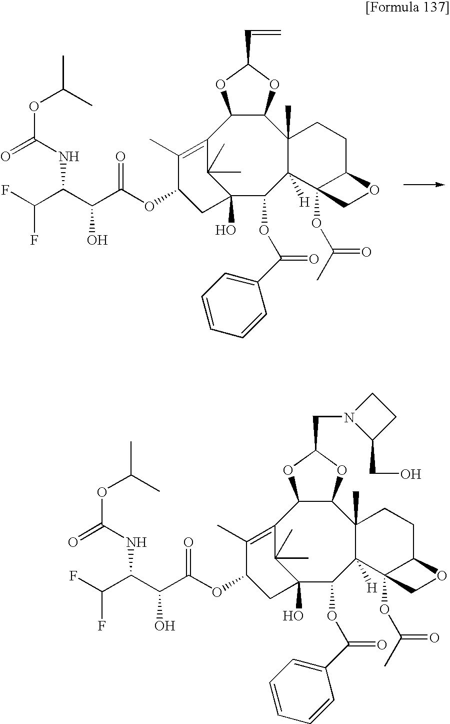

The compound obtained in Example 45, Step 2 and the compound obtained in Reference Example 25 were used as starting materials to perform the same procedure as that of Example 1 and thereby to obtain the title compound as white amorphous solid.

¹H-NMR (400 MHz, CDCl₃) δ: 1.19-2.06 (15H, m), 1.23 (3H, s), 1.48 (3H, s), 1.58 (3H, s), 1.75 (3H, s), 2.23-2.37 (2H, m), 2.33 (3H, s), 2.83 (1H, dd, J=12.9, 4.4 Hz), 2.88-2.93 (2H, m), 3.11 (1H, dd, J=16.1, 8.3 Hz), 3.43 (1H, dd, J=11.4, 2.7 Hz), 3.47-3.53 (2H, m), 3.65 (1H, dd, J=11.4, 3.1 Hz), 4.12 (1H, d, J=7.1 Hz), 4.25 (1H, d, J=8.5 Hz), 4.33 (1H, d, J=8.5 Hz), 4.56-4.65 (2H, m), 4.88-4.93 (3H, m), 5.23 (1H, d, J=6.8 Hz), 5.35 (1H, d, J=10.0 Hz), 5.91 (1H, dt, J=56.2, 4.4 Hz), 6.00 (1H, d, J=5.1 Hz), 6.13 (1H, t, J=8.5 Hz), 7.48 (2H, t, J=7.7 Hz), 7.59-7.64 (1H, m), 8.11-8.14 (2H, m).

FAB-MS; m/z: 865 (M+H)⁺.

### Example 66

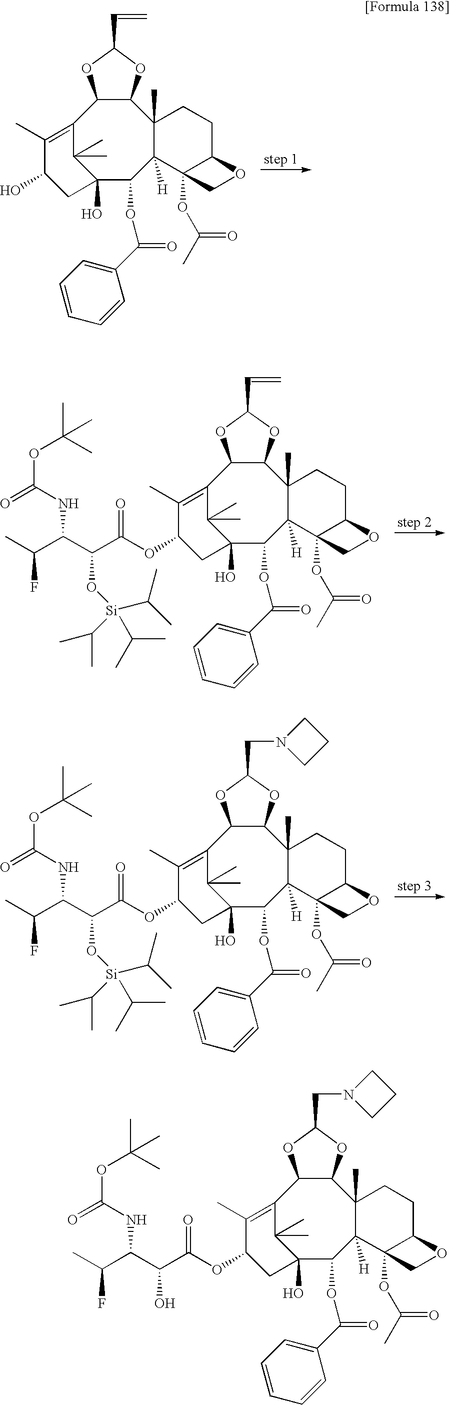

### Step 1: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3R,4S)-3-(tert-butoxycarbonylamino)-4-fluoro-2-[(triisopropylsilyl)oxy]pentanoate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-11-ene and the compound obtained in Reference Example 31, Step 4 were used as starting materials to perform the same procedure as that of Example 31, Step 1 and thereby to obtain the title compound as white amorphous solid.

¹H-NMR (400 MHz, CDCl₃) δ: 1.10-1.16 (21H, m), 1.28 (3H, s), 1.37 (9H, s), 1.44 (3H, dd, J=24.4, 6.1 Hz), 1.51 (3H, s), 1.64 (3H, s), 1.79 (3H, s), 1.83-2.30 (7H, m), 2.34 (3H, s), 2.93 (1H, d, J=5.1 Hz), 4.00-4.09 (1H, m), 4.17-4.22 (2H, m), 4.35 (1H, d, J=8.3 Hz), 4.52-4.74 (1H, m), 4.84 (1H, d, J=10.5 Hz), 4.90 (1H, s), 4.93 (1H, brs), 5.20 (1H, d, J=6.3 Hz), 5.27 (1H, d, J=7.3 Hz), 5.45 (1H, d, J=10.7 Hz), 5.57 (1H, d, J=17.3 Hz), 5.95-6.04 (3H, m), 7.50 (2H, t, J=7.7 Hz), 7.61 (1H, t, J=7.6 Hz), 8.16 (2H, d, J=7.1 Hz).

FAB-MS; m/z: 958 (M+H)⁺.

### Step 2: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3R,4S)-3-(tert-butoxycarbonylamino)-4-fluoro-2-[(triisopropylsilyl)oxy]pentanoate

The compound obtained in Step 1 mentioned above was used as a starting material to perform the same procedure as that of Example 1 and thereby to obtain the title compound as white amorphous solid.

¹H-NMR (400 MHz, CDCl₃) δ: 1.09-1.15 (21H, m), 1.26 (3H, s), 1.37 (9H, s), 1.43 (3H, dd, J=24.6, 6.3 Hz), 1.50 (3H, s), 1.61 (3H, s), 1.76 (3H, s), 1.82-2.30 (9H, m), 2.33 (3H, s), 2.73 (1H, dd, J=12.2, 5.6 Hz), 2.80 (1H, dd, J=12.2, 4.1 Hz), 2.91 (1H, d, J=5.9 Hz), 3.32-3.38 (4H, m), 4.00-4.08 (1H, m), 4.13 (1H, d, J=7.3 Hz), 4.19 (1H, d, J=8.0 Hz), 4.35 (1H, d, J=8.3 Hz), 4.52-4.73 (1H, m), 4.81-4.86 (2H, m), 4.89 (1H, s), 4.92 (1H, brs), 5.17 (1H, d, J=7.6 Hz), 5.94-6.02 (2H, m), 7.50 (2H, t, J=7.8 Hz), 7.61 (1H, t, J=7.3 Hz), 8.16 (2H, d, J=8.0 Hz).

FAB-MS; m/z: 1001 (M+H)⁺.

### Step 3: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3R,4S)-3-(tert-butoxycarbonylamino)-4-fluoro-2-hydroxypentanoate

The compound obtained in Step 2 mentioned above was used as a starting material to perform the same procedure as that of Example 31, Step 2 and thereby to obtain the title compound as white amorphous solid.

¹H-NMR (400 MHz, CDCl₃) δ: 1.26 (3H, s), 1.39 (9H, s), 1.45 (3H, dd, J=24.6, 6.3 Hz), 1.49 (3H, s), 1.61 (3H, s), 1.77 (3H, s), 1.80-2.16 (10H, m), 2.34 (3H, s), 2.74 (1H, dd, J=12.2, 5.6 Hz), 2.83 (1H, dd, J=12.2, 4.4 Hz), 2.92 (1H, d, J=4.9 Hz), 3.32-3.38 (4H, m), 4.12 (1H, d, J=7.3 Hz), 4.17-4.27 (1H, m), 4.24 (1H, d, J=8.0 Hz), 4.34 (1H, d, J=8.3 Hz), 4.58-4.80 (1H, m), 4.69 (1H, s), 4.87 (1H, t, J=5.1 Hz), 4.93 (1H, brs), 4.97 (1H, d, J=10.2 Hz), 5.22 (1H, d, J=7.6 Hz), 6.00 (1H, d, J=5.4 Hz), 6.08 (1H, t, J=8.3 Hz), 7.49 (2H, t, J=7.8 Hz), 7.61 (1H, t, J=7.3 Hz), 8.14 (2H, d, J=8.0 Hz).

FAB-MS; m/z: 845 (M+H)⁺.

### Example 67

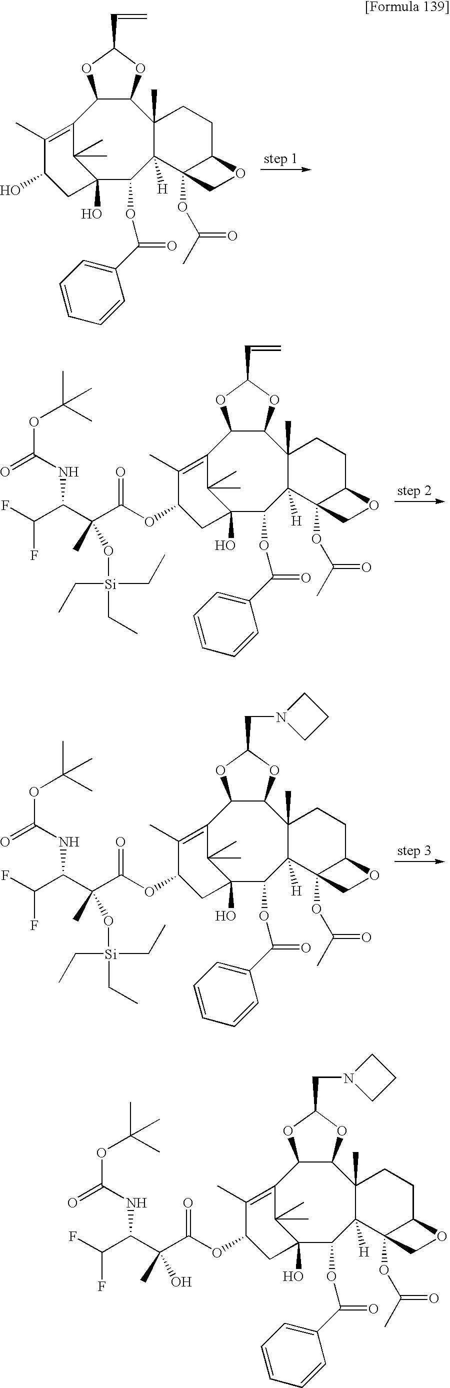

### Step 1: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3R)-3-(tert-butoxycarbonylamino)-4,4-difluoro-2-methyl-2-[(triethylsilyl)oxy]butyrate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-11-ene and the compound obtained in Reference Example 32, Step 4 were used as starting materials to perform the same procedure as that of Example 31, Step 1 and thereby to obtain the title compound as white amorphous solid.

¹H-NMR (400 MHz, CDCl₃) δ: 0.62-0.80 (6H, m), 0.98 (9H, t, J=7.8 Hz), 1.34 (3H, s), 1.36 (9H, s), 1.51 (3H, s), 1.59 (3H, s), 1.64 (3H, s), 1.72-2.20 (7H, m), 1.75 (3H, s), 2.50 (3H, s), 2.89 (1H, d, J=5.1 Hz), 4.15-4.37 (1H, m), 4.18 (1H, d, J=7.3 Hz), 4.25 (1H, d, J=8.3 Hz), 4.30 (1H, d, J=8.3 Hz), 4.90 (1H, s), 4.99 (1H, d, J=10.5 Hz), 5.21 (1H, d, J=6.3 Hz), 5.25 (1H, d, J=7.1 Hz), 5.46 (1H, d, J=10.5 Hz), 5.58 (1H, d, J=17.3 Hz), 5.86 (1H, dt, J=55.9, 5.1 Hz), 5.95-6.05 (2H, m), 6.12 (1H, t, J=9.3 Hz), 7.49 (2H, t, J=7.8 Hz), 7.60 (1H, t, J=7.6 Hz), 8.16 (2H, d, J=7.1 Hz).

FAB-MS; m/z: 934 (M+H)⁺.

### Step 2: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3R)-3-(tert-butoxycarbonylamino)-4,4-difluoro-2-methyl-2-[(triethylsilyl)oxy]butyrate

The compound obtained in Step 1 mentioned above was used as a starting material to perform the same procedure as that of Example 1 and thereby to obtain the title compound as white amorphous solid.

¹H-NMR (400 MHz, CDCl₃) δ: 0.60-0.80 (6H, m), 0.98 (9H, t, J=7.9 Hz), 1.33 (3H, s), 1.37 (9H, s), 1.51 (3H, s), 1.62 (6H, s), 1.70-2.21 (9H, m), 1.73 (3H, s), 2.63 (3H, s), 2.73 (1H, dd, J=12.4, 5.9 Hz), 2.81 (1H, dd, J=12.2, 4.4 Hz), 2.87 (1H, d, J=5.4 Hz), 3.31-3.37 (4H, m), 4.11 (1H, d, J=7.1 Hz), 4.24 (1H, d, J=8.5 Hz), 4.27-4.36 (2H, m), 4.84 (1H, t, J=5.1 Hz), 4.90 (1H, s), 4.98 (1H, d, J=10.7 Hz), 5.16 (1H, d, J=7.3 Hz), 5.86 (1H, dt, J=55.9, 5.9 Hz), 5.94 (1H, d, J=5.9 Hz), 6.11 (1H, t, J=8.3 Hz), 7.49 (2H, t, J=7.7 Hz), 7.61 (1H, t, J=7.3 Hz), 8.16 (2H, d, J=7.1 Hz).

FAB-MS; m/z: 977 (M+H)⁺.

### Step 3: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3R)-3-(tert-butoxycarbonylamino)-4,4-difluoro-2-hydroxy-2-methylbutyrate

The compound obtained in Step 2 mentioned above was used as a starting material to perform the same procedure as that of Example 31, Step 2 and thereby to obtain the title compound as white amorphous solid.

¹H-NMR (400 MHz, CDCl₃) δ: 1.28 (3H, s), 1.41 (9H, s), 1.49 (3H, s), 1.62 (3H, s), 1.63 (3H, s), 1.76 (3H, s), 1.83-2.34 (10H, m), 2.63 (3H, s), 2.74 (1H, dd, J=12.4, 5.9 Hz), 2.82 (1H, dd, J=12.2, 4.4 Hz), 2.94 (1H, d, J=4.9 Hz), 3.31-3.40 (4H, m), 4.11 (1H, d, J=7.1 Hz), 4.27 (1H, d, J=8.5 Hz), 4.30-4.44 (2H, m), 4.87 (1H, t, J=4.6 Hz), 4.91 (1H, brs), 5.38 (1H, d, J=10.7 Hz), 5.20 (1H, d, J=7.3 Hz), 5.99 (1H, d, J=5.1 Hz), 6.08 (1H, dt, J=55.9, 3.9 Hz), 6.19 (1H, t, J=8.3 Hz), 7.49 (2H, t, J=7.7 Hz), 7.61 (1H, t, J=7.3 Hz), 8.15 (2H, d, J=8.1 Hz)

FAB-MS; m/z: 863 (M+H)⁺.

### Example 68

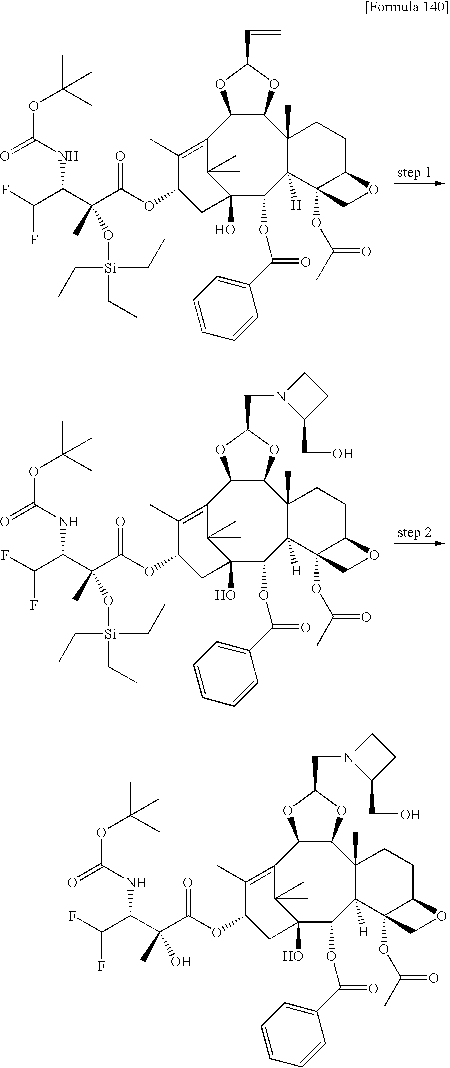

### Step 1: (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-11-en-13-yl

### (2R,3R)-3-(tert-butoxycarbonylamino)-4,4-difluoro-2-methyl-2-[(triethylsilyl)oxy]butyrate

The compound obtained in Example 67, Step 1 and the compound obtained in Reference Example 25 were used as starting materials to perform the same procedure as that of Example 1 and thereby to obtain the title compound as white amorphous solid.

¹H-NMR (400 MHz, CDCl₃) δ: 0.60-0.79 (6H, m), 0.98 (9H, t, J=7.9 Hz), 1.32 (3H, s), 1.36 (9H, s), 1.51 (3H, s), 1.53-2.27 (10H, m), 1.59 (3H, s), 1.63 (3H, s), 1.73 (3H, s), 2.63 (3H, s), 2.81 (1H, dd, J=12.7, 4.6 Hz), 2.85-2.91 (2H, m), 3.06-3.13 (1H, m), 3.38-3.53 (3H, m), 3.63 (1H, dd, J=11.2, 3.2 Hz), 4.12 (1H, d, J=7.1 Hz), 4.24 (1H, d, J=8.5 Hz), 4.27-4.36 (2H, m), 4.85 (1H, t, J=5.1 Hz), 4.90 (1H, s), 4.98 (1H, d, J=10.7 Hz), 5.18 (1H, d, J=7.3 Hz), 5.86 (1H, dt, J=55.9, 5.9 Hz), 5.93 (1H, d, J=5.9 Hz), 6.12 (1H, t, J=8.8 Hz), 7.49 (2H, t, J=7.7 Hz), 7.61 (1H, t, J=7.3 Hz), 8.16 (2H, d, J=7.1 Hz).

FAB-MS; m/z: 1007 (M+H)⁺.

### Step 2: (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-11-en-13-yl

### (2R,3R)-3-(tert-butoxycarbonylamino)-4,4-difluoro-2-hydroxy-2-methylbutyrate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.27 (3H, s), 1.42 (9H, s), 1.49 (3H, s), 1.60 (3H, s), 1.61-2.28 (11H, m), 1.63 (3H, s), 1.76 (3H, s), 2.63 (3H, s), 2.82 (1H, dd, J=12.7, 4.6 Hz), 2.89 (1H, dd, J=12.9, 5.4 Hz), 2.94 (1H, d, J=5.1 Hz), 3.06-3.13 (1H, m), 3.39-3.52 (3H, m), 3.65 (1H, dd, J=11.2, 3.2 Hz), 4.13 (1H, d, J=7.3 Hz), 4.26 (1H, d, J=8.5 Hz), 4.29-4.44 (2H, m), 4.88 (1H, t, J=5.1 Hz), 4.91 (1H, s), 5.34 (1H, d, J=10.7 Hz), 5.22 (1H, d, J=7.3 Hz), 5.98 (1H, d, J=5.9 Hz), 6.07 (1H, dt, J=55.9, 5.9 Hz), 6.19 (1H, t, J=8.8 Hz), 7.49 (2H, t, J=7.7 Hz), 7.61 (1H, t, J=7.3 Hz), 8.14 (2H, d, J=7.8 Hz).

FAB-MS; m/z: 893 (M+H)⁺.

### Example 69

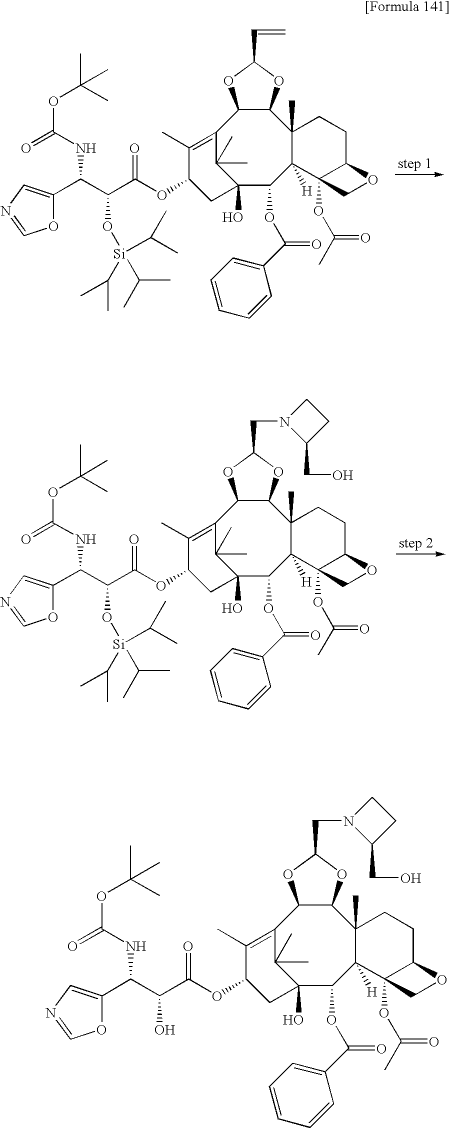

### Step 1: (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-1′-en-13-yl

### (2R,3R)-3-(tert-butoxycarbonylamino)-3-(5-oxazolyl)-2-[(triisopropylsilyl)oxy]propionate

The compound obtained in Example 55, Step 1 and the compound obtained in Reference Example 25 were used as starting materials to perform the same procedure as that of Example 1 and thereby to obtain the title compound as white amorphous solid.

¹H-NMR (400 MHz, CDCl₃) δ: 0.94-1.07 (21H, m), 1.28 (3H, s), 1.40 (9H, s), 1.51 (3H, s), 1.59 (3H, s), 1.71-2.37 (9H, m), 1.78 (3H, s), 2.41 (3H, s), 2.81 (1H, dd, J=12.5, 4.6 Hz), 2.90 (1H, dd, J=12.9, 4.9 Hz), 2.94 (1H, d, J=5.9 Hz), 3.06-3.14 (1H, m), 3.39-3.53 (3H, m), 3.64 (1H, dd, J=12.0, 3.2 Hz), 4.16 (1H, d, J=7.3 Hz), 4.21 (1H, d, J=8.3 Hz), 4.34 (1H, d, J=8.5 Hz), 4.85 (1H, t, J=4.9 Hz), 4.94 (2H, brs), 5.21 (1H, d, J=7.8 Hz), 5.30 (1H, d, J=10.3 Hz), 5.39 (1H, d, J=10.0 Hz), 5.96 (1H, d, J=5.4 Hz), 6.07 (1H, t, J=8.5 Hz), 7.02 (1H, s), 7.48 (2H, t, J=7.6 Hz), 7.60 (1H, t, J=7.8 Hz), 7.87 (1H, s), 8.14 (2H, d, J=8.1 Hz).

FAB-MS; m/z: 1052 (M+H)⁺.

### Step 2: (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-11-en-13-yl

### (2R,3R)-3-(tert-butoxycarbonylamino)-2-hydroxy-3-(5-oxazolyl)propionate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.26 (3H, s), 1.41 (9H, s), 1.47 (3H, s), 1.58 (3H, s), 1.64 (3H, s), 1.76-2.43 (11H, m), 2.27 (3H, s), 2.83 (1H, dd, J=12.7, 4.6 Hz), 2.86-2.92 (2H, m), 3.06-3.14 (1H, m), 3.39-3.53 (3H, m), 3.66 (1H, dd, J=12.0, 2.9 Hz), 4.10 (1H, d, J=7.3 Hz), 4.24 (1H, d, J=8.5 Hz), 4.31 (1H, d, J=7.8 Hz), 4.63 (1H, s), 4.88 (1H, t, J=4.9 Hz), 4.91 (1H, s), 5.21 (1H, d, J=6.8 Hz), 5.31 (1H, d, J=9.0 Hz), 5.70 (1H, d, J=9.0 Hz), 5.98 (1H, d, J=4.9 Hz), 6.06 (1H, t, J=9.3 Hz), 7.07 (1H, s), 7.48 (2H, t, J=7.7 Hz), 7.61 (1H, t, J=7.6 Hz), 7.85 (1H, s), 8.13 (2H, d, J=8.1 Hz).

FAB-MS; m/z: 896 (M+H)⁺.

### Example 70

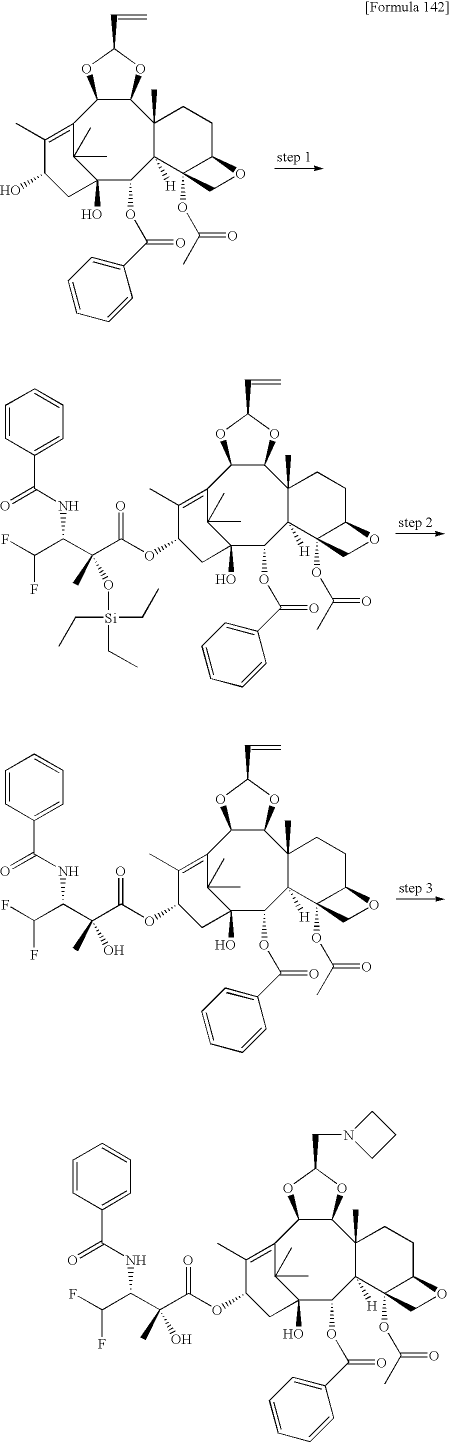

### Step 1: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3R)-3-(benzoylamino)-4,4-difluoro-2-methyl-2-[(triethylsilyl)oxy]butyrate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-11-ene and the compound obtained in Reference Example 33 were used as starting materials to perform the same procedure as that of Example 31, Step 1 and thereby to obtain the title compound as white amorphous solid, and used as it was for the following reaction.

### Step 2: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3R)-3-(benzoylamino)-4,4-difluoro-2-hydroxy-2-methylbutyrate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.27 (3H, s), 1.49 (3H, s), 1.64 (3H, s), 1.67 (3H, s), 1.75 (3H, s), 1.81-2.30 (8H, m), 2.45 (3H, s), 2.97 (1H, d, J=4.9 Hz), 4.17 (1H, d, J=7.1 Hz), 4.28 (1H, d, J=8.8 Hz), 4.34 (1H, d, J=8.5 Hz), 4.86 (1H, s), 4.93 (1H, s), 4.95-5.05 (1H, m), 5.24 (1H, d, J=6.3 Hz), 5.27 (1H, d, J=7.3 Hz), 5.47 (1H, d, J=10.5 Hz), 5.58 (1H, d, J=17.3 Hz), 5.95-6.06 (2H, m), 6.23 (1H, t, J=8.8 Hz), 6.30 (1H, dt, J=55.9, 2.7 Hz), 7.43-7.65 (6H, m), 7.84 (2H, d, J=8.1 Hz), 8.15 (2H, d, J=8.1 Hz).

FAB-MS; m/z: 824 (M+H)⁺.

### Step 3: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3R)-3-(benzoylamino)-4,4-difluoro-2-hydroxy-2-methylbutyrate

The compound obtained in Step 2 mentioned above was used as a starting material to perform the same procedure as that of Example 1 and thereby to obtain the title compound as white amorphous solid.

¹H-NMR (400 MHz, CDCl₃) δ: 1.26 (3H, s), 1.48 (3H, s), 1.61 (3H, s), 1.67 (3H, s), 1.73 (3H, s), 1.79-2.27 (10H, m), 2.44 (3H, s), 2.74 (1H, dd, J=12.4, 5.9 Hz), 2.82 (1H, dd, J=12.2, 4.4 Hz), 2.95 (1H, d, J=4.9 Hz), 3.31-3.40 (4H, m), 4.10 (1H, d, J=7.1 Hz), 4.28 (1H, d, J=8.5 Hz), 4.34 (1H, d, J=8.5 Hz), 4.79-4.89 (1H, m), 4.86 (1H, t, J=4.6 Hz), 4.93 (1H, brs), 4.95-5.06 (1H, m), 5.18 (1H, d, J=7.3 Hz), 6.01 (1H, d, J=5.1 Hz), 6.22 (1H, t, J=8.3 Hz), 6.29 (1H, dt, J=55.9, 3.9 Hz), 7.42-7.57 (7H, m), 7.62 (1H, t, J=7.3 Hz), 8.15 (2H, d, J=8.1 Hz).

FAB-MS; m/z: 867 (M+H)⁺.

### Example 71

### (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-11-en-13-yl

### (2R,3R)-3-(benzoylamino)-4,4-difluoro-2-hydroxy-2-methylbutyrate

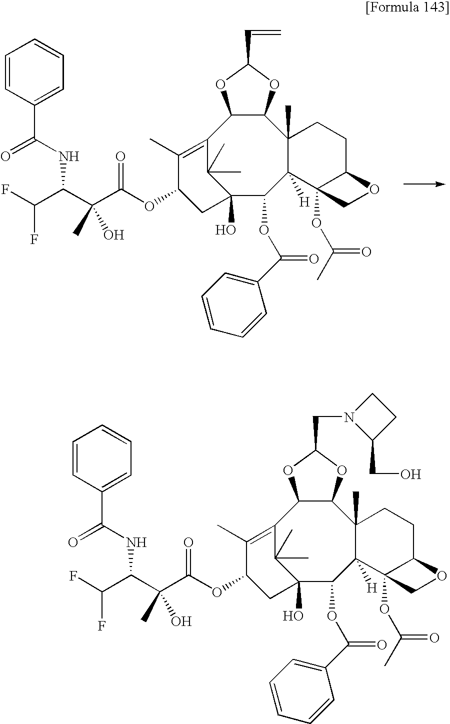

The compound obtained in Example 70, Step 2 and the compound obtained in Reference Example 25 were used as starting materials to perform the same procedure as that of Example 1 and thereby to obtain the title compound as white amorphous solid.

¹H-NMR (400 MHz, CDCl₃) δ: 1.25 (3H, s), 1.48 (3H, s), 1.59 (3H, s), 1.67 (3H, s), 1.73 (3H, s), 1.78-2.31 (11H, m), 2.45 (3H, s), 2.82 (1H, dd, J=12.9, 4.9 Hz), 2.90 (1H, dd, J=12.9, 5.1 Hz), 2.95 (1H, d, J=4.9 Hz), 3.06-3.14 (1H, m), 3.39-3.54 (3H, m), 3.64 (1H, dd, J=11.5, 2.7 Hz), 4.11 (1H, d, J=7.1 Hz), 4.27 (1H, d, J=8.5 Hz), 4.33 (1H, d, J=8.5 Hz), 4.73-4.85 (1H, m), 4.88 (1H, t, J=4.6 Hz), 4.93 (1H, brs), 4.95-5.05 (1H, m), 5.20 (1H, d, J=7.3 Hz), 5.99 (1H, d, J=5.1 Hz), 6.21 (1H, t, J=8.3 Hz), 6.29 (1H, dt, J=55.9, 2.4 Hz), 7.43-7.57 (7H, m), 7.62 (1H, t, J=7.3 Hz), 8.15 (2H, d, J=8.1 Hz).

FAB-MS; m/z: 897 (M+H)⁺.

### Example 72

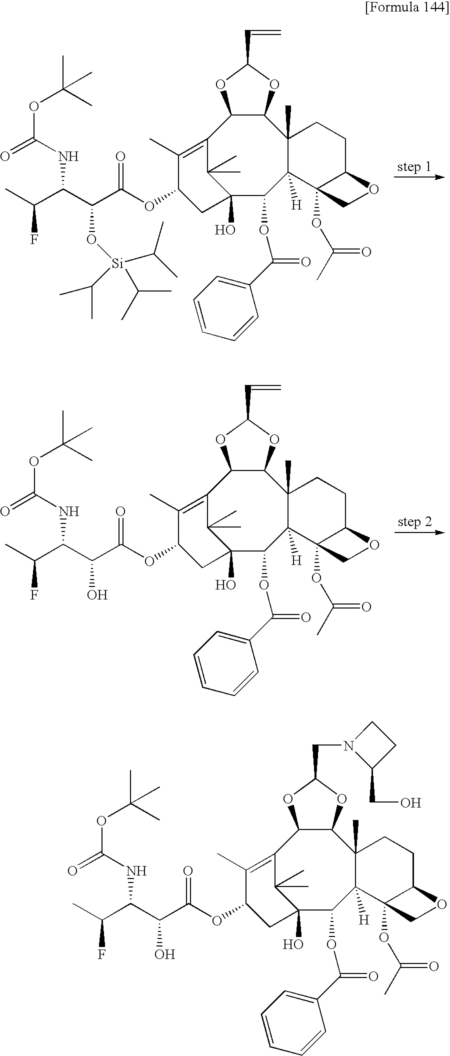

### Step 1: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3R,4S)-3-(tert-butoxycarbonylamino)-4-fluoro-2-hydroxypentanoate

The compound obtained in Example 66, Step 1 was used as a starting material to perform the same procedure as that of Example 31, Step 2 and thereby to obtain the title compound as white amorphous solid.

¹H-NMR (400 MHz, CDCl₃) δ: 1.24-2.10 (10H, m), 1.26 (3H, s), 1.40 (9H, s), 1.56 (3H, s), 1.64 (3H, s), 1.80 (3H, s), 2.33-2.39 (1H, m), 2.35 (3H, s), 2.94 (1H, d, J=4.9 Hz), 4.18-4.25 (3H, m), 4.34 (1H, d, J=8.3 Hz), 4.60-4.79 (2H, m), 4.94-4.97 (2H, m), 5.25 (1H, d, J=6.1 Hz), 5.31 (1H, d, J=7.1 Hz), 5.46 (1H, d, J=10.5 Hz), 5.58 (1H, d, J=17.1 Hz), 5.97-6.05 (2H, m), 6.10 (1H, t, J=8.2 Hz), 7.48 (2H, t, J=7.8 Hz), 7.61 (1H, t, J=7.3 Hz), 8.12-8.15 (2H, m).

FAB-MS; m/z: 802 (M+H)⁺, 824 (M+Na)⁺.

### Step 2: (1S,2S,3R,4S,5R,8R,9S,10R,13S)-4-(Acetoxy)-2-(benzoyloxy)-5,20-epoxy-1-hydroxy-9,10-{(1 S)-2-[(2S)-2-hydroxymethylazetidino]ethylidenedioxy}tax-11-en-13-yl

### (2R,3R,4S)-3-(tert-butoxycarbonylamino)-4-fluoro-2-hydroxypentanoate

The compound obtained in Step 1 mentioned above and the compound obtained in Reference Example 25 were used as starting materials to perform the same procedure as that of Example 1 and thereby to obtain the title compound as white amorphous solid.

¹H-NMR (400 MHz, CDCl₃) δ: 1.23-2.38 (14H, m), 1.24 (3H, s), 1.40 (9H, s), 1.49 (3H, s), 1.59 (3H, s), 1.77 (3H, s), 2.34 (3H, s), 2.83 (1H, dd, J=12.7, 4.6 Hz), 2.88-2.93 (2H, m), 3.11 (1H, dd, J=16.4, 8.5 Hz), 3.43 (1H, dd, J=11.5, 2.4 Hz), 3.47-3.53 (2H, m), 3.66 (1H, dd, J=11.5, 2.7 Hz), 4.13 (1H, d, J=7.1 Hz), 4.18-4.24 (2H, m), 4.33 (1H, d, J=8.5 Hz), 4.59-4.78 (2H, m), 4.89 (1H, t, J=4.6 Hz), 4.93 (1H, s), 4.99 (1H, d, J=10.3 Hz), 5.24 (1H, d, J=7.1 Hz), 5.99 (1H, d, J=5.1 Hz), 6.08 (1H, t, J=8.5 Hz), 7.49 (2H, t, J=7.7 Hz), 7.61 (1H, t, J=7.3 Hz), 8.13 (2H, d, J=7.3 Hz).

FAB-MS; m/z: 875 (M+H)⁺.

### Example 73

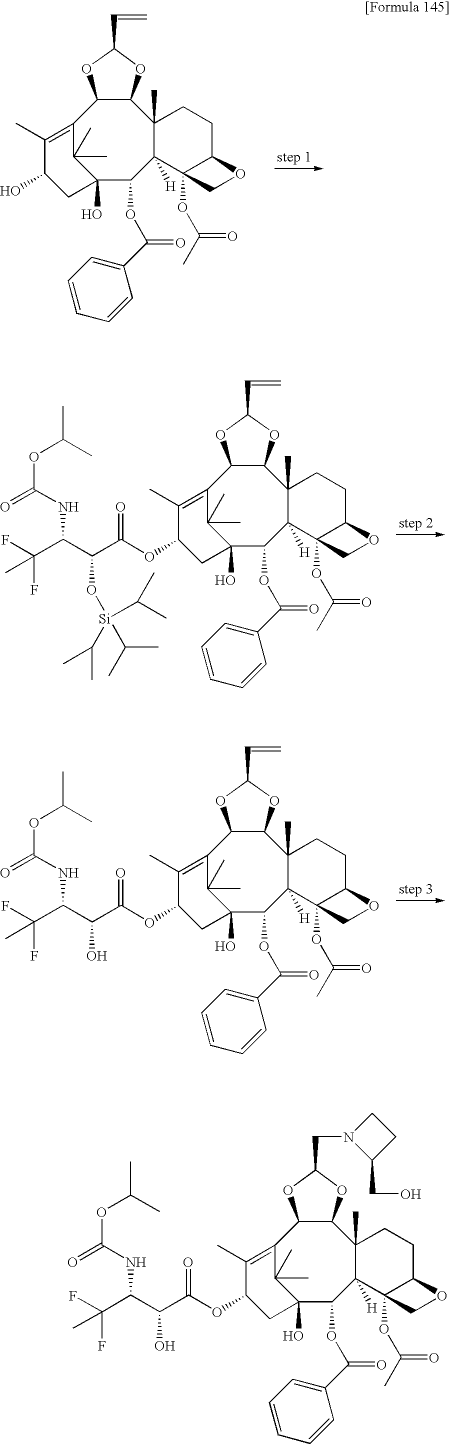

### Step 1: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3R)-4,4-difluoro-3-(isopropoxycarbonylamino)-2-[(triisopropylsilyl)oxy]pentanoate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-11-ene and the compound obtained in Reference Example 34 were used as starting materials to perform the same procedure as that of Example 31, Step 1 and thereby to obtain the title compound as white amorphous solid.

¹H-NMR (400 MHz, CDCl₃) δ: 0.96-2.32 (13H, m), 1.07-1.15 (21H, m), 1.31 (3H, s), 1.51 (3H, s), 1.64 (3H, s), 1.69 (3H, t, J=18.6 Hz), 1.79 (3H, s), 2.31 (3H, s), 2.93 (1H, d, J=4.9 Hz), 4.20 (2H, d, J=7.1 Hz), 4.26-4.37 (2H, m), 4.77-4.85 (1H, m), 4.93 (1H, s), 5.03 (1H, s), 5.18-5.28 (3H, m), 5.45 (1H, d, J=10.5 Hz), 5.57 (1H, d, J=17.2 Hz), 5.94-6.05 (3H, m), 7.49 (2H, t, J=7.7 Hz), 7.58-7.63 (1H, m), 8.12-8.17 (2H, m).

### Step 2: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3R)-4,4-difluoro-2-hydroxy-3-(isopropoxycarbonylamino)pentanoate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.13-2.42 (11H, m), 1.25 (3H, s), 1.49 (3H, s), 1.64 (3H, s), 1.72 (3H, t, J=18.8 Hz), 1.76 (3H, s), 2.35 (3H, s), 2.94 (1H, d, J=4.9 Hz), 4.17 (1H, d, J=6.8 Hz), 4.25 (1H, d, J=8.5 Hz), 4.34 (1H, d, J=8.5 Hz), 4.59-4.72 (2H, m), 4.78 (1H, s), 4.88-4.98 (2H, m), 5.25 (1H, d, J=6.1 Hz), 5.30 (1H, d, J=6.8 Hz), 5.42 (1H, d, J=10.2 Hz), 5.47 (1H, d, J=10.5 Hz), 5.58 (1H, d, J=17.1 Hz), 5.96-6.06 (2H, m), 6.13-6.20 (3H, m), 7.48 (2H, t, J=7.7 Hz), 7.59-7.64 (1H, m), 8.11-8.16 (2H, m).

### Step 3: (1S,2S,3R,4S,5R,8R,9S,10R,13S)-4-(Acetoxy)-2-(benzoyloxy-5,20-epoxy-1-hydroxy-9,10-{(1S)-2-[(2S)-2-hydroxymethylazetidino]ethylidenedioxy}tax-11-en-13-yl

### (2R,3R)-4,4-difluoro-2-hydroxy-3-(isopropoxycarbonylamino)pentanoate

The compound obtained in Step 2 mentioned above and the compound obtained in Reference Example 25 were used as starting materials to perform the same procedure as that of Example 1 and thereby to obtain the title compound as white amorphous solid.

¹H-NMR (400 MHz, CDCl₃) δ: 1.12-2.44 (17H, m), 1.23 (3H, s), 1.48 (3H, s), 1.58 (3H, s), 1.72 (3H, t, J=18.8 Hz), 1.74 (3H, s), 2.34 (3H, s), 2.84 (1H, dd, J=12.7, 4.4 Hz), 2.87-2.94 (2H, m), 3.11 (1H, q, J=8.2 Hz), 3.40-3.46 (1H, m), 3.46-3.55 (2H, m), 3.62-3.69 (1H, m), 4.12 (1H, d, J=7.3 Hz), 4.24 (1H, d, J=8.3 Hz), 4.33 (1H, d, J=8.3 Hz), 4.58-4.67 (1H, m), 4.77 (1H, s), 4.86-4.95 (3H, m), 5.22 (1H, d, J=7.1 Hz), 5.43 (1H, d, J=10.5 Hz), 5.99 (1H, d, J=5.1 Hz), 6.14 (1H, t, J=8.3 Hz), 7.48 (2H, t, J=7.7 Hz), 7.58-7.64 (1H, m), 8.11-8.15 (2H, m).

FAB-MS; m/z: 879 (M+H)⁺.

### Example 74

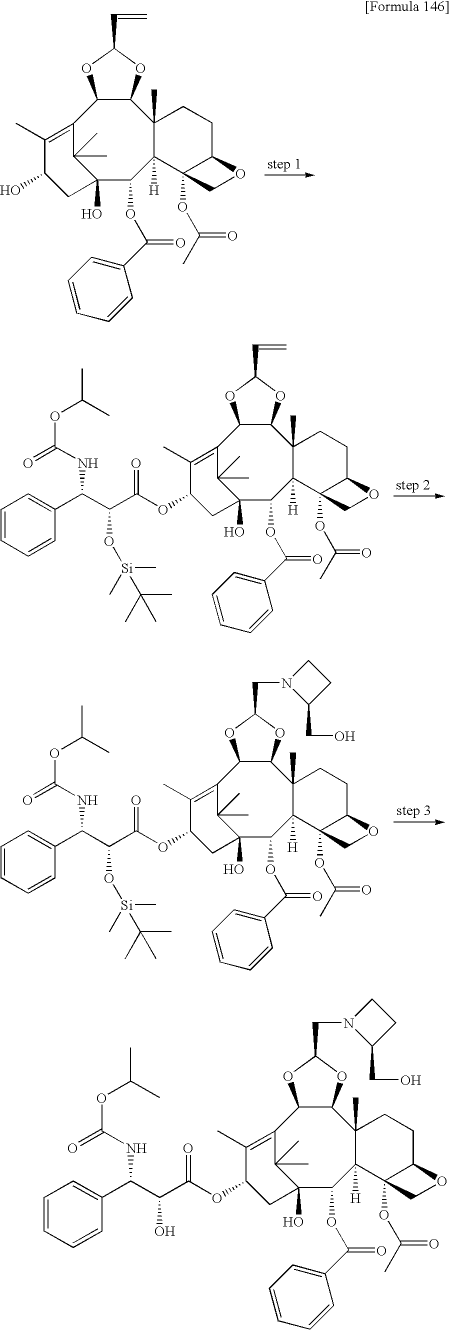

### Step 1: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3S)-2-[(tert-butyldimethylsilyl)oxy]-3-(isopropoxycarbonylamino)-3-phenylpropionate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-11-ene and the compound obtained in Reference Example 35 were used as starting materials to perform the same procedure as that of Example 31, Step 1 and thereby to obtain the title compound as white amorphous solid.

¹H-NMR (400 MHz, CDCl₃) δ: −0.35 (3H, s), −0.09 (3H, s), 0.74 (9H, s), 1.16-1.23 (6H, m), 1.35 (3H, s), 1.52 (3H, s), 1.66 (3H, s), 1.77 (3H, s), 1.83-2.31 (7H, m), 2.50 (3H, s), 2.94 (1H, d, J=5.1 Hz), 4.21 (1H, d, J=7.6 Hz), 4.24 (1H, d, J=8.8 Hz), 4.35 (1H, d, J=8.8 Hz), 4.58 (1H, s), 4.79-4.88 (1H, m), 4.98 (1H, s), 5.22 (1H, d, J=6.1 Hz), 5.27 (1H, d, J=7.1 Hz), 5.37 (1H, brs), 5.46 (1H, d, J=10.2 Hz), 5.52-5.58 (1H, m), 5.58 (1H, d, J=17.3 Hz), 5.96-6.05 (2H, m), 6.24 (1H, t, J=9.0 Hz), 7.22-7.52 (7H, m), 7.60 (1H, t, J=7.6 Hz), 8.15 (2H, d, J=7.3 Hz).

FAB-MS; m/z: 932 (M+H)⁺.

### Step 2: (1S,2S,3R,4S,5R,8R,9S,10R,13S)-4-(Acetoxy)-2-(benzoyloxy)-5,20-epoxy-1-hydroxy-9,10-{(1 S)-2-[(2S)-2-hydroxymethylazetidino]ethylidenedioxy}tax-11-en-13-yl

### (2R,3S)-2-[(tert-butyldimethylsilyl)oxy]-3-(isopropoxycarbonylamino)-3-phenylpropionate

The compound obtained in Step 1 mentioned above and the compound obtained in Reference Example 25 were used as starting materials to perform the same procedure as that of Example 1 and thereby to obtain the title compound as white amorphous solid.

¹H-NMR (400 MHz, CDCl₃) δ: −0.35 (3H, s), −0.09 (3H, s), 0.74 (9H, s), 1.16-1.22 (6H, m), 1.33 (3H, s), 1.51 (3H, s), 1.60 (3H, s), 1.75 (3H, s), 1.83-2.32 (10H, m), 2.50 (3H, s), 2.81 (1H, dd, J=12.9, 4.9 Hz), 2.90 (1H, dd, J=12.7, 5.1 Hz), 2.93 (1H, d, J=5.1 Hz), 3.06-3.14 (1H, m), 3.39-3.53 (3H, m), 3.63 (1H, dd, J=11.5, 3.2 Hz), 4.16 (1H, d, J=7.3 Hz), 4.24 (1H, d, J=8.5 Hz), 4.34 (1H, d, J=8.8 Hz), 4.57 (1H, s), 4.79-4.88 (2H, m), 4.98 (1H, s), 5.19 (1H, d, J=7.3 Hz), 5.35 (1H, d, J=8.5 Hz), 5.55 (1H, d, J=9.5 Hz), 5.97 (1H, d, J=5.4 Hz), 6.23 (1H, t, J=9.0 Hz), 7.25-7.41 (5H, m), 7.48 (2H, t, J=7.6 Hz), 7.60 (1H, t, J=7.6 Hz), 8.15 (2H, d, J=7.3 Hz).

FAB-MS; m/z: 1005 (M+H)⁺.

### Step 3: (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-11-en-13-yl

### (2R,3S)-2-hydroxy-3-(isopropoxycarbonylamino)-3-phenylpropionate

The compound obtained in Step 2 mentioned above was used as a starting material to perform the same procedure as that of Example 31, Step 2 and thereby to obtain the title compound as white amorphous solid.

¹H-NMR (400 MHz, CDCl₃) δ: 1.18-1.21 (6H, m), 1.23 (3H, s), 1.47 (3H, s), 1.58 (3H, s), 1.63 (3H, s), 1.70-2.40 (11H, m), 2.27 (3H, s), 2.82 (1H, dd, J=12.9, 4.9 Hz), 2.85-2.91 (2H, m), 3.05-3.13 (1H, m), 3.39-3.53 (3H, m), 3.64 (1H, dd, J=11.5, 3.2 Hz), 4.09 (1H, d, J=7.3 Hz), 4.23 (1H, d, J=8.5 Hz), 4.31 (1H, d, J=8.8 Hz), 4.63 (1H, s), 4.82-4.90 (2H, m), 4.91 (1H, s), 5.20 (1H, d, J=7.3 Hz), 5.36 (1H, d, J=8.5 Hz), 5.81 (1H, d, J=9.5 Hz), 5.98 (1H, d, J=5.1 Hz), 6.08 (1H, t, J=9.0 Hz), 7.25-7.49 (7H, m), 7.60 (1H, t, J=7.6 Hz), 8.11 (2H, d, J=7.3 Hz).

FAB-MS; m/z: 891 (M+H)⁺.

### Example 75

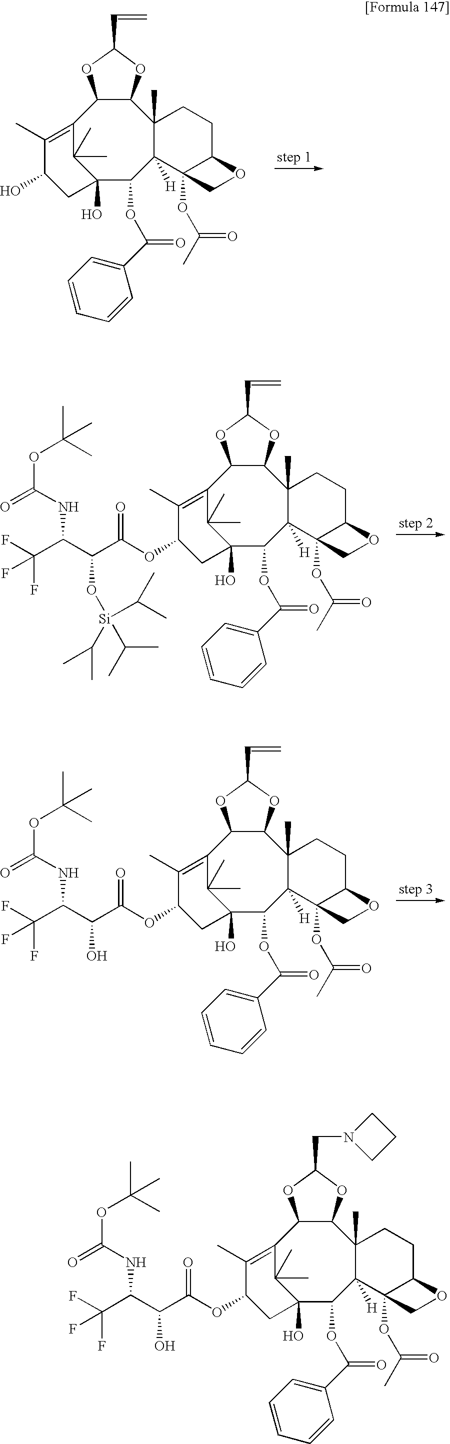

### Step 1: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3R)-3-(tert-butoxycarbonylamino)-4,4,4-trifluoro-2-[(triisopropylsilyl)oxy]butyrate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-11-ene and (3R,4R)-1-(tert-butoxycarbonyl)-4-(trifluoromethyl)-3-[(triisopropylsilyl)oxy]-2-azetidinone (J. Fluorine. Chem., 2004, 125, 487) were used as starting materials to perform the same procedure as that of Example 31, Step 1 and thereby to obtain the title compound as white amorphous solid.

¹H-NMR (400 MHz, CDCl₃) δ: 1.05-2.33 (8H, m), 1.07-1.15 (21H, m), 1.29 (3H, s), 1.39 (3H, s), 1.53 (9H, s), 1.64 (3H, s), 1.78 (3H, s), 2.32 (3H, s), 2.93 (1H, d, J=5.1 Hz), 4.18-4.23 (2H, m), 4.34 (1H, d, J=9.0 Hz), 4.61-4.69 (1H, m), 4.88-4.94 (1H, m), 4.96 (1H, s), 5.14-5.22 (2H, m), 5.26 (1H, d, J=7.1 Hz), 5.45 (1H, d, J=10.5 Hz), 5.57 (1H, d, J=17.3 Hz), 5.95-6.05 (2H, m), 7.50 (2H, t, J=7.8 Hz), 7.58-7.64 (1H, m), 8.13-8.17 (2H, m).

### Step 2: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3R)-3-(tert-butoxycarbonylamino)-2-hydroxy-4,4,4-trifluorobutyrate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.23-2.39 (7H, m), 1.25 (3H, s), 1.43 (9H, s), 1.55 (3H, s), 1.64 (3H, s), 1.77 (3H, s), 2.34 (3H, s), 2.94 (1H, d, J=4.9 Hz), 4.17 (1H, d, J=7.1 Hz), 4.26 (1H, d, J=8.5 Hz), 4.34 (1H, d, J=8.3 Hz), 4.72 (1H, s), 4.82 (1H, s), 4.87-4.95 (2H, m), 5.26 (1H, d, J=6.1 Hz), 5.31 (1H, d, J=6.6 Hz), 5.36 (1H, d, J=10.5 Hz), 5.47 (1H, d, J=10.5 Hz), 5.58 (1H, d, J=17.3 Hz), 5.95-6.06 (2H, m), 6.13-6.20 (1H, m), 7.48 (2H, t, J=7.8 Hz), 7.59-7.64 (1H, m), 8.11-8.15 (2H, m).

### Step 3: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3R)-3-(tert-butoxycarbonylamino)-2-hydroxy-4,4,4-trifluorobutyrate

The compound obtained in Step 2 mentioned above was used as a starting material to perform the same procedure as that of Example 1 and thereby to obtain the title compound as white amorphous solid.

¹H-NMR (400 MHz, CDCl₃) δ: 1.22-2.37 (10H, m), 1.23 (3H, s), 1.42 (9H, s), 1.49 (3H, s), 1.61 (3H, s), 1.75 (3H, s), 2.34 (3H, s), 2.75 (1H, dd, J=12.6, 6.0 Hz), 2.84 (1H, dd, J=12.4, 4.1 Hz), 2.92 (1H, d, J=4.9 Hz), 3.33-3.41 (4H, m), 4.11 (1H, d, J=7.1 Hz), 4.25 (1H, d, J=8.5 Hz), 4.34 (1H, d, J=8.0 Hz), 4.72 (1H, s), 4.84-4.94 (3H, m), 5.22 (1H, d, J=7.1 Hz), 5.39 (1H, d, J=10.5 Hz), 6.01 (1H, d, J=4.9 Hz), 6.10-6.18 (1H, m), 7.49 (2H, t, J=7.7 Hz), 7.59-7.64 (1H, m), 8.11-8.15 (2H, m).

FAB-MS; m/z: 867 (M+H)⁺.

### Example 76

### (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-11-en-13-yl

### (2R,3R)-3-(tert-butoxycarbonylamino)-2-hydroxy-4,4,4-trifluorobutyrate

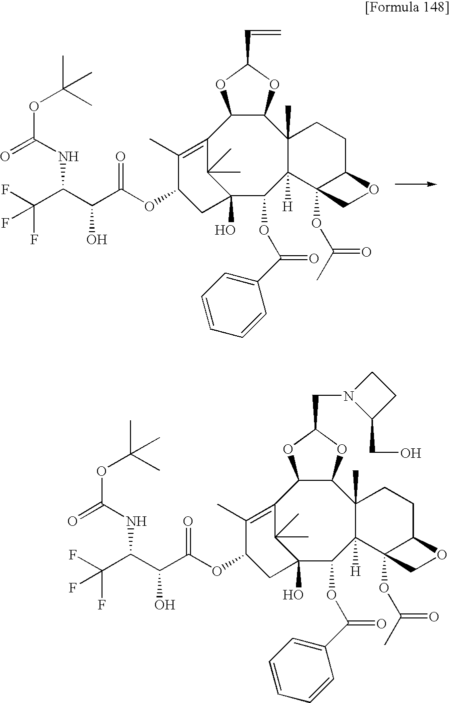

The compound obtained in Example 75, Step 2 and the compound obtained in Reference Example 25 were used as starting materials to perform the same procedure as that of Example 1 and thereby to obtain the title compound as white amorphous solid.

¹H-NMR (400 MHz, CDCl₃) δ: 1.22 (3H, s), 1.22-2.37 (11H, m), 1.43 (9H, s), 1.48 (3H, s), 1.59 (3H, s), 1.75 (3H, s), 2.34 (3H, s), 2.84 (1H, dd, J=12.9, 4.4 Hz), 2.88-2.94 (2H, m), 3.06-3.16 (1H, m), 3.43 (1H, dd, J=11.5, 2.4 Hz), 3.46-3.54 (2H, m), 3.63-3.69 (1H, m), 4.12 (1H, d, J=6.8 Hz), 4.25 (1H, d, J=8.5 Hz), 4.34 (1H, d, J=8.3 Hz), 4.72 (1H, s), 4.85-4.95 (3H, m), 5.23 (1H, d, J=7.1 Hz), 5.38 (1H, d, J=10.7 Hz), 6.00 (1H, d, J=4.9 Hz), 6.09-6.18 (1H, m), 7.49 (2H, t, J=7.7 Hz), 7.58-7.64 (1H, m), 8.11-8.15 (2H, m).

FAB-MS; m/z: 897 (M+H)⁺.

### Example 77

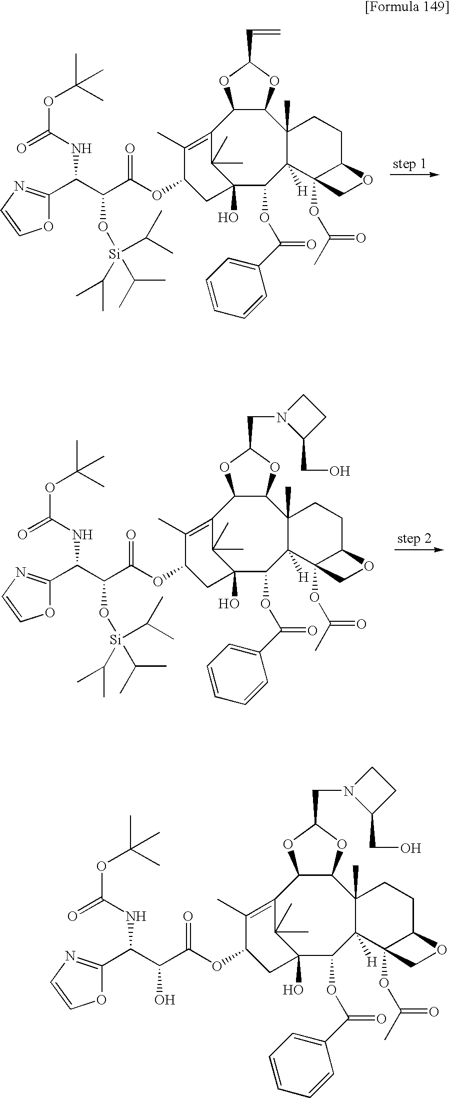

### Step 1: (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-11-en-13-yl

### (2R,3R)-3-(tert-butoxycarbonylamino)-3-(2-oxazolyl)-2-[(triisopropylsilyl)oxy]propionate

The compound obtained in Example 41, Step 1 and the compound obtained in Reference Example 25 were used as starting materials to perform the same procedure as that of Example 1 and thereby to obtain the title compound as white amorphous solid.

¹H-NMR (400 MHz, CDCl₃) δ: 0.94-1.01 (21H, m), 1.29 (3H, s), 1.39 (9H, s), 1.51 (3H, s), 1.59 (3H, s), 1.74-2.39 (10H, m), 1.78 (3H, s), 2.46 (3H, s), 2.81 (1H, dd, J=12.5, 4.6 Hz), 2.89 (1H, dd, J=12.9, 4.9 Hz), 2.95 (1H, d, J=5.9 Hz), 3.06-3.14 (1H, m), 3.39-3.53 (3H, m), 3.63 (1H, dd, J=11.2, 3.2 Hz), 4.16 (1H, d, J=7.3 Hz), 4.21 (1H, d, J=8.3 Hz), 4.34 (1H, d, J=8.5 Hz), 4.85 (1H, t, J=4.9 Hz), 4.94 (2H, brs), 5.20 (1H, d, J=6.8 Hz), 5.44 (1H, d, J=10.3 Hz), 5.53 (1H, d, J=10.0 Hz), 5.96 (1H, d, J=5.4 Hz), 6.08 (1H, t, J=8.5 Hz), 7.11 (1H, s), 7.47 (2H, t, J=7.6 Hz), 7.59 (1H, t, J=7.8 Hz), 7.64 (1H, s), 8.14 (2H, d, J=8.1 Hz).

FAB-MS; m/z: 1052 (M+H)⁺.

### Step 2: (1S,2S,3R,4S,5R,8R,9S,10R,13S)-4-(Acetoxy)-2-(benzoyloxy)-5,20-epoxy-1-hydroxy-9,10-{(11S)-2-[(2S)-2-hydroxymethylazetidino]ethylidenedioxy}tax-11-en-13-yl

### (2R,3R)-3-(tert-butoxycarbonylamino)-2-hydroxy-3-(2-oxazolyl)propionate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.26 (3H, s), 1.43 (9H, s), 1.49 (3H, s), 1.59 (3H, s), 1.74-2.43 (11H, m), 1.79 (3H, s), 2.34 (3H, s), 2.82 (1H, dd, J=12.5, 4.6 Hz), 2.89 (1H, dd, J=12.9, 4.9 Hz), 2.93 (1H, d, J=5.9 Hz), 3.06-3.14 (1H, m), 3.39-3.53 (3H, m), 3.65 (1H, dd, J=11.0, 3.2 Hz), 4.13 (1H, d, J=7.3 Hz), 4.23 (1H, d, J=8.3 Hz), 4.33 (1H, d, J=8.5 Hz), 4.84 (1H, d, J=2.4 Hz), 4.88 (1H, t, J=4.9 Hz), 4.92 (1H, brs), 5.24 (1H, d, J=6.8 Hz), 5.49 (1H, d, J=10.3 Hz), 5.71 (1H, d, J=10.0 Hz), 5.99 (1H, d, J=5.4 Hz), 6.12 (1H, t, J=8.5 Hz), 7.11 (1H, s), 7.47 (2H, t, J=7.6 Hz), 7.60 (1H, t, J=7.3 Hz), 7.67 (1H, s), 8.13 (2H, d, J=8.1 Hz).

FAB-MS; m/z: 896 (M+H)⁺.

### Example 78

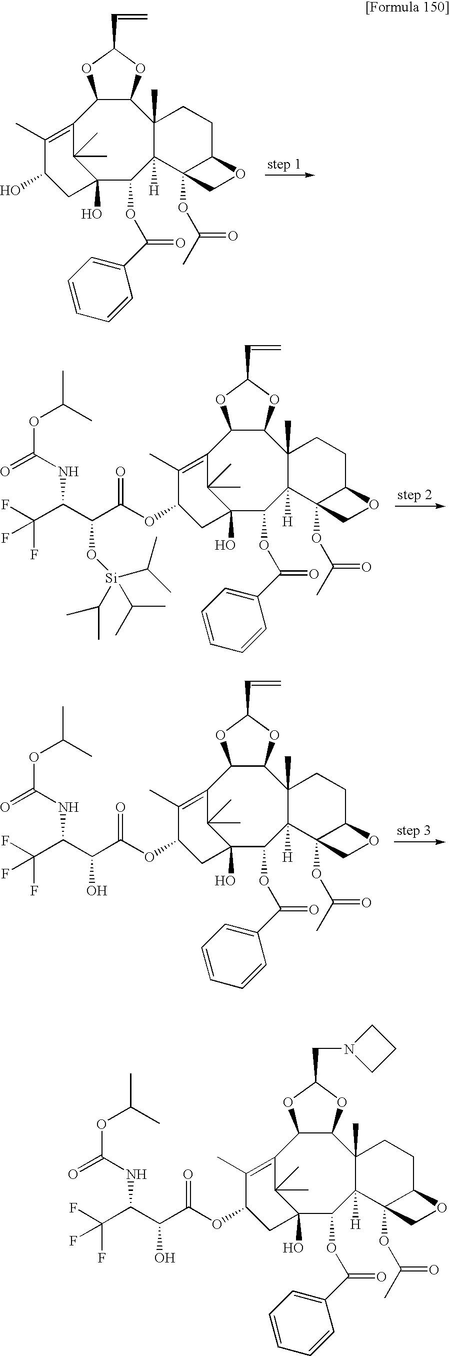

### Step 1: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3R)-3-(isopropoxycarbonylamino)-4,4,4-trifluoro-2-[(triisopropylsilyl)oxy]butyrate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-11-ene and the compound obtained in Reference Example 36 were used as starting materials to perform the same procedure as that of Example 31, Step 1 and thereby to obtain the title compound as white amorphous solid.

¹H-NMR (400 MHz, CDCl₃) δ: 1.04-1.16 (21H, m), 1.05-2.33 (7H, m), 1.18 (3H, d, J=6.1 Hz), 1.22 (3H, d, J=6.4 Hz), 1.31 (3H, s), 1.51 (3H, s), 1.65 (3H, s), 1.78 (3H, s), 2.32 (3H, s), 2.92 (1H, d, J=5.1 Hz), 4.18-4.23 (2H, m), 4.34 (1H, d, J=8.3 Hz), 4.65-4.75 (1H, m), 4.79-4.87 (1H, m), 4.92 (1H, s), 4.96-4.98 (1H, m), 5.20 (1H, d, J=6.1 Hz), 5.23-5.28 (2H, m), 5.46 (1H, d, J=10.8 Hz), 5.57 (1H, d, J=17.4 Hz), 5.95-6.09 (3H, m), 7.50 (2H, t, J=7.8 Hz), 7.58-7.64 (1H, m), 8.13-8.17 (2H, m).

### Step 2: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3R)-2-hydroxy-3-(isopropoxycarbonylamino)-4,4,4-trifluorobutyrate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.16-2.37 (12H, m), 1.24 (3H, s), 1.55 (3H, s), 1.64 (3H, s), 1.75 (3H, s), 2.35 (3H, s), 2.94 (1H, d, J=5.4 Hz), 4.16 (1H, d, J=6.9 Hz), 4.26 (1H, d, J=8.6 Hz), 4.34 (1H, d, J=8.3 Hz), 4.74 (1H, s), 4.90-5.05 (3H, m), 5.25 (1H, d, J=6.1 Hz), 5.30 (1H, d, J=6.9 Hz), 5.43-5.49 (2H, m), 5.58 (1H, d, J=17.4 Hz), 5.96-6.06 (2H, m), 6.14-6.22 (3H, m), 7.48 (2H, t, J=7.7 Hz), 7.59-7.64 (1H, m), 8.11-8.15 (2H, m).

### Step 3: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3S)-2-hydroxy-3-(isopropoxycarbonylamino)-4,4,4-trifluorobutyrate

The compound obtained in Step 2 mentioned above was used as a starting material to perform the same procedure as that of Example 1 and thereby to obtain the title compound as white amorphous solid.

¹H-NMR (400 MHz, CDCl₃) δ: 1.17-2.37 (10H, m), 1.19 (3H, d, J=6.4 Hz), 1.23 (3H, s), 1.25 (3H, d, J=6.1 Hz), 1.48 (3H, s), 1.61 (3H, s), 1.72 (3H, s), 2.34 (3H, s), 2.75 (1H, dd, J=12.5, 5.9 Hz), 2.84 (1H, dd, J=12.5, 4.2 Hz), 2.92 (1H, d, J=4.9 Hz), 3.32-3.41 (4H, m), 4.10 (1H, d, J=7.1 Hz), 4.25 (1H, d, J=8.3 Hz), 4.34 (1H, d, J=8.6 Hz), 4.73 (1H, s), 4.85-5.00 (4H, m), 5.21 (1H, d, J=7.1 Hz), 5.50 (1H, d, J=10.3 Hz), 6.01 (1H, d, J=4.9 Hz), 6.13-6.20 (1H, m), 7.48 (2H, t, J=7.7 Hz), 7.59-7.65 (1H, m), 8.11-8.15 (2H, m).

FAB-MS; m/z: 853 (M+H)⁺.

### Example 79

### (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-11-en-13-yl

### (2R,3S)-2-hydroxy-3-(isopropoxycarbonylamino)-4,4,4-trifluorobutyrate

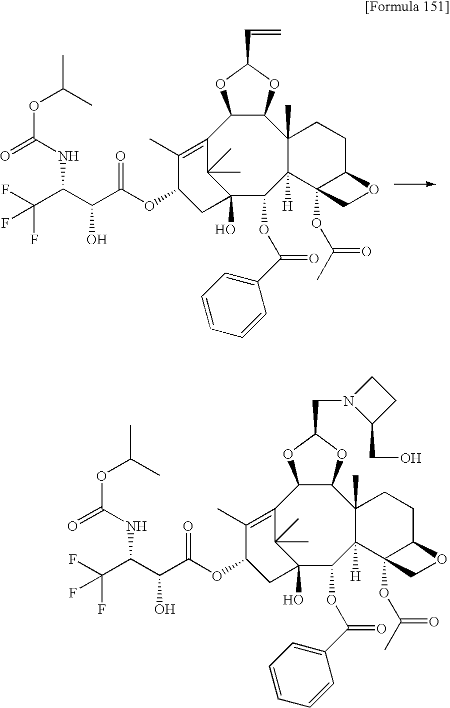

The compound obtained in Example 78, Step 2 and the compound obtained in Reference Example 25 were used as starting materials to perform the same procedure as that of Example 1 and thereby to obtain the title compound as white amorphous solid.

¹H-NMR (400 MHz, CDCl₃) δ: 1.18-2.37 (11H, m), 1.20 (3H, d, J=6.4 Hz), 1.22 (3H, s), 1.25 (3H, d, J=6.4 Hz), 1.48 (3H, s), 1.58 (3H, s), 1.73 (3H, s), 2.34 (3H, s), 2.83 (1H, dd, J=12.7, 4.4 Hz), 2.87-2.94 (2H, m), 3.06-3.15 (1H, m), 3.42 (1H, dd, J=11.5, 2.5 Hz), 3.46-3.54 (2H, m), 3.66 (1H, dd, J=11.3, 2.7 Hz), 4.11 (1H, d, J=7.1 Hz), 4.25 (1H, d, J=8.6 Hz), 4.34 (1H, d, J=8.3 Hz), 4.73 (1H, s), 4.86-5.01 (4H, m), 5.22 (1H, d, J=7.1 Hz), 5.48 (1H, d, J=10.3 Hz), 6.00 (1H, d, J=5.1 Hz), 6.13-6.20 (1H, m), 7.48 (2H, t, J=7.7 Hz), 7.59-7.64 (1H, m), 8.11-8.15 (2H, m).

FAB-MS; m/z: 883 (M+H)⁺.

### Example 80

### (1S,2S,3R,4S,5R,8R,9S,10R,13S)-4-(Acetoxy)-2-(benzoyloxy)-5,20-epoxy-1-hydroxy-9,10-{(1S)-2-[(2S)-2-(11-hydroxy-1-methylethyl)azetidino]ethylidenedioxy}tax-11-en-13-yl

### (2R,3S)-3-(tert-butoxycarbonylamino)-3-(3-fluoro-2-pyridyl)-2-hydroxypropionate

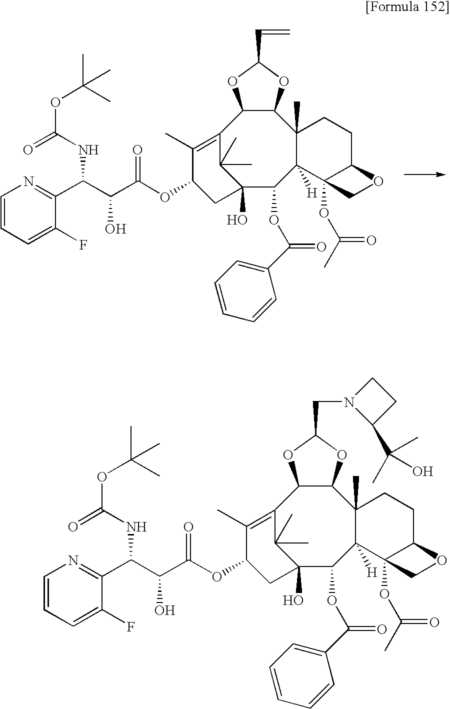

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-3-(3-fluoro-2-pyridyl)-2-hydroxypropionate and the compound obtained in Reference Example 37 were used as starting materials to perform the same procedure as that of Example 1 and thereby to obtain the title compound as white amorphous solid.

¹H-NMR (400 MHz, CDCl₃) δ: 1.03 (3H, s), 1.15 (3H, s), 1.24-2.48 (11H, m), 1.29 (3H, s), 1.40 (9H, s), 1.50 (3H, s), 1.60 (3H, s), 1.79 (3H, s), 2.35 (3H, s), 2.83 (1H, dd, J=12.6, 5.5 Hz), 2.94 (1H, d, J=5.1 Hz), 3.00-3.07 (2H, m), 3.22 (1H, t, J=7.7 Hz), 3.51 (1H, t, J=6.8 Hz), 4.17 (1H, d, J=7.1 Hz), 4.23 (1H, d, J=8.5 Hz), 4.33 (1H, d, J=8.5 Hz), 4.67 (1H, s), 4.88-4.91 (2H, m), 5.24 (1H, d, J=6.8 Hz), 5.65 (1H, d, J=8.3 Hz), 5.98 (1H, d, J=5.1 Hz), 6.09 (1H, t, J=8.8 Hz), 6.20 (1H, d, J=8.5 Hz), 7.28-7.32 (1H, m), 7.43-7.49 (3H, m), 7.58-7.62 (1H, m), 8.13 (2H, d, J=8.3 Hz), 8.40-8.41 (1H, m).

FAB-MS; m/z: 952 (M+H)⁺.

### Example 81

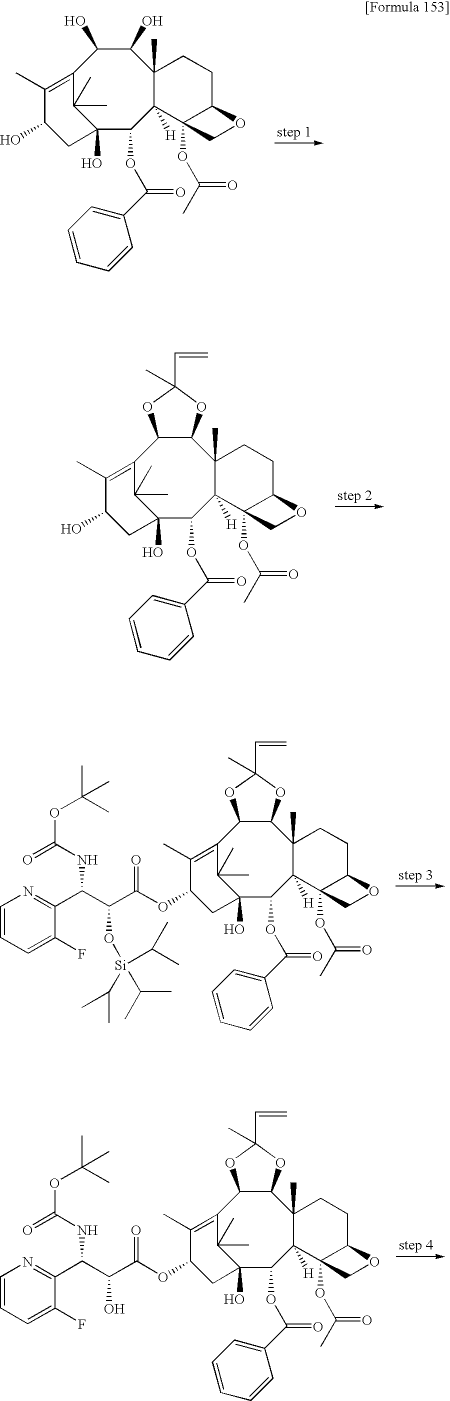

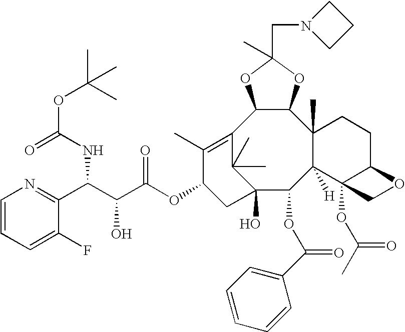

### Step 1: (1S,2S,3R,4S,5R,8R,9S,10R,13S)-4-(Acetoxy)-2-(benzoyloxy)-1,13-dihydroxy-5,20-epoxy-9,10-[(1-methyl)-2-propenylidenedioxy]tax-11-ene

(1S,2S,3R,4S,5R,8R,9S,10R,13S)-4-(Acetoxy)-2-(benzoyloxy)-5,20-epoxy-1,9,10, 13-tetrahydroxytax-11-ene (Bioorg. Med. Chem. Lett., 2002, 12, 2815, 350 mg) and 3,3-dimethoxy-1-butene (J. Am. Chem. Soc., 1935, 57, 2255, 383 mg) were dissolved in tetrahydrofuran (7 ml), and the solution was added with (±)-camphor-10-sulfonic acid (77 mg), and stirred at room temperature for 1 hour. The reaction mixture was added with saturated aqueous sodium hydrogencarbonate, and extracted with ethyl acetate. The organic layer was successively washed with water and saturated brine, and then dried over anhydrous sodium sulfate. The solvent was evaporated under reduced pressure, and then the resulting residue was purified by using thin layer silica gel chromatography [developing solvent; ethyl acetate:n-hexane=1:1 (v/v)] to obtain the title compound (115 mg) as white amorphous solid.

¹H-NMR (400 MHz, CDCl₃) δ: 1.18 (3H, s), 1.40 (3H, s), 1.50 (3H, s), 1.54-2.17 (7H, m), 1.62 (3H, s), 2.00 (3H, s), 2.21-2.39 (1H, m), 2.34 (3H, s), 2.47 (1H, d, J=10.6 Hz), 3.06 (1H, d, J=4.5 Hz), 4.12 (1H, d, J=7.4 Hz), 4.23 (1H, d, J=8.3 Hz), 4.32 (1H, d, J=8.3 Hz), 4.80 (1H, br), 4.85 (1H, s), 5.20 (1H, dd, J=11.0, 1.2 Hz), 5.44 (1H, dd, J=17.5, 1.2 Hz), 5.66 (1H, d, J=7.1 Hz), 5.96 (1H, d, J=5.1 Hz), 6.05 (1H, dd, J=17.5, 11.0 Hz), 7.47 (2H, t, J=7.8 Hz), 7.58 (1H, t, J=7.8 Hz), 8.13 (2H, d, J=7.8 Hz).

FAB-MS; m/z: 583 (M+H)⁺.

### Step 2: (1S,2S,3R,4S,5R,8R,9S,10R,13S)-4-(Acetoxy)-2-(benzoyloxy)-5,20-epoxy-1-hydroxy-9,10-[(1-methyl)-2-propenylidenedioxy]tax-11-en-13-yl

### (2R,3S)-3-(tert-butoxycarbonylamino)-3-(3-fluoro-2-pyridyl)-2-[(triisopropylsilyl)oxy]propionate

The compound obtained in Step 1 mentioned above and (3R,4S)-1-(tert-butoxycarbonyl)-4-(3-fluoro-2-pyridyl)-3-[(triisopropylsilyl)oxy]-2-azetidinone (Bioorg. Med. Chem. Lett., 2004, 14, 3209) were used as starting materials to perform the same procedure as that of Example 31, Step 1 and thereby to obtain the title compound as white amorphous solid.

¹H-NMR (400 MHz, CDCl₃) δ: 0.87-0.93 (21H, m), 1.24-2.04 (6H, m), 1.38 (3H, s), 1.39 (9H, s), 1.46 (3H, s), 1.48 (3H, s), 1.67 (3H, s), 1.84 (3H, s), 2.30-2.41 (2H, m), 2.49 (3H, s), 2.96 (1H, d, J=5.1 Hz), 4.21 (1H, d, J=8.3 Hz), 4.27 (1H, d, J=7.3 Hz), 4.35 (1H, d, J=8.3 Hz), 4.95-4.97 (2H, m), 5.20 (1H, dd, J=11.0, 1.0 Hz), 5.45 (1H, dd, J=17.6, 1.0 Hz), 5.59-5.62 (2H, m), 5.96-6.11 (3H, m), 7.26-7.28 (1H, m), 7.37-7.49 (3H, m), 7.56-7.60 (1H, m), 8.15-8.17 (2H, m), 8.40 (1H, d, J=4.4 Hz).

FAB-MS; m/z: 1021 (M+H)⁺, 1043 (M+Na)⁺.

### Step 3: (1S,2S,3R,4S,5R,8R,9S,10R,13S)-4-(Acetoxy)-2-(benzoyloxy)-5,20-epoxy-1-hydroxy-9,10-[(1-methyl)-2-propenylidenedioxy]tax-11-en-13-yl

### (2R,3S)-3-(tert-butoxycarbonylamino)-3-(3-fluoro-2-pyridyl)-2-hydroxypropionate

The compound obtained in Step 2 mentioned above was used as a starting material to perform the same procedure as that of Example 31, Step 2 and thereby to obtain the title compound as white amorphous solid.

¹H-NMR (400 MHz, CDCl₃) δ: 1.24-2.16 (7H, m), 1.31 (3H, s), 1.41 (9H, s), 1.42 (3H, s), 1.50 (3H, s), 1.66 (3H, s), 1.80 (3H, s), 2.35 (3H, s), 2.45 (1H, dd, J=14.6, 9.5 Hz), 2.93 (1H, d, J=5.1 Hz), 4.21-4.24 (2H, m), 4.32 (1H, d, J=8.3 Hz), 4.68 (1H, dd, J=4.9, 2.7 Hz), 4.92 (1H, s), 5.21 (1H, dd, J=11.0, 1.2 Hz), 5.45 (1H, dd, J=17.6, 1.2 Hz), 5.62-5.66 (2H, m), 5.98 (1H, d, J=5.1 Hz), 6.04-6.20 (3H, m), 7.28-7.32 (1H, m), 7.42-7.49 (3H, m), 7.57-7.61 (1H, m), 8.12-8.14 (2H, m), 8.40-8.41 (1H, m).

FAB-MS; m/z: 865 (M+H)⁺, 887 (M+Na)⁺.

### Step 4: (1S,2S,3R,4S,5R,8R,9S,10R,13S)-4-(Acetoxy)-9,10-[(1-methyl)-2-(azetidino)ethylidenedioxy]-2-(benzoyloxy)-5,20-epoxy-1-hydroxytax-11-en-13-yl

### (2R,3S)-3-(tert-butoxycarbonylamino)-3-(3-fluoro-2-pyridyl)-2-hydroxypropionate

The compound obtained in Step 3 mentioned above was used as a starting material to perform the same procedure as that of Example 1 and thereby to obtain the title compound as white amorphous solid.

¹H-NMR (400 MHz, CDCl₃) δ: 1.24-2.14 (9H, m), 1.29 (3H, s), 1.40 (9H, s), 1.44 (3H, s), 1.55 (3H, s), 1.62 (3H, s), 1.78 (3H, s), 2.35 (3H, s), 2.44 (1H, dd, J=14.6, 9.5 Hz), 2.74 (2H, s), 2.95 (1H, d, J=4.9 Hz), 3.35 (4H, t, J=6.7 Hz), 4.19 (1H, d, J=7.6 Hz), 4.25 (1H, d, J=8.3 Hz), 4.33 (1H, d, J=8.3 Hz), 4.67 (1H, d, J=2.7 Hz), 4.92 (1H, s), 5.58 (1H, d, J=7.6 Hz), 5.64 (1H, d, J=8.8 Hz), 5.98 (1H, d, J=5.1 Hz), 6.09 (1H, t, J=8.7 Hz), 6.20 (1H, d, J=8.5 Hz), 7.28-7.32 (1H, m), 7.42-7.49 (3H, m), 7.58-7.62 (1H, m), 8.13-8.15 (2H, m), 8.40 (1H, dt, J=4.6, 1.2 Hz).

FAB-MS; m/z: 908 (M+H)⁺.

### Example 82

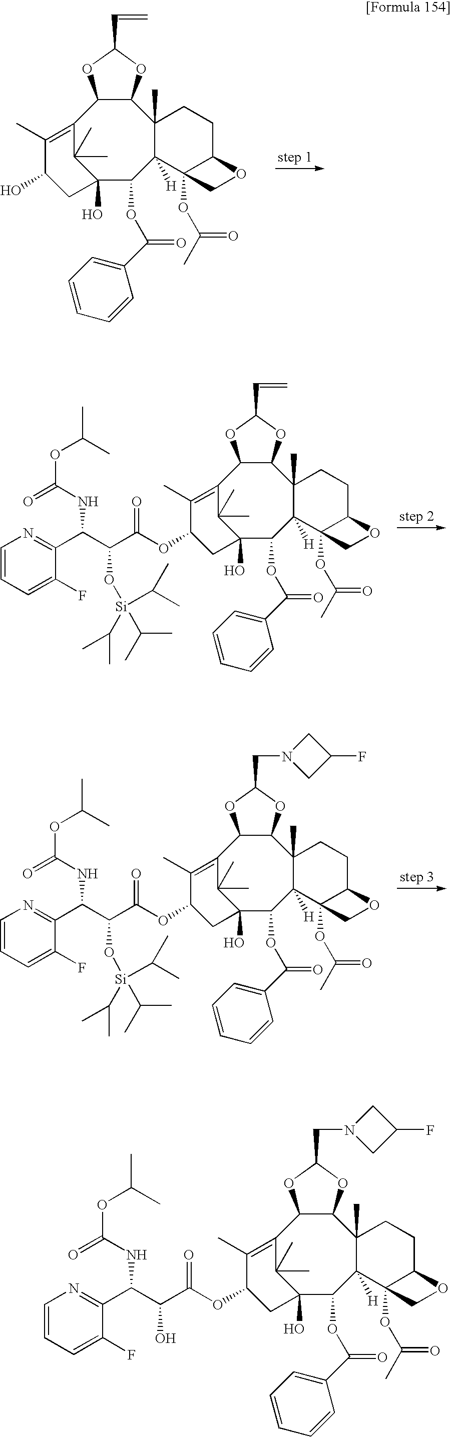

### Step 1: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3S)-3-(3-fluoro-2-pyridyl)-3-(isopropoxycarbonylamino)-2-[(triisopropylsilyl)oxy]propionate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-11-ene and the compound obtained in Reference Example 38 were used as starting materials to perform the same procedure as that of Example 31, Step 1 and thereby to obtain the title compound as white amorphous solid.

¹H-NMR (400 MHz, CDCl₃) δ: 0.87-0.96 (21H, m), 1.16-1.23 (6H, m), 1.37 (3H, s), 1.53 (3H, s), 1.66 (3H, s), 1.80 (3H, s), 1.89-2.39 (7H, m), 2.48 (3H, s), 2.98 (1H, d, J=5.1 Hz), 4.20-4.25 (2H, m), 4.36 (1H, d, J=8.8 Hz), 4.77-4.85 (1H, m), 4.96 (1H, brs), 4.98 (1H, d, J=2.7 Hz), 5.20 (1H, d, J=6.1 Hz), 5.27 (1H, d, J=7.6 Hz), 5.46 (1H, d, J=10.5 Hz), 5.58 (1H, d, J=17.6 Hz), 5.63 (1H, d, J=9.8 Hz), 5.95-6.06 (2H, m), 6.06-6.16 (2H, m), 7.23-7.29 (1H, m), 7.36-7.51 (3H, m), 7.59 (1H, t, J=7.8 Hz), 8.16 (2H, d, J=7.1 Hz), 8.38 (1H, d, J=4.9 Hz).

FAB-MS; m/z: 993 (M+H)⁺.

### Step 2: (1S,2S,3R,4S,5R,8R,9S,10R,13S)-4-(Acetoxy)-2-(benzoyloxy)-5,20-epoxy-9,10-[(1S)-2-(3-fluoroazetidino)ethylidenedioxy]-1-hydroxytax-11-en-13-yl

### (2R,3S)-3-(3-fluoro-2-pyridyl)-3-(isopropoxycarbonylamino)-2-[(triisopropylsilyl)oxy]propionate

The compound obtained in Step 1 mentioned above and the compound obtained in Reference Example 24, Step 2 were used as starting materials to perform the same procedure as that of Example 1 and thereby to obtain the title compound as white amorphous solid.

¹H-NMR (400 MHz, CDCl₃) δ: 0.87-0.98 (21H, m), 1.14-1.23 (6H, m), 1.36 (3H, s), 1.52 (3H, s), 1.62 (3H, s), 1.71-2.39 (7H, m), 1.78 (3H, s), 2.47 (3H, s), 2.82 (1H, dd, J=11.2, 5.6 Hz), 2.90 (1H, dd, J=12.7, 4.6 Hz), 2.96 (1H, d, J=5.9 Hz), 3.25-3.40 (2H, m), 3.79 (2H, brs), 4.17 (1H, d, J=7.6 Hz), 4.21 (1H, d, J=8.8 Hz), 4.36 (1H, d, J=8.3 Hz), 4.76-4.87 (2H, m), 4.95 (1H, brs), 4.97 (1H, brs), 5.04-5.24 (2H, m), 5.62 (1H, d, J=8.8 Hz), 5.97 (1H, d, J=4.9 Hz), 6.04-6.15 (2H, m), 7.24-7.27 (1H, m), 7.40 (1H, t, J=8.8 Hz), 7.47 (2H, t, J=7.8 Hz), 7.59 (1H, t, J=7.3 Hz), 8.16 (2H, d, J=7.8 Hz), 8.38 (1H, d, J=3.9 Hz).

FAB-MS; m/z: 1054 (M+H)⁺.

### Step 3: (1S,2S,3R,4S,5R,8R,9S,10R,13S)-4-(Acetoxy)-2-(benzoyloxy)-5,20-epoxy-9,10-[(1S)-2-(3-fluoroazetidino)ethylidenedioxy]-1-hydroxytax-11-en-13-yl

### (2R,3S)-3-(3-fluoro-2-pyridyl)-2-hydroxy-3-(isopropoxycarbonylamino)propionate

The compound obtained in Step 2 mentioned above was used as a starting material to perform the same procedure as that of Example 31, Step 2 and thereby to obtain the title compound as white amorphous solid.

¹H-NMR (400 MHz, CDCl₃) δ: 1.14-1.23 (6H, m), 1.28 (3H, s), 1.49 (3H, s), 1.61 (3H, s), 1.76 (3H, s), 1.82-2.47 (8H, m), 2.33 (3H, s), 2.82 (1H, dd, J=11.2, 5.6 Hz), 2.87-2.95 (2H, m), 3.25-3.40 (2H, m), 3.72-3.85 (2H, m), 4.13 (1H, d, J=7.6 Hz), 4.23 (1H, d, J=8.8 Hz), 4.32 (1H, d, J=8.3 Hz), 4.70 (1H, d, J=3.2 Hz), 4.80-4.93 (3H, m), 5.04-5.24 (2H, m), 5.70 (1H, d, J=8.8 Hz), 5.99 (1H, d, J=4.9 Hz), 6.11 (1H, t, J=7.3 Hz), 6.26 (1H, d, J=8.3 Hz), 7.27-7.33 (1H, m), 7.40-7.50 (3H, m), 7.60 (1H, t, J=7.3 Hz), 8.13 (2H, d, J=7.8 Hz), 8.39 (1H, d, J=3.9 Hz).

FAB-MS; m/z: 898 (M+H)⁺.

### Example 83

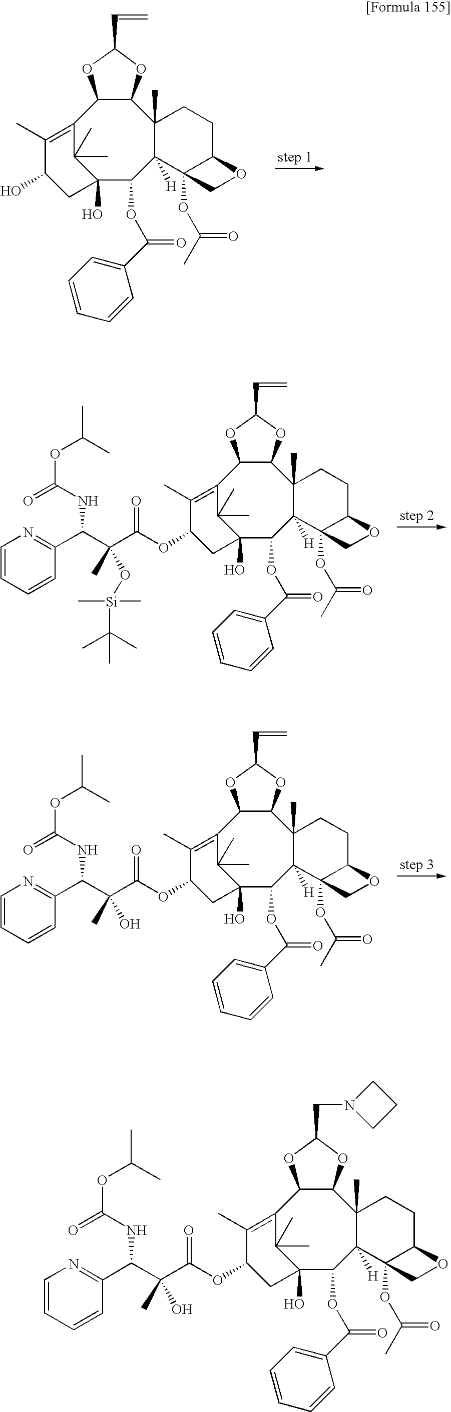

### Step 1: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3S)-2-[(tert-butyldimethylsilyl)oxy]-3-(isopropoxycarbonylamino)-2-methyl-3-(2-pyridyl)propionate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-11-ene and the compound obtained in Reference Example 39 were used as starting materials to perform the same procedure as that of Example 31, Step 1 and thereby to obtain the title compound as white amorphous solid, which was used as it was for the following reaction.

### Step 2: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3S)-2-hydroxy-3-(isopropoxycarbonylamino)-2-methyl-3-(2-pyridyl)propionate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.14-2.23 (7H, m), 1.21 (6H, dd, J=7.8, 6.3 Hz), 1.30 (3H, s), 1.50 (3H, s), 1.53 (3H, s), 1.58 (3H, s), 1.64 (3H, s), 2.50 (3H, s), 2.95 (1H, d, J=4.9 Hz), 4.18 (1H, d, J=7.1 Hz), 4.21 (1H, d, J=8.3 Hz), 4.35 (1H, d, J=8.3 Hz), 4.87-4.95 (2H, m), 5.16 (1H, d, J=10.0 Hz), 5.20-5.23 (2H, m), 5.45 (1H, d, J=10.5 Hz), 5.57 (1H, d, J=17.1 Hz), 5.94-6.04 (3H, m), 6.14-6.20 (2H, m), 7.22-7.27 (1H, m), 7.43-7.50 (3H, m), 7.60 (1H, t, J=7.3 Hz), 7.72 (1H, td, J=7.7, 1.6 Hz), 8.14-8.16 (2H, m), 8.51 (1H, dd, J=4.2, 0.7 Hz).

FAB-MS; m/z: 833 (M+H)⁺.

### Step 3: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3S)-2-hydroxy-3-(isopropoxycarbonylamino)-2-methyl-3-(2-pyridyl)propionate

The compound obtained in Step 2 mentioned above was used as a starting material to perform the same procedure as that of Example 1 and thereby to obtain the title compound as white amorphous solid.

¹H-NMR (400 MHz, CDCl₃) δ: 1.14-2.22 (9H, m), 1.21 (6H, t, J=6.6 Hz), 1.28 (3H, s), 1.49 (3H, s), 1.53 (3H, s), 1.55 (3H, s), 1.60 (3H, s), 2.50 (3H, s), 2.73 (1H, dd, J=12.5, 5.6 Hz), 2.81 (1H, dd, J=12.5, 4.4 Hz), 2.93 (1H, d, J=5.1 Hz), 3.30-3.37 (4H, m), 4.11 (1H, d, J=7.3 Hz), 4.21 (1H, d, J=8.3 Hz), 4.34 (1H, d, J=8.3 Hz), 4.82-4.94 (3H, m), 5.12-5.16 (2H, m), 5.96 (2H, d, J=5.1 Hz), 6.13-6.19 (2H, m), 7.22-7.25 (1H, m), 7.42-7.50 (3H, m), 7.58-7.62 (1H, m), 7.72 (1H, td, J=7.8, 1.7 Hz), 8.14-8.16 (2H, m), 8.49-8.51 (1H, m).

FAB-MS; m/z: 876 (M+H)⁺.

### Example 84

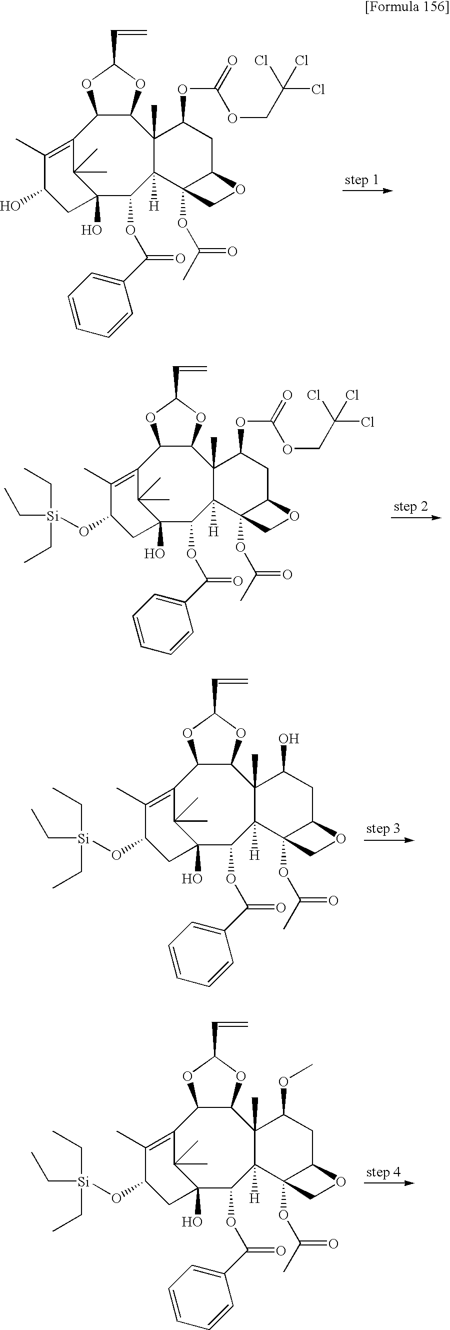

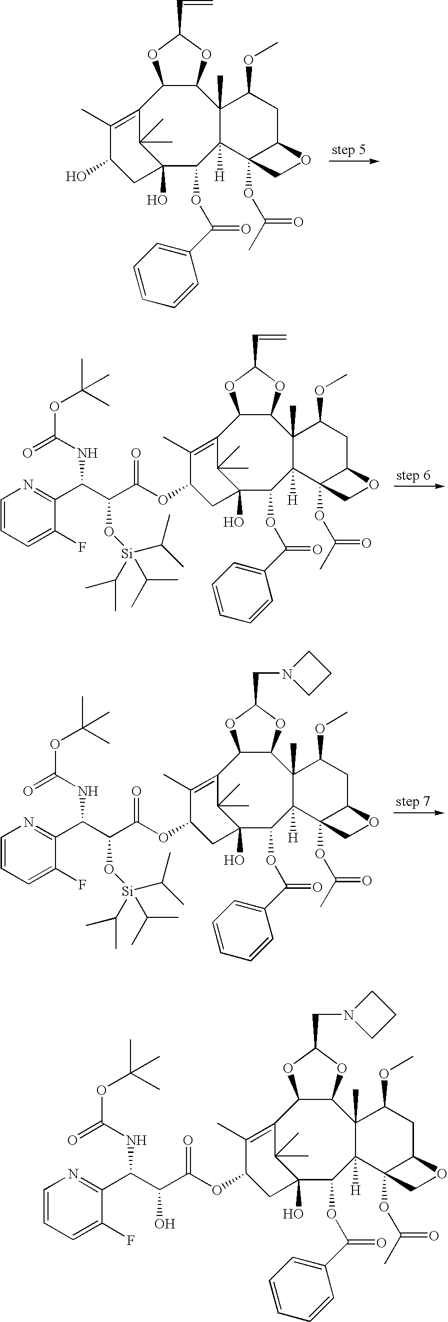

### Step 1: (1S,2S,3R,4S,5R,7S,8S,9S,10R,13S)-4-(Acetoxy)-2-(benzoyloxy)-5,20-epoxy-1-hydroxy-9,10-[(1S)-2-propenylidenedioxy]-7-[(2,2,2-trichloroethoxycarbonyl)oxy]-13-[(triethylsilyl)oxy]tax-11-ene

(1S,2S,3R,4S,5R,7S,8S,9S,10R,13S)-4-(Acetoxy)-2-(benzoyloxy)-1,13-dihydroxy-5,20-epoxy-9,10-[(1S)-2-propenylidenedioxy]-7-[(2,2,2-trichloroethoxycarbonyl)oxy]tax-11-ene (WO96/33998, 2.60 g) and 2,6-lutidine (0.68 ml) were dissolved in dichloromethane (52 ml), and cooled to −45° C. Then, the solution was added dropwise with triethylsilyl trifluoromethanesulfonate (1.16 ml), and stirred at the same temperature for 1 hour. The reaction mixture was added with saturated aqueous sodium hydrogencarbonate, and the organic layer was washed with saturated brine, and then dried over anhydrous sodium sulfate. The solvent was evaporated under reduced pressure, and then the resulting residue was added with n-hexane/ether. The deposited solid was collected by filtration, and dried to obtain the title compound (2.60 g) as colorless solid.

¹H-NMR (400 MHz, CDCl₃) δ: 0.56-0.75 (6H, m), 1.01 (9H, t, J=7.8 Hz), 1.22 (3H, s), 1.58 (3H, s), 1.61 (3H, s), 1.76 (3H, s), 1.89 (3H, s), 2.05-2.40 (3H, m), 2.28 (3H, s), 3.17 (1H, d, J=5.3 Hz), 4.02 (1H, d, J=8.3 Hz), 4.31 (1H, d, J=8.3 Hz), 4.38 (1H, d, J=8.3 Hz), 4.66 (1H, d, J=11.7 Hz), 4.86 (1H, d, J=11.7 Hz), 4.91 (1H, t, J=4.9 Hz), 4.97 (1H, t, J=8.8 Hz), 5.05-5.15 (2H, m), 5.30 (1H, d, J=8.3 Hz), 5.45 (1H, d, J=10.7 Hz), 5.56 (1H, d, J=17.6 Hz), 5.90 (1H, d, J=5.3 Hz), 6.03 (1H, ddd, J=17.1, 10.7, 5.9 Hz), 7.47 (2H, t, J=7.8 Hz), 7.58 (1H, t, J=7.8 Hz), 8.12 (2H, d, J=7.8 Hz).

### Step 2: (1S,2S,3R,4S,5R,7S,8S,9S,10R,13S)-4-(Acetoxy)-2-(benzoyloxy)-1,7-dihydroxy-5,20-epoxy-9,10-[(1S)-2-propenylidenedioxy]-13-[(triethylsilyl)oxy]tax-1-ene

The compound obtained in Step 1 mentioned above (2.60 g) was dissolved in ethyl acetate, and the solution was added with acetic acid (8.48 ml) and active zinc (12.8 g), and stirred at room temperature for 2 hours. The insoluble matters were removed by filtration through Celite, and the filtrate was successively washed with saturated aqueous sodium hydrogencarbonate and saturated brine, and then dried over anhydrous sodium sulfate. The solvent was evaporated under reduced pressure, and then the resulting residue was purified by silica gel chromatography [developing solvent; chloroform:acetone=15:1 (v/v)] to obtain the title compound (1.05 g) as colorless solid.

¹H-NMR (400 MHz, CDCl₃) δ: 0.59-0.73 (6H, m), 1.01 (9H, t, J=7.8 Hz), 1.25 (3H, s), 1.61 (3H, s), 1.65 (3H, s), 1.85 (3H, s), 1.86 (1H, s), 2.03-2.34 (4H, m), 2.27 (3H, s), 2.95 (1H, d, J=4.9 Hz), 3.97 (1H, d, J=7.4 Hz), 4.07 (1H, br), 4.25 (1H, d, J=8.3 Hz), 4.39 (1H, d, J=8.3 Hz), 4.55 (1H, brd, J=6.8 Hz), 4.98 (1H, t, J=8.3 Hz), 5.10 (1H, s), 5.19 (1H, d, J=6.3 Hz), 5.25 (1H, d, J=6.8 Hz), 5.43 (1H, d, J=10.8 Hz), 5.56 (1H, d, J=17.1 Hz), 5.91-6.10 (2H, m), 7.47 (2H, t, J=7.8 Hz), 7.59 (1H, t, J=7.8 Hz), 8.12 (2H, d, J=7.8 Hz).

### Step 3: (1S,2S,3R,4S,5R,7S,8S,9S,10R,13S)-4-(Acetoxy)-2-(benzoyloxy)-5,20-epoxy-1-hydroxy-7-methoxy-9,10-[(1S)-2-propenylidenedioxy]-13-[(triethylsilyl)oxy]tax-11-ene

The compound obtained in Step 2 mentioned above (1.00 g) was dissolved in 2,6-di-tert-butylpyridine (3.5 ml), and the solution was added with methyl trifluoromethanesulfonate (0.80 ml), and stirred at room temperature for 4 hours. The reaction mixture was diluted with ethyl acetate, and the organic layer was washed with saturated brine, and then dried over anhydrous sodium sulfate. The solvent was evaporated under reduced pressure, and then the resulting residue was purified by silica gel chromatography [developing solvent; ethyl acetate:n-hexane=1:5 to 1:2 (v/v)] to obtain the title compound (180 mg) as colorless solid.

¹H-NMR (400 MHz, CDCl₃) δ: 0.55-0.75 (6H, m), 1.01 (9H, t, J=7.8 Hz), 1.16 (3H, s), 1.56 (3H, s), 1.71 (3H, s), 1.89 (3H, s), 1.95-2.05 (2H, m), 2.10 (1H, dd, J=15.2, 8.8 Hz), 2.24 (1H, dd, J=15.2, 8.8 Hz), 2.27 (3H, s), 2.48-2.55 (1H, m), 3.25 (1H, d, J=5.9 Hz), 3.31 (1H, dd, J=10.5, 4.6 Hz), 3.39 (3H, s), 4.31 (1H, d, J=8.3 Hz), 4.40 (1H, d, J=8.3 Hz), 4.42 (1H, d, J=9.1 Hz), 4.86 (1H, dd, J=8.5, 5.6 Hz), 4.95 (1H, dd, J=8.8, 8.3 Hz), 5.14 (1H, d, J=6.1 Hz), 5.34 (1H, d, J=9.1 Hz), 5.46 (1H, d, J=10.7 Hz), 5.58 (1H, d, J=17.6 Hz), 5.82 (1H, d, J=5.9 Hz), 6.12 (1H, ddd, J=17.6, 10.7, 6.1 Hz), 7.47 (2H, t, J=7.3 Hz), 7.58 (1H, t, J=7.3 Hz), 8.08 (2H, d, J=7.3 Hz).

### Step 4: (1S,2S,3R,4S,5R,7S,8S,9S,10R,13S)-4-(Acetoxy)-2-(benzoyloxy)-1,13-dihydroxy-5,20-epoxy-7-methoxy-9,10-[(1S)-2-propenylidenedioxy]tax-11-ene

The compound obtained in Step 3 mentioned above (180 mg) was dissolved in pyridine (4 ml), and the solution was added with hydrogen fluoride/pyridine (1 ml) under ice cooling, returned to room temperature, and stirred overnight. The reaction mixture was cooled in ice, diluted with ethyl acetate, and neutralized with saturated aqueous sodium hydrogencarbonate. The organic layer was successively washed with 1 N aqueous hydrochloric acid and saturated brine, and then dried over anhydrous sodium sulfate. The solvent was evaporated under reduced pressure, and then the resulting residue was purified by thin layer silica gel column chromatography [developing solvent; ethyl acetate:n-hexane=1:1 (v/v)] to obtain the title compound (110 mg) as colorless solid.

¹H-NMR (400 MHz, CDCl₃) δ: 1.12 (3H, s), 1.56 (3H, s), 1.61 (3H, s), 1.77 (1H, s), 1.96 (3H, s), 1.85-2.40 (5H, m), 2.32 (3H, s), 3.22 (1H, d, J=5.1 Hz), 3.37 (3H, s), 3.41 (1H, dd, J=7.8, 4.9 Hz), 4.29 (1H, dd, J=7.8 Hz), 4.36 (1H, d, J=8.1 Hz), 4.43 (1H, d, J=7.8 Hz), 4.82-4.90 (2H, m), 5.17 (1H, d, J=5.9 Hz), 5.36 (1H, d, J=8.1 Hz), 5.48 (1H, d, J=10.3 Hz), 5.59 (1H, d, J=17.1 Hz), 5.92 (1H, d, J=5.1 Hz), 6.14 (1H, ddd, J=17.1, 10.3, 5.9 Hz), 7.47 (2H, t, J=7.3 Hz), 7.59 (1H, t, J=7.3 Hz), 8.11 (2H, d, J=7.6 Hz).

### Step 5: (1S,2S,3R,4S,5R,7S,8S,9S,10R,13S)-4-(Acetoxy)-2-(benzoyloxy)-5,20-epoxy-1-hydroxy-7-methoxy-9,10-[(1S)-2-propenylidenedioxy]tax-11-en-13-yl

### (2R,3S)-3-(tert-butoxycarbonylamino)-3-(3-fluoro-2-pyridyl)-2-[(triisopropylsilyl)oxy]propionate

The compound obtained in Step 4 mentioned above and (3R,4S)-1-(tert-butoxycarbonyl)-4-(3-fluoro-2-pyridyl)-3-[(triisopropylsilyl)oxy]-2-azetidinone were used as starting materials to perform the same procedure as that of Example 31, Step 1 and thereby to obtain the title compound as white amorphous solid.

¹H-NMR (400 MHz, CDCl₃) δ: 0.87-0.96 (21H, m), 1.28 (3H, s), 1.38 (9H, s), 1.56 (3H, s), 1.59 (3H, s), 1.80 (3H, s), 1.97-2.46 (5H, m), 2.49 (3H, s), 3.27 (1H, d, J=6.3 Hz), 3.29-3.35 (1H, m), 3.39 (3H, s), 4.32 (1H, d, J=8.8 Hz), 4.36 (1H, d, J=9.0 Hz), 4.48 (1H, d, J=8.3 Hz), 4.83-4.90 (1H, m), 4.93 (1H, brs), 5.14 (1H, d, J=5.9 Hz), 5.32 (1H, d, J=8.8 Hz), 5.46 (1H, d, J=10.5 Hz), 5.55-5.66 (2H, m), 5.92 (1H, d, J=5.4 Hz), 6.02-6.18 (3H, m), 7.25-7.29 (1H, m), 7.40 (1H, t, J=9.8 Hz), 7.47 (2H, t, J=7.6 Hz), 7.57 (1H, t, J=7.6 Hz), 8.12 (2H, d, J=7.8 Hz), 8.40 (1H, d, J=4.4 Hz).

FAB-MS; m/z: 1037 (M+H)⁺.

### Step 6: (1S,2S,3R,4S,5R,7S,8S,9S,10R,13S)-4-(Acetoxy)-9,10-[(1S)-2-(azetidino)ethylidenedioxy]-2-(benzoyloxy)-5,20-epoxy-1-hydroxy-7-methoxytax-11-en-13-yl

### (2R,3S)-3-(tert-butoxycarbonylamino)-3-(3-fluoro-2-pyridyl)-2-[(triisopropylsilyl)oxy]propionate

The compound obtained in Step 5 mentioned above was used as a starting material to perform the same procedure as that of Example 1 and thereby to obtain the title compound as white amorphous solid.

¹H-NMR (400 MHz, CDCl₃) δ: 0.83-0.94 (21H, m), 1.27 (3H, s), 1.38 (9H, s), 1.51 (3H, s), 1.55 (3H, s), 1.59-2.52 (7H, m), 1.78 (3H, s), 2.48 (3H, s), 2.81-2.86 (2H, m), 3.23 (1H, d, J=5.9 Hz), 3.26-3.38 (5H, m), 3.35 (3H, s), 4.18 (1H, d, J=8.8 Hz), 4.30 (1H, d, J=8.1 Hz), 4.49 (1H, d, J=8.3 Hz), 4.74 (1H, t, J=5.1 Hz), 4.85 (1H, t, J=6.6 Hz), 4.93 (1H, brs), 5.21 (1H, d, J=9.0 Hz), 5.62 (1H, d, J=10.0 Hz), 5.89 (1H, d, J=5.4 Hz), 6.05 (1H, d, J=10.7 Hz), 6.11 (1H, t, J=9.0 Hz), 7.24-7.28 (1H, m), 7.40 (1H, t, J=10.5 Hz), 7.47 (2H, t, J=7.8 Hz), 7.57 (1H, t, J=8.5 Hz), 8.12 (2H, d, J=9.0 Hz), 8.40 (1H, d, J=4.2 Hz).

FAB-MS; m/z: 1080 (M+H)⁺.

### Step 7: (1S,2S,3R,4S,5R,7S,8S,9S,10R,13S)-4-(Acetoxy)-9,10-[(1S)-2-(azetidino)ethylidenedioxy]-2-(benzoyloxy)-5,20-epoxy-1-hydroxy-7-methoxytax-11-en-13-yl

### (2R,3S)-3-(tert-butoxycarbonylamino)-3-(3-fluoro-2-pyridyl)-2-hydroxypropionate

The compound obtained in Step 6 mentioned above was used as a starting material to perform the same procedure as that of Example 31, Step 2 and thereby to obtain the title compound as white amorphous solid.

¹H-NMR (400 MHz, CDCl₃) δ: 1.24 (3H, s), 1.39 (9H, s), 1.54 (3H, s), 1.60 (3H, s), 1.75-2.51 (8H, m), 1.78 (3H, s), 2.35 (3H, s), 2.87 (2H, d, J=5.1 Hz), 3.12 (1H, d, J=4.6 Hz), 3.33-3.39 (5H, m), 3.35 (3H, s), 4.27 (2H, d, J=9.0 Hz), 4.42 (1H, d, J=8.5 Hz), 4.65 (1H, d, J=1.5 Hz), 4.76 (1H, t, J=5.1 Hz), 4.86 (1H, dd, J=7.1, 3.9 Hz), 5.26 (1H, d, J=8.5 Hz), 5.65 (1H, d, J=8.3 Hz), 5.92 (1H, d, J=5.1 Hz), 6.10 (1H, t, J=7.8 Hz), 6.19 (1H, d, J=9.0 Hz), 7.28-7.33 (1H, m), 7.42-7.50 (3H, m), 7.59 (1H, t, J=8.5 Hz), 8.10 (2H, d, J=8.5 Hz), 8.40 (1H, d, J=5.6 Hz).

FAB-MS; m/z: 924 (M+H)⁺.

### Example 85

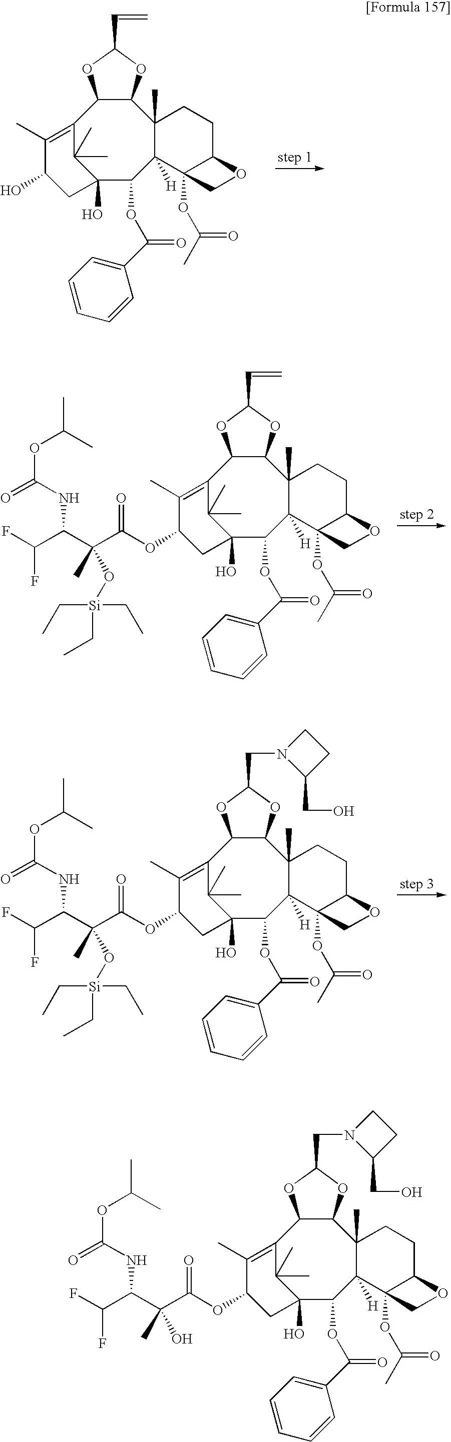

### Step 1: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3R)-4,4-difluoro-3-(isopropoxycarbonylamino)-2-methyl-2-[(triethylsilyl)oxy]butyrate

(1S,2S,3R,4S,5R,8R,9S,10R,13S)-4-(Acetoxy)-2-(benzoyloxy)-1,13-dihydroxy-5, 20-epoxy-9,10-[(1S)-2-propenylidenedioxy]tax-11-ene and the compound obtained in Reference Example 40 were used as starting materials to perform the same procedure as that of Example 31, Step 1 and thereby to obtain the title compound as white amorphous solid, and used as it was for the following reaction.

### Step 2: (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-11-en-13-yl

### (2R,3R)-4,4-difluoro-3-(isopropoxycarbonylamino)-2-methyl-2-[(triethylsilyl)oxy]butyrate

The compound obtained in Step 1 mentioned above and the compound obtained in Reference Example 25 were used as starting materials to perform the same procedure as that of Example 1 and thereby to obtain the title compound as white amorphous solid.

¹H-NMR (400 MHz, CDCl₃) δ: 0.61-0.80 (6H, m), 0.98 (9H, t, J=8.0 Hz), 1.14 (3H, d, J=6.1 Hz), 1.18 (3H, d, J=6.1 Hz), 1.26 (3H, s), 1.33 (3H, s), 1.50 (3H, s), 1.60 (3H, s), 1.73 (3H, s), 1.80-2.32 (10H, m), 2.49 (3H, s), 2.81 (1H, dd, J=12.9, 5.1 Hz), 2.85-2.92 (2H, m), 3.10 (1H, q, J=8.1 Hz), 3.42 (1H, d, J=11.7 Hz), 3.44-3.52 (2H, m), 3.63 (1H, dd, J=11.3, 3.2 Hz), 4.12 (1H, d, J=7.1 Hz), 4.24 (1H, d, J=8.3 Hz), 4.31 (1H, d, J=8.5 Hz), 4.33-4.38 (1H, m), 4.77-4.87 (2H, m), 4.91 (1H, brs), 5.03 (1H, d, J=10.2 Hz), 5.18 (1H, d, J=7.6 Hz), 5.95 (1H, d, J=4.9 Hz), 5.88 (1H, dt, J=54.1, 4.9 Hz), 6.12 (1H, t, J=8.5 Hz), 7.49 (2H, t, J=7.6 Hz), 7.61 (1H, t, J=8.3 Hz), 8.16 (2H, d, J=8.0 Hz).

FAB-MS; m/z: 993 (M+H)⁺.

### Step 3: (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-11-en-13-yl

### (2R,3R)-4,4-difluoro-2-hydroxy-3-(isopropoxycarbonylamino)-2-methylbutyrate

The compound obtained in Step 2 mentioned above was used as a starting material to perform the same procedure as that of Example 31, Step 2 and thereby to obtain the title compound as white amorphous solid.

¹H-NMR (400 MHz, CDCl₃) δ: 1.20 (3H, d, J=7.1 Hz), 1.23 (3H, d, J=6.1 Hz), 1.27 (3H, s), 1.48 (3H, s), 1.60 (3H, s), 1.63 (3H, s), 1.75 (3H, s), 1.79-2.35 (11H, m), 2.42 (3H, s), 2.83 (1H, dd, J=12.9, 5.1 Hz), 2.90 (1H, dd, J=12.4, 5.4 Hz), 2.94 (1H, d, J=5.1 Hz), 3.10 (1H, q, J=8.1 Hz), 3.43 (1H, d, J=11.7 Hz), 3.45-3.53 (2H, m), 3.65 (1H, dd, J=11.3, 2.3 Hz), 4.12 (1H, d, J=7.1 Hz), 4.26 (1H, d, J=8.3 Hz), 4.32 (1H, d, J=8.5 Hz), 4.35-4.50 (1H, m), 4.85-4.95 (3H, m), 5.21 (1H, d, J=7.6 Hz), 5.46 (1H, d, J=8.8 Hz), 5.99 (1H, d, J=4.9 Hz), 6.09 (1H, dt, J=54.1, 2.7 Hz), 6.20 (1H, t, J=8.5 Hz), 7.49 (2H, t, J=7.6 Hz), 7.61 (1H, t, J=8.3 Hz), 8.15 (2H, d, J=8.0 Hz).

FAB-MS; m/z: 879 (M+H)⁺.

### Example 86

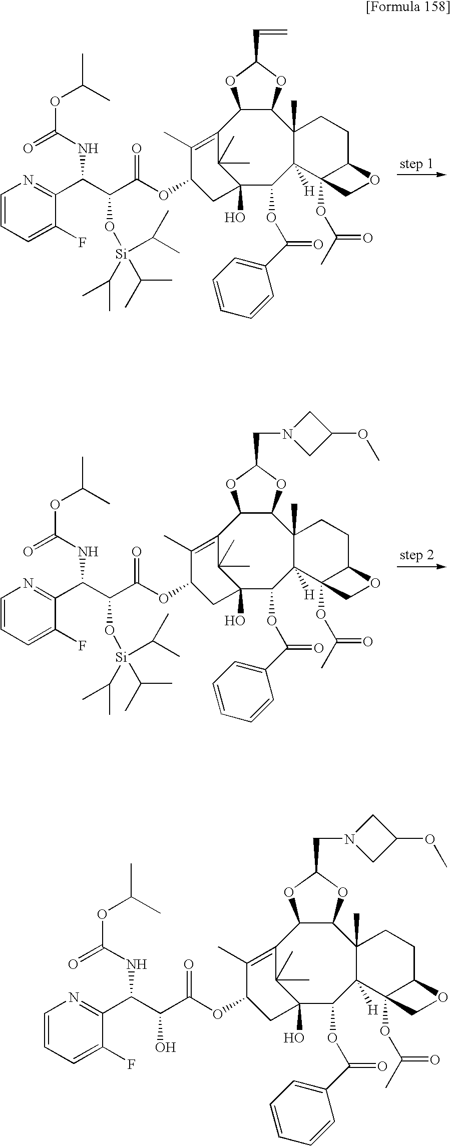

### Step 1: (1S,2S,3R,4S,5R,8R,9S,10R,13S)-4-(Acetoxy)-2-(benzoyloxy)-5,20-epoxy-1-hydroxy-9,10-[(1S)-2-(3-methoxyazetidino)ethylidenedioxy]tax-11-en-13-yl

### (2R,3S)-3-(3-fluoro-2-pyridyl)-3-(isopropoxycarbonylamino)-2-[(triisopropylsilyl)oxy]propionate

The compound obtained in Example 82, Step 1 and the compound obtained in Reference Example 1 were used as starting materials to perform the same procedure as that of Example 1 and thereby to obtain the title compound as white amorphous solid.

¹H-NMR (400 MHz, CDCl₃) δ: 0.87-0.98 (21H, m), 1.16-1.23 (6H, m), 1.35 (3H, s), 1.51 (3H, s), 1.55-2.38 (7H, m), 1.62 (3H, s), 1.78 (3H, s), 2.47 (3H, s), 2.79 (1H, dd, J=12.4, 6.1 Hz), 2.87 (1H, dd, J=12.9, 4.6 Hz), 2.96 (1H, d, J=5.4 Hz), 3.04-3.12 (2H, m), 3.27 (3H, s), 3.70-3.78 (2H, m), 4.04-4.12 (1H, m), 4.16 (1H, d, J=7.8 Hz), 4.21 (1H, d, J=8.5 Hz), 4.36 (1H, d, J=7.8 Hz), 4.77-4.86 (2H, m), 4.95 (1H, brs), 4.97 (1H, d, J=3.4 Hz), 5.18 (1H, d, J=7.6 Hz), 5.62 (1H, d, J=9.5 Hz), 5.97 (1H, d, J=5.1 Hz), 6.05-6.15 (2H, m), 7.24-7.29 (1H, m), 7.40 (1H, t, J=9.1 Hz), 7.47 (2H, t, J=7.8 Hz), 7.59 (1H, t, J=7.4 Hz), 8.16 (2H, d, J=8.5 Hz), 8.38 (1H, d, J=4.6 Hz).

FAB-MS; m/z: 1066 (M+H)⁺.

### Step 2: (1S,2S,3R,4S,5R,8R,9S,10R,13S)-4-(Acetoxy)-2-(benzoyloxy)-5,20-epoxy-1-hydroxy-9,10-[(1S)-2-(3-methoxyazetidino)ethylidenedioxy]tax-11-en-13-yl

### (2R,3S)-3-(3-fluoro-2-pyridyl)-2-hydroxy-3-(isopropoxycarbonylamino)propionate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.15-1.24 (6H, m), 1.28 (3H, s), 1.49 (3H, s), 1.54-2.46 (8H, m), 1.61 (3H, s), 1.76 (3H, s), 2.33 (3H, s), 2.79 (1H, dd, J=12.4, 5.6 Hz), 2.88 (1H, dd, J=12.4, 4.6 Hz), 2.92 (1H, d, J=5.6 Hz), 3.06-3.11 (2H, m), 3.27 (3H, s), 3.69-3.78 (2H, m), 4.03-4.15 (2H, m), 4.23 (1H, d, J=9.0 Hz), 4.32 (1H, d, J=9.0 Hz), 4.70 (1H, d, J=2.7 Hz), 4.81-4.93 (3H, m), 5.21 (1H, d, J=7.1 Hz), 5.70 (1H, d, J=8.3 Hz), 5.99 (1H, d, J=5.1 Hz), 6.11 (1H, t, J=8.7 Hz), 6.25 (1H, d, J=7.8 Hz), 7.27-7.32 (1H, m), 7.41-7.51 (3H, m), 7.60 (1H, t, J=7.2 Hz), 8.13 (2H, d, J=8.5 Hz), 8.39 (1H, d, J=4.6 Hz).

FAB-MS; m/z: 910 (M+H)⁺.

### Example 87

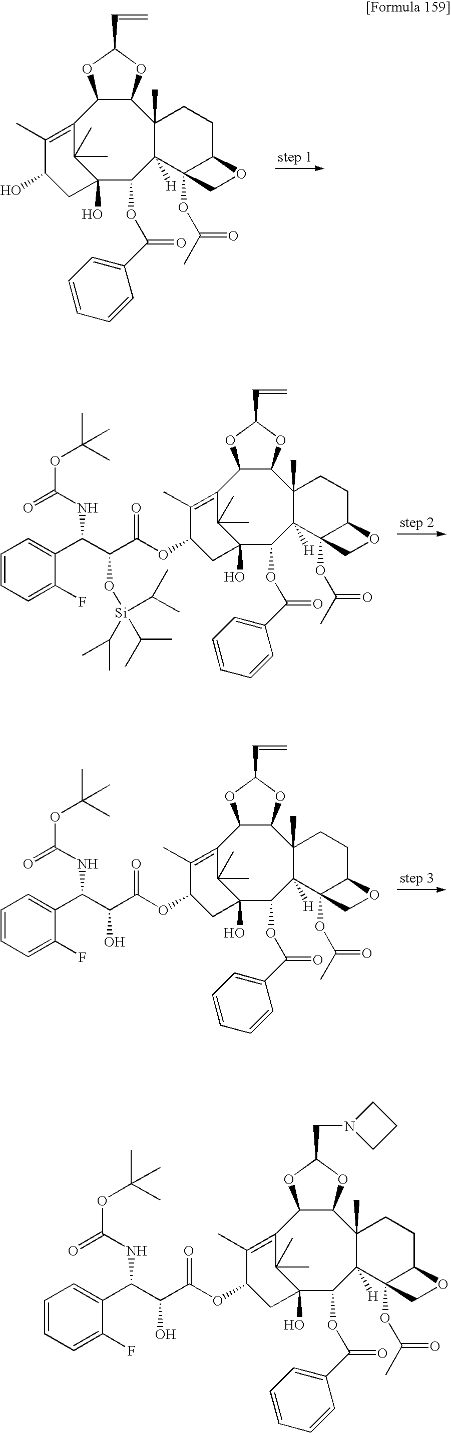

### Step 1: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3S)-3-(tert-butoxycarbonylamino)-3-(2-fluorophenyl)-2-[(triisopropylsilyl)oxy]propionate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-11-ene and the compound obtained in Reference Example 41, Step 4 were used as starting materials to perform the same procedure as that of Example 31, Step 1 and thereby to obtain the title compound as white amorphous solid.

¹H-NMR (400 MHz, CDCl₃) δ: 0.72-1.06 (21H, m), 1.08-2.37 (7H, m), 1.35 (3H, s), 1.38 (9H, s), 1.53 (3H, s), 1.65 (3H, s), 1.81 (3H, s), 2.47 (3H, s), 2.97 (1H, d, J=5.1 Hz), 4.21-4.23 (2H, m), 4.34 (1H, d, J=8.3 Hz), 4.93 (2H, s), 5.20 (1H, d, J=6.1 Hz), 5.27 (1H, d, J=6.8 Hz), 5.45 (2H, d, J=10.2 Hz), 5.54-5.60 (2H, m), 5.96-6.04 (2H, m), 6.15 (1H, t, J=8.3 Hz), 7.04-7.09 (1H, m), 7.14 (1H, t, J=7.6 Hz), 7.25-7.34 (2H, m), 7.47 (2H, t, J=7.3 Hz), 7.56-7.60 (1H, m), 8.14-8.16 (2H, m).

FAB-MS; m/z: 1006 (M+H)⁺, 1028 (M+Na)⁺.

### Step 2: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-1′-en-13-yl

### (2R,3S)-3-(tert-butoxycarbonylamino)-3-(2-fluorophenyl)-2-hydroxypropionate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.24-2.10 (6H, m), 1.28 (3H, s), 1.41 (9H, s), 1.49 (3H, s), 1.64 (3H, s), 1.79 (3H, s), 2.29 (3H, s), 2.38 (1H, dd, J=15.1, 9.5 Hz), 2.94 (1H, d, J=4.9 Hz), 4.18 (1H, d, J=6.8 Hz), 4.25 (1H, d, J=8.3 Hz), 4.32 (1H, d, J=8.3 Hz), 4.46 (1H, brs), 4.62 (1H, brs), 4.92 (1H, s), 5.25 (1H, d, J=6.1 Hz), 5.32 (1H, d, J=6.8 Hz), 5.47 (1H, d, J=10.5 Hz), 5.56-5.67 (3H, m), 5.97-6.06 (2H, m), 6.15 (1H, t, J=8.4 Hz), 7.05-7.10 (1H, m), 7.14-7.18 (1H, m), 7.27-7.33 (1H, m), 7.40-7.52 (3H, m), 7.58-7.62 (1H, m), 8.11-8.13 (2H, m).

FAB-MS; m/z: 850 (M+H)⁺, 872 (M+Na)⁺.

### Step 3: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3S)-3-(tert-butoxycarbonylamino)-3-(2-fluorophenyl)-2-hydroxypropionate

The compound obtained in Step 2 mentioned above was used as a starting material to perform the same procedure as that of Example 1 and thereby to obtain the title compound as white amorphous solid.

¹H-NMR (400 MHz, CDCl₃) δ: 1.24-2.17 (9H, m), 1.26 (3H, s), 1.40 (9H, s), 1.49 (3H, s), 1.61 (3H, s), 1.77 (3H, s), 2.29 (3H, s), 2.34-2.40 (1H, m), 2.75 (1H, dd, J=12.5, 5.9 Hz), 2.83 (1H, dd, J=12.5, 4.2 Hz), 2.92 (1H, d, J=4.9 Hz), 3.32-3.40 (4H, m), 4.09-4.13 (1H, m), 4.24 (1H, d, J=8.5 Hz), 4.31 (1H, d, J=8.5 Hz), 4.62 (1H, s), 4.88 (1H, dd, J=5.9, 4.2 Hz), 4.91 (1H, s), 5.22 (1H, d, J=7.1 Hz), 5.64 (2H, s), 6.00 (1H, d, J=4.9 Hz), 6.13 (1H, t, J=8.5 Hz), 7.05-7.10 (1H, m), 7.16 (1H, td, J=7.5, 1.1 Hz), 7.26-7.32 (1H, m), 7.37-7.49 (3H, m), 7.58-7.62 (1H, m), 8.11-8.13 (2H, m).

FAB-MS; m/z: 893 (M+H)⁺.

### Example 88

### (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-11-en-13-yl

### (2R,3S)-3-(tert-butoxycarbonylamino)-3-(2-fluorophenyl)-2-hydroxypropionate

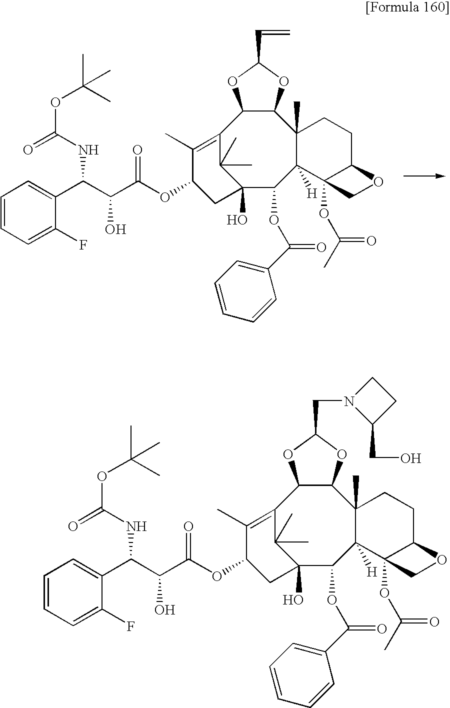

The compound obtained in Example 87, Step 2 and the compound obtained in Reference Example 25 were used as starting materials to perform the same procedure as that of Example 1 and thereby to obtain the title compound as white amorphous solid.

¹H-NMR (400 MHz, CDCl₃) δ: 1.24-2.13 (9H, m), 1.25 (3H, s), 1.41 (9H, s), 1.49 (3H, s), 1.59 (3H, s), 1.77 (3H, s), 2.20-2.40 (2H, m), 2.29 (3H, s), 2.83 (1H, dd, J=12.8, 4.5 Hz), 2.88-2.92 (2H, m), 3.11 (1H, dd, J=16.2, 8.7 Hz), 3.43 (1H, dd, J=11.5, 2.4 Hz), 3.46-3.52 (2H, m), 3.66 (1H, dd, J=11.5, 2.9 Hz), 4.12 (1H, d, J=7.3 Hz), 4.24 (1H, d, J=8.5 Hz), 4.31 (1H, d, J=8.5 Hz), 4.62 (1H, s), 4.88-4.91 (2H, m), 5.24 (1H, d, J=6.8 Hz), 5.60-5.67 (2H, m), 5.99 (1H, d, J=4.9 Hz), 6.13 (1H, t, J=8.1 Hz), 7.05-7.10 (1H, m), 7.14-7.18 (1H, m), 7.26-7.32 (1H, m), 7.37-7.49 (3H, m), 7.58-7.62 (1H, m), 8.11-8.13 (2H, m).

FAB-MS; m/z: 923 (M+H)⁺.

### Example 89

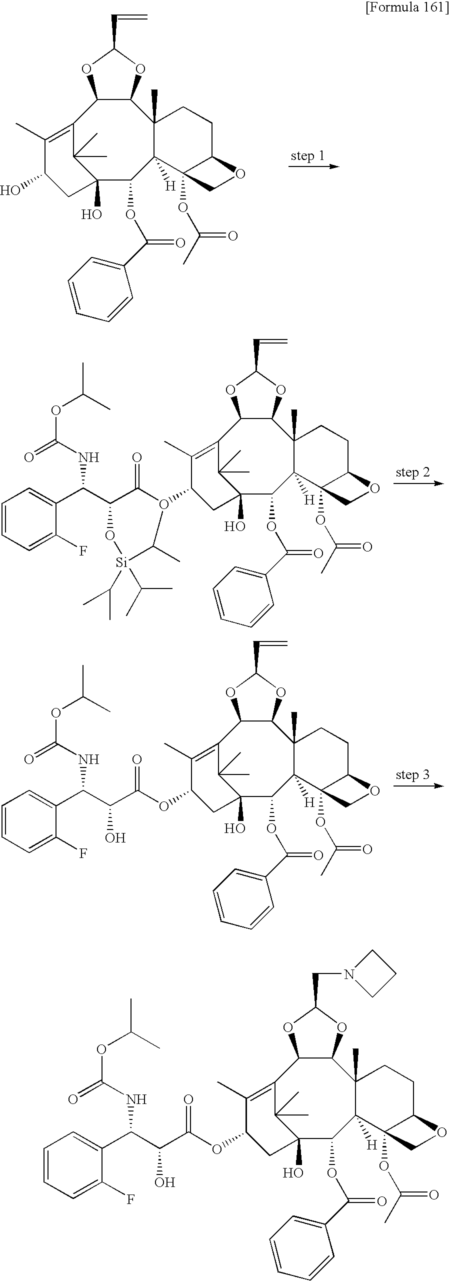

### Step 1: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3S)-3-(2-fluorophenyl)-3-(isopropoxycarbonylamino)-2-[(triisopropylsilyl)oxy]propionate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-11-ene and the compound obtained in Reference Example 42 were used as starting materials to perform the same procedure as that of Example 31, Step 1 and thereby to obtain the title compound as white amorphous solid.

¹H-NMR (400 MHz, CDCl₃) δ: 0.72-1.05 (21H, m), 1.06-2.33 (13H, m), 1.36 (3H, s), 1.52 (3H, s), 1.65 (3H, s), 1.80 (3H, s), 2.47 (3H, s), 2.96 (1H, d, J=5.1 Hz), 4.21-4.23 (2H, m), 4.35 (1H, d, J=8.3 Hz), 4.78-4.85 (2H, m), 4.94-4.95 (2H, m), 5.20 (1H, d, J=6.3 Hz), 5.26 (1H, d, J=7.1 Hz), 5.44-5.60 (4H, m), 5.96-6.04 (2H, m), 6.15 (1H, t, J=8.8 Hz), 7.05-7.09 (1H, m), 7.12-7.16 (1H, m), 7.21-7.33 (2H, m), 7.48 (2H, t, J=7.3 Hz), 7.57-7.61 (1H, m), 8.14-8.16 (2H, m).

FAB-MS; m/z: 992 (M+H)⁺, 1041 (M+Na)⁺.

### Step 2: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3S)-3-(2-fluorophenyl)-2-hydroxy-3-(isopropoxycarbonylamino)propionate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 0.91-2.10 (7H, m), 1.20 (6H, d, J=6.1 Hz), 1.27 (3H, s), 1.49 (3H, s), 1.64 (3H, s), 1.77 (3H, s), 2.28 (3H, s), 2.36 (1H, dd, J=15.0, 9.9 Hz), 2.93 (1H, d, J=4.6 Hz), 4.17 (1H, d, J=6.8 Hz), 4.25 (1H, d, J=8.3 Hz), 4.32 (1H, d, J=8.3 Hz), 4.63 (1H, s), 4.86-4.98 (2H, m), 5.25 (1H, d, J=6.1 Hz), 5.32 (1H, d, J=6.3 Hz), 5.46 (1H, d, J=10.3 Hz), 5.58 (1H, d, J=17.1 Hz), 5.71 (2H, brs), 5.97-6.05 (2H, m), 6.17 (1H, t, J=8.2 Hz), 7.08 (1H, t, J=9.4 Hz), 7.16 (1H, t, J=7.6 Hz), 7.26-7.32 (1H, m), 7.40-7.52 (3H, m), 7.60 (1H, t, J=7.4 Hz), 8.12 (2H, d, J=8.3 Hz).

FAB-MS; m/z: 836 (M+H)⁺, 858 (M+Na)⁺.

### Step 3: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3S)-3-(2-fluorophenyl)-2-hydroxy-3-(isopropoxycarbonylamino)propionate

The compound obtained in Step 2 mentioned above was used as a starting material to perform the same procedure as that of Example 1 and thereby to obtain the title compound as white amorphous solid.

¹H-NMR (400 MHz, CDCl₃) δ: 1.00-2.16 (9H, m), 1.20 (6H, d, J=6.3 Hz), 1.25 (3H, s), 1.48 (3H, s), 1.61 (3H, s), 1.75 (3H, s), 2.28 (3H, s), 2.35 (1H, dd, J=15.1, 9.8 Hz), 2.74 (1H, dd, J=12.4, 5.7 Hz), 2.83 (1H, dd, J=12.4, 4.4 Hz), 2.92 (1H, d, J=4.9 Hz), 3.32-3.40 (4H, m), 4.10 (1H, d, J=7.1 Hz), 4.24 (1H, d, J=8.5 Hz), 4.31 (1H, d, J=8.5 Hz), 4.63 (1H, s), 4.87 (1H, dd, J=5.6, 4.6 Hz), 4.91 (2H, s), 5.22 (1H, d, J=6.8 Hz), 5.61-5.75 (2H, m), 6.00 (1H, d, J=4.9 Hz), 6.15 (1H, t, J=8.0 Hz), 7.05-7.10 (1H, m), 7.16 (1H, t, J=7.6 Hz), 7.27-7.32 (1H, m), 7.37-7.48 (3H, m), 7.60 (1H, t, J=7.3 Hz), 8.11-8.13 (2H, m).

FAB-MS; m/z: 879 (M+H)⁺.

### Example 90

### (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-11-en-13-yl

### (2R,3S)-3-(2-fluorophenyl)-2-hydroxy-3-(isopropoxycarbonylamino)propionate

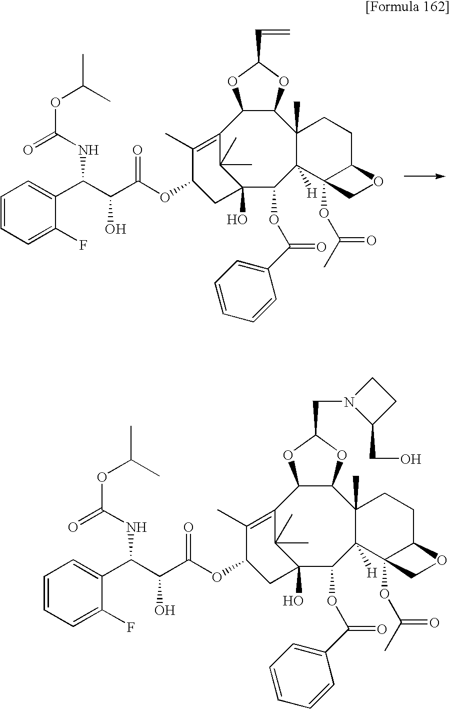

The compound obtained in Example 89, Step 2 and the compound obtained in Reference Example 25 were used as starting materials to perform the same procedure as that of Example 1 and thereby to obtain the title compound as white amorphous solid.

¹H-NMR (400 MHz, CDCl₃) δ: 0.88-2.05 (9H, m), 1.20 (6H, d, J=6.1 Hz), 1.25 (3H, s), 1.48 (3H, s), 1.59 (3H, s), 1.75 (3H, s), 2.20-2.38 (2H, m), 2.28 (3H, s), 2.83 (1H, dd, J=12.9, 4.6 Hz), 2.88-2.93 (2H, m), 3.11 (1H, dd, J=16.2, 8.7 Hz), 3.43 (1H, dd, J=11.5, 2.6 Hz), 3.46-3.52 (2H, m), 3.66 (1H, dd, J=11.5, 2.9 Hz), 4.12 (1H, d, J=7.3 Hz), 4.24 (1H, d, J=8.3 Hz), 4.31 (1H, d, J=8.3 Hz), 4.63 (1H, s), 4.86-4.91 (3H, m), 5.24 (1H, d, J=7.1 Hz), 5.63-5.76 (2H, m), 5.99 (1H, d, J=5.1 Hz), 6.14 (1H, t, J=8.3 Hz), 7.05-7.10 (1H, m), 7.16 (1H, td, J=7.6, 1.0 Hz), 7.27-7.32 (1H, m), 7.38-7.48 (3H, m), 7.58-7.62 (1H, m), 8.10-8.13 (2H, m).

FAB-MS; m/z: 909 (M+H)⁺.

### Example 91

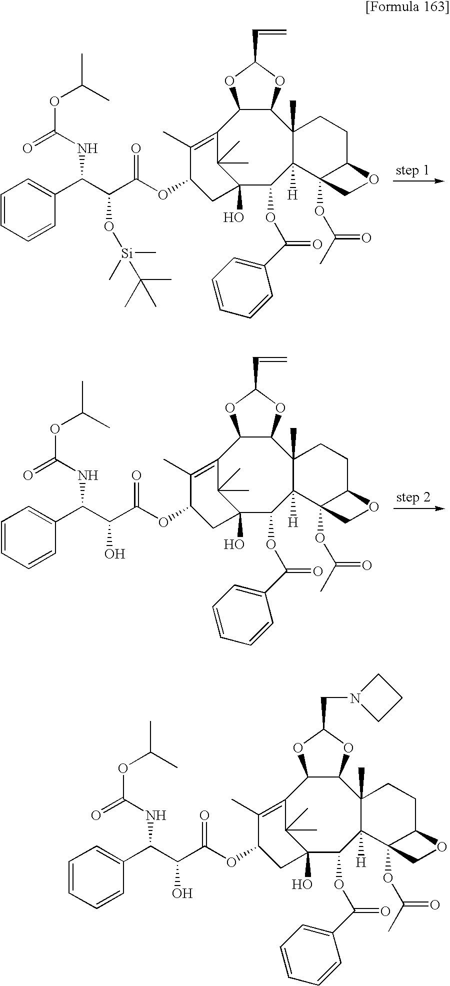

### Step 1: (1S,2S,3R,4S,5R,8R,9S,10R,13S)-4-Acetoxy-2-(benzoyloxy)-5,20-epoxy-1-hydroxy-9,10-[(1S)-2-propenylidenedioxy]tax-11-en-13-yl

### (2R,3S)-2-hydroxy-3-(isopropoxycarbonylamino)-3-phenylpropionate

The compound obtained in Example 74, Step 1 was used as a starting material to perform the same procedure as that of Example 31, Step 2 and thereby to obtain the title compound as white amorphous solid.

¹H-NMR (400 MHz, CDCl₃) δ: 0.86-2.10 (6H, m), 1.20 (6H, d, J=6.9 Hz), 1.25 (3H, s), 1.48 (3H, s), 1.60 (3H, s), 1.63 (3H, s), 2.27 (3H, s), 2.33-2.40 (1H, m), 2.91 (1H, d, J=5.2 Hz), 4.15 (1H, d, J=6.9 Hz), 4.24 (1H, d, J=8.6 Hz), 4.32 (1H, d, J=8.6 Hz), 4.62 (2H, d, J=9.6 Hz), 4.85-4.92 (2H, m), 5.24 (1H, d, J=6.1 Hz), 5.27-5.30 (1H, m), 5.37 (1H, d, J=9.3 Hz), 5.46 (1H, d, J=10.8 Hz), 5.62 (1H, t, J=25.6 Hz), 5.81 (1H, d, J=9.8 Hz), 5.96-6.05 (2H, m), 6.10 (1H, t, J=8.6 Hz), 7.27-7.32 (1H, m), 7.34-7.38 (2H, m), 7.41-7.49 (4H, m), 7.55-7.62 (1H, m), 8.12 (2H, dd, J=8.1, 1.2 Hz).

### Step 2: (1S,2S,3R,4S,5R,8R,9S,10R,13S)-4-Acetoxy-9,10-[(1S)-2-(azetidino)ethylidenedioxy]-2-(benzoyloxy)-5,20-epoxy-1-hydroxytax-11-en-13-yl

### (2R,3S)-2-hydroxy-3-(isopropoxycarbonylamino)-3-phenylpropionate

The compound obtained in Step 1 mentioned above was used as a starting material to perform the same procedure as that of Example 1 and thereby to obtain the title compound as white amorphous solid.

¹H-NMR (400 MHz, CDCl₃) δ: 1.20 (6H, d, J=6.1 Hz), 1.24 (3H, s), 1.47 (3H, s), 1.59-2.17 (15H, m), 2.27 (3H, s), 2.35 (1H, d, J=15.2, 10.1 Hz), 2.61-2.90 (3H, m), 3.30-3.43 (4H, m), 4.08 (1H, d, J=7.1 Hz), 4.24 (1H, d, J=8.6 Hz), 4.31 (1H, d, J=8.6 Hz), 4.63 (1H, s), 4.84-4.92 (3H, m), 5.19 (1H, d, J=6.9 Hz), 5.36 (1H, d, J=9.1H), 5.81 (1H, d, J=9.1 Hz), 6.00 (1H, d, J=4.9 Hz), 6.08 (1H, t, J=8.3 Hz), 7.27-7.37 (3H, m), 7.41-7.49 (4H, m), 7.58-7.62 (1H, m), 8.12 (2H, dd, J=8.3, 1.2 Hz).

FAB-MS; m/z: 861 (M+H)⁺.

### Example 92

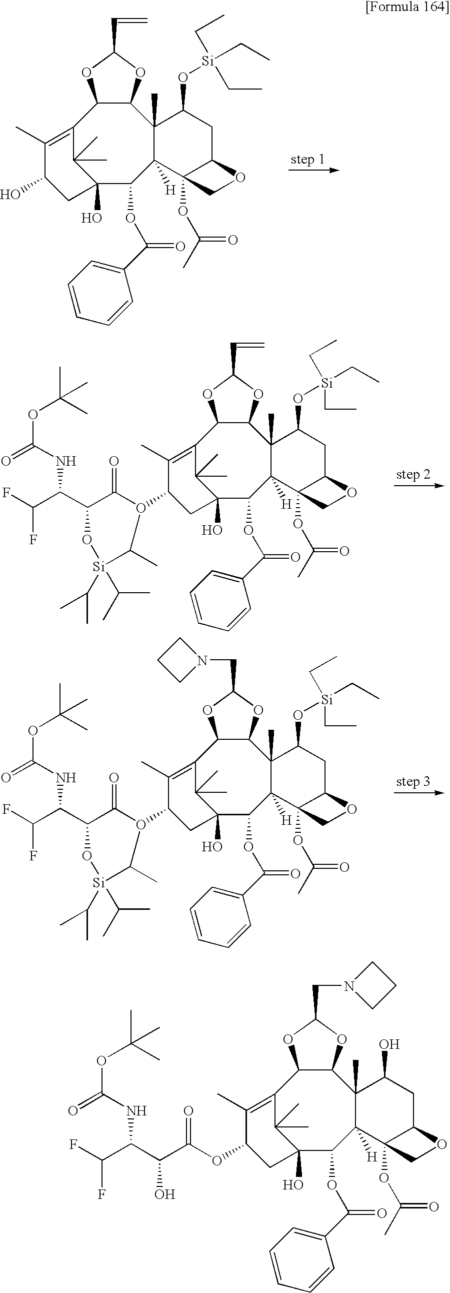

### Step 1: (1S,2S,3R,4S,5R,7S,8R,9S,10R,13S)-4-Acetoxy-2-(benzoyloxy)-5,20-epoxy-1-hydroxy-9,10-[(1S)-2-propenylidenedioxy]-7-[(triethylsilyl)oxy]tax-11-en-13-yl

### (2R,3R)-3-(tert-butoxycarbonylamino)-4,4-difluoro-2-[(triisopropylsilyl)oxy]butyrate

(1S,2S,3R,4S,5R,7S,8R,9S,10R,13S)-4-(Acetoxy)-2-(benzoyloxy)-1,13-dihydroxy-5,20-epoxy-9,10-[(1S)-2-propenylidenedioxy]-7-[(triethylsilyl)oxy]tax-11-ene and (3R,4R)-1-(tert-butoxycarbonyl)-4-(difluoromethyl)-3-[(triisopropylsilyl)oxy]-2-azetidin one were used as starting materials to perform the same procedure as that of Example 31, Step 1 and thereby to obtain the title compound as white amorphous solid.

¹H-NMR (400 MHz, CDCl₃) δ: 0.60-0.67 (6H, m), 0.87-1.76 (4H, m), 0.99 (9H, t, J=8.1 Hz), 1.10-1.12 (21H, m), 1.19 (3H, s), 1.37 (9H, s), 1.59 (3H, s), 1.75 (3H, s), 2.03-2.10 (1H, m), 2.20-2.26 (1H, m), 2.32-2.42 (2H, m), 2.34 (3H, s), 3.17 (1H, d, J=5.4 Hz), 3.92 (1H, dd, J=10.2, 5.5 Hz), 4.32-4.40 (3H, m), 4.64 (1H, d, J=8.8 Hz), 4.80-4.85 (2H, m), 5.02 (1H, d, J=10.3 Hz), 5.06 (1H, d, J=5.9 Hz), 5.28 (1H, d, J=8.8 Hz), 5.46 (1H, d, J=10.8 Hz), 5.56 (1H, d, J=17.7 Hz), 5.78 (1H, td, Jt=56.1, Jd=6.1 Hz), 5.90 (1H, d, J=5.6 Hz), 6.05 (1H, t, J=9.3 Hz), 6.10-6.18 (1H, m), 7.50 (2H, t, J=7.6 Hz), 7.59 (1H, tt, J=7.5, 1.6 Hz), 8.11 (2H, d, J=7.1 Hz).

### Step 2: (1S,2S,3R,4S,5R,7S,8R,9S,10R,13S)-4-Acetoxy-9,10-[(1S)-2-(azetidino)ethylidenedioxy]-2-(benzoyloxy)-5,20-epoxy-1-hydroxy-7-[(triethylsilyl)oxy]tax-11-en-13-yl

### (2R,3R)-3-(tert-butoxycarbonylamino)-4,4-difluoro-2-[(triisopropylsilyl)oxy]butyrate

The compound obtained in Step 1 mentioned above was used as a starting material to perform the same procedure as that of Example 1 and thereby to obtain the title compound as white amorphous solid.

¹H-NMR (400 MHz, CDCl₃) δ: 0.58-0.66 (6H, m), 0.93-2.25 (7H, m), 0.97 (9H, t, J=7.8 Hz), 1.09-1.12 (21H, m), 1.19 (3H, s), 1.36 (9H, s), 1.51 (3H, s), 1.73 (3H, s), 1.88 (1H, brs), 2.30-2.39 (2H, m), 2.34 (3H, s), 2.85 (2H, d, J=4.9 Hz), 3.15 (1H, d, J=5.4 Hz), 3.33 (4H, t, J=7.0 Hz), 3.89 (1H, dd, J=10.3, 5.4 Hz), 4.30-4.40 (3H, m), 4.49 (1H, d, J=8.8 Hz), 4.67 (1H, t, J=5.0 Hz), 4.79-4.84 (2H, m), 5.02 (1H, d, J=10.3 Hz), 5.18 (1H, d, J=9.1 Hz), 5.78 (1H, td, Jt=55.9, Jd=6.1 Hz), 5.88 (1H, d, J=5.6 Hz), 6.05 (1H, t; J=8.8 Hz), 7.50 (2H, t, J=7.6 Hz), 7.59 (1H, t, J=7.4 Hz), 8.11 (2H, d, J=7.1 Hz).

### Step 3: (1S,2S,3R,4S,5R,7S,8S,9S,10R,13S)-4-Acetoxy-9,10-[(1S)-2-(azetidino)ethylidenedioxy]-2-(benzoyloxy)-1,7-dihydroxy-5,20-epoxytax-11-en-13-yl

### (2R,3R)-3-(tert-butoxycarbonylamino)-4,4-difluoro-2-hydroxybutyrate

The compound obtained in Step 2 mentioned above was used as a starting material to perform the same procedure as that of Example 43, Step 3 and thereby to obtain the title compound as white amorphous solid.

¹H-NMR (400 MHz, CDCl₃) δ: 1.25 (3H, s), 1.39-1.46 (1H, m), 1.41 (9H, s), 1.62 (3H, s), 1.66 (3H, s), 1.72 (3H, s), 1.89 (1H, s), 2.04-2.39 (7H, m), 2.36 (3H, s), 2.79-2.95 (3H, m), 3.33-3.46 (4H, m), 3.81 (1H, d, J=7.11 Hz), 4.08 (1H, brs), 4.33 (1H, d, J=8.8 Hz), 4.40 (1H, d, J=8.6 Hz), 4.45-4.56 (1H, m), 4.63 (1H, s), 4.87-4.90 (1H, m), 5.11 (1H, s), 5.17-5.23 (2H, m), 5.89 (1H, td, Jt=56.1, Jd=5.2 Hz), 6.06 (1H, d, J=4.9 Hz), 6.11 (1H, t, J=8.8 Hz), 7.49 (2H, t, J=7.7 Hz), 7.59-7.64 (1H, m), 8.11-8.13 (2H, m).

FAB-MS; m/z: 865 (M+H)+

### Example 93

### (1S,2S,3R,4S,5R,8R,9S,10R,13S)-4-(Acetoxy)-9,10-{(1S)-2-[(2S)-2-(dimethylcarbamoyl) azetidino]ethylidenedioxy}-2-(benzoyloxy)-5,20-epoxy-1-hydroxytax-11-en-13-yl

### (2R,3R)-3-(tert-butoxycarbonylamino)-4,4-difluoro-2-hydroxybutyrate

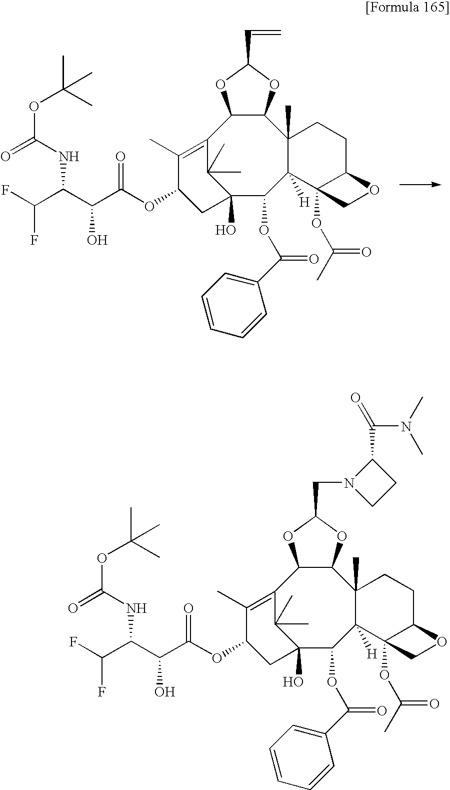

The compound obtained in Example 31, Step 2 and the compound obtained in Reference Example 12, Step 2 were used as starting materials to perform the same procedure as that of Example 1 and thereby to obtain the title compound as white amorphous solid.

¹H-NMR (400 MHz, CDCl₃) δ: 1.16-2.37 (9H, m), 1.18 (3H, s), 1.44 (9H, s), 1.48 (3H, s), 1.58 (3H, s), 1.75 (3H, s), 2.33 (3H, s), 2.82-3.01 (3H, m), 2.92 (3H, s), 2.94 (3H, s), 3.09 (1H, q, J=8.6 Hz), 3.54-3.60 (1H, m), 3.95 (1H, t, J=8.5 Hz), 4.09 (1H, d, J=7.1 Hz), 4.24 (1H, d, J=8.3 Hz), 4.32 (1H, d, J=8.6 Hz), 4.48-4.59 (1H, brm), 4.65 (1H, s), 4.72-4.80 (1H, brm), 4.93 (1H, s), 5.07 (1H, t, J=3.9 Hz), 5.19 (1H, d, J=6.9 Hz), 5.28 (1H, d, J=10.0 Hz), 5.89 (1H, td, J=56.1, 4.7 Hz), 6.00 (1H, d, J=5.1 Hz), 6.08 (1H, t, J=7.8 Hz), 7.48 (2H, t, J=7.7 Hz), 7.61 (1H, t, J=7.5 Hz), 8.13 (2H, d, J=7.4 Hz).

FAB-MS; m/z: 921 (M+H)⁺.

### Example 94

### (1S,2S,3R,4S,5R,8R,9S,10R,13S)-4-(Acetoxy)-2-(benzoyloxy)-9,10-{(1S)-2-[3-(dimethylcarbamoyl)azetidino]ethylidenedioxy}-5,20-epoxy-1-hydroxytax-11-en-13-yl

### (2R,3R)-3-(tert-butoxycarbonylamino)-4,4-difluoro-2-hydroxybutyrate

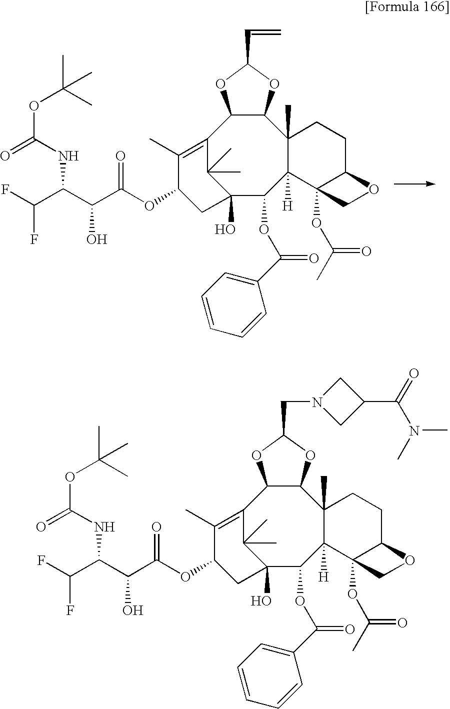

The compound obtained in Example 31, Step 2 and the compound obtained in Reference Example 2, Step 2 were used as starting materials to perform the same procedure as that of Example 1 and thereby to obtain the title compound as white amorphous solid.

¹H-NMR (400 MHz, CDCl₃) δ: 1.15-2.39 (9H, m), 1.23 (3H, s), 1.42 (9H, s), 1.48 (3H, s), 1.60 (3H, s), 1.77 (3H, s), 2.33 (3H, s), 2.79 (1H, dd, J=12.5, 5.6 Hz), 2.86 (1H, dd, J=12.5, 4.4 Hz), 2.91 (3H, s), 2.96 (3H, s), 3.39-3.49 (2H, m), 3.50-3.58 (1H, m), 3.68-3.75 (2H, m), 4.11 (1H, d, J=7.1 Hz), 4.24 (1H, d, J=8.6 Hz), 4.33 (1H, d, J=8.6 Hz), 4.45-4.60 (1H, brm), 4.64 (1H, s), 4.88 (1H, t, J=4.9 Hz), 4.92 (1H, s), 5.22 (1H, d, J=6.9 Hz), 5.26 (1H, d, J=9.8 Hz), 5.90 (1H, td, J=55.9, 4.9 Hz), 5.99 (1H, d, J=4.9 Hz), 6.11 (1H, t, J=7.8 Hz), 7.48 (2H, t, J=7.7 Hz), 7.61 (1H, t, J=7.5 Hz), 8.13 (2H, d, J=7.4 Hz).

FAB-MS; m/z: 921 (M+H)⁺.

### Example 95

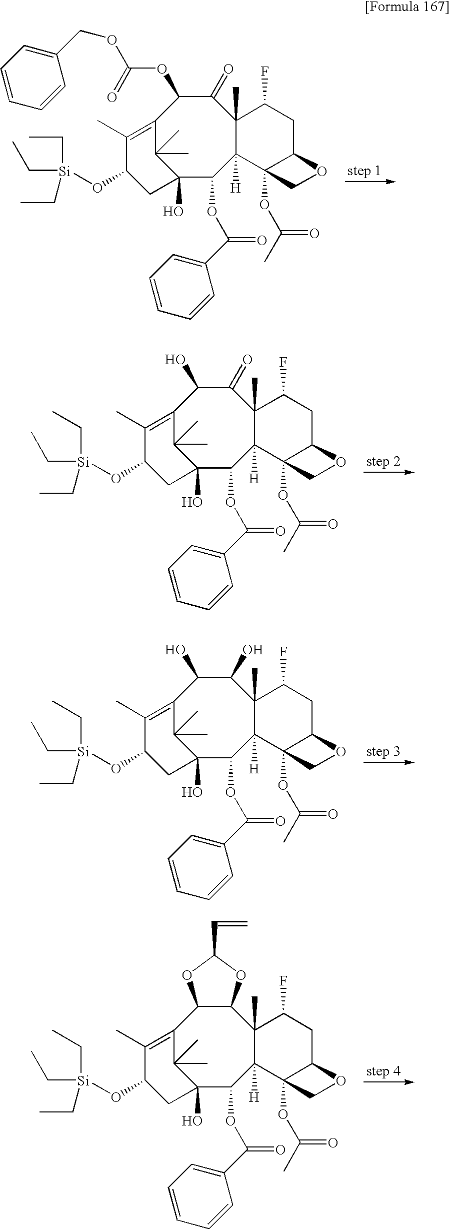

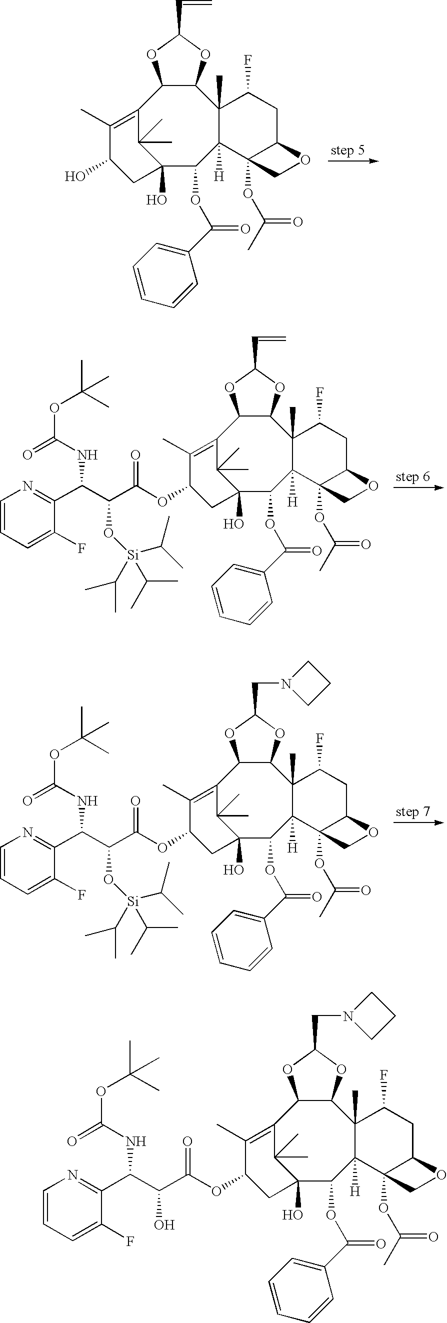

### Step 1: (1S,2S,3R,4S,5R,7R,8R,10R,13S)-4-(Acetoxy)-2-(benzoyloxy)-1,10-dihydroxy-5,20-epoxy-7-fluoro-9-oxo-13-[(triethylsilyl)oxy]tax-11-ene

(1S,2S,3R,4S,5R,7R,8R,10R,13S)-4-(Acetoxy)-2-(benzoyloxy)-10-[(benzyloxycarbonyl)oxy]-5,20-epoxy-7-fluoro-1-hydroxy-9-oxo-13-[(triethylsilyl)oxy]tax-11-ene (Bioorg. Med. Chem. Lett., 2002, 12, 2815, 1.25 g) was dissolved in a mixed solvent of ethanol (20 ml) and tetrahydrofuran (10 ml), and the solution was added with 10% palladium/carbon (0.3 g), and stirred for 1 hour under a hydrogen atmosphere. The catalyst was removed by filtration, and then the filtrate was concentrated under reduced pressure. The resulting residue was purified by silica gel chromatography [developing solvent; ethyl acetate:n-hexane=1:2 (v/v)] to obtain the title compound (1.02 g) as colorless solid.

¹H-NMR (400 MHz, CDCl₃) δ: 0.60-0.72 (6H, m), 1.01 (9H, t, J=7.8 Hz), 1.09 (3H, s), 1.17 (3H, s), 1.77 (3H, s), 1.96 (3H, s), 2.11-2.36 (3H, m), 2.30 (3H, s), 2.52-2.65 (1H, m), 4.05 (1H, d, J=7.2 Hz), 4.06 (1H, s), 4.28 (1H, d, J=8.3 Hz), 4.37 (1H, d, J=8.3 Hz), 4.55 (1H, dd, J=47.4, 4.2 Hz), 4.95 (1H, t, J=8.8 Hz), 5.05 (1H, d, J=8.5 Hz), 5.27 (1H, s), 5.71 (1H, d, J=7.2 Hz), 6.41 (1H, s), 7.49 (2H, t, J=7.8 Hz), 7.60 (1H, t, J=7.8 Hz), 8.12 (2H, d, J=7.8 Hz).

### Step 2: (1S,2S,3R,4S,5R,7R,8R,9S,10R,13S)-4-(Acetoxy)-2-(benzoyloxy)-5,20-epoxy-7-fluoro-13-[(triethylsilyl)oxy]-1,9,10-trihydroxytax-11-ene

The compound obtained in Step 1 mentioned above (1.00 g) was dissolved in tetrahydrofuran (20 ml), and the solution was cooled on ice. Then, the solution was added dropwise with borane/tetrahydrofuran complex (1 mole solution in tetrahydrofuran, 45 ml), and stirred at the same temperature for 4 hours. The reaction mixture was added with saturated aqueous sodium hydrogencarbonate, and extracted with ethyl acetate, and the organic layer was dried over anhydrous sodium sulfate. The solvent was evaporated under reduced pressure, and then the resulting residue was purified by silica gel chromatography [developing solvent; ethyl acetate:n-hexane=1:2 (v/v)] to obtain the title compound (270 mg) as colorless solid.

¹H-NMR (400 MHz, CDCl₃) δ: 0.59-0.72 (6H, m), 1.02 (9H, t, J=7.8 Hz), 1.26 (3H, s), 1.62 (3H, s), 1.73 (3H, s), 1.84 (3H, d, J=3.4 Hz), 2.19-2.28 (3H, m), 2.26 (3H, s), 2.47-2.55 (1H, m), 3.23 (1H, d, J=2.2 Hz), 3.45 (1H, d, J=5.1 Hz), 4.11-4.14 (1H, m), 4.15 (1H, d, J=8.3 Hz), 4.38 (1H, d, J=8.3 Hz), 4.74 (1H, dd, J=47.6, 4.4 Hz), 5.01 (1H, t, J=8.3 Hz), 5.03 (1H, d, J=8.3 Hz), 5.33 (1H, br), 5.99 (1H, d, J=7.3 Hz), 7.47 (2H, t, J=8.3 Hz), 7.56 (1H, t, J=8.3 Hz), 8.11 (2H, d, J=8.3 Hz).

### Step 3: (1S,2S,3R,4S,5R,7R,8R,9S,10R,13S)-4-(Acetoxy)-2-(benzoyloxy)-5,20-epoxy-7-fluoro-1-hydroxy-9,10-[(1S)-2-propenylidenedioxy]-13-[(triethylsilyl)oxy]tax-11-ene

The compound obtained in Step 2 mentioned above and 3,3-diethoxy-1-propene were used as starting materials to perform the same procedure as that of Example 81, Step 1 and thereby to obtain the title compound as colorless solid.

¹H-NMR (400 MHz, CDCl₃) δ: 0.59-0.73 (6H, m), 1.01 (9H, t, J=7.8 Hz), 1.17 (3H, s), 1.53 (3H, s), 1.62 (3H, s), 1.74 (1H, s), 1.88 (3H, s), 2.06-2.26 (3H, m), 2.27 (3H, s), 2.47-2.58 (1H, m), 3.47 (1H, d, J=5.6 Hz), 4.27 (1H, d, J=8.5 Hz), 4.37 (1H, d, J=8.8 Hz), 4.38 (1H, d, J=8.5 Hz), 4.79 (1H, dt, J=45.5, 5.0 Hz), 4.96 (1H, brt, J=8.8 Hz), 5.00 (1H, dd, J=7.0, 3.2 Hz), 5.12 (1H, d, J=5.9 Hz), 5.43 (1H, d, J=8.8 Hz), 5.47 (1H, d, J=10.5 Hz), 5.59 (1H, d, J=17.1 Hz), 5.85 (1H, d, J=5.6 Hz), 6.11 (1H, ddd, J=17.1, 10.5, 5.9 Hz), 7.48 (2H, t, J=8.3 Hz), 7.59 (1H, t, J=8.3 Hz), 8.11 (2H, d, J=8.3 Hz).

### Step 4: (1S,2S,3R,4S,5R,7R,8R,9S,10R,13S)-4-(Acetoxy)-2-(benzoyloxy)-1,13-dihydroxy-5,20-epoxy-7-fluoro-9,10-[(1S)-2-propenylidenedioxy]tax-11-ene

The compound obtained in Step 3 mentioned above was used as a starting material to perform the same procedure as that of Example 84, Step 4 and thereby to obtain the title compound as colorless solid.

¹H-NMR (400 MHz, CDCl₃) δ: 1.08 (3H, s), 1.57 (3H, s), 1.64 (3H, s), 1.73 (1H, s), 1.93 (3H, s), 2.08-2.36 (3H, m), 2.32 (3H, s), 2.43-2.55 (2H, m), 3.58 (1H, d, J=5.3 Hz), 4.27 (1H, d, J=7.8 Hz), 4.28 (1H, d, J=8.5 Hz), 4.40 (1H, d, J=7.8 Hz), 4.78 (1H, dt, J=45.8, 4.9 Hz), 4.74-4.79 (1H, m), 4.94 (1H, d, J=4.9 Hz), 5.13 (1H, d, J=6.1 Hz), 5.45 (1H, d, J=8.5 Hz), 5.49 (1H, d, J=10.5 Hz), 5.59 (1H, d, J=17.2 Hz), 5.90 (1H, d, J=5.3 Hz), 6.14 (1H, ddd, J=17.2, 10.5, 6.1 Hz), 7.49 (2H, t, J=7.8 Hz), 7.61 (1H, t, J=7.8 Hz), 8.12 (2H, d, J=7.8 Hz).

### Step 5: (1S,2S,3R,4S,5R,7R,8R,9S,10R,13S)-4-(Acetoxy)-2-(benzoyloxy)-1-hydroxy-5,20-epoxy-7-fluoro-9,10-[(1S)-2-propenylidenedioxy]tax-1′-en-13-yl

### (2R,3S)-3-(tert-butoxycarbonylamino)-3-(3-fluoro-2-pyridyl)-2-[(triisopropylsilyl)oxy]propionate

The compound obtained in Step 4 mentioned above and (3R,4S)-1-(tert-butoxycarbonyl)-4-(3-fluoro-2-pyridyl)-3-[(triisopropylsilyl)oxy]-2-azetidinone were used as starting materials to perform the same procedure as that of Example 31, Step 1 and thereby to obtain the title compound as white amorphous solid.

¹H-NMR (400 MHz, CDCl₃) δ: 0.82-0.95 (21H, m), 1.28 (3H, s), 1.38 (9H, s), 1.51-2.54 (5H, m), 1.57 (3H, s), 1.65 (3H, s), 1.80 (3H, s), 2.50 (3H, s), 3.52 (1H, d, J=5.6 Hz), 4.34 (1H, d, J=8.3 Hz), 4.36-4.42 (2H, m), 4.73-4.90 (1H, m), 4.95 (1H, brs), 4.97-5.02 (1H, m), 5.12 (1H, d, J=5.9 Hz), 5.41 (1H, d, J=8.1 Hz), 5.47 (1H, d, J=11.0 Hz), 5.56-5.64 (2H, m), 5.93 (1H, d, J=5.4 Hz), 6.01-6.18 (3H, m), 7.24-7.29 (1H, m), 7.40 (1H, t, J=9.2 Hz), 7.48 (2H, t, J=7.1 Hz), 7.58 (1H, t, J=7.1 Hz), 8.14 (2H, d, J=8.3 Hz), 8.40 (1H, d, J=3.9 Hz).

FAB-MS; m/z: 1025 (M+H)⁺.

### Step 6: (1S,2S,3R,4S,5R,7R,8R,9S,10R,13S)-4-(Acetoxy)-9,10-[(1S)-2-(azetidino)ethylidenedioxy]-2-(benzoyloxy)-5,20-epoxy-7-fluoro-1-hydroxytax-11-en-13-yl

### (2R,3S)-3-(tert-butoxycarbonylamino)-3-(3-fluoro-2-pyridyl)-2-[(triisopropylsilyl)oxy]propionate

The compound obtained in Step 5 mentioned above was used as a starting material to perform the same procedure as that of Example 1 and thereby to obtain the title compound as white amorphous solid.

¹H-NMR (400 MHz, CDCl₃) δ: 0.82-0.93 (21H, m), 1.27 (3H, s), 1.37 (9H, s), 1.54 (3H, s), 1.62 (3H, s), 1.64-2.48 (7H, m), 1.78 (3H, s), 2.50 (3H, s), 2.81-2.91 (2H, m), 3.35 (4H, t, J=7.0 Hz), 3.49 (1H, d, J=5.6 Hz), 4.29-4.36 (2H, m), 4.39 (1H, d, J=9.0 Hz), 4.70-4.88 (1H, m), 4.73 (1H, t, J=5.1 Hz), 4.94 (1H, d, J=2.4 Hz), 4.98 (1H, brs), 5.31 (1H, d, J=8.8 Hz), 5.61 (1H, d, J=9.8 Hz), 5.90 (1H, d, J=5.6 Hz), 6.06 (1H, d, J=10.5 Hz), 6.12 (1H, t, J=9.0 Hz), 7.24-7.29 (1H, m), 7.40 (1H, t, J=9.1 Hz), 7.48 (2H, t, J=7.6 Hz), 7.59 (1H, t, J=7.6 Hz), 8.14 (2H, d, J=8.5 Hz), 8.40 (1H, d, J=4.4 Hz).

FAB-MS; m/z: 1068 (M+H)⁺.

### Step 7: (1S,2S,3R,4S,5R,7R,8R,9S,10R,13S)-4-(Acetoxy)-9,10-[(1S)-2-(azetidino)ethylidenedioxy]-2-(benzoyloxy)-5,20-epoxy-7-fluoro-1-hydroxytax-1′-en-13-yl

### (2R,3S)-3-(tert-butoxycarbonylamino)-3-(3-fluoro-2-pyridyl)-2-hydroxypropionate

The compound obtained in Step 6 mentioned above was used as a starting material to perform the same procedure as that of Example 31, Step 2 and thereby to obtain the title compound as white amorphous solid.

¹H-NMR (400 MHz, CDCl₃) δ: 1.22 (3H, s), 1.39 (9H, s), 1.53-2.55 (8H, m), 1.57 (3H, s), 1.62 (3H, s), 1.77 (3H, s), 2.36 (3H, s), 2.83-2.93 (2H, m), 3.35 (4H, t, J=7.1 Hz), 3.48 (1H, d, J=5.1 Hz), 4.24 (1H, d, J=9.0 Hz), 4.28 (1H, d, J=8.8 Hz), 4.38 (1H, d, J=88.5 Hz), 4.65 (1H, d, J=2.7 Hz), 4.68-4.84 (1H, m), 4.73 (1H, t, J=4.6 Hz), 4.95 (1H, d, J=6.3 Hz), 5.36 (1H, d, J=8.8 Hz), 5.64 (1H, d, J=8.5 Hz), 5.91 (1H, d, J=5.4 Hz), 6.09 (1H, t, J=8.5 Hz), 6.18 (1H, d, J=8.0 Hz), 7.27-7.33 (1H, m), 7.42-7.52 (3H, m), 7.60 (1H, t, J=6.8 Hz), 8.11 (2H, d, J=9.0 Hz), 8.40 (1H, d, J=4.6 Hz).

FAB-MS; m/z: 912 (M+H)⁺.

### Example 96

### (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3R)-4,4-difluoro-2-hydroxy-3-(isopropoxycarbonylamino)pentanoate

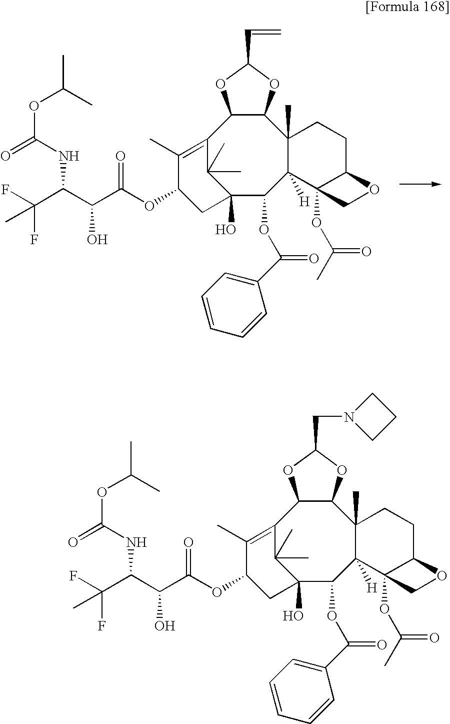

The compound obtained in Example 73, Step 2 was used as a starting material to perform the same procedure as that of Example 1 and thereby to obtain the title compound as white amorphous solid.

¹H-NMR (400 MHz, CDCl₃) δ: 1.16-1.27 (6H, m), 1.17-2.37 (8H, m), 1.23 (3H, s), 1.48 (3H, s), 1.61 (3H, s), 1.72 (3H, t, J=19.1 Hz), 1.73 (3H, s), 2.34 (3H, s), 2.75 (1H, dd, J=12.5, 5.9 Hz), 2.83 (1H, dd, J=12.5, 4.2 Hz), 2.92 (1H, d, J=4.9 Hz), 3.36 (4H, dd, J=11.0, 6.9 Hz), 4.10 (1H, d, J=6.9 Hz), 4.25 (1H, d, J=8.6 Hz), 4.34 (1H, d, J=8.6 Hz), 4.57-4.68 (1H, m), 4.77 (1H, s), 4.88 (1H, t, J=5.1 Hz), 4.90-4.97 (3H, m), 4.93 (1H, s), 5.21 (1H, d, J=6.9 Hz), 5.44 (1H, d, J=10.3 Hz), 6.01 (1H, d, J=4.9 Hz), 6.15 (1H, t, J=7.8 Hz), 7.48 (2H, t, J=7.7 Hz), 7.58-7.64 (1H, m), 8.11-8.15 (2H, m).

FAB-MS; m/z: 849 (M+H)⁺.

### Example 97

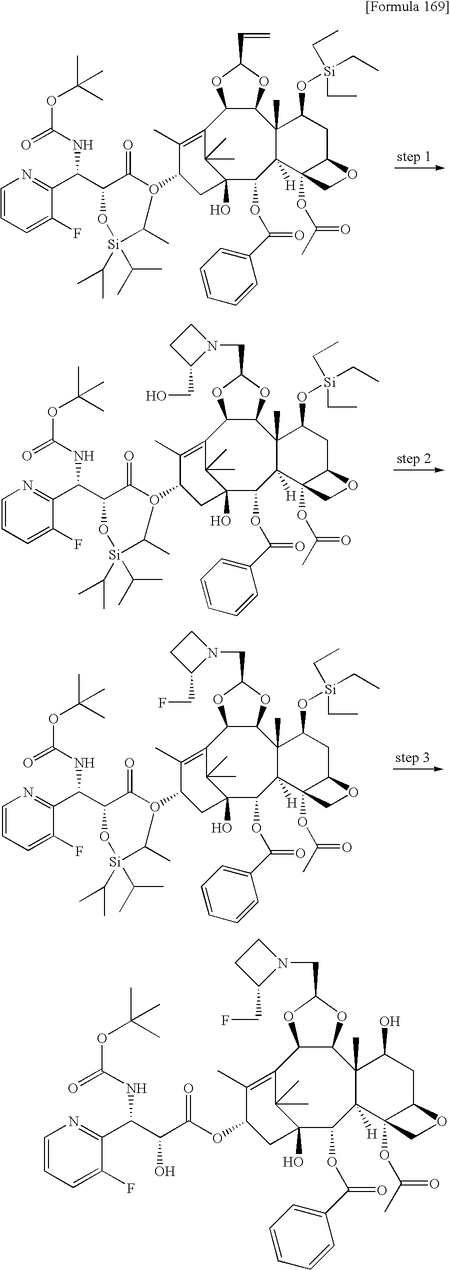

### Step 1: (1S,2S,3R,4S,5R,7S,8R,9S,10R,13S)-4-Acetoxy-2-(benzoyloxy)-5,20-epoxy-1-hydroxy-9,10-{(1S)-2-[(2S)-hydroxymethylazetidino]ethylidenedioxy}-7-[(triethylsilyl)oxy]tax-11-en-13-yl

### (2R,3S)-3-(tert-butoxycarbonylamino)-3-(3-fluoro-2-pyridyl)-2-[(triisopropylsilyl)oxy]propionate

The compounds obtained in Example 43, Step 1 and Reference Example 25 were used as starting materials to perform the same procedure as that of Example 1 and thereby to obtain the title compound as white amorphous solid. The resultant was used for the subsequent reaction as it was as a crude product.

### Step 2: (1S,2S,3R,4S,5R,7S,8R,9S,10R,13S)-4-Acetoxy-2-(benzoyloxy)-5,20-epoxy-9,10-{(1S)-2-[(2S)-fluoromethylazetidino]ethylidenedioxy}-1-hydroxy-7-[(triethylsilyl)oxy]tax-11-en-13-yl

### (2R,3S)-3-(tert-butoxycarbonylamino)-3-(3-fluoro-2-pyridyl)-2-[(triisopropylsilyl)oxy]propionate

The compound obtained in Step 1 mentioned above (396 mg) was dissolved in dichloromethane (10 ml), and the solution was added with diethylaminosulfur trifluoride (56 μl) at −78° C. under an argon atmosphere, stirred at −78° C. for 1 hour and at 0° C. for 1 hour, further added with diethylaminosulfur trifluoride (56 μl), further stirred at room temperature for 30 minutes, and then added with water under ice cooling, and the layers were separated. The organic layer was washed with saturated brine, and dried over anhydrous sodium sulfate, and the solvent was evaporated under reduced pressure. The resulting residue was purified by silica gel chromatography [ethyl acetate:n-hexane=1:2 (v/v)] to obtain the title compound as white solid (181 mg).

¹H-NMR (400 MHz, CDCl₃) δ: 0.60-0.68 (6H, m), 0.85-2.63 (14H, m), 0.86-0.94 (21H, m), 0.99 (9H, t, J=7.8 Hz), 1.26 (3H, s), 1.37 (9H, s), 1.52 (3H, s), 1.77 (3H, s), 2.48 (3H, s), 2.82-3.06 (3H, m), 3.24 (1H, d, J=5.6 Hz), 3.93 (1H, dd, J=10.8, 4.7 Hz), 4.34 (1H, d, J=8.1 Hz), 4.43-4.51 (2H, m), 4.81-4.88 (2H, m), 4.93 (1H, s), 5.20-5.27 (1H, m), 5.62 (1H, d, J=9.8 Hz), 5.89 (1H, d, J=5.4 Hz), 6.04 (1H, d, J=10.1 Hz), 6.12 (1H, t, J=9.3 Hz), 7.24-7.29 (1H, m), 7.40 (1H, t, J=9.3 Hz), 7.47 (2H, t, J=7.4 Hz), 7.54-7.59 (1H, m), 8.12 (2H, d, J=7.1 Hz), 8.40 (1H, d, J=4.2 Hz).

### Step 3: (1S,2S,3R,4S,5R,7S,8S,9S,10R,13S)-4-Acetoxy-2-(benzoyloxy)-1,7-dihydroxy-5,20-epoxy-9,10-{(1S)-2-[(2S)-fluoromethylazetidino]ethylidenedioxy}tax-11-en-13-yl

### (2R,3S)-3-(tert-butoxycarbonylamino)-3-(3-fluoro-2-pyridyl)-2-hydroxypropionate

The compound obtained in Step 2 mentioned above was used as a starting material to perform the same procedure as that of Example 31, Step 2 and thereby to obtain the title compound as white amorphous solid.

¹H-NMR (400 MHz, CDCl₃) δ: 1.32 (3H, s), 1.40 (9H, s), 1.62 (3H, s), 1.68 (3H, s), 1.76 (3H, s), 1.88-2.27 (6H, m), 2.39 (3H, s), 2.42-2.50 (1H, m), 2.60 (1H, q, J=7.5 Hz), 2.75-2.88 (2H, m), 2.94-3.15 (4H, m), 3.85 (1H, d, J=7.4 Hz), 3.91 (1H, brs), 4.05-4.11 (1H, m), 4.32 (1H, d, J=8.6 Hz), 4.40 (1H, d, J=8.6 Hz), 4.64-4.70 (2H, m), 5.03 (1H, dd, J=5.4, 3.9 Hz), 5.10-5.28 (3H, m), 5.63 (1H, d, J=8.6 Hz), 6.05 (1H, d, J=4.9 Hz), 6.11 (1H, t, J=8.6 Hz), 6.21 (1H, d, J=8.6 Hz), 7.29-7.34 (1H, m), 7.44-7.52 (3H, m), 7.60 (1H, t, J=7.4 Hz), 8.13 (2H, d, J=7.6 Hz), 8.40 (1H, d, J=4.4 Hz).

FAB-MS; m/z: 942 (M+H)+

### Example 98

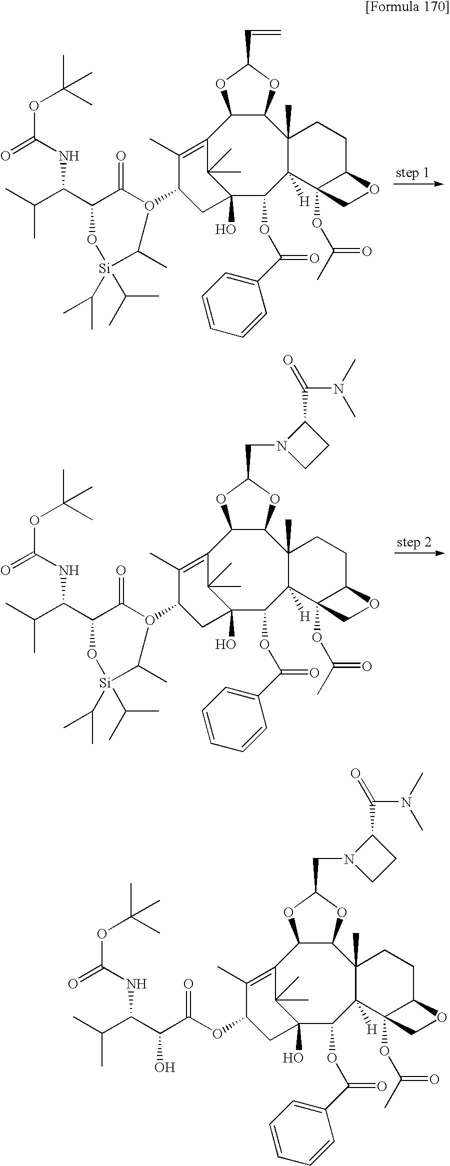

### Step 1: (1S,2S,3R,4S,5R,8R,9S,10R,13S)-4-(Acetoxy)-2-(benzoyloxy)-9,10-{(1S)-2-[(2S)-2-(dimethylcarbamoyl)azetidino]ethylidenedioxy}-5,20-epoxy-1-hydroxytax-11-en-13-yl

### (2R,3S)-3-(tert-butoxycarbonylamino)-4-methyl-2-[(triisopropylsilyl)oxy]pentanoate

The compound obtained in Example 49, Step 1 and the compound obtained in Reference Example 12, Step 2 were used as starting materials to perform the same procedure as that of Example 1 and thereby to obtain the title compound as white amorphous solid.

¹H-NMR (400 MHz, CDCl₃) δ: 1.01 (3H, d, J=6.7 Hz), 1.03 (3H, d, J=6.7 Hz), 1.07-2.33 (10H, m), 1.08-1.14 (21H, m), 1.25 (3H, s), 1.35 (9H, s), 1.49 (3H, s), 1.58 (3H, s), 1.74 (3H, s), 2.35 (3H, s), 2.83-2.92 (3H, m), 2.91 (3H, s), 2.93 (3H, s), 3.08 (1H, dd, J=15.4, 8.5 Hz), 3.55 (1H, dd, J=10.7, 5.9 Hz), 3.62 (1H, t, J=10.0 Hz), 3.95 (1H, t, J=8.5 Hz), 4.11 (1H, d, J=7.3 Hz), 4.20 (1H, d, J=8.0 Hz), 4.33 (1H, d, J=8.0 Hz), 4.70 (1H, s), 4.78 (1H, d, J=10.5 Hz), 4.92 (1H, brs), 5.01 (1H, t, J=4.6 Hz), 5.13 (1H, d, J=6.8 Hz), 5.92-6.00 (2H, m), 7.49 (2H, t, J=7.8 Hz), 7.61 (1H, t, J=7.3 Hz), 8.11-8.17 (2H, m).

### Step 2: (1S,2S,3R,4S,5R,8R,9S,10R,13S)-4-(Acetoxy)-2-(benzoyloxy)-9,10-{(1S)-2-[(2S)-2-(dimethylcarbamoyl)azetidino]ethylidenedioxy}-5,20-epoxy-1-hydroxytax-11-en-13-yl

### (2R,3S)-3-(tert-butoxycarbonylamino)-2-hydroxy-4-methylpentanoate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.02 (3H, d, J=6.7 Hz), 1.05 (3H, d, J=6.7 Hz), 1.19-2.40 (13H, m), 1.20 (3H, s), 1.40 (9H, s), 1.49 (3H, s), 1.77 (3H, s), 2.33 (3H, s), 2.84 (1H, dd, J=12.5, 5.9 Hz), 2.90-2.97 (2H, m), 2.92 (3H, s), 2.94 (3H, s), 3.09 (1H, dd, J=15.4, 8.1 Hz), 3.53-3.60 (1H, m), 3.81 (1H, t, J=9.5 Hz), 3.95 (1H, t, J=8.7 Hz), 4.05 (1H, s), 4.08-4.12 (1H, m), 4.24 (1H, d, J=8.5 Hz), 4.32 (1H, d, J=8.5 Hz), 4.49 (1H, s), 4.87 (1H, d, J=10.5 Hz), 4.93 (1H, s), 5.06 (1H, t, J=4.8 Hz), 5.19 (1H, d, J=6.3 Hz), 5.97-6.06 (2H, m), 7.48 (2H, t, J=7.7 Hz), 7.61 (1H, t, J=7.4 Hz), 8.10-8.15 (2H, m).

FAB-MS; m/z: 912 (M+H)+

### Example 99

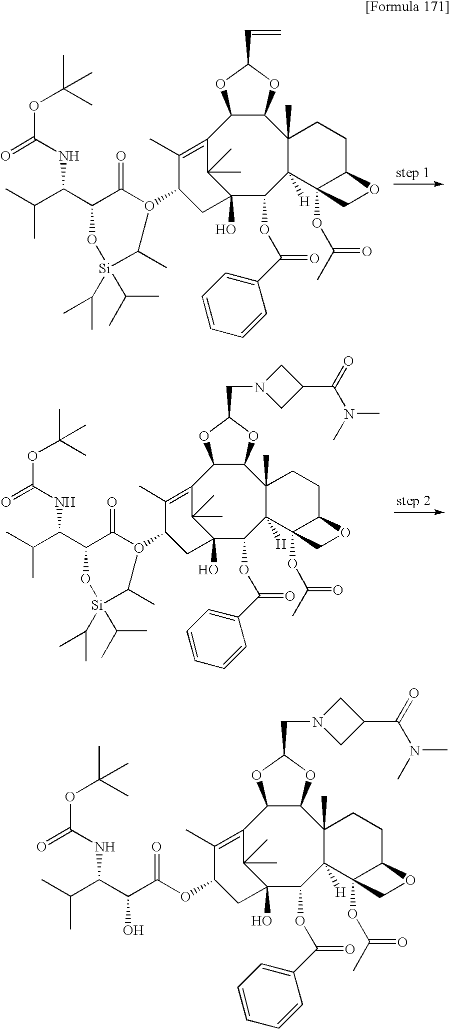

### Step 1: (1S,2S,3R,4S,5R,8R,9S,10R,13S)-4-(Acetoxy)-2-(benzoyloxy)-9,10-{(1S)-2-[3-(dimethylcarbamoyl)azetidino]ethylidenedioxy}-5,20-epoxy-1-hydroxytax-11-en-13-yl

### (2R,3S)-3-(tert-butoxycarbonylamino)-4-methyl-2-[(triisopropylsilyl)oxy]pentanoate

The compound obtained in Example 49, Step 1 and the compound obtained in Reference Example 2, Step 2 were used as starting materials to perform the same procedure as that of Example 1 and thereby to obtain the title compound as white amorphous solid.

¹H-NMR (400 MHz, CDCl₃) δ: 1.01 (3H, d, J=6.7 Hz), 1.03 (3H, d, J=6.7 Hz), 1.07-2.34 (11H, m), 1.08-1.14 (21H, m), 1.28 (3H, s), 1.35 (9H, s), 1.49 (3H, s), 1.76 (3H, s), 2.35 (3H, s), 2.75-2.91 (3H, m), 2.90 (3H, s), 2.95 (3H, s), 3.38-3.48 (2H, m), 3.54 (1H, t, J=7.1 Hz), 3.63 (1H, t, J=10.1 Hz), 3.68-3.76 (2H, m), 4.13 (1H, d, J=7.3 Hz), 4.20 (1H, d, J=8.5 Hz), 4.34 (1H, d, J=8.0 Hz), 4.71 (1H, s), 4.77 (1H, d, J=10.7 Hz), 4.84 (1H, t, J=3.9 Hz), 4.92 (1H, s), 5.16 (1H, d, J=7.3 Hz), 5.92-6.00 (2H, m), 7.49 (2H, t, J=7.8 Hz), 7.61 (1H, t, J=7.1 Hz), 8.15 (2H, d, J=7.6 Hz).

### Step 2: (1S,2S,3R,4S,5R,8R,9S,10R,13S)-4-(Acetoxy)-2-(benzoyloxy)-9,10-{(1S)-2-[3-(dimethylcarbamoyl)azetidino]ethylidenedioxy}-5,20-epoxy-1-hydroxytax-11-en-13-yl

### (2R,3S)-3-(tert-butoxycarbonylamino)-2-hydroxy-4-methylpentanoate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.02 (3H, d, J=6.7 Hz), 1.05 (3H, d, J=6.7 Hz), 1.18-2.42 (12H, m), 1.24 (3H, s), 1.38 (9H, s), 1.48 (3H, s), 1.78 (3H, s), 2.34 (3H, s), 2.76-2.90 (3H, m), 2.91 (3H, s), 2.96 (3H, s), 3.39-3.49 (2H, m), 3.50-3.59 (1H, m), 3.68-3.83 (3H, m), 4.13 (1H, d, J=7.6 Hz), 4.23 (1H, d, J=8.3 Hz), 4.32 (1H, d, J=8.5 Hz), 4.49 (1H, s), 4.84 (1H, d, J=10.0 Hz), 4.88 (1H, t, J=5.1 Hz), 4.92 (1H, s), 5.22 (1H, d, J=7.3 Hz), 5.98 (1H, d, J=4.9 Hz), 6.04 (1H, t, J=7.8 Hz), 7.48 (2H, t, J=7.8 Hz), 7.60 (1H, t, J=7.4 Hz), 8.16-8.11 (2H, m).

FAB-MS; m/z: 912 (M+H)+

### Example 100

### (1S,2S,3R,4S,5R,8R,9S,10R,13S)-4-(Acetoxy)-2-(benzoyloxy)-5,20-epoxy-9,10-[(1S)-2-(3-fluoroazetidino)ethylidenedioxy]-1-hydroxytax-11-en-13-yl

### (2R,3R)-3-(tert-butoxycarbonylamino)-4-fluoro-2-hydroxybutyrate

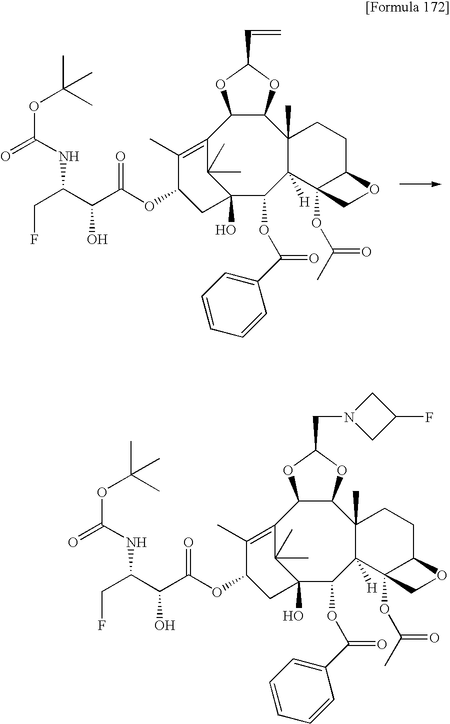

The compound obtained in Example 34, Step 2 and the compound obtained in Reference Example 24, Step 2 were used as starting materials to perform the same procedure as that of Example 1 and thereby to obtain the title compound as white amorphous solid.

¹H-NMR (400 MHz, CDCl₃) δ: 1.21-2.41 (7H, m), 1.24 (3H, s), 1.41 (9H, s), 1.49 (3H, s), 1.60 (3H, s), 1.78 (3H, s), 2.32 (3H, s), 2.82 (1H, dd, J=12.3, 5.6 Hz), 2.88-2.95 (2H, m), 3.26-3.40 (2H, m), 3.75-3.84 (1H, m), 3.79 (2H, d, J=7.6 Hz), 4.13 (1H, d, J=7.1 Hz), 4.24 (1H, d, J=8.6 Hz), 4.32 (1H, d, J=8.6 Hz), 4.37-4.55 (3H, m), 4.52 (1H, s), 4.58-4.65 (1H, m), 4.88 (1H, t, J=4.9 Hz), 4.93 (1H, s), 5.04-5.15 (1H, m), 5.19-5.25 (1H, m), 5.99 (1H, d, J=4.9 Hz), 6.06 (1H, t, J=7.6 Hz), 7.48 (2H, t, J=7.7 Hz), 7.58-7.63 (1H, m), 8.10-8.14 (2H, m).

FAB-MS; m/z: 849 (M+H)⁺.

### Example 101

### (1S,2S,3R,4S,5R,8R,9S,10R,13S)-4-(Acetoxy)-2-(benzoyloxy)-5,20-epoxy-1-hydroxy-9,10-[(1S)-2-(3-methoxyazetidino)ethylidenedioxy]tax-11-en-13-yl

### (2R,3R)-3-(tert-butoxycarbonylamino)-4-fluoro-2-hydroxybutyrate

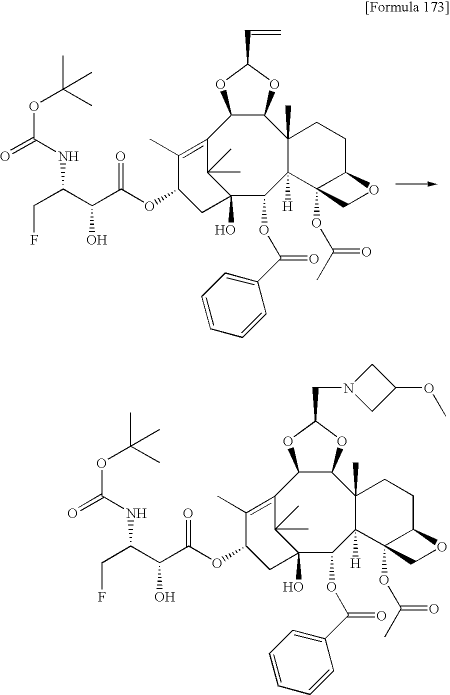

The compound obtained in Example 34, Step 2 and the compound obtained in Reference Example 1 were used as starting materials to perform the same procedure as that of Example 1 and thereby to obtain the title compound as white amorphous solid.

¹H-NMR (400 MHz, CDCl₃) δ: 1.20-2.41 (6H, m), 1.24 (3H, s), 1.41 (9H, s), 1.48 (3H, s), 1.60 (3H, s), 1.78 (3H, s), 2.33 (3H, s), 2.80 (1H, dd, J=12.5, 5.6 Hz), 2.86-2.94 (2H, m), 3.06-3.12 (2H, m), 3.27 (3H, s), 3.71-3.78 (2H, m), 4.04-4.14 (2H, m), 4.24 (1H, d, J=8.3 Hz), 4.33 (1H, d, J=8.3 Hz), 4.39-4.65 (4H, m), 4.89 (1H, t, J=4.9 Hz), 4.93 (1H, s), 5.14 (1H, d, J=8.6 Hz), 5.23 (1H, d, J=6.9 Hz), 6.00 (2H, d, J=4.9 Hz), 6.07 (2H, t, J=7.8 Hz), 7.48 (2H, t, J=7.6 Hz), 7.58-7.64 (1H, m), 8.11-8.15 (2H, m).

FAB-MS; m/z: 861 (M+H)⁺.

### Example 102

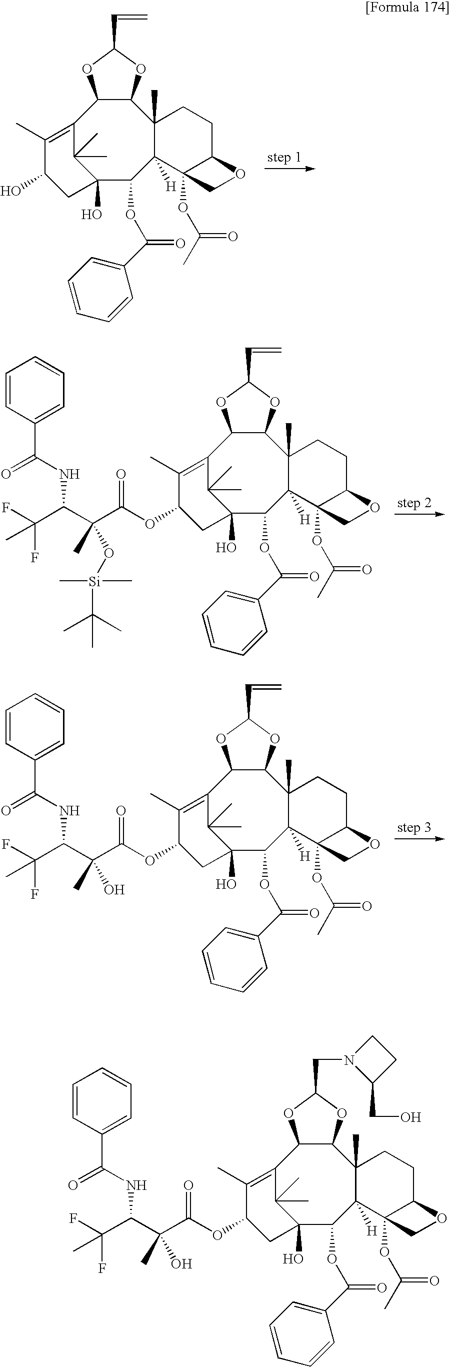

### Step 1: (1S,2S,3R,4S,5R,8R,9S,10R,13S)-4-Acetoxy-2-(benzoyloxy)-5,20-epoxy-1-hydroxy-9,10-[(1S)-2-propenylidenedioxy]tax-11-en-13-yl

### (2R,3R)-3-(benzoylamino)-2-[(tert-butyldimethylsilyl)oxy]-4,4-difluoro-2-methylpentanoate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-11-ene and the compound obtained in Reference Example 43, Step 4 were used as starting materials to perform the same procedure as that of Example 31, Step 1 and thereby to obtain the title compound as white amorphous solid. The resultant was used for the subsequent reaction as it was as a crude product.

FAB-MS; m/z: 911 (M+H)+

### Step 2: (1S,2S,3R,4S,5R,8R,9S,10R,13S)-4-Acetoxy-2-(benzoyloxy)-5,20-epoxy-1-hydroxy-9,10-[(1S)-2-propenylidenedioxy]tax-11-en-13-yl

### (2R,3R)-3-(benzoylamino)-4,4-difluoro-2-hydroxy-2-methylpentanoate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.27 (3H, s), 1.49 (3H, s), 1.61-2.24 (10H, m), 1.64 (3H, s), 1.716 (3H, s), 1.719 (3H, s), 2.48 (3H, s), 2.95 (1H, d, J=5.2 Hz), 4.14-4.17 (1H, m), 4.26 (1H, d, J=8.3 Hz), 4.33 (1H, d, J=8.3 Hz), 4.37 (1H, s), 4.94 (1H, s), 4.98-5.08 (1H, m), 5.21-5.25 (2H, m), 5.46 (1H, d, J=10.5 Hz), 5.57 (1H, d, J=17.2 Hz), 5.96-6.04 (2H, m), 6.19 (1H, t, J=8.8 Hz), 7.16 (1H, d, J=10.1 Hz), 7.44-7.63 (6H, m), 7.78-7.82 (2H, m), 8.14-8.18 (2H, m).

### Step 3: (1S,2S,3R,4S,5R,8R,9S,10R,13S)-4-Acetoxy-2-(benzoyloxy)-5,20-epoxy-1-hydroxy-9,10-{(1S)-2-[(2S)-hydroxymethylazetidino]ethylidenedioxy}tax-11-en-13-yl

### (2R,3R)-3-(benzoylamino)-4,4-difluoro-2-hydroxy-2-methylpentanoate

The compound obtained in Step 2 mentioned above and the compound obtained in Reference Example 25 were used as starting materials to perform the same procedure as that of Example 1 and thereby to obtain the title compound as white amorphous solid.

¹H-NMR (400 MHz, CDCl₃) δ: 1.25 (3H, s), 1.48 (3H, s), 1.54-2.12 (12H, m), 1.58 (3H, s), 1.70 (3H, s), 1.72 (3H, s), 2.17-2.28 (2H, m), 2.48 (3H, s), 2.80-2.94 (3H, m), 3.11 (1H, q, J=8.1 Hz), 3.40-3.53 (3H, m), 3.65 (1H, dd, J=11.4, 2.8 Hz), 4.11 (1H, d, J=7.4 Hz), 4.25 (1H, d, J=8.3 Hz), 4.30-4.35 (2H, m), 4.86 (1H, t, J=4.9 Hz), 4.93 (1H, s), 4.98-5.07 (1H, m), 5.16 (1H, d, J=7.4 Hz), 5.98 (1H, d, J=5.2 Hz), 6.17 (1H, t, J=8.8 Hz), 7.13 (1H, d, J=10.1 Hz), 7.44-7.63 (6H, m), 7.79 (2H, d, J=7.6 Hz), 8.16 (2H, d, J=7.6 Hz).

FAB-MS; m/z: 911 (M+H)+

### Example 103

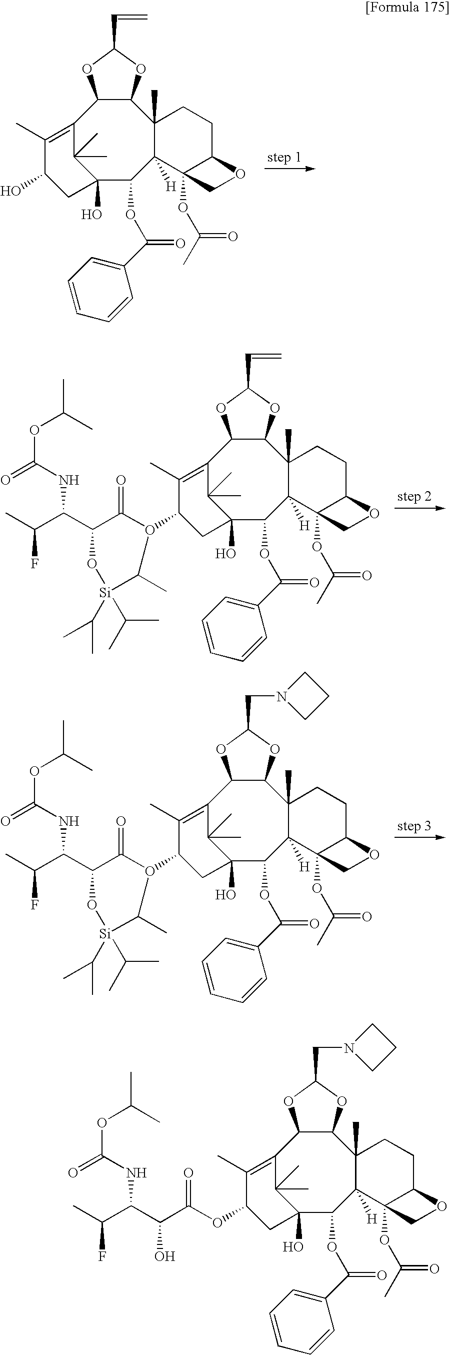

### Step 1: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3R,4S)-4-fluoro-3-(isopropoxycarbonylamino)-2-[(triisopropylsilyl)oxy]pentanoate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-1-ene and the compound obtained in Reference Example 44 were used as starting materials to perform the same procedure as that of Example 31, Step 1 and thereby to obtain the title compound as white amorphous solid.

¹H-NMR (400 MHz, CDCl₃) δ: 1.09-1.22 (27H, m), 1.30 (3H, s), 1.44 (3H, dd, J=24.2, 6.3 Hz), 1.51 (3H, s), 1.56-2.28 (7H, m), 1.64 (3H, s), 1.79 (3H, brs), 2.34 (3H, s), 2.93 (1H, d, J=4.9 Hz), 4.05-4.16 (1H, m), 4.20 (2H, d, J=7.3 Hz), 4.35 (1H, d, J=8.3 Hz), 4.54-4.84 (2H, m), 4.88-4.96 (3H, m), 5.20 (1H, d, J=6.1 Hz), 5.26 (1H, d, J=7.3 Hz), 5.45 (1H, d, J=10.3 Hz), 5.57 (1H, d, J=17.3 Hz), 5.95-6.07 (3H, m), 7.49 (2H, t, J=7.7 Hz), 7.61 (1H, t, J=7.3 Hz), 8.15 (2H, d, J=8.8 Hz).

FAB-MS; m/z: 966 (M+Na)⁺.

### Step 2: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3R,4S)-4-fluoro-3-(isopropoxycarbonylamino)-2-[(triisopropylsilyl)oxy]pentanoate

The compound obtained in Step 1 mentioned above was used as a starting material to perform the same procedure as that of Example 1 and thereby to obtain the title compound as white amorphous solid.

¹H-NMR (400 MHz, CDCl₃) δ: 1.08-1.20 (27H, m), 1.28 (3H, s), 1.43 (3H, dd, J=24.6, 6.6 Hz), 1.50 (3H, s), 1.58-2.26 (9H, m), 1.61 (3H, s), 1.76 (3H, s), 2.34 (3H, s), 2.73 (1H, dd, J=12.4, 5.9 Hz), 2.80 (1H, dd, J=12.7, 4.9 Hz), 2.91 (1H, d, J=4.9 Hz), 3.31-3.38 (4H, m), 4.04-4.11 (1H, m), 4.13 (1H, d, J=7.3 Hz), 4.19 (1H, d, J=8.3 Hz), 4.35 (1H, d, J=8.3 Hz), 4.53-4.73 (1H, m), 4.74-4.80 (1H, m), 4.83 (1H, t, J=5.4 Hz), 4.88-4.94 (3H, m), 5.17 (1H, d, J=7.6 Hz), 5.94-6.05 (2H, m), 7.49 (2H, t, J=7.7 Hz), 7.61 (1H, t, J=7.4 Hz), 8.15 (2H, d, J=8.0 Hz).

FAB-MS; m/z: 987 (M+H)⁺.

### Step 3: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3R,4S)-4-fluoro-2-hydroxy-3-(isopropoxycarbonylamino)pentanoate

The compound obtained in Step 2 mentioned above was used as a starting material to perform the same procedure as that of Example 31, Step 2 and thereby to obtain the title compound as white amorphous solid.

¹H-NMR (400 MHz, CDCl₃) δ: 1.16 (3H, d, J=6.6 Hz), 1.21 (3H, d, J=7.1 Hz), 1.24 (3H, s), 1.45 (3H, dd, J=24.9, 6.3 Hz), 1.49 (3H, s), 1.59-2.18 (10H, m), 1.61 (3H, s), 1.75 (3H, s), 2.34 (3H, s), 2.74 (1H, dd, J=12.5, 5.9 Hz), 2.82 (1H, dd, J=12.5, 4.2 Hz), 2.92 (1H, d, J=5.1 Hz), 3.30-3.40 (4H, m), 4.11 (1H, d, J=7.1 Hz), 4.24-4.36 (1H, m), 4.24 (1H, d, J=8.3 Hz), 4.33 (1H, d, J=8.5 Hz), 4.61-4.80 (1H, m), 4.70 (1H, s), 4.82-4.97 (3H, m), 5.09 (1H, d, J=10.0 Hz), 5.21 (1H, d, J=6.8 Hz), 6.01 (1H, d, J=5.1 Hz), 6.12 (1H, t, J=8.3 Hz), 7.48 (2H, t, J=7.7 Hz), 7.61 (1H, t, J=7.3 Hz), 8.13 (2H, d, J=8.8 Hz).

FAB-MS; m/z: 831 (M+H)⁺.

### Example 104

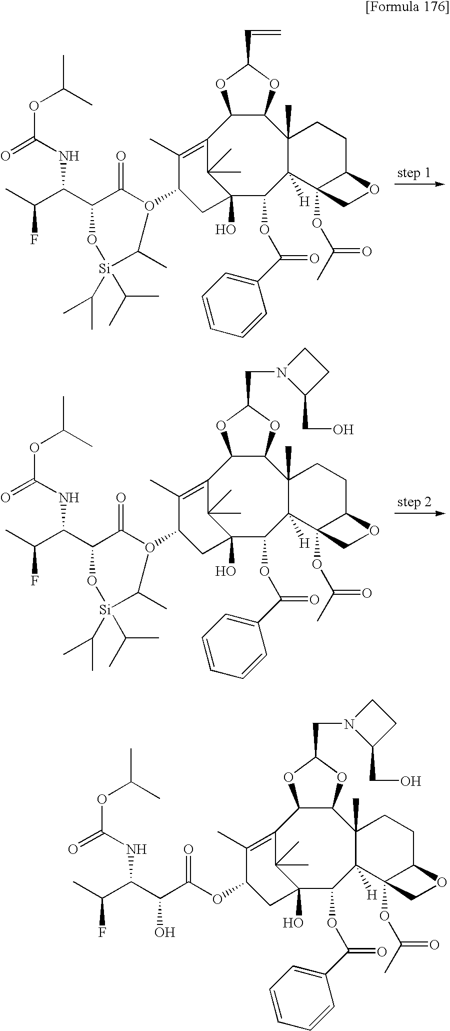

### Step 1: (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-11-en-13-yl

### (2R,3R,4S)-4-fluoro-3-(isopropoxycarbonylamino)-2-[(triisopropylsilyl)oxy]pentanoate

The compound obtained in Example 103, Step 1 and the compound obtained in Reference Example 25 were used as starting materials to perform the same procedure as that of Example 1 and thereby to obtain the title compound as white amorphous solid.

¹H-NMR (400 MHz, CDCl₃) δ: 1.07-1.21 (27H, m), 1.28 (3H, s), 1.43 (3H, dd, J=24.6, 5.9 Hz), 1.50 (3H, s), 1.55-2.28 (10H, m), 1.59 (3H, s), 1.76 (3H, s), 2.34 (3H, s), 2.80 (1H, dd, J=12.7, 5.4 Hz), 2.89 (1H, dd, J=12.9, 5.1 Hz), 2.91 (1H, d, J=5.9 Hz), 3.09 (1H, dd, J=16.1, 9.0 Hz), 3.39-3.52 (3H, m), 3.63 (1H, dd, J=11.2, 2.9 Hz), 4.04-4.12 (1H, m), 4.14 (1H, d, J=7.3 Hz), 4.19 (1H, d, J=8.8 Hz), 4.35 (1H, d, J=7.8 Hz), 4.54-4.73 (1H, m), 4.73-4.81 (1H, m), 4.84 (1H, t, J=5.1 Hz), 4.88-4.95 (3H, m), 5.19 (1H, d, J=7.3 Hz), 5.93-6.05 (2H, m), 7.50 (2H, t, J=7.6 Hz), 7.61 (1H, t, J=7.4 Hz), 8.15 (2H, d, J=7.3 Hz).

FAB-MS; m/z: 1017 (M+H)⁺.

### Step 2: (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-11-en-13-yl

### (2R,3R,4S)-4-fluoro-2-hydroxy-3-(isopropoxycarbonylamino)pentanoate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.17 (3H, d, J=7.1 Hz), 1.21 (3H, d, J=7.3 Hz), 1.23 (3H, s), 1.45 (3H, dd, J=23.9, 5.6 Hz), 1.49 (3H, s), 1.54-2.32 (11H, m), 1.58 (3H, s), 1.75 (3H, s), 2.34 (3H, s), 2.83 (1H, dd, J=12.7, 4.6 Hz), 2.86-2.94 (2H, m), 3.10 (1H, dd, J=16.1, 8.5 Hz), 3.39-3.52 (3H, m), 3.65 (1H, dd, J=11.4, 2.8 Hz), 4.12 (1H, d, J=7.1 Hz), 4.24-4.34 (1H, m), 4.24 (1H, d, J=8.5 Hz), 4.33 (1H, d, J=8.1 Hz), 4.61-4.80 (1H, m), 4.70 (1H, brs), 4.83-4.98 (3H, m), 5.11 (1H, d, J=10.3 Hz), 5.23 (1H, d, J=7.1 Hz), 5.99 (1H, d, J=5.1 Hz), 6.11 (1H, t, J=8.2 Hz), 7.48 (2H, t, J=8.3 Hz), 7.61 (1H, t, J=7.3 Hz), 8.13 (2H, d, J=8.1 Hz).

FAB-MS; m/z: 861 (M+H)⁺.

### Example 105

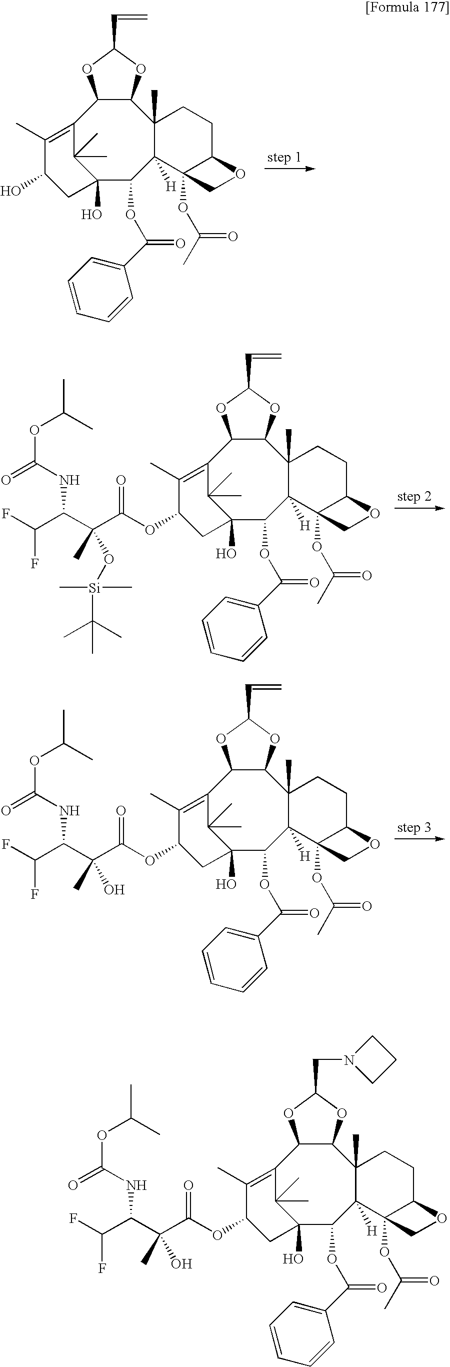

### Step 1: (1S,2S,3R,4S,5R,8R,9S,10R,13S)-4-Acetoxy-2-(benzoyloxy)-5,20-epoxy-1-hydroxy-9,10-[(1S)-2-propenylidenedioxy]tax-11-en-13-yl

### (2R,3R)-2-[(tert-butyldimethylsilyl)oxy]-4,4-difluoro-3-(isopropoxycarbonylamino)-2-m ethylbutyrate

(1S,2S,3R,4S,5R,8R,9S,10R,13S)-4-(Acetoxy)-2-(benzoyloxy)-1,13-dihydroxy-5, 20-epoxy-9,10-[(1S)-2-propenylidenedioxy]tax-11-ene and the compound obtained in Reference Example 45, Step 3 were used as starting materials to perform the same procedure as that of Example 31, Step 1 and thereby to obtain the title compound as white amorphous solid. The resultant was used for the subsequent reaction as it was as a crude product.

### Step 2: (1S,2S,3R,4S,5R,8R,9S,10R,13S)-4-Acetoxy-2-(benzoyloxy)-5,20-epoxy-1-hydroxy-9,10-[(1S)-2-propenylidenedioxy]tax-11-en-13-yl

### (2R,3R)-4,4-difluoro-2-hydroxy-3-(isopropoxycarbonylamino)-2-methylbutyrate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.18-1.30 (9H, m), 1.49 (3H, s), 1.60-2.31 (7H, m), 1.63 (3H, s), 1.65 (3H, s), 1.77 (3H, s), 2.43 (3H, s), 2.96 (1H, d, J=5.2 Hz), 4.18 (1H, d, J=7.1 Hz), 4.27 (1H, d, J=8.3 Hz), 4.33 (1H, d, J=8.6 Hz), 4.38-4.49 (1H, m), 4.66 (1H, s), 4.82-4.99 (2H, m), 5.23-5.31 (2H, m), 5.48 (2H, t, J=11.0 Hz), 5.58 (1H, d, J=17.7 Hz), 5.95-6.25 (4H, m), 7.48 (2H, t, J=7.6 Hz), 7.61 (1H, t, J=7.4 Hz), 8.15 (2H, d, J=7.4 Hz).

### Step 3: (1S,2S,3R,4S,5R,8R,9S,10R,13S)-4-Acetoxy-9,10-[(1S)-2-(azetidino)ethylidenedioxy]-2-(benzoyloxy)-5,20-epoxy-1-hydroxytax-11-en-13-yl

### (2R,3R)-4,4-difluoro-2-hydroxy-3-(isopropoxycarbonylamino)-2-methylbutyrate

The compound obtained in Step 2 mentioned above was used as a starting material to perform the same procedure as that of Example 1 and thereby to obtain the title compound as white amorphous solid.

¹H-NMR (400 MHz, CDCl₃) δ: 1.20 (3H, d, J=6.4 Hz), 1.23 (3H, d, J=6.4 Hz), 1.27 (3H, s), 1.48 (3H, s), 1.60-2.24 (9H, m), 1.62 (3H, s), 1.63 (3H, s), 1.75 (3H, s), 2.42 (3H, s), 2.75 (1H, dd, J=12.5, 5.9 Hz), 2.83 (1H, dd, J=12.5, 4.4 Hz), 2.94 (1H, d, J=5.2 Hz), 3.32-3.40 (4H, m), 4.11 (1H, d, J=6.9 Hz), 4.27 (1H, d, J=8.3 Hz), 4.33 (1H, d, J=8.3 Hz), 4.37-4.48 (1H, m), 4.64 (1H, brs), 4.85-4.94 (3H, m), 5.20 (1H, d, J=6.9 Hz), 5.47 (1H, d, J=10.5 Hz), 5.95-6.24 (3H, m), 7.49 (2H, t, J=7.8 Hz), 7.61 (1H, t, J=7.4 Hz), 8.15 (2H, d, J=7.4 Hz).

FAB-MS; m/z: 849 (M+H)⁺.

### Example 106

### (1S,2S,3R,4S,5R,8R,9S,10R,13S)-4-(Acetoxy)-2-(benzoyloxy)-5,20-epoxy-9,10-[(1S)-2-(3-fluoroazetidino)ethylidenedioxy]-1-hydroxytax-11-en-13-yl

### (2R,3S)-3-(tert-butoxycarbonylamino)-2-hydroxy-3-(2-pyrimidinyl)propionate

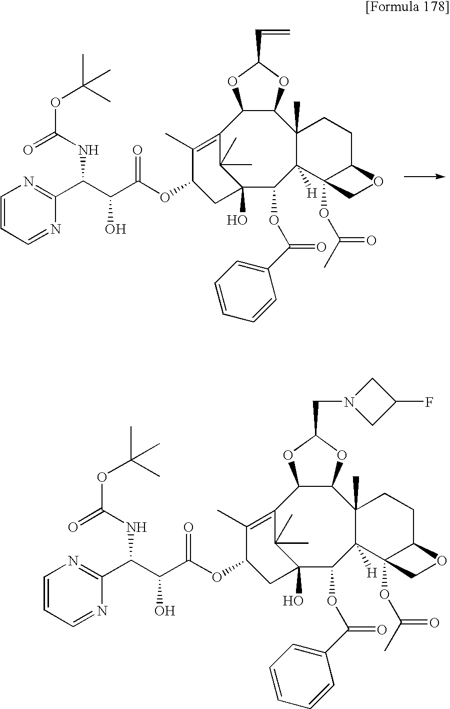

The compound obtained in Example 53, Step 2 and the compound obtained in Reference Example 24, Step 2 were used as starting materials to perform the same procedure as that of Example 1 and thereby to obtain the title compound as white amorphous solid.

¹H-NMR (400 MHz, CDCl₃) δ: 1.21-2.49 (8H, m), 1.29 (3H, s), 1.42 (9H, s), 1.50 (3H, s), 1.62 (3H, s), 1.81 (3H, s), 2.46 (3H, s), 2.83 (1H, dd, J=12.5, 5.6 Hz), 2.92 (1H, dd, J=12.4, 4.5 Hz), 2.96 (1H, d, J=5.1 Hz), 3.27-3.40 (2H, m), 3.75-3.85 (2H, m), 4.16 (1H, d, J=7.1 Hz), 4.22 (1H, d, J=8.3 Hz), 4.34 (1H, d, J=8.3 Hz), 4.88 (1H, t, J=5.0 Hz), 4.94 (1H, s), 4.99 (1H, s), 5.05-5.26 (2H, m), 5.44 (1H, dd, J=8.8, 1.5 Hz), 5.99 (1H, d, J=5.1 Hz), 6.05-6.14 (2H, m), 7.25 (2H, t, J=4.9 Hz), 7.47 (2H, t, J=7.7 Hz), 7.56-7.62 (2H, m), 8.75 (2H, d, J=4.9 Hz).

FAB-MS; m/z: 895 (M+H)⁺.

### Example 107

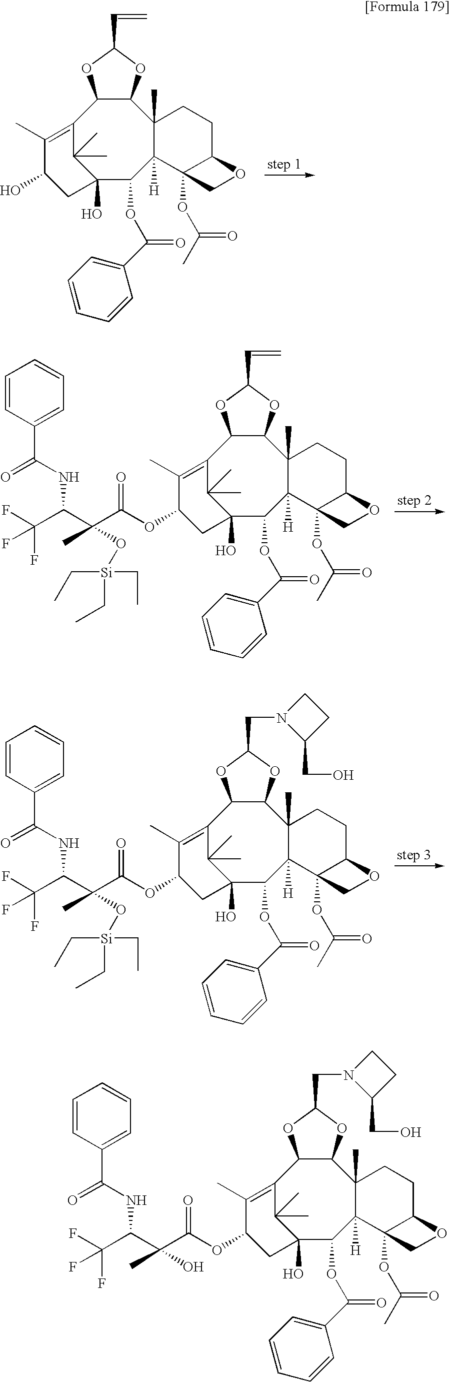

### Step 1: (1S,2S,3R,4S,5R,8R,9S,10R,13S)-4-Acetoxy-2-(benzoyloxy)-5,20-epoxy-1-hydroxy-9,10-[(1S)-2-propenylidenedioxy]tax-1′-en-13-yl

### (2R,3R)-3-(benzoylamino)-2-methyl-2-[(triethylsilyl)oxy]-4,4,4-trifluorobutyrate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-11-ene and the compound obtained in Reference Example 46, Step 3 were used as starting materials to perform the same procedure as that of Example 31, Step 1 and thereby to obtain the title compound as white amorphous solid.

¹H-NMR (400 MHz, CDCl₃) δ: 0.68-0.83 (6H, m), 1.01 (9H, t, J=7.8 Hz), 1.24 (3H, s), 1.51 (3H, s), 1.60-2.08 (9H, m), 1.63 (3H, s), 1.75 (3H, s), 2.15-2.22 (1H, m), 2.57 (3H, s), 2.87 (1H, d, J=4.9 Hz), 4.17 (1H, d, J=7.1 Hz), 4.26 (1H, d, J=8.3 Hz), 4.33 (1H, d, J=8.6 Hz), 4.94 (1H, s), 5.17-5.26 (3H, m), 5.45 (1H, d, J=10.3 Hz), 5.57 (1H, d, J=17.4 Hz), 5.95-6.10 (3H, m), 6.76 (1H, d, J=9.3 Hz), 7.40-7.45 (2H, m), 7.49-7.54 (3H, m), 7.60-7.64 (1H, m), 7.68-7.70 (2H, m), 8.17-8.21 (2H, m).

### Step 2: (1S,2S,3R,4S,5R,8R,9S,10R,13S)-4-Acetoxy-2-(benzoyloxy)-5,20-epoxy-1-hydroxy-9,10-{(1 S)-2-[(2S)-hydroxymethylazetidino]ethylidenedioxy}tax-11-en-13-yl

### (2R,3R)-3-(benzoylamino)-2-methyl-2-[(triethylsilyl)oxy]-4,4,4-trifluorobutyrate

The compound obtained in Step 1 mentioned above and the compound obtained in Reference Example 25 were used as starting materials to perform the same procedure as that of Example 1 and thereby to obtain the title compound as white amorphous solid.

¹H-NMR (400 MHz, CDCl₃) δ: 0.66-0.82 (6H, m), 1.01 (9H, t, J=8.2 Hz), 1.22 (3H, s), 1.50 (3H, s), 1.57 (3H, s), 1.60-2.03 (8H, m), 1.72 (3H, s), 1.92 (3H, s), 2.15-2.27 (2H, m), 2.56 (3H, s), 2.78-2.90 (3H, m), 3.09 (1H, q, J=8.3 Hz), 3.39-3.50 (3H, m), 3.62 (1H, dd, J=11.5, 2.9 Hz), 4.10-4.12 (1H, m), 4.25 (1H, d, J=8.3 Hz), 4.33 (1H, d, J=8.3 Hz), 4.84 (1H, t, J=4.8 Hz), 4.93 (1H, s), 5.15-5.22 (2H, m), 5.95 (1H, d, J=4.9 Hz), 6.05 (1H, t, J=9.0 Hz), 6.76 (1H, d, J=9.8 Hz), 7.42 (2H, t, J=7.8 Hz), 7.51 (3H, t, J=7.6 Hz), 7.62 (1H, t, J=7.4 Hz), 7.68 (2H, d, J=7.6 Hz), 8.18 (2H, d, J=7.6 Hz).

### Step 3: (1S,2S,3R,4S,5R,8R,9S,10R,13S)-4-Acetoxy-2-(benzoyloxy)-5,20-epoxy-1-hydroxy-9,10-{(1S)-2-[(2S)-hydroxymethylazetidino]ethylidenedioxy}tax-11-en-13-yl

### (2R,3R)-3-(benzoylamino)-2-hydroxy-2-methyl-4,4,4-trifluorobutyrate

The compound obtained in Step 2 mentioned above was used as a starting material to perform the same procedure as that of Example 31, Step 2 and thereby to obtain the title compound as white amorphous solid.

¹H-NMR (400 MHz, CDCl₃) δ: 1.22 (3H, s), 1.47 (3H, s), 1.57 (3H, s), 1.61 (3H, s), 1.62-2.28 (10H, m), 1.75 (3H, s), 2.48 (3H, s), 2.79-2.92 (3H, m), 3.09 (1H, q, J=8.1 Hz), 3.39-3.51 (3H, m), 3.63 (1H, dd, J=11.4, 3.1 Hz), 4.07 (1H, d, J=7.1 Hz), 4.26 (1H, d, J=8.5 Hz), 4.32 (1H, d, J=8.3 Hz), 4.72 (1H, brs), 4.84 (1H, t, J=5.1 Hz), 4.92 (1H, s), 5.11 (1H, d, J=7.1 Hz), 5.32-5.40 (1H, m), 5.97 (1H, d, J=5.1 Hz), 6.17 (1H, t, J=8.7 Hz), 7.11 (1H, d, J=10.0 Hz), 7.43-7.56 (5H, m), 7.61 (1H, t, J=7.3 Hz), 7.78 (2H, d, J=7.6 Hz), 8.15 (2H, d, J=7.1 Hz).

FAB-MS; m/z: 915 (M+H)⁺.

### Example 108

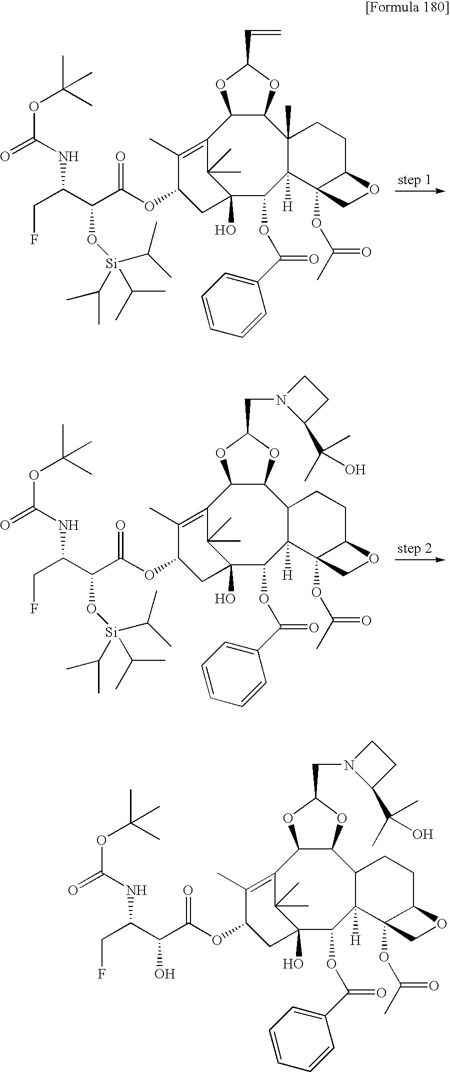

### Step 1: (1S,2S,3R,4S,5R,8R,9S,10R,13S)-4-(Acetoxy)-2-(benzoyloxy)-5,20-epoxy-9,10-{(1S)-2-[(2S)-2-(1-hydroxy-1-methylethyl)azetidino]ethylidenedioxy}-1-hydroxytax-11-en-13-yl

### (2R,3R)-3-(tert-butoxycarbonylamino)-4-fluoro-2-[(triisopropylsilyl)oxy]butyrate

The compound obtained in Example 34, Step 1 and the compound obtained in Reference Example 37 were used as starting materials to perform the same procedure as that of Example 1 and thereby to obtain the title compound as white amorphous solid.

¹H-NMR (400 MHz, CDCl₃) δ: 1.02 (3H, s), 1.11-1.21 (21H, m), 1.27 (3H, s), 1.37 (9H, s), 1.50 (3H, s), 1.59-2.02 (13H, m), 1.77 (3H, s), 2.17-2.27 (3H, m), 2.33 (3H, s), 2.80 (1H, dd, J=12.8, 5.4 Hz), 2.93 (1H, d, J=5.4 Hz), 3.01-3.06 (2H, m), 3.21 (1H, t, J=8.0 Hz), 3.48-3.53 (1H, m), 4.17 (2H, dd, J=12.3, 7.8 Hz), 4.32-4.48 (4H, m), 4.78-4.80 (2H, m), 4.86 (1H, t, J=5.0 Hz), 4.91 (1H, s), 5.19 (1H, d, J=7.4 Hz), 5.95 (1H, d, J=5.2 Hz), 6.02 (1H, t, J=9.0 Hz), 7.49 (2H, t, J=7.6 Hz), 7.61 (1H, t, J=7.4 Hz), 8.15 (2H, d, J=7.1 Hz).

### Step 2: (1S,2S,3R,4S,5R,8R,9S,10R,13S)-4-(Acetoxy)-2-(benzoyloxy)-5,20-epoxy-9,10-{(1S)-2-[(2S)-2-(1-hydroxy-1-methylethyl)azetidino]ethylidenedioxy}-1-hydroxytax-11-en-13-yl

### (2R,3R)-3-(tert-butoxycarbonylamino)-4-fluoro-2-hydroxybutyrate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.03 (3H, s), 1.15 (3H, s), 1.24 (3H, s), 1.41 (9H, s), 1.49 (3H, s), 1.60-2.03 (11H, m), 1.79 (3H, m), 2.18-2.41 (2H, m), 2.33 (3H, s), 2.83 (1H, dd, J=12.8, 5.2 Hz), 2.93 (1H, d, J=5.1 Hz), 3.00-3.07 (2H, m), 3.22 (1H, t, J=7.9 Hz), 3.49-3.53 (1H, m), 4.15 (1H, d, J=7.1 Hz), 4.24 (1H, d, J=8.5 Hz), 4.33 (1H, d, J=8.3 Hz), 4.41-4.53 (5H, m), 4.90 (1H, t, J=4.8 Hz), 4.94 (1H, s), 5.10 (1H, d, J=8.3 Hz), 5.25 (1H, d, J=7.3 Hz), 5.99 (1H, d, J=4.9 Hz), 6.05-6.11 (1H, m), 7.48 (2H, t, J=7.7 Hz), 7.61 (1H, t, J=7.4 Hz), 8.13 (2H, d, J=7.3 Hz).

FAB-MS; m/z: 889 (M+H)⁺.

### Example 109

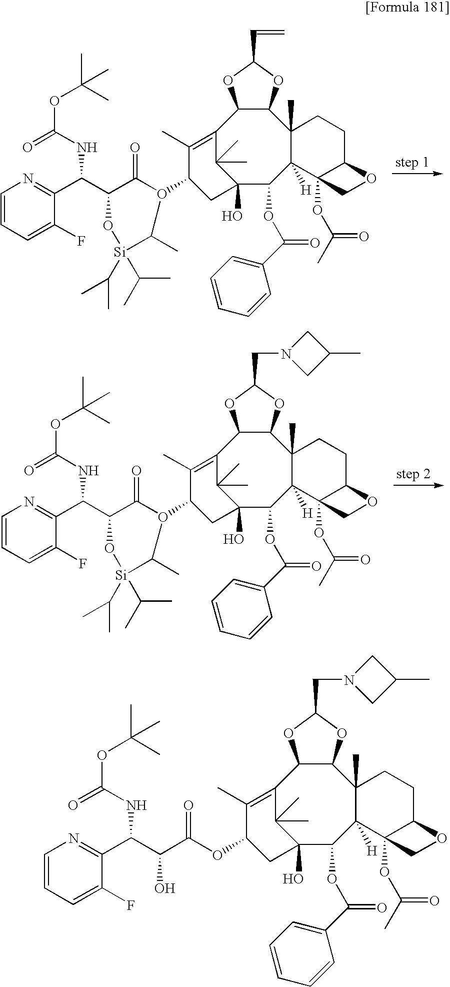

### Step 1: (1S,2S,3R,4S,5R,8R,9S,10R,13S)-4-(Acetoxy)-2-(benzoyloxy)-5,20-epoxy-1-hydroxy-9,10-[(1S)-2-(3-methylazetidino)ethylidenedioxy]tax-11-en-13-yl

### (2R,3S)-3-(tert-butoxycarbonylamino)-3-(3-fluoro-2-pyridyl)-2-[(triisopropylsilyl)oxy]propionate

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-3-(3-fluoro-2-pyridyl)-2-[(triisopropylsilyl)oxy]propionate (Bioorg. Med. Chem. Lett., 2004, 14, 3209) and the compound obtained in Reference Example 47 were used as starting materials to perform the same procedure as that of Example 1 and thereby to obtain the title compound as white amorphous solid.

¹H-NMR (400 MHz, CDCl₃) δ: 0.80-1.00 (21H, m), 1.16 (3H, d, J=6.6 Hz), 1.21-2.11 (4H, m), 1.35 (3H, s), 1.38 (9H, s), 1.53 (3H, s), 1.63 (3H, s), 1.79 (3H, s), 2.27-2.42 (2H, m), 2.48 (3H, s), 2.58-2.88 (6H, m), 2.97 (1H, d, J=5.6 Hz), 3.61 (2H, dd, J=12.8, 5.7 Hz), 4.15 (1H, d, J=7.3 Hz), 4.22 (1H, d, J=8.3 Hz), 4.35 (1H, d, J=8.3 Hz), 4.84 (1H, t, J=5.0 Hz), 4.92-4.99 (2H, m), 5.18 (1H, d, J=7.1 Hz), 5.60 (1H, d, J=9.5 Hz), 5.97 (1H, d, J=5.1 Hz), 6.03-6.15 (2H, m), 7.23-7.30 (1H, m), 7.37-7.42 (1H, m), 7.47 (2H, t, J=7.6 Hz), 7.55-7.63 (1H, m), 8.13-8.20 (2H, m), 8.40 (1H, d, J=4.6 Hz).

ESI-MS; m/z: 1065 (M+H)⁺.

### Step 2: (1S,2S,3R,4S,5R,8R,9S,10R,13S)-4-(Acetoxy)-2-(benzoyloxy)-5,20-epoxy-1-hydroxy-9,10-[(1S)-2-(3-methylazetidino)ethylidenedioxy]tax-11-en-13-yl

### (2R,3S)-3-(tert-butoxycarbonylamino)-3-(3-fluoro-2-pyridyl)-2-hydroxypropionate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.00-2.25 (7H, m), 1.18 (3H, d, J=6.8 Hz), 1.28 (3H, s), 1.39 (9H, d, J=12.9 Hz), 1.50 (3H, s), 1.62 (3H, s), 1.78 (3H, s), 2.34 (3H, s), 2.45 (1H, t, J=12.2 Hz), 2.59-3.03 (6H, m), 3.58-3.75 (2H, m), 4.13 (1H, d, J=7.1 Hz), 4.23 (1H, d, J=8.5 Hz), 4.33 (1H, d, J=8.5 Hz), 4.67 (1H, d, J=2.4 Hz), 4.91 (2H, s), 5.22 (1H, d, J=7.3 Hz), 5.65 (1H, d, J=8.8 Hz), 5.99 (1H, d, J=5.1 Hz), 6.08 (1H, t, J=8.4 Hz), 6.20 (1H, d, J=8.8 Hz), 7.25-7.35 (1H, m), 7.42-7.49 (3H, m), 7.60 (1H, t, J=7.4 Hz), 8.09-8.18 (2H, m), 8.40 (1H, d, J=4.6 Hz).

ESI-MS; m/z: 908 (M+H)⁺.

### Example 110

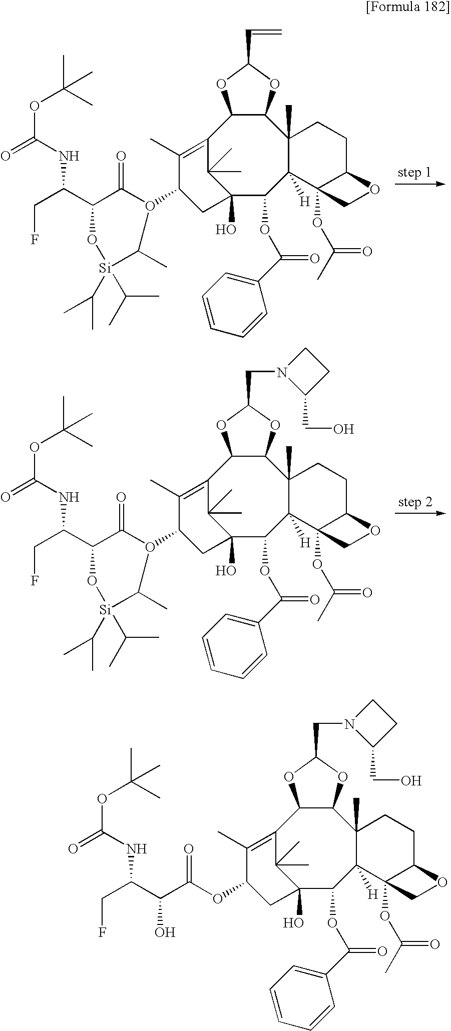

### Step 1: (1S,2S,3R,4S,5R,8R,9S,10R,13S)-4-(Acetoxy)-2-(benzoyloxy)-5,20-epoxy-1-hydroxy-9,10-{(1S)-2-[(2R)-2-hydroxymethylazetidino]ethylidenedioxy}tax-11-en-13-yl

### (2R,3R)-3-(tert-butoxycarbonylamino)-4-fluoro-2-[(triisopropylsilyl)oxy]butyrate

The compound obtained in Example 34, Step 1 and the compound obtained in Reference Example 26 were used as starting materials to perform the same procedure as that of Example 1 and thereby to obtain the title compound as white amorphous solid.

¹H-NMR (400 MHz, CDCl₃) δ: 1.04-1.20 (21H, m), 1.27 (3H, s), 1.37 (9H, s), 1.50 (3H, s), 1.60 (3H, s), 1.77 (3H, s), 1.87-2.06 (7H, m), 2.20-2.27 (3H, m), 2.33 (3H, s), 2.77 (1H, dd, J=12.8, 5.5 Hz), 2.91 (2H, dd, J=13.0, 4.4 Hz), 3.07 (1H, q, J=8.3 Hz), 3.41-3.51 (3H, m), 3.64 (1H, dd, J=11.3, 2.7 Hz), 4.10-4.20 (2H, m), 4.32-4.48 (4H, m), 4.79-4.80 (2H, m), 4.84 (1H, t, J=5.0 Hz), 4.91 (1H, s), 5.18 (1H, d, J=7.4 Hz), 5.96 (1H, d, J=5.2 Hz), 6.02 (1H, t, J=9.0 Hz), 7.50 (2H, t, J=7.6 Hz), 7.61 (1H, t, J=7.5 Hz), 8.15 (2H, d, J=7.4 Hz).

### Step 2: (1S,2S,3R,4S,5R,8R,9S,10R,13S)-4-(Acetoxy)-2-(benzoyloxy)-5,20-epoxy-1-hydroxy-9,10-{(1 S)-2-[(2R)-2-hydroxymethylazetidino]ethylidenedioxy}tax-11-en-13-yl

### (2R,3R)-3-(tert-butoxycarbonylamino)-4-fluoro-2-hydroxybutyrate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.18-2.07 (9H, m), 1.24 (3H, s), 1.42 (9H, s), 1.48 (3H, s), 1.61 (3H, s), 1.79 (3H, s), 2.20-2.41 (2H, m), 2.33 (3H, s), 2.79 (1H, dd, J=12.7, 5.6 Hz), 2.88-2.95 (2H, m), 3.08 (1H, q, J=8.2 Hz), 3.41-3.52 (3H, m), 3.65 (1H, dd, J=11.4, 2.8 Hz), 4.13 (1H, d, J=6.9 Hz), 4.24 (1H, d, J=8.3 Hz), 4.33 (1H, d, J=8.5 Hz), 4.38-4.63 (4H, m), 4.89 (1H, t, J=4.9 Hz), 4.94 (1H, s), 5.11 (1H, d, J=8.8 Hz), 5.23 (1H, d, J=6.9 Hz), 6.01 (1H, d, J=5.1 Hz), 6.07 (1H, t, J=8.2 Hz), 7.49 (2H, t, J=7.6 Hz), 7.60-7.64 (1H, m), 8.13 (2H, d, J=8.1 Hz).

FAB-MS; m/z: 861 (M+H)⁺.

### Example 111

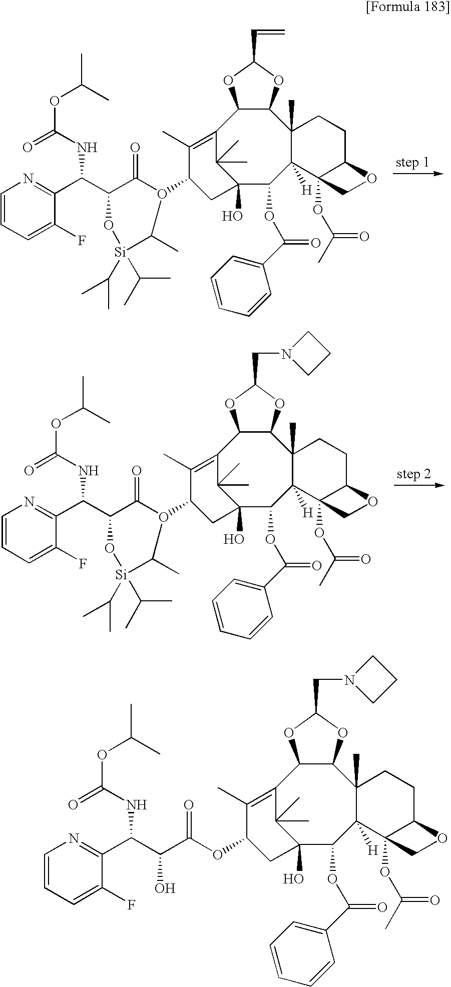

### Step 1: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3S)-3-(3-fluoro-2-pyridyl)-3-(isopropoxycarbonylamino)-2-[(triisopropylsilyl)oxy]propionate

The compound obtained in Example 82, Step 1 was used as a starting material to perform the same procedure as that of Example 1 and thereby to obtain the title compound as white amorphous solid.

¹H-NMR (400 MHz, CDCl₃) δ: 0.90-0.94 (21H, m), 1.17-1.21 (6H, m), 1.35 (3H, s), 1.52 (3H, s), 1.63 (3H, s), 1.78 (3H, s), 1.91-2.14 (7H, m), 2.33 (2H, d, J=9.3 Hz), 2.47 (3H, s), 2.71-2.83 (2H, m), 2.96 (1H, d, J=5.1 Hz), 3.33-3.37 (4H, m), 4.15 (1H, d, J=7.3 Hz), 4.22 (1H, d, J=8.3 Hz), 4.36 (1H, d, J=8.1 Hz), 4.79-4.84 (2H, m), 4.95-4.98 (2H, m), 5.17 (1H, d, J=7.3 Hz), 5.62 (1H, d, J=9.5 Hz), 5.98 (1H, d, J=5.1 Hz), 6.07-6.14 (2H, m), 7.24-7.28 (1H, m), 7.40 (1H, t, J=8.8 Hz), 7.47 (2H, t, J=7.7 Hz), 7.59 (1H, t, J=7.5 Hz), 8.16 (2H, d, J=7.3 Hz), 8.38 (1H, d, J=4.6 Hz).

### Step 2: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3S)-3-(3-fluoro-2-pyridyl)-2-hydroxy-3-(isopropoxycarbonylamino)propionate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.19-1.24 (6H, m), 1.28 (3H, s), 1.49 (3H, s), 1.62 (3H, s), 1.76 (3H, s), 1.81-1.93 (5H, m), 2.03-2.16 (4H, m), 2.33 (3H, s), 2.38-2.44 (1H, m), 2.72-2.85 (2H, m), 2.93 (1H, d, J=4.9 Hz), 3.32-3.40 (4H, m), 4.12 (1H, d, J=7.1 Hz), 4.23 (1H, d, J=8.3 Hz), 4.32 (1H, d, J=7.8 Hz), 4.70 (1H, d, J=2.9 Hz), 4.85-4.91 (3H, m), 5.21 (1H, d, J=7.1 Hz), 5.71 (1H, d, J=8.8 Hz), 6.00 (1H, d, J=5.1 Hz), 6.11 (1H, t, J=8.4 Hz), 6.27 (1H, d, J=8.5 Hz), 7.28-7.32 (1H, m), 7.42-7.49 (3H, m), 7.60 (1H, t, J=7.4 Hz), 8.13 (2H, dd, J=8.3, 1.2 Hz), 8.40 (1H, d, J=4.6 Hz).

FAB-MS; m/z: 880 (M+H)⁺.

### Example 112

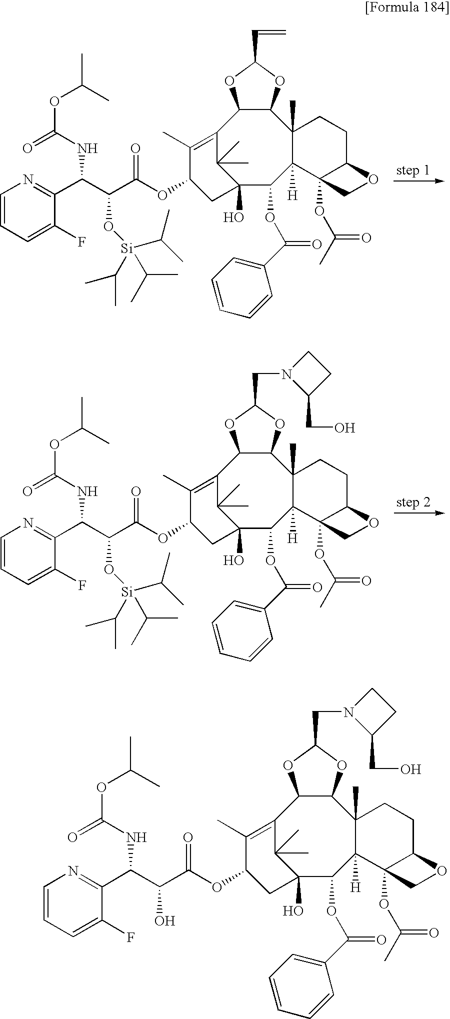

### Step 1: (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-11-en-13-yl

### (2R,3S)-3-(3-fluoro-2-pyridyl)-3-(isopropoxycarbonylamino)-2-[(triisopropylsilyl)oxy]propionate

The compound obtained in Example 82, Step 1 and the compound obtained in Reference Example 25 were used as starting materials to perform the same procedure as that of Example 1 and thereby to obtain the title compound as white amorphous solid.

¹H-NMR (400 MHz, CDCl₃) δ: 0.90-0.94 (21H, m), 1.18-1.21 (6H, m), 1.35 (3H, s), 1.52 (3H, s), 1.61 (3H, s), 1.79 (3H, s), 1.92-2.05 (7H, m), 2.21-2.34 (3H, m), 2.48 (3H, s), 2.79-2.97 (3H, m), 3.08-3.13 (1H, m), 3.41-3.51 (3H, m), 3.64 (1H, dd, J=11.6, 2.6 Hz), 4.20 (2H, dd, J=18.1, 7.8 Hz), 4.36 (1H, d, J=8.3 Hz), 4.79-4.85 (2H, m), 4.95-4.97 (2H, m), 5.20 (1H, d, J=7.6 Hz), 5.62 (1H, d, J=9.6 Hz), 5.96 (1H, d, J=4.9 Hz), 6.07-6.15 (2H, m), 7.25-7.29 (1H, m), 7.38-7.49 (3H, m), 7.59 (1H, t, J=6.9 Hz), 8.16 (2H, d, J=8.1 Hz), 8.39 (1H, d, J=4.4 Hz).

### Step 2: (1S,2S,3R,4S,5R,8R,9S,10R,13S)-4-(Acetoxy)-2-(benzoyloxy)-5,20-epoxy-1-hydroxy-9,1 o-{(1S)-2-[(2S)-2-hydroxymethylazetidino]ethylidenedioxy}tax-11-en-13-yl

### (2R,3S)-3-(3-fluoro-2-pyridyl)-2-hydroxy-3-(isopropoxycarbonylamino)propionate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.19-1.23 (6H, m), 1.28 (3H, s), 1.49 (3H, s), 1.59 (3H, s), 1.76 (3H, s), 1.81-2.09 (9H, m), 2.20-2.42 (2H, m) 2.33 (3H, s), 2.80-2.93 (3H, m), 3.10 (1H, q, J=8.2 Hz), 3.41-3.51 (3H, m), 3.66 (1H, dd, J=11.5, 3.2 Hz), 4.13 (1H, d, J=7.3 Hz), 4.23 (1H, d, J=8.3 Hz), 4.32 (1H, d, J=7.6 Hz), 4.70 (1H, s), 4.85-4.91 (3H, m), 5.23 (1H, d, J=7.1 Hz), 5.70 (1H, d, J=8.5 Hz), 5.98 (1H, d, J=5.1 Hz), 6.11 (1H, t, J=8.5 Hz), 6.27 (1H, d, J=8.1 Hz), 7.28-7.33 (1H, m), 7.43-7.49 (3H, m), 7.60 (1H, t, J=7.3 Hz), 8.13 (2H, d, J=7.1 Hz), 8.40 (1H, d, J=4.6 Hz).

FAB-MS; m/z: 910 (M+H)⁺.

### Example 113

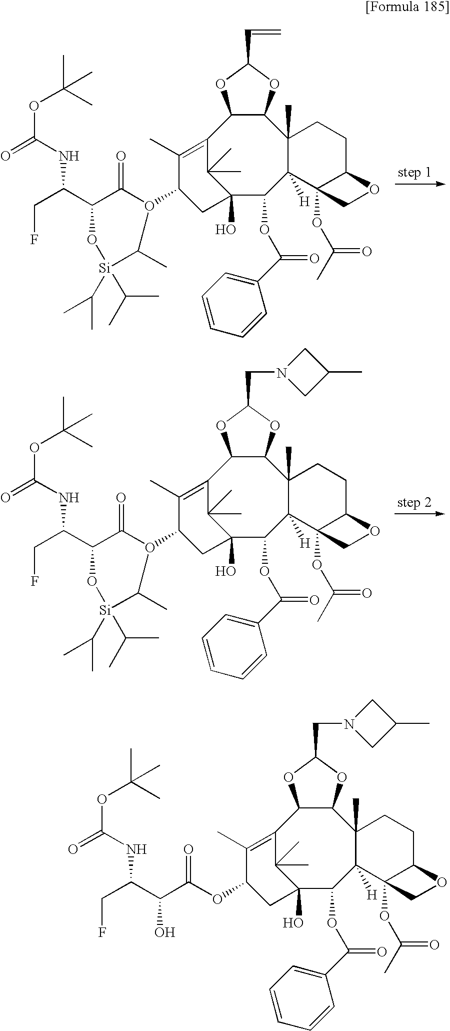

### Step 1: (1S,2S,3R,4S,5R,8R,9S,10R,13S)-4-(Acetoxy)-2-(benzoyloxy)-5,20-epoxy-1-hydroxy-9,10-[(1S)-2-(3-methylazetidino)ethylidenedioxy]tax-11-en-13-yl

### (2R,3R)-3-(tert-butoxycarbonylamino)-4-fluoro-2-[(triisopropylsilyl)oxy]butyrate

The compound obtained in Example 34, Step 1 and the compound obtained in Reference Example 47 were used as starting materials to perform the same procedure as that of Example 1 and thereby to obtain the title compound as white amorphous solid.

ESI-MS; m/z: 1001 (M+H)⁺.

### Step 2: (1S,2S,3R,4S,5R,8R,9S,10R,13S)-4-(Acetoxy)-2-(benzoyloxy)-5,20-epoxy-1-hydroxy-9,10-[(1S)-2-(3-methylazetidino)ethylidenedioxy]tax-11-en-13-yl

### (2R,3R)-3-(tert-butoxycarbonylamino)-4-fluoro-2-hydroxybutyrate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.17 (3H, d, J=6.8 Hz), 1.24 (3H, s), 1.28-2.44 (7H, m), 1.41 (9H, s), 1.49 (3H, s), 1.61 (3H, s), 1.79 (3H, s), 2.33 (3H, s), 2.63-2.93 (7H, m), 3.57-3.71 (2H, m), 4.12 (1H, d, J=7.1 Hz), 4.25 (1H, d, J=8.3 Hz), 4.33 (1H, d, J=8.3 Hz), 4.37-4.69 (4H, m), 4.86-4.98 (2H, m), 5.11 (1H, d, J=9.0 Hz), 5.23 (1H, d, J=7.3 Hz), 6.01 (1H, d, J=4.9 Hz), 6.03-6.12 (1H, m), 7.44-7.54 (2H, m), 7.61 (1H, t, J=7.4 Hz), 8.13 (2H, d, J=7.3 Hz).

ESI-MS; m/z: 845 (M+H)⁺.

### Example 114

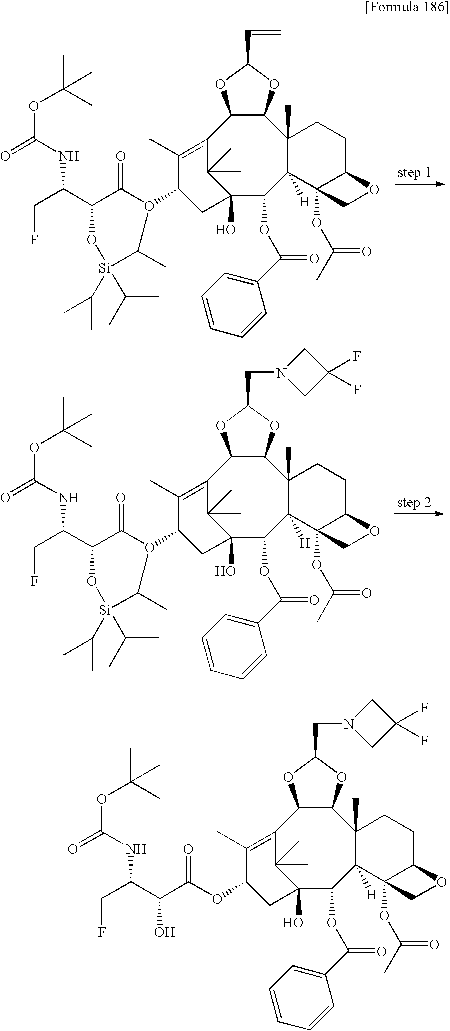

### Step 1: (1S,2S,3R,4S,5R,8R,9S,10R,13S)-4-(Acetoxy)-2-(benzoyloxy)-9,10-[(1S)-2-(3,3-difluoroazetidino)ethylidenedioxy]-5,20-epoxy-1-hydroxytax-11-en-13-yl

### (2R,3R)-3-(tert-butoxycarbonylamino)-4-fluoro-2-[(triisopropylsilyl)oxy]butyrate

The compound obtained in Example 34, Step 1 and the compound obtained in Reference Example 48, Step 2 were used as starting materials to perform the same procedure as that of Example 1 and thereby to obtain the title compound as white amorphous solid.

ESI-MS; m/z: 1023 (M+H)⁺.

### Step 2: (1S,2S,3R,4S,5R,8R,9S,10R,13S)-4-(Acetoxy)-2-(benzoyloxy)-9,10-[(1S)-2-(3,3-difluoroazetidino)ethylidenedioxy]-5,20-epoxy-1-hydroxytax-11-en-13-yl

### (2R,3R)-3-(tert-butoxycarbonylamino)-4-fluoro-2-hydroxybutyrate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.25 (3H, s), 1.33-2.45 (7H, m), 1.41 (9H, s), 1.49 (3H, s), 1.60 (3H, s), 1.80 (3H, s), 2.33 (3H, s), 2.88 (1H, dd, J=12.2, 5.6 Hz), 2.93 (1H, d, J=5.1 Hz), 2.99 (1H, dd, J=12.2, 4.0 Hz), 3.74 (4H, td, J=12.0, 2.0 Hz), 4.14 (1H, d, J=6.8 Hz), 4.24 (1H, d, J=8.5 Hz), 4.29-4.67 (5H, m), 4.33 (1H, d, J=8.5 Hz), 4.87-4.98 (2H, m), 5.10 (1H, d, J=8.8 Hz), 5.24 (1H, d, J=6.8 Hz), 6.00 (1H, d, J=4.9 Hz), 6.07 (1H, t, J=8.3 Hz), 7.49 (2H, t, J=7.8 Hz), 7.62 (1H, t, J=7.8 Hz), 8.13 (2H, d, J=7.8 Hz).

ESI-MS; m/z: 867 (M+H)⁺.

### Example 115

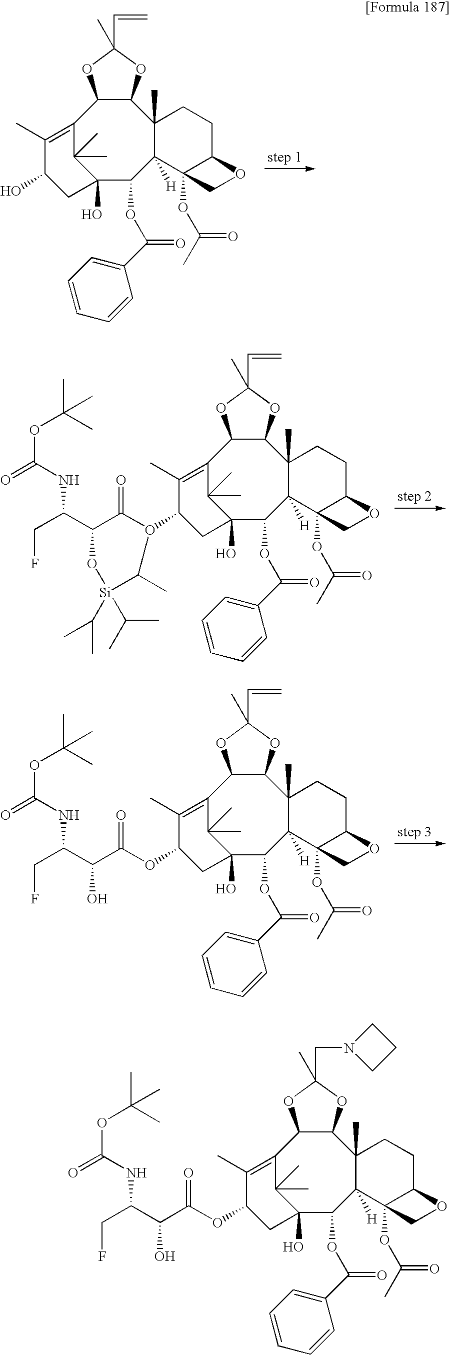

### Step 1: (1S,2S,3R,4S,5R,8R,9S,10R,13S)-4-(Acetoxy)-2-(benzoyloxy)-5,20-epoxy-1-hydroxy-9,10-[(1-methyl)-2-propenylidenedioxy]tax-11-en-13-yl

### (2R,3R)-3-(tert-butoxycarbonylamino)-4-fluoro-2-[(triisopropylsilyl)oxy]butyrate

The compound obtained in Example 81, Step 1 and the compound obtained in Reference Example 18, Step 4 were used as starting materials to perform the same procedure as that of Example 31, Step 1 and thereby to obtain the title compound as white amorphous solid.

¹H-NMR (400 MHz, CDCl₃) δ: 1.08-1.19 (21H, m), 1.08-2.34 (6H, m), 1.30 (3H, s), 1.38 (9H, s), 1.43 (3H, s), 1.47 (3H, s), 1.65 (3H, s), 1.81 (3H, s), 2.33 (3H, s), 2.92 (1H, d, J=4.9 Hz), 4.18 (1H, d, J=8.6 Hz), 4.24 (1H, d, J=7.1 Hz), 4.31-4.52 (5H, m), 4.76-4.81 (2H, m), 4.92 (1H, s), 5.20 (1H, dd, J=10.9, 1.1 Hz), 5.44 (1H, dd, J=17.5, 1.1 Hz), 5.60 (1H, d, J=7.1 Hz), 5.95 (1H, d, J=5.1 Hz), 6.01-6.11 (2H, m), 7.49, (2H, t, J=7.6 Hz), 7.58-7.63 (1H, m), 8.13-8.17 (2H, m).

### Step 2: (1S,2S,3R,4S,5R,8R,9S,10R,13S)-4-(Acetoxy)-2-(benzoyloxy)-5,20-epoxy-1-hydroxy-9,10-[(1-methyl)-2-propenylidenedioxy]tax-11-en-13-yl

### (2R,3R)-3-(tert-butoxycarbonylamino)-4-fluoro-2-hydroxybutyrate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.24-2.41 (7H, m), 1.26 (3H, s), 1.42 (9H, s), 1.50 (3H, s), 1.59 (3H, s), 1.65 (3H, s), 1.82 (3H, s), 2.33 (3H, s), 2.92 (1H, d, J=4.9 Hz), 4.19-4.26 (2H, m), 4.32 (1H, d, J=8.3 Hz), 4.38-4.66 (5H, m), 4.94 (1H, s), 5.07-5.14 (1H, m), 5.21 (1H, d, J=11.0 Hz), 5.44 (1H, d, J=17.4 Hz), 5.64 (1H, d, J=7.1 Hz), 6.00 (1H, d, J=5.1 Hz), 6.03-6.12 (2H, m), 7.48 (2H, t, J=7.8 Hz), 7.57-7.63 (1H, m), 8.11-8.15 (2H, m).

### Step 3: (1S,2S,3R,4S,5R,8R,9S,10R,13S)-4-(Acetoxy)-2-(benzoyloxy)-5,20-epoxy-1-hydroxy-9,10-[(1-methyl)-2-(azetidino)ethylidenedioxy]tax-11-en-13-yl

### (2R,3R)-3-(tert-butoxycarbonylamino)-4-fluoro-2-hydroxybutyrate

The compound obtained in Step 2 mentioned above was used as a starting material to perform the same procedure as that of Example 1 and thereby to obtain the title compound as white amorphous solid.

¹H-NMR (400 MHz, CDCl₃) δ: 1.22-2.39 (9H, m), 1.24 (3H, s), 1.41 (9H, s), 1.44 (3H, s), 1.54 (3H, s), 1.61 (3H, s), 1.80 (3H, s), 2.33 (3H, s), 2.73 (1H, d, J=12.7 Hz), 2.77 (1H, d, J=12.7 Hz), 2.94 (1H, d, J=4.9 Hz), 3.33-3.39 (4H, m), 4.17 (1H, d, J=7.4 Hz), 4.26 (1H, d, J=8.6 Hz), 4.33 (1H, d, J=8.6 Hz), 4.40-4.65 (5H, m), 4.94 (1H, s), 5.09 (1H, d, J=9.1 Hz), 5.60 (1H, d, J=7.4 Hz), 5.99 (1H, d, J=4.9 Hz), 6.08 (1H, t, J=7.8 Hz), 7.48 (2H, t, J=7.7 Hz), 7.61 (1H, t, J=7.5 Hz), 8.13 (2H, d, J=7.6 Hz).

FAB-MS; m/z: 845 (M+H)⁺.

### Example 116

### (1S,2S,3R,4S,5R,8R,9S,10R,13S)-4-(Acetoxy)-2-(benzoyloxy)-5,20-epoxy-1-hydroxy-9,10-{1-methyl-2-[(2S)-2-hydroxymethylazetidino]ethylidenedioxy}tax-11-en-13-yl

### (2R,3R)-3-(tert-butoxycarbonylamino)-4-fluoro-2-hydroxybutyrate

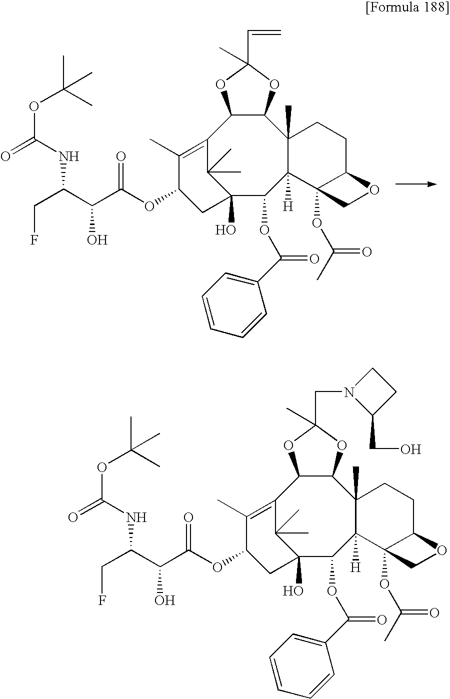

The compound obtained in Example 115, Step 2 and the compound obtained in Reference Example 25 were used as starting materials to perform the same procedure as that of Example 1 and thereby to obtain the title compound as white amorphous solid.

¹H-NMR (400 MHz, CDCl₃) δ: 1.22-2.39 (10H, m), 1.23 (3H, s), 1.41 (9H, s), 1.45 (3H, s), 1.54 (3H, s), 1.58 (3H, s), 1.80 (3H, s), 2.33 (3H, s), 2.77 (1H, d, J=12.7 Hz), 2.83 (1H, d, J=12.7 Hz), 2.94 (1H, d, J=5.1 Hz), 3.11 (1H, q, J=7.8 Hz), 3.41 (1H, d, J=11.5 Hz), 3.45-3.53 (2H, m), 3.62 (1H, dd, J=11.5, 2.9 Hz), 4.19 (1H, d, J=7.6 Hz), 4.27 (1H, s), 4.33 (1H, d, J=8.3 Hz), 4.39-4.65 (5H, m), 4.94 (1H, s), 5.08 (1H, d, J=9.1 Hz), 5.61 (1H, d, J=7.6 Hz), 5.97 (1H, d, J=5.1 Hz), 6.07 (1H, t, J=7.6 Hz), 7.48 (2H, t, J=7.6 Hz), 7.61 (1H, t, J=7.4 Hz), 8.13 (2H, d, J=7.6 Hz).

FAB-MS; m/z: 875 (M+H)⁺.

### Example 117

### (1S,2S,3R,4S,5R,8R,9S,10R,13S)-4-(Acetoxy)-2-(benzoyloxy)-5,20-epoxy-1-hydroxy-9,10-{(1S)-2-[(2R)-2-methylazetidino]ethylidenedioxy}tax-11-en-13-yl

### (2R,3R)-3-(tert-butoxycarbonylamino)-4-fluoro-2-hydroxybutyrate

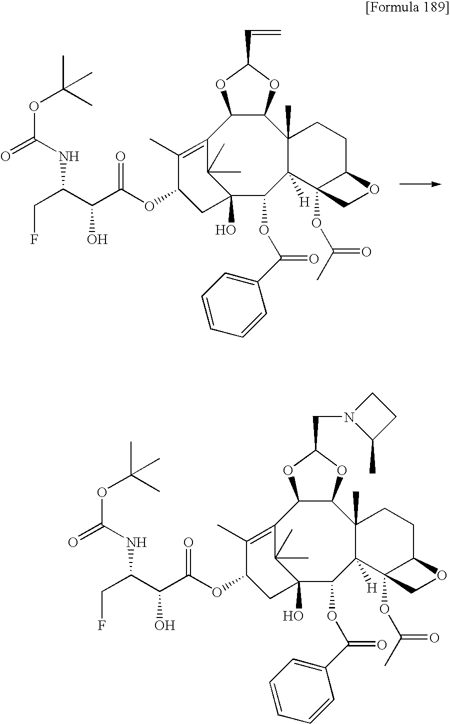

The compound obtained in Example 34, Step 2 and the compound obtained in Reference Example 49 were used as starting materials to perform the same procedure as that of Example 1 and thereby to obtain the title compound as white amorphous solid.

¹H-NMR (400 MHz, CDCl₃) δ: 1.15-2.41 (8H, m), 1.23 (3H, d, J=6.1 Hz), 1.24 (3H, s), 1.41 (9H, s), 1.49 (3H, s), 1.61 (3H, s), 1.79 (3H, s), 2.33 (3H, s), 2.74 (1H, dd, J=12.6, 4.3 Hz), 2.87 (1H, dd, J=12.7, 5.4 Hz), 2.91-3.01 (2H, m), 3.25-3.33 (1H, m), 3.50-3.54 (1H, m), 4.12 (1H, d, J=7.1 Hz), 4.25 (1H, d, J=8.6 Hz), 4.33 (1H, d, J=8.6 Hz), 4.39-4.65 (4H, m), 4.91 (2H, t, J=5.4 Hz), 5.12 (1H, d, J=8.6 Hz), 5.24 (1H, d, J=7.1 Hz), 6.01 (2H, d, J=4.9 Hz), 6.07 (2H, t, J=7.6 Hz), 7.48 (2H, t, J=7.7 Hz), 7.61 (1H, t, J=7.4 Hz), 8.13 (2H, d, J=7.6 Hz).

FAB-MS; m/z: 845 (M+H)⁺.

### Example 118

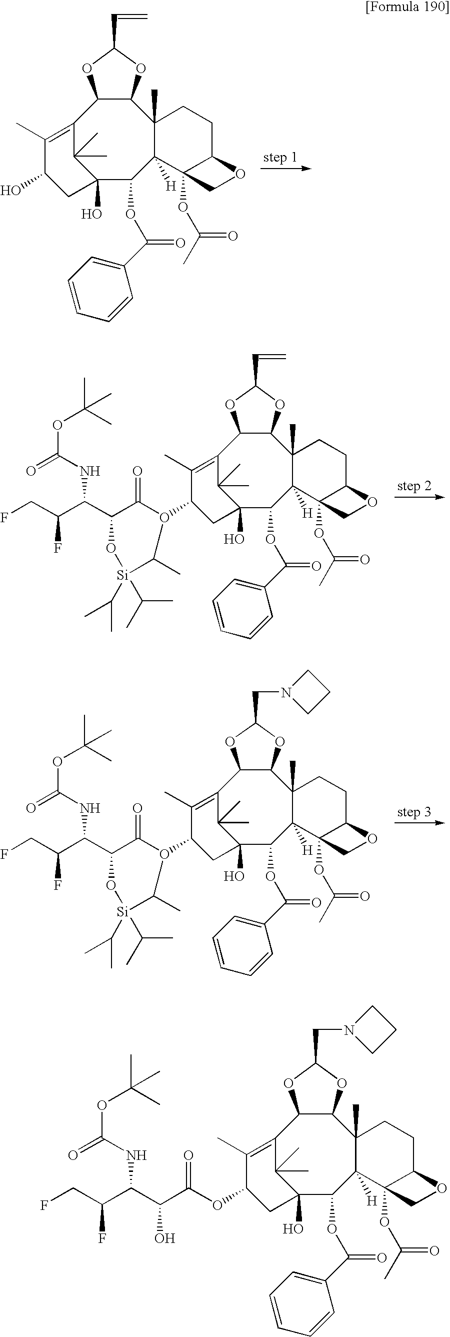

### Step 1: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3R,4R)-3-(tert-butoxycarbonylamino)-4,5-difluoro-2-[(triisopropylsilyl)oxy]pentanoate

(1S,2S,3R,4S,5R,8R,9S,10R,13S)-4-(Acetoxy)-2-(benzoyloxy)-1,13-dihydroxy-5, 20-epoxy-9,10-[(1S)-2-propenylidenedioxy]tax-11-ene and the compound obtained in Reference Example 50, Step 5 were used as starting materials to perform the same procedure as that of Example 31, Step 1 and thereby to obtain the title compound as white amorphous solid.

¹H-NMR (400 MHz, CDCl₃) δ: 1.05-1.22 (21H, m), 1.27 (3H, s), 1.32-2.44 (7H, m), 1.37 (9H, s), 1.51 (3H, s), 1.58 (3H, s), 1.79 (3H, s), 2.32 (3H, s), 2.92 (1H, d, J=4.9 Hz), 4.16-4.22 (2H, m), 4.31-4.41 (2H, m), 4.51-4.99 (6H, m), 5.20 (1H, d, J=6.3 Hz), 5.26 (1H, d, J=8.4 Hz), 5.45 (1H, d, J=10.3 Hz), 5.57 (1H, d, J=17.3 Hz), 5.94-6.06 (3H, m), 7.44-7.54 (2H, m), 7.61 (1H, t, J=7.4 Hz), 8.15 (2H, d, J=7.4 Hz).

### Step 2: (1S,2S,3R,4S,5R,8R,9S,10R,13S)-4-(Acetoxy)-2-(benzoyloxy)-5,20-epoxy-1-hydroxy-9,10-[(1S)-2-(azetidino)ethylidenedioxy]tax-11-en-13-yl

### (2R,3R,4R)-3-(tert-butoxycarbonylamino)-4,5-difluoro-2-[(triisopropylsilyl)oxy]pentanoate

The compound obtained in Step 1 mentioned above was used as a starting material to perform the same procedure as that of Example 1 and thereby to obtain the title compound as white amorphous solid.

ESI-MS; m/z: 1019 (M+H)⁺.

### Step 3: (1S,2S,3R,4S,5R,8R,9S,10R,13S)-4-(Acetoxy)-2-(benzoyloxy)-5,20-epoxy-1-hydroxy-9,10-[(1S)-2-(azetidino)ethylidenedioxy]tax-11-en-13-yl

### (2R,3R,4R)-3-(tert-butoxycarbonylamino)-4,5-difluoro-2-hydroxypentanoate

The compound obtained in Step 2 mentioned above was used as a starting material to perform the same procedure as that of Example 31, Step 2 and thereby to obtain the title compound as white amorphous solid.

¹H-NMR (400 MHz, CDCl₃) δ: 1.24 (3H, s), 1.24-2.41 (10H, m), 1.40 (9H, s), 1.49 (3H, s), 1.61 (3H, s), 1.77 (3H, s), 2.34 (3H, s), 2.72-2.95 (3H, m), 3.42 (4H, brs), 4.12 (1H, d, J=7.1 Hz), 4.24 (1H, d, J=8.3 Hz), 4.34 (1H, d, J=8.3 Hz), 4.45-4.94 (7H, m), 5.05 (1H, d, J=9.8 Hz), 5.23 (1H, d, J=6.8 Hz), 6.01 (1H, d, J=5.4 Hz), 6.04-6.16 (1H, m), 7.43-7.53 (2H, m), 7.62 (1H, t, J=7.4 Hz), 8.13 (2H, d, J=7.4 Hz).

ESI-MS; m/z: 863 (M+H)⁺.

### Example 119

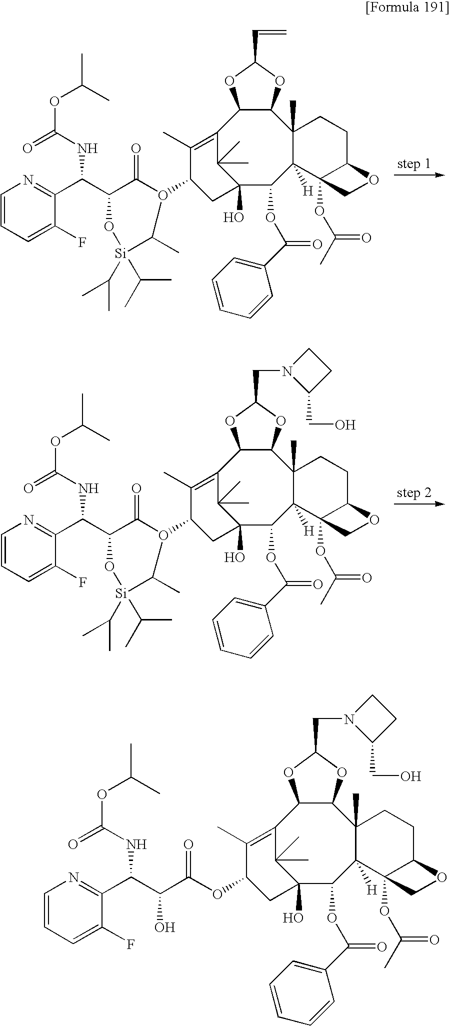

### Step 1: (1S,2S,3R,4S,5R,8R,9S,10R,13S)-4-(Acetoxy)-2-(benzoyloxy)-5,20-epoxy-1-hydroxy-9,10-{(1S)-2-[(2R)-2-hydroxymethylazetidino]ethylidenedioxy}tax-11-en-13-yl

### (2R,3S)-3-(3-fluoro-2-pyridyl)-3-(isopropoxycarbonylamino)-2-[(triisopropylsilyl)oxy]propionate

The compound obtained in Example 82, Step 1 and the compound obtained in Reference Example 26 were used as starting materials to perform the same procedure as that of Example 1 and thereby to obtain the title compound as white amorphous solid.

¹H-NMR (400 MHz, CDCl₃) δ: 0.90-0.94 (21H, m), 1.14-1.22 (8H, m), 1.35 (3H, s), 1.51 (3H, s), 1.62 (3H, s), 1.79 (3H, s), 1.91-2.00 (5H, m), 2.21-2.34 (3H, m), 2.47 (3H, s), 2.78 (1H, dd, J=12.9, 5.5 Hz), 2.89-2.97 (2H, m), 3.08 (1H, q, J=8.3 Hz), 3.42-3.51 (3H, m), 3.65 (1H, dd, J=11.4, 2.8 Hz), 4.19 (2H, dd, J=15.2, 7.8 Hz), 4.36 (1H, d, J=8.1 Hz), 4.79-4.86 (2H, m), 4.95-4.98 (2H, m), 5.18 (1H, d, J=7.1 Hz), 5.62 (1H, d, J=9.8 Hz), 5.97 (1H, d, J=4.9 Hz), 6.09-6.15 (2H, m), 7.25-7.29 (1H, m), 7.38-7.49 (3H, m), 7.59 (1H, t, J=7.4 Hz), 8.16 (2H, d, J=7.4 Hz), 8.38 (1H, d, J=4.7 Hz).

### Step 2: (1S,2S,3R,4S,5R,8R,9S,10R,13S)-4-(Acetoxy)-2-(benzoyloxy)-5,20-epoxy-1-hydroxy-9,10-{(1S)-2-[(2R)-2-hydroxymethylazetidino]ethylidenedioxy}tax-11-en-13-yl

### (2R,3S)-3-(3-fluoro-2-pyridyl)-2-hydroxy-3-(isopropoxycarbonylamino)propionate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.14-1.26 (8H, m), 1.28 (3H, s), 1.49 (3H, s), 1.61 (3H, s), 1.76 (3H, s), 1.81-2.09 (7H, m), 2.22-2.44 (2H, m), 2.33 (3H, s), 2.79 (1H, dd, J=12.7, 5.6 Hz), 2.91-2.95 (2H, m), 3.08 (1H, q, J=8.3 Hz), 3.42-3.50 (3H, m), 3.65 (1H, dd, J=11.5, 2.9 Hz), 4.09-4.14 (1H, m), 4.23 (1H, d, J=8.6 Hz), 4.32 (1H, d, J=8.3 Hz), 4.70 (1H, d, J=2.9 Hz), 4.83-4.91 (3H, m), 5.22 (1H, d, J=7.1 Hz), 5.70 (1H, d, J=8.8 Hz), 5.99 (1H, d, J=4.9 Hz), 6.11 (1H, t, J=8.3 Hz), 6.27 (1H, d, J=8.6 Hz), 7.28-7.32 (1H, m), 7.44-7.49 (3H, m), 7.60 (1H, t, J=7.4 Hz), 8.13 (2H, d, J=7.4 Hz), 8.40 (1H, d, J=4.7 Hz).

FAB-MS; m/z: 910 (M+H)⁺.

### Example 120

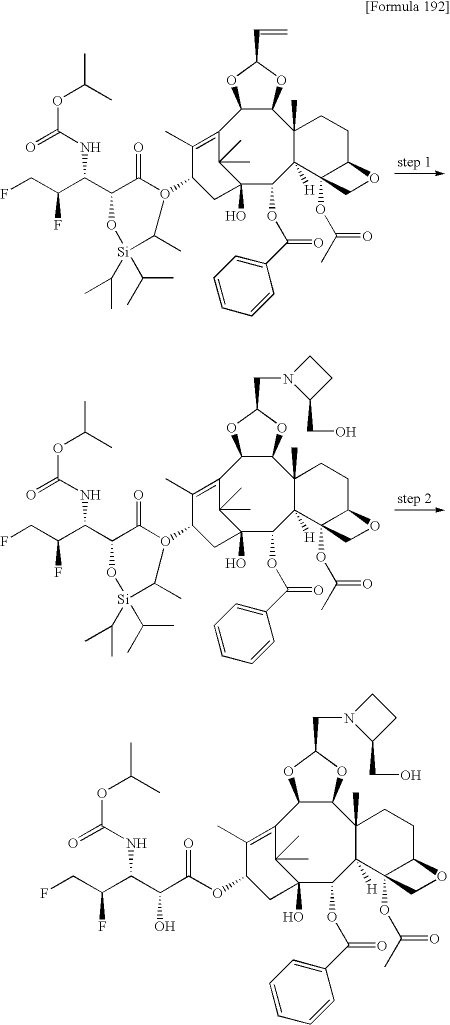

### Step 1: (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-11-en-13-yl

### (2R,3R,4R)-3-(tert-butoxycarbonylamino)-4,5-difluoro-2-[(triisopropylsilyl)oxy]pentanoate

The compound obtained in Example 118, Step 1 and the compound obtained in Reference Example 25 were used as starting materials to perform the same procedure as that of Example 1 and thereby to obtain the title compound as white amorphous solid.

ESI-MS; m/z: 1049 (M+H)⁺.

### Step 2: (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-11-en-13-yl

### (2R,3R,4R)-3-(tert-butoxycarbonylamino)-4,5-difluoro-2-hydroxypentanoate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.23 (3H, s), 1.29-2.41 (11H, m), 1.40 (9H, s), 1.49 (3H, s), 1.59 (3H, s), 1.77 (3H, s), 2.34 (3H, s), 2.80-2.98 (3H, m), 3.14 (1H, q, J=8.2 Hz), 3.39-3.59 (3H, m), 3.62-3.72 (1H, m), 4.13 (1H, d, J=7.1 Hz), 4.24 (1H, d, J=8.5 Hz), 4.34 (1H, d, J=8.5 Hz), 4.45-4.94 (7H, m), 5.07 (1H, d, J=10.0 Hz), 5.24 (1H, d, J=7.3 Hz), 5.99 (1H, d, J=5.1 Hz), 6.03-6.16 (1H, m), 7.43-7.53 (2H, m), 7.62 (1H, t, J=7.3 Hz), 8.13 (2H, d, J=7.3 Hz).

ESI-MS; m/z: 893 (M+H)⁺.

### Example 121

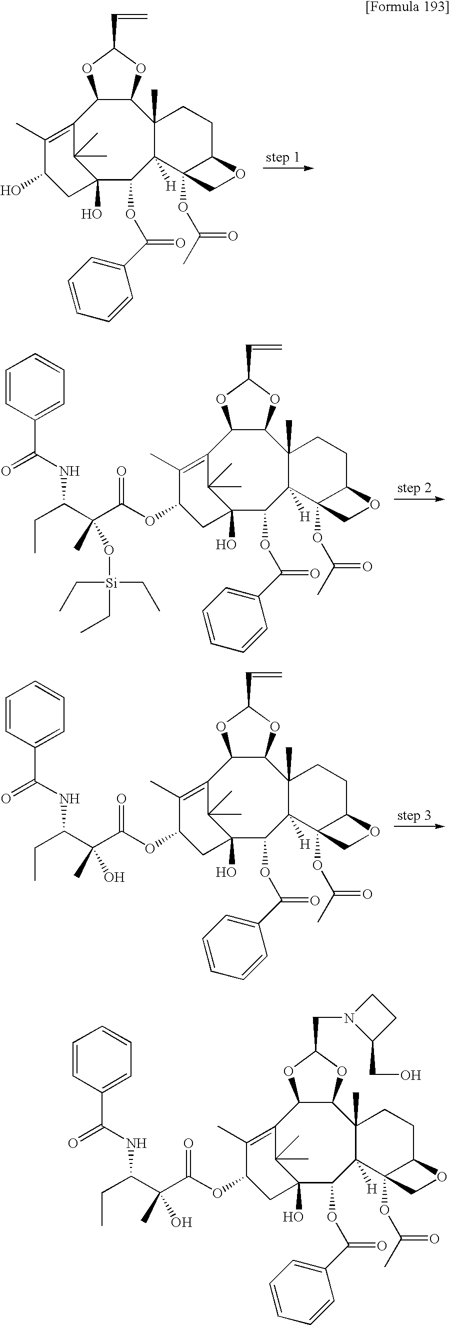

### Step 1: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3S)-3-(benzoylamino)-2-methyl-2-[(triethylsilyl)oxy]pentanoate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-11-ene and the compound obtained in Reference Example 54, Step 6 were used as starting materials to perform the same procedure as that of Example 31, Step 1 and thereby to obtain the title compound as white amorphous solid, which was used as it was for the following reaction.

### Step 2: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3S)-3-(benzoylamino)-2-hydroxy-2-methylpentanoate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.01 (3H, t, J=7.1 Hz), 1.26 (3H, s), 1.49 (3H, s), 1.52-2.24 (9H, m), 1.61 (3H, s), 1.63 (3H, s), 1.68 (3H, s), 2.50 (3H, s), 2.92 (1H, d, J=5.1 Hz), 4.14 (1H, d, J=7.6 Hz), 4.26 (1H, d, J=9.0 Hz), 4.30-4.36 (2H, m), 4.54 (1H, dt, J=3.7, 10.2 Hz), 4.95 (1H, s), 5.19-5.24 (2H, m), 5.46 (1H, d, J=10.5 Hz), 5.57 (1H, d, J=17.6 Hz), 5.94-6.05 (2H, m), 6.19 (1H, t, J=8.9 Hz), 6.56 (1H, d, J=10.5 Hz), 7.39-7.64 (6H, m), 7.75 (2H, d, J=8.5 Hz), 8.16 (2H, d, J=9.0 Hz).

ESI-MS; m/z: 802 (M+H)⁺.

### Step 3: (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-11-en-13-yl

### (2R,3S)-3-(benzoylamino)-2-hydroxy-2-methylpentanoate

The compound obtained in Step 2 mentioned above and the compound obtained in Reference Example 52 were used as starting materials to perform the same procedure as that of Example 1 and thereby to obtain the title compound as white amorphous solid.

¹H-NMR (400 MHz, CDCl₃) δ: 1.01 (3H, t, J=7.4 Hz), 1.24 (3H, s), 1.45-2.29 (12H, m), 1.48 (3H, s), 1.58 (3H, s), 1.61 (3H, s), 1.66 (3H, s), 2.50 (3H, s), 2.81 (1H, dd, J=12.7, 5.1 Hz), 2.85-2.92 (2H, m), 3.05-3.13 (1H, m), 3.38-3.52 (3H, m), 3.64 (1H, dd, J=11.5, 2.9 Hz), 4.09 (1H, d, J=7.1 Hz), 4.23-4.36 (3H, m), 4.54 (1H, td, J=10.7, 2.8 Hz), 4.85 (1H, t, J=4.9 Hz), 4.94 (1H, s), 5.15 (1H, d, J=7.1 Hz), 5.98 (1H, d, J=5.4 Hz), 6.17 (1H, t, J=8.4 Hz), 6.54 (1H, d, J=10.3 Hz), 7.39-7.64 (6H, m), 7.74 (2H, d, J=8.5 Hz), 8.16 (2H, d, J=8.5 Hz).

ESI-MS; m/z: 875 (M+H)⁺.

### Example 122

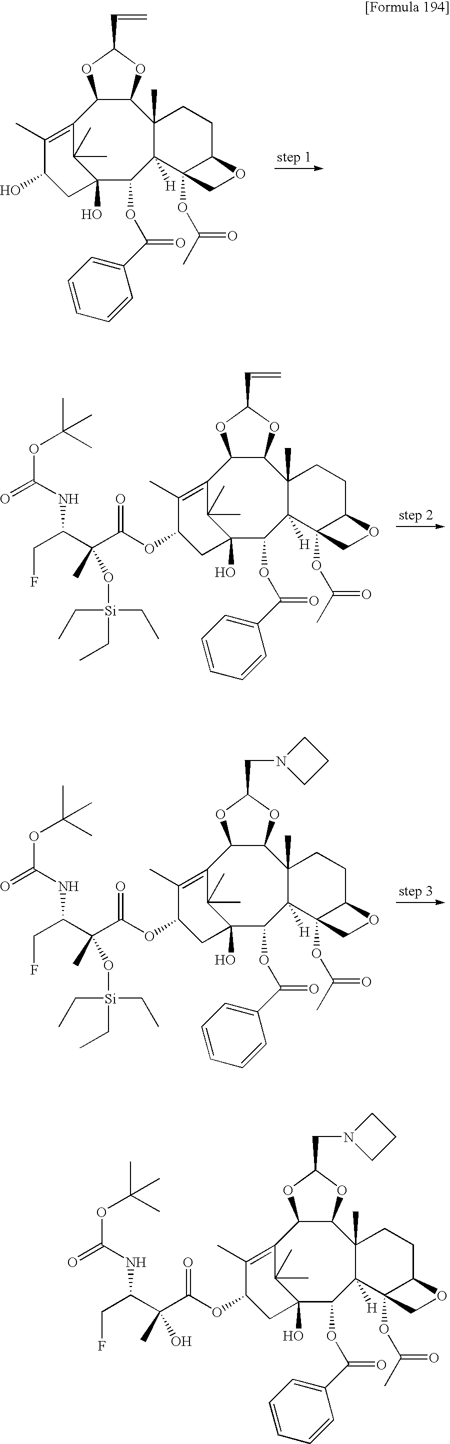

### Step 1: (1S,2S,3R,4S,5R,8R,9S,10R,13S)-4-(Acetoxy)-2-(benzoyloxy)-5,20-epoxy-1-hydroxy-9,10-[(1S)-(2-propenylidenedioxy)]tax-11-en-13-yl

### (2R,3R)-3-(tert-butoxycarbonylamino)-4-fluoro-2-methyl-2-[(triethylsilyl)oxy]butyrate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-11-ene and the compound obtained in Reference Example 55, Step 7 were used as starting materials to perform the same procedure as that of Example 31, Step 1 and thereby to obtain the title compound as white amorphous solid.

¹H-NMR (400 MHz, CDCl₃) δ: 0.61-0.79 (6H, m), 0.98 (9H, t, J=8.0 Hz), 1.34 (3H, s), 1.35 (9H, s), 1.40-2.25 (7H, m), 1.51 (3H, s), 1.65 (3H, s), 1.74 (3H, s), 1.76 (3H, s), 2.49 (3H, s), 2.89 (1H, d, J=4.9 Hz), 4.18 (1H, d, J=7.2 Hz), 4.24 (1H, d, J=8.3 Hz), 4.30-4.45 (3H, m), 4.57-4.74 (1H, m), 4.83 (1H, d, J=9.3 Hz), 4.91 (1H, s), 5.21 (1H, d, J=6.4 Hz), 5.26 (1H, d, J=7.4 Hz), 5.46 (1H, d, J=10.3 Hz), 5.58 (1H, d, J=17.4 Hz), 5.95-6.04 (2H, m), 6.14 (1H, t, J=9.3 Hz), 7.49 (2H, t, J=7.4 Hz), 7.60 (1H, t, J=6.9 Hz), 8.16 (2H, d, J=8.3 Hz).

### Step 2: (1S,2S,3R,4S,5R,8R,9S,10R,13S)-4-Acetoxy-9,10-[(1S)-2-(azetidino)ethylidenedioxy]-2-(benzoyloxy)-5,20-epoxy-1-hydroxytax-11-en-13-yl

### (2R,3R)-3-(tert-butoxycarbonylamino)-4-fluoro-2-methyl-2-[(triethylsilyl)oxy]butyrate

The compound obtained in Step 1 mentioned above was used as a starting material to perform the same procedure as that of Example 1 and thereby to obtain the title compound as white amorphous solid.

¹H-NMR (400 MHz, CDCl₃) δ: 0.61-0.79 (6H, m), 0.98 (9H, t, J=7.8 Hz), 1.33 (3H, s), 1.34-2.24 (10H, m), 1.35 (9H, s), 1.51 (3H, s), 1.61 (3H, s), 1.73 (3H, s), 1.74 (3H, s), 2.49 (3H, s), 2.82-2.95 (2H, m), 3.49-3.54 (3H, m), 4.22-4.36 (3H, m), 4.40-4.45 (1H, m), 4.57-4.62 (1H, m), 4.68-4.78 (2H, m), 4.84 (1H, d, J=10.0 Hz), 4.89-4.95 (2H, m), 5.17 (1H, d, J=7.1 Hz), 5.94 (1H, d, J=5.1 Hz), 6.13 (1H, t, J=8.8 Hz), 7.49 (2H, t, J=7.7 Hz), 7.61 (1H, t, J=7.8 Hz), 8.16 (2H, d, J=7.4 Hz).

### Step 3: (1S,2S,3R,4S,5R,8R,9S,10R,13S)-4-Acetoxy-9,10-[(1S)-2-(azetidino)ethylidenedioxy]-2-(benzoyloxy)-5,20-epoxy-1-hydroxytax-11-en-13-yl

### (2R,3R)-3-(tert-butoxycarbonylamino)-4-fluoro-2-hydroxy-2-methylbutyrate

The compound obtained in Step 2 mentioned above was used as a starting material to perform the same procedure as that of Example 31, Step 2 and thereby to obtain the title compound as white amorphous solid.

¹H-NMR (400 MHz, CDCl₃) δ: 1.28 (3H, s), 1.41 (9H, s), 1.49 (3H, s), 1.60-2.17 (9H, m), 1.61 (3H, s), 1.62 (3H, s), 1.77 (3H, s), 2.24 (1H, dd, J=14.7, 9.3 Hz), 2.43 (3H, s), 2.74 (1H, dd, J=12.5, 5.9 Hz), 2.83 (1H, dd, J=12.5, 4.3 Hz), 2.95 (1H, d, J=4.9 Hz), 3.32-3.38 (4H, m), 4.12 (1H, d, J=7.1 Hz), 4.17-4.29 (2H, m), 4.32 (1H, d, J=8.3 Hz), 4.54-4.60 (1H, m), 4.66-4.72 (1H, m), 4.87 (1H, t, J=4.8 Hz), 4.91 (1H, s), 5.20 (1H, d, J=6.9 Hz), 5.28-5.30 (1H, m), 5.99 (1H, d, J=4.9 Hz), 6.18 (1H, t, J=8.5 Hz), 7.48 (2H, t, J=7.7 Hz), 7.61 (1H, t, J=7.4 Hz), 8.15 (2H, d, J=7.6 Hz).

FAB-MS; m/z: 845 (M+H)⁺.

### Example 123

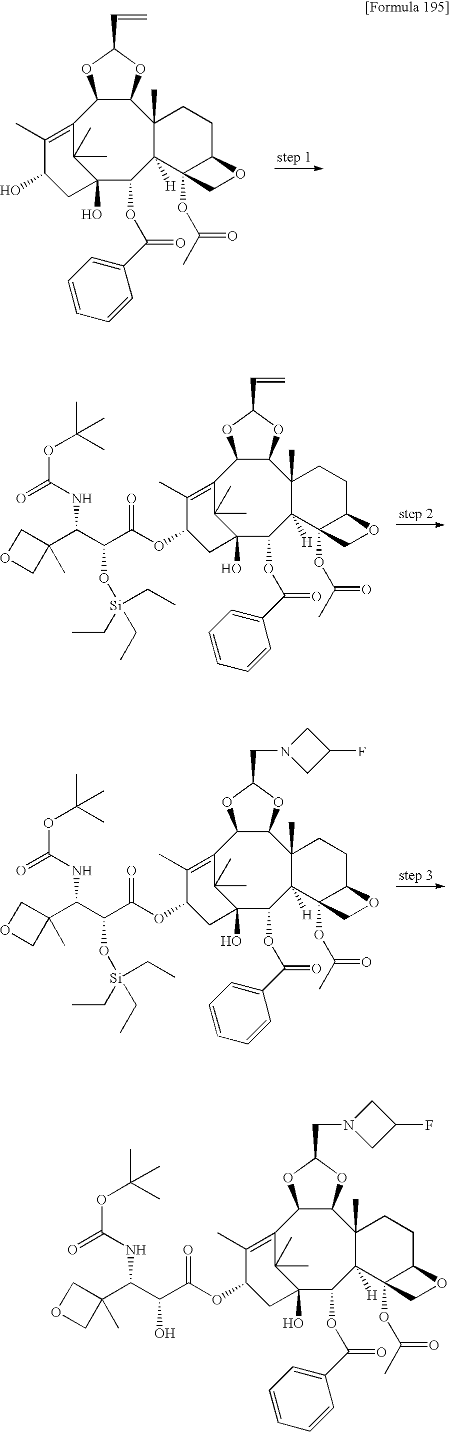

### Step 1: (1S,2S,3R,4S,5R,8R,9S,10R,13S)-4-(Acetoxy)-2-(benzoyloxy)-5,20-epoxy-1-hydroxy-9,10-[(1S)-(2-propenylidenedioxy)]tax-11-en-13-yl

### (2R,3S)-3-(tert-butoxycarbonylamino)-3-(3-methyl-3-oxetanyl)-2-[(triethylsilyl)oxy]propionate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-11-ene and the compound obtained in Reference Example 51, Step 4 were used as starting materials to perform the same procedure as that of Example 31, Step 1 and thereby to obtain the title compound as white amorphous solid.

¹H-NMR (400 MHz, CDCl₃) δ: 0.56-0.71 (6H, m), 0.98 (9H, t, J=7.8 Hz), 1.19-2.29 (7H, m), 1.30 (3H, s), 1.39 (9H, s), 1.51 (3H, s), 1.54 (3H, s), 1.64 (3H, s), 1.78 (3H, s), 2.36 (3H, s), 2.91 (1H, d, J=4.6 Hz), 3.96 (1H, dd, J=10.5, 2.0 Hz), 4.18-4.28 (4H, m), 4.33 (1H, d, J=8.3 Hz), 4.60 (1H, d, J=2.2 Hz), 4.64 (1H, d, J=5.9 Hz), 4.74 (1H, d, J=6.6 Hz), 4.92 (1H, s), 5.20 (1H, d, J=6.3 Hz), 5.26 (1H, d, J=6.8 Hz), 5.33 (1H, d, J=10.5 Hz), 5.45 (1H, d, J=10.7 Hz), 5.57 (1H, d, J=17.3 Hz), 5.94-6.06 (3H, m), 7.46-7.51 (2H, m), 7.57-7.64 (1H, m), 8.16-8.11 (2H, m).

### Step 2: (1S,2S,3R,4S,5R,8R,9S,10R,13S)-4-(Acetoxy)-2-(benzoyloxy)-5,20-epoxy-9,10-[(1S)-2-(3-fluoroazetidino)ethylidenedioxy]-1-hydroxytax-11-en-13-yl

### (2R,3S)-3-(tert-butoxycarbonylamino)-3-(3-methyl-3-oxetanyl)-2-[(triethylsilyl)oxy]propionate

The compound obtained in Step 1 mentioned above and the compound obtained in Reference Example 24, Step 2 were used as starting materials to perform the same procedure as that of Example 1 and thereby to obtain the title compound as white amorphous solid.

¹H-NMR (400 MHz, CDCl₃) δ: 0.55-0.71 (6H, m), 0.98 (9H, t, J=8.0 Hz), 1.22-2.24 (7H, m), 1.29 (3H, s), 1.39 (9H, s), 1.50 (3H, s), 1.53 (3H, s), 1.60 (3H, s), 1.76 (3H, s), 2.36 (3H, s), 2.81 (1H, dd, J=12.4, 5.6 Hz), 2.87-2.94 (2H, m), 3.25-3.40 (2H, m), 3.73-3.88 (2H, m), 3.96 (1H, d, J=10.2 Hz), 4.15 (1H, d, J=7.6 Hz), 4.18-4.29 (3H, m), 4.33 (1H, d, J=8.5 Hz), 4.60 (1H, s), 4.64 (1H, d, J=5.9 Hz), 4.74 (1H, d, J=6.3 Hz), 4.84 (1H, t, J=5.0 Hz), 4.92 (1H, brs), 5.06-5.28 (2H, m), 5.34 (1H, d, J=10.7 Hz), 5.95 (1H, d, J=4.9 Hz), 6.02 (1H, t, J=8.5 Hz), 7.49 (2H, t, J=7.6 Hz), 7.61 (1H, t, J=7.3 Hz), 8.16-8.11

(2H, m).

### Step 3: (1S,2S,3R,4S,5R,8R,9S,10R,13S)-4-(Acetoxy)-2-(benzoyloxy)-5,20-epoxy-9,10-[(1S)-2-(3-fluoroazetidino)ethylidenedioxy]-1-hydroxytax-11-en-13-yl

### (2R,3S)-3-(tert-butoxycarbonylamino)-2-hydroxy-3-(3-methyl-3-oxetanyl)propionate

The compound obtained in Step 2 mentioned above was used as a starting material to perform the same procedure as that of Example 31, Step 2 and thereby to obtain the title compound as white amorphous solid.

¹H-NMR (400 MHz, CDCl₃) δ: 1.24 (3H, s), 1.37-2.41 (7H, m), 1.41 (9H, s), 1.49 (3H, s), 1.54 (3H, s), 1.61 (3H, s), 1.81 (3H, s), 2.31 (3H, s), 2.82 (1H, dd, J=12.3, 5.9 Hz), 2.88-2.95 (2H, m), 3.26-3.40 (2H, m), 3.75-3.86 (2H, m), 4.13 (1H, d, J=7.1 Hz), 4.20-4.35 (6H, m), 4.48 (1H, s), 4.67 (1H, d, J=6.1 Hz), 4.80 (1H, d, J=6.8 Hz), 4.90 (1H, dd, J=5.4, 4.4 Hz), 4.93 (1H, brs), 5.27-5.05 (2H, m), 5.36 (1H, d, J=10.3 Hz), 6.00 (1H, d, J=4.4 Hz), 6.07 (1H, t, J=8.3 Hz), 7.48 (2H, t, J=7.8 Hz), 7.61 (1H, tt, J=7.3, 1.5 Hz), 8.15-8.10 (2H, m).

FAB-MS; m/z: 887 (M+H)⁺.

### Example 124

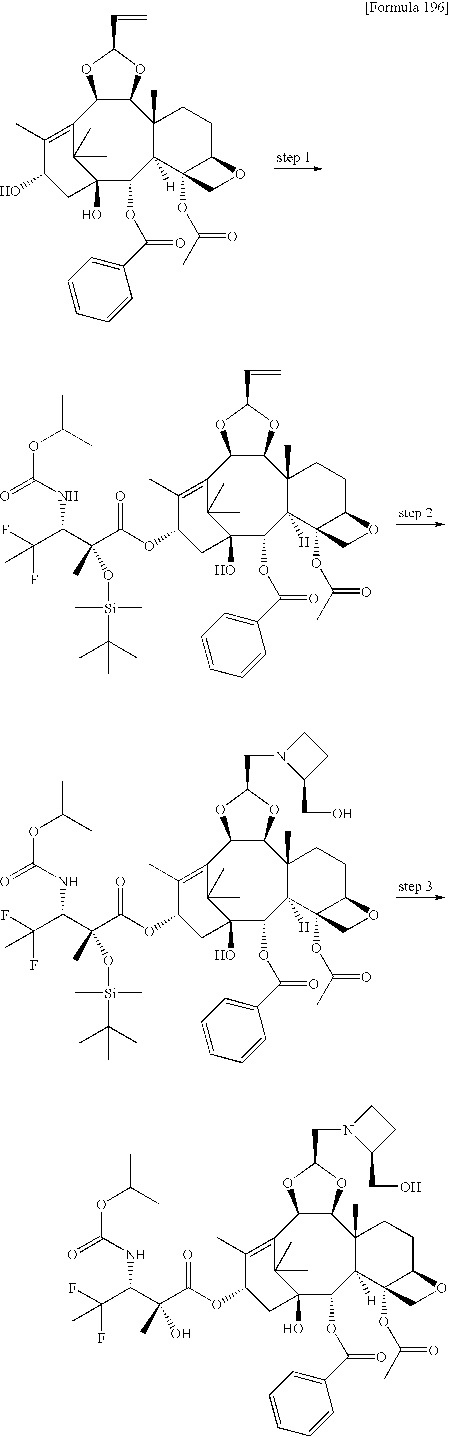

### Step 1: (1S,2S,3R,4S,5R,8R,9S,10R,13S)-4-Acetoxy-2-(benzoyloxy)-5,20-epoxy-1-hydroxy-9,10-[(1S)-2-propenylidenedioxy]tax-11-en-13-yl

### (2R,3R)-2-[(tert-butyldimethylsilyl)oxy]-4,4-difluoro-3-(isopropoxycarbonylamino)-2-methylpentanoate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-11-ene and the compound obtained in Reference Example 56 were used as starting materials to perform the same procedure as that of Example 31, Step 1 and thereby to obtain the title compound as pale yellow amorphous solid, which was used as it was for the following reaction.

### Step 2: (1S,2S,3R,4S,5R,8R,9S,10R,13S)-4-Acetoxy-2-(benzoyloxy)-5,20-epoxy-1-hydroxy-9,10-{(1S)-2-[(2S)-hydroxymethylazetidino]ethylidenedioxy}tax-11-en-13-yl

### (2R,3R)-2-[(tert-butyldimethylsilyl)oxy]-4,4-difluoro-3-(isopropoxycarbonylamino)-2-methylpentanoate

The compound obtained in Step 1 mentioned above and the compound obtained in Reference Example 25 were used as starting materials to perform the same procedure as that of Example 1 and thereby to obtain the title compound as pale yellow amorphous solid.

¹H-NMR (400 MHz, CDCl₃) δ: 0.13 (3H, s), 0.23 (3H, s), 0.94 (9H, s), 1.15 (3H, d, J=6.1 Hz), 1.20 (3H, d, J=6.1 Hz), 1.21-2.32 (11H, m), 1.32 (3H, s), 1.50 (3H, s), 1.60 (3H, s), 1.66 (3H, t, J=19.4 Hz), 1.72 (3H, s), 1.85 (3H, s), 2.45 (3H, s), 2.80-2.93 (3H, m), 3.11 (1H, q, J=8.1 Hz), 3.41-3.52 (3H, m), 3.64 (1H, dd, J=11.3, 3.2 Hz), 4.24 (1H, d, J=8.1 Hz), 4.30-4.43 (1H, m), 4.33 (1H, d, J=8.8 Hz), 4.76-4.85 (1H, m), 4.86 (1H, t, J=4.7 Hz), 4.93 (1H, s), 5.14 (1H, d, J=10.3 Hz), 5.18 (1H, d, J=7.4 Hz), 5.97 (1H, d, J=4.9 Hz), 6.10 (1H, t, J=9.1 Hz), 7.50 (2H, t, J=7.7 Hz), 7.61 (1H, t, J=7.5 Hz), 8.16 (2H, d, J=7.8 Hz).

### Step 3: (1S,2S,3R,4S,5R,8R,9S,10R,13S)-4-Acetoxy-2-(benzoyloxy)-5,20-epoxy-1-hydroxy-9,10-{(1S)-2-[(2S)-hydroxymethylazetidino]ethylidenedioxy}tax-11-en-13-yl

### (2R,3R)-4,4-difluoro-2-hydroxy-3-(isopropoxycarbonylamino)-2-methylpentanoate

The compound obtained in Step 2 mentioned above was used as a starting material to perform the same procedure as that of Example 31, Step 2 and thereby to obtain the title compound as white amorphous solid.

¹H-NMR (400 MHz, CDCl₃) δ: 1.18 (3H, d, J=6.1 Hz), 1.22 (3H, d, J=6.1 Hz), 1.28 (3H, s), 1.49 (3H, s), 1.58-2.28 (14H, m), 1.60 (3H, s), 1.67 (3H, s), 1.75 (3H, s), 2.45 (3H, s), 2.81-2.93 (3H, m), 3.11 (1H, q, J=8.3 Hz), 3.42-3.51 (3H, m), 3.66 (1H, d, J=11.5 Hz), 4.13 (1H, d, J=7.1 Hz), 4.25 (1H, d, J=8.6 Hz), 4.33 (1H, d, J=8.3 Hz), 4.47-4.56 (1H, m), 4.87-4.92 (3H, m), 5.20 (1H, d, J=6.6 Hz), 5.43 (1H, d, J=10.3 Hz), 5.98 (1H, d, J=3.7 Hz), 6.18 (1H, t, J=8.6 Hz), 7.49 (2H, t, J=7.2 Hz), 7.62 (1H, t, J=7.4 Hz), 8.15 (2H, d, J=8.3 Hz).

FAB-MS; m/z: 893 (M+H)⁺.

### Example 125

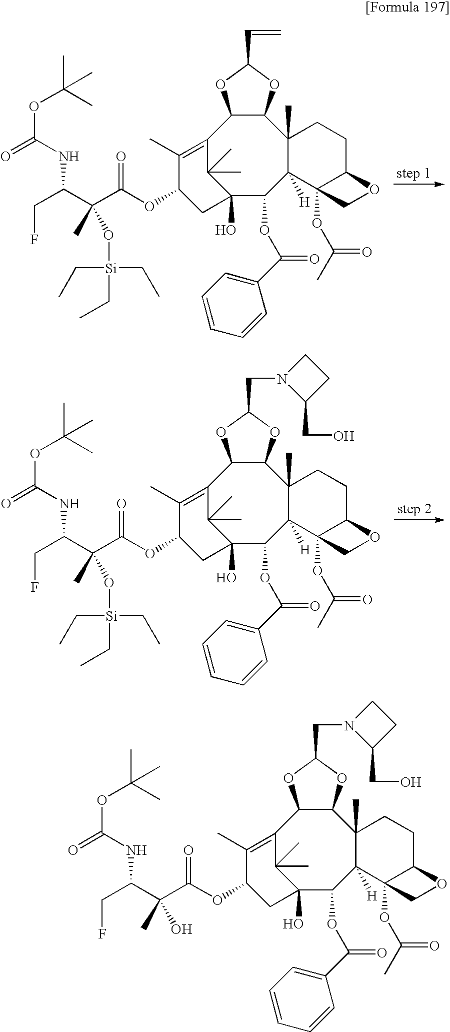

### Step 1: (1S,2S,3R,4S,5R,8R,9S,10R,13S)-4-Acetoxy-2-(benzoyloxy)-5,20-epoxy-1-hydroxy-9,10-{(11S)-2-[(2S)-hydroxymethylazetidino]ethylidenedioxy}tax-11-en-13-yl

### (2R,3R)-3-(tert-butoxycarbonylamino)-4-fluoro-2-methyl-2-[(triethylsilyl)oxy]butyrate

The compound obtained in Example 122, Step 1 and the compound obtained in Reference Example 25 were used as starting materials to perform the same procedure as that of Example 1 and thereby to obtain the title compound as pale yellow amorphous solid.

¹H-NMR (400 MHz, CDCl₃) δ: 0.61-0.78 (6H, m), 0.98 (9H, t, J=8.1 Hz), 1.31-2.27 (11H, m), 1.33 (3H, s), 1.35 (9H, s), 1.51 (3H, s), 1.60 (3H, s), 1.74 (6H, s), 2.49 (3H, s), 2.78-2.96 (3H, m), 3.10 (1H, dd, J=15.9, 8.8 Hz), 3.39-3.53 (3H, m), 3.63 (1H, dd, J=11.6, 2.9 Hz), 4.09-4.45 (4H, m), 4.57-4.73 (1H, m), 4.82-5.00 (3H, m), 5.19 (1H, d, J=7.4 Hz), 5.94 (1H, d, J=5.1 Hz), 6.13 (1H, t, J=9.3 Hz), 7.49 (2H, t, J=7.6 Hz), 7.61 (1H, t, J=7.6 Hz), 8.16 (2H, d, J=7.6 Hz).

### Step 2: (1S,2S,3R,4S,5R,8R,9S,10R,13S)-4-Acetoxy-2-(benzoyloxy)-5,20-epoxy-1-hydroxy-9,10-{(1S)-2-[(2S)-hydroxymethylazetidino]ethylidenedioxy}tax-11-en-13-yl

### (2R,3R)-3-(tert-butoxycarbonylamino)-4-fluoro-2-hydroxy-2-methylbutyrate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.28 (3H, s), 1.39-2.08 (8H, m), 1.41 (9H, s), 1.49 (3H, s), 1.60 (3H, s), 1.61 (3H, s), 1.77 (3H, s), 2.20-2.27 (3H, m), 2.43 (3H, s), 2.83 (1H, dd, J=12.7, 4.7 Hz), 2.91 (1H, dd, J=13.0, 5.1 Hz), 2.94 (1H, d, J=5.1 Hz), 3.10 (1H, dd, J=16.2, 8.8 Hz), 3.41-3.52 (3H, m), 3.65 (1H, dd, J=11.5, 2.9 Hz), 4.13 (1H, d, J=7.1 Hz), 4.19-4.28 (1H, m), 4.26 (1H, d, J=8.6 Hz), 4.32 (1H, d, J=8.6 Hz), 4.57 (1H, d, J=4.4 Hz), 4.69 (1H, d, J=4.4 Hz), 4.89 (1H, t, J=5.0 Hz), 4.91 (1H, s), 5.22 (1H, d, J=7.1 Hz), 5.31 (1H, d, J=10.5 Hz), 5.97 (1H, d, J=4.9 Hz), 6.17 (1H, t, J=8.6 Hz), 7.48 (2H, t, J=7.8 Hz), 7.61 (1H, t, J=7.6 Hz), 8.15 (2H, d, J=7.1 Hz).

FAB-MS; m/z: 875 (M+H)⁺.

### Example 126

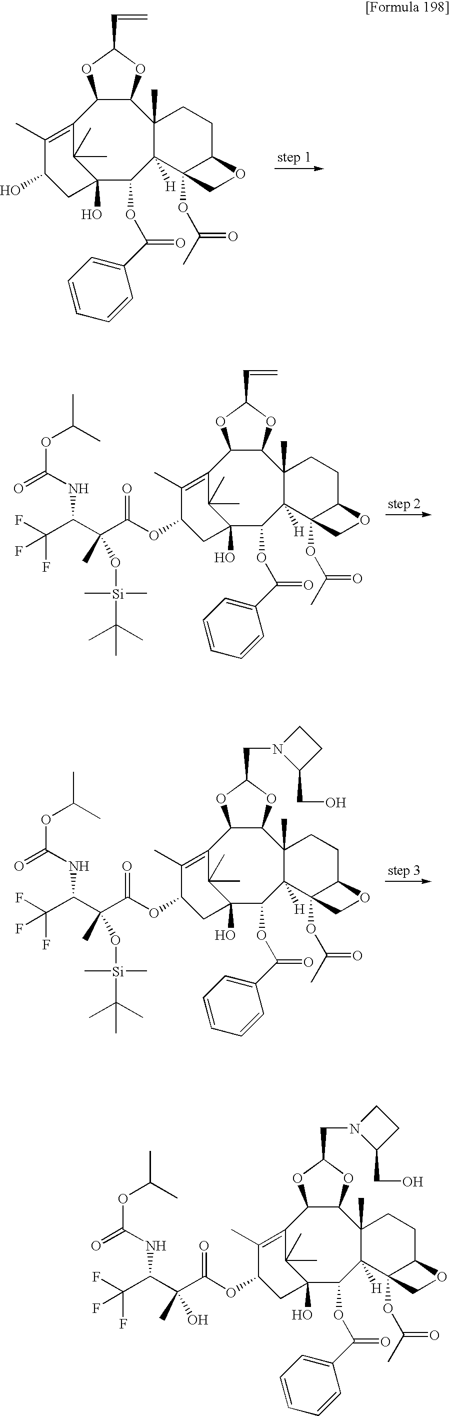

### Step 1: (1S,2S,3R,4S,5R,8R,9S,10R,13S)-4-Acetoxy-2-(benzoyloxy)-5,20-epoxy-1-hydroxy-9,10-[(1S)-2-propenylidenedioxy]tax-11-en-13-yl

### (2R,3R)-2-[(tert-butyldimethylsilyl)oxy]-3-(isopropoxycarbonylamino)-2-methyl-4,4,4-trifluorobutyrate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-11-ene and the compound obtained in Reference Example 59, Step 2 were used as starting materials to perform the same procedure as that of Example 31, Step 1 and thereby to obtain the title compound as white amorphous solid, which was used as it was for the following reaction.

### Step 2: (1S,2S,3R,4S,5R,8R,9S,10R,13S)-4-Acetoxy-2-(benzoyloxy)-5,20-epoxy-1-hydroxy-9,10-{(1S)-2-[(2S)-hydroxymethylazetidino]ethylidenedioxy}tax-11-en-13-yl

### (2R,3R)-2-[(tert-butyldimethylsilyl)oxy]-3-(isopropoxycarbonylamino)-2-methyl-4,4,4-trifluorobutyrate

The compound obtained in Step 1 mentioned above and the compound obtained in Reference Example 25 were used as starting materials to perform the same procedure as that of Example 1 and thereby to obtain the title compound as white amorphous solid.

¹H-NMR (400 MHz, CDCl₃) δ: 0.14 (3H, s), 0.24 (3H, s), 0.92 (9H, s), 1.15 (3H, d, J=6.4 Hz), 1.20 (3H, d, J=6.4 Hz), 1.32 (3H, s), 1.50 (3H, s), 1.56-2.29 (10H, m), 1.60 (3H, s), 1.72 (3H, s), 1.85 (3H, s), 2.46 (3H, s), 2.79-2.91 (3H, m), 3.10 (1H, q, J=8.3 Hz), 3.40-3.51 (3H, m), 3.63 (1H, dd, J=11.5, 2.9 Hz), 4.12 (1H, d, J=7.4 Hz), 4.24 (1H, d, J=8.3 Hz), 4.32 (1H, d, J=8.3 Hz), 4.33-4.50 (1H, m), 4.62-4.70 (1H, m), 4.79-4.87 (2H, m), 4.92 (1H, s), 5.16-5.19 (1H, m), 5.96 (1H, d, J=5.1 Hz), 6.08-6.18 (1H, m), 7.50 (2H, t, J=7.6 Hz), 7.61 (1H, t, J=7.1 Hz), 8.16 (2H, d, J=8.1 Hz).

### Step 3: (1S,2S,3R,4S,5R,8R,9S,10R,13S)-4-Acetoxy-2-(benzoyloxy)-5,20-epoxy-1-hydroxy-9,10-{(1S)-2-[(2S)-hydroxymethylazetidino]ethylidenedioxy}tax-11-en-13-yl

### (2R,3R)-2-hydroxy-3-(isopropoxycarbonylamino)-2-methyl-4,4,4-trifluorobutyrate

The compound obtained in Step 2 mentioned above was used as a starting material to perform the same procedure as that of Example 31, Step 2 and thereby to obtain the title compound as white amorphous solid.

¹H-NMR (400 MHz, CDCl₃) δ: 1.19 (3H, d, J=6.4 Hz), 1.23 (3H, d, J=6.4 Hz), 1.27 (3H, s), 1.48 (3H, s), 1.60-2.09 (8H, m), 1.60 (3H, s), 1.69 (3H, s), 1.72 (3H, s), 2.17-2.28 (2H, m), 2.43 (3H, s), 2.81-2.94 (3H, m), 3.11 (1H, dd, J=15.9, 8.0 Hz), 3.43 (1H, dd, J=11.7, 2.0 Hz), 3.46-3.53 (2H, m), 3.66 (1H, dd, J=11.3, 2.9 Hz), 4.12 (1H, d, J=7.1 Hz), 4.26 (1H, d, J=8.6 Hz), 4.33 (1H, d, J=8.6 Hz), 4.66 (1H, s), 4.76-4.96 (4H, m), 5.20 (1H, d, J=6.9 Hz), 5.50 (1H, d, J=10.3 Hz), 5.99 (1H, d, J=4.9 Hz), 6.21 (1H, t, J=8.5 Hz), 7.49 (2H, t, J=7.7 Hz), 7.62 (1H, t, J=7.2 Hz), 8.15 (2H, d, J=7.8 Hz).

FAB-MS; m/z: 897 (M+H)⁺.

### Example 127

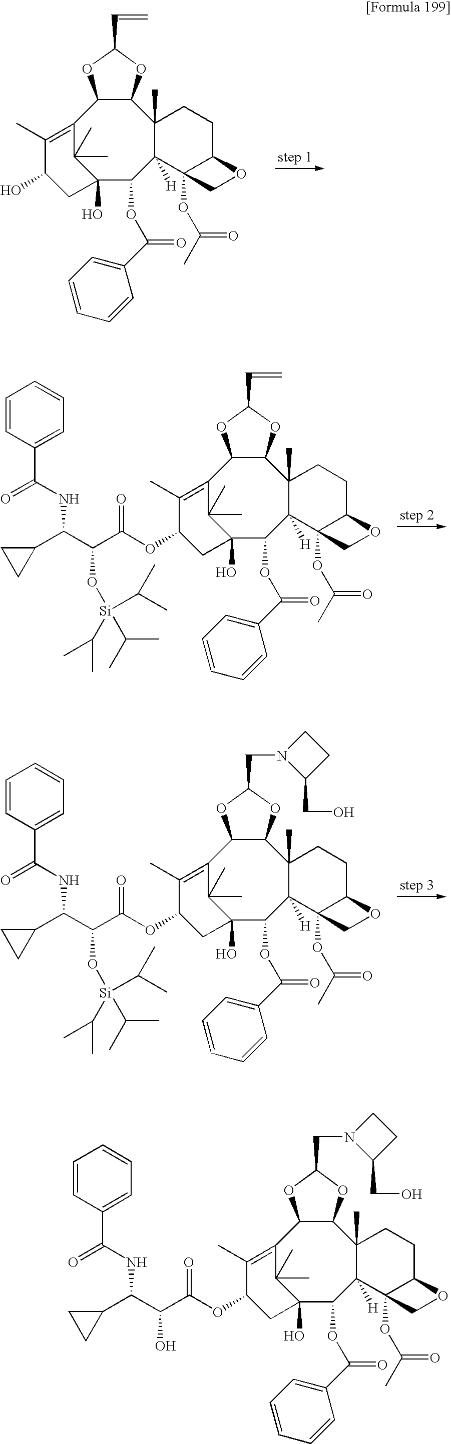

### Step 1: (1S,2S,3R,4S,5R,8R,9S,10R,13S)-4-(Acetoxy)-2-(benzoyloxy)-5,20-epoxy-1-hydroxy-9,10-[(1S)-(2-propenylidenedioxy)]tax-11-en-13-yl

### (2R,3S)-3-(benzoylamino)-3-cyclopropyl-2-[(triisopropylsilyl)oxy]butyrate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-1′-ene and the compound obtained in Reference Example 58, Step 7 were used as starting materials to perform the same procedure as that of Example 31, Step 1 and thereby to obtain the title compound as white amorphous solid.

¹H-NMR (400 MHz, CDCl₃) δ: 0.39-0.42 (1H, m), 0.62-0.69 (3H, m), 1.10-1.34 (26H, m), 1.51 (3H, s), 1.62 (3H, s), 1.78 (3H, s), 1.87-1.93 (3H, m), 2.03-2.06 (1H, m), 2.19 (2H, d, J=8.8 Hz), 2.39 (3H, s), 2.89 (1H, d, J=4.7 Hz), 3.93 (1H, td, J=9.4, 1.1 Hz), 4.20 (2H, dd, J=12.1, 7.7 Hz), 4.35 (1H, d, J=8.3 Hz), 4.81 (1H, d, J=1.5 Hz), 4.94 (1H, s), 5.19 (1H, dd, J=6.6, 2.5 Hz), 5.24 (1H, d, J=6.9 Hz), 5.45 (1H, d, J=10.3 Hz), 5.56 (1H, d, J=17.2 Hz), 5.95-6.06 (3H, m), 6.63 (1H, d, J=9.3 Hz), 7.38-7.53 (5H, m), 7.59-7.63 (1H, m), 7.70 (2H, d, J=7.1 Hz), 8.14 (2H, d, J=7.1 Hz).

ESI-MS; m/z: 978 (M+Na)⁺.

### Step 2: (1S,2S,3R,4S,5R,8R,9S,10R,13S)-4-Acetoxy-2-(benzoyloxy)-5,20-epoxy-1-hydroxy-9,10-{(1S)-2-[(2S)-hydroxymethylazetidino]ethylidenedioxy}tax-11-en-13-yl

### (2R,3S)-3-(benzoylamino)-3-cyclopropyl-2-[(triisopropylsilyl)oxy]butyrate

The compound obtained in Step 1 mentioned above and the compound obtained in Reference Example 25 were used as starting materials to perform the same procedure as that of Example 1 and thereby to obtain the title compound as white amorphous solid.

¹H-NMR (400 MHz, CDCl₃) δ: 0.38-0.41 (1H, m), 0.61-0.67 (3H, m), 1.09-1.31 (26H, m), 1.50 (3H, s), 1.56 (3H, s), 1.75 (3H, s), 1.88-2.04 (7H, m), 2.16-2.25 (2H, m), 2.38 (3H, s), 2.81-2.86 (3H, m), 3.09 (1H, dd, J=16.3, 8.2 Hz), 3.39-3.50 (3H, m), 3.62 (1H, dd, J=11.6, 3.1 Hz), 3.92 (1H, t, J=9.6 Hz), 4.13 (1H, d, J=7.4 Hz), 4.22 (1H, d, J=8.3 Hz), 4.34 (1H, d, J=8.3 Hz), 4.80 (1H, s), 4.83 (1H, t, J=4.9 Hz), 4.94 (1H, s), 5.17 (1H, d, J=7.4 Hz), 5.95 (1H, d, J=5.2 Hz), 6.02 (1H, t, J=8.7 Hz), 6.63 (1H, d, J=9.6 Hz), 7.38-7.52 (5H, m), 7.61 (1H, t, J=7.1 Hz), 7.69 (2H, d, J=7.1 Hz), 8.14 (2H, d, J=7.4 Hz).

### Step 3: (1S,2S,3R,4S,5R,8R,9S,10R,13S)-4-Acetoxy-2-(benzoyloxy)-5,20-epoxy-1-hydroxy-9,10-{(1S)-2-[(2S)-hydroxymethylazetidino]ethylidenedioxy}tax-11-en-13-yl

### (2R,3S)-3-(benzoylamino)-3-cyclopropyl-2-hydroxybutyrate

The compound obtained in Step 2 mentioned above was used as a starting material to perform the same procedure as that of Example 31, Step 2 and thereby to obtain the title compound as white amorphous solid.

¹H-NMR (400 MHz, CDCl₃) δ: 0.48-0.66 (4H, m), 1.20 (3H, s), 1.24-1.34 (1H, m), 1.48 (3H, s), 1.56 (3H, s), 1.65 (3H, s), 1.75-2.05 (8H, m), 2.20-2.35 (2H, m), 2.37 (3H, s), 2.79-2.90 (3H, m), 3.09 (1H, dd, J=16.2, 8.7 Hz), 3.39-3.51 (4H, m), 3.63 (1H, dd, J=11.5, 2.9 Hz), 4.07-4.14 (2H, m), 4.25 (1H, d, J=8.5 Hz), 4.33 (1H, d, J=8.3 Hz), 4.59 (1H, d, J=2.0 Hz), 4.84 (1H, t, J=5.0 Hz), 4.96 (1H, s), 5.14 (1H, d, J=7.3 Hz), 5.98 (1H, d, J=5.1 Hz), 6.09 (1H, t, J=8.5 Hz), 6.79 (1H, d, J=9.3 Hz), 7.41-7.53 (5H, m), 7.59-7.63 (1H, m), 7.77-7.80 (2H, m), 8.11-8.14 (2H, m).

FAB-MS; m/z: 873 (M+H)⁺.

### Example 128

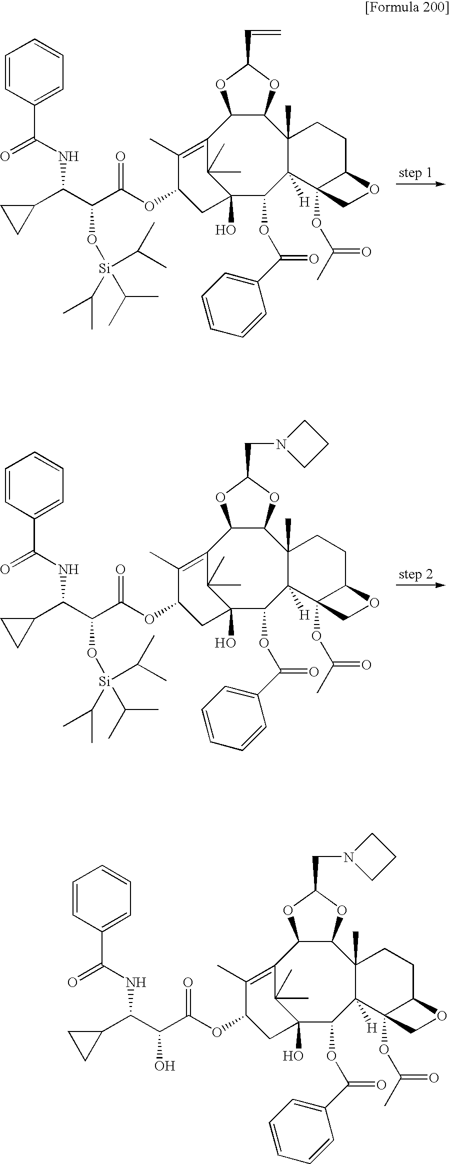

### Step 1: (1S,2S,3R,4S,5R,8R,9S,10R,13S)-4-Acetoxy-9,10-[(1S)-2-(azetidino)ethylidenedioxy]-2-(benzoyloxy)-5,20-epoxy-1-hydroxytax-11-en-13-yl

### (2R,3S)-3-(benzoylamino)-3-cyclopropyl-2-[(triisopropylsilyl)oxy]butyrate

The compound obtained in Example 127, Step 1 was used as a starting material to perform the same procedure as that of Example 1 and thereby to obtain the title compound as white amorphous solid.

¹H-NMR (400 MHz, CDCl₃) δ: 0.38-0.42 (1H, m), 0.61-0.68 (3H, m), 1.13-1.35 (26H, m), 1.50 (3H, s), 1.59 (3H, s), 1.75 (3H, s), 1.84-2.20 (8H, m), 2.38 (3H, s), 2.72 (1H, dd, J=12.5, 5.6 Hz), 2.79 (1H, dd, J=12.5, 4.4 Hz), 2.88 (1H, d, J=5.2 Hz), 3.30-3.37 (4H, m), 3.92 (1H, td, J=9.3, 1.2 Hz), 4.12 (1H, d, J=7.1 Hz), 4.22 (1H, d, J=8.3 Hz), 4.34 (1H, d, J=8.3 Hz), 4.80-4.83 (2H, m), 4.94 (1H, s), 5.15 (1H, d, J=7.1 Hz), 5.96 (1H, d, J=5.2 Hz), 6.02 (1H, t, J=9.0 Hz), 6.63 (1H, d, J=9.6 Hz), 7.38-7.52 (5H, m), 7.59-7.63 (1H, m), 7.68-7.71 (2H, m), 8.13-8.15 (2H, m).

ESI-MS; m/z: 999 (M+H)⁺.

### Step 2: (1S,2S,3R,4S,5R,8R,9S,10R,13S)-4-Acetoxy-9,10-[(1S)-2-(azetidino)ethylidenedioxy]-2-(benzoyloxy)-5,20-epoxy-1-hydroxytax-11-en-13-yl

### (2R,3S)-3-(benzoylamino)-3-cyclopropyl-2-hydroxybutyrate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 0.48-0.65 (4H, m), 1.21 (3H, s), 1.26-1.34 (1H, m), 1.48 (3H, s), 1.59 (3H, s), 1.65 (3H, s), 1.84-2.15 (8H, m), 2.31-2.35 (1H, m), 2.37 (3H, s), 2.72 (1H, dd, J=12.5, 5.6 Hz), 2.81 (1H, dd, J=12.4, 4.3 Hz), 2.90 (1H, d, J=4.9 Hz), 3.28-3.38 (4H, m), 4.06 (1H, d, J=7.1 Hz), 4.12 (1H, td, J=9.3, 1.3 Hz), 4.25 (1H, d, J=8.6 Hz), 4.34 (1H, d, J=8.6 Hz), 4.59 (1H, d, J=1.2 Hz), 4.63 (1H, brs), 4.83 (1H, t, J=5.0 Hz), 4.96 (1H, s), 5.13 (1H, d, J=6.6 Hz), 6.00 (1H, d, J=4.9 Hz), 6.09 (1H, t, J=8.2 Hz), 6.79 (1H, d, J=9.3 Hz), 7.39-7.52 (5H, m), 7.61 (1H, t, J=7.5 Hz), 7.78 (2H, d, J=7.5 Hz), 8.13 (2H, d, J=8.1 Hz).

FAB-MS; m/z: 843 (M+H)⁺.

### Example 129

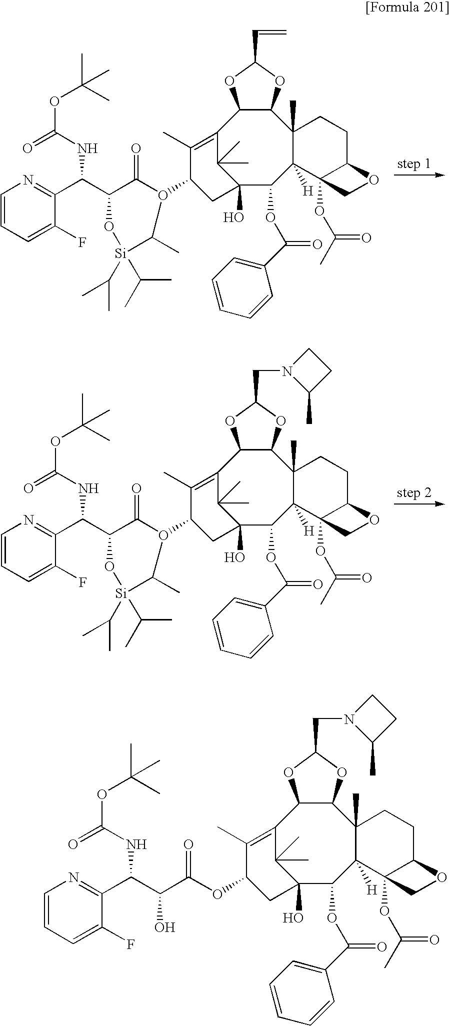

### Step 1: (1S,2S,3R,4S,5R,8R,9S,10R,13S)-4-(Acetoxy)-2-(benzoyloxy)-5,20-epoxy-1-hydroxy-9,10-{(1S)-2-[(2R)-2-methylazetidino]ethylidenedioxy}tax-11-en-13-yl

### (2R,3S)-3-(tert-butoxycarbonylamino)-3-(3-fluoro-2-pyridyl)-2-[(triisopropylsilyl)oxy]propionate

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-3-(3-fluoro-2-pyridyl)-2-[(triisopropylsilyl)oxy]propionate and the compound obtained in Reference Example 49, Step 2 were used as starting materials to perform the same procedure as that of Example 1 and thereby to obtain the title compound as white amorphous solid.

¹H-NMR (400 MHz, CDCl₃) δ: 0.86-0.93 (21H, m), 1.17-2.51 (9H, m), 1.23 (3H, d, J=6.1 Hz), 1.34 (3H, s), 1.38 (9H, s), 1.52 (3H, s), 1.63 (3H, s), 1.80 (3H, s), 2.49 (3H, s), 2.72 (1H, dd, J=12.6, 4.5 Hz), 2.86 (1H, dd, J=12.6, 5.3 Hz), 2.93-3.00 (2H, m), 3.23-3.33 (1H, m), 3.51 (1H, t, J=6.9 Hz), 4.16 (1H, d, J=8.3 Hz), 4.22 (1H, d, J=8.3 Hz), 4.35 (1H, d, J=8.3 Hz), 4.86 (1H, t, J=4.9 Hz), 4.94-4.96 (2H, m), 5.19 (1H, d, J=7.4 Hz), 5.60 (1H, d, J=9.8 Hz), 5.97 (1H, d, J=5.1 Hz), 6.05-6.12 (2H, m), 7.24-7.29 (1H, m), 7.39 (1H, t, J=9.2 Hz), 7.47 (2H, t, J=7.8 Hz), 7.59 (1H, t, J=7.5 Hz), 8.16 (2H, d, J=7.4 Hz), 8.40 (1H, d, J=4.4 Hz).

### Step 2: (1S,2S,3R,4S,5R,8R,9S,10R,13S)-4-(Acetoxy)-2-(benzoyloxy)-5,20-epoxy-1-hydroxy-9,10-{(1S)-2-[(2R)-2-methylazetidino]ethylidenedioxy}tax-11-en-13-yl

### (2R,3S)-3-(tert-butoxycarbonylamino)-3-(3-fluoro-2-pyridyl)-2-hydroxypropionate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.22-2.49 (10H, m), 1.24 (3H, d, J=5.6 Hz), 1.28 (3H, s), 1.40 (9H, s), 1.50 (3H, s), 1.62 (3H, s), 1.78 (3H, s), 2.34 (3H, s), 2.73 (1H, dd, J=12.7, 4.4 Hz), 2.87 (1H, dd, J=12.6, 5.3 Hz), 2.92-3.00 (2H, m), 3.23-3.33 (1H, m), 3.48-3.55 (1H, m), 4.14 (1H, d, J=7.1 Hz), 4.23 (1H, d, J=8.3 Hz), 4.33 (1H, d, J=8.3 Hz), 4.67 (1H, d, J=2.2 Hz), 4.88-4.93 (2H, m), 5.23 (1H, d, J=7.1 Hz), 5.65 (1H, d, J=8.8 Hz), 5.99 (1H, d, J=4.9 Hz), 6.08 (1H, t, J=8.2 Hz), 6.21 (1H, d, J=8.3 Hz), 7.28-7.33 (1H, m), 7.42-7.50 (3H, m), 7.60 (1H, t, J=7.4 Hz), 8.14 (2H, d, J=7.8 Hz), 8.40 (1H, d, J=4.7 Hz).

FAB-MS; m/z: 908 (M+H)⁺.

### Example 130

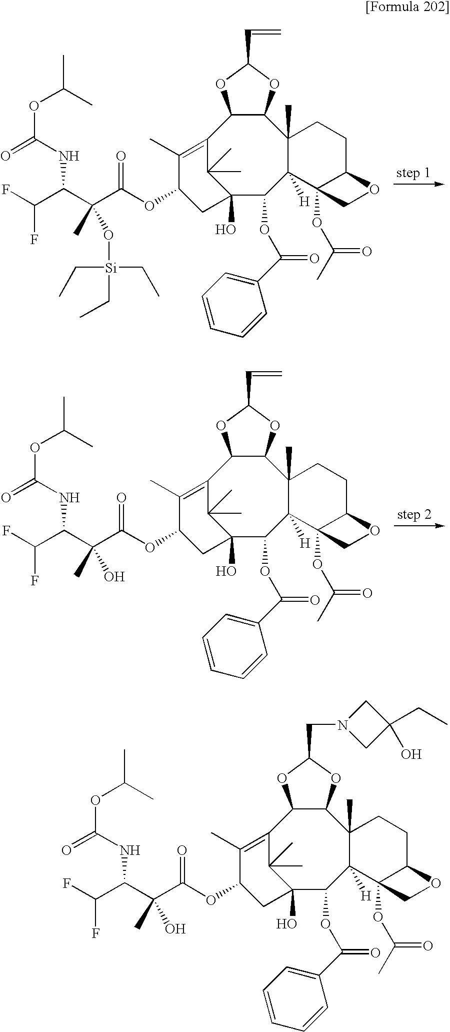

### Step 1: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3R)-4,4-difluoro-2-hydroxy-3-(isopropoxycarbonylamino)-2-methylbutyrate

The compound obtained in Example 85, Step 1 was used as a starting material to perform the same procedure as that of Example 31, Step 2 and thereby to obtain the title compound as white amorphous solid.

¹H-NMR (400 MHz, CDCl₃) δ: 1.20 (3H, d, J=7.1 Hz), 1.23 (3H, d, J=6.3 Hz), 1.29 (3H, s), 1.49 (3H, s), 1.58 (3H, s), 1.63 (3H, s), 1.77 (3H, s), 1.80-2.33 (7H, m), 2.43 (3H, s), 2.96 (1H, d, J=4.4 Hz), 4.18 (1H, d, J=6.8 Hz), 4.27 (1H, d, J=8.5 Hz), 4.33 (1H, d, J=8.3 Hz), 4.36-4.49 (1H, m), 4.66 (1H, s), 4.88-4.95 (2H, m), 5.25 (1H, d, J=6.3 Hz), 5.29 (1H, d, J=7.6 Hz), 5.44-5.49 (2H, m), 5.58 (1H, d, J=17.3 Hz), 5.93-6.12 (3H, m), 6.22 (1H, t, J=9.6 Hz), 7.48 (2H, t, J=7.7 Hz), 7.61 (1H, t, J=7.4 Hz), 8.15 (2H, d, J=8.5 Hz).

### Step 2: (1S,2S,3R,4S,5R,8R,9S,10R,13S)-4-(Acetoxy)-2-(benzoyloxy)-5,20-epoxy-9,10-[(1S)-2-(3-ethyl-3-hydroxyazetidino)ethylidenedioxy]-1-hydroxytax-11-en-13-yl

### (2R,3R)-4,4-difluoro-2-hydroxy-3-(isopropoxycarbonylamino)-2-methylbutyrate

The compound obtained in Step 1 mentioned above and the compound obtained in Reference Example 4, Step 2 were used as starting materials to perform the same procedure as that of Example 1 and thereby to obtain the title compound as white amorphous solid.

¹H-NMR (400 MHz, CDCl₃): 0.97 (3H, t, J=7.3 Hz), 1.20 (3H, d, J=6.3 Hz), 1.23 (3H, d, J=6.3 Hz), 1.28 (3H, s), 1.48 (3H, s), 1.52-2.26 (10H, m), 1.57 (3H, s), 1.62 (3H, s), 1.75 (3H, s), 2.42 (3H, s), 2.80 (1H, dd, J=12.8, 5.7 Hz), 2.88-2.97 (2H, m), 3.15 (2H, t, J=8.4 Hz), 3.46 (2H, t, J=6.8 Hz), 4.12 (1H, d, J=6.8 Hz), 4.27 (1H, d, J=8.1 Hz), 4.33 (1H, d, J=8.5 Hz), 4.37-4.50 (1H, m), 4.63 (1H, brs), 4.87-4.95 (3H, m), 5.20 (1H, d, J=6.8 Hz), 5.46 (1H, d, J=10.5 Hz), 6.00 (1H, d, J=4.6 Hz), 6.10 (1H, td, J=55.7, 3.2 Hz), 6.20 (1H, t, J=9.0 Hz), 7.49 (2H, t, J=7.8 Hz), 7.62 (1H, t, J=7.4 Hz), 8.15 (2H, d, J=8.3 Hz).

ESI-MS; m/z: 893 (M+H)⁺.

### Example 131

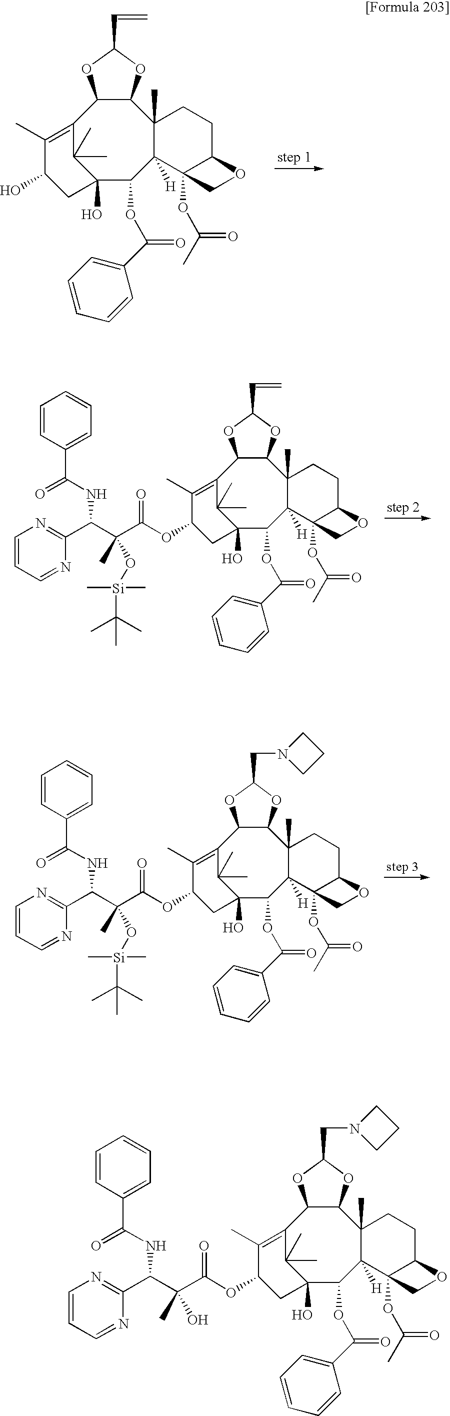

### Step 1: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3S)-3-(benzoylamino)-2-[(tert-butyldimethylsilyl)oxy]-2-methyl-3-(2-pyrimidinyl)propionate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-1′-ene and the compound obtained in Reference Example 60, Step 4 were used as starting materials to perform the same procedure as that of Example 31, Step 1 and thereby to obtain the title compound as white amorphous solid.

¹H-NMR (400 MHz, CDCl₃) δ: −0.10 (3H, s), 0.25 (3H, s), 0.81 (9H, s), 0.87-2.35 (7H, m), 1.31 (3H, s), 1.53 (3H, s), 1.65 (3H, s), 1.76 (3H, s), 1.80 (3H, s), 2.83 (3H, s), 2.93 (1H, d, J=4.9 Hz), 4.20 (1H, d, J=6.9 Hz), 4.27 (1H, d, J=8.3 Hz), 4.36 (1H, d, J=8.3 Hz), 5.01 (1H, s), 5.22 (1H, d, J=6.4 Hz), 5.27 (1H, d, J=6.9 Hz), 5.45 (1H, d, J=10.3 Hz), 5.58 (1H, d, J=17.2 Hz), 5.90 (1H, d, J=10.0 Hz), 5.96-6.06 (2H, m), 6.19 (1H, t, J=8.9 Hz), 7.21-7.25 (1H, m), 7.41 (2H, t, J=7.7 Hz), 7.45-7.51 (3H, m), 7.56-7.61 (1H, m), 7.77 (2H, d, J=8.1 Hz), 8.19 (2H, d, J=8.3 Hz), 8.72 (2H, dd, J=4.9, 1.0 Hz).

### Step 2: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3S)-3-(benzoylamino)-2-[(tert-butyldimethylsilyl)oxy]-2-methyl-3-(2-pyrimidinyl)propionate

The compound obtained in Step 1 mentioned above was used as a starting material to perform the same procedure as that of Example 1 and thereby to obtain the title compound as white amorphous solid.

¹H-NMR (400 MHz, CDCl₃) δ: −0.11 (3H, s), 0.24 (3H, s), 0.80 (9H, s), 1.24-2.35 (9H, m), 1.29 (3H, s), 1.52 (3H, s), 1.61 (3H, s), 1.75 (3H, s), 1.78 (3H, s), 2.75-2.82 (1H, m), 2.82 (3H, s), 2.86 (1H, dd, J=12.5, 4.4 Hz), 2.91 (1H, d, J=5.1 Hz), 3.38-3.44 (4H, m), 4.13 (1H, d, J=7.1 Hz), 4.27 (1H, d, J=8.3 Hz), 4.36 (1H, d, J=8.1 Hz), 4.87 (1H, t, J=4.9 Hz), 5.00 (1H, s), 5.19 (1H, d, J=7.1 Hz), 5.89 (1H, d, J=10.0 Hz), 5.99 (1H, d, J=4.7 Hz), 6.18 (1H, t, J=8.6 Hz), 7.23 (1H, t, J=4.8 Hz), 7.41 (211, t, J=7.7 Hz), 7.45-7.51 (3H, m), 7.57-7.62 (1H, m), 7.76 (2H, d, J=7.4 Hz), 8.19 (2H, d, J=8.1 Hz), 8.72 (2H, d, J=4.9 Hz).

### Step 3: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3S)-3-(benzoylamino)-2-hydroxy-2-methyl-3-(2-pyrimidinyl)propionate

The compound obtained in Step 2 mentioned above was used as a starting material to perform the same procedure as that of Example 31, Step 2 and thereby to obtain the title compound as white amorphous solid.

¹H-NMR (400 MHz, CDCl₃) δ: 1.01-2.26 (9H, m), 1.29 (3H, s), 1.50 (3H, s), 1.61 (3H, s), 1.62 (3H, s), 1.64 (3H, s), 2.61 (3H, s), 2.74 (1H, dd, J=12.5, 5.9 Hz), 2.82 (1H, dd, J=12.5, 4.4 Hz), 2.93 (1H, d, J=5.1 Hz), 3.32-3.40 (4H, m), 4.09-4.12 (1H, m), 4.24 (1H, d, J=8.3 Hz), 4.35 (1H, d, J=8.3 Hz), 4.84 (1H, t, J=5.0 Hz), 4.96 (1H, s), 5.01 (1H, brs), 5.13 (1H, d, J=7.1 Hz), 5.92 (1H, d, J=9.6 Hz), 5.98 (1H, d, J=4.9 Hz), 6.20 (1H, t, J=8.8 Hz), 7.27-7.30 (1H, m), 7.41-7.53 (5H, m), 7.58-7.63 (1H, m), 7.72 (1H, d, J=9.6 Hz), 7.86 (2H, d, J=7.4 Hz), 8.15-8.19 (2H, m), 8.76 (2H, d, J=4.9 Hz).

FAB-MS; m/z: 895 (M+H)⁺.

### Example 132

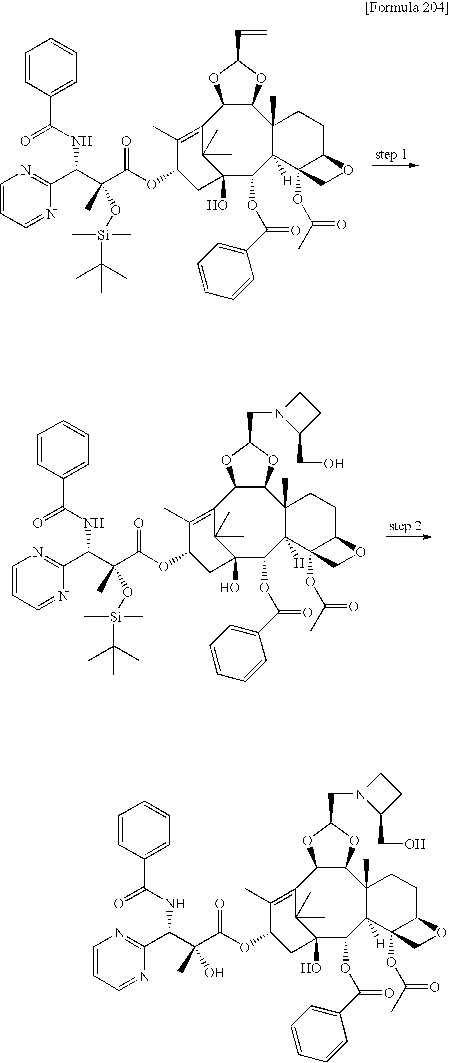

### Step 1: (1S,2S,3R,4S,5R,8R,9S,10R,13S)-4-Acetoxy-2-(benzoyloxy)-5,20-epoxy-1-hydroxy-9,10-{(1 S)-2-[(2S)-hydroxymethylazetidino]ethylidenedioxy}tax-11-en-13-yl

### (2R,3S)-3-(benzoylamino)-2-[(tert-butyldimethylsilyl)oxy]-2-methyl-3-(2-pyrimidinyl)propionate

The compound obtained in Example 131, Step 1 and the compound obtained in Reference Example 25 were used as starting materials to perform the same procedure as that of Example 1 and thereby to obtain the title compound as white amorphous solid.

¹H-NMR (400 MHz, CDCl₃) δ: −0.11 (3H, s), 0.24 (3H, s), 0.80 (9H, s), 1.20-2.34 (10H, m), 1.28 (3H, s), 1.52 (3H, s), 1.59 (3H, s), 1.75 (3H, s), 1.78 (3H, s), 2.77-2.86 (1H, m), 2.82 (3H, s), 2.90 (1H, dd, J=11.8, 4.9 Hz), 2.91 (1H, d, J=4.9 Hz), 3.07-3.15 (1H, m), 3.39-3.54 (3H, m), 3.64 (1H, dd, J=11.5, 2.7 Hz), 4.14 (1H, d, J=7.4 Hz), 4.27 (1H, d, J=8.3 Hz), 4.35 (1H, d, J=8.3 Hz), 4.86 (1H, t, J=4.9 Hz), 5.00 (1H, s), 5.20 (1H, d, J=7.1 Hz), 5.89 (1H, d, J=10.0 Hz), 5.98 (1H, d, J=4.9 Hz), 6.18 (1H, t, J=9.1 Hz), 7.23 (1H, t, J=4.9 Hz), 7.41 (2H, t, J=7.5 Hz), 7.48 (3H, t, J=6.9 Hz), 7.59 (1H, t, J=7.2 Hz), 7.76 (2H, d, J=7.4 Hz), 8.19 (2H, d, J=7.4 Hz), 8.72 (2H, d, J=4.9 Hz).

### Step 2: (1S,2S,3R,4S,5R,8R,9S,10R,13S)-4-Acetoxy-2-(benzoyloxy)-5,20-epoxy-1-hydroxy-9,10-{(1S)-2-[(2S)-hydroxymethylazetidino]ethylidenedioxy}tax-11-en-13-yl

### (2R,3S)-3-(benzoylamino)-2-hydroxy-2-methyl-3-(2-pyrimidinyl)propionate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.22-2.27 (10H, m), 1.28 (3H, s), 1.50 (3H, s), 1.59 (3H, s), 1.63 (3H, s), 1.63 (3H, s), 2.62 (3H, s), 2.82 (1H, dd, J=13.0, 4.7 Hz), 2.88 (1H, dd, J=12.7, 5.1 Hz), 2.93 (1H, d, J=4.9 Hz), 3.06-3.14 (1H, m), 3.42 (1H, dd, J=11.5, 2.7 Hz), 3.45-3.52 (2H, m), 3.64 (1H, dd, J=11.4, 3.1 Hz), 4.12 (1H, d, J=7.4 Hz), 4.24 (1H, d, J=8.3 Hz), 4.35 (1H, d, J=8.1 Hz), 4.85 (1H, t, J=4.9 Hz), 4.93-5.01 (2H, m), 5.15 (1H, d, J=7.1 Hz), 5.92 (1H, d, J=9.6 Hz), 5.97 (1H, d, J=5.1 Hz), 6.19 (1H, t, J=8.8 Hz), 7.28 (1H, t, J=4.9 Hz), 7.41-7.54 (5H, m), 7.58-7.63 (1H, m), 7.72 (1H, d, J=9.8 Hz), 7.84-7.87 (2H, m), 8.15-8.18 (2H, m), 8.76 (2H, d, J=4.9 Hz).

FAB-MS; m/z: 925 (M+H)⁺.

### Example 133

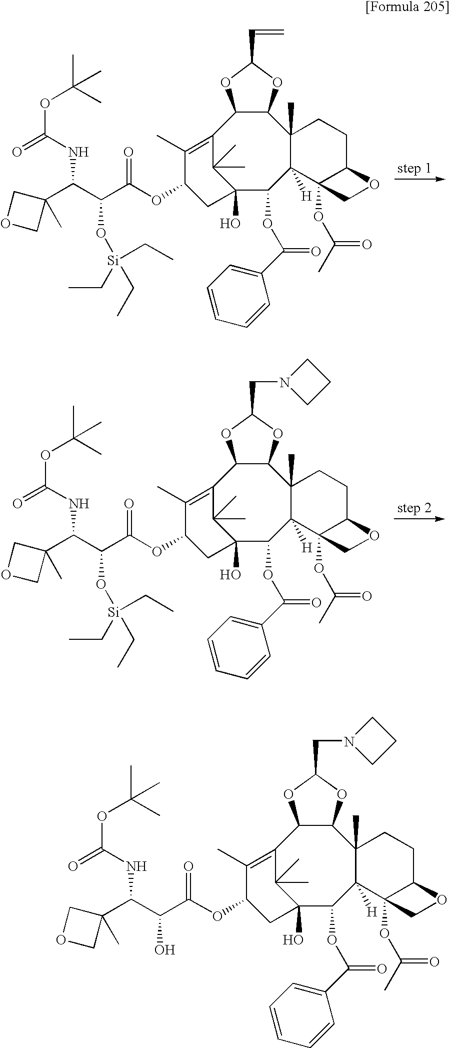

### Step 1: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3S)-3-(tert-butoxycarbonylamino)-3-(3-methyl-3-oxetanyl)-2-[(triethylsilyl)oxy]propionate

The compound obtained in Example 123, Step 1 was used as a starting material to perform the same procedure as that of Example 1 and thereby to obtain the title compound as white amorphous solid.

¹H-NMR (400 MHz, CDCl₃) δ: 0.55-0.71 (6H, m), 0.98 (9H, t, J=8.1 Hz), 1.10-2.37 (9H, m), 1.28 (3H, s), 1.38 (9H, s), 1.50 (3H, s), 1.53 (3H, s), 1.60 (3H, s), 1.75 (3H, s), 2.35 (3H, s), 2.80-2.99 (3H, m), 3.53 (4H, brs), 3.95 (1H, dd, J=10.4, 1.8 Hz), 4.14 (1H, d, J=7.11 Hz), 4.20 (1H, d, J=8.5 Hz), 4.25 (2H, dd, J=8.2, 6.3 Hz), 4.33 (1H, d, J=8.2 Hz), 4.59 (1H, d, J=2.0 Hz), 4.63 (1H, d, J=5.9 Hz), 4.74 (1H, d, J=6.6 Hz), 4.91 (2H, brs), 5.17 (1H, d, J=7.3 Hz), 5.34 (1H, d, J=10.5 Hz), 5.95 (1H, d, J=5.1 Hz), 6.01 (1H, t, J=8.9 Hz), 7.49 (2H, t, J=7.7 Hz), 7.61 (1H, tt, J=7.4, 1.2 Hz), 8.16-8.10 (2H, m).

### Step 2: (1S,2S,3R,4S,5R,8R,9S,10R,13S)-4-(Acetoxy)-9,10-[(1S)-2-(azetidino)ethylidenedioxy]-2-(benzoyloxy)-5,20-epoxy-1-hydroxytax-11-en-13-yl

### (2R,3S)-3-(tert-butoxycarbonylamino)-2-hydroxy-3-(3-methyl-3-oxetanyl)propionate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.12-2.40 (9H, m), 1.23 (3H, s), 1.41 (9H, s), 1.49 (3H, s), 1.54 (3H, s), 1.60 (3H, s), 1.80 (3H, s), 2.31 (3H, s), 2.84 (3H, m), 3.37-3.46 (4H, m), 3.48-3.67 (1H, m), 4.12 (1H, d, J=7.1 Hz), 4.19-4.34 (5H, m), 4.48 (1H, s), 4.67 (1H, d, J=6.1 Hz), 4.80 (1H, d, J=6.8 Hz), 4.89-4.95 (2H, m), 5.23 (1H, d, J=6.8 Hz), 5.37 (1H, d, J=10.0 Hz), 6.00 (1H, d, J=5.1 Hz), 6.06 (1H, t, J=8.0 Hz), 7.48 (2H, t, J=7.8 Hz), 7.61 (1H, tt, J=7.4, 1.2 Hz), 8.09-8.14 (2H, m).

FAB-MS; m/z: 869 (M+H)⁺.

### Example 134

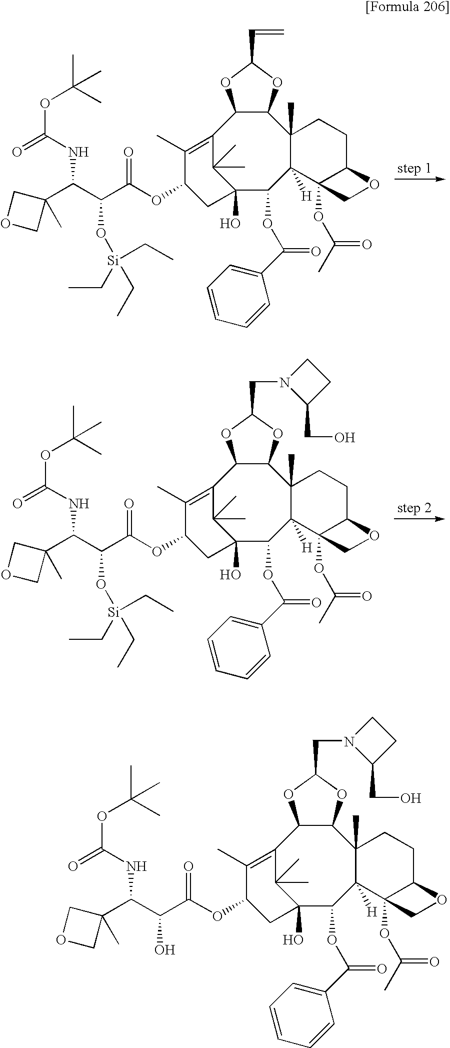

### Step 1: (1S,2S,3R,4S,5R,8R,9S,10R,13S)-4-Acetoxy-2-(benzoyloxy)-5,20-epoxy-1-hydroxy-9,10-{(1S)-2-[(2S)-hydroxymethylazetidino]ethylidenedioxy}tax-11-en-13-yl

### (2R,3S)-3-(tert-butoxycarbonylamino)-3-(3-methyl-3-oxetanyl)-2-[(triethylsilyl)oxy]propionate

The compound obtained in Example 123, Step 1 and the compound obtained in Reference Example 25 were used as starting materials to perform the same procedure as that of Example 1 and thereby to obtain the title compound as white amorphous solid.

¹H-NMR (400 MHz, CDCl₃) δ: 0.55-0.71 (6H, m), 0.95-2.29 (10H, m), 0.98 (9H, t, J=7.8 Hz), 1.28 (3H, s), 1.38 (9H, s), 1.50 (3H, s), 1.53 (3H, s), 1.58 (3H, s), 1.76 (3H, s), 2.35 (3H, s), 2.82 (1H, dd, J=12.7, 4.6 Hz), 2.89 (1H, d, J=5.1 Hz), 2.91 (1H, dd, J=12.7, 4.6 Hz), 3.12 (1H, q, J=8.3 Hz), 3.44 (1H, dd, J=11.7, 2.8 Hz), 3.47-3.55 (2H, m), 3.64 (1H, dd, J=11.7, 2.8 Hz), 3.95 (1H, dd, J=10.5, 1.7 Hz), 4.15 (1H, d, J=7.3 Hz), 4.20 (1H, d, J=8.3 Hz), 4.25 (2H, dd, J=8.3, 6.3 Hz), 4.32 (1H, d, J=8.1 Hz), 4.59 (1H, d, J=2.2 Hz), 4.63 (1H, d, J=5.9 Hz), 4.74 (1H, d, J=6.8 Hz), 4.85 (1H, t, J=4.9 Hz), 4.91 (1H, brs), 5.18 (1H, d, J=7.1 Hz), 5.33 (1H, d, J=10.3 Hz), 5.94 (1H, d, J=5.1 Hz), 6.01 (1H, t, J=8.9 Hz), 7.49 (2H, t, J=7.7 Hz), 7.60 (1H, tt, J=7.4, 1.5 Hz), 8.15-8.11 (2H, m).

### Step 2: (1S,2S,3R,4S,5R,8R,9S,10R,13S)-4-Acetoxy-2-(benzoyloxy)-5,20-epoxy-1-hydroxy-9,10-{(1S)-2-[(2S)-hydroxymethylazetidino]ethylidenedioxy}tax-11-en-13-yl

### (2R,3S)-3-(tert-butoxycarbonylamino)-2-hydroxy-3-(3-methyl-3-oxetanyl)propionate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.22-2.40 (10H, m), 1.23 (3H, s), 1.41 (9H, s), 1.49 (3H, s), 1.54 (3H, s), 1.57 (3H, s), 1.80 (3H, s), 2.31 (3H, s), 2.86-2.93 (2H, m), 3.00 (1H, dd, J=12.8, 4.8 Hz), 3.19 (1H, q, J=8.4 Hz), 3.46-3.63 (4H, m), 3.66-3.72 (1H, m), 4.13 (1H, d, J=7.1 Hz), 4.18-4.34 (5H, m), 4.48 (1H, s), 4.67 (1H, d, J=5.9 Hz), 4.80 (1H, d, J=6.8 Hz), 4.90-4.96 (2H, m), 5.24 (1H, d, J=7.1 Hz), 5.38 (1H, d, J=10.2 Hz), 5.98 (1H, d, J=4.9 Hz), 6.06 (1H, t, J=8.3 Hz), 7.47 (2H, t, J=7.7 Hz), 7.60 (1H, tt, J=7.3, 1.2 Hz), 8.13-8.09 (2H, m).

FAB-MS; m/z: 899 (M+H)⁺.

### Example 135

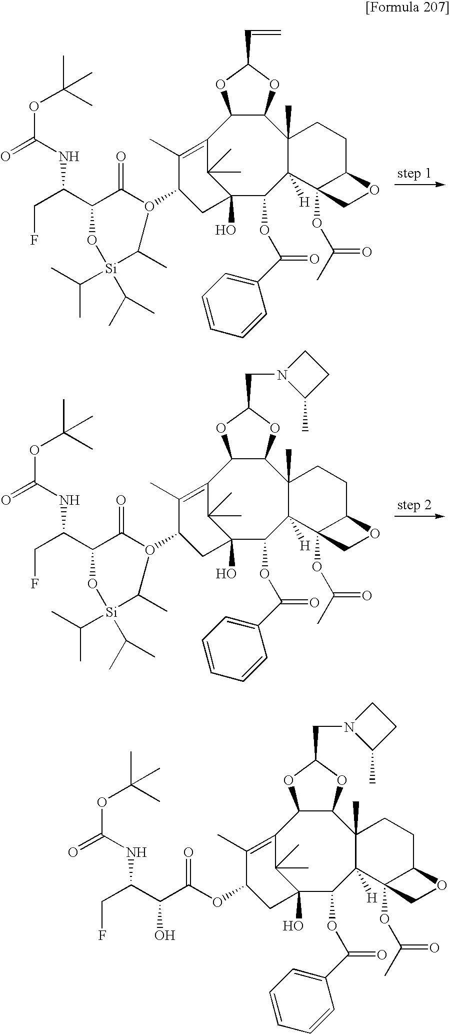

### Step 1: (1S,2S,3R,4S,5R,8R,9S,10R,13S)-4-(Acetoxy)-2-(benzoyloxy)-5,20-epoxy-1-hydroxy-9,10-{(1S)-2-[(2S)-2-methylazetidino]ethylidenedioxy}tax-11-en-13-yl

### (2R,3R)-3-(tert-butoxycarbonylamino)-4-fluoro-2-[(triisopropylsilyl)oxy]butyrate

The compound obtained in Example 34, Step 1 and the compound obtained in Reference Example 61, Step 3 were used as starting materials to perform the same procedure as that of Example 1 and thereby to obtain the title compound as white amorphous solid.

¹H-NMR (400 MHz, CDCl₃) δ: 1.08-2.34 (9H, m), 1.09-1.14 (21H, m), 1.21 (3H, d, J=5.9 Hz), 1.26 (3H, s), 1.37 (9H, s), 1.50 (3H, s), 1.61 (3H, s), 1.76 (3H, s), 2.32 (3H, s), 2.62 (1H, dd, J=12.6, 6.3 Hz), 2.88-2.95 (3H, m), 3.21-3.30 (1H, m), 3.50 (1H, t, J=7.1 Hz), 4.12 (1H, d, J=7.1 Hz), 4.19 (1H, d, J=8.3 Hz), 4.30-4.48 (4H, m), 4.76-4.80 (2H, m), 4.84-4.88 (1H, m), 4.91 (1H, s), 5.17 (1H, d, J=7.1 Hz), 5.96 (1H, d, J=5.1 Hz), 6.01 (1H, t, J=8.6 Hz), 7.49 (2H, t, J=7.6 Hz), 7.57-7.63 (1H, m), 8.14 (2H, d, J=8.3 Hz).

### Step 2: (1S,2S,3R,4S,5R,8R,9S,10R,13S)-4-(Acetoxy)-2-(benzoyloxy)-5,20-epoxy-1-hydroxy-9,10-{(1S)-2-[(2S)-2-methylazetidino]ethylidenedioxy}tax-11-en-13-yl

### (2R,3R)-3-(tert-butoxycarbonylamino)-4-fluoro-2-hydroxybutyrate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.20-2.41 (10H, m), 1.22 (3H, d, J=6.4 Hz), 1.23 (3H, s), 1.41 (9H, s), 1.49 (3H, s), 1.61 (3H, s), 1.78 (3H, s), 2.33 (3H, s), 2.64 (1H, dd, J=12.5, 6.4 Hz), 2.89-2.96 (3H, m), 3.22-3.31 (1H, m), 3.48-3.54 (1H, m), 4.11 (1H, d, J=7.1 Hz), 4.24 (1H, d, J=8.6 Hz), 4.33 (1H, d, J=8.3 Hz), 4.39-4.65 (4H, m), 4.88-4.92 (1H, m), 4.94 (1H, s), 5.12 (1H, d, J=9.1 Hz), 5.23 (1H, d, J=6.9 Hz), 6.01 (1H, d, J=4.9 Hz), 6.06 (1H, t, J=7.6 Hz), 7.48 (2H, t, J=7.7 Hz), 7.59-7.64 (1H, m), 8.11-8.15 (2H, m).

ESI-MS; m/z: 845 (M+H)⁺.

### Example 136

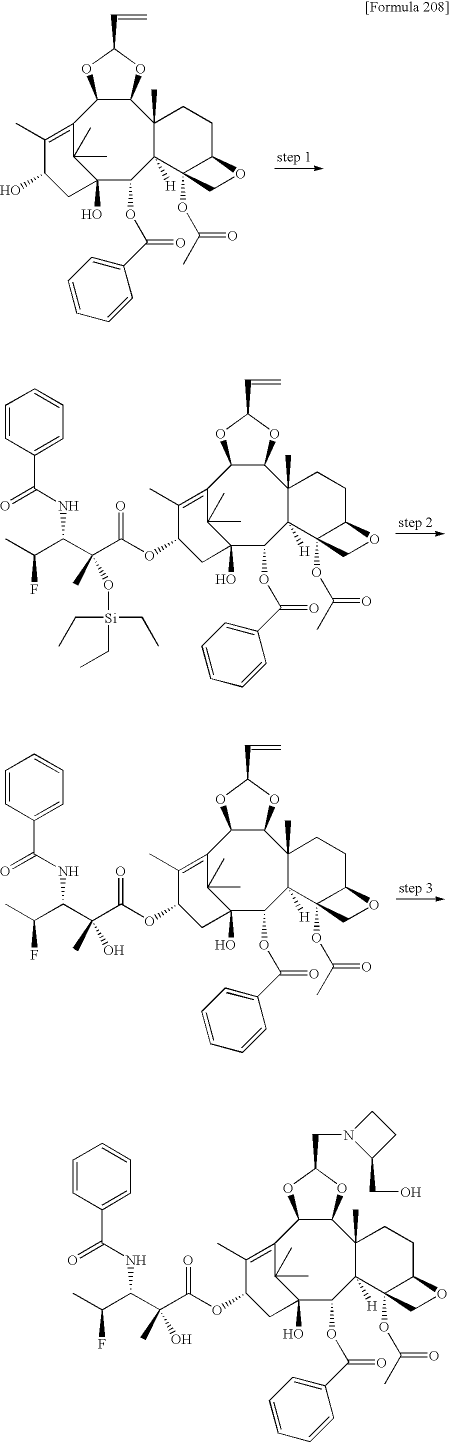

### Step 1: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3R,4S)-3-(benzoylamino)-4-fluoro-2-methyl-2-[(triethylsilyl)oxy]pentanoate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-11-ene and the compound obtained in Reference Example 62, Step 4 were used as starting materials to perform the same procedure as that of Example 31, Step 1 and thereby to obtain the title compound as white amorphous solid, which was used as it was for the following reaction.

### Step 2: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl

### (2R,3R,4S)-3-(benzoylamino)-4-fluoro-2-hydroxy-2-methylpentanoate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.26 (3H, s), 1.48 (3H, dd, J=24.4, 7.3 Hz), 1.49 (3H, s), 1.56-2.17 (7H, m), 1.59 (3H, s), 1.62 (3H, s), 1.67 (3H, s), 2.54 (3H, s), 2.91 (1H, d, J=4.9 Hz), 4.14 (1H, d, J=7.6 Hz), 4.21 (1H, brs), 4.25 (1H, d, J=8.8 Hz), 4.34 (1H, d, J=8.8 Hz), 4.82-5.01 (2H, m), 4.95 (1H, s), 5.21 (2H, d, J=6.3 Hz), 5.45 (1H, d, J=10.7 Hz), 5.56 (1H, d, J=17.6 Hz), 5.94-6.05 (2H, m), 6.15 (1H, t, J=8.8 Hz), 6.72 (1H, d, J=10.0 Hz), 7.41-7.65 (6H, m), 7.72 (2H, d, J=8.5 Hz), 8.17 (2H, d, J=7.8 Hz).

### Step 3: (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-11-en-13-yl

### (2R,3R,4S)-3-(benzoylamino)-4-fluoro-2-hydroxy-2-methylpentanoate

The compound obtained in Step 2 mentioned above and the compound obtained in Reference Example 52 were used as starting materials to perform the same procedure as that of Example 1 and thereby to obtain the title compound as white amorphous solid.

¹H-NMR (400 MHz, CDCl₃) δ: 1.22 (3H, s), 1.48 (3H, dd, J=24.6, 6.1 Hz), 1.48 (3H, s), 1.57 (3H, s), 1.59-2.29 (10H, m), 1.66 (3H, s), 1.71 (3H, s), 2.54 (3H, s), 2.81 (1H, dd, J=13.4, 4.6 Hz), 2.84-2.91 (2H, m), 3.09 (1H, dd, J=16.3, 8.3 Hz), 3.38-3.52 (3H, m), 3.63 (1H, dd, J=11.2, 2.9 Hz), 4.08-4.26 (1H, m), 4.09 (1H, d, J=7.8 Hz), 4.25 (1H, d, J=8.5 Hz), 4.33 (1H, d, J=8.0 Hz), 4.81-5.00 (1H, m), 4.85 (1H, t, J=6.1 Hz), 4.94 (1H, brs), 5.07-5.16 (2H, m), 5.96 (1H, d, J=5.9 Hz), 6.13 (1H, t, J=8.7 Hz), 6.70 (1H, d, J=10.0 Hz), 7.42 (2H, t, J=7.3 Hz), 7.47-7.54 (3H, m), 7.62 (1H, t, J=7.8 Hz), 7.72 (2H, d, J=7.3 Hz), 8.17 (2H, d, J=7.8 Hz).

ESI-MS; m/z: 893 (M+H)⁺.

### Example 137

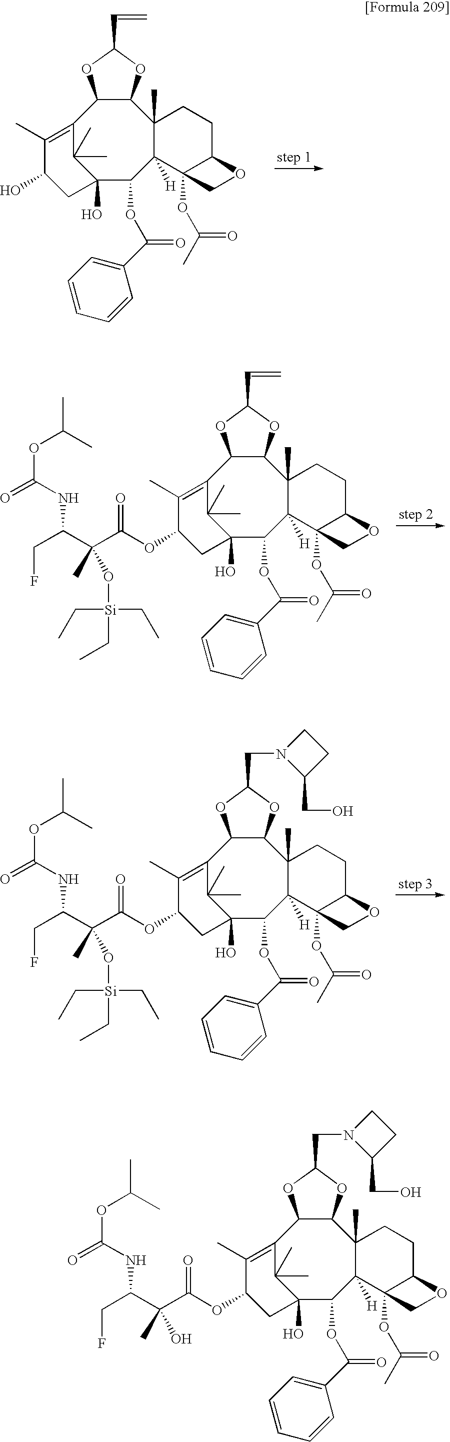

### Step 1: (1S,2S,3R,4S,5R,8R,9S,10R,13S)-4-(Acetoxy)-2-(benzoyloxy)-5,20-epoxy-1-hydroxy-9,10-[(1S)-(2-propenylidenedioxy)]tax-11-en-13-yl

### (2R,3R)-4-fluoro-3-(isopropoxycarbonylamino)-2-methyl-2-[(triethylsilyl)oxy]butyrate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-11-ene and the compound obtained in Reference Example 63 were used as starting materials to perform the same procedure as that of Example 31, Step 1 and thereby to obtain the title compound as white amorphous solid.

¹H-NMR (400 MHz, CDCl₃) δ: 0.62-0.80 (6H, m), 0.99 (9H, t, J=7.8 Hz), 1.15 (6H, dd, J=15.9, 6.4 Hz), 1.21-2.21 (7H, m), 1.35 (3H, s), 1.51 (3H, s), 1.66 (3H, s), 1.76 (6H, s), 2.49 (3H, s), 2.89 (1H, d, J=5.1 Hz), 4.18 (1H, d, J=7.4 Hz), 4.23 (1H, d, J=8.3 Hz), 4.31-4.84 (5H, m), 4.89 (1H, d, J=9.3 Hz), 4.92 (1H, s), 5.22 (1H, d, J=5.9 Hz), 5.26 (1H, d, J=6.9 Hz), 5.46 (1H, d, J=10.5 Hz), 5.58 (1H, d, J=17.4 Hz), 5.96-6.04 (2H, m), 6.14 (1H, t, J=8.8 Hz), 7.49 (2H, t, J=7.7 Hz), 7.61 (1H, t, J=7.4 Hz), 8.17 (2H, d, J=7.8 Hz).

### Step 2: (1S,2S,3R,4S,5R,8R,9S,10R,13S)-4-Acetoxy-2-(benzoyloxy)-5,20-epoxy-1-hydroxy-9,10-{(11S)-2-[(2S)-hydroxymethylazetidino]ethylidenedioxy}tax-11-en-13-yl

### (2R,3R)-4-fluoro-3-(isopropoxycarbonylamino)-2-methyl-2-[(triethylsilyl)oxy]butyrate

The compound obtained in Step 1 mentioned above and the compound obtained in Reference Example 25 were used as starting materials to perform the same procedure as that of Example 1 and thereby to obtain the title compound as pale yellow amorphous solid, which was used as it was for the following reaction.

### Step 3: (1S,2S,3R,4S,5R,8R,9S,10R,13S)-4-Acetoxy-2-(benzoyloxy)-5,20-epoxy-1-hydroxy-9,10-{(1S)-2-[(2S)-hydroxymethylazetidino]ethylidenedioxy}tax-11-en-13-yl

### (2R,3R)-4-fluoro-2-hydroxy-3-(isopropoxycarbonylamino)-2-methylbutyrate

The compound obtained in Step 2 mentioned above was used as a starting material to perform the same procedure as that of Example 31, Step 2 and thereby to obtain the title compound as white amorphous solid.

¹H-NMR (400 MHz, CDCl₃) δ: 1.19 (3H, d, J=6.6 Hz), 1.22 (3H, d, J=6.6 Hz), 1.27 (3H, s), 1.49 (3H, s), 1.57-2.06 (8H, m), 1.60 (6H, s), 1.76 (3H, s), 2.20-2.30 (2H, m), 2.42 (3H, s), 2.83 (1H, dd, J=12.7, 4.2 Hz), 2.91 (1H, dd, J=12.7, 5.0 Hz), 2.95 (1H, d, J=4.7 Hz), 3.11 (1H, q, J=8.2 Hz), 3.43 (1H, d, J=12.0 Hz), 3.46-3.53 (2H, m), 3.65 (1H, d, J=9.8 Hz), 4.13 (1H, d, J=6.9 Hz), 4.23-4.35 (1H, m), 4.26 (1H, d, J=8.6 Hz), 4.33 (1H, d, J=8.3 Hz), 4.47 (1H, brs), 4.56-4.61 (1H, m), 4.67-4.73 (1H, m), 4.85-4.94 (3H, m), 5.22 (1H, d, J=7.1 Hz), 5.40 (1H, d, J=9.8 Hz), 5.99 (1H, d, J=4.4 Hz), 6.18 (1H, t, J=8.5 Hz), 7.48 (2H, t, J=7.4 Hz), 7.61 (1H, t, J=7.4 Hz), 8.15 (2H, d, J=7.8 Hz).

FAB-MS; m/z: 861 (M+H)⁺.

### Example 138

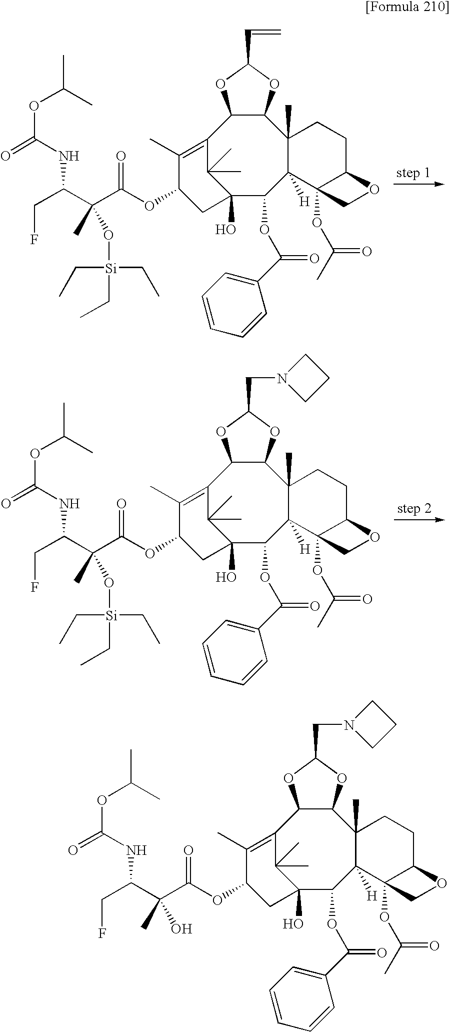

### Step 1: (1S,2S,3R,4S,5R,8R,9S,10R,13S)-4-Acetoxy-9,10-[(1S)-2-(azetidino)ethylidenedioxy]-2-(benzoyloxy)-5,20-epoxy-1-hydroxytax-1′-en-13-yl

### (2R,3R)-4-fluoro-3-(isopropoxycarbonylamino)-2-methyl-2-[(triethylsilyl)oxy]butyrate

The compound obtained in Example 137, Step 1 was used as a starting material to perform the same procedure as that of Example 1 and thereby to obtain the title compound as pale yellow amorphous solid.

¹H-NMR (400 MHz, CDCl₃) δ: 0.61-0.78 (6H, m), 0.98 (9H, t, J=7.6 Hz), 1.13 (3H, d, J=6.1 Hz), 1.17 (3H, d, J=6.4 Hz), 1.33 (3H, s), 1.50 (3H, s), 1.58-2.2 (9H, m), 1.62 (3H, s), 1.73 (3H, s), 1.75 (3H, s), 2.49 (3H, s), 2.74 (1H, dd, J=12.3, 5.9 Hz), 2.81 (1H, dd, J=12.7, 5.0 Hz), 2.87 (1H, d, J=4.9 Hz), 3.31-3.39 (4H, m), 4.11 (1H, d, J=7.4 Hz), 4.23 (1H, d, J=8.3 Hz), 4.31-4.46 (3H, m), 4.59-4.91 (5H, m), 5.17 (1H, d, J=7.4 Hz), 5.97 (1H, d, J=5.1 Hz), 6.13 (1H, t, J=8.9 Hz), 7.49 (2H, t, J=7.6 Hz), 7.58-7.63 (1H, m), 8.17 (2H, d, J=7.8 Hz).

### Step 2: (1S,2S,3R,4S,5R,8R,9S,10R,13S)-4-Acetoxy-9,10-[(1S)-2-(azetidino)ethylidenedioxy]-2-(benzoyloxy)-5,20-epoxy-1-hydroxytax-11-en-13-yl

### (2R,3R)-4-fluoro-2-hydroxy-3-(isopropoxycarbonylamino)-2-methylbutyrate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.18 (3H, d, J=6.4 Hz), 1.21 (3H, d, J=6.1 Hz), 1.27 (3H, s), 1.48 (3H, s), 1.56-2.25 (10H, m), 1.60 (3H, s), 1.62 (3H, s), 1.76 (3H, s), 2.42 (3H, s), 2.74 (1H, dd, J=12.3, 5.9 Hz), 2.83 (1H, dd, J=12.3, 4.4 Hz), 2.95 (1H, d, J=4.9 Hz), 3.33-3.40 (4H, m), 4.11 (1H, d, J=7.1 Hz), 4.23-4.34 (1H, m), 4.26 (1H, d, J=8.3 Hz), 4.32 (1H, d, J=8.6 Hz), 4.55-4.60 (1H, m), 4.67-4.72 (1H, m), 4.84-4.93 (3H, m), 5.20 (1H, d, J=7.1 Hz), 5.41 (1H, d, J=10.0 Hz), 6.00 (1H, d, J=4.9 Hz), 6.18 (1H, t, J=8.3 Hz), 7.48 (2H, t, J=7.6 Hz), 7.59-7.63 (1H, m), 8.15 (2H, d, J=7.8 Hz).

FAB-MS; m/z: 831 (M+H)⁺.

### Example 139

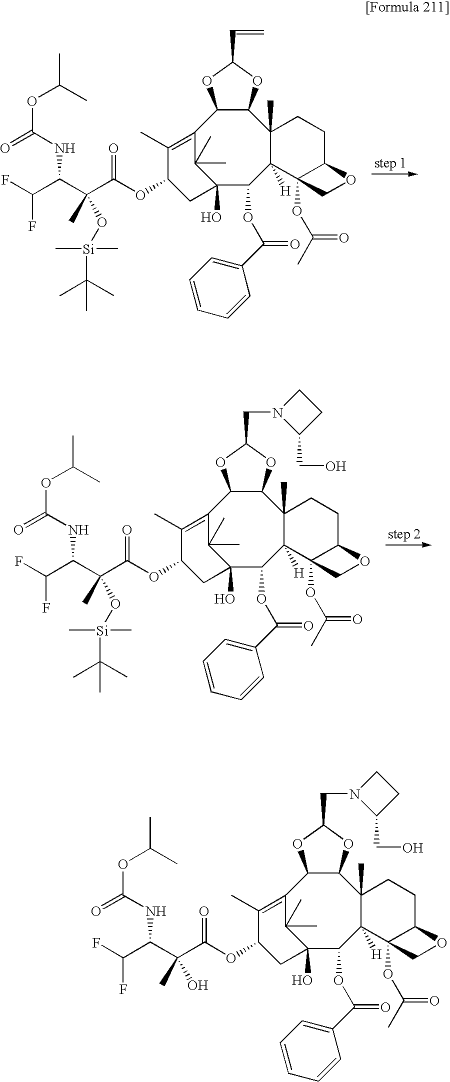

### Step 1: (1S,2S,3R,4S,5R,8R,9S,10R,13S)-4-Acetoxy-2-(benzoyloxy)-5,20-epoxy-1-hydroxy-9,10-{(1S)-2-[(2R)-hydroxymethylazetidino]ethylidenedioxy}tax-11-en-13-yl

### (2R,3R)-2-[(tert-butyldimethylsilyl)oxy]-4,4-difluoro-3-(isopropoxycarbonylamino)-2-methylbutyrate

The compound obtained in Example 105, Step 1 and the compound obtained in Reference Example 26 were used as starting materials to perform the same procedure as that of Example 1 and thereby to obtain the title compound as pale yellow amorphous solid.

¹H-NMR (400 MHz, CDCl₃) δ: 0.15 (3H, s), 0.25 (3H, s), 0.92 (9H, s), 1.15 (3H, d, J=5.9 Hz), 1.20 (3H, d, J=6.1 Hz), 1.24-2.44 (10H, m), 1.33 (3H, s), 1.49 (3H, s), 1.62 (3H, s), 1.73 (3H, s), 1.80 (3H, s), 2.49 (3H, s), 2.79 (1H, dd, J=13.0, 5.4 Hz), 2.86 (1H, d, J=4.9 Hz), 2.95 (1H, dd, J=13.2, 3.9 Hz), 3.10 (1H, q, J=8.3 Hz), 3.44-3.54 (3H, m), 3.66 (1H, dd, J=11.8, 2.0 Hz), 4.12 (1H, d, J=7.1 Hz), 4.23 (1H, d, J=8.3 Hz), 4.29-4.40 (2H, m), 4.72-5.00 (4H, m), 5.17 (1H, d, J=7.1 Hz), 5.89 (1H, td, J=55.7, 4.7 Hz), 5.96 (1H, d, J=4.9 Hz), 6.12 (1H, t, J=8.8 Hz), 7.50 (2H, t, J=7.8 Hz), 7.61 (1H, t, J=7.4 Hz), 8.17 (2H, d, J=8.1 Hz).

FAB-MS; m/z: 993 (M+H)⁺.

### Step 2: (1S,2S,3R,4S,5R,8R,9S,10R,13S)-4-Acetoxy-2-(benzoyloxy)-5,20-epoxy-1-hydroxy-9,10-{(1S)-2-[(2R)-hydroxymethylazetidino]ethylidenedioxy}tax-11-en-13-yl

### (2R,3R)-4,4-difluoro-2-hydroxy-3-(isopropoxycarbonylamino)-2-methylbutyrate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.20 (3H, d, J=6.2 Hz), 1.23 (3H, d, J=6.4 Hz), 1.27 (3H, s), 1.47 (3H, s), 1.61 (3H, s), 1.61-2.06 (8H, m), 1.63 (3H, s), 1.75 (3H, s), 2.18-2.29 (2H, m), 2.42 (3H, s), 2.79 (1H, dd, J=12.7, 5.6 Hz), 2.91-2.95 (2H, m), 3.08 (1H, q, J=8.3 Hz), 3.42-3.53 (3H, m), 3.65 (1H, dd, J=11.4, 2.8 Hz), 4.12 (1H, d, J=7.1 Hz), 4.26 (1H, d, J=8.3 Hz), 4.32 (1H, d, J=8.6 Hz), 4.38-4.48 (1H, m), 4.64 (1H, brs), 4.86-4.95 (3H, m), 5.20 (1H, d, J=7.1 Hz), 5.48 (1H, d, J=10.5 Hz), 6.00 (1H, d, J=4.9 Hz), 6.09 (1H, td, J=55.3, 3.2 Hz), 6.20 (1H, t, J=8.6 Hz), 7.49 (2H, t, J=7.7 Hz), 7.59-7.64 (1H, m), 8.14 (2H, d, J=7.7 Hz).

FAB-MS; m/z: 879 (M+H)⁺.

### Example 140

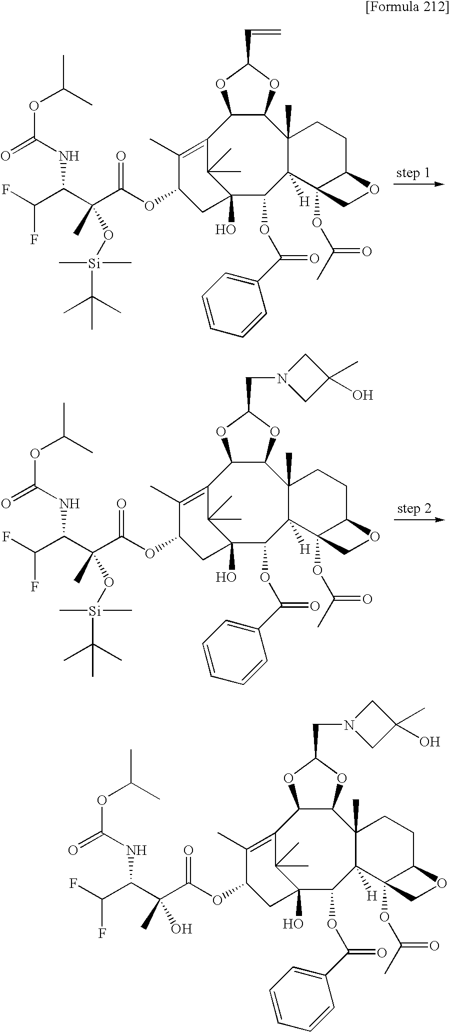

### Step 1: (1S,2S,3R,4S,5R,8R,9S,10R,13S)-4-(Acetoxy)-2-(benzoyloxy)-5,20-epoxy-1-hydroxy-9,10-[(1S)-2-(3-hydroxy-3-methylazetidino)ethylidenedioxy]tax-11-en-13-yl

### (2R,3R)-2-[(tert-butyldimethylsilyl)oxy]-4,4-difluoro-3-(isopropoxycarbonylamino)-2-methylbutyrate

The compound obtained in Example 105, Step 2 and the compound obtained in Reference Example 3 were used as starting materials to perform the same procedure as that of Example 1 and thereby to obtain the title compound as pale yellow amorphous solid.

¹H-NMR (400 MHz, CDCl₃) δ: 0.15 (3H, s), 0.25 (3H, s), 0.92 (9H, s), 1.15 (3H, d, J=6.1 Hz), 1.20 (3H, d, J=6.1 Hz), 1.34 (3H, s), 1.50 (6H, s), 1.59-2.20 (6H, m), 1.62 (3H, s), 1.73 (3H, s), 1.80 (3H, s), 2.49 (3H, s), 2.83-2.88 (2H, m), 2.95 (1H, dd, J=12.3, 3.9 Hz), 3.37-3.49 (4H, m), 3.84 (2H, brs), 4.12 (1H, d, J=7.1 Hz), 4.24 (1H, d, J=8.3 Hz), 4.29-4.40 (2H, m), 4.74-4.93 (3H, m), 4.97 (1H, d, J=10.3 Hz), 5.17 (1H, d, J=7.1 Hz), 5.89 (1H, td, J=55.4, 4.7 Hz), 5.96 (1H, d, J=4.7 Hz), 6.12 (1H, t, J=8.7 Hz), 7.50 (2H, t, J=7.8 Hz), 7.61 (1H, t, J=6.7 Hz), 8.17 (2H, d, J=8.1 Hz).

FAB-MS; m/z: 993 (M+H)⁺.

### Step 2: (1S,2S,3R,4S,5R,8R,9S,10R,13S)-4-(Acetoxy)-2-(benzoyloxy)-5,20-epoxy-1-hydroxy-9,10-[(1S)-2-(3-hydroxy-3-methylazetidino)ethylidenedioxy]tax-11-en-13-yl

### (2R,3R)-4,4-difluoro-2-hydroxy-3-(isopropoxycarbonylamino)-2-methylbutyrate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.19 (3H, d, J=6.2 Hz), 1.23 (3H, d, J=6.4 Hz), 1.28 (3H, s), 1.48 (3H, s), 1.51 (3H, s), 1.60-2.07 (7H, m), 1.61 (3H, s), 1.63 (3H, s), 1.74 (3H, s), 2.22 (1H, dd, J=14.8, 9.7 Hz), 2.42 (3H, s), 2.79 (1H, dd, J=12.5, 5.6 Hz), 2.89-2.94 (2H, m), 3.19-3.29 (2H, m), 3.38-3.46 (2H, m), 4.11 (1H, d, J=7.1 Hz), 4.27 (1H, d, J=8.3 Hz), 4.33 (1H, d, J=8.3 Hz), 4.37-4.48 (1H, m), 4.67 (1H, brs), 4.87-4.94 (3H, m), 5.20 (1H, d, J=6.9 Hz), 5.50 (1H, d, J=10.5 Hz), 5.99 (1H, d, J=4.9 Hz), 6.09 (1H, td, J=55.4, 3.1 Hz), 6.20 (1H, t, J=8.3 Hz), 7.49 (2H, t, J=7.5 Hz), 7.62 (1H, t, J=7.5 Hz), 8.15 (2H, d, J=7.4 Hz).

FAB-MS; m/z: 879 (M+H)⁺.

### Example 141

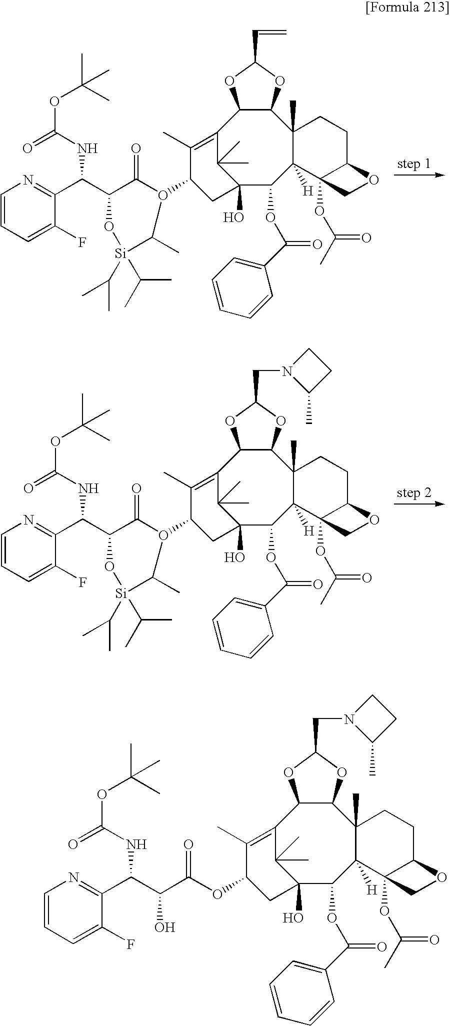

### Step 1: (1S,2S,3R,4S,5R,8R,9S,10R,13S)-4-(Acetoxy)-2-(benzoyloxy)-5,20-epoxy-1-hydroxy-9,10-{(1S)-2-[(2S)-2-methylazetidino]ethylidenedioxy}tax-11-en-13-yl

### (2R,3S)-3-(tert-butoxycarbonylamino)-3-(3-fluoro-2-pyridyl)-2-[(triisopropylsilyl)oxy]propionate

(1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-11-en-13-yl (2R,3S)-3-(tert-butoxycarbonylamino)-3-(3-fluoro-2-pyridyl)-2-[(triisopropylsilyl)oxy]propionate and the compound obtained in Reference Example 61, Step 3 were used as starting materials to perform the same procedure as that of Example 1 and thereby to obtain the title compound as white amorphous solid.

¹H-NMR (400 MHz, CDCl₃) δ: 0.86-0.93 (21H, m), 0.86-2.37 (9H, m), 1.22 (3H, d, J=6.1 Hz), 1.34 (3H, s), 1.38 (9H, s), 1.52 (3H, s), 1.63 (3H, s), 1.79 (3H, s), 2.49 (3H, s), 2.63 (1H, dd, J=12.5, 6.1 Hz), 2.89-2.99 (3H, m), 3.21-3.31 (1H, m), 3.48-3.53 (1H, m), 4.16 (1H, d, J=7.6 Hz), 4.22 (1H, d, J=8.3 Hz), 4.35 (1H, d, J=8.1 Hz), 4.85-4.88 (1H, m), 4.94-4.97 (2H, m), 5.18 (1H, d, J=7.1 Hz), 5.60 (1H, d, J=10.0 Hz), 5.98 (1H, d, J=5.4 Hz), 6.05-6.11 (2H, m), 7.24-7.29 (1H, m), 7.37-7.43 (1H, m), 7.47 (2H, t, J=7.7 Hz), 7.56-7.61 (1H, m), 8.15-8.18 (2H, m), 8.40 (1H, d, J=4.7 Hz).

### Step 2: (1S,2S,3R,4S,5R,8R,9S,10R,13S)-4-(Acetoxy)-2-(benzoyloxy)-5,20-epoxy-1-hydroxy-9,10-{(1S)-2-[(2S)-2-methylazetidino]ethylidenedioxy}tax-11-en-13-yl

### (2R,3S)-3-(tert-butoxycarbonylamino)-3-(3-fluoro-2-pyridyl)-2-hydroxypropionate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 1.19-2.15 (9H, m), 1.22 (3H, d, J=6.1 Hz), 1.28 (3H, s), 1.40 (9H, s), 1.50 (3H, s), 1.62 (3H, s), 1.78 (3H, s), 2.34 (3H, s), 2.40-2.49 (1H, m), 2.64 (1H, dd, J=12.5, 6.4 Hz), 2.89-2.97 (3H, m), 3.23-3.32 (1H, m), 3.48-3.55 (1H, m), 4.13 (1H, d, J=7.1 Hz), 4.23 (1H, d, J=8.3 Hz), 4.33 (1H, d, J=8.3 Hz), 4.68 (1H, d, J=2.7 Hz), 4.87-4.93 (2H, m), 5.22 (1H, d, J=6.9 Hz), 5.65 (1H, d, J=8.3 Hz), 6.00 (1H, d, J=5.1 Hz), 6.08 (1H, t, J=8.3 Hz), 6.21 (1H, d, J=8.3 Hz), 7.28-7.33 (1H, m), 7.42-7.50 (3H, m), 7.57-7.62 (1H, m), 8.11-8.15 (2H, m), 8.40 (1H, d, J=4.7 Hz).

FAB-MS; m/z: 908 (M+H)⁺.

### Example 142

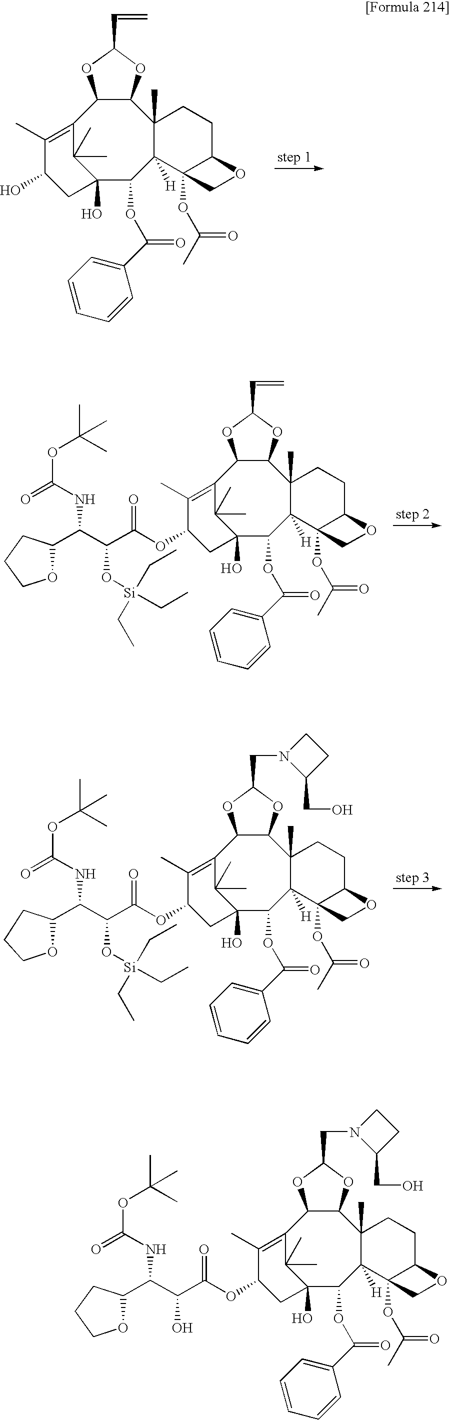

### Step 1: (1S,2S,3R,4S,5R,8R,9S,10R,13S)-4-(Acetoxy)-2-(benzoyloxy)-5,20-epoxy-1-hydroxy-9,10-[(1S)-2-propenylidenedioxy]tax-1′-en-13-yl

### (2R,3S)-3-(tert-butoxycarbonyl)-3-[(2R)-tetrahydrofuran-2-yl]-2-[(triethylsilyl)oxy]propionate

(1S,2S,3R,4S,5R,8R,9S,10R,13S)-4-(Acetoxy)-2-(benzoyloxy)-1,13-dihydroxy-5,20-epoxy-9,10-[(1S)-2-propenylidenedioxy]tax-11-ene and the compound obtained in Reference Example 64, Step 4 were used as starting materials to perform the same procedure as that of Example 31, Step 1 and thereby to obtain the title compound as white amorphous solid.

¹H-NMR (400 MHz, CDCl₃) δ: 0.62-0.70 (6H, m), 0.99 (9H, t, J=7.6 Hz), 1.32 (3H, s), 1.41 (9H, s), 1.47-2.27 (11H, m), 1.51 (3H, s), 1.64 (3H, s), 1.79 (3H, s), 2.39 (3H, s), 2.96 (1H, d, J=5.4 Hz), 3.70-3.77 (1H, m), 3.83-3.90 (1H, m), 3.92-3.98 (1H, m), 4.17-4.23 (2H, m), 4.34 (2H, d, J=8.3 Hz), 4.42 (1H, d, J=3.7 Hz), 4.91-4.96 (2H, m), 5.20 (1H, d, J=6.3 Hz), 5.27 (1H, d, J=6.8 Hz), 5.45 (1H, d, J=10.5 Hz), 5.57 (1H, d, J=17.1 Hz), 5.94-6.04 (2H, m), 6.07 (1H, t, J=8.5 Hz), 7.48 (2H, t, J=7.7 Hz), 7.60 (1H, t, J=7.4 Hz), 8.15 (2H, d, J=8.5 Hz).

ESI-MS; m/z: 940 (M+H)⁺.

### Step 2: (1S,2S,3R,4S,5R,8R,9S,10R,13S)-4-(Acetoxy)-2-(benzoyloxy)-5,20-epoxy-1-hydroxy-9,10-{(1 S)-2-[(2S)-2-hydroxymethylazetidino]ethylidenedioxy}tax-11-en-13-yl

### (2R,3S)-3-(tert-butoxycarbonyl)-3-[(2R)-tetrahydrofuran-2-yl]-2-[(triethylsilyl)oxy]propionate

The compound obtained in Step 1 mentioned above and the compound obtained in Reference Example 52 were used as starting materials to perform the same procedure as that of Example 1 and thereby to obtain the title compound as white amorphous solid.

¹H-NMR (400 MHz, CDCl₃) δ: 0.60-0.70 (6H, m), 0.98 (9H, t, J=7.9 Hz), 1.30 (3H, s), 1.41 (9H, s), 1.50 (3H, s), 1.55-2.31 (14H, m), 1.59 (3H, s), 1.77 (3H, s), 2.39 (3H, s), 2.80 (1H, dd, J=12.9, 4.6 Hz), 2.88 (1H, dd, J=12.8, 5.0 Hz), 2.94 (1H, d, J=4.9 Hz), 3.09 (1H, q, J=8.2 Hz), 3.39-3.52 (3H, m), 3.63 (1H, dd, J=11.2, 2.9 Hz), 3.69-3.77 (1H, m), 3.83-3.98 (2H, m), 4.05-4.12 (1H, m), 4.16 (1H, d, J=7.1 Hz), 4.19 (1H, d, J=8.3 Hz), 4.34 (1H, d, J=8.5 Hz), 4.42 (1H, d, J=3.7 Hz), 4.84 (1H, t, J=4.9 Hz), 4.91-4.96 (2H, m), 5.20 (1H, d, J=7.1 Hz), 5.95 (1H, d, J=5.1 Hz), 6.05 (1H, t, J=9.0 Hz), 7.48 (2H, t, J=7.8 Hz), 7.60 (1H, t, J=7.4 Hz), 8.14 (2H, d, J=8.0 Hz).

ESI-MS; m/z: 1013 (M+H)⁺.

### Step 3: (1S,2S,3R,4S,5R,8R,9S,10R,13S)-4-(Acetoxy)-2-(benzoyloxy)-5,20-epoxy-1-hydroxy-9,10-{(1S)-2-[(2S)-2-hydroxymethylazetidino]ethylidenedioxy}tax-11-en-13-yl

### (2R,3R)-3-(tert-butoxycarbonyl)-2-hydroxy-3-[(2R)-tetrahydrofuran-2-yl]propionate

The compound obtained in Step 2 mentioned above was used as a starting material to perform the same procedure as that of Example 31, Step 2 and thereby to obtain the title compound as white amorphous solid.

¹H-NMR (400 MHz, CDCl₃) δ: 1.25 (3H, s), 1.42 (9H, s), 1.49 (3H, s), 1.59 (3H, s), 1.71-2.44 (14H, m), 1.83 (3H, s), 2.31 (3H, s), 2.83 (1H, dd, J=12.7, 5.1 Hz), 2.89 (1H, dd, J=12.9, 5.4 Hz), 2.93 (1H, d, J=5.4 Hz), 3.10 (1H, q, J=8.3 Hz), 3.38-3.53 (3H, m), 3.65 (1H, dd, J=11.1, 3.1 Hz), 3.70-3.78 (2H, m), 3.83-3.92 (1H, m), 4.10-4.25 (4H, m), 4.32 (1H, d, J=8.1 Hz), 4.47 (1H, s), 4.88 (1H, t, J=5.0 Hz), 4.93 (1H, s), 5.19-5.28 (2H, m), 5.93-6.02 (2H, m), 7.47 (2H, t, J=7.7 Hz), 7.60 (1H, t, J=7.4 Hz), 8.12 (2H, d, J=7.1 Hz).

ESI-MS; m/z: 899 (M+H)⁺.

### Example 143

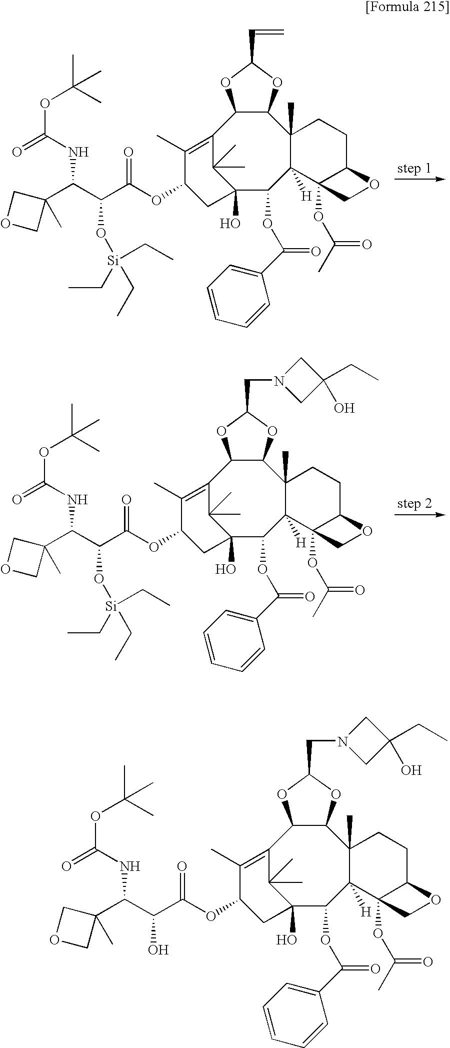

### Step 1: (1S,2S,3R,4S,5R,8R,9S,10R,13S)-4-(Acetoxy)-2-(benzoyloxy)-5,20-epoxy-9,10-[(1S)-2-(3-ethyl-3-hydroxyazetidino)ethylidenedioxy]-hydroxytax-11-en-13-yl

### (2R,3S)-3-(tert-butoxycarbonylamino)-3-(3-methyl-3-oxetanyl)-2-[(triethylsilyl)oxy]propionate

The compound obtained in Example 123, Step 1 and the compound obtained in Reference Example 4, Step 2 were used as starting materials to perform the same procedure as that of Example 1 and thereby to obtain the title compound as white amorphous solid.

¹H-NMR (400 MHz, CDCl₃) δ: 0.55-0.71 (6H, m), 0.97 (3H, t, J=7.3 Hz), 0.98 (9H, t, J=8.1 Hz), 1.27-2.21 (10H, m), 1.29 (3H, s), 1.39 (9H, s), 1.50 (3H, s), 1.53 (3H, s), 1.60 (3H, s), 1.76 (3H, s), 2.35 (3H, s), 2.78-2.94 (3H, m), 3.17 (2H, t, J=7.7 Hz), 3.43-3.51 (2H, m), 3.96 (1H, dd, J=10.4, 1.8 Hz), 4.14 (1H, d, J=8.1 Hz), 4.18-4.28 (3H, m), 4.33 (1H, d, J=8.1 Hz), 4.59 (1H, d, J=2.0 Hz), 4.64 (1H, d, J=5.9 Hz), 4.74 (1H, d, J=6.8 Hz), 4.86 (1H, t, J=4.9 Hz), 4.91 (1H, brs), 5.17 (1H, d, J=6.8 Hz), 5.33 (1H, d, J=10.5 Hz), 5.95 (1H, d, J=5.1 Hz), 6.02 (1H, t, J=8.9 Hz), 7.49 (2H, t, J=7.6 Hz), 7.61 (1H, tt, J=7.3, 1.5 Hz), 8.11-8.16 (2H, m).

### Step 2: (1S,2S,3R,4S,5R,8R,9S,10R,13S)-4-(Acetoxy)-2-(benzoyloxy)-5,20-epoxy-9,10-[(1S)-2-(3-ethyl-3-hydroxyazetidino)ethylidenedioxy]-1-hydroxytax-11-en-13-yl

### (2R,3S)-3-(tert-butoxycarbonylamino)-2-hydroxy-3-(3-methyl-3-oxetanyl)propionate

The compound obtained in Step 1 mentioned above was used as a starting material to perform the same procedure as that of Example 31, Step 2 and thereby to obtain the title compound as white amorphous solid.

¹H-NMR (400 MHz, CDCl₃) δ: 0.97 (3H, t, J=7.6 Hz), 1.24 (3H, s), 1.37-2.41 (10H, m), 1.41 (9H, s), 1.49 (3H, s), 1.54 (3H, s), 1.61 (3H, s), 1.80 (3H, s), 2.31 (3H, s), 2.80 (1H, dd, J=12.3, 6.0 Hz), 2.89-2.95 (2H, m), 3.15 (2H, t, J=8.8 Hz), 3.46 (2H, t, J=7.4 Hz), 4.12 (1H, d, J=7.1 Hz), 4.20-4.34 (6H, m), 4.48 (1H, s), 4.67 (1H, d, J=6.1 Hz), 4.80 (1H, d, J=7.1 Hz), 4.89-4.95 (2H, m), 5.24 (1H, d, J=6.8 Hz), 5.36 (1H, d, J=10.5 Hz), 6.00 (1H, d, J=4.9 Hz), 6.07 (1H, t, J=8.5 Hz), 7.48 (2H, t, J=7.7 Hz), 7.61 (1H, tt, J=7.3, 1.2 Hz), 8.10-8.15 (2H, m).

FAB-MS; m/z: 913 (M+H)⁺.

### Test Example 1

Three types of human tumor cell lines (all of the cells were obtained from American Type Culture Collection) were seeded on 96-well microplates as follows.

NCI-H460 (human lung cancer cell line): 5.0×10² cells/190 μL/well
HCT-15 (P glycoprotein (P-gp) expressing human colon cancer cell line, J. Natl. Cancer Inst., 2001, 93:1234-1245): 1.0×10³ cells/190 μL/well
HCT116 (human colon cancer cell line): 1.0×10³ cells/190 μL/well
On the next day, each test compound was dissolved in 100% dimethyl sulfoxide (DMSO) at a concentration of 10 mg/mL. This solution was diluted with 0.01% Cremophor
EL-PBS(−) (Cremophor EL: Sigma Chemical Co., St. Louis, Mo., USA, PBS: Invitrogen Corp., Carlsbad, Calif., USA), and 10 μL of the diluted solution was added to each well (final concentration: 100 to 0.000381 ng/mL). The cells were cultured for three days, and then 20 μ/L of a 5 mg/mL solution of 3-(4,5-dimethylthiazol-2-yl)-2,5-diphenyl-2H-tetrazolium bromide (MTT, Sigma Chemical Co.) was added to each well. The cells were further cultured for 4 hours, and then the medium was removed by centrifugation. By adding 150 μL of DMSO to each well, the produced formazan product was dissolved. The absorbance of the solution was measured at 540 nm. The drug concentration suppressing cell proliferation of the drug addition group to 50% of the cell proliferation of the drug free group (GI50 value, ng/mL) was calculated, and used as an index of anti-cell activity.

The results are shown in Table 1. The compounds of the present invention showed significant antitumor effect against the cancer cell lines including the drug resistant cells.

TABLE 1

| | NCI-H460 | HCT-15 | HCT116 |
|---|---|---|---|
| | (GI50 value, | (GI50 value, | (GI50 value, |
| | ng/mL) | ng/mL) | ng/mL) |
| Paclitaxel | 1.87 | >100 | 1.71 |
| Compound of | 0.19 | 1.14 | 0.244 |
| Example 1 | | | |
| Compound of | 0.606 | 2.31 | 0.669 |
| Example 2 | | | |
| Compound of | 0.162 | 8.08 | 0.229 |
| Example 5 | | | |
| Compound of | 0.172 | 7.94 | 0.183 |
| Example 6 | | | |
| Compound of | 0.128 | 0.236 | 0.155 |
| Example 8 | | | |
| Compound of | 0.109 | 4.21 | 0.173 |
| Example 11 | | | |

### Test Example 2

Mice (C57BL/6, Charles River Japan) were subcutaneously transplanted with B16 melanoma BL6 (Cancer Res., Poste, G., Fidler, I. J. et al., 1980, 40:1636; Bioorg. Med. Chem. Lett., Takeda, Y. et al., 2003, 13:185-190), or M5076 (obtained from Japanese Foundation for Cancer Research), which is a histiocytoma expressing P-gp. Four days after the transplantation in the case of B16 melanoma BL6, or 8 to 10 days after the transplantation in the case of M5076, each test compound suspended in a 0.5% aqueous solution of carboxymethylcellulose sodium, or dissolved in EtOH:Tween 80:5% glucose=5:5:90 (v/v %) was orally (po) or intravenously (iv) administered to the mice. Ten days after the administration in the case of B16 melanoma BL6, or 8 to 10 days after the administration in the case of M5076, the mice were dissected, and tumor weights were measured. The antitumor effect was calculated in accordance with the following equation.

[1−(Average tumor weight of compound administration group/average tumor weight of solvent administration group)]×100

An antitumor effect of 58% or more was considered effective (Int. J. Cancer, 1989, 43:637-44), and the maximum tolerance dose (MTD, maximum dose not causing death, mg/kg) and minimum effective dose (MED, mg/kg) showing effectiveness were obtained, respectively. The therapeutic index was calculated in accordance with the equation: MTD/MED.

The results are shown in Table 2. ND means that the test was not performed. The effective dose range means the range of from MED to MTD.

TABLE 2

| | B16 Melanoma BL6 | | | | M5076 | | | |
|---|---|---|---|---|---|---|---|---|
| | po | | iv | | po | | iv | |
| | Effective dose | Therapeutic | Effective dose | Therapeutic | Effective dose | Therapeutic | Effective dose | Therapeutic |
| | range (mg/kg) | index | range (mg/kg) | index | range (mg/kg) | index | range (mg/kg) | index |
| Compound of | 5.88-12.0 | 2.0 | 4.12-12.0 | 2.9 | 8.40 | 1.0 | 8.40 | 1.0 |
| WO01/27115, | | | | | | | | |
| Example 7 | | | | | | | | |
| Compound of | 8.61-25.0 | 2.9 | ND | ND | ND | ND | ND | ND |
| Example 1 | | | | | | | | |
| Compound of | 6.71-19.6 | 2.9 | 7.21-21.0 | 2.9 | 13.7-28 | 2.0 | 14.7 | 1.0 |
| Example 2 | | | | | | | | |
| Compound of | 14.7-87.6 | 6.0 | ND | ND | 30.0 | 1.0 | ND | ND |
| Example 5 | | | | | | | | |
| Compound of | 9.10-38.0 | 4.2 | ND | ND | 18.6-38.0 | 2.0 | ND | ND |
| Example 6 | | | | | | | | |
| Compound of | 10.3-30.0 | 2.9 | ND | ND | 21.0-30.0 | 1.4 | ND | ND |
| Example 8 | | | | | | | | |
| Compound of | 10.3-61.3 | 6.0 | ND | ND | ND | ND | ND | ND |
| Example 9 | | | | | | | | |
| Compound of | 7.07-42.0 | 5.9 | ND | ND | ND | ND | ND | ND |
| Example 11 | | | | | | | | |
| Compound of | 11.6-48.0 | 4.1 | ND | ND | ND | ND | ND | ND |
| Example 12 | | | | | | | | |
| Compound of | 7.63-45.0 | 5.9 | ND | ND | ND | ND | ND | ND |
| Example 21 | | | | | | | | |
| Compound of | 8.40-35.0 | 4.2 | 5.88-35.0 | 6.0 | 17.2-35 | 2.0 | 12.0-24.5 | 2.0 |
| Example 22 | | | | | | | | |
| Compound of | 20.6-60.0 | 2.9 | ND | ND | ND | ND | ND | ND |
| Example 23 | | | | | | | | |
| Compound of | 13.2-55.0 | 4.2 | ND | ND | ND | ND | ND | ND |
| Example 28 | | | | | | | | |
| Compound of | 8.82-18.0 | 2.0 | 3.43-10.0 | 2.9 | ND | ND | 10.0 | 1.0 |
| Example 31 | | | | | | | | |
| Compound of | ND | ND | 10.1-42.0 | 4.2 | ND | ND | ND | ND |
| Example 34 | | | | | | | | |
| Compound of | 14.7-30.0 | 2.0 | 7.21-30.0 | 4.2 | ND | ND | 14.7-21.0 | 1.4 |
| Example 37 | | | | | | | | |
| Compound of | ND | ND | 5.52-23.0 | 4.2 | ND | ND | ND | ND |
| Example 40 | | | | | | | | |
| Compound of | 8.58-25.0 | 2.9 | 5.29-22.0 | 4.2 | ND | ND | 17.5-25.0 | 1.4 |
| Example 41 | | | | | | | | |

### Test Example 3

Solubility of each compound was determined in the first solution for disintegration test according to Japanese Pharmacopoeia (henceforth referred to as the first solution, solution obtained by adding 7 mL of hydrochloric acid and water to 2.00 g of sodium chloride to dissolve sodium chloride and fill up the solution to 1000 mL, pH 1.2) and the second solution for disintegration test according to Japanese Pharmacopoeia (henceforth referred to as the second solution, solution obtained by adding 118 mL of 0.2 mol/L solution of sodium hydroxide to 6.80 g of potassium dihydrogenphosphate, adding water to the mixture to dissolve potassium dihydrogenphosphate and filling up the solution to 1000 mL, pH 6.8).

Each test compound was dissolved in 100% dimethyl sulfoxide (DMSO) at a concentration of 10 mM, and 50 μL of the solution was lyophilized. The lyophilized test compound was added with 250 μL of the first solution or second solution, and the mixture was stored at room temperature for 12 hours or longer under light shielding, and filtered (0.7 μm). The filtrate was taken in a volume of 90 μL, and added with 50% DMSO (90 μL) to obtain a two-fold diluted solubility sample. The two-fold dilution solubility sample was taken in a volume of 20 μL, and added with 50% DMSO (180 μL) to obtain a 20-fold diluted solubility sample. Solubility of each test sample was determined by quantifying the test compound in the resulting diluted solubility test samples using high-performance liquid chromatography.

The results are shown in Table 3.

TABLE 3

Solubility (μg/mL)

| | pH 1.2 | pH 6.8 |
|---|---|---|
| Paclitaxel | <4 | <4 |
| Compound of WO01/27115, | 760-950 | 36 |
| Example 7 | | |
| Compound of Example 1 | 1200 | 170 |
| Compound of Example 2 | 1100 | 170 |
| Compound of Example 5 | 1000 | 140 |
| Compound of Example 6 | 690 | 87 |
| Compound of Example 8 | 600 | 10 |
| Compound of Example 9 | 1400 | 130 |
| Compound of Example 11 | 1300 | 170 |
| Compound of Example 12 | 1100 | 92 |
| Compound of Example 21 | >1000 | 150 |
| Compound of Example 22 | 1100 | 85 |
| Compound of Example 23 | 1400 | 360 |
| Compound of Example 28 | 1300 | 18 |
| Compound of Example 31 | 1000 | 160 |
| Compound of Example 34 | 830 | 310 |
| Compound of Example 37 | 670 | 210 |
| Compound of Example 40 | 1000 | 150 |
| Compound of Example 41 | 910 | 370 |

### INDUSTRIAL APPLICABILITY

The compounds of the present invention have high antitumor effect against cancer cells including drug resistant cells, and accordingly, they can be used as potent anticancer agents. Furthermore, the compounds of the present invention have a wide effective dose range, thereby an optimum dosage wide apart from MTD can be expected, and therefore, they can be used as safe anticancer agents less likely to cause side effects.

1. A compound represented by the general formula (1) or a salt thereof, or a solvate thereof: 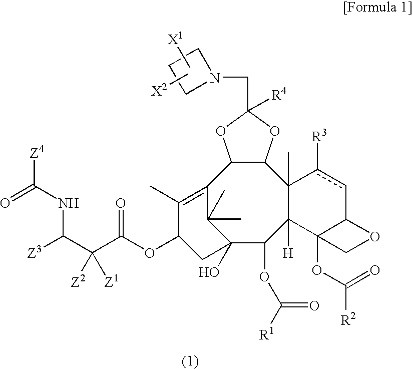
[wherein X¹ and X² independently represent hydrogen atom, a halogen atom, hydroxyl group, cyano group, carboxy group, an alkoxy group which may be substituted, an alkoxycarbonyl group, an alkyl group which may be substituted, an alkenyl group which may be substituted, an alkynyl group which may be substituted, a phenyl group which may be substituted, an alkanoyl group which may be substituted, a carbamoyl group which may be substituted, a carbamoyloxy group which may be substituted, an alkylsulfonyl group which may be substituted, an aminosulfonyl group which may be substituted, an amino group which may be substituted, or a 4- to 6-membered saturated heterocyclic group, or
X¹ and X² may together form oxo group or a ═N—OY group (Y represents hydrogen atom, or an alkyl group which may be substituted), or X¹ and X² may form a 4- to 6-membered saturated hydrocarbon ring binding as a spiro ring system or a condensed ring system, or a 5- or 6-membered saturated heterocyclic ring binding as a spiro ring system or a condensed ring system together with the carbon atom or atoms to which X¹ and X² bind,
R¹ represents a phenyl group which may be substituted,
R² represents an alkyl group which may be substituted, an alkenyl group which may be substituted, or an alkoxy group which may be substituted,
R³ represents hydrogen atom, a halogen atom, hydroxyl group, or an alkoxy group which may be substituted,
R⁴ represents hydrogen atom, or an alkyl group which may be substituted,
Z¹ and Z² independently represent hydrogen atom, a halogen atom, hydroxyl group, an alkoxy group which may be substituted, or an alkyl group which may be substituted,
Z³ represents cyano group, an alkyl group which may be substituted, an alkenyl group which may be substituted, an alkynyl group which may be substituted, a phenyl group which may be substituted, or a 4- to 6-membered saturated or unsaturated heterocyclic group which may be substituted,
Z⁴ represents an alkyl group which may be substituted, an alkenyl group which may be substituted, an alkynyl group which may be substituted, a phenyl group which may be substituted, an aralkyl group which may be substituted, a 5- or 6-membered aromatic heterocyclic group which may be substituted, or an alkoxy group which may be substituted, and
the 6- and 7-positions of the partial structure represented by the following formula (2) 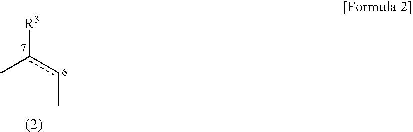
may be bound with a double bond (when the bond is a double bond, R³ is hydrogen atom).

2. The compound or a salt thereof, or a solvate thereof according to claim 1, wherein X¹ and X² independently represent hydrogen atom, a halogen atom, hydroxyl group, cyano group, an alkoxy group which may be substituted, an alkyl group which may be substituted, a carbamoyl group which may be substituted, a carbamoyloxy group which may be substituted, an alkylsulfonyl group which may be substituted, or an amino group which may be substituted, or X¹ and X² together represent a ═N—OY group (Y represents hydrogen atom, or an alkyl group which may be substituted).

3. The compound or a salt thereof, or a solvate thereof according to claim 1, wherein R² is an alkyl group which may be substituted.

4. The compound or a salt thereof, or a solvate thereof according to claim 1, wherein R³ is hydrogen atom.

5. The compound or a salt thereof, or a solvate thereof according to claim 1, wherein R⁴ is hydrogen atom.

6. The compound or a salt thereof, or a solvate thereof according to claim 1, wherein Z¹ is hydroxyl group.

7. The compound or a salt thereof, or a solvate thereof according to claim 1, wherein Z² is hydrogen atom, or an alkyl group which may be substituted

8. The compound or a salt thereof, or a solvate thereof according to claim 1, wherein Z³ is an alkyl group which may be substituted, or a 4- to 6-membered saturated or unsaturated heterocyclic group which may be substituted.

9. The compound or a salt thereof, or a solvate thereof according to claim 1, wherein Z⁴ is a phenyl group which may be substituted, or an alkoxy group which may be substituted.

10. The compound or a salt thereof, or a solvate thereof according to claim 1, wherein the 6- and 7-positions of the partial structure represented by the formula (2) are bonded with a single bond.

11. The compound or a salt thereof, or a solvate thereof according to claim 1, which has the absolute configuration represented by the general formula (3): 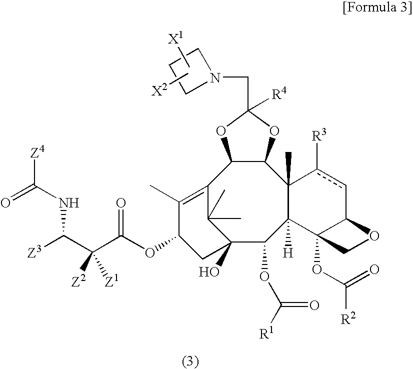
(wherein X¹, X², R¹ to R⁴, and Z¹ to Z⁴ have the same meanings as those defined above).

12. A medicament comprising the compound or a salt thereof, or a solvate thereof according to claim 1 as an active ingredient.

13. An anticancer agent comprising the compound or a salt thereof, or a solvate thereof according to claim 1 as an active ingredient.

14. A pharmaceutical composition comprising the compound or a salt thereof, or a solvate thereof according to claim 1, and a pharmaceutically acceptable carrier.

15. Use of the compound or a salt thereof, or a solvate thereof according to claim 1 for manufacture of a medicament.

16. Use of the compound or a salt thereof, or a solvate thereof according to claim 1 for manufacture of an anticancer agent.

17. A method for therapeutic treatment of cancer, which comprises administering the compound or a salt thereof, or a solvate thereof according to claim 1.
